SEQUENCE LISTING (151 sequences)

SEQ ID NO: 1 - DNA, 1701 bp, Artificial Sequence
  Synthetic Construct
atgtgtgcgacctcttctcaatttactcagattaccgagcataattcccgtcgttccgcaaactatcagccaaacctgtggaatttcgaattcctgcaatccctggagaacgacctgaaagtggaaaagctggaggagaaagcgaccaaactggaggaagaagttcgctgcatgatcaaccgtgtagacacccagccgctgtccctgctggagctgatcgacgatgtgcagcgcctgggtctgacctacaaatttgaaaaagacatcattaaagccctggaaaacatcgtactgctggacgaaaacaaaaagaacaaatctgacctgcacgcaaccgctctgtctttccgtctgctgcgtcagcacggtttcgaggtttctcaggatgtttttgagcgtttcaaggataaagaaggtggtttcagcggtgaactgaaaggtgacgtccaaggcctgctgagcctgtatgaagcgtcttacctgggtttcgagggtgagaacctgctggaggaggcgcgtaccttttccatcacccacctgaagaacaacctgaaagaaggcattaataccaaggttgcagaacaagtgagccacgccctggaactgccatatcaccagcgtctgcaccgtctggaggcacgttggttcctggataaatacgaaccgaaagaaccgcatcaccagctgctgctggagctggcgaagctggattttaacatggtacagaccctgcaccagaaagagctgcaagatctgtcccgctggtggaccgagatgggcctggctagcaaactggattttgtacgcgaccgcctgatggaagtttatttctgggcactgggtatggcgccagacccgcagtttggtgaatgtcgcaaagctgttactaaaatgtttggtctggtgacgatcatcgatgacgtgtatgacgtttatggcactctggacgaactgcaactgttcaccgatgctgtagagcgctgggacgttaacgctattaacaccctgccggactatatgaaactgtgtttcctggcactgtacaacaccgttaacgacacgtcctattctattctgaaagagaaaggtcataacaacctgtcctatctgacgaaaagctggcgtgaactgtgcaaagcctttctgcaagaggcgaaatggtccaacaacaaaattatcccggctttctccaagtacctggaaaacgccagcgtttcctcctccggtgtagcgctgctggcgccgtcttacttttccgtatgccagcagcaggaagacatctccgaccacgcgctgcgttccctgaccgacttccatggtctggtgcgttctagctgcgttatcttccgcctgtgcaacgatctggccacctctgcggcggagctggaacgtggcgagactaccaattctatcattagctacatgcacgaaaacgatggtaccagcgaggaacaggcccgcgaagaactgcgtaaactgatcgacgccgaatggaaaaagatgaatcgtgaacgcgttagcgactccaccctgctgcctaaagcgttcatggaaatcgcagttaacatggcacgtgtttcccactgcacctaccagtatggcgatggtctgggtcgcccagactacgcgactgaaaaccgcatcaaactgctgctgattgaccctttcccgattaaccagctgatgtatgtctaactgcag

SEQ ID NO: 2 - DNA, 6080 bp, Artificial Sequence
  Synthetic Construct
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatgtgtgcgacctcttctcaatttactcagattaccgagcataattcccgtcgttccgcaaactatcagccaaacctgtggaatttcgaattcctgcaatccctggagaacgacctgaaagtggaaaagctggaggagaaagcgaccaaactggaggaagaagttcgctgcatgatcaaccgtgtagacacccagccgctgtccctgctggagctgatcgacgatgtgcagcgcctgggtctgacctacaaatttgaaaaagacatcattaaagccctggaaaacatcgtactgctggacgaaaacaaaaagaacaaatctgacctgcacgcaaccgctctgtctttccgtctgctgcgtcagcacggtttcgaggtttctcaggatgtttttgagcgtttcaaggataaagaaggtggtttcagcggtgaactgaaaggtgacgtccaaggcctgctgagcctgtatgaagcgtcttacctgggtttcgagggtgagaacctgctggaggaggcgcgtaccttttccatcacccacctgaagaacaacctgaaagaaggcattaataccaaggttgcagaacaagtgagccacgccctggaactgccatatcaccagcgtctgcaccgtctggaggcacgttggttcctggataaatacgaaccgaaagaaccgcatcaccagctgctgctggagctggcgaagctggattttaacatggtacagaccctgcaccagaaagagctgcaagatctgtcccgctggtggaccgagatgggcctggctagcaaactggattttgtacgcgaccgcctgatggaagtttatttctgggcactgggtatggcgccagacccgcagtttggtgaatgtcgcaaagctgttactaaaatgtttggtctggtgacgatcatcgatgacgtgtatgacgtttatggcactctggacgaactgcaactgttcaccgatgctgtagagcgctgggacgttaacgctattaacaccctgccggactatatgaaactgtgtttcctggcactgtacaacaccgttaacgacacgtcctattctattctgaaagagaaaggtcataacaacctgtcctatctgacgaaaagctggcgtgaactgtgcaaagcctttctgcaagaggcgaaatggtccaacaacaaaattatcccggctttctccaagtacctggaaaacgccagcgtttcctcctccggtgtagcgctgctggcgccgtcttacttttccgtatgccagcagcaggaagacatctccgaccacgcgctgcgttccctgaccgacttccatggtctggtgcgttctagctgcgttatcttccgcctgtgcaacgatctggccacctctgcggcggagctggaacgtggcgagactaccaattctatcattagctacatgcacgaaaacgatggtaccagcgaggaacaggcccgcgaagaactgcgtaaactgatcgacgccgaatggaaaaagatgaatcgtgaacgcgttagcgactccaccctgctgcctaaagcgttcatggaaatcgcagttaacatggcacgtgtttcccactgcacctaccagtatggcgatggtctgggtcgcccagactacgcgactgaaaaccgcatcaaactgctgctgattgaccctttcccgattaaccagctgatgtatgtctaactgcagctggtaccatatgggaattcgaagctttctagaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 3 - DNA, 37 bp, Artificial Sequence
  Synthetic Construct
cgtgagatcatatgtgtgcgacctcttctcaatttac

SEQ ID NO: 4 - DNA, 38 bp, Artificial Sequence
  Synthetic Construct
cggtcgacggatccctgcagttagacatacatcagctg

SEQ ID NO: 5 - DNA, 7404 bp, Artificial Sequence
  Synthetic Construct
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatccggatatagttcctcctttcagcaaaaaacccctcaagacccgtttagaggccccaaggggttatgctagttattgctcagcggtggcagcagccaactcagcttcctttcgggctttgttagcagccggatccctgcagttagacatacatcagctggttaatcgggaaagggtcaatcagcagcagtttgatgcggttttcagtcgcgtagtctgggcgacccagaccatcgccatactggtaggtgcagtgggaaacacgtgccatgttaactgcgatttccatgaacgctttaggcagcagggtggagtcgctaacgcgttcacgattcatctttttccattcggcgtcgatcagtttacgcagttcttcgcgggcctgttcctcgctggtaccatcgttttcgtgcatgtagctaatgatagaattggtagtctcgccacgttccagctccgccgcagaggtggccagatcgttgcacaggcggaagataacgcagctagaacgcaccagaccatggaagtcggtcagggaacgcagcgcgtggtcggagatgtcttcctgctgctggcatacggaaaagtaagacggcgccagcagcgctacaccggaggaggaaacgctggcgttttccaggtacttggagaaagccgggataattttgttgttggaccatttcgcctcttgcagaaaggctttgcacagttcacgccagcttttcgtcagataggacaggttgttatgacctttctctttcagaatagaataggacgtgtcgttaacggtgttgtacagtgccaggaaacacagtttcatatagtccggcagggtgttaatagcgttaacgtcccagcgctctacagcatcggtgaacagttgcagttcgtccagagtgccataaacgtcatacacgtcatcgatgatcgtcaccagaccaaacattttagtaacagctttgcgacattcaccaaactgcgggtctggcgccatacccagtgcccagaaataaacttccatcaggcggtcgcgtacaaaatccagtttgctagccaggcccatctcggtccaccagcgggacagatcttgcagctctttctggtgcagggtctgtaccatgttaaaatccagcttcgccagctccagcagcagctggtgatgcggttctttcggttcgtatttatccaggaaccaacgtgcctccagacggtgcagacgctggtgatatggcagttccagggcgtggctcacttgttctgcaaccttggtattaatgccttctttcaggttgttcttcaggtgggtgatggaaaaggtacgcgcctcctccagcaggttctcaccctcgaaacccaggtaagacgcttcatacaggctcagcaggccttggacgtcacctttcagttcaccgctgaaaccaccttctttatccttgaaacgctcaaaaacatcctgagaaacctcgaaaccgtgctgacgcagcagacggaaagacagagcggttgcgtgcaggtcagatttgttctttttgttttcgtccagcagtacgatgttttccagggctttaatgatgtctttttcaaatttgtaggtcagacccaggcgctgcacatcgtcgatcagctccagcagggacagcggctgggtgtctacacggttgatcatgcagcgaacttcttcctccagtttggtcgctttctcctccagcttttccactttcaggtcgttctccagggattgcaggaattcgaaattccacaggtttggctgatagtttgcggaacgacgggaattatgctcggtaatctgagtaaattgagaagaggtcgcacacatatgacgaccttcgatatggccgctgctgtgatgatgatgatgatgatgatgatgatggcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 6 - DNA, 41 bp, Artificial Sequence
  Synthetic Construct
catatgaaagcttgtatcgattaaataaggaggaataaacc

SEQ ID NO: 7 - DNA, 6266 bp, Artificial Sequence
  Synthetic Construct
cccgtcttactgtcgggaattcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgtatcgattaaataaggaggaataaaccatgtgtgcgacctcttctcaatttactcagattaccgagcataattcccgtcgttccgcaaactatcagccaaacctgtggaatttcgaattcctgcaatccctggagaacgacctgaaagtggaaaagctggaggagaaagcgaccaaactggaggaagaagttcgctgcatgatcaaccgtgtagacacccagccgctgtccctgctggagctgatcgacgatgtgcagcgcctgggtctgacctacaaatttgaaaaagacatcattaaagccctggaaaacatcgtactgctggacgaaaacaaaaagaacaaatctgacctgcacgcaaccgctctgtctttccgtctgctgcgtcagcacggtttcgaggtttctcaggatgtttttgagcgtttcaaggataaagaaggtggtttcagcggtgaactgaaaggtgacgtccaaggcctgctgagcctgtatgaagcgtcttacctgggtttcgagggtgagaacctgctggaggaggcgcgtaccttttccatcacccacctgaagaacaacctgaaagaaggcattaataccaaggttgcagaacaagtgagccacgccctggaactgccatatcaccagcgtctgcaccgtctggaggcacgttggttcctggataaatacgaaccgaaagaaccgcatcaccagctgctgctggagctggcgaagctggattttaacatggtacagaccctgcaccagaaagagctgcaagatctgtcccgctggtggaccgagatgggcctggctagcaaactggattttgtacgcgaccgcctgatggaagtttatttctgggcactgggtatggcgccagacccgcagtttggtgaatgtcgcaaagctgttactaaaatgtttggtctggtgacgatcatcgatgacgtgtatgacgtttatggcactctggacgaactgcaactgttcaccgatgctgtagagcgctgggacgttaacgctattaacaccctgccggactatatgaaactgtgtttcctggcactgtacaacaccgttaacgacacgtcctattctattctgaaagagaaaggtcataacaacctgtcctatctgacgaaaagctggcgtgaactgtgcaaagcctttctgcaagaggcgaaatggtccaacaacaaaattatcccggctttctccaagtacctggaaaacgccagcgtttcctcctccggtgtagcgctgctggcgccgtcttacttttccgtatgccagcagcaggaagacatctccgaccacgcgctgcgttccctgaccgacttccatggtctggtgcgttctagctgcgttatcttccgcctgtgcaacgatctggccacctctgcggcggagctggaacgtggcgagactaccaattctatcattagctacatgcacgaaaacgatggtaccagcgaggaacaggcccgcgaagaactgcgtaaactgatcgacgccgaatggaaaaagatgaatcgtgaacgcgttagcgactccaccctgctgcctaaagcgttcatggaaatcgcagttaacatggcacgtgtttcccactgcacctaccagtatggcgatggtctgggtcgcccagactacgcgactgaaaaccgcatcaaactgctgctgattgaccctttcccgattaaccagctgatgtatgtctaactgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggctta

SEQ ID NO: 8 - DNA, 1695 bp, Artificial Sequence
  Synthetic Construct
atgtgtgcaacctcctcccagtttactcagattaccgagcataattctcgacgatctgctaactaccagccgaacctttggaactttgagtttctccagtctctcgaaaatgacctgaaggtggaaaagctcgaggagaaggcgaccaaactcgaggaggaggtgcgatgtatgatcaacagagttgacacccaacccctgtctttgctggagctgatcgacgatgtgcagcggttgggtttgacttataaattcgagaaggacattatcaaggcactggagaacattgtgctcctcgacgagaacaagaagaacaagtctgatcttcacgctaccgctctctctttccgacttcttcgacaacacggcttcgaggtgtcgcaggacgtcttcgagagatttaaggacaaggagggaggatttagcggcgagctgaagggagacgttcagggtcttctctccttgtacgaggcgtcctacctgggattcgagggagagaacctcctggaggaagctcgtacattttccatcactcaccttaagaataaccttaaggagggaattaacaccaaggtggccgagcaggtttctcacgccctggagctcccctaccaccaacggctccatagactggaggctcgttggttcctggacaaatatgagccaaaggagcctcatcatcagttgctgttggagttggccaagctggacttcaatatggttcagacgctgcaccaaaaggagttgcaggacctgtctcgatggtggaccgagatgggattggcctcgaagctggattttgtccgtgaccgacttatggaggtctatttttgggcccttggaatggcgcctgacccccagttcggagagtgccggaaggcggtgacgaagatgttcggtcttgtgactatcatcgacgacgtctacgatgtctacggcacactcgacgagttgcagctgttcactgacgccgtcgagcgatgggatgtgaacgccattaatactctccctgactatatgaagctgtgcttcctggctctgtacaacactgtcaacgatacctcgtactctatcctcaaggagaagggacacaacaatctctcctacttgaccaaatcctggcgagaactgtgcaaggcttttctgcaggaggctaaatggtccaataacaagatcattcctgctttttctaaatacctggaaaatgcctcggtgtcgagctctggcgtcgcccttctggccccttcctacttctccgtctgccagcagcaggaggatatttccgatcatgctcttagatcgctgaccgattttcacggcctcgtgcgatcttcctgcgtgatttttcggttgtgtaatgaccttgcgacctctgctgctgagctggaacgaggcgagactacaaattccattatttcttacatgcacgaaaacgatggaacatctgaagaacaggctagagaggaactgcgaaagttgatcgacgccgagtggaagaagatgaacagagagcgggtgtccgactctaccctgcttcccaaggccttcatggagatcgccgtgaacatggctcgagtttcccattgtacttaccagtacggtgacggcctgggtcgtccggactacgctacagagaaccgaatcaagctgctgctcatcgaccccttccctatcaaccaattgatgtacgtgtaa

SEQ ID NO: 9 - DNA, 40 bp, Artificial Sequence
  Synthetic Construct
gcttatggatcctctagactattacacgtacatcaattgg

SEQ ID NO: 10 - DNA, 30 bp, Artificial Sequence
  Synthetic Construct
caccatgtgtgcaacctcctcccagtttac

SEQ ID NO: 11 - DNA, 8191 bp, Artificial Sequence
  Synthetic Construct
tcgaccggtgagaagaacagcatcgggacaagggaaggaagaacaaagacaaagaaaacaaaagaaagcaattgaaaacaaaacaaaacaattttcattccttctcttatcattccttttcttttcttttctctcattcaacgcactccatcgtatccgtattcctcttattttttctctttctctatatccatttctttctctctaggtgtgtcctctctctctcttcaatttctctactccgcattccaacgcatccttcccccaacctcccatttcctccttacggcccgatagcgatcgtctttccctcgctatcactcgctaccggcccctcctctgcaccgtaacctcctacgtatttaccatatcataaagttttttccgacgcttatcgctgaccccctgtcgccctcctattggcttccggattatcttcttgtccataaggtgatccatgcttcctgaagattcccgaaatgtgtccactttggcggggaatcattccatccacttctttctctctcgctttcctcattcggcgctccccttccgcgtctcattggtcttccgctccgtttttgctttgccgatgttacttggggagaggtgcgataatcctttcgcaaaaactcggtttgacgcctcccatggtataaatagtgggtggtggacaggtgccttcgcttttctttaagcaagagaatcccattgtcttgactatcacgaattcacatacattatgaagatcaccgctgtcattgcccttttattctcacttgctgctgcctcacctattccagttgccgatcctggtgtggtttcagttagcaagtcatatgctgatttccttcgtgtttaccaaagttggaacacttttgctaatcctgatagacccaaccttaagaagagaaatgatacacctgcaagtggatatcaagttgaaaaagtcgtaattttgtcacgtcacggtgttagggcccctacaaaaatgactcaaaccatgcgtgatgtcactcctaatacatggccagaatggcccgttaaattaggatatattacaccaagaggtgaacacttgatatcacttatgggcggtttttaccgtcaaaaattccagcaacaaggaatcctttctcagggctcctgtcctactcctaactccatatatgtctgggctgacgtcgatcagcgtactttaaaaactggtgaagcattccttgctggtttggcaccacaatgtggcttgacaattcatcaccaacaaaatcttgagaaagctgatcctctttttcatcccgttaaagctggaacctgctctatggataaaactcaagttcaacaagctgttgagaaggaggcacaaactcctatagataatttgaatcaacattacatcccctttttagctttaatgaatacaacattaaattttagtacttctgcctggtgccaaaaacactctgctgataaatcctgtgacctaggtttatccatgccttctaaattgtccataaaagataatggtaacaaggtcgcattggatggagctattggtctatcctctactttggccgagatttttcttcttgaatatgctcaaggcatgcctcaagctgcttggggtaacatccactcagagcaagagtgggcttccttgctaaagttgcataatgttcaattcgatttgatggcccgaacaccttatattgctcgacataacggtactcctttattgcaagctatatcaaatgcccttaatcccaacgccactgaatcaaaacttccagatatttcacctgataacaaaatattgttcattgcaggtcatgacacaaatattgctaatatagccggcatgttaaatatgcgttggacattaccaggtcaaccagataatactcctccaggtggtgccctagtatttgaacgtcttgctgataaaagtggaaaacaatatgtttctgtatctatggtttatcaaacactagaacaacttcgatcacagactcccctttctctaaatcagcctgccggatctgttcaacttaaaattccaggttgcaatgatcaaacagccgagggttactgtcctctttccacttttacaagagttgtttcccaatctgttgaacctggatgccaacttcaataatgaggatccaagtaagggaatgagaatgtgatccacttttaattcctaatgaatacatgcctatagttcttttcttttgttctttatgtcgtttttcgatggtacggccgttgtcaatctcagtttgtgtgcttggttgcagcttggtttcaaatctgttcatctcatgaatcttttaccatttcaccacacgtttataccattctctcatagaatcttcatcaaaccatctcggggttagagtggaaagaaagtcttgttcttttatttccttttttccatcttcaaggcttttcttttcttcctcctcctcgttcatcttgaggtttgacgtgtctgtttagaattttgagctgttgcagcatcttattttttgttttgcgaaaacgaagcgctttactctcttcatcagttggacgattgtacctttgaaaaccaactacttttgcatgttttgtatagaaatcaatgatattagaatcccatcctttaatttctttcaaagtagttgagctatagttaagtgtaagggccctactgcgaaagcatttgccaaggatgttttcattaatcaagaacgaaagttaggggatcgaagacgatcagataccgtcgtagtcttaaccataaactatgccgactagggatcgggcaatgtttcatttatcgacttgctcggcaccttacgagaaatcaaagtctttgggttccggggggagtatggtcgcaaggctgaaacttaaaggaattgacggaagggcaccacaatggagtggagcctgcggcttaatttgactcaacacggggaaactcaccaggtccagacatagtaaggattgacagattgagagctctttcttgattctatgggtggtggtgcatggccgttcttagttggtggagtgatttgtctgcttaattgcgataacgaacgagaccttaacctgctaaatagctggatcagccattttggctgatcattagcttcttagagggactattggcataaagccaatggaagtttgaggcaataacaggtctgtgatgcccttagatgttctgggccgcacgcgcgctacactgacggagccaacgagttgaaaaaaatcttttgattttttatccttggccggaaggtctgggtaatcttgttaaactccgtcgtgctggggatagagcattgcaattattgcggccgctcctcaattcgatgttgcagattttacaagtttttaaaatgtatttcattattactttttatatgcctaataaaaaagccatagtttaatctatagataactttttttccagtgcactaacggacgttacattcccatacaaaactgcgtagttaaagctaaggaaaagttaatatcatgttaattaaatacgctatttacaataagacattgaactcatttatatcgttgaatatgaataaccaatttcagcgaatttttaacaaacatcgttcacctcgtttaaggatatcttgtgtatggggtgttgacttgctttatcgaataattaccgtacctgtaattggcttgctggatatagcggtagtctaatatctagcaaaaatcttttgggtgaaaaggcttgcaatttcacgacaccgaactatttgtcattttttaataaggaagttttccataaattcctgtaattctcggttgatctaattgaaaagagtagttttgcatcacgatgaggagggcttttgtagaaagaaatacgaacgaaacgaaaatcagcgttgccatcgctttggacaaagctcccttacctgaagagtcgaattttattgatgaacttataacttccaagcatgcaaaccaaaagggagaacaagtaatccaagtagacacgggaattggattcttggatcacatgtatcatgcactggctaaacatgcaggctggagcttacgactttactcaagaggtgatttaatcatcgatgatcatcacactgcagaagatactgctattgcacttggtattgcattcaagcaggctatgggtaactttgccggcgttaaaagatttggacatgcttattgtccacttgacgaagctctttctagaagcgtagttgacttgtcgggacggccctatgctgttatcgatttgggattaaagcgtgaaaaggttggggaattgtcctgtgaaatgatccctcacttactatattccttttcggtagcagctggaattactttgcatgttacctgcttatatggtagtaatgaccatcatcgtgctgaaagcgcttttaaatctctggctgttgccatgcgcgcggctactagtcttactggaagttctgaagtcccaagcacgaagggagtgttgtaaagatgaattggattatgtcaggaaaagaacgacaattttgcatccaaattgtctaaattttagagttgcttgaaaacaatagaaccttacttgctttataattacgttaattagaagcgttatctcgtgaaggaatatagtacgtagccgtataaattgaattgaatgttcagcttatagaatagagacactttgctgttcaatgcgtcgtcacttaccatactcactttattatacgactttaagtataaactccgcggttatggtaaaattaatgatgcacaaacgtccgattccatatgggtacactacaattaaatacttttaagctgatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaagggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgagcttcccagtaaatgtgccatctcgtaggcagaaaacggttcccccgtagggtctctctcttggcctcctttctaggtcgggctgattgctcttgaagctctctaggggggctcacaccataggcagataacgttccccaccggctcgcctcgtaagcgcacaaggactgctcccaaagatcctaggcgggattttgccgatttcggcctaaaggaaccggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcgccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgatccatggcgatgcctgcttgccgaatatcatggtggaaaatggccgcttttctggattcaacgactgtggccggctgggtgtggcggaccgctatcaggacatagcgttggatacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggtaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgag

SEQ ID NO: 12 - DNA, 1724 bp, Artificial Sequence
  Synthetic Construct
gaattcaaaacaaaatgtgtgcaacctcctcccagtttactcagattaccgagcataattctcgacgatctgctaactaccagccgaacctttggaactttgagtttctccagtctctcgaaaatgacctgaaggtggaaaagctcgaggagaaggcgaccaaactcgaggaggaggtgcgatgtatgatcaacagagttgacacccaacccctgtctttgctggagctgatcgacgatgtgcagcggttgggtttgacttataaattcgagaaggacattatcaaggcactggagaacattgtgctcctcgacgagaacaagaagaacaagtctgatcttcacgctaccgctctctctttccgacttcttcgacaacacggcttcgaggtgtcgcaggacgtcttcgagagatttaaggacaaggagggaggatttagcggcgagctgaagggagacgttcagggtcttctctccttgtacgaggcgtcctacctgggattcgagggagagaacctcctggaggaagctcgtacattttccatcactcaccttaagaataaccttaaggagggaattaacaccaaggtggccgagcaggtttctcacgccctggagctcccctaccaccaacggctccatagactggaggctcgttggttcctggacaaatatgagccaaaggagcctcatcatcagttgctgttggagttggccaagctggacttcaatatggttcagacgctgcaccaaaaggagttgcaggacctgtctcgatggtggaccgagatgggattggcctcgaagctggattttgtccgtgaccgacttatggaggtctatttttgggcccttggaatggcgcctgacccccagttcggagagtgccggaaggcggtgacgaagatgttcggtcttgtgactatcatcgacgacgtctacgatgtctacggcacactcgacgagttgcagctgttcactgacgccgtcgagcgatgggatgtgaacgccattaatactctccctgactatatgaagctgtgcttcctggctctgtacaacactgtcaacgatacctcgtactctatcctcaaggagaagggacacaacaatctctcctacttgaccaaatcctggcgagaactgtgcaaggcttttctgcaggaggctaaatggtccaataacaagatcattcctgctttttctaaatacctggaaaatgcctcggtgtcgagctctggcgtcgcccttctggccccttcctacttctccgtctgccagcagcaggaggatatttccgatcatgctcttagatcgctgaccgattttcacggcctcgtgcgatcttcctgcgtgatttttcggttgtgtaatgaccttgcgacctctgctgctgagctggaacgaggcgagactacaaattccattatttcttacatgcacgaaaacgatggaacatctgaagaacaggctagagaggaactgcgaaagttgatcgacgccgagtggaagaagatgaacagagagcgggtgtccgactctaccctgcttcccaaggccttcatggagatcgccgtgaacatggctcgagtttcccattgtacttaccagtacggtgacggcctgggtcgtccggactacgctacagagaaccgaatcaagctgctgctcatcgaccccttccctatcaaccaattgatgtacgtgtaatagtctagaggatcc

SEQ ID NO: 13 - DNA, 1701 bp, Artificial Sequence
  Synthetic Construct
gaattcaacaaaaatgtgctctgtttccactgagaacgtgtcctttactgagactgagactgaagcacgtagaagcgccaactacgaacccaactcctgggattatgactttctgctgtcttctgacaccgacgagtcgatcgaggtttataaggataaggccaagaaacttgaggccgaggtcagacgagagattaacaacgagaaggccgagttcctgacccttcttgagctgatcgacaacgttcaacgacttggtcttggttaccgtttcgaatccgatatccgacgtgcattggatcgatttgtctcgtccggaggtttcgatggtgtgactaagacgtcgctgcacgccacagctctttccttcagactgttgcggcagcatggatttgaggtttcccaggaagccttttctggtttcaaggatcagaacggaaactttttggagaatctcaaggaggacaccaaggccatcctgtcgttgtatgaggcctcgttcctggctcttgagggcgagaatattctggatgaggctcgggttttcgctatttcgcacctgaaggagttgtcggaggaaaagatcggaaaggaactggccgagcaggtcaaccatgcacttgaacttcccctgcatcgacgtacccagcgactggaggccgtgtggagcatcgaggcgtacagaaaaaaggaggatgctaatcaggttctgctcgaactcgctatcctcgactataacatgattcagagcgtgtaccagcgtgacttgcgagagacaagccggtggtggcgacgggtgggactggccacgaagctccactttgctaaagatcgattgattgagtcgttctactgggcagtgggtgtggcctttgagcctcagtactccgactgccgaaactccgttgcaaagatgttttcttttgtcactatcatcgacgacatctacgatgtttacggcactctcgatgaactcgaactcttcacggacgctgtcgagcgatgggatgtgaatgccattaatgatctgccagattatatgaagttgtgtttcttggcgctctacaacacaattaatgaaattgcctacgacaacctcaaggacaagggagagaacattctgccctaccttactaaagcctgggccgacctgtgtaacgcctttttgcaggaagccaagtggctctataacaaatctactcctacatttgatgactacttcggcaacgcttggaagtcttccagcggccctctccagttgatcttcgcttactttgcagtggtccagaacatcaagaaagaggagattgagaacctccagaagtatcacgacatcatctcccgaccttcgcacatctttcgactgtgcaatgaccttgcctccgcatccgctgagattgcccgaggagaaacagccaattctgtgtcgtgttacatgcgtacaaagggcatctccgaggagctggctaccgagtctgtgatgaacctgatcgatgaaacctgtaagaagatgaacaaagagaaactgggcggttctctgttcgccaaaccatttgttgaaaccgcgatcaatctggctcgtcagtctcattgtacttaccataacggtgacgcgcacacttcgccggacgaattgacccgtaagcgtgtgctttcggtgattaccgagccgatcctgccgttcgaaagataataggatcc

SEQ ID NO: 14 - DNA, 28 bp, Artificial Sequence
  Synthetic Construct
gatcaagcttaaccggaattgccagctg

SEQ ID NO: 15 - DNA, 33 bp, Artificial Sequence
  Synthetic Construct
gatccgatcgtcagaagaactcgtcaagaaggc

SEQ ID NO: 16 - DNA, 38 bp, Artificial Sequence
  Synthetic Construct
catcaatgcatcgcccttaggaggtaaaaaaaaatgac

SEQ ID NO: 17 - DNA, 26 bp, Artificial Sequence
  Synthetic Construct
ccttctgcaggacgcgttgttatagc

SEQ ID NO: 18 - DNA, 60 bp, Artificial Sequence
  Synthetic Construct
gatcatgcattcgcccttaggaggtaaaaaaacatgagttttgatattgccaaatacccg

SEQ ID NO: 19 - DNA, 31 bp, Artificial Sequence
  Synthetic Construct
catgctgcagttatgccagccaggccttgat

SEQ ID NO: 20 - DNA, 8804 bp, Artificial Sequence
  Synthetic Construct
gctggtaccatatgggaattcgaagctttctagaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgcttaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatgtgtgcgacctcttctcaatttactcagattaccgagcataattcccgtcgttccgcaaactatcagccaaacctgtggaatttcgaattcctgcaatccctggagaacgacctgaaagtggaaaagctggaggagaaagcgaccaaactggaggaagaagttcgctgcatgatcaaccgtgtagacacccagccgctgtccctgctggagctgatcgacgatgtgcagcgcctgggtctgacctacaaatttgaaaaagacatcattaaagccctggaaaacatcgtactgctggacgaaaacaaaaagaacaaatctgacctgcacgcaaccgctctgtctttccgtctgctgcgtcagcacggtttcgaggtttctcaggatgtttttgagcgtttcaaggataaagaaggtggtttcagcggtgaactgaaaggtgacgtccaaggcctgctgagcctgtatgaagcgtcttacctgggtttcgagggtgagaacctgctggaggaggcgcgtaccttttccatcacccacctgaagaacaacctgaaagaaggcattaataccaaggttgcagaacaagtgagccacgccctggaactgccatatcaccagcgtctgcaccgtctggaggcacgttggttcctggataaatacgaaccgaaagaaccgcatcaccagctgctgctggagctggcgaagctggattttaacatggtacagaccctgcaccagaaagagctgcaagatctgtcccgctggtggaccgagatgggcctggctagcaaactggattttgtacgcgaccgcctgatggaagtttatttctgggcactgggtatggcgccagacccgcagtttggtgaatgtcgcaaagctgttactaaaatgtttggtctggtgacgatcatcgatgacgtgtatgacgtttatggcactctggacgaactgcaactgttcaccgatgctgtagagcgctgggacgttaacgctattaacaccctgccggactatatgaaactgtgtttcctggcactgtacaacaccgttaacgacacgtcctattctattctgaaagagaaaggtcataacaacctgtcctatctgacgaaaagctggcgtgaactgtgcaaagcctttctgcaagaggcgaaatggtccaacaacaaaattatcccggctttctccaagtacctggaaaacgccagcgtttcctcctccggtgtagcgctgctggcgccgtcttacttttccgtatgccagcagcaggaagacatctccgaccacgcgctgcgttccctgaccgacttccatggtctggtgcgttctagctgcgttatcttccgcctgtgcaacgatctggccacctctgcggcggagctggaacgtggcgagactaccaattctatcattagctacatgcacgaaaacgatggtaccagcgaggaacaggcccgcgaagaactgcgtaaactgatcgacgccgaatggaaaaagatgaatcgtgaacgcgttagcgactccaccctgctgcctaaagcgttcatggaaatcgcagttaacatggcacgtgtttcccactgcacctaccagtatggcgatggtctgggtcgcccagactacgcgactgaaaaccgcatcaaactgctgctgattgaccctttcccgattaaccagctgatgtatgtctaactgcatcgcccttaggaggtaaaaaaaaatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataacaacgcgtcctgcattcgcccttaggaggtaaaaaaacatgagttttgatattgccaaatacccgaccctggcactggtcgactccacccaggagttacgactgttgccgaaagagagtttaccgaaactctgcgacgaactgcgccgctatttactcgacagcgtgagccgttccagcgggcacttcgcctccgggctgggcacggtcgaactgaccgtggcgctgcactatgtctacaacaccccgtttgaccaattgatttgggatgtggggcatcaggcttatccgcataaaattttgaccggacgccgcgacaaaatcggcaccatccgtcagaaaggcggtctgcacccgttcccgtggcgcggcgaaagcgaatatgacgtattaagcgtcgggcattcatcaacctccatcagtgccggaattggtattgcggttgctgccgaaaaagaaggcaaaaatcgccgcaccgtctgtgtcattggcgatggcgcgattaccgcaggcatggcgtttgaagcgatgaatcacgcgggcgatatccgtcctgatatgctggtgattctcaacgacaatgaaatgtcgatttccgaaaatgtcggcgcgctcaacaaccatctggcacagctgctttccggtaagctttactcttcactgcgcgaaggcgggaaaaaagttttctctggcgtgccgccaattaaagagctgctcaaacgcaccgaagaacatattaaaggcatggtagtgcctggcacgttgtttgaagagctgggctttaactacatcggcccggtggacggtcacgatgtgctggggcttatcaccacgctaaagaacatgcgcgacctgaaaggcccgcagttcctgcatatcatgaccaaaaaaggtcgtggttatgaaccggcagaaaaagacccgatcactttccacgccgtgcctaaatttgatccctccagcggttgtttgccgaaaagtagcggcggtttgccgagctattcaaaaatctttggcgactggttgtgcgaaacggcagcgaaagacaacaagctgatggcgattactccggcgatgcgtgaaggttccggcatggtcgagttttcacgtaaattcccggatcgctacttcgacgtggcaattgccgagcaacacgcggtgacctttgctgcgggtctggcgattggtgggtacaaacccattgtcgcgatttactccactttcctgcaacgcgcctatgatcaggtgctgcatgacgtggcgattcaaaagcttccggtcctgttcgccatcgaccgcgcgggcattgttggtgctgacggtcaaacccatcagggtgcttttgatctctcttacctgcgctgcataccggaaatggtcattatgaccccgagcgatgaaaacgaatgtcgccagatgctctataccggctatcactataacgatggcccgtcagcggtgcgctacccgcgtggcaacgcggtcggcgtggaactgacgccgctggaaaaactaccaattggcaaaggcattgtgaagcgtcgtggcgagaaactggcgatccttaactttggtacgctgatgccagaagcggcgaaagtcgccgaatcgctgaacgccacgctggtcgatatgcgttttgtgaaaccgcttgatgaagcgttaattctggaaatggccgccagccatgaagcgctggtcaccgtagaagaaaacgccattatgggcggcgcaggcagcggcgtgaacgaagtgctgatggcccatcgtaaaccagtacccgtgctgaacattggcctgccggacttctttattccgcaaggaactcaggaagaaatgcgcgccgaactcggcctcgatgccgctggtatggaagccaaaatcaaggcctggctggcataactgca

SEQ ID NO: 21 - DNA, 41 bp, Artificial Sequence
  Synthetic Construct
aggaggtaaaaaaacatgtcattaccgttcttaacttctgc

SEQ ID NO: 22 - DNA, 52 bp, Artificial Sequence
  Synthetic Construct
atggctgcaggcctatcgcaaattagcttatgaagtccatggtaaattcgtg

SEQ ID NO: 23 - DNA, 23 bp, Artificial Sequence
  Synthetic Construct
gaattcgcccttctgcagctacc

SEQ ID NO: 24 - DNA, 38 bp, Artificial Sequence
  Synthetic Construct
cgactggtgcacccttaaggaggaaaaaaacatgtcag

SEQ ID NO: 25 - DNA, 25 bp, Artificial Sequence
  Synthetic Construct
gtgctggaattcgcccttctgcagc

SEQ ID NO: 26 - DNA, 32 bp, Artificial Sequence
  Synthetic Construct
gtagatgcatgcagaattcgcccttaaggagg

SEQ ID NO: 27 - DNA, 26 bp, Artificial Sequence
  Synthetic Construct
ccttctgcaggacgcgttgttatagc

SEQ ID NO: 28 - DNA, 38 bp, Artificial Sequence; identical to SEQ ID NO: 16
  Synthetic Construct

SEQ ID NO: 29 - DNA, 25 bp, Artificial Sequence
  Synthetic Construct
gtgtgatggatatctgcagaattcg

SEQ ID NO: 30 - DNA, 36 bp, Artificial Sequence
  Synthetic Construct
catcaatgcatcgcccttaggaggtaaaaaaacatg

SEQ ID NO: 31 - DNA, 60 bp, Artificial Sequence
  Synthetic Construct
gatcatgcattcgcccttaggaggtaaaaaaacatgtgtgcgacctcttctcaatttact

SEQ ID NO: 32 - DNA, 38 bp, Artificial Sequence; identical to SEQ ID NO: 4
  Synthetic Construct

SEQ ID NO: 33 - DNA, 10992 bp, Artificial Sequence
  Synthetic Construct
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggatccgagctcggatccactagtaacggccgccagtgtgctggaattcgcccttaggaggtaaaaaaacatgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcataagctaatttgcgataggcctgcacccttaaggaggaaaaaaacatgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaataacttaaggtagctgcatgcagaattcgcccttaaggaggaaaaaaaaatgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataagatcaattcgctgcatcgcccttaggaggtaaaaaaaaatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataacaacgcgtcctgcattcgcccttaggaggtaaaaaaacatgtgtgcgacctcttctcaatttactcagattaccgagcataattcccgtcgttccgcaaactatcagccaaacctgtggaatttcgaattcctgcaatccctggagaacgacctgaaagtggaaaagctggaggagaaagcgaccaaactggaggaagaagttcgctgcatgatcaaccgtgtagacacccagccgctgtccctgctggagctgatcgacgatgtgcagcgcctgggtctgacctacaaatttgaaaaagacatcattaaagccctggaaaacatcgtactgctggacgaaaacaaaaagaacaaatctgacctgcacgcaaccgctctgtctttccgtctgctgcgtcagcacggtttcgaggtttctcaggatgtttttgagcgtttcaaggataaagaaggtggtttcagcggtgaactgaaaggtgacgtccaaggcctgctgagcctgtatgaagcgtcttacctgggtttcgagggtgagaacctgctggaggaggcgcgtaccttttccatcacccacctgaagaacaacctgaaagaaggcattaataccaaggttgcagaacaagtgagccacgccctggaactgccatatcaccagcgtctgcaccgtctggaggcacgttggttcctggataaatacgaaccgaaagaaccgcatcaccagctgctgctggagctggcgaagctggattttaacatggtacagaccctgcaccagaaagagctgcaagatctgtcccgctggtggaccgagatgggcctggctagcaaactggattttgtacgcgaccgcctgatggaagtttatttctgggcactgggtatggcgccagacccgcagtttggtgaatgtcgcaaagctgttactaaaatgtttggtctggtgacgatcatcgatgacgtgtatgacgtttatggcactctggacgaactgcaactgttcaccgatgctgtagagcgctgggacgttaacgctattaacaccctgccggactatatgaaactgtgtttcctggcactgtacaacaccgttaacgacacgtcctattctattctgaaagagaaaggtcataacaacctgtcctatctgacgaaaagctggcgtgaactgtgcaaagcctttctgcaagaggcgaaatggtccaacaacaaaattatcccggctttctccaagtacctggaaaacgccagcgtttcctcctccggtgtagcgctgctggcgccgtcttacttttccgtatgccagcagcaggaagacatctccgaccacgcgctgcgttccctgaccgacttccatggtctggtgcgttctagctgcgttatcttccgcctgtgcaacgatctggccacctctgcggcggagctggaacgtggcgagactaccaattctatcattagctacatgcacgaaaacgatggtaccagcgaggaacaggcccgcgaagaactgcgtaaactgatcgacgccgaatggaaaaagatgaatcgtgaacgcgttagcgactccaccctgctgcctaaagcgttcatggaaatcgcagttaacatggcacgtgtttcccactgcacctaccagtatggcgatggtctgggtcgcccagactacgcgactgaaaaccgcatcaaactgctgctgattgaccctttcccgattaaccagctgatgtatgtctaactgcagctggtaccatatgggaattcgaagctttctagaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgcttaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 34 - DNA, 50 bp, Artificial Sequence
  Synthetic Construct
gagacatgagctcaggaggtaaaaaaacatgaaaacagtagttattattg

SEQ ID NO: 35 - DNA, 54 bp, Artificial Sequence
  Synthetic Construct
tttatcaatcccaattgtcatgtttttttacctcctttattgttttcttaaatc

SEQ ID NO: 36 - DNA, 54 bp, Artificial Sequence
  Synthetic Construct
gatttaagaaaacaataaaggaggtaaaaaaacatgacaattgggattgataaa

SEQ ID NO: 37 - DNA, 40 bp, Artificial Sequence
  Synthetic Construct
gacatgacatagatctttagtttcgataagaacgaacggt

SEQ ID NO: 38 - DNA, 26 bp, Artificial Sequence
  Synthetic Construct
atgaaaacagtagttattattgatgc

SEQ ID NO: 39 - DNA, 33 bp, Artificial Sequence
  Synthetic Construct
atgttattgttttcttaaatcatttaaaatagc

SEQ ID NO: 40 - DNA, 26 bp, Artificial Sequence
  Synthetic Construct
atgacaattgggattgataaaattag

SEQ ID NO: 41 - DNA, 24 bp, Artificial Sequence
  Synthetic Construct
ttagtttcgataagaacgaacggt

SEQ ID NO: 42 - DNA, 23 bp, Artificial Sequence
  Synthetic Construct
gaaatagccccattagaagtatc

SEQ ID NO: 43 - DNA, 24 bp, Artificial Sequence
  Synthetic Construct
ttgccaatcatatgattgaaaatc

SEQ ID NO: 44 - DNA, 25 bp, Artificial Sequence
  Synthetic Construct
gctatgcttcattagatccttatcg

SEQ ID NO: 45 - DNA, 25 bp, Artificial Sequence
  Synthetic Construct
gaaacctacatccaatcttttgccc

SEQ ID NO: 46 - DNA, 8703 bp, Artificial Sequence
  Synthetic Construct
cccgtcttactgtcgggaattcgcgttggccgattcattaatgcagattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggatccgagctcaggaggtaaaaaaacatgaaaacagtagttattattgatgcattacgaacaccaattggaaaatataaaggcagcttaagtcaagtaagtgccgtagacttaggaacacatgttacaacacaacttttaaaaagacattccactatttctgaagaaattgatcaagtaatctttggaaatgttttacaagctggaaatggccaaaatcccgcacgacaaatagcaataaacagcggtttgtctcatgaaattcccgcaatgacggttaatgaggtctgcggatcaggaatgaaggccgttattttggcgaaacaattgattcaattaggagaagcggaagttttaattgctggcgggattgagaatatgtcccaagcacctaaattacaacgttttaattacgaaacagaaagctacgatgcgcctttttctagtatgatgtatgatggattaacggatgcctttagtggtcaggcaatgggcttaactgctgaaaatgtggccgaaaagtatcatgtaactagagaagagcaagatcaattttctgtacattcacaattaaaagcagctcaagcacaagcagaagggatattcgctgacgaaatagccccattagaagtatcaggaacgcttgtggagaaagatgaagggattcgccctaattcgagcgttgagaagctaggaacgcttaaaacagtttttaaagaagacggtactgtaacagcagggaatgcatcaaccattaatgatggggcttctgctttgattattgcttcacaagaatatgccgaagcacacggtcttccttatttagctattattcgagacagtgtggaagtcggtattgatccagcctatatgggaatttcgccgattaaagccattcaaaaactgttagcgcgcaatcaacttactacggaagaaattgatctgtatgaaatcaacgaagcatttgcagcaacttcaatcgtggtccaaagagaactggctttaccagaggaaaaggtcaacatttatggtggcggtatttcattaggtcatgcgattggtgccacaggtgctcgtttattaacgagtttaagttatcaattaaatcaaaaagaaaagaaatatggagtggcttctttatgtatcggcggtggcttaggactcgctatgctactagagagacctcagcaaaaaaaaaacagccgattttatcaaatgagtcctgaggaacgcctggcttctcttcttaatgaaggccagatttctgctgatacaaaaaaagaatttgaaaatacggctttatcttcgcagattgccaatcatatgattgaaaatcaaatcagtgaaacagaagtgccgatgggcgttggcttacatttaacagtggacgaaactgattatttggtaccaatggcgacagaagagccctcagttattgcggctttgagtaatggtgcaaaaatagcacaaggatttaaaacagtgaatcaacaacgcttaatgcgtggacaaatcgttttttacgatgttgcagatcccgagtcattgattgataaactacaagtaagagaagcggaagtttttcaacaagcagagttaagttatccatctatcgttaaacggggcggcggcttaagagatttgcaatatcgtacttttgatgaatcatttgtatctgtcgactttttagtagatgttaaggatgcaatgggggcaaatatcgttaacgctatgttggaaggtgtggccgagttgttccgtgaatggtttgcggagcaaaagattttattcagtattttaagtaattatgccacggagtcggttgttacgatgaaaacggctattccagtttcacgtttaagtaaggggagcaatggccgggaaattgctgaaaaaattgttttagcttcacgctatgcttcattagatccttatcgggcagtcacgcataacaaaggaatcatgaatggcattgaagctgtagttttagctacaggaaatgatacacgcgctgttagcgcttcttgtcatgcttttgcggtgaaggaaggtcgctaccaaggcttgactagttggacgctggatggcgaacaactaattggtgaaatttcagttccgcttgctttagccacggttggcggtgccacaaaagtcttacctaaatctcaagcagctgctgatttgttagcagtgacggatgcaaaagaactaagtcgagtagtagcggctgttggtttggcacaaaatttagcggcgttacgggccttagtctctgaaggaattcaaaaaggacacatggctctacaagcacgttctttagcgatgacggtcggagctactggtaaagaagttgaggcagtcgctcaacaattaaaacgtcaaaaaacgatgaaccaagaccgagccatggctattttaaatgatttaagaaaacaataaaggaggtaaaaaaacatgacaattgggattgataaaattagtttttttgtgcccccttattatattgatatgacggcactggctgaagccagaaatgtagaccctggaaaatttcatattggtattgggcaagaccaaatggcggtgaacccaatcagccaagatattgtgacatttgcagccaatgccgcagaagcgatcttgaccaaagaagataaagaggccattgatatggtgattgtcgggactgagtccagtatcgatgagtcaaaagcggccgcagttgtcttacatcgtttaatggggattcaacctttcgctcgctctttcgaaatcaaggaagcttgttacggagcaacagcaggcttacagttagctaagaatcacgtagccttacatccagataaaaaagtcttggtcgtagcggcagatattgcaaaatatggcttaaattctggcggtgagcctacacaaggagctggggcggttgcaatgttagttgctagtgaaccgcgcattttggctttaaaagaggataatgtgatgctgacgcaagatatctatgacttttggcgtccaacaggccacccgtatcctatggtcgatggtcctttgtcaaacgaaacctacatccaatcttttgcccaagtctgggatgaacataaaaaacgaaccggtcttgattttgcagattatgatgctttagcgttccatattccttacacaaaaatgggcaaaaaagccttattagcaaaaatctccgaccaaactgaagcagaacaggaacgaattttagcccgttatgaagaaagtatcgtctatagtcgtcgcgtaggaaacttgtatacgggttcactttatctgggactcatttcccttttagaaaatgcaacgactttaaccgcaggcaatcaaattggtttattcagttatggttctggtgctgtcgctgaatttttcactggtgaattagtagctggttatcaaaatcatttacaaaaagaaactcatttagcactgctggataatcggacagaactttctatcgctgaatatgaagccatgtttgcagaaactttagacacagacattgatcaaacgttagaagatgaattaaaatatagtatttctgctattaataataccgttcgttcttatcgaaactaagagatctgcagctggtaccatatgggaattcgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggctta

SEQ ID NO: 47 - DNA, 9371 bp, Artificial Sequence
  Synthetic Construct
tgtaacctttgctttcaaatgagtagaaataatgcacatccatgtttgtatcgtgcaaataaagtgtttcatccgtaggaaaaaatgactttagtatctgttccgctttttctgatgaaatgtgctccccgacaaaattgaatgaatcatggacatttgctggctttgatacagcgaaagcagccgttcctatgttatatatcggatttaacagcaggacaaaaaacaccatgacagccatcgtcacccacttattcacacgcacataaacctttcctgacttttggaacagatgatagctcatcaaaaatcccgccattgccaaataaatcgtatatggcattactgcaccataatcttttgagatttgattgggatatggcgcaagcagcaagacaagcagtccgataatcagcgtataaaataagcctagtaagatcttatccgttctccaatacagcttgaaaaacactacattcaacgcaatgggaagagtgatgatgaaaaacagaaacacgaatgcaatcggctccatcccatccgggtattccttccaatacgaaaagaaactaaaaatcatttgtacgatcggcaaactgacaacagcaaggtcgaacgtataaaacttaccctttccgccatgatcacgcggcatcagcatatagtgaaaagccgtcagcagcacatatccgtataacaaaaaatgcagcagcggcagcagttcttttccgtcctctcttaagtaagcgctggtgaagtttgttgattgcacctggtgaataagttcaacagacactcccgccagcagcacaatccgcaatataacacccgccaagaacattgtgcgctgccggtttattttgggatgatgcaccaaaagatataagcccgccagaacaacaattgaccattgaatcagcagggtgctttgtctgcttaatataaaataacgttcgaaatgcaatacataatgactgaataactccaacacgaacaacaactccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaaagagtgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcataaaaggagagggtgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaataaaaggagagggtgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataaaaggagagggtgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataaaaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatttgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaaaaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataattatcaggatgatgaaaccaactctatattaactttatgccgtatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatttaactataaactatttaaataacagattaaaaaaattataatgtaacctttgctttcaaatgagtagaaataatgcacatccatgtttgtatcgtgcaaataaagtgtttcatccgtaggaaaaaatgactttagtatctgttccgctttttctgatgaaatgtgctccccgacaaaattgaatgaatcatggacatttgctggctttgatacagcgaaagcagccgttcctatgttatatatcggatttaacagcaggacaaaaaacaccatgacagccatcgtcacccacttattcacacgcacataaacctttcctgacttttggaacagatgatagctcatcaaaaatcccgccattgccaaataaatcgtatatggcattactgcaccataatcttttgagatttgattgggatatggcgcaagcagcaagacaagcagtccgataatcagcgtataaaataagcctagtaagatcttatccgttctccaatacagcttgaaaaacactacattcaacgcaatgggaagagtgatgatgaaaaacagaaacacgaatgcaatcggctccatcccatccgggtattccttccaatacgaaaagaaactaaaaatcatttgtacgatcggcaaactgacaacagcaaggtcgaacgtataaaacttaccctttccgccatgatcacgcggcatcagcatatagtgaaaagccgtcagcagcacatatccgtataacaaaaaatgcagcagcggcagcagttcttttccgtcctctcttaagtaagcgctggtgaagtttgttgattgcacctggtgaataagttcaacagacactcccgccagcagcacaatccgcaatataacacccgccaagaacattgtgcgctgccggtttattttgggatgatgcaccaaaagatataagcccgccagaacaacaattgaccattgaatcagcagggtgctttgtctgcttaatataaaataacgttcgaaatgcaatacataatgactgaataactccaacacgaacaacaaaagtgcgcattttataaaagctaatgattcagtccacataattgatagacgaattctgctacaggtcacgtggctatgtgaaggatcgcgcgtccagttaagagcaaaaacattgacaaaaaaatttatttatgctaaaatttactattaatatatttgtatgtataataagattctcctggccaggggaatcttattttttgtggaggatcatttcatgaggaaaaatgagtccagcttaacgtctctaatttcagcttttgcccgtgcatatcacagccgatatgacacacctcttatttttgatgattttatcgcaaaagatctcattaacgaaaaagagtttatcgacatcagtaaaaatatgattcaagaaatatcgtttttcaacaaagagatcgccgaacgtcttcaaaatgatcctgaaaaaatattaaaatgggttgcacaaatccagctgtctccaacgcccctagcacgtgcttcttattgtgaaaaagtcttgcacaacgaattaatcctgggggcaaaacagtatgtcattcttggagcgggactggatactttctgctttcggcatccagaattagaaaacagcttacaggttttcgaggttgatcatccggccacacagcaattgaaaaaaaataagctgaaggatgcaaatctgacaattccgggtcatcttcattttgttcctatggatttcaccaaaacgttttcgtatgatcctctcttagatgaaggatttaaaaacacaaaaacattcttcagccttctcggagtgtcttattatgtaacacgggaagaaaatgcaagcttgatcagcaatttattttctcatgtcccgcctggaagctctattgtttttgattatgcggacgaaacactttttacagcaaaagggacgtcgaatcgagttgaacatatggtgaagatggctgccgcaagcggggaaccgatgaaatcatgtttcacttatcaagagattgaacatctg

SEQ ID NO: 48 - DNA, 4339 bp, Artificial Sequence
  Synthetic Construct
tagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgccctttagtcgctgaacatgtgctctgtttctaccgagaacgtttccttcactgagacggaaaccgaggcacgtcgtagcgcgaactacgagccgaatagctgggactacgatttcctgctgtcttccgatactgacgaatctattgaggtgtacaaagacaaagcaaagaaactggaggctgaagtgcgccgcgaaattaacaacgagaaagctgaattcctgactctgctggagctgatcgataacgtacagcgcctgggtctgggttaccgcttcgaatctgatatccgtcgcgcactggatcgtttcgtaagcagcggcggtttcgatggcgtgaccaaaacgagcctgcacgctaccgcgctgtccttccgtctgctgcgtcagcacggcttcgaagtttctcaggaagcattctccggtttcaaagatcaaaacggtaacttcctggaaaacctgaaagaagacactaaggcgatcctgagcctgtatgaggcaagctttctggccctggagggtgagaacatcctggatgaggcgcgcgtattcgccatctcccatctgaaagagctgtctgaagagaaaatcggtaaggaactggcagagcaggttaatcacgcactggaactgccgctgcatcgtcgtacccagcgtctggaggcggtttggtccatcgaagcgtaccgcaaaaaggaggatgctaaccaggttctgctggaactggccatcctggactacaacatgatccagtccgtttaccagcgtgatctgcgtgaaacctcccgttggtggcgccgtgtgggcctggcgaccaaactgcacttcgctaaggaccgcctgattgagtctttttactgggcagtcggcgttgcgttcgaacctcagtattctgactgccgtaacagcgttgcgaaaatgttcagcttcgttactattatcgacgacatctacgacgtttacggtactctggacgagctggaactgtttaccgacgctgtcgaacgttgggatgttaacgccatcaacgatctgcctgactacatgaaactgtgcttcctggcactgtataacacgatcaacgaaattgcatacgacaacctgaaagacaaaggtgaaaacatcctgccgtacctgactaaagcgtgggcggatctgtgtaacgcttttctgcaagaagcgaaatggctgtataacaaatccactccgacctttgacgattatttcggcaatgcctggaaatccagctctggcccgctgcaactgatcttcgcttattttgcggttgtccaaaacatcaaaaaggaggaaattgaaaacctgcaaaaataccacgatatcattagccgtccttctcatatctttcgcctgtgcaacgacctggcaagcgcgtccgcagagatcgcacgtggcgaaaccgctaactctgtttcctgctacatgcgcaccaagggcatttccgaagagctggcaaccgagagcgtaatgaatctgatcgacgaaacctgtaagaaaatgaacaaagaaaaactgggtggctccctgttcgctaaaccgttcgtagagactgctattaacctggcacgtcagagccactgcacctaccacaatggtgacgcacatactagcccggatgaactgactcgtaaacgtgtactgtctgttatcaccgaaccgattctgccgttcgaacgttaactgcagcgtcaatcgaaagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttt

SEQ ID NO: 49 - DNA, 6065 bp, Artificial Sequence
  Synthetic Construct
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatgtgctctgtttctaccgagaacgtttccttcactgagacggaaaccgaggcacgtcgtagcgcgaactacgagccgaatagctgggactacgatttcctgctgtcttccgatactgacgaatctattgaggtgtacaaagacaaagcaaagaaactggaggctgaagtgcgccgcgaaattaacaacgagaaagctgaattcctgactctgctggagctgatcgataacgtacagcgcctgggtctgggttaccgcttcgaatctgatatccgtcgcgcactggatcgtttcgtaagcagcggcggtttcgatggcgtgaccaaaacgagcctgcacgctaccgcgctgtccttccgtctgctgcgtcagcacggcttcgaagtttctcaggaagcattctccggtttcaaagatcaaaacggtaacttcctggaaaacctgaaagaagacactaaggcgatcctgagcctgtatgaggcaagctttctggccctggagggtgagaacatcctggatgaggcgcgcgtattcgccatctcccatctgaaagagctgtctgaagagaaaatcggtaaggaactggcagagcaggttaatcacgcactggaactgccgctgcatcgtcgtacccagcgtctggaggcggtttggtccatcgaagcgtaccgcaaaaaggaggatgctaaccaggttctgctggaactggccatcctggactacaacatgatccagtccgtttaccagcgtgatctgcgtgaaacctcccgttggtggcgccgtgtgggcctggcgaccaaactgcacttcgctaaggaccgcctgattgagtctttttactgggcagtcggcgttgcgttcgaacctcagtattctgactgccgtaacagcgttgcgaaaatgttcagcttcgttactattatcgacgacatctacgacgtttacggtactctggacgagctggaactgtttaccgacgctgtcgaacgttgggatgttaacgccatcaacgatctgcctgactacatgaaactgtgcttcctggcactgtataacacgatcaacgaaattgcatacgacaacctgaaagacaaaggtgaaaacatcctgccgtacctgactaaagcgtgggcggatctgtgtaacgcttttctgcaagaagcgaaatggctgtataacaaatccactccgacctttgacgattatttcggcaatgcctggaaatccagctctggcccgctgcaactgatcttcgcttattttgcggttgtccaaaacatcaaaaaggaggaaattgaaaacctgcaaaaataccacgatatcattagccgtccttctcatatctttcgcctgtgcaacgacctggcaagcgcgtccgcagagatcgcacgtggcgaaaccgctaactctgtttcctgctacatgcgcaccaagggcatttccgaagagctggcaaccgagagcgtaatgaatctgatcgacgaaacctgtaagaaaatgaacaaagaaaaactgggtggctccctgttcgctaaaccgttcgtagagactgctattaacctggcacgtcagagccactgcacctaccacaatggtgacgcacatactagcccggatgaactgactcgtaaacgtgtactgtctgttatcaccgaaccgattctgccgttcgaacgttaactgcagctggtaccatatgggaattcgaagctttctagaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 50 - DNA, 6912 bp, Artificial Sequence
  Synthetic Construct
ttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatgtgtgcgacctcttctcaatttactcagattaccgagcataattcccgtcgttccgcaaactatcagccaaacctgtggaatttcgaattcctgcaatccctggagaacgacctgaaagtggaaaagctggaggagaaagcgaccaaactggaggaagaagttcgctgcatgatcaaccgtgtagacacccagccgctgtccctgctggagctgatcgacgatgtgcagcgcctgggtctgacctacaaatttgaaaaagacatcattaaagccctggaaaacatcgtactgctggacgaaaacaaaaagaacaaatctgacctgcacgcaaccgctctgtctttccgtctgctgcgtcagcacggtttcgaggtttctcaggatgtttttgagcgtttcaaggataaagaaggtggtttcagcggtgaactgaaaggtgacgtccaaggcctgctgagcctgtatgaagcgtcttacctgggtttcgagggtgagaacctgctggaggaggcgcgtaccttttccatcacccacctgaagaacaacctgaaagaaggcattaataccaaggttgcagaacaagtgagccacgccctggaactgccatatcaccagcgtctgcaccgtctggaggcacgttggttcctggataaatacgaaccgaaagaaccgcatcaccagctgctgctggagctggcgaagctggattttaacatggtacagaccctgcaccagaaagagctgcaagatctgtcccgctggtggaccgagatgggcctggctagcaaactggattttgtacgcgaccgcctgatggaagtttatttctgggcactgggtatggcgccagacccgcagtttggtgaatgtcgcaaagctgttactaaaatgtttggtctggtgacgatcatcgatgacgtgtatgacgtttatggcactctggacgaactgcaactgttcaccgatgctgtagagcgctgggacgttaacgctattaacaccctgccggactatatgaaactgtgtttcctggcactgtacaacaccgttaacgacacgtcctattctattctgaaagagaaaggtcataacaacctgtcctatctgacgaaaagctggcgtgaactgtgcaaagcctttctgcaagaggcgaaatggtccaacaacaaaattatcccggctttctccaagtacctggaaaacgccagcgtttcctcctccggtgtagcgctgctggcgccgtcttacttttccgtatgccagcagcaggaagacatctccgaccacgcgctgcgttccctgaccgacttccatggtctggtgcgttctagctgcgttatcttccgcctgtgcaacgatctggccacctctgcggcggagctggaacgtggcgagactaccaattctatcattagctacatgcacgaaaacgatggtaccagcgaggaacaggcccgcgaagaactgcgtaaactgatcgacgccgaatggaaaaagatgaatcgtgaacgcgttagcgactccaccctgctgcctaaagcgttcatggaaatcgcagttaacatggcacgtgtttcccactgcacctaccagtatggcgatggtctgggtcgcccagactacgcgactgaaaaccgcatcaaactgctgctgattgaccctttcccgattaaccagctgatgtatgtctaactgcatcgcccttaggaggtaaaaaaaaatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataacaacgcgtcctgcagctggtaccatatgggaattcgaagctttctagaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgcttaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggc

SEQ ID NO: 51 - DNA, 7902 bp, Artificial Sequence
  Synthetic Construct
ttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatgtgtgcgacctcttctcaatttactcagattaccgagcataattcccgtcgttccgcaaactatcagccaaacctgtggaatttcgaattcctgcaatccctggagaacgacctgaaagtggaaaagctggaggagaaagcgaccaaactggaggaagaagttcgctgcatgatcaaccgtgtagacacccagccgctgtccctgctggagctgatcgacgatgtgcagcgcctgggtctgacctacaaatttgaaaaagacatcattaaagccctggaaaacatcgtactgctggacgaaaacaaaaagaacaaatctgacctgcacgcaaccgctctgtctttccgtctgctgcgtcagcacggtttcgaggtttctcaggatgtttttgagcgtttcaaggataaagaaggtggtttcagcggtgaactgaaaggtgacgtccaaggcctgctgagcctgtatgaagcgtcttacctgggtttcgagggtgagaacctgctggaggaggcgcgtaccttttccatcacccacctgaagaacaacctgaaagaaggcattaataccaaggttgcagaacaagtgagccacgccctggaactgccatatcaccagcgtctgcaccgtctggaggcacgttggttcctggataaatacgaaccgaaagaaccgcatcaccagctgctgctggagctggcgaagctggattttaacatggtacagaccctgcaccagaaagagctgcaagatctgtcccgctggtggaccgagatgggcctggctagcaaactggattttgtacgcgaccgcctgatggaagtttatttctgggcactgggtatggcgccagacccgcagtttggtgaatgtcgcaaagctgttactaaaatgtttggtctggtgacgatcatcgatgacgtgtatgacgtttatggcactctggacgaactgcaactgttcaccgatgctgtagagcgctgggacgttaacgctattaacaccctgccggactatatgaaactgtgtttcctggcactgtacaacaccgttaacgacacgtcctattctattctgaaagagaaaggtcataacaacctgtcctatctgacgaaaagctggcgtgaactgtgcaaagcctttctgcaagaggcgaaatggtccaacaacaaaattatcccggctttctccaagtacctggaaaacgccagcgtttcctcctccggtgtagcgctgctggcgccgtcttacttttccgtatgccagcagcaggaagacatctccgaccacgcgctgcgttccctgaccgacttccatggtctggtgcgttctagctgcgttatcttccgcctgtgcaacgatctggccacctctgcggcggagctggaacgtggcgagactaccaattctatcattagctacatgcacgaaaacgatggtaccagcgaggaacaggcccgcgaagaactgcgtaaactgatcgacgccgaatggaaaaagatgaatcgtgaacgcgttagcgactccaccctgctgcctaaagcgttcatggaaatcgcagttaacatggcacgtgtttcccactgcacctaccagtatggcgatggtctgggtcgcccagactacgcgactgaaaaccgcatcaaactgctgctgattgaccctttcccgattaaccagctgatgtatgtctaactgcattcgcccttaggaggtaaaaaaacatgagttttgatattgccaaatacccgaccctggcactggtcgactccacccaggagttacgactgttgccgaaagagagtttaccgaaactctgcgacgaactgcgccgctatttactcgacagcgtgagccgttccagcgggcacttcgcctccgggctgggcacggtcgaactgaccgtggcgctgcactatgtctacaacaccccgtttgaccaattgatttgggatgtggggcatcaggcttatccgcataaaattttgaccggacgccgcgacaaaatcggcaccatccgtcagaaaggcggtctgcacccgttcccgtggcgcggcgaaagcgaatatgacgtattaagcgtcgggcattcatcaacctccatcagtgccggaattggtattgcggttgctgccgaaaaagaaggcaaaaatcgccgcaccgtctgtgtcattggcgatggcgcgattaccgcaggcatggcgtttgaagcgatgaatcacgcgggcgatatccgtcctgatatgctggtgattctcaacgacaatgaaatgtcgatttccgaaaatgtcggcgcgctcaacaaccatctggcacagctgctttccggtaagctttactcttcactgcgcgaaggcgggaaaaaagttttctctggcgtgccgccaattaaagagctgctcaaacgcaccgaagaacatattaaaggcatggtagtgcctggcacgttgtttgaagagctgggctttaactacatcggcccggtggacggtcacgatgtgctggggcttatcaccacgctaaagaacatgcgcgacctgaaaggcccgcagttcctgcatatcatgaccaaaaaaggtcgtggttatgaaccggcagaaaaagacccgatcactttccacgccgtgcctaaatttgatccctccagcggttgtttgccgaaaagtagcggcggtttgccgagctattcaaaaatctttggcgactggttgtgcgaaacggcagcgaaagacaacaagctgatggcgattactccggcgatgcgtgaaggttccggcatggtcgagttttcacgtaaattcccggatcgctacttcgacgtggcaattgccgagcaacacgcggtgacctttgctgcgggtctggcgattggtgggtacaaacccattgtcgcgatttactccactttcctgcaacgcgcctatgatcaggtgctgcatgacgtggcgattcaaaagcttccggtcctgttcgccatcgaccgcgcgggcattgttggtgctgacggtcaaacccatcagggtgcttttgatctctcttacctgcgctgcataccggaaatggtcattatgaccccgagcgatgaaaacgaatgtcgccagatgctctataccggctatcactataacgatggcccgtcagcggtgcgctacccgcgtggcaacgcggtcggcgtggaactgacgccgctggaaaaactaccaattggcaaaggcattgtgaagcgtcgtggcgagaaactggcgatccttaactttggtacgctgatgccagaagcggcgaaagtcgccgaatcgctgaacgccacgctggtcgatatgcgttttgtgaaaccgcttgatgaagcgttaattctggaaatggccgccagccatgaagcgctggtcaccgtagaagaaaacgccattatgggcggcgcaggcagcggcgtgaacgaagtgctgatggcccatcgtaaaccagtacccgtgctgaacattggcctgccggacttctttattccgcaaggaactcaggaagaaatgcgcgccgaactcggcctcgatgccgctggtatggaagccaaaatcaaggcctggctggcataactgcagctggtaccatatgggaattcgaagctttctagaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgcttaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggc

SEQ ID NO: 52 - DNA, 6783 bp, Artificial Sequence
  Synthetic Construct
ctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcgggaattcgcgttggccgattcattaatgcagattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatgtgtgcgacctcttctcaatttactcagattaccgagcataattcccgtcgttccgcaaactatcagccaaacctgtggaatttcgaattcctgcaatccctggagaacgacctgaaagtggaaaagctggaggagaaagcgaccaaactggaggaagaagttcgctgcatgatcaaccgtgtagacacccagccgctgtccctgctggagctgatcgacgatgtgcagcgcctgggtctgacctacaaatttgaaaaagacatcattaaagccctggaaaacatcgtactgctggacgaaaacaaaaagaacaaatctgacctgcacgcaaccgctctgtctttccgtctgctgcgtcagcacggtttcgaggtttctcaggatgtttttgagcgtttcaaggataaagaaggtggtttcagcggtgaactgaaaggtgacgtccaaggcctgctgagcctgtatgaagcgtcttacctgggtttcgagggtgagaacctgctggaggaggcgcgtaccttttccatcacccacctgaagaacaacctgaaagaaggcattaataccaaggttgcagaacaagtgagccacgccctggaactgccatatcaccagcgtctgcaccgtctggaggcacgttggttcctggataaatacgaaccgaaagaaccgcatcaccagctgctgctggagctggcgaagctggattttaacatggtacagaccctgcaccagaaagagctgcaagatctgtcccgctggtggaccgagatgggcctggctagcaaactggattttgtacgcgaccgcctgatggaagtttatttctgggcactgggtatggcgccagacccgcagtttggtgaatgtcgcaaagctgttactaaaatgtttggtctggtgacgatcatcgatgacgtgtatgacgtttatggcactctggacgaactgcaactgttcaccgatgctgtagagcgctgggacgttaacgctattaacaccctgccggactatatgaaactgtgtttcctggcactgtacaacaccgttaacgacacgtcctattctattctgaaagagaaaggtcataacaacctgtcctatctgacgaaaagctggcgtgaactgtgcaaagcctttctgcaagaggcgaaatggtccaacaacaaaattatcccggctttctccaagtacctggaaaacgccagcgtttcctcctccggtgtagcgctgctggcgccgtcttacttttccgtatgccagcagcaggaagacatctccgaccacgcgctgcgttccctgaccgacttccatggtctggtgcgttctagctgcgttatcttccgcctgtgcaacgatctggccacctctgcggcggagctggaacgtggcgagactaccaattctatcattagctacatgcacgaaaacgatggtaccagcgaggaacaggcccgcgaagaactgcgtaaactgatcgacgccgaatggaaaaagatgaatcgtgaacgcgttagcgactccaccctgctgcctaaagcgttcatggaaatcgcagttaacatggcacgtgtttcccactgcacctaccagtatggcgatggtctgggtcgcccagactacgcgactgaaaaccgcatcaaactgctgctgattgaccctttcccgattaaccagctgatgtatgtctaactgcagctggtaccatatgggaattcgaagctttctagaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataat

SEQ ID NO: 53 - DNA, 6783 bp, Artificial Sequence
  Synthetic Construct
cccgtcttactgtcgggaattcgcgttggccgattcattaatgcagattattgaagcatttatcagggttattgtctcatgagcggatacatatttgaatgtatttagaaaaataaacaaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagctggagaccgtttaaactcaatgatgatgatgatgatggtcgacggcgctattcagatcctcttctgagatgagtttttgttctagaaagcttcgaattcccatatggtaccagctgcagttagacatacatcagctggttaatcgggaaagggtcaatcagcagcagtttgatgcggttttcagtcgcgtagtctgggcgacccagaccatcgccatactggtaggtgcagtgggaaacacgtgccatgttaactgcgatttccatgaacgctttaggcagcagggtggagtcgctaacgcgttcacgattcatctttttccattcggcgtcgatcagtttacgcagttcttcgcgggcctgttcctcgctggtaccatcgttttcgtgcatgtagctaatgatagaattggtagtctcgccacgttccagctccgccgcagaggtggccagatcgttgcacaggcggaagataacgcagctagaacgcaccagaccatggaagtcggtcagggaacgcagcgcgtggtcggagatgtcttcctgctgctggcatacggaaaagtaagacggcgccagcagcgctacaccggaggaggaaacgctggcgttttccaggtacttggagaaagccgggataattttgttgttggaccatttcgcctcttgcagaaaggctttgcacagttcacgccagcttttcgtcagataggacaggttgttatgacctttctctttcagaatagaataggacgtgtcgttaacggtgttgtacagtgccaggaaacacagtttcatatagtccggcagggtgttaatagcgttaacgtcccagcgctctacagcatcggtgaacagttgcagttcgtccagagtgccataaacgtcatacacgtcatcgatgatcgtcaccagaccaaacattttagtaacagctttgcgacattcaccaaactgcgggtctggcgccatacccagtgcccagaaataaacttccatcaggcggtcgcgtacaaaatccagtttgctagccaggcccatctcggtccaccagcgggacagatcttgcagctctttctggtgcagggtctgtaccatgttaaaatccagcttcgccagctccagcagcagctggtgatgcggttctttcggttcgtatttatccaggaaccaacgtgcctccagacggtgcagacgctggtgatatggcagttccagggcgtggctcacttgttctgcaaccttggtattaatgccttctttcaggttgttcttcaggtgggtgatggaaaaggtacgcgcctcctccagcaggttctcaccctcgaaacccaggtaagacgcttcatacaggctcagcaggccttggacgtcacctttcagttcaccgctgaaaccaccttctttatccttgaaacgctcaaaaacatcctgagaaacctcgaaaccgtgctgacgcagcagacggaaagacagagcggttgcgtgcaggtcagatttgttctttttgttttcgtccagcagtacgatgttttccagggctttaatgatgtctttttcaaatttgtaggtcagacccaggcgctgcacatcgtcgatcagctccagcagggacagcggctgggtgtctacacggttgatcatgcagcgaacttcttcctccagtttggtcgctttctcctccagcttttccactttcaggtcgttctccagggattgcaggaattcgaaattccacaggtttggctgatagtttgcggaacgacgggaattatgctcggtaatctgagtaaattgagaagaggtcgcacacatggtttattcctccttatttaatcgatacattaatatatacctctttaatttttaataataaagttaatcgataattccggtcgagtgcccacacagattgtctgataaattgttaaagagcagtgccgcttcgctttttctcagcggcgctgtttcctgtgtgaaattgttatccgctcacaattccacacattatacgagccggatgattaattgtcaacagctcatttcagaatctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggctta

SEQ ID NO: 54 - DNA, 7687 bp, Artificial Sequence
  Synthetic Construct
ctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcgggaattcgcgttggccgattcattaatgcagattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatgtgtgcgacctcttctcaatttactcagattaccgagcataattcccgtcgttccgcaaactatcagccaaacctgtggaatttcgaattcctgcaatccctggagaacgacctgaaagtggaaaagctggaggagaaagcgaccaaactggaggaagaagttcgctgcatgatcaaccgtgtagacacccagccgctgtccctgctggagctgatcgacgatgtgcagcgcctgggtctgacctacaaatttgaaaaagacatcattaaagccctggaaaacatcgtactgctggacgaaaacaaaaagaacaaatctgacctgcacgcaaccgctctgtctttccgtctgctgcgtcagcacggtttcgaggtttctcaggatgtttttgagcgtttcaaggataaagaaggtggtttcagcggtgaactgaaaggtgacgtccaaggcctgctgagcctgtatgaagcgtcttacctgggtttcgagggtgagaacctgctggaggaggcgcgtaccttttccatcacccacctgaagaacaacctgaaagaaggcattaataccaaggttgcagaacaagtgagccacgccctggaactgccatatcaccagcgtctgcaccgtctggaggcacgttggttcctggataaatacgaaccgaaagaaccgcatcaccagctgctgctggagctggcgaagctggattttaacatggtacagaccctgcaccagaaagagctgcaagatctgtcccgctggtggaccgagatgggcctggctagcaaactggattttgtacgcgaccgcctgatggaagtttatttctgggcactgggtatggcgccagacccgcagtttggtgaatgtcgcaaagctgttactaaaatgtttggtctggtgacgatcatcgatgacgtgtatgacgtttatggcactctggacgaactgcaactgttcaccgatgctgtagagcgctgggacgttaacgctattaacaccctgccggactatatgaaactgtgtttcctggcactgtacaacaccgttaacgacacgtcctattctattctgaaagagaaaggtcataacaacctgtcctatctgacgaaaagctggcgtgaactgtgcaaagcctttctgcaagaggcgaaatggtccaacaacaaaattatcccggctttctccaagtacctggaaaacgccagcgtttcctcctccggtgtagcgctgctggcgccgtcttacttttccgtatgccagcagcaggaagacatctccgaccacgcgctgcgttccctgaccgacttccatggtctggtgcgttctagctgcgttatcttccgcctgtgcaacgatctggccacctctgcggcggagctggaacgtggcgagactaccaattctatcattagctacatgcacgaaaacgatggtaccagcgaggaacaggcccgcgaagaactgcgtaaactgatcgacgccgaatggaaaaagatgaatcgtgaacgcgttagcgactccaccctgctgcctaaagcgttcatggaaatcgcagttaacatggcacgtgtttcccactgcacctaccagtatggcgatggtctgggtcgcccagactacgcgactgaaaaccgcatcaaactgctgctgattgaccctttcccgattaaccagctgatgtatgtctaactgcatcgcccttaggaggtaaaaaaaaatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataacgacgcgtcctgcagctggtaccatatgggaattcgaagctttctagaacgaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataat

SEQ ID NO: 55 - DNA, 8675 bp, Artificial Sequence
  Synthetic Construct
cccgtcttactgtcgggaattcgcgttggccgattcattaatgcagattattgaagcatttatcagggttattgtctcatgagcggatacatatttgaatgtatttagaaaaataaacaaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagctggagaccgtttaaactcaatgatgatgatgatgatggtcgacggcgctattcagatcctcttctgagatgagtttttgttctagaaagcttcgaattcccatatggtaccagctgcagttatgccagccaggccttgattttggcttccataccagcggcatcgaggccgagttcggcgcgcatttcttcctgagttccttgcggaataaagaagtccggcaggccaatgttcagcacgggtactggtttacgatgggccatcagcacttcgttcacgccgctgcctgcgccgcccataatggcgttttcttctacggtgaccagcgcttcatggctggcggccatttccagaattaacgcttcatcaagcggtttcacaaaacgcatatcgaccagcgtggcgttcagcgattcggcgactttcgccgcttctggcatcagcgtaccaaagttaaggatcgccagtttctcgccacgacgcttcacaatgcctttgccaattggtagtttttccagcggcgtcagttccacgccgaccgcgttgccacgcgggtagcgcaccgctgacgggccatcgttatagtgatagccggtatagagcatctggcgacattcgttttcatcgctcggggtcataatgaccatttccggtatgcagcgcaggtaagagagatcaaaagcaccctgatgggtttgaccgtcagcaccaacaatgcccgcgcggtcgatggcgaacaggaccggaagcttttgaatcgccacgtcatgcagcacctgatcataggcgcgttgcaggaaagtggagtaaatcgcgacaatgggtttgtacccaccaatcgccagacccgcagcaaaggtcaccgcgtgttgctcggcaattgccacgtcgaagtagcgatccgggaatttacgtgaaaactcgaccatgccggaaccttcacgcatcgccggagtaatcgccatcagcttgttgtctttcgctgccgtttcgcacaaccagtcgccaaagatttttgaatagctcggcaaaccgccgctacttttcggcaaacaaccgctggagggatcaaatttaggcacggcgtggaaagtgatcgggtctttttctgccggttcataaccacgaccttttttggtcatgatatgcaggaactgcgggcctttcaggtcgcgcatgttctttagcgtggtgataagccccagcacatcgtgaccgtccaccgggccgatgtagttaaagcccagctcttcaaacaacgtgccaggcactaccatgcctttaatatgttcttcggtgcgtttgagcagctctttaattggcggcacgccagagaaaacttttttcccgccttcgcgcagtgaagagtaaagcttaccggaaagcagctgtgccagatggttgttgagcgcgccgacattttcggaaatcgacatttcattgtcgttgagaatcaccagcatatcaggacggatatcgcccgcgtgattcatcgcttcaaacgccatgcctgcggtaatcgcgccatcgccaatgacacagacggtgcggcgatttttgccttctttttcggcagcaaccgcaataccaattccggcactgatggaggttgatgaatgcccgacgcttaatacgtcatattcgctttcgccgcgccacgggaacgggtgcagaccgcctttctgacggatggtgccgattttgtcgcggcgtccggtcaaaattttatgcggataagcctgatgccccacatcccaaatcaattggtcaaacggggtgttgtagacatagtgcagcgccacggtcagttcgaccgtgcccagcccggaggcgaagtgcccgctggaacggctcacgctgtcgagtaaatagcggcgcagttcgtcgcagagtttcggtaaactctctttcggcaacagtcgtaactcctgggtggagtcgaccagtgccagggtcgggtatttggcaatatcaaaactcatgtttttttacctcctaagggcgaatgcagttagacatacatcagctggttaatcgggaaagggtcaatcagcagcagtttgatgcggttttcagtcgcgtagtctgggcgacccagaccatcgccatactggtaggtgcagtgggaaacacgtgccatgttaactgcgatttccatgaacgctttaggcagcagggtggagtcgctaacgcgttcacgattcatctttttccattcggcgtcgatcagtttacgcagttcttcgcgggcctgttcctcgctggtaccatcgttttcgtgcatgtagctaatgatagaattggtagtctcgccacgttccagctccgccgcagaggtggccagatcgttgcacaggcggaagataacgcagctagaacgcaccagaccatggaagtcggtcagggaacgcagcgcgtggtcggagatgtcttcctgctgctggcatacggaaaagtaagacggcgccagcagcgctacaccggaggaggaaacgctggcgttttccaggtacttggagaaagccgggataattttgttgttggaccatttcgcctcttgcagaaaggctttgcacagttcacgccagcttttcgtcagataggacaggttgttatgacctttctctttcagaatagaataggacgtgtcgttaacggtgttgtacagtgccaggaaacacagtttcatatagtccggcagggtgttaatagcgttaacgtcccagcgctctacagcatcggtgaacagttgcagttcgtccagagtgccataaacgtcatacacgtcatcgatgatcgtcaccagaccaaacattttagtaacagctttgcgacattcaccaaactgcgggtctggcgccatacccagtgcccagaaataaacttccatcaggcggtcgcgtacaaaatccagtttgctagccaggcccatctcggtccaccagcgggacagatcttgcagctctttctggtgcagggtctgtaccatgttaaaatccagcttcgccagctccagcagcagctggtgatgcggttctttcggttcgtatttatccaggaaccaacgtgcctccagacggtgcagacgctggtgatatggcagttccagggcgtggctcacttgttctgcaaccttggtattaatgccttctttcaggttgttcttcaggtgggtgatggaaaaggtacgcgcctcctccagcaggttctcaccctcgaaacccaggtaagacgcttcatacaggctcagcaggccttggacgtcacctttcagttcaccgctgaaaccaccttctttatccttgaaacgctcaaaaacatcctgagaaacctcgaaaccgtgctgacgcagcagacggaaagacagagcggttgcgtgcaggtcagatttgttctttttgttttcgtccagcagtacgatgttttccagggctttaatgatgtctttttcaaatttgtaggtcagacccaggcgctgcacatcgtcgatcagctccagcagggacagcggctgggtgtctacacggttgatcatgcagcgaacttcttcctccagtttggtcgctttctcctccagcttttccactttcaggtcgttctccagggattgcaggaattcgaaattccacaggtttggctgatagtttgcggaacgacgggaattatgctcggtaatctgagtaaattgagaagaggtcgcacacatggtttattcctccttatttaatcgatacattaatatatacctctttaatttttaataataaagttaatcgataattccggtcgagtgcccacacagattgtctgataaattgttaaagagcagtgccgcttcgctttttctcagcggcgctgtttcctgtgtgaaattgttatccgctcacaattccacacattatacgagccggatgattaattgtcaacagctcatttcagaatctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggctta

SEQ ID NO: 56 - DNA, 8032 bp, Artificial Sequence
  Synthetic Construct
tcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatagatctggagctgtaatataaaaaccttcttcaactaacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaaagacctatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcactccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaaagagtgaaaacagtagttattattgatgcattacgaacaccaattggaaaatataaaggcagcttaagtcaagtaagtgccgtagacttaggaacacatgttacaacacaacttttaaaaagacattccactatttctgaagaaattgatcaagtaatctttggaaatgttttacaagctggaaatggccaaaatcccgcacgacaaatagcaataaacagcggtttgtctcatgaaattcccgcaatgacggttaatgaggtctgcggatcaggaatgaaggccgttattttggcgaaacaattgattcaattaggagaagcggaagttttaattgctggcgggattgagaatatgtcccaagcacctaaattacaacgttttaattacgaaacagaaagctacgatgcgcctttttctagtatgatgtatgatggattaacggatgcctttagtggtcaggcaatgggcttaactgctgaaaatgtggccgaaaagtatcatgtaactagagaagagcaagatcaattttctgtacattcacaattaaaagcagctcaagcacaagcagaagggatattcgctgacgaaatagccccattagaagtatcaggaacgcttgtggagaaagatgaagggattcgccctaattcgagcgttgagaagctaggaacgcttaaaacagtttttaaagaagacggtactgtaacagcagggaatgcatcaaccattaatgatggggcttctgctttgattattgcttcacaagaatatgccgaagcacacggtcttccttatttagctattattcgagacagtgtggaagtcggtattgatccagcctatatgggaatttcgccgattaaagccattcaaaaactgttagcgcgcaatcaacttactacggaagaaattgatctgtatgaaatcaacgaagcatttgcagcaacttcaatcgtggtccaaagagaactggctttaccagaggaaaaggtcaacatttatggtggcggtatttcattaggtcatgcgattggtgccacaggtgctcgtttattaacgagtttaagttatcaattaaatcaaaaagaaaagaaatatggagtggcttctttatgtatcggcggtggcttaggactcgctatgctactagagagacctcagcaaaaaaaaaacagccgattttatcaaatgagtcctgaggaacgcctggcttctcttcttaatgaaggccagatttctgctgatacaaaaaaagaatttgaaaatacggctttatcttcgcagattgccaatcatatgattgaaaatcaaatcagtgaaacagaagtgccgatgggcgttggcttacatttaacagtggacgaaactgattatttggtaccaatggcgacagaagagccctcagttattgcggctttgagtaatggtgcaaaaatagcacaaggatttaaaacagtgaatcaacaacgcttaatgcgtggacaaatcgttttttacgatgttgcagatcccgagtcattgattgataaactacaagtaagagaagcggaagtttttcaacaagcagagttaagttatccatctatcgttaaacggggcggcggcttaagagatttgcaatatcgtacttttgatgaatcatttgtatctgtcgactttttagtagatgttaaggatgcaatgggggcaaatatcgttaacgctatgttggaaggtgtggccgagttgttccgtgaatggtttgcggagcaaaagattttattcagtattttaagtaattatgccacggagtcggttgttacgatgaaaacggctattccagtttcacgtttaagtaaggggagcaatggccgggaaattgctgaaaaaattgttttagcttcacgctatgcttcattagatccttatcgggcagtcacgcataacaaaggaatcatgaatggcattgaagctgtagttttagctacaggaaatgatacacgcgctgttagcgcttcttgtcatgcttttgcggtgaaggaaggtcgctaccaaggcttgactagttggacgctggatggcgaacaactaattggtgaaatttcagttccgcttgctttagccacggttggcggtgccacaaaagtcttacctaaatctcaagcagctgctgatttgttagcagtgacggatgcaaaagaactaagtcgagtagtagcggctgttggtttggcacaaaatttagcggcgttacgggccttagtctctgaaggaattcaaaaaggacacatggctctacaagcacgttctttagcgatgacggtcggagctactggtaaagaagttgaggcagtcgctcaacaattaaaacgtcaaaaaacgatgaaccaagaccgagccatggctattttaaatgatttaagaaaacaataaaaggagagggtgacaattgggattgataaaattagtttttttgtgcccccttattatattgatatgacggcactggctgaagccagaaatgtagaccctggaaaatttcatattggtattgggcaagaccaaatggcggtgaacccaatcagccaagatattgtgacatttgcagccaatgccgcagaagcgatcttgaccaaagaagataaagaggccattgatatggtgattgtcgggactgagtccagtatcgatgagtcaaaagcggccgcagttgtcttacatcgtttaatggggattcaacctttcgctcgctctttcgaaatcaaggaagcttgttacggagcaacagcaggcttacagttagctaagaatcacgtagccttacatccagataaaaaagtcttggtcgtagcggcagatattgcaaaatatggcttaaattctggcggtgagcctacacaaggagctggggcggttgcaatgttagttgctagtgaaccgcgcattttggctttaaaagaggataatgtgatgctgacgcaagatatctatgacttttggcgtccaacaggccacccgtatcctatggtcgatggtcctttgtcaaacgaaacctacatccaatcttttgcccaagtctgggatgaacataaaaaacgaaccggtcttgattttgcagattatgatgctttagcgttccatattccttacacaaaaatgggcaaaaaagccttattagcaaaaatctccgaccaaactgaagcagaacaggaacgaattttagcccgttatgaagaaagtatcgtctatagtcgtcgcgtaggaaacttgtatacgggttcactttatctgggactcatttcccttttagaaaatgcaacgactttaaccgcaggcaatcaaattggtttattcagttatggttctggtgctgtcgctgaatttttcactggtgaattagtagctggttatcaaaatcatttacaaaaagaaactcatttagcactgctggataatcggacagaactttctatcgctgaatatgaagccatgtttgcagaaactttagacacagacattgatcaaacgttagaagatgaattaaaatatagtatttctgctattaataataccgttcgttcttatcgaaactaaaaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatcgggatccccgggtaccgagctcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgac

SEQ ID NO: 57 - DNA, 6592 bp, Artificial Sequence
  Synthetic Construct
gaattgctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaaagagtgtgtgcgacctcttctcaatttactcagattaccgagcataattcccgtcgttccgcaaactatcagccaaacctgtggaatttcgaattcctgcaatccctggagaacgacctgaaagtggaaaagctggaggagaaagcgaccaaactggaggaagaagttcgctgcatgatcaaccgtgtagacacccagccgctgtccctgctggagctgatcgacgatgtgcagcgcctgggtctgacctacaaatttgaaaaagacatcattaaagccctggaaaacatcgtactgctggacgaaaacaaaaagaacaaatctgacctgcacgcaaccgctctgtctttccgtctgctgcgtcagcacggtttcgaggtttctcaggatgtttttgagcgtttcaaggataaagaaggtggtttcagcggtgaactgaaaggtgacgtccaaggcctgctgagcctgtatgaagcgtcttacctgggtttcgagggtgagaacctgctggaggaggcgcgtaccttttccatcacccacctgaagaacaacctgaaagaaggcattaataccaaggttgcagaacaagtgagccacgccctggaactgccatatcaccagcgtctgcaccgtctggaggcacgttggttcctggataaatacgaaccgaaagaaccgcatcaccagctgctgctggagctggcgaagctggattttaacatggtacagaccctgcaccagaaagagctgcaagatctgtcccgctggtggaccgagatgggcctggctagcaaactggattttgtacgcgaccgcctgatggaagtttatttctgggcactgggtatggcgccagacccgcagtttggtgaatgtcgcaaagctgttactaaaatgtttggtctggtgacgatcatcgatgacgtgtatgacgtttatggcactctggacgaactgcaactgttcaccgatgctgtagagcgctgggacgttaacgctattaacaccctgccggactatatgaaactgtgtttcctggcactgtacaacaccgttaacgacacgtcctattctattctgaaagagaaaggtcataacaacctgtcctatctgacgaaaagctggcgtgaactgtgcaaagcctttctgcaagaggcgaaatggtccaacaacaaaattatcccggctttctccaagtacctggaaaacgccagcgtttcctcctccggtgtagcgctgctggcgccgtcttacttttccgtatgccagcagcaggaagacatctccgaccacgcgctgcgttccctgaccgacttccatggtctggtgcgttctagctgcgttatcttccgcctgtgcaacgatctggccacctctgcggcggagctggaacgtggcgagactaccaattctatcattagctacatgcacgaaaacgatggtaccagcgaggaacaggcccgcgaagaactgcgtaaactgatcgacgccgaatggaaaaagatgaatcgtgaacgcgttagcgactccaccctgctgcctaaagcgttcatggaaatcgcagttaacatggcacgtgtttcccactgcacctaccagtatggcgatggtctgggtcgcccagactacgcgactgaaaaccgcatcaaactgctgctgattgaccctttcccgattaaccagctgatgtatgtctaaaaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatcggatcctctagagtcgacctgcaggcatgcaagctttgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcgaagtcggttcagaaaaagaaggatatggatctggagctgtaatataaaaaccttcttcaactaacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttaaaagaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaagtcaatattactgtaacataaatatatattttaaaaatatcccactttatccaattttcgtttgttgaactaatgggtgctttagttgaagaataaagaccacattaaaaaatgtggtcttttgtgtttttttaaaggatttgagcgtacgcgaaaaatccttttctttctttcttatcttgataataagggtaactattgccggttgtccattcatggctgaactctgcttcctctgttgacatgacacacatcatctcaatatccgaatagggcccatcagtctgacgaccaagagagccataaacaccaatagccttaacatcatccccatatttatccaatattcgttccttaatttcatgaacaatcttcattctttcttctctagtcattattattggtccattcactattctcattcccttttcagataattttagatttgcttttctaaataagaatatttggagagcaccgttcttattcagctattaataactcgtcttcctaagcatccttcaatccttttaataacaattatagcatctaatcttcaacaaactggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgttattctaaaataagttggttccacacataccattacatgcatgtgctgattataagaattatctttattatttattgtcacatccgttgcacgcataaaaccaacaagatttttattaatttttttatattgcatcattcggcgaaatccttgagccatatctgtcaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttgcaaaaccacactcgataccactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttagtcacttataactttaacagatggggtttttctgtgcaaccaattttaagggttttcaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagccgtctgtacgttccttaag

SEQ ID NO: 58 - DNA, 31 bp, Artificial Sequence
  Synthetic Construct
gacatcaattgctccattttcttctgctatc

SEQ ID NO: 59 - DNA, 41 bp, Artificial Sequence
  Synthetic Construct
attgagaagaggtcgcacacactctttaccctctcctttta

SEQ ID NO: 60 - DNA, 41 bp, Artificial Sequence
  Synthetic Construct
taaaaggagagggtaaagagtgtgtgcgacctcttctcaat

SEQ ID NO: 61 - DNA, 41 bp, Artificial Sequence
  Synthetic Construct
ccaaggccggttttttttagacatacatcagctggttaatc

SEQ ID NO: 62 - DNA, 41 bp, Artificial Sequence
  Synthetic Construct
gattaaccagctgatgtatgtctaaaaaaaaccggccttgg

SEQ ID NO: 63 - DNA, 32 bp, Artificial Sequence
  Synthetic Construct
gacatgacggatccgattacgaatgccgtctc

SEQ ID NO: 64 - DNA, 31 bp, Artificial Sequence; identical to SEQ ID NO: 58
  Synthetic Construct

SEQ ID NO: 65 - DNA, 27 bp, Artificial Sequence
  Synthetic Construct
gacatgaattcctccattttcttctgc

SEQ ID NO: 66 - DNA, 20 bp, Artificial Sequence
  Synthetic Construct
aggagagggtaaagagtgag

SEQ ID NO: 67 - DNA, 22 bp, Artificial Sequence
  Synthetic Construct
cttttccatcacccacctgaag

SEQ ID NO: 68 - DNA, 28 bp, Artificial Sequence
  Synthetic Construct
ggcgaaatggtccaacaacaaaattatc

SEQ ID NO: 69 - DNA, 51 bp, Artificial Sequence
  Synthetic Construct
ggtgaattcagtctactggggattcccaaatctatatatactgcaggtgac

SEQ ID NO: 70 - DNA, 23 bp, Artificial Sequence
  Synthetic Construct
gcaggtgggaaactatgcactcc

SEQ ID NO: 71 - DNA, 36 bp, Artificial Sequence
  Synthetic Construct
cctgaattctgttggattggaggattggatagtggg

SEQ ID NO: 72 - DNA, 30 bp, Artificial Sequence
  Synthetic Construct
ggtgtcgacgtacggtcgagcttattgacc

SEQ ID NO: 73 - DNA, 32 bp, Artificial Sequence
  Synthetic Construct
ggtgggcccgcattttgccacctacaagccag

SEQ ID NO: 74 - DNA, 40 bp, Artificial Sequence
  Synthetic Construct
ggtgaattctagaggatcccaacgctgttgcctacaacgg

SEQ ID NO: 75 - DNA, 36 bp, Artificial Sequence
  Synthetic Construct
ggtgcggccgctgtctggacctggtgagtttccccg

SEQ ID NO: 76 - DNA, 37 bp, Artificial Sequence
  Synthetic Construct
ggtgggcccattaaatcagttatcgtttatttgatag

SEQ ID NO: 77 - DNA, 35 bp, Artificial Sequence
  Synthetic Construct
ggtgaccagcaagtccatgggtggtttgatcatgg

SEQ ID NO: 78 - DNA, 35 bp, Artificial Sequence
  Synthetic Construct
ggtgcggccgcctttggagtacgactccaactatg

SEQ ID NO: 79 - DNA, 31 bp, Artificial Sequence
  Synthetic Construct
gcggccgcagactaaatttatttcagtctcc

SEQ ID NO: 80 - DNA, 7 bp, Artificial Sequence
  Synthetic Construct
aggaggt

SEQ ID NO: 81 - DNA, 7 bp, Artificial Sequence
  Synthetic Construct
aaggagg

SEQ ID NO: 82 - DNA, 27 bp, Artificial Sequence
  Synthetic Construct
gacatctgcagctccattttcttctgc

SEQ ID NO: 83 - DNA, 42 bp, Artificial Sequence
  Synthetic Construct
caataataactactgttttcactctttaccctctccttttaa

SEQ ID NO: 84 - DNA, 42 bp, Artificial Sequence
  Synthetic Construct
ttaaaaggagagggtaaagagtgaaaacagtagttattattg

SEQ ID NO: 85 - DNA, 45 bp, Artificial Sequence
  Synthetic Construct
cggggccaaggccggttttttttagtttcgataagaacgaacggt

SEQ ID NO: 86 - DNA, 8000 bp, Artificial Sequence
  Synthetic Construct
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggatccgagctcaggaggtaaaaaaacatgaaaacagtagttattattgatgcattacgaacaccaattggaaaatataaaggcagcttaagtcaagtaagtgccgtagacttaggaacacatgttacaacacaacttttaaaaagacattccactatttctgaagaaattgatcaagtaatctttggaaatgttttacaagctggaaatggccaaaatcccgcacgacaaatagcaataaacagcggtttgtctcatgaaattcccgcaatgacggttaatgaggtctgcggatcaggaatgaaggccgttattttggcgaaacaattgattcaattaggagaagcggaagttttaattgctggcgggattgagaatatgtcccaagcacctaaattacaacgttttaattacgaaacagaaagctacgatgcgcctttttctagtatgatgtatgatggattaacggatgcctttagtggtcaggcaatgggcttaactgctgaaaatgtggccgaaaagtatcatgtaactagagaagagcaagatcaattttctgtacattcacaattaaaagcagctcaagcacaagcagaagggatattcgctgacgaaatagccccattagaagtatcaggaacgcttgtggagaaagatgaagggattcgccctaattcgagcgttgagaagctaggaacgcttaaaacagtttttaaagaagacggtactgtaacagcagggaatgcatcaaccattaatgatggggcttctgctttgattattgcttcacaagaatatgccgaagcacacggtcttccttatttagctattattcgagacagtgtggaagtcggtattgatccagcctatatgggaatttcgccgattaaagccattcaaaaactgttagcgcgcaatcaacttactacggaagaaattgatctgtatgaaatcaacgaagcatttgcagcaacttcaatcgtggtccaaagagaactggctttaccagaggaaaaggtcaacatttatggtggcggtatttcattaggtcatgcgattggtgccacaggtgctcgtttattaacgagtttaagttatcaattaaatcaaaaagaaaagaaatatggagtggcttctttatgtatcggcggtggcttaggactcgctatgctactagagagacctcagcaaaaaaaaaacagccgattttatcaaatgagtcctgaggaacgcctggcttctcttcttaatgaaggccagatttctgctgatacaaaaaaagaatttgaaaatacggctttatcttcgcagattgccaatcatatgattgaaaatcaaatcagtgaaacagaagtgccgatgggcgttggcttacatttaacagtggacgaaactgattatttggtaccaatggcgacagaagagccctcagttattgcggctttgagtaatggtgcaaaaatagcacaaggatttaaaacagtgaatcaacaacgcttaatgcgtggacaaatcgttttttacgatgttgcagatcccgagtcattgattgataaactacaagtaagagaagcggaagtttttcaacaagcagagttaagttatccatctatcgttaaacggggcggcggcttaagagatttgcaatatcgtacttttgatgaatcatttgtatctgtcgactttttagtagatgttaaggatgcaatgggggcaaatatcgttaacgctatgttggaaggtgtggccgagttgttccgtgaatggtttgcggagcaaaagattttattcagtattttaagtaattatgccacggagtcggttgttacgatgaaaacggctattccagtttcacgtttaagtaaggggagcaatggccgggaaattgctgaaaaaattgttttagcttcacgctatgcttcattagatccttatcgggcagtcacgcataacaaaggaatcatgaatggcattgaagctgtagttttagctacaggaaatgatacacgcgctgttagcgcttcttgtcatgcttttgcggtgaaggaaggtcgctaccaaggcttgactagttggacgctggatggcgaacaactaattggtgaaatttcagttccgcttgctttagccacggttggcggtgccacaaaagtcttacctaaatctcaagcagctgctgatttgttagcagtgacggatgcaaaagaactaagtcgagtagtagcggctgttggtttggcacaaaatttagcggcgttacgggccttagtctctgaaggaattcaaaaaggacacatggctctacaagcacgttctttagcgatgacggtcggagctactggtaaagaagttgaggcagtcgctcaacaattaaaacgtcaaaaaacgatgaaccaagaccgagccatggctattttaaatgatttaagaaaacaataaaggaggtaaaaaaacatgacaattgggattgataaaattagtttttttgtgcccccttattatattgatatgacggcactggctgaagccagaaatgtagaccctggaaaatttcatattggtattgggcaagaccaaatggcggtgaacccaatcagccaagatattgtgacatttgcagccaatgccgcagaagcgatcttgaccaaagaagataaagaggccattgatatggtgattgtcgggactgagtccagtatcgatgagtcaaaagcggccgcagttgtcttacatcgtttaatggggattcaacctttcgctcgctctttcgaaatcaaggaagcttgttacggagcaacagcaggcttacagttagctaagaatcacgtagccttacatccagataaaaaagtcttggtcgtagcggcagatattgcaaaatatggcttaaattctggcggtgagcctacacaaggagctggggcggttgcaatgttagttgctagtgaaccgcgcattttggctttaaaagaggataatgtgatgctgacgcaagatatctatgacttttggcgtccaacaggccacccgtatcctatggtcgatggtcctttgtcaaacgaaacctacatccaatcttttgcccaagtctgggatgaacataaaaaacgaaccggtcttgattttgcagattatgatgctttagcgttccatattccttacacaaaaatgggcaaaaaagccttattagcaaaaatctccgaccaaactgaagcagaacaggaacgaattttagcccgttatgaagaaagtatcgtctatagtcgtcgcgtaggaaacttgtatacgggttcactttatctgggactcatttcccttttagaaaatgcaacgactttaaccgcaggcaatcaaattggtttattcagttatggttctggtgctgtcgctgaatttttcactggtgaattagtagctggttatcaaaatcatttacaaaaagaaactcatttagcactgctggataatcggacagaactttctatcgctgaatatgaagccatgtttgcagaaactttagacacagacattgatcaaacgttagaagatgaattaaaatatagtatttctgctattaataataccgttcgttcttatcgaaactaagagatctgcagctggtaccatatgggaattcgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 87 - DNA, 10433 bp, Artificial Sequence
  Synthetic Construct
cccgtcttactgtcgggaattcgcgttggccgattcattaatgcagattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggatccgagctcaggaggtaaaaaaacatgaaaacagtagttattattgatgcattacgaacaccaattggaaaatataaaggcagcttaagtcaagtaagtgccgtagacttaggaacacatgttacaacacaacttttaaaaagacattccactatttctgaagaaattgatcaagtaatctttggaaatgttttacaagctggaaatggccaaaatcccgcacgacaaatagcaataaacagcggtttgtctcatgaaattcccgcaatgacggttaatgaggtctgcggatcaggaatgaaggccgttattttggcgaaacaattgattcaattaggagaagcggaagttttaattgctggcgggattgagaatatgtcccaagcacctaaattacaacgttttaattacgaaacagaaagctacgatgcgcctttttctagtatgatgtatgatggattaacggatgcctttagtggtcaggcaatgggcttaactgctgaaaatgtggccgaaaagtatcatgtaactagagaagagcaagatcaattttctgtacattcacaattaaaagcagctcaagcacaagcagaagggatattcgctgacgaaatagccccattagaagtatcaggaacgcttgtggagaaagatgaagggattcgccctaattcgagcgttgagaagctaggaacgcttaaaacagtttttaaagaagacggtactgtaacagcagggaatgcatcaaccattaatgatggggcttctgctttgattattgcttcacaagaatatgccgaagcacacggtcttccttatttagctattattcgagacagtgtggaagtcggtattgatccagcctatatgggaatttcgccgattaaagccattcaaaaactgttagcgcgcaatcaacttactacggaagaaattgatctgtatgaaatcaacgaagcatttgcagcaacttcaatcgtggtccaaagagaactggctttaccagaggaaaaggtcaacatttatggtggcggtatttcattaggtcatgcgattggtgccacaggtgctcgtttattaacgagtttaagttatcaattaaatcaaaaagaaaagaaatatggagtggcttctttatgtatcggcggtggcttaggactcgctatgctactagagagacctcagcaaaaaaaaaacagccgattttatcaaatgagtcctgaggaacgcctggcttctcttcttaatgaaggccagatttctgctgatacaaaaaaagaatttgaaaatacggctttatcttcgcagattgccaatcatatgattgaaaatcaaatcagtgaaacagaagtgccgatgggcgttggcttacatttaacagtggacgaaactgattatttggtaccaatggcgacagaagagccctcagttattgcggctttgagtaatggtgcaaaaatagcacaaggatttaaaacagtgaatcaacaacgcttaatgcgtggacaaatcgttttttacgatgttgcagatcccgagtcattgattgataaactacaagtaagagaagcggaagtttttcaacaagcagagttaagttatccatctatcgttaaacggggcggcggcttaagagatttgcaatatcgtacttttgatgaatcatttgtatctgtcgactttttagtagatgttaaggatgcaatgggggcaaatatcgttaacgctatgttggaaggtgtggccgagttgttccgtgaatggtttgcggagcaaaagattttattcagtattttaagtaattatgccacggagtcggttgttacgatgaaaacggctattccagtttcacgtttaagtaaggggagcaatggccgggaaattgctgaaaaaattgttttagcttcacgctatgcttcattagatccttatcgggcagtcacgcataacaaaggaatcatgaatggcattgaagctgtagttttagctacaggaaatgatacacgcgctgttagcgcttcttgtcatgcttttgcggtgaaggaaggtcgctaccaaggcttgactagttggacgctggatggcgaacaactaattggtgaaatttcagttccgcttgctttagccacggttggcggtgccacaaaagtcttacctaaatctcaagcagctgctgatttgttagcagtgacggatgcaaaagaactaagtcgagtagtagcggctgttggtttggcacaaaatttagcggcgttacgggccttagtctctgaaggaattcaaaaaggacacatggctctacaagcacgttctttagcgatgacggtcggagctactggtaaagaagttgaggcagtcgctcaacaattaaaacgtcaaaaaacgatgaaccaagaccgagccatggctattttaaatgatttaagaaaacaataaaggaggtaaaaaaacatgacaattgggattgataaaattagtttttttgtgcccccttattatattgatatgacggcactggctgaagccagaaatgtagaccctggaaaatttcatattggtattgggcaagaccaaatggcggtgaacccaatcagccaagatattgtgacatttgcagccaatgccgcagaagcgatcttgaccaaagaagataaagaggccattgatatggtgattgtcgggactgagtccagtatcgatgagtcaaaagcggccgcagttgtcttacatcgtttaatggggattcaacctttcgctcgctctttcgaaatcaaggaagcttgttacggagcaacagcaggcttacagttagctaagaatcacgtagccttacatccagataaaaaagtcttggtcgtagcggcagatattgcaaaatatggcttaaattctggcggtgagcctacacaaggagctggggcggttgcaatgttagttgctagtgaaccgcgcattttggctttaaaagaggataatgtgatgctgacgcaagatatctatgacttttggcgtccaacaggccacccgtatcctatggtcgatggtcctttgtcaaacgaaacctacatccaatcttttgcccaagtctgggatgaacataaaaaacgaaccggtcttgattttgcagattatgatgctttagcgttccatattccttacacaaaaatgggcaaaaaagccttattagcaaaaatctccgaccaaactgaagcagaacaggaacgaattttagcccgttatgaagaaagtatcgtctatagtcgtcgcgtaggaaacttgtatacgggttcactttatctgggactcatttcccttttagaaaatgcaacgactttaaccgcaggcaatcaaattggtttattcagttatggttctggtgctgtcgctgaatttttcactggtgaattagtagctggttatcaaaatcatttacaaaaagaaactcatttagcactgctggataatcggacagaactttctatcgctgaatatgaagccatgtttgcagaaactttagacacagacattgatcaaacgttagaagatgaattaaaatatagtatttctgctattaataataccgttcgttcttatcgaaactaaagatctgcatcctgcattcgcccttaggaggtaaaaaaacatgtgtgcgacctcttctcaatttactcagattaccgagcataattcccgtcgttccgcaaactatcagccaaacctgtggaatttcgaattcctgcaatccctggagaacgacctgaaagtggaaaagctggaggagaaagcgaccaaactggaggaagaagttcgctgcatgatcaaccgtgtagacacccagccgctgtccctgctggagctgatcgacgatgtgcagcgcctgggtctgacctacaaatttgaaaaagacatcattaaagccctggaaaacatcgtactgctggacgaaaacaaaaagaacaaatctgacctgcacgcaaccgctctgtctttccgtctgctgcgtcagcacggtttcgaggtttctcaggatgtttttgagcgtttcaaggataaagaaggtggtttcagcggtgaactgaaaggtgacgtccaaggcctgctgagcctgtatgaagcgtcttacctgggtttcgagggtgagaacctgctggaggaggcgcgtaccttttccatcacccacctgaagaacaacctgaaagaaggcattaataccaaggttgcagaacaagtgagccacgccctggaactgccatatcaccagcgtctgcaccgtctggaggcacgttggttcctggataaatacgaaccgaaagaaccgcatcaccagctgctgctggagctggcgaagctggattttaacatggtacagaccctgcaccagaaagagctgcaagatctgtcccgctggtggaccgagatgggcctggctagcaaactggattttgtacgcgaccgcctgatggaagtttatttctgggcactgggtatggcgccagacccgcagtttggtgaatgtcgcaaagctgttactaaaatgtttggtctggtgacgatcatcgatgacgtgtatgacgtttatggcactctggacgaactgcaactgttcaccgatgctgtagagcgctgggacgttaacgctattaacaccctgccggactatatgaaactgtgtttcctggcactgtacaacaccgttaacgacacgtcctattctattctgaaagagaaaggtcataacaacctgtcctatctgacgaaaagctggcgtgaactgtgcaaagcctttctgcaagaggcgaaatggtccaacaacaaaattatcccggctttctccaagtacctggaaaacgccagcgtttcctcctccggtgtagcgctgctggcgccgtcttacttttccgtatgccagcagcaggaagacatctccgaccacgcgctgcgttccctgaccgacttccatggtctggtgcgttctagctgcgttatcttccgcctgtgcaacgatctggccacctctgcggcggagctggaacgtggcgagactaccaattctatcattagctacatgcacgaaaacgatggtaccagcgaggaacaggcccgcgaagaactgcgtaaactgatcgacgccgaatggaaaaagatgaatcgtgaacgcgttagcgactccaccctgctgcctaaagcgttcatggaaatcgcagttaacatggcacgtgtttcccactgcacctaccagtatggcgatggtctgggtcgcccagactacgcgactgaaaaccgcatcaaactgctgctgattgaccctttcccgattaaccagctgatgtatgtctaactgcagctggtaccatatgggaattcgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggctta

SEQ ID NO: 88 - DNA, 31 bp, Artificial Sequence
  Synthetic Construct
cttgatgcatcctgcattcgcccttaggagg

SEQ ID NO: 89 - DNA, 23 bp, Artificial Sequence
  Synthetic Construct
ccaggcaaattctgttttatcag

SEQ ID NO: 90 - DNA, 10356 bp, Artificial Sequence
  Synthetic Construct
caagaaaaatgccccgcttacgcagggcatccatttattactcaaccgtaaccgattttgccaggttacgcggctggtcaacgtcggtgcctttgatcagcgcgacatggtaagccagcagctgcagcggaacggtgtagaagatcggtgcaatcacctcttccacatgcggcatctcgatgatgtgcatgttatcgctacttacaaaacccgcatcctgatcggcgaagacatacaactgaccgccacgcgcgcgaacttcttcaatgttggatttcagtttttccagcaattcgttgttcggtgcaacaacaataaccggcatatcggcatcaattagcgccagcggaccgtgtttcagttcgccagcagcgtaggcttcagcgtgaatgtaagagatctctttcaacttcaatgcgccttccagcgcgattgggtactgatcgccacggcccaggaacagcgcgtgatgtttgtcagagaaatcttctgccagcgcttcaatgcgtttgtcctgagacagcatctgctcaatacggctcggcagcgcctgcagaccatgcacgatgtcatgttcaatggaggcatccagacctttcaggcgagacagcttcgccaccagcatcaacagcacagttaactgagtggtgaatgctttagtggatgccacgccgatttctgtacccgcgttggtcattagcgccagatcggattcgcgcaccagagaagaacccggaacgttacagattgccagtgaaccaaggtaacccagctctttcgacagacgcaggccagccagggtatccgcggtttcgccagactgtgacacgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgaggagggccaccatggccaagttgaccagtgccgttccggtgctcaccgcgcgcgacgtcgccggagcggtcgagttctggaccgaccggctcgggttctcccctagtaacggccgccagtgtgctggaattcaggcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttcagtgacacaggaacacttaacggctgacagcctgaattctgcagatatctgtttttccactcttcgttcactttcgccaggtagctggtgaagacgaaggaagtcccggagccatctgcgcggcgtactacagcaatgttttgtgaaggcagtttcagacccggattcagtttggcgatggcttcatcatcccacttcttgattttgcccaggtagatgtcgccgagggttttaccatccagcaccagttcgccagacttcagccctggaatgttaaccgccagcaccacgccgccaatcacggtcgggaactggaacagaccttcctgagccagtttttcgtcagacagcggcgcgtcagaggcaccaaaatcaacggtattagcgataatctgttttacgccaccggaagaaccgataccctggtagttaactttattaccggtttctttctggtaagtgtcagcccatttggcatacaccggcgcagggaaggttgcacctgcacctgtcaggcttgcttctgcaaacacagagaaagcactcatcgataaggtcgcggcgacaacagttgcgacggtggtacgcataactttcataatgtctcctgggaggattcataaagcattgtttgttggctacgagaagcaaaataggacaaacaggtgacagttatatgtaaggaatatgacagttttatgacagagagataaagtcttcagtctgatttaaataagcgttgatattcagtcaattacaaacattaataacgaagagatgacagaaaaattttcattctgtgacagagaaaaagtagccgaagatgacggtttgtcacatggagttggcaggatgtttgattaaaagcaattaaccctcactaaagggcggccgcgaagttcctattctctagaaagtataggaacttcattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttccactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcagcacgtgttgacaattaatcatcggcatagtatatcggcatagtataatacgacaaggtgaggaactaaaccatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaagcgggactctggggttcgaataaagaccgaccaagcgacgtctgagagctccctggcgaattcggtaccaataaaagagctttattttcatgatctgtgtgttggtttttgtgtgcggcgcggaagttcctattctctagaaagtataggaacttcctcgagccctatagtgagtcgtattagcccttgacgatgccacatcctgagcaaataattcaaccactaattgtgagcggataacacaaggaggaaacagctatgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcataagctaatttgcgataggcctgcacccttaaggaggaaaaaaacatgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaataacttaaggtagctgcatgcagaattcgcccttaaggaggaaaaaaaaatgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataagatcaattcgctgcatcgcccttaggaggtaaaaaaaaatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataacaacgcgtctacaaataaaaaaggcacgtcagatgacgtgccttttttcttggggcc

SEQ ID NO: 91 - DNA, 43 bp, Artificial Sequence
  Synthetic Construct
gcatgctcgagcggccgcttttaatcaaacatcctgccaactc

SEQ ID NO: 92 - DNA, 37 bp, Artificial Sequence
  Synthetic Construct
gatcgaagggcgatcgtgtcacagtctggcgaaaccg

SEQ ID NO: 93 - DNA, 43 bp, Artificial Sequence
  Synthetic Construct
ctgaattctgcagatatctgtttttccactcttcgttcacttt

SEQ ID NO: 94 - DNA, 33 bp, Artificial Sequence
  Synthetic Construct
tctagagggcccaagaaaaatgccccgcttacg

SEQ ID NO: 95 - DNA, 111 bp, Artificial Sequence
  Synthetic Construct
gatcgcggccgcgcccttgacgatgccacatcctgagcaaataattcaaccactaattgtgagcggataacacaaggaggaaacagctatgtcattaccgttcttaacttc

SEQ ID NO: 96 - DNA, 75 bp, Artificial Sequence
  Synthetic Construct
gatcgggccccaagaaaaaaggcacgtcatctgacgtgccttttttatttgtagacgcgttgttatagcattcta

SEQ ID NO: 97 - DNA, 81 bp, Artificial Sequence
  Synthetic Construct
aaagtagccgaagatgacggtttgtcacatggagttggcaggatgtttgattaaaagcaattaaccctcactaaagggcgg

SEQ ID NO: 98 - DNA, 160 bp, Artificial Sequence
  Synthetic Construct
agagtgttcaccaaaaataataacctttcccggtgcagaagttaagaacggtaatgacatagctgtttcctccttgtgttatccgctcacaattagtggttgaattatttgctcaggatgtggcatcgtcaagggctaatacgactcactatagggctcg

SEQ ID NO: 99 - DNA, 60 bp, Artificial Sequence; identical to SEQ ID NO: 31
  Synthetic Construct

SEQ ID NO: 100 - DNA, 38 bp, Artificial Sequence; identical to SEQ ID NO: 4
  Synthetic Construct

SEQ ID NO: 101 - DNA, 6974 bp, Artificial Sequence
  Synthetic Construct
gtgcggccgcaagcttgtcgacggagctcgaattcggatccctgcagttagacatacatcagctggttaatcgggaaagggtcaatcagcagcagtttgatgcggttttcagtcgcgtagtctgggcgacccagaccatcgccatactggtaggtgcagtgggaaacacgtgccatgttaactgcgatttccatgaacgctttaggcagcagggtggagtcgctaacgcgttcacgattcatctttttccattcggcgtcgatcagtttacgcagttcttcgcgggcctgttcctcgctggtaccatcgttttcgtgcatgtagctaatgatagaattggtagtctcgccacgttccagctccgccgcagaggtggccagatcgttgcacaggcggaagataacgcagctagaacgcaccagaccatggaagtcggtcagggaacgcagcgcgtggtcggagatgtcttcctgctgctggcatacggaaaagtaagacggcgccagcagcgctacaccggaggaggaaacgctggcgttttccaggtacttggagaaagccgggataattttgttgttggaccatttcgcctcttgcagaaaggctttgcacagttcacgccagcttttcgtcagataggacaggttgttatgacctttctctttcagaatagaataggacgtgtcgttaacggtgttgtacagtgccaggaaacacagtttcatatagtccggcagggtgttaatagcgttaacgtcccagcgctctacagcatcggtgaacagttgcagttcgtccagagtgccataaacgtcatacacgtcatcgatgatcgtcaccagaccaaacattttagtaacagctttgcgacattcaccaaactgcgggtctggcgccatacccagtgcccagaaataaacttccatcaggcggtcgcgtacaaaatccagtttgctagccaggcccatctcggtccaccagcgggacagatcttgcagctctttctggtgcagggtctgtaccatgttaaaatccagcttcgccagctccagcagcagctggtgatgcggttctttcggttcgtatttatccaggaaccaacgtgcctccagacggtgcagacgctggtgatatggcagttccagggcgtggctcacttgttctgcaaccttggtattaatgccttctttcaggttgttcttcaggtgggtgatggaaaaggtacgcgcctcctccagcaggttctcaccctcgaaacccaggtaagacgcttcatacaggctcagcaggccttggacgtcacctttcagttcaccgctgaaaccaccttctttatccttgaaacgctcaaaaacatcctgagaaacctcgaaaccgtgctgacgcagcagacggaaagacagagcggttgcgtgcaggtcagatttgttctttttgttttcgtccagcagtacgatgttttccagggctttaatgatgtctttttcaaatttgtaggtcagacccaggcgctgcacatcgtcgatcagctccagcagggacagcggctgggtgtctacacggttgatcatgcagcgaacttcttcctccagtttggtcgctttctcctccagcttttccactttcaggtcgttctccagggattgcaggaattcgaaattccacaggtttggctgatagtttgcggaacgacgggaattatgctcggtaatctgagtaaattgagaagaggtcgcacacatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccaatccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcga

SEQ ID NO: 102 - DNA, 3913 bp, Methanosarcina mazei
gcggccgcgcccttgacgatgccacatcctgagcaaataattcaaccactaattgtgagcggataacacaaggaggaaacagccatggtatcctgttctgcgccgggtaagatttacctgttcggtgaacacgccgtagtttatggcgaaactgcaattgcgtgtgcggtggaactgcgtacccgtgttcgcgcggaactcaatgactctatcactattcagagccagatcggccgcaccggtctggatttcgaaaagcacccttatgtgtctgcggtaattgagaaaatgcgcaaatctattcctattaacggtgttttcttgaccgtcgattccgacatcccggtgggctccggtctgggtagcagcgcagccgttactatcgcgtctattggtgcgctgaacgagctgttcggctttggcctcagcctgcaagaaatcgctaaactgggccacgaaatcgaaattaaagtacagggtgccgcgtccccaaccgatacgtatgtttctaccttcggcggcgtggttaccatcccggaacgtcgcaaactgaaaactccggactgcggcattgtgattggcgataccggcgttttctcctccaccaaagagttagtagctaacgtacgtcagctgcgcgaaagctacccggatttgatcgaaccgctgatgacctctattggcaaaatctctcgtatcggcgaacaactggttctgtctggcgactacgcatccatcggccgcctgatgaacgtcaaccagggtctcctggacgccctgggcgttaacatcttagaactgagccagctgatctattccgctcgtgcggcaggtgcgtttggcgctaaaatcacgggcgctggcggcggtggctgtatggttgcgctgaccgctccggaaaaatgcaaccaagtggcagaagcggtagcaggcgctggcggtaaagtgactatcactaaaccgaccgagcaaggtctgaaagtagattaagccttgacttaatagctgcttatttcgcccttatggtacctagtaggaggaaaaaaacatggaaatgcgtcaaccggctgtcgcaggtcaattctacccactgcgttgcgagaacctggaaaacgaactgaaacgctgcttcgaaggcctggagatccgcgaacaagaagtgctgggcgcagtctgtccgcacgccggttatatgtactctggcaaagttgcggcgcacgtctatgccactctgccggaagctgatacctacgtaatcttcggcccgaaccacaccggctacggtagccctgtctctgtgagccgtgaaacttggaagaccccgttgggcaatatcgatgttgacctggaactggcggacggcttcctgggttccatcgtagatgcggatgaactcggtcacaaatacgaacactctatcgaagttcagctgccgtttctgcaataccgttttgaacgcgatttcaaaattctgccaatctgcatgggtatgcaagacgaagaaaccgcggtcgaagtaggtaacctgctggcggatctgatcagcgagtccggtaaacgtgctgtgatcatcgcaagctctgatttcacccactatgagacggctgaacgtgccaaagaaatcgattccgaagttattgattctatcctgaactttgacatctctggcatgtacgatcgcctgtatcgccgtaacgcctctgtttgcggttacggcccgatcaccgctatgctgacggcaagcaaaaagctgggcggctctcgtgcgactttgctgaaatacgcaaacagcggtgacgtgtccggtgataaagacgctgtggtgggctacgccgccatcatcgttgagtaagctgattaaaggttgaacagataggatttcgtcatggatcctacaaggaggaaaaaaacatgaatgcttctaatgaaccggtgattctgaaactgggtggctctgctattaccgacaaaggtgcctacgaaggcgtagttaaggaagctgatttgctgcgcatcgcacaggaagttagcggtttccgtggcaagatgatcgtggttcatggtgctggtagcttcggccatacgtacgcgaagaaatacggcctggaccgtaccttcgacccagagggcgcaattgttactcatgaatctgttaaaaagctcgcctccaaagttgtaggtgctctgaatagcttcggcgtgcgtgctatcgcggtgcatcctatggactgcgcagtatgccgtaacggtcgtatcgaaacgatgtatctggactccatcaagttaatgctggaaaaaggtctggtgccggttctgcacggcgacgtcgcaatggatattgaactgggcacttgtatcctgtccggtgatcaaatcgttccttacctggccaaagaactgggtatctcccgcctcggcctgggcagcgcagaggatggtgtgctggatatggagggcaaacctgtaccggaaatcaccccagaaactttcgaagagttccgccactgcatcggtggttctggttctactgatgtaaccggtggcatgctgggcaaagtgctggaacttctggaattgagcaaaaattcttccattactagctacattttcaacgctggtaaagcagacaacatctaccgctttctgaatggtgagtccatcggcactcgcatcagcccggacaagcgtgtttaagctagttattaacctaaatgctctaaaccagttatgagctctacaaggaggaaaaaaacatgattaacactaccagccgccgcaaaattgaacacctgaaactctgcgcagaatccccggttgaagcgcgtcaggtatctgccggctttgaagacgttactctgatccaccgcgctttaccggagctgaacatggatgaactggacctcagcgttgatttcctgggtaaacgcatcaaagcgccgttcctgattgcgtctatcacgggtggtcacccagataccatcccggttaacgctgcgctggcagctgctgctgaggagctgggtgttggcatcggcgttggctctcagcgcgcggccattgatgatccgagccaggaagacagcttccgtgtagtgcgtgatgaagccccagatgcgtttgtttatggcaacgtcggcgcagcacagatccgtcagtatggtgttgaaggtgttgaaaaactgatcgaaatgattgacgcagatgccttggcaatccacctgaactttctgcaagaagcggtccaaccggaaggtgaccgcgacgcgaccggttgcctggacatgattaccgaaatttgctctcagattaaaactccggtaatcgtgaaagaaaccggtgcaggcattagccgtgaagatgcgattctgttccagaaagctggcgtgagcgcaatcgacgttggcggcgcgggcggcacctcctgggctggcgtcgaggtctaccgtgctaaagaaagccgtgactctgttagcgagcgtttaggtgagctgttttgggatttcggcattccgacggtagcttctctgattgaatcccgcgtttccttgccgctgatcgcaaccggcggtatccgtaacggtctggacattgctaaaagcattgctctcggcgcaagcgctgccagcgccgctctgccgttcgttggtccgtccctggagggcaaagaatccgttgtacgtgtgctgagctgcatgctggaagaatttaaagcagcaatgtttttgtgcggttgcggcaacatcaaagacctgcacaactctccagtagtggtaactggttggacccgcgaatacctggagcagcgcggttttaacgttaaggacctctccctgccgggcaacgctctgtaagcttcaacgcgtctacaaataaaaaaggcacgtcagatgacgtgccttttttcttgtctaga

SEQ ID NO: 103 - DNA, 6848 bp, Artificial Sequence
  Synthetic Construct
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatgtgtgcgacctcttctcaatttactcagattaccgagcataattcccgtcgttccgcaaactatcagccaaacctgtggaatttcgaattcctgcaatccctggagaacgacctgaaagtggaaaagctggaggagaaagcgaccaaactggaggaagaagttcgctgcatgatcaaccgtgtagacacccagccgctgtccctgctggagctgatcgacgatgtgcagcgcctgggtctgacctacaaatttgaaaaagacatcattaaagccctggaaaacatcgtactgctggacgaaaacaaaaagaacaaatctgacctgcacgcaaccgctctgtctttccgtctgctgcgtcagcacggtttcgaggtttctcaggatgtttttgagcgtttcaaggataaagaaggtggtttcagcggtgaactgaaaggtgacgtccaaggcctgctgagcctgtatgaagcgtcttacctgggtttcgagggtgagaacctgctggaggaggcgcgtaccttttccatcacccacctgaagaacaacctgaaagaaggcattaataccaaggttgcagaacaagtgagccacgccctggaactgccatatcaccagcgtctgcaccgtctggaggcacgttggttcctggataaatacgaaccgaaagaaccgcatcaccagctgctgctggagctggcgaagctggattttaacatggtacagaccctgcaccagaaagagctgcaagatctgtcccgctggtggaccgagatgggcctggctagcaaactggattttgtacgcgaccgcctgatggaagtttatttctgggcactgggtatggcgccagacccgcagtttggtgaatgtcgcaaagctgttactaaaatgtttggtctggtgacgatcatcgatgacgtgtatgacgtttatggcactctggacgaactgcaactgttcaccgatgctgtagagcgctgggacgttaacgctattaacaccctgccggactatatgaaactgtgtttcctggcactgtacaacaccgttaacgacacgtcctattctattctgaaagagaaaggtcataacaacctgtcctatctgacgaaaagctggcgtgaactgtgcaaagcctttctgcaagaggcgaaatggtccaacaacaaaattatcccggctttctccaagtacctggaaaacgccagcgtttcctcctccggtgtagcgctgctggcgccgtcttacttttccgtatgccagcagcaggaagacatctccgaccacgcgctgcgttccctgaccgacttccatggtctggtgcgttctagctgcgttatcttccgcctgtgcaacgatctggccacctctgcggcggagctggaacgtggcgagactaccaattctatcattagctacatgcacgaaaacgatggtaccagcgaggaacaggcccgcgaagaactgcgtaaactgatcgacgccgaatggaaaaagatgaatcgtgaacgcgttagcgactccaccctgctgcctaaagcgttcatggaaatcgcagttaacatggcacgtgtttcccactgcacctaccagtatggcgatggtctgggtcgcccagactacgcgactgaaaaccgcatcaaactgctgctgattgaccctttcccgattaaccagctgatgtatgtctaactgcataaaggaggtaaaaaaacatggtatcctgttctgcgccgggtaagatttacctgttcggtgaacacgccgtagtttatggcgaaactgcaattgcgtgtgcggtggaactgcgtacccgtgttcgcgcggaactcaatgactctatcactattcagagccagatcggccgcaccggtctggatttcgaaaagcacccttatgtgtctgcggtaattgagaaaatgcgcaaatctattcctattaacggtgttttcttgaccgtcgattccgacatcccggtgggctccggtctgggtagcagcgcagccgttactatcgcgtctattggtgcgctgaacgagctgttcggctttggcctcagcctgcaagaaatcgctaaactgggccacgaaatcgaaattaaagtacagggtgccgcgtccccaaccgatacgtatgtttctaccttcggcggcgtggttaccatcccggaacgtcgcaaactgaaaactccggactgcggcattgtgattggcgataccggcgttttctcctccaccaaagagttagtagctaacgtacgtcagctgcgcgaaagctacccggatttgatcgaaccgctgatgacctctattggcaaaatctctcgtatcggcgaacaactggttctgtctggcgactacgcatccatcggccgcctgatgaacgtcaaccagggtctcctggacgccctgggcgttaacatcttagaactgagccagctgatctattccgctcgtgcggcaggtgcgtttggcgctaaaatcacgggcgctggcggcggtggctgtatggttgcgctgaccgctccggaaaaatgcaaccaagtggcagaagcggtagcaggcgctggcggtaaagtgactatcactaaaccgaccgagcaaggtctgaaagtagattaaagtctagttaaagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgcttaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 104 - DNA, 6647 bp, Artificial Sequence
  Synthetic Construct
aagggcgagctcaacgatccggctgctaacaaagcccgaaaggaagctgagttggctgctgccaccgctgagcaataactagcataaccccttggggcctctaaacgggtcttgaggagttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgattgaacaagatggattgcacgcaggttctccggccgcttgggtggagaggctattcggctatgactgggcacaactgacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgggcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgcggggttctcatcatcatcatcatcatggtatggctagcatgactggtggacagcaaatgggtcgggatctgtacgacgatgacgataaggatcatcccttcaccatggtatcctgttctgcgccgggtaagatttacctgttcggtgaacacgccgtagtttatggcgaaactgcaattgcgtgtgcggtggaactgcgtacccgtgttcgcgcggaactcaatgactctatcactattcagagccagatcggccgcaccggtctggatttcgaaaagcacccttatgtgtctgcggtaattgagaaaatgcgcaaatctattcctattaacggtgttttcttgaccgtcgattccgacatcccggtgggctccggtctgggtagcagcgcagccgttactatcgcgtctattggtgcgctgaacgagctgttcggctttggcctcagcctgcaagaaatcgctaaactgggccacgaaatcgaaattaaagtacagggtgccgcgtccccaaccgatacgtatgtttctaccttcggcggcgtggttaccatcccggaacgtcgcaaactgaaaactccggactgcggcattgtgattggcgataccggcgttttctcctccaccaaagagttagtagctaacgtacgtcagctgcgcgaaagctacccggatttgatcgaaccgctgatgacctctattggcaaaatctctcgtatcggcgaacaactggttctgtctggcgactacgcatccatcggccgcctgatgaacgtcaaccagggtctcctggacgccctgggcgttaacatcttagaactgagccagctgatctattccgctcgtgcggcaggtgcgtttggcgctaaaatcacgggcgctggcggcggtggctgtatggttgcgctgaccgctccggaaaaatgcaaccaagtggcagaagcggtagcaggcgctggcggtaaagtgactatcactaaaccgaccgagcaaggtctgaaagtagattaa

SEQ ID NO: 105 - DNA, 4608 bp, Streptomyces
gcggccgcgcccttgacgatgccacatcctgagcaaataattcaaccactaattgtgagcggataacacaaggaggaaacagcatgcaaaaacgccaacgtgaactttccgcgcttactttgccaacctctgcggaaggtgtatccgaaagccaccgtgcccgttccgtaggcatcggccgtgcacatgctaaagcaatcctgttgggcgaacacgctgtagtatacggcgctccagctctggcactccctatcccgcaactgaccgttacggcgagcgttggctggtccagcgaggcttcggatagcgctggtggtctgtcttacactatgactggcaccccgtctcgtgcgcttgtgacccaggcaagcgacggtctgcaccgtctgactgctgaatttatggcacgtatgggcgtgaccaatgctccgcacctggatgttatcctggatggtgctatcccgcacggtcgtggcctgggtagctctgctgccggttcccgcgctatcgctctggcgctggcggacctgttcggtcacgaactggctgagcacaccgcgtatgaactcgtccagacggctgagaacatggcacacggccgtgcttctggcgtagacgcaatgacggtaggtgcaagccgtccacttctgttccaacagggccgtaccgaacgtctggcgatcggttgtgactccctgttcatcgtcgcggattccggtgtgccgggtagcaccaaggaagcggtcgaaatgctgcgtgaaggtttcacgcgcagcgcaggcacccaggagcgtttcgtaggccgtgccaccgagctgaccgaggcagcgcgtcaagcactggctgatggccgtccggaggagctgggctcccagctgacttactaccacgaactgcttcacgaagcccgcctgagcaccgacggcatcgacgccctggtggaagcggctttgaaggccggcagcctgggcgcgaaaatcaccggcggtggcctgggcggttgtatgattgctcaggcccgtcctgaacaggcccgtgaagttacccgccaactgcacgaggccggtgcagtacagacctgggttgttccgctgaaaggccttgataaccatgcgcagtaagccttcttaaggtagctgctttcgcccttataagctttacaaggaggaaaaaaacatgactaccggccaacgtactatcgtccgccatgcgccaggtaaactctttgtcgcaggtgaatacgcggtggtcgatccgggtaaccctgcgattctggtagcggtggaccgccacatctccgttaccgtgagcgacgcagatgcagacaccggtgcagcagatgtcgttatcagcagcgaccttggtccgcaggcggtaggctggcgttggcacgacggccgtctggttgttcgtgacccggatgacggtcaacaggcacgttccgccctggcacatgtcgttagcgctattgagactgttggtcgtctgctgggtgaacgcggtcaaaaagtgccggctctgaccctctccgtatcctcccgtcttcacgaagatggccgcaaattcggtctgggttcctctggtgccgttaccgtagcgacggtcgcagctgttgctgcattttgcggtctggaactgtccacggatgaacgctttcgcctggcgatgctggcaaccgctgaactggacccaaaaggttctggcggtgatctggcggcgtctacctggggtggttggatcgcgtatcaggcgccggaccgcgccttcgtactggacctggcgcgtcgtgtgggtgtggaccgtacgctgaaagcgccgtggccgggtcactccgtgcgtcgcctgccggcaccgaagggcctgaccctggaagttggctggactggcgaaccagcaagcaccgcatctctggtttctgatctgcaccgtcgtacctggcgcggttccgcatctcatcagcgcttcgttgagaccaccaccgattgtgtacgctctgctgtaactgcacttgagtccggtgatgacacctctttgctgcacgaaatccgtcgcgctcgccaggaactggctcgtctggacgacgaagtaggcctgggcatcttcactccgaaactcactgccctgtgtgatgcggcggaagcggtaggtggtgccgcaaagccgtctggtgcaggcggtggcgactgtggcattgccctgctcgacgccgaagcgagccgtgatattacccacgttcgccagcgttgggaaaccgctggtgttcttccgctgccgctcacgccggctctcgagggcatctaagcctgacttaaggtagctgcttcccttatctgcagacaaggaggaaaaaacatgcgttctgaacatccaactactaccgttttgcagtcgcgtgaacagggctccgcggcaggtgcgaccgcagttgctcatccgaatatcgctctgatcaaatactggggtaaacgtgatgagcgtctgatcctgccatgcactacttccctgagcatgactctcgatgtattcccgacgaccaccgaagttcgcctggacccggcagctgaacacgatacggctgctcttaacggtgaagtggcaactggtgaaaccctccgtcgtatctctgcattcctgagcttggttcgtgaggtggctggcagcgaccagcgtgcggtagtcgatacccgtaatactgttcctacgggtgcgggtctggcatcctcagcgtcgggtttcgctgccctggctgtagccgcggcagccgcgtacggcctggaactggatgaccgtggcctgagccgtctcgcacgtcgtggttccggttccgcctctcgtagcattttcggcggtttcgcggtttggcacgcaggcccggacggcaccgccactgaagcagacctgggttcttatgcggagcctgttccggcagcggatctggacccagcgctggtgatcgctgtagtaaacgctggcccgaaaccagtttcctctcgcgaagcgatgcgccgtaccgttgatacttccccgctgtatcgtccgtgggccgattcttccaaagatgacctggacgaaatgcgtagcgcactgctccgtggcgatctggaagccgtgggtgagattgcagaacgtaatgctctgggtatgcacgctactatgctcgctgcacgtccggcggtccgttatctgtccccggctaccgttaccgttctggattctgttctgcaactgcgtaaagatggtgtgctcgcatacgcgacgatggatgcgggcccgaacgtgaaggttctgtgccgtcgtgcagatgctgagcgtgttgctgatgttgtacgtgcagctgcgtctggcggtcaggttctggttgcaggcccaggtgatggtgcgcgtctgctgtctgaaggtgcctaacgttcataaggtagctgcaatcgggttatggatcctacaaggaggaaaaaaacatgacttctgctcagcgtaaagatgaccatgttcgtttggcaatcgaacagcacaacgcccattccggccgcaaccagtttgatgacgtatcttttgttcaccacgcactggcaggcattgatcgcccggacgtcagcctggctacttctttcgccggtatctcttggcaggttccgatctacatcaacgcaatgactggtggttccgagaaaaccggcctgatcaaccgtgacctggctactgcggcgcgtgaaactggcgttccgatcgcgtccggttctatgaacgcttacattaaggacccgagctgcgccgatactttccgcgtgctgcgcgatgagaatccgaacggttttgtaatcgcgaacatcaacgctactaccactgttgacaacgctcagcgtgctattgacctgatcgaggcgaacgccctgcaaatccacattaacacggcacaggagacgcctatgccggaaggcgaccgttctttcgcgtcttgggtgcctcagatcgaaaagattgctgcggcagtggacatcccggttatcgtgaaagaagttggtaacggcctgtctcgtcagaccattctcctgctggcggatcttggtgtgcaggcagcggatgttagcggccgtggcggtactgacttcgcacgtatcgaaaacggccgtcgtgaactcggcgactatgcgtttctgcacggctggggtcagtcaaccgcagcgtgtctgctggacgcccaagatatttccctgccggtgctcgcgtctggtggtgtacgccacccactggacgttgtacgcgcgctggcactgggtgcgcgtgcggtcggttcctccgcgggtttcctgcgtaccctgatggatgatggcgttgatgctctgattaccaaactcaccacctggctggatcaactggctgcgctgcaaaccatgctgggcgctcgcaccccagcggacttgacccgttgcgatgttctgctgcacggtgaactgcgtgacttctgcgcggaccgcggcatcgatactcgtcgtctggctcagcgttcctccagcatcgaagcgctccagacgaccggctctacccgttaacaacgcgtctacaaataaaaaaggcacgtcagatgacgtgccttttttcttgtctaga

SEQ ID NO: 106 - DNA, 7280 bp, Artificial Sequence
  Synthetic Construct
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatgtgtgcgacctcttctcaatttactcagattaccgagcataattcccgtcgttccgcaaactatcagccaaacctgtggaatttcgaattcctgcaatccctggagaacgacctgaaagtggaaaagctggaggagaaagcgaccaaactggaggaagaagttcgctgcatgatcaaccgtgtagacacccagccgctgtccctgctggagctgatcgacgatgtgcagcgcctgggtctgacctacaaatttgaaaaagacatcattaaagccctggaaaacatcgtactgctggacgaaaacaaaaagaacaaatctgacctgcacgcaaccgctctgtctttccgtctgctgcgtcagcacggtttcgaggtttctcaggatgtttttgagcgtttcaaggataaagaaggtggtttcagcggtgaactgaaaggtgacgtccaaggcctgctgagcctgtatgaagcgtcttacctgggtttcgagggtgagaacctgctggaggaggcgcgtaccttttccatcacccacctgaagaacaacctgaaagaaggcattaataccaaggttgcagaacaagtgagccacgccctggaactgccatatcaccagcgtctgcaccgtctggaggcacgttggttcctggataaatacgaaccgaaagaaccgcatcaccagctgctgctggagctggcgaagctggattttaacatggtacagaccctgcaccagaaagagctgcaagatctgtcccgctggtggaccgagatgggcctggctagcaaactggattttgtacgcgaccgcctgatggaagtttatttctgggcactgggtatggcgccagacccgcagtttggtgaatgtcgcaaagctgttactaaaatgtttggtctggtgacgatcatcgatgacgtgtatgacgtttatggcactctggacgaactgcaactgttcaccgatgctgtagagcgctgggacgttaacgctattaacaccctgccggactatatgaaactgtgtttcctggcactgtacaacaccgttaacgacacgtcctattctattctgaaagagaaaggtcataacaacctgtcctatctgacgaaaagctggcgtgaactgtgcaaagcctttctgcaagaggcgaaatggtccaacaacaaaattatcccggctttctccaagtacctggaaaacgccagcgtttcctcctccggtgtagcgctgctggcgccgtcttacttttccgtatgccagcagcaggaagacatctccgaccacgcgctgcgttccctgaccgacttccatggtctggtgcgttctagctgcgttatcttccgcctgtgcaacgatctggccacctctgcggcggagctggaacgtggcgagactaccaattctatcattagctacatgcacgaaaacgatggtaccagcgaggaacaggcccgcgaagaactgcgtaaactgatcgacgccgaatggaaaaagatgaatcgtgaacgcgttagcgactccaccctgctgcctaaagcgttcatggaaatcgcagttaacatggcacgtgtttcccactgcacctaccagtatggcgatggtctgggtcgcccagactacgcgactgaaaaccgcatcaaactgctgctgattgaccctttcccgattaaccagctgatgtatgtctaactgcataaaggaggtaaaaaaacatgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcataactgcagagtctagttaaagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgcttaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 107 - DNA, 6896 bp, Artificial Sequence
  Synthetic Construct
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatgtgtgcgacctcttctcaatttactcagattaccgagcataattcccgtcgttccgcaaactatcagccaaacctgtggaatttcgaattcctgcaatccctggagaacgacctgaaagtggaaaagctggaggagaaagcgaccaaactggaggaagaagttcgctgcatgatcaaccgtgtagacacccagccgctgtccctgctggagctgatcgacgatgtgcagcgcctgggtctgacctacaaatttgaaaaagacatcattaaagccctggaaaacatcgtactgctggacgaaaacaaaaagaacaaatctgacctgcacgcaaccgctctgtctttccgtctgctgcgtcagcacggtttcgaggtttctcaggatgtttttgagcgtttcaaggataaagaaggtggtttcagcggtgaactgaaaggtgacgtccaaggcctgctgagcctgtatgaagcgtcttacctgggtttcgagggtgagaacctgctggaggaggcgcgtaccttttccatcacccacctgaagaacaacctgaaagaaggcattaataccaaggttgcagaacaagtgagccacgccctggaactgccatatcaccagcgtctgcaccgtctggaggcacgttggttcctggataaatacgaaccgaaagaaccgcatcaccagctgctgctggagctggcgaagctggattttaacatggtacagaccctgcaccagaaagagctgcaagatctgtcccgctggtggaccgagatgggcctggctagcaaactggattttgtacgcgaccgcctgatggaagtttatttctgggcactgggtatggcgccagacccgcagtttggtgaatgtcgcaaagctgttactaaaatgtttggtctggtgacgatcatcgatgacgtgtatgacgtttatggcactctggacgaactgcaactgttcaccgatgctgtagagcgctgggacgttaacgctattaacaccctgccggactatatgaaactgtgtttcctggcactgtacaacaccgttaacgacacgtcctattctattctgaaagagaaaggtcataacaacctgtcctatctgacgaaaagctggcgtgaactgtgcaaagcctttctgcaagaggcgaaatggtccaacaacaaaattatcccggctttctccaagtacctggaaaacgccagcgtttcctcctccggtgtagcgctgctggcgccgtcttacttttccgtatgccagcagcaggaagacatctccgaccacgcgctgcgttccctgaccgacttccatggtctggtgcgttctagctgcgttatcttccgcctgtgcaacgatctggccacctctgcggcggagctggaacgtggcgagactaccaattctatcattagctacatgcacgaaaacgatggtaccagcgaggaacaggcccgcgaagaactgcgtaaactgatcgacgccgaatggaaaaagatgaatcgtgaacgcgttagcgactccaccctgctgcctaaagcgttcatggaaatcgcagttaacatggcacgtgtttcccactgcacctaccagtatggcgatggtctgggtcgcccagactacgcgactgaaaaccgcatcaaactgctgctgattgaccctttcccgattaaccagctgatgtatgtctaactgcataaaggaggtaaaaaaacatgcaaacgagtgtgggaaacagtcacgctaagattattttgattggagagcatgctgtcgtgtacgggcaacccgcgattgcgcttcctatttttgcagtccagacaacggttcatgtcacggcacaaccaacactgagcgaacccttgattaacagcccttattacagtggccctttaagtcaagcgattggtcagatgcaaggtattcgtcgattaatcgaacagctctggcaacaattagacgaccagcaaaccgcttttgaactgacaattgaaagtgaattaccagctgaacggggaatgggctcttctgcggccaccgccattgcgattattcgcgccctctttactttttatcatcaagaattaaaccaacgcgacttacttaaactagcagctctttcagaaaatatgattcatggtaatccaagtggcattgatgcggccaccacgggtgcaacgacccccctttggtttgttaaaggccaaccgctacaaccattaccgattgaccttcaaggctacttggtaatcgctgacagcggacaaaaggggcaaaccggtcccgccgttaaaagcgttcaaaatcaaatgatgtcaaatcctgaacgtactcagccgctcattacccacattggggaattgagtcaagctgtcgcaaccaatatcgctcaaaatcagttagtggcactgggccagaatcttagcttagcgcaagatgatttacgagtcttaggggtcagcaattcacgtatcgatgctttaatcgaaactgccaatcaagccggtagcttggggactaaattgactggtggcggacgtggtggttgcattattgcactggctaaaacacaaacagatgccaaacggcttgcacaacaattacgccaagccggcgcgattaagacttggattcaaccattacacgcactacacactaattaactgcagagtctagttaaagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgcttaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 108 - DNA, 6827 bp, Artificial Sequence
  Synthetic Construct
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatgtgtgcgacctcttctcaatttactcagattaccgagcataattcccgtcgttccgcaaactatcagccaaacctgtggaatttcgaattcctgcaatccctggagaacgacctgaaagtggaaaagctggaggagaaagcgaccaaactggaggaagaagttcgctgcatgatcaaccgtgtagacacccagccgctgtccctgctggagctgatcgacgatgtgcagcgcctgggtctgacctacaaatttgaaaaagacatcattaaagccctggaaaacatcgtactgctggacgaaaacaaaaagaacaaatctgacctgcacgcaaccgctctgtctttccgtctgctgcgtcagcacggtttcgaggtttctcaggatgtttttgagcgtttcaaggataaagaaggtggtttcagcggtgaactgaaaggtgacgtccaaggcctgctgagcctgtatgaagcgtcttacctgggtttcgagggtgagaacctgctggaggaggcgcgtaccttttccatcacccacctgaagaacaacctgaaagaaggcattaataccaaggttgcagaacaagtgagccacgccctggaactgccatatcaccagcgtctgcaccgtctggaggcacgttggttcctggataaatacgaaccgaaagaaccgcatcaccagctgctgctggagctggcgaagctggattttaacatggtacagaccctgcaccagaaagagctgcaagatctgtcccgctggtggaccgagatgggcctggctagcaaactggattttgtacgcgaccgcctgatggaagtttatttctgggcactgggtatggcgccagacccgcagtttggtgaatgtcgcaaagctgttactaaaatgtttggtctggtgacgatcatcgatgacgtgtatgacgtttatggcactctggacgaactgcaactgttcaccgatgctgtagagcgctgggacgttaacgctattaacaccctgccggactatatgaaactgtgtttcctggcactgtacaacaccgttaacgacacgtcctattctattctgaaagagaaaggtcataacaacctgtcctatctgacgaaaagctggcgtgaactgtgcaaagcctttctgcaagaggcgaaatggtccaacaacaaaattatcccggctttctccaagtacctggaaaacgccagcgtttcctcctccggtgtagcgctgctggcgccgtcttacttttccgtatgccagcagcaggaagacatctccgaccacgcgctgcgttccctgaccgacttccatggtctggtgcgttctagctgcgttatcttccgcctgtgcaacgatctggccacctctgcggcggagctggaacgtggcgagactaccaattctatcattagctacatgcacgaaaacgatggtaccagcgaggaacaggcccgcgaagaactgcgtaaactgatcgacgccgaatggaaaaagatgaatcgtgaacgcgttagcgactccaccctgctgcctaaagcgttcatggaaatcgcagttaacatggcacgtgtttcccactgcacctaccagtatggcgatggtctgggtcgcccagactacgcgactgaaaaccgcatcaaactgctgctgattgaccctttcccgattaaccagctgatgtatgtctaactgcataaaggaggtaaaaaaacatgacaaaaaaagttggtgtcggtcaggcacatagtaagataattttaataggggaacatgcggtcgtttacggttatcctgccatttccctgcctcttttggaagtggaggtgacctgtaaggtagttcctgcagagagtccttggcgcctttatgaggaggataccttgtccatggcggtttatgcctcactggagtatttgaatatcacagaagcctgcattcgttgtgagattgactcggctatccctgagaaacgggggatgggttcgtcagcggctatcagcatagcggccattcgtgcggtatttgactactatcaggctgatctgcctcatgatgtactagaaatcttggtcaatcgagctgaaatgattgcccatatgaatcctagtggtttggatgctaagacctgtctcagtgaccaacctattcgctttatcaagaacgtaggatttacagaacttgagatggatttatccgcctatttggtgattgccgatacgggtgtttatggtcatactcgtgaagccatccaagtggttcaaaataagggcaaggatgccctaccgtttttgcatgccttgggagaattaacccagcaggcagaaattgcgatttcacaaaaagatgctgaagggctgggacaaatcctcagtcaagcacatttacatttaaaagaaattggtgtcagtagccttgaggcagactctttggttgaaacagctcttagtcatggtgctctgggtgccaagatgagcggtggtgggctaggaggttgtatcatagccttggtaaccaatttgacacacgcacaagaactagcagaaagattagaagagaaaggagctgttcagacatggatagagagcctgtgactgcagagtctagttaaagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgcttaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 109 - DNA, 6980 bp, Artificial Sequence
  Synthetic Construct
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatgtgtgcgacctcttctcaatttactcagattaccgagcataattcccgtcgttccgcaaactatcagccaaacctgtggaatttcgaattcctgcaatccctggagaacgacctgaaagtggaaaagctggaggagaaagcgaccaaactggaggaagaagttcgctgcatgatcaaccgtgtagacacccagccgctgtccctgctggagctgatcgacgatgtgcagcgcctgggtctgacctacaaatttgaaaaagacatcattaaagccctggaaaacatcgtactgctggacgaaaacaaaaagaacaaatctgacctgcacgcaaccgctctgtctttccgtctgctgcgtcagcacggtttcgaggtttctcaggatgtttttgagcgtttcaaggataaagaaggtggtttcagcggtgaactgaaaggtgacgtccaaggcctgctgagcctgtatgaagcgtcttacctgggtttcgagggtgagaacctgctggaggaggcgcgtaccttttccatcacccacctgaagaacaacctgaaagaaggcattaataccaaggttgcagaacaagtgagccacgccctggaactgccatatcaccagcgtctgcaccgtctggaggcacgttggttcctggataaatacgaaccgaaagaaccgcatcaccagctgctgctggagctggcgaagctggattttaacatggtacagaccctgcaccagaaagagctgcaagatctgtcccgctggtggaccgagatgggcctggctagcaaactggattttgtacgcgaccgcctgatggaagtttatttctgggcactgggtatggcgccagacccgcagtttggtgaatgtcgcaaagctgttactaaaatgtttggtctggtgacgatcatcgatgacgtgtatgacgtttatggcactctggacgaactgcaactgttcaccgatgctgtagagcgctgggacgttaacgctattaacaccctgccggactatatgaaactgtgtttcctggcactgtacaacaccgttaacgacacgtcctattctattctgaaagagaaaggtcataacaacctgtcctatctgacgaaaagctggcgtgaactgtgcaaagcctttctgcaagaggcgaaatggtccaacaacaaaattatcccggctttctccaagtacctggaaaacgccagcgtttcctcctccggtgtagcgctgctggcgccgtcttacttttccgtatgccagcagcaggaagacatctccgaccacgcgctgcgttccctgaccgacttccatggtctggtgcgttctagctgcgttatcttccgcctgtgcaacgatctggccacctctgcggcggagctggaacgtggcgagactaccaattctatcattagctacatgcacgaaaacgatggtaccagcgaggaacaggcccgcgaagaactgcgtaaactgatcgacgccgaatggaaaaagatgaatcgtgaacgcgttagcgactccaccctgctgcctaaagcgttcatggaaatcgcagttaacatggcacgtgtttcccactgcacctaccagtatggcgatggtctgggtcgcccagactacgcgactgaaaaccgcatcaaactgctgctgattgaccctttcccgattaaccagctgatgtatgtctaactgcataaaggaggtaaaaaaacatgcaaaaacgccaacgtgaactttccgcgcttactttgccaacctctgcggaaggtgtatccgaaagccaccgtgcccgttccgtaggcatcggccgtgcacatgctaaagcaatcctgttgggcgaacacgctgtagtatacggcgctccagctctggcactccctatcccgcaactgaccgttacggcgagcgttggctggtccagcgaggcttcggatagcgctggtggtctgtcttacactatgactggcaccccgtctcgtgcgcttgtgacccaggcaagcgacggtctgcaccgtctgactgctgaatttatggcacgtatgggcgtgaccaatgctccgcacctggatgttatcctggatggtgctatcccgcacggtcgtggcctgggtagctctgctgccggttcccgcgctatcgctctggcgctggcggacctgttcggtcacgaactggctgagcacaccgcgtatgaactcgtccagacggctgagaacatggcacacggccgtgcttctggcgtagacgcaatgacggtaggtgcaagccgtccacttctgttccaacagggccgtaccgaacgtctggcgatcggttgtgactccctgttcatcgtcgcggattccggtgtgccgggtagcaccaaggaagcggtcgaaatgctgcgtgaaggtttcacgcgcagcgcaggcacccaggagcgtttcgtaggccgtgccaccgagctgaccgaggcagcgcgtcaagcactggctgatggccgtccggaggagctgggctcccagctgacttactaccacgaactgcttcacgaagcccgcctgagcaccgacggcatcgacgccctggtggaagcggctttgaaggccggcagcctgggcgcgaaaatcaccggcggtggcctgggcggttgtatgattgctcaggcccgtcctgaacaggcccgtgaagttacccgccaactgcacgaggccggtgcagtacagacctgggttgttccgctgaaaggccttgataaccatgcgcagtaaagtctagttaaagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgcttaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 110 - DNA, 45 bp, Artificial Sequence
  Synthetic Construct
accgttcgttcttatcgaaactaaaaaaaaccggccttggccccg

SEQ ID NO: 111 - DNA, 7063 bp, Artificial Sequence
  Synthetic Construct
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggccatcatcatcatcatcatcatcatcatcacagcagcggccatatcgaaggtcgtcatatgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcataagctaatttgcgataggccatatgctcgaggatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa

SEQ ID NO: 112 - DNA, 6692 bp, Artificial Sequence
  Synthetic Construct
aagggcgagctcaacgatccggctgctaacaaagcccgaaaggaagctgagttggctgctgccaccgctgagcaataactagcataaccccttggggcctctaaacgggtcttgaggagttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgattgaacaagatggattgcacgcaggttctccggccgcttgggtggagaggctattcggctatgactgggcacaactgacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgggcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgcggggttctcatcatcatcatcatcatggtatggctagcatgactggtggacagcaaatgggtcgggatctgtacgacgatgacgataaggatcatcccttcaccatgcaaacgagtgtgggaaacagtcacgctaagattattttgattggagagcatgctgtcgtgtacgggcaacccgcgattgcgcttcctatttttgcagtccagacaacggttcatgtcacggcacaaccaacactgagcgaacccttgattaacagcccttattacagtggccctttaagtcaagcgattggtcagatgcaaggtattcgtcgattaatcgaacagctctggcaacaattagacgaccagcaaaccgcttttgaactgacaattgaaagtgaattaccagctgaacggggaatgggctcttctgcggccaccgccattgcgattattcgcgccctctttactttttatcatcaagaattaaaccaacgcgacttacttaaactagcagctctttcagaaaatatgattcatggtaatccaagtggcattgatgcggccaccacgggtgcaacgacccccctttggtttgttaaaggccaaccgctacaaccattaccgattgaccttcaaggctacttggtaatcgctgacagcggacaaaaggggcaaaccggtcccgccgttaaaagcgttcaaaatcaaatgatgtcaaatcctgaacgtactcagccgctcattacccacattggggaattgagtcaagctgtcgcaaccaatatcgctcaaaatcagttagtggcactgggccagaatcttagcttagcgcaagatgatttacgagtcttaggggtcagcaattcacgtatcgatgctttaatcgaaactgccaatcaagccggtagcttggggactaaattgactggtggcggacgtggtggttgcattattgcactggctaaaacacaaacagatgccaaacggcttgcacaacaattacgccaagccggcgcgattaagacttggattcaaccattacacgcactacacactaattaaaca

SEQ ID NO: 113 - DNA, 6623 bp, Artificial Sequence
  Synthetic Construct
aagggcgagctcaacgatccggctgctaacaaagcccgaaaggaagctgagttggctgctgccaccgctgagcaataactagcataaccccttggggcctctaaacgggtcttgaggagttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgattgaacaagatggattgcacgcaggttctccggccgcttgggtggagaggctattcggctatgactgggcacaactgacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgggcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgcggggttctcatcatcatcatcatcatggtatggctagcatgactggtggacagcaaatgggtcgggatctgtacgacgatgacgataaggatcatcccttcaccatgacaaaaaaagttggtgtcggtcaggcacatagtaagataattttaataggggaacatgcggtcgtttacggttatcctgccatttccctgcctcttttggaagtggaggtgacctgtaaggtagttcctgcagagagtccttggcgcctttatgaggaggataccttgtccatggcggtttatgcctcactggagtatttgaatatcacagaagcctgcattcgttgtgagattgactcggctatccctgagaaacgggggatgggttcgtcagcggctatcagcatagcggccattcgtgcggtatttgactactatcaggctgatctgcctcatgatgtactagaaatcttggtcaatcgagctgaaatgattgcccatatgaatcctagtggtttggatgctaagacctgtctcagtgaccaacctattcgctttatcaagaacgtaggatttacagaacttgagatggatttatccgcctatttggtgattgccgatacgggtgtttatggtcatactcgtgaagccatccaagtggttcaaaataagggcaaggatgccctaccgtttttgcatgccttgggagaattaacccagcaggcagaaattgcgatttcacaaaaagatgctgaagggctgggacaaatcctcagtcaagcacatttacatttaaaagaaattggtgtcagtagccttgaggcagactctttggttgaaacagctcttagtcatggtgctctgggtgccaagatgagcggtggtgggctaggaggttgtatcatagccttggtaaccaatttgacacacgcacaagaactagcagaaagattagaagagaaaggagctgttcagacatggatagagagcctgtgacag

SEQ ID NO: 114 - DNA, 4334 bp, Artificial Sequence
  Synthetic Construct
cattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggatccgagctcgagatctgcagctggtaccatatgggaattcgaagctttctagaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgcttaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcc

SEQ ID NO: 115 - DNA, 31 bp, Artificial Sequence; identical to SEQ ID NO: 88
  Synthetic Construct

SEQ ID NO: 116 - DNA, 23 bp, Artificial Sequence
  Synthetic Construct
ccaggcaaattctgttttatcag

SEQ ID NO: 117 - DNA, 35 bp, Artificial Sequence
  Synthetic Construct
cagcagcagcatatgtcattaccgttcttaacttc

SEQ ID NO: 118 - DNA, 37 bp, Artificial Sequence
  Synthetic Construct
cagcagcagcatatggcctatcgcaaattagcttatg

SEQ ID NO: 119 - DNA, 24 bp, Artificial Sequence
  Synthetic Construct
caccatgcaaaaacgccaacgtga

SEQ ID NO: 120 - DNA, 23 bp, Artificial Sequence
  Synthetic Construct
ttactgcgcatggttatcaaggc

SEQ ID NO: 121 - DNA, 22 bp, Artificial Sequence
  Synthetic Construct
caccatggtatcctgttctgcg

SEQ ID NO: 122 - DNA, 22 bp, Artificial Sequence
  Synthetic Construct
ttaatctactttcagaccttgc

SEQ ID NO: 123 - DNA, 50 bp, Artificial Sequence
  Synthetic Construct
gcgaacgatgcataaaggaggtaaaaaaacatgcaaaaacgccaacgtga

SEQ ID NO: 124 - DNA, 66 bp, Artificial Sequence
  Synthetic Construct
gcgaacgatgcataaaggaggtaaaaaaacatggtatcctgttctgcgccgggtaagatttacctg

SEQ ID NO: 125 - DNA, 54 bp, Artificial Sequence
  Synthetic Construct
gcgaacgatgcataaaggaggtaaaaaaacatgacaaaaaaagttggtgtcggt

SEQ ID NO: 126 - DNA, 58 bp, Artificial Sequence
  Synthetic Construct
gggcccgtttaaactttaactagactctgcagtcacaggctctctatccatgtctgaa

SEQ ID NO: 127 - DNA, 52 bp, Artificial Sequence
  Synthetic Construct
gcgaacgatgcataaaggaggtaaaaaaacatgcaaacgagtgtgggaaaca

SEQ ID NO: 128 - DNA, 62 bp, Artificial Sequence
  Synthetic Construct
gggcccgtttaaactttaactagactctgcagttaattagtgtgtagtgcgtgtaatggttg

SEQ ID NO: 129 - DNA, 57 bp, Artificial Sequence
  Synthetic Construct
gcgaacgatgcataaaggaggtaaaaaaacatgtcattaccgttcttaacttctgca

SEQ ID NO: 130 - DNA, 58 bp, Artificial Sequence
  Synthetic Construct
gggcccgtttaaactttaactagactctgcagttatgaagtccatggtaaattcgtgt

SEQ ID NO: 131 - DNA, 49 bp, Artificial Sequence
  Synthetic Construct
gggcccgtttaaactttaactagactttactgcgcatggttatcaaggc

SEQ ID NO: 132 - DNA, 48 bp, Artificial Sequence
  Synthetic Construct
gggcccgtttaaactttaactagactttaatctactttcagaccttgc

SEQ ID NO: 133 - DNA, 34 bp, Artificial Sequence
  Synthetic Construct
caccatgcaaacgagtgtgggaaacagtcacgct

SEQ ID NO: 134 - DNA, 30 bp, Artificial Sequence
  Synthetic Construct
tgtttaattagtgtgtagtgcgtgtaatgg

SEQ ID NO: 135 - DNA, 35 bp, Artificial Sequence
  Synthetic Construct
caccatgacaaaaaaagttggtgtcggtcaggcac

SEQ ID NO: 136 - DNA, 30 bp, Artificial Sequence
  Synthetic Construct
ctgtcacaggctctctatccatgtctgaac

SEQ ID NO: 137 - DNA, 20 bp, Artificial Sequence
  Synthetic Construct
taatacgactcactataggg

SEQ ID NO: 138 - DNA, 19 bp, Artificial Sequence
  Synthetic Construct
gctagttattgctcagcgg

SEQ ID NO: 139 - DNA, 48 bp, Artificial Sequence
  Synthetic Construct
tcatccggctcgtataatgtgtggtcacacaggaaacagcgccgctga

SEQ ID NO: 140 - DNA, 48 bp, Artificial Sequence
  Synthetic Construct
tcagcggcgctgtttcctgtgtgaccacacattatacgagccggatga

SEQ ID NO: 141 - DNA, 8684 bp, Artificial Sequence
  Synthetic Construct
ttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggatccgagctcaggaggtaaaaaaacatgaaaacagtagttattattgatgcattacgaacaccaattggaaaatataaaggcagcttaagtcaagtaagtgccgtagacttaggaacacatgttacaacacaacttttaaaaagacattccactatttctgaagaaattgatcaagtaatctttggaaatgttttacaagctggaaatggccaaaatcccgcacgacaaatagcaataaacagcggtttgtctcatgaaattcccgcaatgacggttaatgaggtctgcggatcaggaatgaaggccgttattttggcgaaacaattgattcaattaggagaagcggaagttttaattgctggcgggattgagaatatgtcccaagcacctaaattacaacgttttaattacgaaacagaaagctacgatgcgcctttttctagtatgatgtatgatggattaacggatgcctttagtggtcaggcaatgggcttaactgctgaaaatgtggccgaaaagtatcatgtaactagagaagagcaagatcaattttctgtacattcacaattaaaagcagctcaagcacaagcagaagggatattcgctgacgaaatagccccattagaagtatcaggaacgcttgtggagaaagatgaagggattcgccctaattcgagcgttgagaagctaggaacgcttaaaacagtttttaaagaagacggtactgtaacagcagggaatgcatcaaccattaatgatggggcttctgctttgattattgcttcacaagaatatgccgaagcacacggtcttccttatttagctattattcgagacagtgtggaagtcggtattgatccagcctatatgggaatttcgccgattaaagccattcaaaaactgttagcgcgcaatcaacttactacggaagaaattgatctgtatgaaatcaacgaagcatttgcagcaacttcaatcgtggtccaaagagaactggctttaccagaggaaaaggtcaacatttatggtggcggtatttcattaggtcatgcgattggtgccacaggtgctcgtttattaacgagtttaagttatcaattaaatcaaaaagaaaagaaatatggagtggcttctttatgtatcggcggtggcttaggactcgctatgctactagagagacctcagcaaaaaaaaaacagccgattttatcaaatgagtcctgaggaacgcctggcttctcttcttaatgaaggccagatttctgctgatacaaaaaaagaatttgaaaatacggctttatcttcgcagattgccaatcatatgattgaaaatcaaatcagtgaaacagaagtgccgatgggcgttggcttacatttaacagtggacgaaactgattatttggtaccaatggcgacagaagagccctcagttattgcggctttgagtaatggtgcaaaaatagcacaaggatttaaaacagtgaatcaacaacgcttaatgcgtggacaaatcgttttttacgatgttgcagatcccgagtcattgattgataaactacaagtaagagaagcggaagtttttcaacaagcagagttaagttatccatctatcgttaaacggggcggcggcttaagagatttgcaatatcgtacttttgatgaatcatttgtatctgtcgactttttagtagatgttaaggatgcaatgggggcaaatatcgttaacgctatgttggaaggtgtggccgagttgttccgtgaatggtttgcggagcaaaagattttattcagtattttaagtaattatgccacggagtcggttgttacgatgaaaacggctattccagtttcacgtttaagtaaggggagcaatggccgggaaattgctgaaaaaattgttttagcttcacgctatgcttcattagatccttatcgggcagtcacgcataacaaaggaatcatgaatggcattgaagctgtagttttagctacaggaaatgatacacgcgctgttagcgcttcttgtcatgcttttgcggtgaaggaaggtcgctaccaaggcttgactagttggacgctggatggcgaacaactaattggtgaaatttcagttccgcttgctttagccacggttggcggtgccacaaaagtcttacctaaatctcaagcagctgctgatttgttagcagtgacggatgcaaaagaactaagtcgagtagtagcggctgttggtttggcacaaaatttagcggcgttacgggccttagtctctgaaggaattcaaaaaggacacatggctctacaagcacgttctttagcgatgacggtcggagctactggtaaagaagttgaggcagtcgctcaacaattaaaacgtcaaaaaacgatgaaccaagaccgagccatggctattttaaatgatttaagaaaacaataaaggaggtaaaaaaacatgacaattgggattgataaaattagtttttttgtgcccccttattatattgatatgacggcactggctgaagccagaaatgtagaccctggaaaatttcatattggtattgggcaagaccaaatggcggtgaacccaatcagccaagatattgtgacatttgcagccaatgccgcagaagcgatcttgaccaaagaagataaagaggccattgatatggtgattgtcgggactgagtccagtatcgatgagtcaaaagcggccgcagttgtcttacatcgtttaatggggattcaacctttcgctcgctctttcgaaatcaaggaagcttgttacggagcaacagcaggcttacagttagctaagaatcacgtagccttacatccagataaaaaagtcttggtcgtagcggcagatattgcaaaatatggcttaaattctggcggtgagcctacacaaggagctggggcggttgcaatgttagttgctagtgaaccgcgcattttggctttaaaagaggataatgtgatgctgacgcaagatatctatgacttttggcgtccaacaggccacccgtatcctatggtcgatggtcctttgtcaaacgaaacctacatccaatcttttgcccaagtctgggatgaacataaaaaacgaaccggtcttgattttgcagattatgatgctttagcgttccatattccttacacaaaaatgggcaaaaaagccttattagcaaaaatctccgaccaaactgaagcagaacaggaacgaattttagcccgttatgaagaaagtatcgtctatagtcgtcgcgtaggaaacttgtatacgggttcactttatctgggactcatttcccttttagaaaatgcaacgactttaaccgcaggcaatcaaattggtttattcagttatggttctggtgctgtcgctgaatttttcactggtgaattagtagctggttatcaaaatcatttacaaaaagaaactcatttagcactgctggataatcggacagaactttctatcgctgaatatgaagccatgtttgcagaaactttagacacagacattgatcaaacgttagaagatgaattaaaatatagtatttctgctattaataataccgttcgttcttatcgaaactaagagatctgcagctggtaccatatgggaattcgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcgggaattcgcgttggccgattcattaatgcagattctgaaatgagctgttgacaattaatcatccggctcgtataatgtgtgga

SEQ ID NO: 142 - DNA, 21 bp, Artificial Sequence
  Synthetic Construct
cttctcagggcgttttatggc

SEQ ID NO: 143 - DNA, 20 bp, Artificial Sequence
  Synthetic Construct
gttgagctaacaacggatcc

SEQ ID NO: 144 - DNA, 23 bp, Artificial Sequence
  Synthetic Construct
gactgtcaacccaaacgtcaatg

SEQ ID NO: 145 - DNA, 10022 bp, Artificial Sequence
  Synthetic Construct
cccgtcttactgtcgggaattcgcgttggccgattcattaatgcagattctgaaatgagctgttgacaattaatcatccggctcgtataatgtgtggtcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggatccgagctcggatccactagtaacggccgccagtgtgctggaattcgcccttaggaggtaaaaaaacatgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcataagctaatttgcgataggcctgcacccttaaggaggaaaaaaacatgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaataacttaaggtagctgcatgcagaattcgcccttaaggaggaaaaaaaaatgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataagatcaattcgctgcatcgcccttaggaggtaaaaaaaaatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataacaacgcgtcctgcagctggtaccatatgggaattcgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggctta

SEQ ID NO: 146 - DNA, 72 bp, Artificial Sequence
  Synthetic Construct
ggctggcggcgttttgctttttattctgtctcaactctggatgtttcatgaattaaccctcactaaagggcg

SEQ ID NO: 147 - DNA, 73 bp, Artificial Sequence
  Synthetic Construct
aagccctacgctaacaaatagcgcgactctctgtagccggattatcctcataatacgactcactatagggctc

SEQ ID NO: 148 - DNA, 26 bp, Artificial Sequence
  Synthetic Construct
acgccgctcagtagatccttgcggat

SEQ ID NO: 149 - DNA, 27 bp, Artificial Sequence
  Synthetic Construct
ctacttacgatcagatggcgcagacta

SEQ ID NO: 150 - DNA, 22 bp, Artificial Sequence
  Synthetic Construct
cgagactagtgagacgtgctac

SEQ ID NO: 151 - DNA, 24 bp, Artificial Sequence
  Synthetic Construct
aaagaccgaccaagcgacgtctga

(19) United States
(12) Patent Application Publication
(10) Pub. No.: US 2010/0184178 A1
(43) Pub. Date: Jul. 22, 2010

(54) INCREASED ISOPRENE PRODUCTION USING MEVALONATE KINASE AND ISOPRENE SYNTHASE

(76) Inventors: Zachary Quinn Beck, Palo Alto, CA (US); Anthony Rudolf Calabria, San Mateo, CA (US); Michael Charles Miller, San Francisco, CA (US); Dmitrii V. Vaviline, Palo Alto, CA (US); Derek H. Wells, Palo Alto, CA (US)

Correspondence Address: MORRISON & FOERSTER LLP, 755 PAGE MILL RD, PALO ALTO, CA, 94304-1018, US

(21) Appl. No.: 12/560,305

(22) Filed: Sep. 15, 2009

Related U.S. Application Data
(60) Provisional application No. 61/097,189, filed on Sep. 15, 2008.

(51) Int. Cl. C12P 5/02; C12N 9/12

(52) U.S. Cl. 435/167; 435/194

(57) ABSTRACT

The invention features methods for producing isoprene from cultured cells having increased expression levels and/or activity levels of a mevalonate kinase polypeptide and an isoprene synthase polypeptide. The invention also provides methods for producing isoprene from cultured cells having reduced accumulation of intermediates (such as mevalonate, isopentenyl diphosphate, 3,3-dimethylallyl diphosphate, geranyl diphosphate, or farnesyl diphosphate) in the biosynthesis of isoprene or isoprenoids that may otherwise cause undesirable amounts of growth inhibition, toxicity, or cell death. The resulting isoprene compositions may have increased yields and/or purity of isoprene.

### CROSS-REFERENCE TO RELATED APPLICATIONS

This applications claims the benefit of U.S. Provisional patent application 61/097,189, filed on Sep. 15, 2008, the contents of which are hereby incorporated by reference in its entirety.

### BACKGROUND OF THE INVENTION

Isoprene (2-methyl-1,3-butadiene) is the critical starting material for a variety of synthetic polymers, most notably synthetic rubbers. Isoprene is naturally produced by a variety of microbial, plant, and animal species. In particular, two pathways have been identified for the biosynthesis of isoprene: the mevalonate (MVA) pathway and the non-mevalonate (DXP) pathway (FIGS. 19A and 19B). However, the yield of isoprene from naturally-occurring organisms is commercially unattractive. About 800,000 tons per year of cis-polyisoprene are produced from the polymerization of isoprene; most of this polyisoprene is used in the tire and rubber industry. Isoprene is also copolymerized for use as a synthetic elastomer in other products such as footwear, mechanical products, medical products, sporting goods, and latex.

Currently, the tire and rubber industry is based on the use of natural and synthetic rubber. Natural rubber is obtained from the milky juice of rubber trees or plants found in the rainforests of Africa. Synthetic rubber is based primarily on butadiene polymers. For these polymers, butadiene is obtained as a co-product from ethylene and propylene manufacture.

While isoprene can be obtained by fractionating petroleum, the purification of this material is expensive and time-consuming. Petroleum cracking of the C5 stream of hydrocarbons produces only about 15% isoprene. Thus, more economical methods for producing isoprene are needed. In particular, methods that produce isoprene at rates, titers, and purity that are sufficient to meet the demands of a robust commercial process are desirable. Also desired are systems for producing isoprene from inexpensive starting materials.

### BRIEF SUMMARY OF THE INVENTION

The invention provides compositions, methods and systems for isoprene, making isoprene and using isoprene. In one aspect, the invention provides for cells in culture comprising a nucleic acid encoding a heterologous isoprene synthase polypeptide and one or more nucleic acids encoding MVA pathway polypeptides, wherein the cells further comprise i) one or more copies of a nucleic acid encoding a mevalonate kinase polypeptide, or ii) a nucleic acid encoding a mevalonate kinase polypeptide under the control of a strong promoter, and wherein the cells express the mevalonate kinase polypeptide at a level that is at least about 2-fold higher than the level of expression in cells that do not comprise one or more copies of a nucleic acid encoding a mevalonate kinase polypeptide or a nucleic acid encoding a mevalonate kinase polypeptide under the control of a strong promoter. In one embodiment, the cells produce greater than about 400 nmole/g_{wcm}/hr of isoprene. In another embodiment, the mevalonate kinase polypeptide is M. mazei mevalonate kinase. In another embodiment, the MVA pathway polypeptide is selected from the group consisting of Lactobacillus mevalonate kinase polypeptide, Lactobacillus sakei mevalonate kinase polypeptide, yeast mevalonate kinase polypeptide, Saccharomyces cerevisiae mevalonate kinase polypeptide, Streptococcus mevalonate kinase polypeptide, Streptococcus pneumoniae mevalonate kinase polypeptide, and Streptomyces mevalonate kinase polypeptide, Streptomyces CL190 mevalonate kinase polypeptide. In another embodiment, the MVA pathway polypeptide is a polypeptide from Saccharomyces cerevicia or Enterococcus faecalis.

In another aspect, the invention features cells in culture that produce isoprene. In some embodiments, the cells in culture comprise a nucleic acid (such as a heterologous nucleic acid or a duplicate copy of an endogenous nucleic acid) encoding an isoprene synthase polypeptide and a nucleic acid (such as a heterologous nucleic acid or a duplicate copy of an endogenous nucleic acid) encoding a mevalonate kinase polypeptide as a first MVA pathway polypeptide. In some embodiments, the cells express the mevalonate kinase polypeptide at a level that is at least about any of 2, 5, 10, 20, 30, 40, 50, 60, 70, 80, 90, 100, 125, 150, 200, 225, 250, 275, 300, 350, 400, 450, or 500-fold higher than the level of expression of a second MVA pathway polypeptide in the cell. In some embodiments, the nucleic acid encoding a mevalonate kinase polypeptide is under the control of a strong promoter. In some embodiments, the nucleic acid encoding a mevalonate kinase polypeptide is under the control of a strong promoter, and the second MVA pathway polypeptide is not under the control of a strong promoter. In various embodiments, the second MVA pathway polypeptide is an acetyl-CoA acetyltransferase polypeptide, 3-hydroxy-3-methylglutaryl-CoA synthase polypeptide, 3-hydroxy-3-methylglutaryl-CoA reductase polypeptide, phosphomevalonate kinase polypeptide, diphosphomevalonate decarboxylase polypeptide, or isopentenyl-diphosphate delta-isomerase polypeptide. In some embodiments, the cells express an entire MVA pathway. In some embodiments, the mevalonate kinase polypeptide is an archaeal mevalonate kinase polypeptide (e.g., a Methanosarcina mazei mevalonate kinase polypeptide), a Lactobacillus mevalonate kinase polypeptide (e.g., a Lactobacillus sakei mevalonate kinase polypeptide), a yeast mevalonate kinase polypeptide (e.g., a Saccharomyces cerevisia mevalonate kinase polypeptide), a Streptococcus mevalonate kinase polypeptide (e.g., a Streptococcus pneumoniae mevalonate kinase polypeptide), or a Streptomyces mevalonate kinase polypeptide (e.g., a Streptomyces CL190 mevalonate kinase polypeptide). In some embodiments, the cells in culture produce greater than about 400 nmole/g_{wcm}/hr of isoprene. In some embodiments, the cells in culture convert more than about 0.002% of the carbon in a cell culture medium into isoprene.

In some embodiments, the cells in culture comprise a nucleic acid (such as a heterologous nucleic acid or a duplicate copy of an endogenous nucleic acid) encoding an isoprene synthase polypeptide and a nucleic acid (such as a heterologous nucleic acid or a duplicate copy of an endogenous nucleic acid) encoding a mevalonate kinase polypeptide. In some embodiments, (i) the intracellular concentration of 3,3-dimethylallyl diphosphate (DMAPP) is between about 0 to about 25 μmol/g_{dcw}, (ii) the intracellular concentration of isopentenyl diphosphate (IPP) is between about 0 to about 60 μmol/g_{dcw}, (iii) the intracellular concentration of geranyl diphosphate (GPP) is between about 0 to about 8 μmol/g_{dcw}, (iv) the intracellular concentration of farnesyl diphosphate (FPP) is between about 0 to about 6 μmol/g_{dcw}, or (v) any combination of two or more of the foregoing. In some embodiments, the cells express an entire MVA pathway. In some embodiments, the mevalonate kinase polypeptide is an archaeal mevalonate kinase polypeptide (e.g., a Methanosarcina mazei mevalonate kinase polypeptide), a Lactobacillus mevalonate kinase polypeptide (e.g., a Lactobacillus sakei mevalonate kinase polypeptide), a yeast mevalonate kinase polypeptide (e.g., a Saccharomyces cerevisiae mevalonate kinase polypeptide), a Streptococcus mevalonate kinase polypeptide (e.g., a Streptococcus pneumoniae mevalonate kinase polypeptide), or a Streptomyces mevalonate kinase polypeptide (e.g., a Streptomyces CL190 mevalonate kinase polypeptide). In some embodiments, the cells in culture produce greater than about 400 nmole/g_{wcm}/hr of isoprene. In some embodiments, the cells in culture convert more than about 0.002% of the carbon in a cell culture medium into isoprene.

In some embodiments of any of the cells, the cells are cultured in a culture medium that includes a carbon source, such as, but not limited to, a carbohydrate, glycerol, glycerine, dihydroxyacetone, one-carbon source, oil, animal fat, animal oil, fatty acid, lipid, phospholipid, glycerolipid, monoglyceride, diglyceride, triglyceride, renewable carbon source, polypeptide (e.g., a microbial or plant protein or peptide), yeast extract, component from a yeast extract, or any combination of two or more of the foregoing. In some embodiments, the cells are cultured under limited glucose conditions.

In another aspect, the invention features compositions comprising any one or more of the cells described herein. In one aspect, the invention features compositions comprising cells in culture comprising a nucleic acid encoding a heterologous isoprene synthase polypeptide and one or more nucleic acids encoding MVA pathway polypeptides, wherein the cells further comprise i) one or more copies of a nucleic acid encoding a mevalonate kinase polypeptide, or ii) a nucleic acid encoding a mevalonate kinase polypeptide under the control of a strong promoter, and wherein the cells express the mevalonate kinase polypeptide at a level that is at least about 2-fold higher than the level of expression in cells that do not comprise one or more copies of a nucleic acid encoding a mevalonate kinase polypeptide or a nucleic acid encoding a mevalonate kinase polypeptide under the control of a strong promoter.

In one aspect, the invention features methods of producing isoprene, such as methods of using any of the cells described herein to produce isoprene. In one aspect, the invention features methods of producing isoprene, the method comprising (a) culturing cells in culture comprising a nucleic acid encoding a heterologous isoprene synthase polypeptide and one or more nucleic acids encoding MVA pathway polypeptides, wherein the cells further comprise i) one or more copies of a nucleic acid encoding a mevalonate kinase polypeptide, or ii) a nucleic acid encoding a mevalonate kinase polypeptide under the control of a strong promoter, and wherein the cells express the mevalonate kinase polypeptide at a level that is at least about 2-fold higher than the level of expression in cells that do not comprise one or more copies of a nucleic acid encoding a mevalonate kinase polypeptide or a nucleic acid encoding a mevalonate kinase polypeptide under the control of a strong promoter under suitable culture conditions for the production of isoprene, and (b) producing isoprene. In some embodiments, the method involves culturing cells comprising a nucleic acid (such as a heterologous nucleic acid or a duplicate copy of an endogenous nucleic acid) encoding an isoprene synthase polypeptide and a nucleic acid (such as a heterologous nucleic acid or a duplicate copy of an endogenous nucleic acid) encoding a mevalonate kinase polypeptide as a first MVA pathway polypeptide. In some embodiments, the nucleic acid encoding a mevalonate kinase polypeptide is under the control of a strong promoter. In some embodiments, the nucleic acid encoding a mevalonate kinase polypeptide is under the control of a strong promoter, and the second MVA pathway polypeptide is not under the control of a strong promoter. In some embodiments, the cells are cultured under suitable culture conditions for the production of isoprene, and isoprene is produced. In some embodiments, the cells express the mevalonate kinase polypeptide at a level that is at least about any of 2, 5, 10, 20, 30, 40, 50, 60, 70, 80, 90, 100, 125, 150, 200, 225, 250, 275, 300, 350, 400, 450, or 500-fold higher than the level of expression of a second MVA pathway polypeptide in the cell. In various embodiments, the second MVA pathway polypeptide is an acetyl-CoA acetyltransferase polypeptide, 3-hydroxy-3-methylglutaryl-CoA synthase polypeptide, 3-hydroxy-3-methylglutaryl-CoA reductase polypeptide, phosphomevalonate kinase polypeptide, diphosphomevalonate decarboxylase polypeptide, or isopentenyl-diphosphate delta-isomerase polypeptide. In some embodiments, the cells express an entire MVA pathway. In some embodiments, the mevalonate kinase polypeptide is an archaeal mevalonate kinase polypeptide (e.g., a Methanosarcina mazei mevalonate kinase polypeptide), a Lactobacillus mevalonate kinase polypeptide (e.g., a Lactobacillus sakei mevalonate kinase polypeptide), a yeast mevalonate kinase polypeptide (e.g., a Saccharomyces cerevisia mevalonate kinase polypeptide), a Streptococcus mevalonate kinase polypeptide (e.g., a Streptococcus pneumoniae mevalonate kinase polypeptide), or a Streptomyces mevalonate kinase polypeptide (e.g., a Streptomyces CL190 mevalonate kinase polypeptide). In some embodiments, the method involves culturing cells under conditions sufficient to produce greater than about 400 nmole/g_{wcm}/hr of isoprene. In some embodiments, the method includes culturing cells under conditions sufficient to convert more than about 0.002% of the carbon (mol/mol) in a cell culture medium into isoprene.

In some embodiments, the method involves culturing cells comprising a nucleic acid (such as a heterologous nucleic acid or a duplicate copy of an endogenous nucleic acid) encoding an isoprene synthase polypeptide and a nucleic acid (such as a heterologous nucleic acid or a duplicate copy of an endogenous nucleic acid) encoding a mevalonate kinase polypeptide. In some embodiments, (i) the intracellular concentration of DMAPP is between about 0 to about 25 μmol/g_{dcw}, (ii) the intracellular concentration of IPP is between about 0 to about 60 mol/g_{dcw}, (iii) the intracellular concentration of GPP is between about 0 to about 8 μmol/g_{dcw}, (iv) the intracellular concentration of FPP is between about 0 to about 6 μmol/g_{dcw}, or (v) any combination of two or more of the foregoing. In some embodiments, the cells are cultured under suitable culture conditions for the production of isoprene, and isoprene is produced. In some embodiments, the cells express an entire MVA pathway. In some embodiments, the mevalonate kinase polypeptide is an archaeal mevalonate kinase polypeptide (e.g., a Methanosarcina mazei mevalonate kinase polypeptide), a Lactobacillus mevalonate kinase polypeptide (e.g., a Lactobacillus sakei mevalonate kinase polypeptide), a yeast mevalonate kinase polypeptide (e.g., a Saccharomyces cerevisia mevalonate kinase polypeptide), a Streptococcus mevalonate kinase polypeptide (e.g., a Streptococcus pneumoniae mevalonate kinase polypeptide), or a Streptomyces mevalonate kinase polypeptide (e.g., a Streptomyces CL190 mevalonate kinase polypeptide). In some embodiments, the method involves culturing cells under conditions sufficient to produce greater than about 400 nmole/g_{wcm}/hr of isoprene. In some embodiments, the method includes culturing cells under conditions sufficient to convert more than about 0.002% of the carbon (mol/mol) in a cell culture medium into isoprene.

In some embodiments of any of the methods, the method also includes recovering isoprene produced by the cells. In some embodiments, the method includes purifying isoprene produced by the cells. In some embodiments, the method includes polymerizing the isoprene. In some embodiments, the cells are cultured in a culture medium that includes a carbon source, such as, but not limited to, a carbohydrate, glycerol, glycerine, dihydroxyacetone, one-carbon source, oil, animal fat, animal oil, fatty acid, lipid, phospholipid, glycerolipid, monoglyceride, diglyceride, triglyceride, renewable carbon source, polypeptide (e.g., a microbial or plant protein or peptide), yeast extract, component from a yeast extract, or any combination of two or more of the foregoing. In some embodiments, the cells are cultured under limited glucose conditions. In various embodiments, the amount of isoprene produced (such as the total amount of isoprene produced or the amount of isoprene produced per liter of broth per hour per OD₆₀₀) during stationary phase is greater than or about 2 or more times the amount of isoprene produced during the growth phase for the same length of time. In some embodiments, the gas phase comprises greater than or about 9.5% (volume) oxygen, and the concentration of isoprene in the gas phase is less than the lower flammability limit or greater than the upper flammability limit. In particular embodiments, (i) the concentration of isoprene in the gas phase is less than the lower flammability limit or greater than the upper flammability limit, and (ii) the cells produce greater than about 400 nmole/g_{wcm}/hr of isoprene.

In some embodiments of any of the compositions, systems, and methods of the invention, a mevalonate kinase polypeptide and/or an isoprene synthase polypeptide is expressed a level that is at least about any of 2, 5, 10, 20, 30, 40, 50, 60, 70, 80, 90, 100, 125, 150, 200, 225, 250, 275, 300, 350, 400, 450, or 500-fold (i) higher than the level of expression of a second MVA pathway polypeptide (such as an acetyl-CoA acetyltransferase polypeptide, 3-hydroxy-3-methylglutaryl-CoA synthase polypeptide, 3-hydroxy-3-methylglutaryl-CoA reductase polypeptide, phosphomevalonate kinase polypeptide, diphosphomevalonate decarboxylase polypeptide, or isopentenyl-diphosphate delta-isomerase polypeptide) or (ii) higher than the level of expression of all other MVA pathway polypeptides in the cell. In particular embodiments, the mevalonate kinase polypeptide and/or an isoprene synthase polypeptide is expressed a level that is at least about any of 2, 5, 10, 12, 14, 16, 18, 20, 30, 40, 50, 60, 70, 80, 90, 100, 125, 150, 200, 225, 250, 275, 300, 350, 400, 450, or 500-fold higher than the level of expression of an acetyl-CoA acetyltransferase polypeptide, 3-hydroxy-3-methylglutaryl-CoA synthase polypeptide, and 3-hydroxy-3-methylglutaryl-CoA reductase polypeptide. In particular embodiments, the mevalonate kinase polypeptide and/or an isoprene synthase polypeptide is expressed a level that is at least about any of 2, 5, 10, 20, 30, 40, 50, 60, 70, 80, 90, 100, 125, 150, 200, 225, 250, 275, 300, 350, 400, 450, or 500-fold higher than the level of expression of an phosphomevalonate kinase polypeptide, diphosphomevalonate decarboxylase polypeptide, and isopentenyl-diphosphate delta-isomerase polypeptide. In some embodiments, the total amount of mevalonate kinase polypeptide is similar to the total amount of isoprene synthase polypeptide. For example, in some embodiments, the total amount of mevalonate kinase polypeptide is within about any of 10, 8, 6, 4, 2, 1, or 0.5-fold higher or lower than the total amount of isoprene synthase polypeptide (e.g., the amount of mevalonate kinase polypeptide may be between about 10-fold lower to about 10-fold higher than the amount of isoprene synthase polypeptide).

In some embodiments of any of the compositions, systems, and methods of the invention, a mevalonate kinase RNA molecule and/or an isoprene synthase RNA molecule is expressed a level that is at least about any of 2, 5, 10, 20, 30, 40, 50, 60, 70, 80, 90, 100, 125, 150, 200, 225, 250, 275, 300, 350, 400, 450, or 500-fold (i) higher than the level of expression of a second MVA pathway RNA molecule (such as an acetyl-CoA acetyltransferase RNA molecule, 3-hydroxy-3-methylglutaryl-CoA synthase RNA molecule, 3-hydroxy-3-methylglutaryl-CoA reductase RNA molecule, phosphomevalonate kinase RNA molecule, diphosphomevalonate decarboxylase RNA molecule, or isopentenyl-diphosphate delta-isomerase RNA molecule) or (ii) higher than the level of expression of all other MVA pathway RNA molecules in the cell. In particular embodiments, the mevalonate kinase RNA molecule and/or an isoprene synthase RNA molecule is expressed a level that is at least about any of 2, 5, 10, 12, 14, 16, 18, 20, 30, 40, 50, 60, 70, 80, 90, 100, 125, 150, 200, 225, 250, 275, 300, 350, 400, 450, or 500-fold higher than the level of expression of an acetyl-CoA acetyltransferase RNA molecule, 3-hydroxy-3-methylglutaryl-CoA synthase RNA molecule, and 3-hydroxy-3-methylglutaryl-CoA reductase RNA molecule. In particular embodiments, the mevalonate kinase RNA molecule and/or an isoprene synthase RNA molecule is expressed a level that is at least about any of 2, 5, 10, 20, 30, 40, 50, 60, 70, 80, 90, 100, 125, 150, 200, 225, 250, 275, 300, 350, 400, 450, or 500-fold higher than the level of expression of an phosphomevalonate kinase RNA molecule, diphosphomevalonate decarboxylase RNA molecule, and isopentenyl-diphosphate delta-isomerase RNA molecule. In some embodiments, the total amount of mevalonate kinase RNA is similar to the total amount of isoprene synthase RNA. For example, in some embodiments, the total amount of mevalonate kinase RNA is within about any of 10, 8, 6, 4, 2, 1, or 0.5-fold higher or lower than the total amount of isoprene synthase RNA (e.g., the amount of mevalonate kinase RNA may be between about 10-fold lower to about 10-fold higher than the amount of isoprene synthase RNA).

In some embodiments of any of the compositions, systems, and methods of the invention, the number of copies of a mevalonate kinase DNA molecule and/or an isoprene synthase DNA molecule is at least about any of 2, 5, 10, 20, 30, 40, 50, 60, 70, 80, 90, 100, 125, 150, 200, 225, 250, 275, 300, 350, 400, 450, or 500-fold (i) higher than the number of copies of a second MVA pathway DNA molecule (such as an acetyl-CoA acetyltransferase DNA molecule, 3-hydroxy-3-methylglutaryl-CoA synthase DNA molecule, 3-hydroxy-3-methylglutaryl-CoA reductase DNA molecule, phosphomevalonate kinase DNA molecule, diphosphomevalonate decarboxylase DNA molecule, or isopentenyl-diphosphate delta-isomerase DNA molecule) or (ii) higher than the number of copies of all other MVA pathway DNA molecules in the cell. In particular embodiments, the number of copies of a mevalonate kinase DNA molecule and/or an isoprene synthase DNA molecule is at least about any of 2, 5, 10, 12, 14, 16, 18, 20, 30, 40, 50, 60, 70, 80, 90, 100, 125, 150, 200, 225, 250, 275, 300, 350, 400, 450, or 500-fold higher than the number of copies of an acetyl-CoA acetyltransferase DNA molecule, 3-hydroxy-3-methylglutaryl-CoA synthase DNA molecule, and 3-hydroxy-3-methylglutaryl-CoA reductase DNA molecule. In particular embodiments, the number of copies of a mevalonate kinase DNA molecule and/or an isoprene synthase DNA molecule is at least about any of 2, 5, 10, 20, 30, 40, 50, 60, 70, 80, 90, 100, 125, 150, 200, 225, 250, 275, 300, 350, 400, 450, or 500-fold higher than the number of copies of an phosphomevalonate kinase DNA molecule, diphosphomevalonate decarboxylase DNA molecule, and isopentenyl-diphosphate delta-isomerase DNA molecule. In some embodiments, the number of copies of a mevalonate kinase DNA molecule is similar to the number of copies of an isoprene synthase DNA molecule. For example, in some embodiments, the number of copies of a mevalonate kinase DNA molecule is within about any of 10, 8, 6, 4, 2, 1, or 0.5-fold higher or lower than the number of copies of an isoprene synthase DNA molecule (e.g., the number of copies of a mevalonate kinase DNA may be between about 10-fold lower to about 10-fold higher than the number of copies of an isoprene synthase DNA molecule).

In some embodiments of any of the compositions, systems, and methods of the invention, the intracellular concentration of DMAPP is between about 0 to about 25 μmol/g_{dcw}, such as between about 0.1 to about 20 μmol/g_{dcw}, about 0.1 to about 15 μmol/g_{dcw}, about 0.1 to about 11 μmol/g_{dcw}, about 0.1 to about 7 μmol/g_{dcw}, about 0.1 to about 5 μmol/g_{dcw}, about 0.1 to about 2 μmol/g_{dcw}, about 0.1 to about 1 μmol/g_{dcw}, about 0.1 to about 0.8 μmol/g_{dcw}, about 0.1 to about 0.6 μmol/g_{dcw}, about 0.2 to about 15 μmol/g_{dcw}, about 0.2 to about 11 μmol/g_{dcw}, about 0.2 to about 7 μmol/g_{dcw}, about 0.2 to about 5 μmol/g_{dcw}, about 0.2 to about 2 μmol/g_{dcw}, about 0.3 to about 11 μmol/g_{dcw}, about 0.3 to about 7 μmol/g_{dcw}, about 0.3 to about 5 μmol/g_{dcw}, about 0.3 to about 2 μmol/g_{dcw}, about 0.3 to about 1 μmol/g_{dcw}, about 0.4 to about 11 μmol/g_{dcw}, about 0.4 to about 7 μmol/g_{dcw}, about 0.4 to about 5 μmol/g_{dcw}, about 0.4 to about 2 μmol/g_{dcw}, about 0.5 to about 7 μmol/g_{dcw}, about 0.5 to about 5 μmol/g_{dcw}, or about 0.5 to about 2 μmol/g_{dcw}. In some embodiments, the intracellular concentration of DMAPP is equal to or less than about any of 25, 20, 18, 16, 14, 12, 10, 8, 6, 4, 2, 1, 0.8, 0.6, 0.5, 0.4, 0.3, 0.2, or 0.1 μmol/g_{dcw}.

In some embodiments of any of the compositions, systems, and methods of the invention, the intracellular concentration of IPP is between about 0 to about 60 μmol/g_{dcw}, such as between about 0.1 to about 50 μmol/g_{dcw}, about 0.1 to about 40 μmol/g_{dcw}, about 0.1 to about 30 μmol/g_{dcw}, about 0.1 to about 20 μmol/g_{dcw}, about 0.1 to about 15 μmol/g_{dcw}, about 0.1 to about 11 μmol/g_{dcw}, about 0.1 to about 7 μmol/g_{dcw}, about 0.1 to about 5 μmol/g_{dcw}, about 0.1 to about 2 μmol/g_{dcw}, about 0.1 to about 1 μmol/g_{dcw}, about 0.1 to about 0.8 μmol/g_{dcw}, about 0.1 to about 0.6 μmol/g_{dcw}, about 0.2 to about 60 μmol/g_{dcw}, about 0.2 to about 50 μmol/g_{dcw}, about 0.2 to about 40 μmol/g_{dcw}, about 0.2 to about 30 μmol/g_{dcw}, about 0.2 to about 20 μmol/g_{dcw}, about 0.2 to about 15 μmol/g_{dcw}, about 0.2 to about 11 μmol/g_{dcw}, about 0.2 to about 7 μmol/g_{dcw}, about 0.2 to about 5 μmol/g_{dcw}, about 0.2 to about 2 μmol/g_{dcw}, about 0.3 to about 60 μmol/g_{dcw}, about 0.3 to about 50 μmol/g_{dcw}, about 0.3 to about 40 μmol/g_{dcw}, about 0.3 to about 30 μmol/g_{dcw}, about 0.3 to about 15 μmol/g_{dcw}, about 0.3 to about 11 μmol/g_{dcw}, about 0.3 to about 7 μmol/g_{dcw}, about 0.3 to about 5 μmol/g_{dcw}, about 0.3 to about 2 μmol/g_{dcw}, about 0.4 to about 60 μmol/g_{dcw}, about 0.4 to about 50 μmol/g_{dcw}, about 0.4 to about 40 μmol/g_{dcw}, about 0.4 to about 30 μmol/g_{dcw}, about 0.4 to about 15 μmol/g_{dcw}, about 0.4 to about 7 μmol/g_{dcw}, about 0.4 to about 5 μmol/g_{dcw}, about 0.4 to about 2 μmol/g_{dcw}, about 0.5 to about 60 μmol/g_{dcw}, about 0.5 to about 50 μmol/g_{dcw}, about 0.5 to about 40 μmol/g_{dcw}, about 0.5 to about 30 μmol/g_{dcw}, about 0.5 to about 15 μmol/g_{dcw}, about 0.5 to about 11 μmol/g_{dcw}, about 0.5 to about 7 μmol/g_{dcw}, about 0.5 to about 5 μmol/g_{dcw}, or about 0.5 to about 2 μmol/g_{dcw}. In some embodiments, the intracellular concentration of IPP is equal to or less than about any of 60, 50, 40, 30, 25, 20, 18, 16, 14, 12, 10, 8, 6, 4, 2, 1, 0.8, 0.6, 0.5, 0.4, 0.3, 0.2, or 0.1 μmol/g_{dcw}.

In some embodiments of any of the compositions, systems, and methods of the invention, the intracellular concentration of GPP is between about 0 to about 8 μmol/g_{dcw}, such as between about 0.1 to about 7 μmol/g_{dcw}, about 0.1 to about 6 μmol/g_{dcw}, about 0.1 to about 5 μmol/g_{dcw}, about 0.1 to about 4 μmol/g_{dcw}, about 0.1 to about 3 μmol/g_{dcw}, about 0.1 to about 2 μmol/g_{dcw}, about 0.1 to about 1 μmol/g_{dcw}, about 0.1 to about 0.8 μmol/g_{dcw}, about 0.1 to about 0.6 μmol/g_{dcw}, about 0.2 to about 7 μmol/g_{dcw}, about 0.2 to about 6 μmol/g_{dcw}, about 0.2 to about 5 μmol/g_{dcw}, about 0.2 to about 4 μmol/g_{dcw}, about 0.2 to about 3 μmol/g_{dcw}, about 0.2 to about 2 μmol/g_{dcw}, about 0.3 to about 7 μmol/g_{dcw}, about 0.3 to about 6 μmol/g_{dcw}, about 0.3 to about 5 μmol/g_{dcw}, about 0.3 to about 4 μmol/g_{dcw}, about 0.3 to about 3 μmol/g_{dcw}, about 0.3 to about 2 μmol/g_{dcw}, about 0.4 to about 7 μmol/g_{dcw}, about 0.4 to about 6 μmol/g_{dcw}, about 0.4 to about 5 μmol/g_{dcw}, about 0.4 to about 2 μmol/g_{dcw}, about 0.5 to about 7 μmol/g_{dcw}, about 0.5 to about 5 μmol/g_{dcw}, about 0.5 to about 2 μmol/g_{dcw}, about 0.6 to about 7 μmol/g_{dcw}, about 0.6 to about 5 μmol/g_{dcw}, about 0.6 to about 2 μmol/g_{dcw}, about 0.7 to about 7 μmol/g_{dcw}, about 0.7 to about 5 μmol/g_{dcw}, or about 0.7 to about 2 μmol/g_{dcw}. In some embodiments, the intracellular concentration of GPP is equal to or less than about any of 8, 6, 4, 2, 1, 0.8, 0.6, 0.5, 0.4, 0.3, 0.2, or 0.1 μmol/g_{dcw}.

In some embodiments of any of the compositions, systems, and methods of the invention, the intracellular concentration of FPP is between about 0 to about 6 μmol/g_{dcw}, such as between about 0. 1 to about 6 μmol/g_{dcw}, about 0.1 to about 5 μmol/g_{dcw}, about 0.1 to about 4 μmol/g_{dcw}, about 0.1 to about 3 μmol/g_{dcw}, about 0.1 to about 2 μmol/g_{dcw}, about 0.1 to about 1 μmol/g_{dcw}, about 0.1 to about 0.8 μmol/g_{dcw}, about 0.1 to about 0.6 μmol/g_{dcw}, about 0.2 to about 6 μmol/g_{dcw}, about 0.2 to about 5 μmol/g_{dcw}, about 0.2 to about 4 μmol/g_{dcw}, about 0.2 to about 3 μmol/g_{dcw}, about 0.2 to about 2 μmol/g_{dcw}, about 0.3 to about 6 μmol/g_{dcw}, about 0.3 to about 5 μmol/g_{dcw}, about 0.3 to about 4 μmol/g_{dcw}, about 0.3 to about 3 μmol/g_{dcw}, about 0.3 to about 2 μmol/g_{dcw}, about 0.4 to about 6 μmol/g_{dcw}, about 0.4 to about 5 μmol/g_{dcw}, about 0.4 to about 2 μmol/g_{dcw}, about 0.5 to about 6 μmol/g_{dcw}, about 0.5 to about 5 μmol/g_{dcw}, about 0.5 to about 2 μmol/g_{dcw}, about 0.8 to about 6 μmol/g_{dcw}, about 0.8 to about 5 μmol/g_{dcw}, about 0.8 to about 2 μmol/g_{dcw}, about 1 to about 6 μmol/g_{dcw}, about 1 to about 5 μmol/g_{dcw}, about 1 to about 2 μmol/g_{dcw}, about 1.1 to about 6 μmol/g_{dcw}, about 1.1 to about 5 μmol/g_{dcw}, about 1.1 to about 2 μmol/g_{dcw}, about 1.1 to about 1.5 μmol/g_{dcw}, about 1.2 to about 6 μmol/g_{dcw}, about 1.2 to about 5 μmol/g_{dcw}, about 1.2 to about 2 μmol/g_{dcw}, or about 1.2 to about 1.5 μmol/g_{dcw}. In some embodiments, the intracellular concentration of FPP is equal to or less than about any of 6, 4, 2, 1.5, 1.4, 1.3, 1.2, 1.1, 1, 0.9, 0.8, 0.6, 0.5, 0.4, 0.3, 0.2, or 0.1 μmol/g_{dcw}.

In some embodiments of any of the compositions, systems, and methods of the invention, the concentration (e.g., concentration in the cell medium) of MVA is between about 0 to about 120 g/L, such as between about about 0 to about 110 g/L, such as between about 0.1 to about 100 g/L, about 0.1 to about 75 g/L, about 0.1 to about 60 g/L, about 0.1 to about 50 g/L, about 0.1 to about 40 g/L, about 0.1 to about 30 g/L, about 0.1 to about 20 g/L, about 0. 1 to about 15 g/L, about 0.1 to about 11 g/L, about 0.1 to about 7 g/L, about 0.1 to about 5 g/L, about 0.1 to about 2 g/L, about 0.1 to about 1 g/L, about 0.1 to about 0.8 g/L, about 0.1 to about 0.6 g/L, about 0.2 to about 120 g/L, about 0.2 to about 100 g/L, about 0.2 to about 75 g/L, about 0.2 to about 60 g/L, about 0.2 to about 50 g/L, about 0.2 to about 40 g/L, about 0.2 to about 30 g/L, about 0.2 to about 20 g/L, about 0.2 to about 15 g/L, about 0.2 to about 11 g/L, about 0.2 to about 7 g/L, about 0.2 to about 5 g/L, about 0.2 to about 2 g/L, about 0.3 to about 120 g/L, about 0.3 to about 100 g/L, about 0.3 to about 75 g/L, about 0.3 to about 60 g/L, about 0.3 to about 50 g/L, about 0.3 to about 40 g/L, about 0.3 to about 30 g/L, about 0.3 to about 15 g/L, about 0.3 to about 11 g/L, about 0.3 to about 7 g/L, about 0.3 to about 5 g/L, about 0.3 to about 2 g/L, about 0.4 to about 120 g/L, about 0.4 to about 100 g/L, about 0.4 to about 75 g/L, about 0.4 to about 60 g/L, about 0.4 to about 50 g/L, about 0.4 to about 40 g/L, about 0.4 to about 30 g/L, about 0.4 to about 15 g/L, about 0.4 to about 7 g/L, about 0.4 to about 5 g/L, about 0.4 to about 2 g/L, about 0.5 to about 1200 g/L, about 0.5 to about 100 g/L, about 0.5 to about 75 g/L, about 0.5 to about 60 g/L, about 0.5 to about 50 g/L, about 0.5 to about 40 g/L, about 0.5 to about 30 g/L, about 0.5 to about 15 g/L, about 0.5 to about 11 g/L, about 0.5 to about 7 g/L, about 0.5 to about 5 g/L, about 0.5 to about 2 g/L, about 50 to about 60 g/L, or about 1 g/L. In some embodiments, the concentration (e.g., concentration in the cell medium) of MVA is equal to or less than about any of 120, 100, 80, 70, 60, 50, 40, 30, 25, 20, 18, 16, 14, 12, 10, 8, 6, 4, 2, 1, 0.8, 0.6, 0.5, 0.4, 0.3, 0.2, or 0.1 g/L.

In some embodiments of any of the compositions, systems, and methods of the invention, the cells comprise a heterologous nucleic acid or a duplicate copy of an endogenous nucleic acid encoding a mevalonate kinase polypeptide. In some embodiments, the mevalonate kinase nucleic acid is operably linked to a promoter. In some embodiments, the cells express (i) a heterologous nucleic acid encoding a second mevalonate kinase polypeptide or (ii) a duplicate copy of a nucleic acid encoding a second mevalonate kinase polypeptide that differs from the first mevalonate kinase polypeptide. In some embodiments, the cells comprise a heterologous nucleic acid or a duplicate copy of an endogenous nucleic acid encoding an isoprene synthase polypeptide. In some embodiments, the cells have a heterologous nucleic acid that (i) encodes an isoprene synthase polypeptide and (ii) is operably linked to a promoter.

In some embodiments, isoprene is only produced in stationary phase. In some embodiments, isoprene is produced in both the growth phase and stationary phase. In various embodiments, the amount of isoprene produced (such as the total amount of isoprene produced or the amount of isoprene produced per liter of broth per hour per OD₆₀₀) during stationary phase is greater than or about 2, 3, 4, 5, 10, 20, 30, 40, 50, or more times the amount of isoprene produced during the growth phase for the same length of time.

In some embodiments, at least a portion of the isoprene is in a gas phase. In some embodiments, at least a portion of the isoprene is in a liquid phase (such as a condensate). In some embodiments, at least a portion of the isoprene is in a solid phase. In some embodiments, at least a portion of the isoprene is adsorbed to a solid support, such as a support that includes silica and/or activated carbon. In some embodiments, the composition includes ethanol. In some embodiments, the composition includes between about 75 to about 90% by weight of ethanol, such as between about 75 to about 80%, about 80 to about 85%, or about 85 to about 90% by weight of ethanol. In some embodiments, the composition includes between about 4 to about 15% by weight of isoprene, such as between about 4 to about 8%, about 8 to about 12%, or about 12 to about 15% by weight of isoprene.

In some embodiments, the invention also features systems that include any of the cells and/or compositions described herein. In some embodiments, the system includes a reactor that chamber comprises cells in culture that produce greater than about 400, 500, 600, 700, 800, 900, 1,000, 1,250, 1,500, 1,750, 2,000, 2,500, 3,000, 4,000, 5,000, or more nmole/g_{wcm}/hr isoprene. In some embodiments, the system is not a closed system. In some embodiments, at least a portion of the isoprene is removed from the system. In some embodiments, the system includes a gas phase comprising isoprene. In various embodiments, the gas phase comprises any of the compositions described herein.

In one aspect, the invention provides a tire comprising polyisoprene. In some embodiments, the polyisoprene is produced by (i) polymerizing isoprene in any of the compositions described herein or (ii) polymerizing isoprene recovered from any of the compositions described herein. In some embodiments, the polyisoprene comprises cis-1,4-polyisoprene. In another aspect, the invention provides methods of manufacturing a tire wherein the improvement comprises using any one or more the compositions, cells, systems and/or methods described herein to produce isoprene for the manufacture of the tire.

In some embodiments of any of the compositions, systems, and methods of the invention, a nonflammable concentration of isoprene in the gas phase is produced. In some embodiments, the gas phase comprises less than about 9.5% (volume) oxygen. In some embodiments, the gas phase comprises greater than or about 9.5% (volume) oxygen, and the concentration of isoprene in the gas phase is less than the lower flammability limit or greater than the upper flammability limit. In some embodiments, the portion of the gas phase other than isoprene comprises between about 0% to about 100% (volume) oxygen, such as between about 10% to about 100% (volume) oxygen. In some embodiments, the portion of the gas phase other than isoprene comprises between about 0% to about 99% (volume) nitrogen. In some embodiments, the portion of the gas phase other than isoprene comprises between about 1% to about 50% (volume) CO₂.

In some embodiments of any of the aspects of the invention, the cells in culture produce isoprene at greater than or about 400, 500, 600, 700, 800, 900, 1,000, 1,250, 1,500, 1,750, 2,000, 2,500, 3,000, 4,000, 5,000, or more nmole/g_{wcm}/hr isoprene. In some embodiments of any of the aspects of the invention, the cells in culture convert greater than or about 0.002, 0.005, 0.01, 0.02, 0.05, 0.1, 0.12, 0.14, 0.16, 0.2, 0.3, 0.4, 0.5, 0.6, 0.7, 0.8, 0.9, 1.0, 1.2, 1.4, 1.6%, or more of the carbon in the cell culture medium into isoprene. In some embodiments of any of the aspects of the invention, the cells in culture produce isoprene at greater than or about 1, 10, 25, 50, 100, 150, 200, 250, 300, 400, 500, 600, 700, 800, 900, 1,000, 1,250, 1,500, 1,750, 2,000, 2,500, 3,000, 4,000, 5,000, 10,000, 100,000, or more ng of isoprene/gram of cells for the wet weight of the cells/hr (ng/g_{wcm}/h). In some embodiments of any of the aspects of the invention, the cells in culture produce a cumulative titer (total amount) of isoprene at greater than or about 1, 10, 25, 50, 100, 150, 200, 250, 300, 400, 500, 600, 700, 800, 900, 1,000, 1,250, 1,500, 1,750, 2,000, 2,500, 3,000, 4,000, 5,000, 10,000, 50,000, 100,000, or more mg of isoprene/L of broth (mg/L_{broth}, wherein the volume of broth includes the volume of the cells and the cell medium). Other exemplary rates of isoprene production and total amounts of isoprene production are disclosed herein.

In some embodiments of any of the aspects of the invention, the cells further comprise a heterologous nucleic acid encoding an IDI polypeptide. In some embodiments of any of the aspects of the invention, the cells further comprise an insertion of a copy of an endogenous nucleic acid encoding an IDI polypeptide. In some embodiments of any of the aspects of the invention, the cells further comprise a heterologous nucleic acid encoding a DXS polypeptide. In some embodiments of any of the aspects of the invention, the cells further comprise an insertion of a copy of an endogenous nucleic acid encoding a DXS polypeptide. In some embodiments of any of the aspects of the invention, the cells further comprise one or more nucleic acids encoding an IDI polypeptide and a DXS polypeptide. In some embodiments of any of the aspects of the invention, one nucleic acid encodes the isoprene synthase polypeptide, IDI polypeptide, and DXS polypeptide. In some embodiments of any of the aspects of the invention, one vector encodes the isoprene synthase polypeptide, IDI polypeptide, and DXS polypeptide. In some embodiments, the vector comprises a selective marker, such as an antibiotic resistance nucleic acid.

In some embodiments of any of the aspects of the invention, the cells further comprise a heterologous nucleic acid encoding an MVA pathway polypeptide (such as an MVA pathway polypeptide from Saccharomyces cerevisia or Enterococcus faecalis). In some embodiments of any of the aspects of the invention, the cells further comprise an insertion of a copy of an endogenous nucleic acid encoding an MVA pathway polypeptide (such as an MVA pathway polypeptide from Saccharomyces cerevisia or Enterococcus faecalis). In some embodiments of any of the aspects of the invention, the cells comprise an isoprene synthase, DXS, and MVA pathway nucleic acid. In some embodiments of any of the aspects of the invention, the cells comprise an isoprene synthase nucleic acid, a DXS nucleic acid, an IDI nucleic acid, and a MVA pathway nucleic (in addition to the IDI nucleic acid).

In some embodiments of any of the aspects of the invention, the isoprene synthase polypeptide is a polypeptide from a plant such as Pueraria (e.g., Pueraria montana or Pueraria lobata) or Populus (e.g., Populus tremuloides, Populus alba, Populus nigra, Populus trichocarpa, or the hybrid, Populus alba x Populus tremula).

In some embodiments, one or more MVA pathway, IDI, DXP, or isoprene synthase nucleic acids are placed under the control of a promoter or factor that is more active in stationary phase than in the growth phase. For example, one or more MVA pathway, IDI, DXP, or isoprene synthase nucleic acids may be placed under control of a stationary phase sigma factor, such as RpoS. In some embodiments, one or more MVA pathway, IDI, DXP, or isoprene synthase nucleic acids are placed under control of a promoter inducible in stationary phase, such as a promoter inducible by a response regulator active in stationary phase.

In some embodiments of any of the aspects of the invention, the cells are bacterial cells, such as gram-positive bacterial cells (e.g., Bacillus cells such as Bacillus subtilis cells or Streptomyces cells such as Streptomyces lividans, Streptomyces coelicolor, or Streptomyces griseus cells). In some embodiments of any of the aspects of the invention, the cells are gram-negative bacterial cells (e.g., Escherichia cells such as Escherichia coli cells or Pantoea cells such as Pantoea citrea cells). In some embodiments of any of the aspects of the invention, the cells are fungal, cells such as filamentous fungal cells (e.g., Trichoderma cells such as Trichoderma reesei cells or Aspergillus cells such as Aspergillus oryzae and Aspergillus niger) or yeast cells (e.g., Yarrowia cells such as Yarrowia lipolytica cells or Saccharomyces cells such as Saccharomyces cerevisiae).

In some embodiments of any of the aspects of the invention, the microbial polypeptide carbon source includes one or more polypeptides from yeast or bacteria. In some embodiments of any of the aspects of the invention, the plant polypeptide carbon source includes one or more polypeptides from soy, corn, canola, jatropha, palm, peanut, sunflower, coconut, mustard, rapeseed, cottonseed, palm kernel, olive, safflower, sesame, or linseed.

In one aspect, the invention features a product produced by any of the compositions or methods of the invention.

It is to be understood that one, some, or all of the properties of the various embodiments described herein may be combined to form other embodiments of the present invention.

### BRIEF DESCRIPTION OF THE DRAWINGS

FIG. 1 is the nucleotide sequence of a kudzu isoprene synthase gene codon-optimized for expression in E. coli (SEQ ID NO:1). The atg start codon is in italics, the stop codon is in bold and the added Pstl site is underlined.

FIG. 2 is a map of pTrcKudzu.

FIGS. 3A-3C are the nucleotide sequence of pTrcKudzu (SEQ ID NO:2). The RBS is underlined, the kudzu isoprene synthase start codon is in bold capitol letters and the stop codon is in bold, capitol, italics letters. The vector backbone is pTrcHis2B.

FIG. 4 is a map of pETNHisKudzu.

FIGS. 5A-5C are the nucleotide sequence of pETNHisKudzu (SEQ ID NO:5).

FIG. 6 is a map of pCL-lac-Kudzu.

FIGS. 7A-7C are the nucleotide sequence of pCL-lac-Kudzu (SEQ ID NO:7).

FIG. 8A is a graph showing the production of isoprene in E. coli BL21 cells with no vector.

FIG. 8B is a graph showing the production of isoprene in E. coli BL21 cells with pCL-lac-Kudzu

FIG. 8C is a graph showing the production of isoprene in E. coli BL21 cells with pTrcKudzu.

FIG. 8D is a graph showing the production of isoprene in E. coli BL21 cells with pETN-HisKudzu.

FIG. 9A is a graph showing OD over time of fermentation of E. coli BL21/pTrcKudzu in a 14 liter fed batch fermentation.

FIG. 9B is a graph showing isoprene production over time of fermentation of E. coli BL21/pTrcKudzu in a 14 liter fed batch fermentation.

FIG. 10A is a graph showing the production of isoprene in Panteoa citrea. Control cells without recombinant kudzu isoprene synthase. Grey diamonds represent isoprene synthesis, black squares represent OD₆₀₀.

FIG. 10B is a graph showing the production of isoprene in Panteoa citrea expressing pCL-lac Kudzu. Grey diamonds represent isoprene synthesis, black squares represent OD₆₀₀.

FIG. 10C is a graph showing the production of isoprene in Panteoa citrea expressing pTrcKudzu. Grey diamonds represent isoprene synthesis, black squares represent OD₆₀₀.

FIG. 11 is a graph showing the production of isoprene in Bacillus subtilis expressing recombinant isoprene synthase. BG3594comK is a B. subtilis strain without plasmid (native isoprene production). CF443-BG3594comK is a B. subtilis strain with pBSKudzu (recombinant isoprene production). IS on the y-axis indicates isoprene.

FIGS. 12A-12C are the nucleotide sequence of pBS Kudzu #2 (SEQ ID NO:57).

FIG. 13 is the nucleotide sequence of kudzu isoprene synthase codon-optimized for expression in Yarrowia (SEQ ID NO:8).

FIG. 14 is a map of pTrex3g comprising a kudzu isoprene synthase gene codon-optimized for expression in Yarrowia.

FIGS. 15A-15C are the nucleotide sequence of vector pSPZ1(MAP29Spb) (SEQ ID NO:11).

FIG. 16 is the nucleotide sequence of the synthetic kudzu (Pueraria montana) isoprene gene codon-optimized for expression in Yarrowia (SEQ ID NO:12).

FIG. 17 is the nucleotide sequence of the synthetic hybrid poplar (Populus alba x Populus tremula) isoprene synthase gene (SEQ ID NO:13). The ATG start codon is in bold and the stop codon is underlined.

FIG. 18A shows a schematic outlining construction of vectors pYLA 1, pYL1 and pYL2.

FIG. 18B shows a schematic outlining construction of the vector pYLA(POP1).

FIG. 18C shows a schematic outlining construction of the vector pYLA(KZ1)

FIG. 18D shows a schematic outlining construction of the vector pYLI(KZ1)

FIG. 18E shows a schematic outlining construction of the vector pYLI(MAP29)

FIG. 18F shows a schematic outlining construction of the vector pYLA(MAP29)

FIG. 19A shows the MVA and DXP metabolic pathways for isoprene (based on F. Bouvier et al., Progress in Lipid Res. 44: 357-429, 2005). The following description includes alternative names for each polypeptide in the pathways and a reference that discloses an assay for measuring the activity of the indicated polypeptide (each of these references are each hereby incorporated by reference in their entireties, particularly with respect to assays for polypeptide activity for polypeptides in the MVA and DXP pathways). Mevalonate Pathway: AACT; Acetyl-CoA acetyltransferase, MvaE, EC 2.3.1.9. Assay: J. Bacteriol., 184: 2116-2122, 2002; HMGS; Hydroxymethylglutaryl-CoA synthase, MvaS, EC 2.3.3.10. Assay: J. Bacteriol., 184: 4065-4070, 2002; HMGR; 3-Hydroxy-3-methylglutaryl-CoA reductase, MvaE, EC 1.1.1.34. Assay: J. Bacteriol., 184: 2116-2122, 2002; MVK; Mevalonate kinase, ERG12, EC 2.7.1.36. Assay: Curr Genet 19:9-14, 1991. PMK; Phosphomevalonate kinase, ERGS, EC 2.7.4.2, Assay: Mol Cell Biol., 11:620-631, 1991; DPMDC; Diphosphomevalonate decarboxylase, MVD1, EC 4.1.1.33. Assay: Biochemistry, 33:13355-13362, 1994; IDI; Isopentenyl-diphosphate delta-isomerase, IDI1, EC 5.3.3.2. Assay: J. Biol. Chem. 264:19169-19175, 1989. DXP Pathway: DXS; 1-Deoxyxylulose-5-phosphate synthase, dxs, EC 2.2.1.7. Assay: PNAS, 94:12857-62, 1997; DXR; 1-Deoxy-D-xylulose 5-phosphate reductoisomerase, dxr, EC 2.2.1.7. Assay: Eur. J. Biochem. 269:4446-4457, 2002; MCT; 4-Diphosphocytidyl-2C-methyl-D-erythritol synthase, IspD, EC 2.7.7.60. Assay: PNAS, 97: 6451-6456, 2000; CMK; 4-Diphosphocytidyl-2-C-methyl-D-erythritol kinase, IspE, EC 2.7.1.148. Assay: PNAS, 97:1062-1067, 2000; MCS; 2C-Methyl-D-erythritol 2,4-cyclodiphosphate synthase, IspF, EC 4.6.1.12. Assay: PNAS, 96:11758-11763, 1999; HDS; 1-Hydroxy-2-methyl-2-(E)-butenyl 4-diphosphate synthase, ispG, EC 1.17.4.3. Assay: J. Org. Chem., 70:9168 -9174, 2005; HDR; 1-Hydroxy-2-methyl-2-(E)-butenyl 4-diphosphate reductase, IspH, EC 1.17.1.2. Assay: JACS, 126:12847-12855, 2004.

FIG. 19B illustrates the classical and modified MVA pathways. 1, acetyl-CoA acetyltransferase (AACT); 2, HMG-CoA synthase (HMGS); 3, HMG-CoA reductase (HMGR); 4, mevalonate kinase (MVK); 5, phosphomevalonate kinase (PMK); 6, diphosphomevalonate decarboxylase (MVD or DPMDC); 7, isopentenyl diphosphate isomerase (IDI); 8, phosphomevalonate decarboxylase (PMDC); 9, isopentenyl phosphate kinase (IPK). The classical MVA pathway proceeds from reaction 1 through reaction 7 via reactions 5 and 6, while a modified MVA pathway goes through reactions 8 and 9. P and PP in the structural formula are phosphate and pyrophosphate, respectively. This figure was taken from Koga and Morii, Microbiology and Mol. Biology Reviews, 71:97-120, 2007, which is incorporated by reference in its entirety, particularly with respect to nucleic acids and polypeptides of the modified MVA pathway. The modified MVA pathway is present, for example, in some archaeal organisms, such as Methanosarcina mazei.

FIG. 20 shows graphs representing results of the GC-MS analysis of isoprene production by recombinant Y. lipolytica strains without (left) or with (right) a kudzu isoprene synthase gene. The arrows indicate the elution time of the authentic isoprene standard.

FIG. 21 is a map of pTrcKudzu yIDI DXS Kan.

FIGS. 22A-22D are the nucleotide sequence of pTrcKudzu yIDI DXS Kan (SEQ ID NO:20).

FIG. 23A is a graph showing production of isoprene from glucose in BL21/pTrcKudzukan. Time 0 is the time of induction with IPTG (400 μmol). The x-axis is time after induction; the y-axis is OD₆₀₀ and the y2-axis is total productivity of isoprene (μg/L headspace or specific productivity (μg/L headspace/OD). Diamonds represent OD₆₀₀, circles represent total isoprene productivity (μg/L) and squares represent specific productivity of isoprene (μg/L/OD).

FIG. 23B is a graph showing production of isoprene from glucose in BL21/pTrcKudzu yIDI kan. Time 0 is the time of induction with IPTG (400 μmol). The x-axis is time after induction; the y-axis is OD₆₀₀ and the y2-axis is total productivity of isoprene (μg/L headspace or specific productivity (μg/L headspace/OD). Diamonds represent OD₆₀₀, circles represent total isoprene productivity (μg/L) and squares represent specific productivity of isoprene (μg/L/OD).

FIG. 23C is a graph showing production of isoprene from glucose in BL21/pTrcKudzu DXS kan. Time 0 is the time of induction with IPTG (400 μmol). The x-axis is time after induction; the y-axis is OD₆₀₀ and the y2-axis is total productivity of isoprene (μg/L headspace or specific productivity (μg/L headspace/OD). Diamonds represent OD₆₀₀, circles represent total isoprene productivity (μg/L) and squares represent specific productivity of isoprene (μg/L/OD).

FIG. 23D is a graph showing production of isoprene from glucose in BL21/pTrcKudzu yIDI DXS kan. Time 0 is the time of induction with IPTG (400 μmol). The x-axis is time after induction; the y-axis is OD₆₀₀ and the y2-axis is total productivity of isoprene (μg/L headspace or specific productivity (μg/L headspace/OD). Diamonds represent OD₆₀₀, circles represent total isoprene productivity (μg/L) and squares represent specific productivity of isoprene (μg/L/OD).

FIG. 23E is a graph showing production of isoprene from glucose in BL21/pCL PtrcKudzu. Time 0 is the time of induction with IPTG (400 μmol). The x-axis is time after induction; the y-axis is OD₆₀₀ and the y2-axis is total productivity of isoprene (μg/L headspace or specific productivity (μg/L headspace/OD). Diamonds represent OD₆₀₀, circles represent total isoprene productivity (μg/L) and squares represent specific productivity of isoprene (μg/L/OD).

FIG. 23F is a graph showing production of isoprene from glucose in BL21/pCL PtrcKudzu yIDI. Time 0 is the time of induction with IPTG (400 μmol). The x-axis is time after induction; the y-axis is OD₆₀₀ and the y2-axis is total productivity of isoprene (μg/L headspace or specific productivity (μg/L headspace/OD). Diamonds represent OD₆₀₀, circles represent total isoprene productivity (μg/L) and squares represent specific productivity of isoprene (μg/L/OD).

FIG. 23G is a graph showing production of isoprene from glucose in BL21/pCL PtrcKudzu DXS. Time 0 is the time of induction with IPTG (400 μmol). The x-axis is time after induction; the y-axis is OD₆₀₀ and the y2-axis is total productivity of isoprene (μg/L headspace or specific productivity (μg/L headspace/OD). Diamonds represent OD₆₀₀, circles represent total isoprene productivity (μg/L) and squares represent specific productivity of isoprene (μg/L/OD).

FIG. 23H is a graph showing production of isoprene from glucose in BL21/pTrcKudzuIDIDXSkan. The arrow indicates the time of induction with IPTG (400 μmol). The x-axis is time after inoculation; the y-axis is OD600 and the y2-axis is total productivity of isoprene (μg/L headspace or specific productivity (μg/L headspace/OD). Diamonds represent OD600, triangles represent total isoprene productivity (μg/L) and squares represent specific productivity of isoprene (μg/L/OD).

FIG. 24 is a map of pTrcKKDyIkIS kan.

FIGS. 25A-25D are the nucleotide sequence of pTrcKKDyIkIS kan (SEQ ID NO:33).

FIG. 26 is a map of pCL PtrcUpperPathway.

FIGS. 27A-27D are the nucleotide sequence of pCL PtrcUpperPathway (SEQ ID NO:46).

FIG. 28 shows a map of the cassette containing the lower MVA pathway and yeast idi for integration into the B. subtilis chromosome at the nprE locus. nprE upstream/downstream indicates 1 kb each of sequence from the nprE locus for integration. aprE promoter (alkaline serine protease promoter) indicates the promoter (−35, −10, +1 transcription start site, RBS) of the aprE gene. MVK1 indicates the yeast mevalonate kinase gene. RBS-PMK indicates the yeast phosphomevalonate kinase gene with a Bacillus RBS upstream of the start site. RBS-MPD indicates the yeast diphosphomevalonate decarboxylase gene with a Bacillus RBS upstream of the start site. RBS-IDI indicates the yeast idi gene with a Bacillus RBS upstream of the start site. Terminator indicates the terminator alkaline serine protease transcription terminator from B. amyliquefaciens. SpecR indicates the spectinomycin resistance marker. “nprE upstream repeat for amp.” indicates a direct repeat of the upstream region used for amplification.

FIGS. 29A-29D are the nucleotide sequence of cassette containing the lower MVA pathway and yeast idi for integration into the B. subtilis chromosome at the nprE locus (SEQ ID NO:47).

FIG. 30 is a map of p9796-poplar.

FIGS. 31A and 31B are the nucleotide sequence of p9796-poplar (SEQ ID NO:48).

FIG. 32 is a map of pTrcPoplar.

FIGS. 33A-33C are the nucleotide sequence of pTrcPoplar (SEQ ID NO:49).

FIG. 34 is a map of pTrcKudzu yIDI Kan.

FIGS. 35A-35C are the nucleotide sequence of pTrcKudzu yIDI Kan (SEQ ID NO:50).

FIG. 36 is a map of pTrcKudzuDXS Kan.

FIGS. 37A-37C are the nucleotide sequence of pTrcKudzuDXS Kan (SEQ ID NO:51).

FIG. 38 is a map of pCL PtrcKudzu.

FIGS. 39A-39C are the nucleotide sequence of pCL PtrcKudzu (SEQ ID NO:52).

FIG. 40 is a map of pCL PtrcKudzu A3.

FIGS. 41A-41C are the nucleotide sequence of pCL PtrcKudzu A3 (SEQ ID NO:53).

FIG. 42 is a map of pCL PtrcKudzu yIDI.

FIGS. 43A-43C are the nucleotide sequence of pCL PtrcKudzu yIDI (SEQ ID NO:54).

FIG. 44 is a map of pCL PtrcKudzu DXS.

FIGS. 45A-45D are the nucleotide sequence of pCL PtrcKudzu DXS (SEQ ID NO:55).

FIG. 46A is a map of the M. mazei archaeal Lower Pathway operon.

FIGS. 46B and 46C are the nucleotide sequence of the M. mazei archaeal lower Pathway operon (SEQ ID NO:102).

FIG. 47A is a map of MCM382-pTrcKudzuMVK(mazei).

FIGS. 47B and 47C are the nucleotide sequence of MCM382-pTrcKudzuMVK(mazei) (SEQ ID NO:103).

FIGS. 48A-48C are graphs demonstrating the effect of yeast extract of isoprene production. FIG. 48A is the time course of optical density within fermentors fed with varying amounts of yeast extract. FIG. 48B is the time course of isoprene titer within fermentors fed with varying amounts of yeast extract. The titer is defined as the amount of isoprene produced per liter of fermentation broth. FIG. 48C shows the effect of yeast extract on isoprene production in E. coli grown in fed-batch culture.

FIG. 49 shows graphs demonstrating isoprene production from a 500 L bioreactor with E. coli cells containing the pTrcKudzu+yIDI+DXS plasmid. Panel A shows the time course of optical density within the 500-L bioreactor fed with glucose and yeast extract. Panel B shows the time course of isoprene titer within the 500-L bioreactor fed with glucose and yeast extract. The titer is defined as the amount of isoprene produced per liter of fermentation broth. Panel C shows the time course of total isoprene produced from the 500-L bioreactor fed with glucose and yeast extract.

FIG. 50 is a map of pJMupperpathway2.

FIGS. 51A-51C are the nucleotide sequence of pJMupperpathway2 (SEQ ID NO:56).

FIG. 52 is a map of pBS Kudzu #2.

FIG. 53A is a graph showing growth during fermentation time of Bacillus expressing recombinant kudzu isoprene synthase in 14 liter fed batch fermentation. Black diamonds represent a control strain (BG3594comK) without recombinant isoprene synthase (native isoprene production) and grey triangles represent Bacillus with pBSKudzu (recombinant isoprene production).

FIG. 53B is a graph showing isoprene production during fermentation time of Bacillus expressing recombinant kudzu isoprene synthase in 14 liter fed batch fermentation. Black diamonds represent a control strain (BG3594comK) without recombinant isoprene synthase (native isoprene production) and grey triangles represent Bacillus with pBSKudzu (recombinant isoprene production).

FIG. 54 is a time course of optical density within the 15-L bioreactor fed with glucose.

FIG. 55 is a time course of isoprene titer within the 15-L bioreactor fed with glucose. The titer is defined as the amount of isoprene produced per liter of fermentation broth.

FIG. 56 is a time course of total isoprene produced from the 15-L bioreactor fed with glucose.

FIG. 57A is a map of MCM376-MVK from M. mazei archaeal Lower in pET200D.

FIGS. 57B and 57C are the nucleotide sequence of MCM376-MVK from M. mazei archaeal Lower in pET200D (SEQ ID NO:104).

FIG. 58A is a map of Streptomyces CL190 Lower Pathway Operon.

FIGS. 58B and 58C are the nucleotide sequence of Streptomyces CL190 Lower Pathway Operon (SEQ ID NO:105).

FIG. 59A is a map of MCM 383-pTrcKudzuMVK (S. cerevisiae).

FIGS. 59B and 59C are the nucleotide sequence of MCM 383-pTrcKudzuMVK (S. cerevisiae) (SEQ ID NO:106).

FIGS. 60A-60C are the time courses of optical density, mevalonic acid titer, and specific productivity within the 150-L bioreactor fed with glucose.

FIGS. 61A-61C are the time courses of optical density, mevalonic acid titer, and specific productivity within the 15-L bioreactor fed with glucose.

FIGS. 62A-62C are the time courses of optical density, mevalonic acid titer, and specific productivity within the 15-L bioreactor fed with glucose.

FIG. 63A-63C are the time courses of optical density, isoprene titer, and specific productivity within the 15-L bioreactor fed with glucose.

FIGS. 64A-64C are the time courses of optical density, isoprene titer, and specific productivity within the 15-L bioreactor fed with glucose.

FIGS. 65A-65C are the time courses of optical density, isoprene titer, and specific productivity within the 15-L bioreactor fed with glucose.

FIGS. 66A-66C are the time courses of optical density, isoprene titer, and specific productivity within the 15-L bioreactor fed with glucose.

FIG. 67A-67C are the time courses of optical density, isoprene titer, and specific productivity within the 15-L bioreactor fed with glucose.

FIG. 68 is a graph of the calculated adiabatic flame temperatures for Series A as a function of fuel concentration for various oxygen levels. The figure legend lists the curves in the order in which they appear in the graph. For example, the first entry in the figure legend (isoprene in air at 40° C.) corresponds to the highest curve in the graph.

FIG. 69 is a graph of the calculated adiabatic flame temperatures for Series B as a function of fuel concentration for various oxygen levels with 4% water. The figure legend lists the curves in the order in which they appear in the graph.

FIG. 70 is a graph of the calculated adiabatic flame temperatures for Series C as a function of fuel concentration for various oxygen levels with 5% CO₂. The figure legend lists the curves in the order in which they appear in the graph.

FIG. 71 is a graph of the calculated adiabatic flame temperatures for Series D as a function of fuel concentration for various oxygen levels with 10% CO₂. The figure legend lists the curves in the order in which they appear in the graph.

FIG. 72 is a graph of the calculated adiabatic flame temperatures for Series E as a function of fuel concentration for various oxygen levels with 15% CO₂. The figure legend lists the curves in the order in which they appear in the graph.

FIG. 73 is a graph of the calculated adiabatic flame temperatures for Series F as a function of fuel concentration for various oxygen levels with 20% CO₂. The figure legend lists the curves in the order in which they appear in the graph.

FIG. 74 is a graph of the calculated adiabatic flame temperatures for Series G as a function of fuel concentration for various oxygen levels with 30% CO₂. The figure legend lists the curves in the order in which they appear in the graph.

FIG. 75A is a table of the conversion of the CAFT Model results from weight percent to volume percent for series A.

FIG. 75B is a graph of the flammability results from the CAFT model for Series A in FIG. 68 plotted as volume percent.

FIG. 76A is a table of the conversion of the CAFT Model results from weight percent to volume percent for series B.

FIG. 76B is a graph of the flammability results from the CAFT model for Series B in FIG. 69 plotted as volume percent.

FIG. 77 is a figure of the flammability test vessel.

FIG. 78A is a graph of the flammability Curve for Test Series 1: 0% Steam, 0 psig, and 40° C.

FIG. 78B is a table summarizing the explosion and non-explosion data points for Test Series 1.

FIG. 78C is a graph of the flammability curve for Test Series 1 compared with the CAFT Model.

FIG. 79A is a graph of the flammability curve for Test Series 2: 4% Steam, 0 psig, and 40° C.

FIG. 79B is a table summarizing the explosion and non-explosion data points for Test Series 2.

FIG. 79C is a graph of the flammability curve for Test Series 2 compared with the CAFT Model.

FIGS. 80A and 80B are a table of the detailed experimental conditions and results for Test Series 1.

FIG. 81 is a table of the detailed experimental conditions and results for Test Series 2.

FIG. 82 is a graph of the calculated adiabatic flame temperature plotted as a function of fuel concentration for various nitrogen/oxygen ratios at 3 atmospheres of pressure.

FIG. 83 is a graph of the calculated adiabatic flame temperature plotted as a function of fuel concentration for various nitrogen/oxygen ratios at 1 atmosphere of pressure.

FIG. 84 is a graph of the flammability envelope constructed using data from FIG. 82 and following the methodology described in Example 24. The experimental data points (circles) are from tests described herein that were conducted at 1 atmosphere initial system pressure.

FIG. 85 is a graph of the flammability envelope constructed using data from FIG. 83 and following the methodology described in Example 24. The experimental data points (circles) are from tests described herein that were conducted at 1 atmosphere initial system pressure.

FIG. 86A is a GC/MS chromatogram of fermentation off-gas.

FIG. 86B is an expansion of FIG. 86A to show minor volatiles present in fermentation off-gas.

FIG. 87A is a GC/MS chromatogram of trace volatiles present in off-gas following cryo-trapping at −78° C.

FIG. 87B is a GC/MS chromatogram of trace volatiles present in off-gas following cryo-trapping at −196° C.

FIG. 87C is an expansion of FIG. 87B.

FIG. 87D is an expansion of FIG. 87C.

FIGS. 88A and 88B are GC/MS chromatogram comparing C5 hydrocarbons from petroleum-derived isoprene (FIG. 88A) and biologically produced isoprene (FIG. 88B). The standard contains three C5 hydrocarbon impurities eluting around the main isoprene peak (FIG. 88A). In contrast, biologically produced isoprene contains amounts of ethanol and acetone (run time of 3.41 minutes) (FIG. 88A).

FIG. 89 is a graph of the analysis of fermentation off-gas of an E. coli BL21 (DE3) pTrcIS strain expressing a Kudzu isoprene synthase and fed glucose with 3 g/L yeast extract.

FIG. 90 shows the structures of several impurities that are structurally similar to isoprene and may also act as polymerization catalyst poisons.

FIG. 91 is a map of pTrcHis2AUpperPathway (also called pTrcUpperMVA).

FIGS. 92A-92C are the nucleotide sequence of pTrcHis2AUpperPathway (also called pTrcUpperMVA) (SEQ ID NO:86).

FIG. 93 is a time course of optical density within the 15-L bioreactor fed with glucose.

FIG. 94 is a time course of isoprene titer within the 15-L bioreactor fed with glucose. The titer is defined as the amount of isoprene produced per liter of fermentation broth.

FIG. 95 is a time course of total isoprene produced from the 15-L bioreactor fed with glucose.

FIG. 96A is a map of MCM380-pTrcKudzuMVK (Lactobacillus sakei).

FIGS. 96B and 96C are the nucleotide sequence of MCM380-pTrcKudzuMVK (Lactobacillus sakei) (SEQ ID NO:107).

FIG. 97A is a map of MCM379-pTrcKudzuMVK (Streptococcus pneumoniae).

FIGS. 97B and 97C are the nucleotide sequence of MCM379-pTrcKudzuMVK (Streptococcus pneumoniae) (SEQ ID NO:108).

FIG. 98A is a map of MCM381-pTrcKudzuMVK (Streptomyces CL190).

FIGS. 98B and 98C are the nucleotide sequence of MCM381-pTrcKudzuMVK (Streptomyces CL190) (SEQ ID NO:109).

FIG. 99 is a time course of optical density within the 15-L bioreactor fed with glucose.

FIG. 100 is a time course of isoprene titer within the 15-L bioreactor fed with glucose. The titer is defined as the amount of isoprene produced per liter of fermentation broth.

FIG. 101 is a time course of isoprene specific activity from the 15-L bioreactor fed with glucose.

FIG. 102 is a map of pCLPtrcUpperPathwayHGS2 (also referred to as pCL UpperHGS2).

FIGS. 103A-103C are the nucleotide sequence of pCLPtrcUpperPathwayHGS2 (SEQ ID NO:87).

FIG. 104 is a time course of optical density within the 15-L bioreactor fed with glucose.

FIG. 105 is a time course of isoprene titer within the 15-L bioreactor fed with glucose. The titer is defined as the amount of isoprene produced per liter of fermentation broth.

FIG. 106 is a time course of total isoprene produced from the 15-L bioreactor fed with glucose.

FIG. 107 is a map of plasmid MCM330.

FIGS. 108A-108C are the nucleotide sequence of plasmid MCM330 (SEQ ID NO:90).

FIG. 109 is a map of pET24D-Kudzu.

FIGS. 110A and 110B are the nucleotide sequence of pET24D-Kudzu (SEQ ID NO:101).

FIG. 111A is a time course of optical density within the 15-L bioreactor fed with glucose.

FIG. 111B is a time course of isoprene titer within the 15-L bioreactor fed with glucose. The titer is defined as the amount of isoprene produced per liter of fermentation broth.

FIG. 111C is a time course of specific productivity of isoprene in the 15-L bioreactor fed with glucose.

FIG. 112A is a graph of the growth of MCM127 in TM3 media at 30° C. measured as optical density (OD600). One culture was induced with 150 μM IPTG 4 hours after inoculation.

FIG. 112B is a graph of the accumulated key metabolic intermediates after induction of MCM127 with 150 μM IPTG. The culture was induced 4 hours after inoculation and samples were analyzed using LCMS.

FIGS. 112C-112K are isoprene fermentation expressing genes from the MVA pathway and grown in fed-batch culture at the 15-L scale in different E. coli strains (MCM343 strain (FIGS. 112C-112E); MCM127 strain (FIGS. 112F-112H); dxr knock-out strain (FIGS. 112I-112K)). FIGS. 112C, 112F, and 112I show the time course of optical density within the 15-L bioreactor fed with glucose in MCM343 strain, MCM127 strain, and dxr knock-out strain, respectively. FIGS. 112D, 112G, and 112J are the time course of isoprene titer within the 15-L bioreactor fed with glucose in MCM343 strain, MCM127 strain, and dxr knock-out strain, respectively. The titer is defined as the amount of isoprene produced per liter of fermentation broth. FIGS. 112E, 112H, and 112K are the time course of total isoprene produced from the 15-L bioreactor fed with glucose in MCM343 strain, MCM127 strain, and dxr knock-out strain, respectively.

FIGS. 112L-112N depict the construction and phenotype of the dxr mutant in E. coli. 1-deoxy-D-xylulose 5-phosphate reductoisomerase (dxr) was deleted using the GeneBridges Quick & Easy E. coli Gene Deletion Kit. FIG. 112L shows the chromosomal location of dxr (from EcoCyc) and the approximate primer binding sites for testing the insertion of the GB resistance cassette. FIG. 112M is a PCR analysis of dxr deletion strains (in MG1655) using primers dxrTest1 and GBprimer2 (GB2), and dxrTest2 and GBprimerDW (GB3). PCR products were run on an Egel (Invitrogen) according to the manufacturer's protocol. FIG. 112N shows the inhibition of the growth of dxr deletion strains at 10 mM MVA. DW28 were grown overnight at 37° C. on LB medium plates containing spectinomycin 50 μg/ml, chloramphenicol 25 μg/ml, and the indicated concentrations of MVA.

FIG. 112O lists forward and reverse primers for pCL Ptrc(minus lacO) UpperPathway: forward primer MCM63 (SEQ ID NO:139) and reverse primer MCM64 (SEQ ID NO:140).

FIG. 112P is a map of MCM184-pCL Ptrc(minus lacO) UpperPathway.

FIG. 112Q-112S are the nucleotide sequence of MCM184 (SEQ ID NO:141).

FIG. 112T lists PCR and sequencing primers for pCL Ptrc (AlacO)KKDyI: primer EL-976 (SEQ ID NO:142), primer EL-977 (SEQ ID NO:143), and primer EL-978 (SEQ ID NO:144).

FIG. 112U is a map of pCL Ptrc (AlacO)KKDyI.

FIGS. 112V-112X are the nucleotide sequence of pCL Ptrc (AlacO)KKDyI (SEQ ID NO:145).

FIGS. 113A-113D demonstrate that over-expression of MVK and isoprene synthase results in increased isoprene production. Accumulated isoprene and CO₂ from MCM401 and MCM343 during growth on glucose in 100 mL bioreactors with 100 and 200 uM IPTG induction of isoprene production was measured over a 22 hour time course. FIG. 113A is a graph of the accumulated isoprene (%) from MCM343. FIG. 113B is a graph of the accumulated isoprene (%) from MCM401. FIG. 113C is a graph of the accumulated CO₂ (%) from MCM343. FIG. 113D is a graph of the accumulated CO₂ (%) from MCM401.

FIG. 114 is a time course of optical density within the 15-L bioreactor fed with glucose.

FIG. 115 is a time course of isoprene titer within the 15-L bioreactor fed with glucose. The titer is defined as the amount of isoprene produced per liter of fermentation broth.

FIG. 116 is a time course of total isoprene produced from the 15-L bioreactor fed with glucose.

FIG. 117 is a time course of optical density within the 15-L bioreactor fed with glucose.

FIG. 118 is a time course of isoprene titer within the 15-L bioreactor fed with glucose. The titer is defined as the amount of isoprene produced per liter of fermentation broth.

FIG. 119 is a time course of total isoprene produced from the 15-L bioreactor fed with glucose.

FIG. 120 is a time course of optical density within the 15-L bioreactor fed with glucose.

FIG. 121 is a time course of isoprene titer within the 15-L bioreactor fed with glucose. The titer is defined as the amount of isoprene produced per liter of fermentation broth.

FIG. 122 is a time course of total isoprene produced from the 15-L bioreactor fed with glucose.

FIG. 123 is a time course of optical density within the 15-L bioreactor fed with glucose.

FIG. 124 is a time course of isoprene titer within the 15-L bioreactor fed with glucose. The titer is defined as the amount of isoprene produced per liter of fermentation broth.

FIG. 125 is a time course of total isoprene produced from the 15-L bioreactor fed with glucose.

FIGS. 126A and 126B are the nucleotide sequence of pDU-5 MVK from S. cerevsiae in pET-16b (SEQ ID NO:111).

FIG. 127A is a map of pDW01.

FIGS. 127B and 127C are the nucleotide sequence of pDW01 (ORF of 6XHis-Lb. sakei Mvk is underlined) (SEQ ID NO:112).

FIG. 128A is a map of pDW02.

FIGS. 128B and 128C are the nucleotide sequence of pDWO2 (ORF of 6XHis-S. pneumoniae Mvk is underlined) (SEQ ID NO:113).

FIG. 129 is a picture of a gel showing the induction of Lb. sakei and S. pneumoniae MVK constructs. This gel shows expression of Lactobacillus sakei and Streptococcus pneumoniae MVK in BL21 Star (DE3) (Invitrogen). Cells were grown to late exponential phase (OD600˜1) and induced with 1 mM IPTG. After 2 hours of induction (at 37° C.) samples were removed and visualized on a 4-12% Novex SDS gel (Nupage—Invitrogen). The SeeBlue Plus2 standard (Invitrogen) was used to visualize approximate molecular weights. Lane 1˜Lb. sakei Mvk (pDW01) and no IPTG; lane 2—pDW01 and 1 mM IPTG; lane 3—S. pneumoniae Mvk (pDW02) and no IPTG; lane 4—pDW02 and 1 mM IPTG; lane 5—S. pneumoniae Mvk (pDW02 isolate #2) and no IPTG; lane 6—pDW02 (isolate #2) and 1 mM IPTG. The arow on the left indicates the induced band from pDW01; the arrow on the right indicates the induced bands from pDW02 and pDW02#2 in lanes 4 and 6.

FIG. 130 is a time course of optical density within the 15-L bioreactor fed with glucose.

FIG. 131 is a time course of isoprene titer within the 15-L bioreactor fed with glucose. The titer is defined as the amount of isoprene produced per liter of fermentation broth.

FIG. 132 is a time course of total isoprene produced from the 15-L bioreactor fed with glucose.

FIG. 133 is a time course of volumetric productivity within the 15-L bioreactor fed with glucose. The volumetric productivity is defined as the amount of isoprene produced per liter of broth per hour.

FIG. 134 is a time course of instantaneous yield within the 15-L bioreactor fed with glucose. The instantaneous yield is defined as the amount of isoprene (gram) produced per amount of glucose (gram) fed to the bioreactor (w/w) during the time interval between the data points.

FIG. 135 is a time course of optical density within the 15-L bioreactor fed with glucose.

FIG. 136 is a time course of isoprene titer within the 15-L bioreactor fed with glucose. The titer is defined as the amount of isoprene produced per liter of fermentation broth.

FIG. 137 is a time course of total isoprene produced from the 15-L bioreactor fed with glucose.

FIG. 138A is a map of plasmid MCM94-pTrcHis2B kan.

FIGS. 138B and 138C are the nucleotide sequence of plasmid MCM94-pTrcHis2B kan (SEQ ID NO:114).

FIG. 139 is a graph showing that over-expression of both isoprene synthase and MVK results in an increased specific productivity of isoprene compared to over-expression of each of the enzymes alone, or low expression of both enzymes. The specific productivity of isoprene using MCM343, MCM401, MCM437, and MCM438 during growth on glucose in mini-fermentations with 200 μM IPTG induction was measured over time. Error bars represent one standard deviation.

FIG. 140 is a typical elution profile of phosphorylated intermediates in the isoprenoid pathway extracted from the MCM391 strain of E. coli after 50 hours of fermentation and detected using LC-ESI-MS/MS.

FIGS. 141A-141F are graphs showing the accumulation of isoprenoid pathway intermediates in MCM401 strain of E. coli containing MVK from M. mazei upon different levels of enzyme expression. FIGS. 141A-141C show ODs and specific isoprene production of the cultures grown in 14-L fermentors, and FIGS. 141D-141F show intracellular levels of isoprenoid metabolites. Arrows on top of the figures indicate the time points when IPTG was added to fermentors (1−4×50 μM; 2−2×100 μM and 3−1×200 μM).

FIGS. 142A and 142B are graphs showing the accumulation of isoprenoid pathway intermediates in the MCM402 strain of E. coli containing MVK from yeast and grown in 14-L fermentors. Arrows on the top figure indicate the time points when 50 μM IPTG doses were added to fermentors.

FIGS. 143A and 143B are graphs showing the accumulation of isoprenoid pathway intermediates in the MCM400 strain of E. coli containing MVK from Streptomyces and grown in 14-L fermentor. Arrows on the top figure indicate the time points when 50 μM IPTG doses were added to the fermentor.

FIGS. 144A and 144B are graphs showing the accumulation of isoprenoid pathway intermediates in the MCM343 strain of E. coli. Arrows on the top figure indicate the time point when 100 μM IPTG dose was added to the fermentor.

FIG. 145 is a graph of growth curves for cultures of BL21 expressing MVK, circles; MVK+PMV, triangles; MVK+PMV+MDD, squares. Cultures were either fed 5.8 mM MVA, filled symbols, or grown without addition of MVA, open symbols. Y-axis is OD₆₀₀. Samples were taken for analysis at times indicated by the arrow. Numbers above the arrows correspond to E. coli BL21 cells bearing pTrcK, representing a plasmid expressing MVK (#5), pTrcKK representing a plasmid expressing MVK plus PMK (#7), and pTrcKKD, representing a plasmid expressing MVK plus PMK plus MDD (#6) were grown.

FIG. 146 is a graph of isoprene synthase (IS) activity versus volumetric productivity in strains MCM127, MCM343, and MCM401.

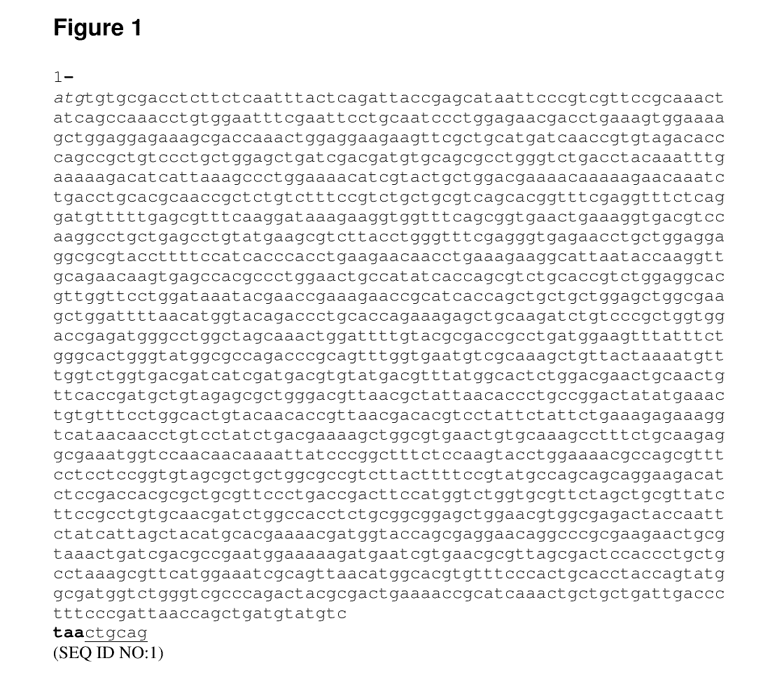

### DETAILED DESCRIPTION OF THE INVENTION

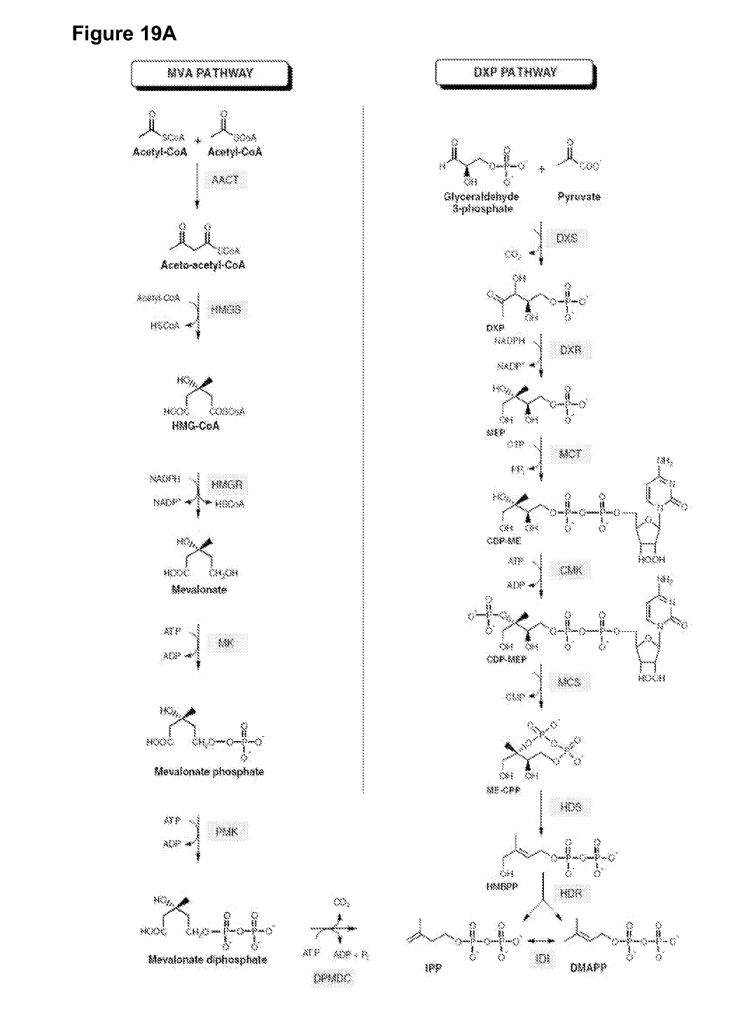

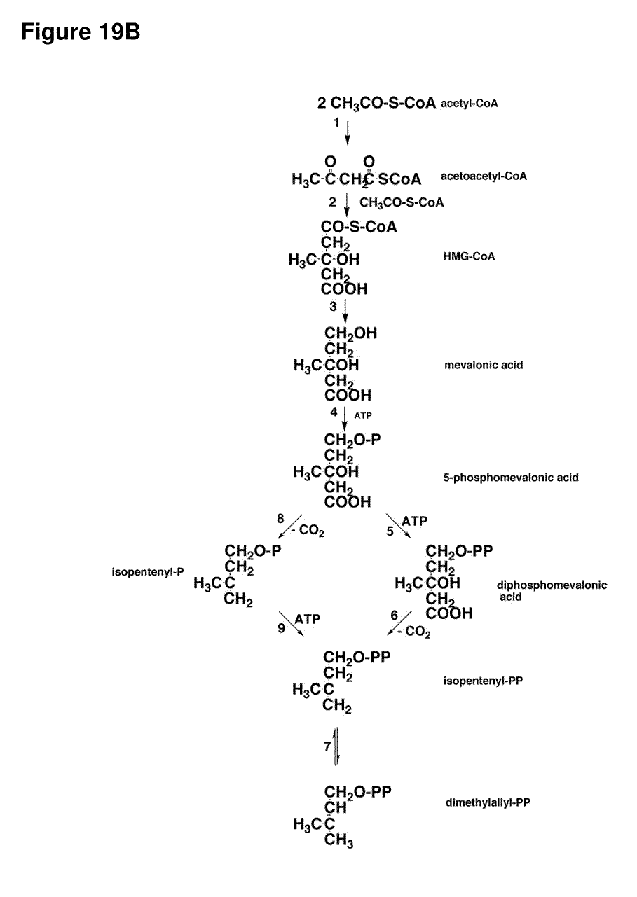

As illustrated in FIGS. 19A and 19B, mevalonate kinase (MVK) polypeptides phosphorylate mevalonate (MVA) to form mevalonate-5-phosphate (MVAP), as part of the MVA pathway for the biosynthesis of isoprene. Isoprene synthase polypeptides convert dimethylallyl diphosphate (DMAPP) into isoprene. As used herein, the term “isoprene” or “2-methyl-1,3-butadiene” (CAS#78-79-5) refers to the direct and final volatile C5 hydrocarbon product from the elimination of pyrophosphate from 3,3-dimethylallyl pyrophosphate (DMAPP), and does not involve the linking or polymerization of one or more isopentenyl diphosphate (IPP) molecules to one or more DMAPP molecules.

Both a high flux from central metabolism to DMAPP and a robust enzyme activity to catalyze the conversion of DMAPP to isoprene are desirable for the commercial scale production of isoprene in vivo. Increasing MVK polypeptide activity is desirable because it reduces the accumulation of MVA and increases the supply of MVAP for conversion to isoprene using the MVA pathway. Since high concentrations of DMAPP are growth inhibitory, high flux through the MVA pathway is desirably accompanied by high isoprene synthase polypeptide activity to avoid accumulation of toxic amounts of DMAPP. Accordingly, in one aspect, the invention features a method of producing isoprene that involves increasing the expression and/or activity of (i) a MVK polypeptide and (ii) an isoprene synthase polypeptide compared to the expression level and/or activity level normally found in the cell. For example, overexpressing the MVK polypeptide from M. mazei and the isoprene synthase from kudzu supports high flux to DMAPP and simultaneous conversion of DMAPP to isoprene. Furthermore, by balancing the activity of the MVK polypeptide and the isoprene synthase polypeptide, we have generated cells which convert acetyl-CoA to isoprene at high flux and titer without the accumulation of DMAPP. The total activity level of an MVK polypeptide is influenced by both the level of protein expressed and the enzymatic characteristics of the specific MVK polypeptide used. Limiting the accumulation of DMAPP is valuable because it prevents DMAPP-associated growth inhibition and loss of metabolic activity.

As described further in the Examples, overexpression of the M. mazei MVK polypeptide and the kudzu isoprene synthase polypeptide resulted in an eight-fold increase in isoprene titer compared to overexpression of isoprene synthase alone. As discussed in Examples 3-5, E. coli cells containing the MVA pathway (pCL PtrcUpperPathway encoding E. faecalis mvaE and mvaS), the integrated lower MVA pathway (gi1.2KKDyI encoding S. cerevisiae mevalonate kinase, mevalonate phosphate kinase, mevalonate pyrophosphate decarboxylase, and IPP isomerase), and high expression of mevalonate kinase from M. mazei and isoprene synthase from kudzu (pTrcKudzuMVK(M. mazei)) were used to produce isoprene in 15-L bioreactors. Example 3 indicates that the total amount of isoprene produced during a 68 hour fermentation was 227.2 g. Instantaneous volumetric productivity levels reached values as high as 1.5 g isoprene/L broth/hr, and the instantaneous yield levels reached as high as 17.7% w/w (Example 4). Example 5 indicates that the molar yield of utilized carbon that went into producing isoprene during this fermentation was 16.6%, and the weight percent yield of isoprene from glucose over the entire fermentation was 7.7%. Additionally, overexpression of a kudzu isoprene synthase polypeptide and either a Streptomyces MVK polypeptide (Example 9), Lactobacillus MVK polypeptide (Example 10), or Saccharomyces MVK polypeptide (Example 11) also resulted in the production of significant amounts of isoprene. Additionally, Example 12 describes the expression of Lactobacillus sakei and Streptococcus pneumoniae mevalonate kinase polypeptides. These Examples support the general applicability of overexpres sing both an MVK polypeptide and an isoprene synthase polypeptide to increase production of isoprene.

Example 6 describes the comparison of four strains with different relative levels of isoprene synthase polypeptide activity and MVK polypeptide activity: (i) the MCM343 strain with low MVK polypeptide activity and high isoprene synthase polypeptide activity, (ii) the MCM401 strain with high MVK polypeptide activity and high isoprene synthase polypeptide activity, (iii) the MCM437 with low MVK polypeptide activity and low isoprene synthase, and (iv) the MCM438 strain with high MVK polypeptide activity and low isoprene synthase polypeptide activity. In particular, the specific productivity of isoprene from a strain expressing the full mevalonic acid pathway and kudzu isoprene synthase polypeptide at low levels (MCM437) was compared to a strain that in addition over-expressed MVK polypeptide from M. mazei and kudzu isoprene synthase polypeptide (MCM401), as well as strains that either over-expressed just MVK polypeptide (MCM438), or just kudzu isoprene synthase polypeptide (MCM343). The strain over-expressing both MVK polypeptide and isoprene synthase polypeptide (MCM401) had higher specific productivity of isoprene compared to the strain over-expressing just MVK polypeptide (MCM438) or just kudzu isoprene synthase polypeptide (MCM343). The strain with low activities of both MVK polypeptide and kudzu isoprene synthase polypeptide (MCM437) had the lowest specific productivity of isoprene overall.

Accordingly, in some embodiments, the cells overexpress both an MVK polypeptide and an isoprene synthase polypeptide. In the experiments described in Examples 2-5, E. coli cells containing the upper mevalonic acid (MVA) pathway (pCL PtrcUpperPathway encoding E. faecalis mvaE and mvaS), the integrated lower MVA pathway (gi1.2KKDyI encoding S. cerevisiae mevalonate kinase, mevalonate phosphate kinase, mevalonate pyrophosphate decarboxylase, and IPP isomerase), and high expression of mevalonate kinase from M. mazei and isoprene synthase from kudzu (pTrcKudzuMVK(M. mazei) were used to produce isoprene. In these experiments, the M. mazei MVK polypeptide and kudzu isoprene synthase polypeptide were overexpressed from a high copy plasmid under the control of a strong promoter. In contrast, the S. cerevisiae lower MVA pathway nucleic acids (mevalonate kinase, mevalonate phosphate kinase, mevalonate pyrophosphate decarboxylase, and IPP isomerase) were present as a single copy of the nucleic acids integrated in the chromosome under the control of a weak promoter. The E. faecalis upper MVA pathway nucleic acids (mvaE encoding a naturally occurring fusion protein that has both acetyl-CoA acetyltransferase and 3-hydroxy-3-methylglutaryl-CoA reductase activities and mvaS encoding a 3-hydroxy-3-methylglutaryl-CoA synthase polypeptide) were overexpressed from a medium copy plasmid under the control of a strong promoter (the same promoter used to express the M. mazei MVK polypeptide and kudzu isoprene synthase polypeptide). Thus, the M. mazei MVK polypeptide and kudzu isoprene synthase polypeptide were expressed at a much higher level than the other MVA pathway polypeptides. Since the M. mazei MVK polypeptide was expressed at a much higher level than the S. cerevisiae MVK polypeptide, most of the conversion of MVA to MVAP seems to be due to the M. mazei MVK polypeptide rather than the S. cerevisiae MVK polypeptide. If desired, the S. cerevisiae MVK nucleic acid can be removed from any of the cells disclosed herein using standard methods (such that the only heterologous MVK nucleic acid is the M. mazei MVK nucleic acid). If desired, the S. cerevisiae MVK nucleic acid can alternatively be replaced by any other MVK nucleic acid in any of the cells described herein.

Accordingly, in some embodiments, an MVK polypeptide and/or an isoprene synthase polypeptide is expressed a level that is at least about any of 2, 5, 10, 20, 30, 40, 50, 60, 70, 80, 90, 100, 125, 150, 200, 225, 250, 275, 300, 350, 400, 450, or 500-fold (i) higher than the level of expression of a second MVA pathway polypeptide (such as an acetyl-CoA acetyltransferase (AACT) polypeptide, 3-hydroxy-3-methylglutaryl-CoA synthase (HMGS) polypeptide, 3-hydroxy-3-methylglutaryl-CoA reductase (HMGR) polypeptide, phosphomevalonate kinase (PMK) polypeptide, diphosphomevalonate decarboxylase (DPMDC) polypeptide, or isopentenyl-diphosphate delta-isomerase (IDI) polypeptide) or (ii) higher than the level of expression of all other MVA pathway polypeptides in the cell. In particular embodiments, the MVK polypeptide and/or an isoprene synthase polypeptide is expressed a level that is at least about any of 2, 5, 10, 12, 14, 16, 18, 20, 30, 40, 50, 60, 70, 80, 90, 100, 125, 150, 200, 225, 250, 275, 300, 350, 400, 450, or 500-fold higher than the level of expression of an AACT polypeptide, HMGS polypeptide, and HMGR polypeptide. In particular embodiments, the MVK polypeptide and/or an isoprene synthase polypeptide is expressed a level that is at least about any of 2, 5, 10, 20, 30, 40, 50, 60, 70, 80, 90, 100, 125, 150, 200, 225, 250, 275, 300, 350, 400, 450, or 500-fold higher than the level of expression of an PMK polypeptide, DPMDC polypeptide, and IDI polypeptide. In some embodiments, the total amount of MVK polypeptide is similar to the total amount of isoprene synthase polypeptide. For example, in some embodiments, the total amount of MVK polypeptide is within about any of 10, 8, 6, 4, 2, 1, or 0.5-fold higher or lower than the total amount of isoprene synthase polypeptide (e.g., the amount of MVK polypeptide may be between about 10-fold lower to about 10-fold higher than the amount of isoprene synthase polypeptide). Standard methods (such as western blotting) can be used to quantitate the amount of any of these polypeptides. Standard methods can be used to alter the relative amounts of expressed MVA pathway polypeptides, such as by using a stronger promoter or a plasmid with a higher copy number to express an MVK polypeptide and/or an isoprene synthase polypeptide compared to the promoter(s) and plasmid(s) used to express other MVA pathway polypeptides.

In some embodiments, an MVK RNA molecule and/or an isoprene synthase RNA molecule is expressed a level that is at least about any of 2, 5, 10, 20, 30, 40, 50, 60, 70, 80, 90, 100, 125, 150, 200, 225, 250, 275, 300, 350, 400, 450, or 500-fold (i) higher than the level of expression of a second MVA pathway RNA molecule (such as an AACT RNA molecule, HMGS RNA molecule, HMGR RNA molecule, PMK RNA molecule, DPMDC RNA molecule, or IDI RNA molecule) or (ii) higher than the level of expression of all other MVA pathway RNA molecules in the cell. In particular embodiments, the MVK RNA molecule and/or an isoprene synthase RNA molecule is expressed a level that is at least about any of 2, 5, 10, 12, 14, 16, 18, 20, 30, 40, 50, 60, 70, 80, 90, 100, 125, 150, 200, 225, 250, 275, 300, 350, 400, 450, or 500-fold higher than the level of expression of an AACT RNA molecule, HMGS RNA molecule, and HMGR RNA molecule. In particular embodiments, the MVK RNA molecule and/or an isoprene synthase RNA molecule is expressed a level that is at least about any of 2, 5, 10, 20, 30, 40, 50, 60, 70, 80, 90, 100, 125, 150, 200, 225, 250, 275, 300, 350, 400, 450, or 500-fold higher than the level of expression of an PMK RNA molecule, DPMDC RNA molecule, and IDI RNA molecule. In some embodiments, the total amount of MVK RNA is similar to the total amount of isoprene synthase RNA. For example, in some embodiments, the total amount of MVK RNA is within about any of 10, 8, 6, 4, 2, 1, or 0.5-fold higher or lower than the total amount of isoprene synthase RNA (e.g., the amount of MVK RNA may be between about 10-fold lower to about 10-fold higher than the amount of isoprene synthase RNA). Standard methods (such as northern blotting) can be used to quantitate the amount of any of these RNA molecules. Standard methods can be used to alter the relative amounts of expressed MVA pathway RNA molecules, such as by using a stronger promoter or a plasmid with a higher copy number to express an MVK RNA molecule and/or an isoprene synthase RNA molecule compared to the promoter(s) and plasmid(s) used to express other MVA pathway RNA molecules.

In some embodiments, the number of copies of an MVK DNA molecule and/or an isoprene synthase DNA molecule is at least about any of 2, 5, 10, 20, 30, 40, 50, 60, 70, 80, 90, 100, 125, 150, 200, 225, 250, 275, 300, 350, 400, 450, or 500-fold (i) higher than the number of copies of a second MVA pathway DNA molecule (such as an AACT DNA molecule, HMGS DNA molecule, HMGR DNA molecule, PMK DNA molecule, DPMDC DNA molecule, or IDI DNA molecule) or (ii) higher than the number of copies of all other MVA pathway DNA molecules in the cell. In particular embodiments, the number of copies of an MVK DNA molecule and/or an isoprene synthase DNA molecule is at least about any of 2, 5, 10, 12, 14, 16, 18, 20, 30, 40, 50, 60, 70, 80, 90, 100, 125, 150, 200, 225, 250, 275, 300, 350, 400, 450, or 500-fold higher than the number of copies of an AACT DNA molecule, HMGS DNA molecule, and HMGR DNA molecule. In particular embodiments, the number of copies of a MVK DNA molecule and/or an isoprene synthase DNA molecule is at least about any of 2, 5, 10, 20, 30, 40, 50, 60, 70, 80, 90, 100, 125, 150, 200, 225, 250, 275, 300, 350, 400, 450, or 500-fold higher than the number of copies of an PMK DNA molecule, DPMDC DNA molecule, and IDI DNA molecule. In some embodiments, the number of copies of an MVK DNA molecule is similar to the number of copies of an isoprene synthase DNA molecule. For example, in some embodiments, the number of copies of an MVK DNA molecule is within about any of 10, 8, 6, 4, 2, 1, or 0.5-fold higher or lower than the number of copies of an isoprene synthase DNA molecule (e.g., the number of copies of a MVK DNA may be between about 10-fold lower to about 10-fold higher than the number of copies of an isoprene synthase DNA molecule). Standard methods (such as southern blotting) can be used to quantitate the amount of any of these DNA molecules. Standard methods can be used to alter the relative amounts of MVA pathway DNA molecules, such as by using a plasmid with a higher copy number to insert an MVK DNA molecule and/or an isoprene synthase DNA molecule compared to the plasmid(s) used to insert other MVA pathway DNA molecules.

As discussed above, increasing the expression of an MVK polypeptide, decreases that amount of MVA that accumulates in the cell medium since more MVA is converted to MVAP. Increasing the expression of an isoprene synthase polypeptide decreases the accumulation of DMAPP since more DMAPP is converted to isoprene. If desired, the expression of a PMK polypeptide, DPMDC polypeptide, IDI polypeptide, or any combination of two or more of the foregoing can also be increased to reduce the accumulation of MVA pathway or isoprenoid biosynthesis intermediates and/or to increase the flux through the MVA pathway. In some embodiments, the amount of mevalonate (MVA), 3,3-dimethylallyl diphosphate (DMAPP), isopentenyl diphosphate (IPP), geranyl diphosphate (GPP), farnesyl diphosphate (FPP), or any combination of two or more of the foregoing allows production of isoprene without causing undesirable amounts of growth inhibition, toxicity, or cell death. In some embodiments, the amount of MVA, DMAPP, and/or IPP is high enough to allow production of isoprene in any of the amounts or concentrations disclosed below in the “Exemplary Production of Isoprene” section. In some embodiments, a detectable amount of MVA, DMAPP, and/or IPP does not accumulate since the intermediate(s) are being converted to downstream molecules at a rate that does not allow a detectable amount of MVA, DMAPP, and/or IPP to accumulate. Example 8, parts IV, V, and VI indicate that overexpression of either the M. mazei MVK polypeptide or the Streptomyces MVK polypeptide is correlated with the accumulation of less DMAPP and IPP than overexpression of the S. cerevisiae MVK polypeptide. A goal is therefore to achieve a pathway enzyme balance to minimize the accumulation of these metabolites for the relief of growth inhibition.

Tables 15A and 15B list exemplary desirable concentrations of DMAPP, IPP, GPP, and FPP as well as examples of relatively high concentrations of these metabolites that have been detected using the cells and methods described herein. Table 15B has the same data as Table 15A that has been normalized to grams of dry cell weight assuming that 1 liter of the culture at OD=1 has 0.33 grams dry cell weight (g_{dcw}). For these experiments, the quantitation limit is below 0.1 mM for the intracellular concentrations of DMAPP, FPP, GPP, and IPP. In desired, more sensitive equipment can be used to detect even smaller amounts of these compounds. The lowest absolute concentrations that were used as standards for the LCMS calibration of these compounds was 3.4 uM DMAPP, 1.7 uM IPP, 0.9 uM GPP, and 2.3 uM FPP. Thus, absolute amounts that are equal to or greater than these standard amounts can be readily detected.

In these experiments, there was a negligible amount of DMAPP, FPP, GPP, and IPP in the liquid cell medium (outside of the cells). Thus, the amounts listed in Tables 15A and 15B are representative of the intracellular concentrations of DMAPP, FPP, GPP, and IPP.

TABLE 15A
Exemplary metabolite concentrations

| | | Metabolite | | | |
|---|---|---|---|---|---|
| | | DMAPP | IPP | GPP | FPP |
| Intracellular | Exemplary | 0.4 mM ¹ | 0.3 mM ¹ | 0.7 mM ² | 1.4 mM ¹ |
| concentration, | desirable | | | | |
| mM | concentrations | | | | |
| | Exemplary | 9.2 mM ³ | 27-40 mM ⁴ | 2.8 mM ³ | 3.6 mM ³ |
| | detected | 15.3 mM ⁵ | 6.3 mM ⁵ | 3.3 mM ⁵ | |
| | concentrations | | | | |

¹ Example 3.
² Example 8, Part VII.
³ Example 7, Part III.
⁴ Example 8, Part VIII.
⁵ Example 7, Part II.

TABLE 15B
Exemplary metabolite concentrations

| | | Metabolite | | | |
|---|---|---|---|---|---|
| | | DMAPP | IPP | GPP | FPP |
| Intracellular | Exemplary | 0.3 ¹ | 0.2 ¹ | 0.5 ² | 1.1 ¹ |
| concentration, | desirable | | | | |
| μmol/g_{dcw}⁶ | concentrations | | | | |
| | Exemplary | 7.0 ³ | 20-30 ⁴ | 2.1 ³ | 2.0 ³ |
| | detected | 11.6 ⁵ | 4.8 ⁵ | 3.3 ⁵ | |
| | concentrations | | | | |

¹ Example 3.
² Example 8, Part VII.
³ Example 7, Part III.
⁴ Example 8, Part VIII.
⁵ Example 7, Part II.

In some embodiments, the intracellular concentration of DMAPP is between about 0 to about 25 μmol/g_{dcw}, such as between about 0.1 to about 20 μmol/g_{dcw}, about 0.1 to about 15 μmol/g_{dcw}, about 0.1 to about 11 μmol/g_{dcw}, about 0.1 to about 7 μmol/g_{dcw}, about 0.1 to about 5 μmol/g_{dcw}, about 0.1 to about 2 μmol/g_{dcw}, about 0.1 to about 1 μmol/g_{dcw}, about 0.1 to about 0.8 μmol/g_{dcw}, about 0.1 to about 0.6 μmol/g_{dcw}, about 0.2 to about 15 μmol/g_{dcw}, about 0.2 to about 11 μmol/g_{dcw}, about 0.2 to about 7 μmol/g_{dcw}, about 0.2 to about 5 μmol/g_{dcw}, about 0.2 to about 2 μmol/g_{dcw}, about 0.3 to about 11 μmol/g_{dcw}, about 0.3 to about 7 μmol/g_{dcw}, about 0.3 to about 5 μmol/g_{dcw}, about 0.3 to about 2 μmol/g_{dcw}, about 0.3 to about 1 μmol/g_{dcw}, about 0.4 to about 11 μmol/g_{dcw}, about 0.4 to about 7 μmol/g_{dcw}, about 0.4 to about 5 μmol/g_{dcw}, about 0.4 to about 2 μmol/g_{dcw}, about 0.5 to about 7 μmol/g_{dcw}, about 0.5 to about 5 μmol/g_{dcw}, or about 0.5 to about 2 μmol/g_{dcw}. In some embodiments, the intracellular concentration of DMAPP is equal to or less than about any of 25, 20, 18, 16, 14, 12, 10, 8, 6, 4, 2, 1, 0.8, 0.6, 0.5, 0.4, 0.3, 0.2, or 0.1 μmol/g_{dcw}.

In some embodiments, the intracellular concentration of IPP is between about 0 to about 60 μmol/g_{dcw}, such as between about 0.1 to about 50 μmol/g_{dcw}, about 0.1 to about 40 μmol/g_{dcw}, about 0.1 to about 30 μmol/g_{dcw}, about 0.1 to about 20 μmol/g_{dcw}, about 0.1 to about 15 μmol/g_{dcw}, about 0.1 to about 11 μmol/g_{dcw}, about 0.1 to about 7 μmol/g_{dcw}, about 0.1 to about 5 μmol/g_{dcw}, about 0.1 to about 2 μmol/g_{dcw}, about 0.1 to about 1 μmol/g_{dcw}, about 0.1 to about 0.8 μmol/g_{dcw}, about 0.1 to about 0.6 μmol/g_{dcw}, about 0.2 to about 60 μmol/g_{dcw}, about 0.2 to about 50 μmol/g_{dcw}, about 0.2 to about 40 μmol/g_{dcw}, about 0.2 to about 30 μmol/g_{dcw}, about 0.2 to about 20 μmol/g_{dcw}, about 0.2 to about 15 μmol/g_{dcw}, about 0.2 to about 11 μmol/g_{dcw}, about 0.2 to about 7 μmol/g_{dcw}, about 0.2 to about 5 μmol/g_{dcw}, about 0.2 to about 2 μmol/g_{dcw}, about 0.3 to about 60 μmol/g_{dcw}, about 0.3 to about 50 μmol/g_{dcw}, about 0.3 to about 40 μmol/g_{dcw}, about 0.3 to about 30 μmol/g_{dcw}, about 0.3 to about 15 μmol/g_{dcw}, about 0.3 to about 11 μmol/g_{dcw}, about 0.3 to about 7 μmol/g_{dcw}, about 0.3 to about 5 μmol/g_{dcw}, about 0.3 to about 2 μmol/g_{dcw}, about 0.4 to about 60 μmol/g_{dcw}, about 0.4 to about 50 μmol/g_{dcw}, about 0.4 to about 40 μmol/g_{dcw}, about 0.4 to about 30 μmol/g_{dcw}, about 0.4 to about 15 μmol/g_{dcw}, about 0.4 to about 7 μmol/g_{dcw}, about 0.4 to about 5 μmol/g_{dcw}, about 0.4 to about 2 μmol/g_{dcw}, about 0.5 to about 60 μmol/g_{dcw}, about 0.5 to about 50 μmol/g_{dcw}, about 0.5 to about 40 μmol/g_{dcw}, about 0.5 to about 30 μmol/g_{dcw}, about 0.5 to about 15 μmol/g_{dcw}, about 0.5 to about 11 μmol/g_{dcw}, about 0.5 to about 7 μmol/g_{dcw}, about 0.5 to about 5 μmol/g_{dcw}, or about 0.5 to about 2 μmol/g_{dcw}. In some embodiments, the intracellular concentration of IPP is equal to or less than about any of 60, 50, 40, 30, 25, 20, 18, 16, 14, 12, 10, 8, 6, 4, 2, 1, 0.8, 0.6, 0.5, 0.4, 0.3, 0.2, or 0.1 μmol/g_{dcw}.

In some embodiments, the intracellular concentration of GPP is between about 0 to about 8 μmol/g_{dcw}, such as between about 0.1 to about 7 μmol/g_{dcw}, about 0.1 to about 6 μmol /g_{dcw}, about 0.1 to about 5 μmol/g_{dcw}, about 0.1 to about 4 μmol/g_{dcw}, about 0.1 to about 3 μmol/g_{dcw}, about 0.1 to about 2 μmol/g_{dcw}, about 0.1 to about 1 μmol/g_{dcw}, about 0.1 to about 0.8 μmol/g_{dcw}, about 0.1 to about 0.6 μmol/g_{dcw}, about 0.2 to about 7 μmol/g_{dcw}, about 0.2 to about 6 μmol/g_{dcw}, about 0.2 to about 5 μmol/g_{dcw}, about 0.2 to about 4 μmol/g_{dcw}, about 0.2 to about 3 μmol/g_{dcw}, about 0.2 to about 2 μmol/g_{dcw}, about 0.3 to about 7 μmol/g_{dcw}, about 0.3 to about 6 μmol/g_{dcw}, about 0.3 to about 5 μmol/g_{dcw}, about 0.3 to about 4 μmol/g_{dcw}, about 0.3 to about 3 μmol/g_{dcw}, about 0.3 to about 2 μmol/g_{dcw}, about 0.4 to about 7 μmol/g_{dcw}, about 0.4 to about 6 μmol/g_{dcw}, about 0.4 to about 5 μmol/g_{dcw}, about 0.4 to about 2 μmol/g_{dcw}, about 0.5 to about 7 μmol/g_{dcw}, about 0.5 to about 5 μmol/g_{dcw}, about 0.5 to about 2 μmol/g_{dcw}, about 0.6 to about 7 μmol/g_{dcw}, about 0.6 to about 5 μmol/g_{dcw}, about 0.6 to about 2 μmol/g_{dcw}, about 0.7 to about 7 μmol/g_{dcw}, about 0.7 to about 5 μmol/g_{dcw}, or about 0.7 to about 2 μmol/g_{dcw}. In some embodiments, the intracellular concentration of GPP is equal to or less than about any of 8, 6, 4, 2, 1, 0.8, 0.6, 0.5, 0.4, 0.3, 0.2, or 0.1 μmol/g_{dcw}.

In some embodiments, the intracellular concentration of FPP is between about 0 to about 6 μmol/g_{dcw}, such as between about 0.1 to about 6 μmol/g_{dcw}, about 0.1 to about 5 μmol/g_{dcw}, about 0.1 to about 4 μmol/g_{dcw}, about 0.1 to about 3 μmol/g_{dcw}, about 0.1 to about 2 μmol/g_{dcw}, about 0.1 to about 1 μmol/g_{dcw}, about 0.1 to about 0.8 μmol/g_{dcw}, about 0.1 to about 0.6 μmol/g_{dcw}, about 0.2 to about 6 μmol/g_{dcw}, about 0.2 to about 5 μmol/g_{dcw}, about 0.2 to about 4 μmol/g_{dcw}, about 0.2 to about 3 μmol/g_{dcw}, about 0.2 to about 2 μmol/g_{dcw}, about 0.3 to about 6 μmol/g_{dcw}, about 0.3 to about 5 μmol/g_{dcw}, about 0.3 to about 4 μmol/g_{dcw}, about 0.3 to about 3 μmol/g_{dcw}, about 0.3 to about 2 μmol/g_{dcw}, about 0.4 to about 6 μmol/g_{dcw}, about 0.4 to about 5 μmol/g_{dcw}, about 0.4 to about 2 μmol/g_{dcw}, about 0.5 to about 6 μmol/g_{dcw}, about 0.5 to about 5 μmol/g_{dcw}, about 0.5 to about 2 μmol/g_{dcw}, about 0.8 to about 6 μmol/g_{dcw}, about 0.8 to about 5 μmol/g_{dcw}, about 0.8 to about 2 μmol/g_{dcw}, about 1 to about 6 μmol/g_{dcw}, about 1 to about 5 μmol/g_{dcw}, about 1 to about 2 μmol/g_{dcw}, about 1.1 to about 6 μmol/g_{dcw}, about 1.1 to about 5 μmol/g_{dcw}, about 1.1 to about 2 μmol/g_{dcw}, about 1.1 to about 1.5 μmol/g_{dcw}, about 1.2 to about 6 μmol/g_{dcw}, about 1.2 to about 5 μmol/g_{dcw}, about 1.2 to about 2 μmol/g_{dcw}, or about 1.2 to about 1.5 μmol/g_{dcw}. In some embodiments, the intracellular concentration of FPP is equal to or less than about any of 6, 4, 2, 1.5, 1.4, 1.3, 1.2, 1.1, 1, 0.9, 0.8, 0.6, 0.5, 0.4, 0.3, 0.2, or 0.1 μmol/g_{dcw}.

In some embodiments, the concentration (e.g., concentration in the cell medium) of MVA is between about 0 to about 120 g/L, such as between about about 0 to about 110 g/L, such as between about 0.1 to about 100 g/L, about 0.1 to about 75 g/L, about 0.1 to about 60 g/L, about 0.1 to about 50 g/L, about 0.1 to about 40 g/L, about 0.1 to about 30 g/L, about 0.1 to about 20 g/L, about 0.1 to about 15 g/L, about 0.1 to about 11 g/L, about 0.1 to about 7 g/L, about 0.1 to about 5 g/L, about 0.1 to about 2 g/L, about 0.1 to about 1 g/L, about 0.1 to about 0.8 g/L, about 0.1 to about 0.6 g/L, about 0.2 to about 120 g/L, about 0.2 to about 100 g/L, about 0.2 to about 75 g/L, about 0.2 to about 60 g/L, about 0.2 to about 50 g/L, about 0.2 to about 40 g/L, about 0.2 to about 30 g/L, about 0.2 to about 20 g/L, about 0.2 to about 15 g/L, about 0.2 to about 11 g/L, about 0.2 to about 7 g/L, about 0.2 to about 5 g/L, about 0.2 to about 2 g/L, about 0.3 to about 120 g/L, about 0.3 to about 100 g/L, about 0.3 to about 75 g/L, about 0.3 to about 60 g/L, about 0.3 to about 50 g/L, about 0.3 to about 40 g/L, about 0.3 to about 30 g/L, about 0.3 to about 15 g/L, about 0.3 to about 11 g/L, about 0.3 to about 7 g/L, about 0.3 to about 5 g/L, about 0.3 to about 2 g/L, about 0.4 to about 120 g/L, about 0.4 to about 100 g/L, about 0.4 to about 75 g/L, about 0.4 to about 60 g/L, about 0.4 to about 50 g/L, about 0.4 to about 40 g/L, about 0.4 to about 30 g/L, about 0.4 to about 15 g/L, about 0.4 to about 7 g/L, about 0.4 to about 5 g/L, about 0.4 to about 2 g/L, about 0.5 to about 1200 g/L, about 0.5 to about 100 g/L, about 0.5 to about 75 g/L, about 0.5 to about 60 g/L, about 0.5 to about 50 g/L, about 0.5 to about 40 g/L, about 0.5 to about 30 g/L, about 0.5 to about 15 g/L, about 0.5 to about 11 g/L, about 0.5 to about 7 g/L, about 0.5 to about 5 g/L, about 0.5 to about 2 g/L, about 50 to about 60 g/L, or about 1 g/L. In some embodiments, the concentration (e.g., concentration in the cell medium) of MVA is equal to or less than about any of 120, 100, 80, 70, 60, 50, 40, 30, 25, 20, 18, 16, 14, 12, 10, 8, 6, 4, 2, 1, 0.8, 0.6, 0.5, 0.4, 0.3, 0.2, or 0.1 g/L

Examples 13-24 also support the use of the compositions and methods disclosed herein to produce large amounts of isoprene. The methods described herein can be used to modify any of the cells and methods of Examples 13-24 to increase the expression level and/or activity level of a mevalonate kinase polypeptide and/or an isoprene synthase polypeptide. Additionally, methods described herein can be used to modify any of the cells and methods of U.S. Ser. No. 61/134,094, filed Jul. 2, 2008 (which is hereby incorporated by reference in its entirety, particularly with respect to methods of making isoprene and isoprene compositions) to increase the expression level and/or activity level of a mevalonate kinase polypeptide and/or an isoprene synthase polypeptide. As discussed above, increasing the expression level and/or activity level of a mevalonate kinase polypeptide and/or an isoprene synthase polypeptide may further increase the production of isoprene.

### Summary of Exemplary Compositions and Methods for Producing Isoprene

This section summaries exemplary compositions and methods for producing isoprene that can be used with cells having increased expression levels and/or activity levels of a mevalonate kinase polypeptide and an isoprene synthase polypeptide. In one aspect, the invention features compositions and methods for the production of isoprene in increased amounts and/or purity. In one aspect, compositions and methods of the invention increase the rate of isoprene production and increase the total amount of isoprene that is produced. For example, cell culture systems that generate 4.8×10⁴nmole/g_{wcm}/hr of isoprene have been produced (Table 1). The efficiency of these systems is demonstrated by the conversion of about 2.2% of the carbon that the cells consume from a cell culture medium into isoprene. As shown in the Examples and Table 2, approximately 3 g of isoprene per liter of broth was generated. If desired, even greater amounts of isoprene can be obtained using other conditions, such as those described herein. In some embodiments, a renewable carbon source is used for the production of isoprene. In some embodiments, the production of isoprene is decoupled from the growth of the cells. In some embodiments, the concentrations of isoprene and any oxidants are within the nonflammable ranges to reduce or eliminate the risk that a fire may occur during production or recovery of isoprene. The compositions and methods of the present invention are desirable because they allow high isoprene yield per cell, high carbon yield, high isoprene purity, high productivity, low energy usage, low production cost and investment, and minimal side reactions. This efficient, large scale, biosynthetic process for isoprene production provides an isoprene source for synthetic isoprene-based rubber and provides a desirable, low-cost alternative to using natural rubber.

As discussed further herein, the amount of isoprene produced by cells can be greatly increased by introducing a heterologous nucleic acid encoding an isoprene synthase polypeptide (e.g., a plant isoprene synthase polypeptide) into the cells. Isoprene synthase polypeptides convert dimethylallyl diphosphate (DMAPP) into isoprene. As shown in the Examples, a heterologous Pueraria Montana (kudzu) isoprene synthase polypeptide was expressed in a variety of host cells, such as Escherichia coli, Panteoa citrea, Bacillus subtilis, Yarrowia lipolytica, and Trichoderma reesei. All of these cells produced more isoprene than the corresponding cells without the heterologous isoprene synthase polypeptide. As illustrated in Tables 1 and 2, large amounts of isoprene are produced using the methods described herein. For example, B. subtilis cells with a heterologous isoprene synthase nucleic acid produced approximately 10-fold more isoprene in a 14 liter fermentor than the corresponding control B. subtilis cells without the heterologous nucleic acid (Table 2). The production of 300 mg of isoprene per liter of broth (mg/L, wherein the volume of broth includes both the volume of the cell medium and the volume of the cells) by E. coli and 30 mg/L by B. subtilis in fermentors indicates that significant amounts of isoprene can be generated (Table 2). If desired, isoprene can be produced on an even larger scale or other conditions described herein can be used to further increase the amount of isoprene. The vectors listed in Tables 1 and 2 and the experimental conditions are described in further detail below and in the Examples section.

TABLE 1
Exemplary yields of isoprene from a shake flask using the cell cultures
and methods of the invention.
The assay for measuring isoprene production is described
in Example 13, part II. For this assay, a sample was removed
at one or more time points from the shake flask and cultured
for 30 minutes. The amount of isoprene produced in this sample
was then measured. The headspace concentration and specific rate of
isoprene production are listed in Table 1 and described further herein.

| | Isoprene Production | |
|---|---|---|
| | in a Headspace vial* | |
| | Headspace | Specific Rate |
| | concentration | μg/L_{broth}/hr/OD |
| Strain | μg/L_{gas} | (nmol/g_{wcm}/hr) |
| E. coli BL21/pTrcKudzu IS | 1.40 | 53.2 |
| | | (781.2) |
| E. coli BL21/pCL DXS yidi Kudzu | 7.61 | 289.1 |
| IS | | (4.25 × 10³) |
| E. coli BL21/MCM127 with kudzu | 23.0 | 874.1 |
| IS and entire MVA pathway | | (12.8 × 10³) |
| E. coli BL21/pET N-HisKudzu IS | 1.49 | 56.6 |
| | | (831.1) |
| Pantoea citrea/pTrcKudzu IS | 0.66 | 25.1 |
| | | (368.6) |
| E. coli w/ Poplar IS | — | 5.6 |
| [Miller (2001)] | | (82.2) |
| Bacillis licheniformis Fall U.S. Pat. No. | — | 4.2 |
| 5,849,970 | | (61.4) |
| Yarrowia lipolytica with kudzu | ~0.05 μg/L | ~2 |
| isoprene synthase | | (~30) |
| Trichoderma reesei with kudzu | ~0.05 μg/L | ~2 |
| isoprene synthase | | (~30) |
| E. coli BL21/pTrcKKD_{y}IₖIS with | 85.9 | 3.2 × 10³ |
| kudzu IS and lower MVA pathway | | (4.8 × 10⁴) |

*Normalized to 1 mL of 1 OD₆₀₀, cultured for 1 hour in a sealed headspace vial with a liquid to headspace volume ratio of 1:19.

TABLE 2
Exemplary yields of isoprene
in a fermentor using the cell cultures and methods of the invention.
The assay for measuring isoprene production is described in Example 13, part II.
For this assay, a sample of the off-gas of the fermentor was taken and analyzed for
the amount of isoprene. The peak headspace concentration (which is the highest
headspace concentration during the fermentation), titer (which is the cumulative,
total amount of isoprene produced per liter of broth), and peak specific rate of
isoprene production (which is the highest specific rate during the fermentation)
are listed in Table 2 and described further herein.

| | Isoprene Production in Fermentors | | |
|---|---|---|---|
| | Peak | | Peak Specific rate |
| | Headspace concentration** | Titer | μg/L_{broth}/hr/OD |
| Strain | (ug/L_{gas}) | (mg/L_{broth}) | (nmol/g_{wcm}/hr) |
| E. coli BL21/pTrcKudzu | 52 | 41.2 | 37 |
| with Kudzu IS | | | (543.3) |
| E. coli FM5/pTrcKudzu | 3 | 3.5 | 21.4 |
| IS | | | (308.1) |
| E. coli BL21/triple strain | 285 | 300 | 240 |
| (DXS, yidi, IS) | | | (3.52 × 10³) |
| E. coli FM5/triple strain | 50.8 | 29 | 180.8 |
| (DXS, yidi, IS) | | | (2.65 × 10³) |
| E. coli/MCM127 with | 3815 | 3044 | 992.5 |
| Kudzu IS and entire MVA | | | (1.46 × 10⁴) |
| pathway | | | |
| E. coli BL21/pCLPtrc | 2418 | 1640 | 1248 |
| UpperPathway gi1.2 | | | (1.83 × 10⁴) |
| integrated lower pathway | | | |
| pTrcKudzu | | | |
| E. coli BL21/MCM401 | 13991 | 23805 | 3733 |
| with 4 × 50uM IPTG | | | (5.49 × 10⁴) |
| E. coli BL21/MCM401 | 22375 | 19541 | 5839.5 |
| with 2 × 100uM IPTG | | | (8.59 × 10⁴) |
| E. coli BL21/pCLPtrc | 3500 | 3300 | 1088 |
| UpperPathwayHGS2- | | | (1.60 × 10⁴) |
| pTrcKKDyIkIS | | | |
| Bacillus subtilis wild-type | 1.5 | 2.5 | 0.8 |
| | | | (11.7) |
| Bacillus pBS Kudzu IS | 16.6 | ~30 | 5 |
| | | (over 100 hrs) | (73.4) |
| Bacillus Marburg 6051 | 2.04 | 0.61 | 24.5 |
| [Wagner and Fall (1999)] | | | (359.8) |
| Bacillus Marburg 6051 | 0.7 | 0.15 | 6.8 |
| Fall U.S. Pat. No. 5,849,970 | | | (100) |

**Normalized to an off-gas flow rate of 1 vvm (1 volume off-gas per 1 L_{broth} per minute).

Additionally, isoprene production by cells that contain a heterologous isoprene synthase nucleic acid can be enhanced by increasing the amount of a 1-deoxy-D-xylulose-5-phosphate synthase (DXS) polypeptide and/or an isopentenyl diphosphate isomerase (IDI) polypeptide expressed by the cells. For example, a DXS nucleic acid and/or an IDI nucleic acid can be introduced into the cells. The DXS nucleic acid may be a heterologous nucleic acid or a duplicate copy of an endogenous nucleic acid. Similarly, the IDI nucleic acid may be a heterologous nucleic acid or a duplicate copy of an endogenous nucleic acid. In some embodiments, the amount of DXS and/or IDI polypeptide is increased by replacing the endogenous DXS and/or IDI promoters or regulatory regions with other promoters and/or regulatory regions that result in greater transcription of the DXS and/or IDI nucleic acids. In some embodiments, the cells contain both a heterologous nucleic acid encoding an isoprene synthase polypeptide (e.g., a plant isoprene synthase nucleic acid) and a duplicate copy of an endogenous nucleic acid encoding an isoprene synthase polypeptide.

The encoded DXS and IDI polypeptides are part of the DXP pathway for the biosynthesis of isoprene (FIG. 19A). DXS polypeptides convert pyruvate and D-glyceraldehyde-3-phosphate into 1-deoxy-D-xylulose-5-phosphate. While not intending to be bound by any particular theory, it is believed that increasing the amount of DXS polypeptide increases the flow of carbon through the DXP pathway, leading to greater isoprene production. IDI polypeptides catalyze the interconversion of isopentenyl diphosphate (IPP) and dimethylallyl diphosphate (DMAPP). While not intending to be bound by any particular theory, it is believed that increasing the amount of IDI polypeptide in cells increases the amount (and conversion rate) of IPP that is converted into DMAPP, which in turn is converted into isoprene.

For example, fermentation of E. coli cells with a kudzu isoprene synthase, S. cerevisia IDI, and E. coli DXS nucleic acids was used to produce isoprene. The levels of isoprene varied from 50 to 300 μg/L over a time period of 15 hours (Example 19, part VII).

In some embodiments, the presence of heterologous or extra endogenous isoprene synthase, IDI, and DXS nucleic acids causes cells to grow more reproducibly or remain viable for longer compared to the corresponding cell with only one or two of these heterologous or extra endogenous nucleic acids. For example, cells containing heterologous isoprene synthase, IDI, and DXS nucleic acids grew better than cells with only heterologous isoprene synthase and DXS nucleic acids or with only a heterologous isoprene synthase nucleic acid. Also, heterologous isoprene synthase, IDI, and DXS nucleic acids were successfully operably linked to a strong promoter on a high copy plasmid that was maintained by E. coli cells, suggesting that large amounts of these polypeptides could be expressed in the cells without causing an excessive amount of toxicity to the cells. While not intending to be bound to a particular theory, it is believed that the presence of heterologous or extra endogenous isoprene synthase and IDI nucleic acids may reduce the amount of one or more potentially toxic intermediates that would otherwise accumulate if only a heterologous or extra endogenous DXS nucleic acid was present in the cells.

In some embodiments, the production of isoprene by cells by cells that contain a heterologous isoprene synthase nucleic acid is augmented by increasing the amount of a MVA pathway polypeptide expressed by the cells (FIGS. 19A and 19B). Exemplary MVA pathways polypeptides include any of the following polypeptides: acetyl-CoA acetyltransferase (AA-CoA thiolase) polypeptides, 3-hydroxy-3-methylglutaryl-CoA synthase (HMG-CoA synthase) polypeptides, 3-hydroxy-3-methylglutaryl-CoA reductase (HMG-CoA reductase) polypeptides, mevalonate kinase (MVK) polypeptides, phosphomevalonate kinase (PMK) polypeptides, diphosphomevalonate decarboxylase (MVD) polypeptides, phosphomevalonate decarboxylase (PMDC) polypeptides, isopentenyl phosphate kinase (IPK) polypeptides, IDI polypeptides, and polypeptides (e.g., fusion polypeptides) having an activity of two or more MVA pathway polypeptides. For example, one or more MVA pathway nucleic acids can be introduced into the cells. In some embodiments, the cells contain the upper MVA pathway, which includes AA-CoA thiolase, HMG-CoA synthase, and HMG-CoA reductase nucleic acids. In some embodiments, the cells contain the lower MVA pathway, which includes MVK, PMK, MVD, and IDI nucleic acids. In some embodiments, the cells contain an entire MVA pathway that includes AA-CoA thiolase, HMG-CoA synthase, HMG-CoA reductase, MVK, PMK, MVD, and IDI nucleic acids. In some embodiments, the cells contain an entire MVA pathway that includes AA-CoA thiolase, HMG-CoA synthase, HMG-CoA reductase, MVK, PMDC, IPK, and IDI nucleic acids. The MVA pathway nucleic acids may be heterologous nucleic acids or duplicate copies of endogenous nucleic acids. In some embodiments, the amount of one or more MVA pathway polypeptides is increased by replacing the endogenous promoters or regulatory regions for the MVA pathway nucleic acids with other promoters and/or regulatory regions that result in greater transcription of the MVA pathway nucleic acids. In some embodiments, the cells contain both a heterologous nucleic acid encoding an isoprene synthase polypeptide (e.g., a plant isoprene synthase nucleic acid) and a duplicate copy of an endogenous nucleic acid encoding an isoprene synthase polypeptide.

For example, E. coli cells containing a nucleic acid encoding a kudzu isoprene synthase polypeptide and nucleic acids encoding Saccharomyces cerevisia MVK, PMK, MVD, and IDI polypeptides generated isoprene at a rate of 6.67×10⁻⁴ mol/L_{broth}/OD₆₀₀/hr (see Example 20). Additionally, a 14 liter fermentation of E. coli cells with nucleic acids encoding Enterococcus faecalis AA-CoA thiolase, HMG-CoA synthase, and HMG-CoA reductase polypeptides produced 22 grams of mevalonic acid (an intermediate of the MVA pathway). A shake flask of these cells produced 2-4 grams of mevalonic acid per liter. These results indicate that heterologous MVA pathways nucleic acids are active in E. coli. E. coli cells that contain nucleic acids for both the upper MVA pathway and the lower MVA pathway as well as a kudzu isoprene synthase (strain MCM 127) produced significantly more isoprene (874 ug/L) compared to E. coli cells with nucleic acids for only the lower MVA pathway and the kudzu isoprene synthase (strain MCM 131) (see Table 10 and Example 20, part VIII).

In some embodiments, at least a portion of the cells maintain the heterologous isoprene synthase, DXS, IDI, and/or MVA pathway nucleic acid for at least about 5, 10, 20, 50, 75, 100, 200, 300, or more cell divisions in a continuous culture (such as a continuous culture without dilution). In some embodiments of any of the aspects of the invention, the nucleic acid comprising the heterologous or duplicate copy of an endogenous isoprene synthase, DXS, IDI, and/or MVA pathway nucleic acid also comprises a selective marker, such as a kanamycin, ampicillin, carbenicillin, gentamicin, hygromycin, phleomycin, bleomycin, neomycin, or chloramphenicol antibiotic resistance nucleic acid.

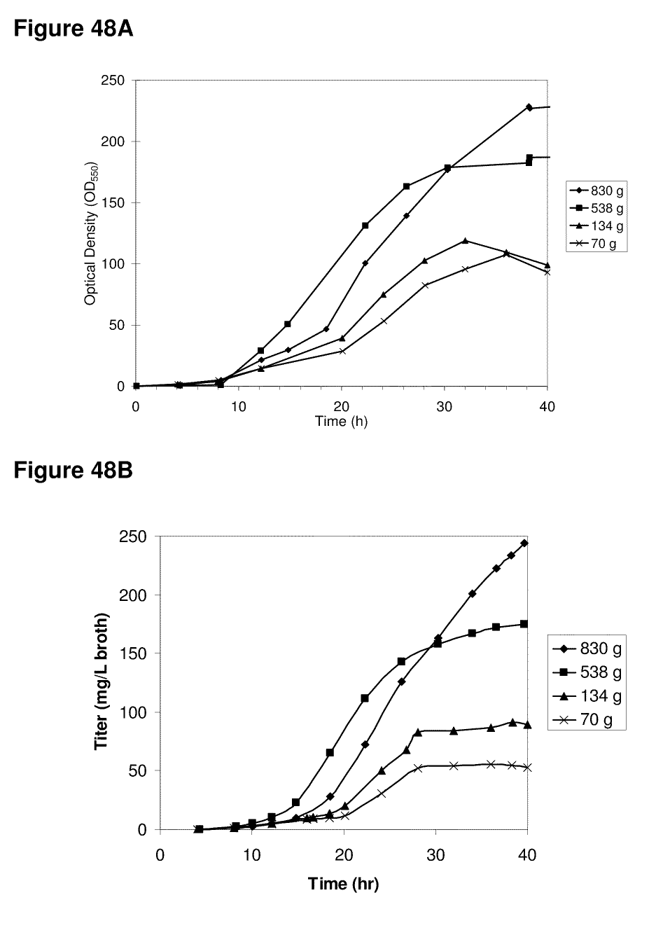

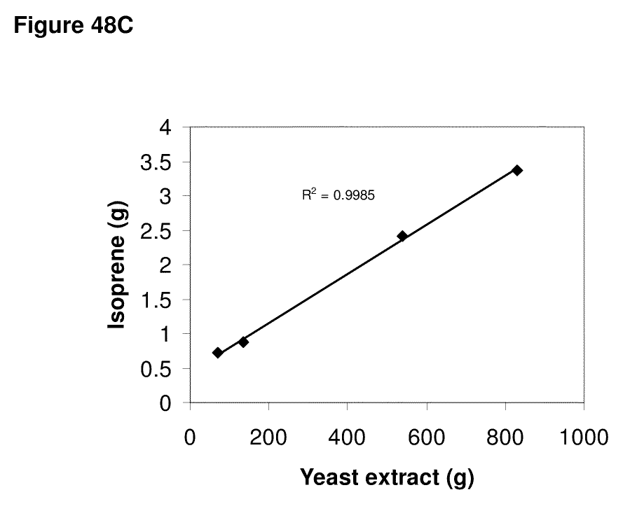

As indicated in Example 19, part VI, the amount of isoprene produced can be further increased by adding yeast extract to the cell culture medium. In this example, the amount of isoprene produced was linearly proportional to the amount of yeast extract in the cell medium for the concentrations tested (FIG. 48C). Additionally, approximately 0.11 grams of isoprene per liter of broth was produced from a cell medium with yeast extract and glucose (Example 19, part VIII). Both of these experiments used E. coli cells with kudzu isoprene synthase, S. cerevisia IDI, and E. coli DXS nucleic acids to produce isoprene. Increasing the amount of yeast extract in the presence of glucose resulted in more isoprene being produced than increasing the amount of glucose in the presence of yeast extract. Also, increasing the amount of yeast extract allowed the cells to produce a high level of isoprene for a longer length of time and improved the health of the cells.

Isoprene production was also demonstrated using three types of hydrolyzed biomass (bagasse, corn stover, and soft wood pulp) as the carbon source. E. coli cells with kudzu isoprene synthase, S. cerevisia IDI, and E. coli DXS nucleic acids produced as much isoprene from these hydrolyzed biomass carbon sources as from the equivalent amount of glucose (e.g., 1% glucose, w/v). If desired, any other biomass carbon source can be used in the compositions and methods of the invention. Biomass carbon sources are desirable because they are cheaper than many conventional cell mediums, thereby facilitating the economical production of isoprene.

Additionally, invert sugar was shown to function as a carbon source for the generation of isoprene. For example, 2.4 g/L of isoprene was produced from cells expressing MVA pathway polypeptides and a Kudzu isoprene synthase. Glycerol was as also used as a carbon source for the generation of 2.2 mg/L of isoprene from cells expressing a Kudzu isoprene synthase. Expressing a DXS nucleic acid, an IDI nucleic acid, and/or one or more MVA pathway nucleic acids (such as nucleic acids encoding the entire MVA pathway) in addition to an isoprene synthase nucleic acid may increase the production of isoprene from glycerol.

In some embodiments, an oil is included in the cell medium. For example, B. subtilis cells containing a kudzu isoprene synthase nucleic acid produced isoprene when cultured in a cell medium containing an oil and a source of glucose (Example 16, part III). In some embodiments, more than one oil (such as 2, 3, 4, 5, or more oils) is included in the cell medium. While not intending to be bound to any particular theory, it is believed that (i) the oil may increase the amount of carbon in the cells that is available for conversion to isoprene, (ii) the oil may increase the amount of acetyl-CoA in the cells, thereby increasing the carbon flow through the MVA pathway, and/or (ii) the oil may provide extra nutrients to the cells, which is desirable since a lot of the carbon in the cells is converted to isoprene rather than other products. In some embodiments, cells that are cultured in a cell medium containing oil naturally use the MVA pathway to produce isoprene or are genetically modified to contain nucleic acids for the entire MVA pathway. In some embodiments, the oil is partially or completely hydrolyzed before being added to the cell culture medium to facilitate the use of the oil by the host cells.

One of the major hurdles to commercial production of small molecules such as isoprene in cells (e.g., bacteria) is the decoupling of production of the molecule from growth of the cells. In some embodiments for the commercially viable production of isoprene, a significant amount of the carbon from the feedstock is converted to isoprene, rather than to the growth and maintenance of the cells (“carbon efficiency”). In various embodiments, the cells convert greater than or about 0.0015, 0.002, 0.005, 0.01, 0.02, 0.05, 0.1, 0.12, 0.14, 0.16, 0.2, 0.3, 0.4, 0.5, 0.6, 0.7, 0.8, 0.9, 1.0, 1.2, 1.4, 1.6, 1.8, 2.0, 2.5, 3.0, 3.5, 4.0, 5.0, 6.0, 7.0, or 8.0% of the carbon in the cell culture medium into isoprene. In particular embodiments, a significant portion of the carbon from the feedstock that is converted to downstream products is converted to isoprene. As described further in Example 22, E. coli cells expressing MVA pathway and kudzu isoprene synthase nucleic acids exhibited decoupling of the production of isoprene or the intermediate mevalonic acid from growth, resulting in high carbon efficiency. In particular, mevalonic acid was formed from cells expressing the upper MVA pathway from Enterococcus faecalis. Isoprene was formed from cells expressing the upper MVA pathway from Enterococcus faecalis, the lower MVA pathway from Saccharomyces cerevisiae, and the isoprene synthase from Pueraria montana (Kudzu). This decoupling of isoprene or mevalonic acid production from growth was demonstrated in four different strains of E. coli: BL21(LDE3), BL21(LDE3) Tuner, FM5, and MG1655. The first two E. coli strains are B strains, and the latter two are K12 strains. Decoupling of production from growth was also demonstrated in a variant of MG1655 with ack and pta genes deleted. This variant also demonstrated less production of acetate.

The vast majority of isoprene is derived from petrochemical sources as an impure C5 hydrocarbon fraction which requires extensive purification before the material is suitable for polymerization. Several impurities are particularly problematic given their structural similarity to isoprene and the fact that they can act as polymerization catalyst poisons. Such compounds include 1,3-cyclopentadiene, trans-1,3-pentadiene, cis-1,3-pentadiene, 1,4-pentadiene, 1-pentyne, 2-pentyne, 3-methyl-1-butyne, pent-4-ene-1-yne, trans-pent-3-ene-1-yne, cis-pent-3-ene-1-yne, 3-hexen-1-ol, 3-hexen-1-yl acetate, limonene, geraniol (trans-3,7-dimethyl-2,6-octadien-1-ol) and citronellol (3,7-dimethyl-6-octen-1-ol).

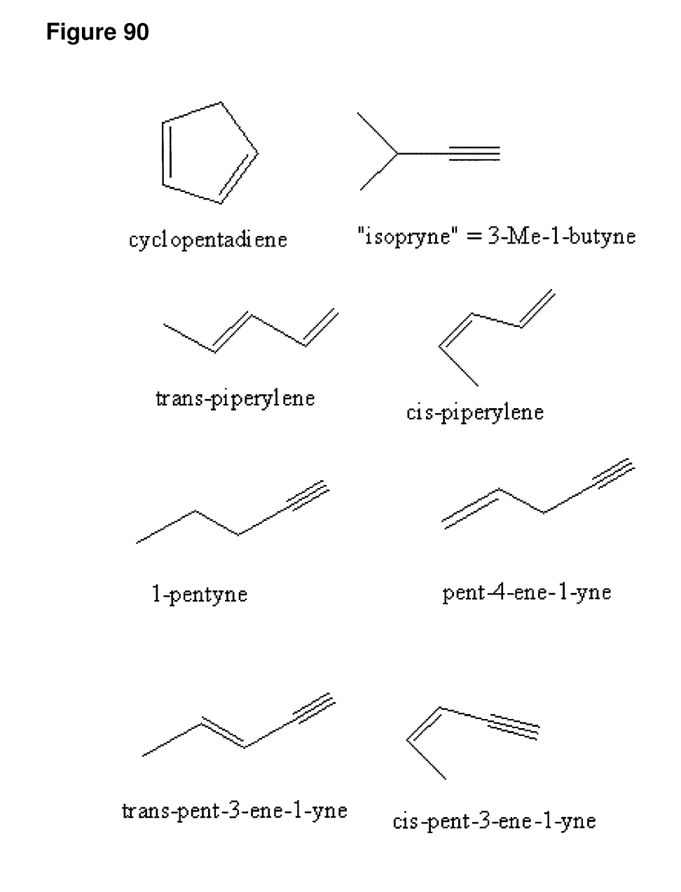

(FIG. 90). In some embodiments, the isoprene composition of the invention is substantially free of any contaminating unsaturated C5 hydrocarbons. No detectable amount of unsaturated C5 hydrocarbons other than isoprene (such as 1,3-cyclopentadiene, trans-1,3-pentadiene, cis-1,3-pentadiene, 1,4-pentadiene, 1-pentyne, 2-pentyne, 3-methyl-1-butyne, pent-4-ene-1-yne, trans-pent-3-ene-1-yne, cis-pent-3-ene-1-yne, 3-hexen-1-ol, 3-hexen-1-yl acetate, limonene, geraniol (trans-3,7-dimethyl-2,6-octadien-1-ol) and citronellol (3,7-dimethyl-6-octen-1-ol)) was found in isoprene compositions produced using the methods described herein. Some isoprene compositions produced using the methods described herein contain ethanol, acetone, and C5 prenyl alcohols as determined by GC/MS analysis. All of these components are far more readily removed from the isoprene stream than the isomeric C5 hydrocarbon fractions that are present in isoprene compositions derived from petrochemical sources. Accordingly, in some embodiments, the isoprene compositions of the invention require minimal treatment in order to be of polymerization grade.

### Exemplary Polypeptides and Nucleic Acids

Various isoprene synthase, DXS, IDI, and/or MVA pathway polypeptides and nucleic acids can be used in the compositions and methods of the invention.

As used herein, “polypeptides” includes polypeptides, proteins, peptides, fragments of polypeptides, and fusion polypeptides. In some embodiments, the fusion polypeptide includes part or all of a first polypeptide (e.g., an isoprene synthase, DXS, IDI, or MVA pathway polypeptide or catalytically active fragment thereof) and may optionally include part or all of a second polypeptide (e.g., a peptide that facilitates purification or detection of the fusion polypeptide, such as a His-tag). In some embodiments, the fusion polypeptide has an activity of two or more MVA pathway polypeptides (such as AA-CoA thiolase and HMG-CoA reductase polypeptides). In some embodiments, the polypeptide is a naturally-occurring polypeptide (such as the polypeptide encoded by an Enterococcus faecalis mvaE nucleic acid) that has an activity of two or more MVA pathway polypeptides.

In various embodiments, a polypeptide has at least or about 50, 100, 150, 175, 200, 250, 300, 350, 400, or more amino acids. In some embodiments, the polypeptide fragment contains at least or about 25, 50, 75, 100, 150, 200, 300, or more contiguous amino acids from a full-length polypeptide and has at least or about 5%, 10%, 20%, 30%, 40%, 50%, 60%, 70%, 80%, 90%, 95%, or 100% of an activity of a corresponding full-length polypeptide. In particular embodiments, the polypeptide includes a segment of or the entire amino acid sequence of any naturally-occurring isoprene synthase, DXS, IDI, or MVA pathway polypeptide. In some embodiments, the polypeptide has one or more mutations compared to the sequence of a wild-type (i.e., a sequence occurring in nature) isoprene synthase, DXS, IDI, or MVA pathway polypeptide.

In some embodiments, the polypeptide is an isolated polypeptide. As used herein, an “isolated polypeptide” is not part of a library of polypeptides, such as a library of 2, 5, 10, 20, 50 or more different polypeptides and is separated from at least one component with which it occurs in nature. An isolated polypeptide can be obtained, for example, by expression of a recombinant nucleic acid encoding the polypeptide.

In some embodiments, the polypeptide is a heterologous polypeptide. By “heterologous polypeptide” is meant a polypeptide whose amino acid sequence is not identical to that of another polypeptide naturally expressed in the same host cell. In particular, a heterologous polypeptide is not identical to a wild-type nucleic acid that is found in the same host cell in nature.

As used herein, a “nucleic acid” refers to two or more deoxyribonucleotides and/or ribonucleotides in either single or double-stranded form. In some embodiments, the nucleic acid is a recombinant nucleic acid. By “recombinant nucleic acid” means a nucleic acid of interest that is free of one or more nucleic acids (e.g., genes) which, in the genome occurring in nature of the organism from which the nucleic acid of interest is derived, flank the nucleic acid of interest. The term therefore includes, for example, a recombinant DNA which is incorporated into a vector, into an autonomously replicating plasmid or virus, or into the genomic DNA of a prokaryote or eukaryote, or which exists as a separate molecule (e.g., a cDNA, a genomic DNA fragment, or a cDNA fragment produced by PCR or restriction endonuclease digestion) independent of other sequences. In various embodiments, a nucleic acid is a recombinant nucleic acid. In some embodiments, an isoprene synthase, DXS, IDI, or MVA pathway nucleic acid is operably linked to another nucleic acid encoding all or a portion of another polypeptide such that the recombinant nucleic acid encodes a fusion polypeptide that includes an isoprene synthase, DXS, IDI, or MVA pathway polypeptide and all or part of another polypeptide (e.g., a peptide that facilitates purification or detection of the fusion polypeptide, such as a His-tag). In some embodiments, part or all of a recombinant nucleic acid is chemically synthesized. It is to be understood that mutations, including single nucleotide mutations, can occur within a nucleic acid as defined herein.

In some embodiments, the nucleic acid is a heterologous nucleic acid. By “heterologous nucleic acid” is meant a nucleic acid whose nucleic acid sequence is not identical to that of another nucleic acid naturally found in the same host cell.

In particular embodiments, the nucleic acid includes a segment of or the entire nucleic acid sequence of any naturally-occurring isoprene synthase, DXS, IDI, or MVA pathway nucleic acid. In some embodiments, the nucleic acid includes at least or about 50, 100, 150, 200, 300, 400, 500, 600, 700, 800, or more contiguous nucleotides from a naturally-occurring isoprene synthase nucleic acid DXS, IDI, or MVA pathway nucleic acid. In some embodiments, the nucleic acid has one or more mutations compared to the sequence of a wild-type (i.e., a sequence occurring in nature) isoprene synthase, DXS, IDI, or MVA pathway nucleic acid. In some embodiments, the nucleic acid has one or more mutations (e.g., a silent mutation) that increase the transcription or translation of isoprene synthase, DXS, IDI, or MVA pathway nucleic acid. In some embodiments, the nucleic acid is a degenerate variant of any nucleic acid encoding an isoprene synthase, DXS, IDI, or MVA pathway polypeptide.

“Codon degeneracy” refers to divergence in the genetic code permitting variation of the nucleotide sequence without affecting the amino acid sequence of an encoded polypeptide. The skilled artisan is well aware of the “codon-bias” exhibited by a specific host cell in usage of nucleotide codons to specify a given amino acid. Therefore, when synthesizing a nucleic acid for improved expression in a host cell, it is desirable in some embodiments to design the nucleic acid such that its frequency of codon usage approaches the frequency of preferred codon usage of the host cell.

The accession numbers of exemplary isoprene synthase, DXS, IDI, and/or MVA pathway polypeptides and nucleic acids are listed in Appendix 1 (the accession numbers of Appendix 1 and their corresponding sequences are herein incorporated by reference in their entireties, particularly with respect to the amino acid and nucleic acid sequences of isoprene synthase, DXS, IDI, and/or MVA pathway polypeptides and nucleic acids). The Kegg database also contains the amino acid and nucleic acid sequences of numerous exemplary isoprene synthase, DXS, IDI, and/or MVA pathway polypeptides and nucleic acids (see, for example, the world-wide web at “genome.jp/kegg/pathway/map/map00100.html” and the sequences therein, which are each hereby incorporated by reference in their entireties, particularly with respect to the amino acid and nucleic acid sequences of isoprene synthase, DXS, IDI, and/or MVA pathway polypeptides and nucleic acids). In some embodiments, one or more of the isoprene synthase, DXS, IDI, and/or MVA pathway polypeptides and/or nucleic acids have a sequence identical to a sequence publicly available on Dec. 12, 2007 or Sep. 14, 2008, such as any of the sequences that correspond to any of the accession numbers in Appendix 1 or any of the sequences present in the Kegg database. Additional exemplary isoprene synthase, DXS, IDI, and/or MVA pathway polypeptides and nucleic acids are described further below.

### Exemplary Isoprene Synthase Polypeptides and Nucleic Acids

As noted above, isoprene synthase polypeptides convert dimethylallyl diphosphate (DMAPP) into isoprene. Exemplary isoprene synthase polypeptides include polypeptides, fragments of polypeptides, peptides, and fusions polypeptides that have at least one activity of an isoprene synthase polypeptide. Standard methods can be used to determine whether a polypeptide has isoprene synthase polypeptide activity by measuring the ability of the polypeptide to convert DMAPP into isoprene in vitro, in a cell extract, or in vivo. In an exemplary assay, cell extracts are prepared by growing a strain (e.g., the E. coli/pTrcKudzu strain described herein) in the shake flask method as described in Example 13. After induction is complete, approximately 10 mL of cells are pelleted by centrifugation at 7000×g for 10 minutes and resuspended in 5 ml of PEB without glycerol. The cells are lysed using a French Pressure cell using standard procedures. Alternatively the cells are treated with lysozyme (Ready-Lyse lysozyme solution; EpiCentre) after a freeze/thaw at −80 C.

Isoprene synthase polypeptide activity in the cell extract can be measured, for example, as described in Silver et al., J. Biol. Chem. 270:13010-13016, 1995 and references therein, which are each hereby incorporated by reference in their entireties, particularly with respect to assays for isoprene synthase polypeptide activity. DMAPP (Sigma) is evaporated to dryness under a stream of nitrogen and rehydrated to a concentration of 100 mM in 100 mM potassium phosphate buffer pH 8.2 and stored at −20° C. To perform the assay, a solution of 5 μL of 1M MgCl₂, 1 mM (25 μg/ml) DMAPP, 65 μL of Plant Extract Buffer (PEB) (50 mM Tris-HCl, pH 8.0, 20 mM MgCl₂, 5% glycerol, and 2 mM DTT) is added to 25 μL of cell extract in a 20 ml Headspace vial with a metal screw cap and teflon coated silicon septum (Agilent Technologies) and cultured at 37° C. for 15 minutes with shaking. The reaction is quenched by adding 200 μL of 250 mM EDTA and quantified by GC/MS as described in Example 13, part II.

Exemplary isoprene synthase nucleic acids include nucleic acids that encode a polypeptide, fragment of a polypeptide, peptide, or fusion polypeptide that has at least one activity of an isoprene synthase polypeptide. Exemplary isoprene synthase polypeptides and nucleic acids include naturally-occurring polypeptides and nucleic acids from any of the source organisms described herein as well as mutant polypeptides and nucleic acids derived from any of the source organisms described herein.

In some embodiments, the isoprene synthase polypeptide or nucleic acid is from the family Fabaceae, such as the Faboideae subfamily. In some embodiments, the isoprene synthase polypeptide or nucleic acid is a polypeptide or nucleic acid from Pueraria montana (kudzu) (Sharkey et al., Plant Physiology 137: 700-712, 2005), Pueraria lobata, poplar (such as Populus alba, Populus nigra, Populus trichocarpa, or Populus alba x tremula (CAC35696) Miller et al., Planta 213: 483-487, 2001) aspen (such as Populus tremuloides) Silver et al., JBC 270(22): 13010-1316, 1995), or English Oak (Quercus robur) (Zimmer et al., WO 98/02550), which are each hereby incorporated by reference in their entireties, particularly with respect to isoprene synthase nucleic acids and the expression of isoprene synthase polypeptides. Suitable isoprene synthases include, but are not limited to, those identified by Genbank Accession Nos. AY341431, AY316691, AY279379, AJ457070, and AY182241, which are each hereby incorporated by reference in their entireties, particularly with respect to sequences of isoprene synthase nucleic acids and polypeptides. In some embodiments, the isoprene synthase polypeptide or nucleic acid is not a naturally-occurring polypeptide or nucleic acid from Quercus robur (i.e., the isoprene synthase polypeptide or nucleic acid is an isoprene synthase polypeptide or nucleic acid other than a naturally-occurring polypeptide or nucleic acid from Quercus robur). In some embodiments, the isoprene synthase nucleic acid or polypeptide is a naturally-occurring polypeptide or nucleic acid from poplar. In some embodiments, the isoprene synthase nucleic acid or polypeptide is not a naturally-occurring polypeptide or nucleic acid from poplar.

### Exemplary DXS Polypeptides and Nucleic Acids

As noted above, 1-deoxy-D-xylulose-5-phosphate synthase (DXS) polypeptides convert pyruvate and D-glyceraldehyde-3-phosphate into 1-deoxy-D-xylulose-5-phosphate. Exemplary DXS polypeptides include polypeptides, fragments of polypeptides, peptides, and fusions polypeptides that have at least one activity of a DXS polypeptide. Standard methods (such as those described herein) can be used to determine whether a polypeptide has DXS polypeptide activity by measuring the ability of the polypeptide to convert pyruvate and D-glyceraldehyde-3-phosphate into 1-deoxy-D-xylulose-5-phosphate in vitro, in a cell extract, or in vivo. Exemplary DXS nucleic acids include nucleic acids that encode a polypeptide, fragment of a polypeptide, peptide, or fusion polypeptide that has at least one activity of a DXS polypeptide. Exemplary DXS polypeptides and nucleic acids include naturally-occurring polypeptides and nucleic acids from any of the source organisms described herein as well as mutant polypeptides and nucleic acids derived from any of the source organisms described herein.

### Exemplary IDI Polypeptides and Nucleic Acids

Isopentenyl diphosphate isomerase polypeptides (isopentenyl-diphosphate delta-isomerase or IDI) catalyses the interconversion of isopentenyl diphosphate (IPP) and dimethylallyl diphosphate (DMAPP) (e.g., converting IPP into DMAPP and/or converting DMAPP into IPP). Exemplary IDI polypeptides include polypeptides, fragments of polypeptides, peptides, and fusions polypeptides that have at least one activity of an IDI polypeptide. Standard methods (such as those described herein) can be used to determine whether a polypeptide has IDI polypeptide activity by measuring the ability of the polypeptide to interconvert IPP and DMAPP in vitro, in a cell extract, or in vivo. Exemplary IDI nucleic acids include nucleic acids that encode a polypeptide, fragment of a polypeptide, peptide, or fusion polypeptide that has at least one activity of an IDI polypeptide. Exemplary IDI polypeptides and nucleic acids include naturally-occurring polypeptides and nucleic acids from any of the source organisms described herein as well as mutant polypeptides and nucleic acids derived from any of the source organisms described herein.

### Exemplary MVA Pathway Polypeptides and Nucleic Acids

Exemplary MVA pathway polypeptides include acetyl-CoA acetyltransferase (AA-CoA thiolase) polypeptides, 3-hydroxy-3-methylglutaryl-CoA synthase (HMG-CoA synthase) polypeptides, 3-hydroxy-3-methylglutaryl-CoA reductase (HMG-CoA reductase) polypeptides, mevalonate kinase (MVK) polypeptides, phosphomevalonate kinase (PMK) polypeptides, diphosphomevalonate decarboxylase (MVD) polypeptides, phosphomevalonate decarboxylase (PMDC) polypeptides, isopentenyl phosphate kinase (IPK) polypeptides, IDI polypeptides, and polypeptides (e.g., fusion polypeptides) having an activity of two or more MVA pathway polypeptides. In particular, MVA pathway polypeptides include polypeptides, fragments of polypeptides, peptides, and fusions polypeptides that have at least one activity of an MVA pathway polypeptide. Exemplary MVA pathway nucleic acids include nucleic acids that encode a polypeptide, fragment of a polypeptide, peptide, or fusion polypeptide that has at least one activity of an MVA pathway polypeptide. Exemplary MVA pathway polypeptides and nucleic acids include naturally-occurring polypeptides and nucleic acids from any of the source organisms described herein as well as mutant polypeptides and nucleic acids derived from any of the source organisms described herein.

In particular, acetyl-CoA acetyltransferase polypeptides (AA-CoA thiolase or AACT) convert two molecules of acetyl-CoA into acetoacetyl-CoA. Standard methods (such as those described herein) can be used to determine whether a polypeptide has AA-CoA thiolase polypeptide activity by measuring the ability of the polypeptide to convert two molecules of acetyl-CoA into acetoacetyl-CoA in vitro, in a cell extract, or in vivo.

3-hydroxy-3-methylglutaryl-CoA synthase (HMG-CoA synthase or HMGS) polypeptides convert acetoacetyl-CoA into 3-hydroxy-3-methylglutaryl-CoA. Standard methods (such as those described herein) can be used to determine whether a polypeptide has HMG-CoA synthase polypeptide activity by measuring the ability of the polypeptide to convert acetoacetyl-CoA into 3-hydroxy-3-methylglutaryl-CoA in vitro, in a cell extract, or in vivo.

3-hydroxy-3-methylglutaryl-CoA reductase (HMG-CoA reductase or HMGR) polypeptides convert 3-hydroxy-3-methylglutaryl-CoA into mevalonate. Standard methods (such as those described herein) can be used to determine whether a polypeptide has HMG-CoA reductase polypeptide activity by measuring the ability of the polypeptide to convert 3-hydroxy-3-methylglutaryl-CoA into mevalonate in vitro, in a cell extract, or in vivo.

Mevalonate kinase (MVK) polypeptides phosphorylates mevalonate to form mevalonate-5-phosphate. Standard methods (such as those described herein) can be used to determine whether a polypeptide has MVK polypeptide activity by measuring the ability of the polypeptide to convert mevalonate into mevalonate-5-phosphate in vitro, in a cell extract, or in vivo.

Phosphomevalonate kinase (PMK) polypeptides phosphorylates mevalonate-5-phosphate to form mevalonate-5-diphosphate. Standard methods (such as those described herein) can be used to determine whether a polypeptide has PMK polypeptide activity by measuring the ability of the polypeptide to convert mevalonate-5-phosphate into mevalonate-5-diphosphate in vitro, in a cell extract, or in vivo.

Diphosphomevalonate decarboxylase (MVD or DPMDC) polypeptides convert mevalonate-5-diphosphate into isopentenyl diphosphate (IPP). Standard methods (such as those described herein) can be used to determine whether a polypeptide has MVD polypeptide activity by measuring the ability of the polypeptide to convert mevalonate-5-diphosphate into IPP in vitro, in a cell extract, or in vivo.

Phosphomevalonate decarboxylase (PMDC) polypeptides convert mevalonate-5-phosphate into isopentenyl phosphate (IP). Standard methods (such as those described herein) can be used to determine whether a polypeptide has PMDC polypeptide activity by measuring the ability of the polypeptide to convert mevalonate-5-phosphate into IP in vitro, in a cell extract, or in vivo.

Isopentenyl phosphate kinase (IPK) polypeptides phosphorylate isopentyl phosphate (IP) to form isopentenyl diphosphate (IPP). Standard methods (such as those described herein) can be used to determine whether a polypeptide has IPK polypeptide activity by measuring the ability of the polypeptide to convert IP into IPP in vitro, in a cell extract, or in vivo.

Exemplary IDI polypeptides and nucleic acids are described above.

### Exemplary Methods for Isolating Nucleic Acids

Isoprene synthase, DXS, IDI, and/or MVA pathway nucleic acids can be isolated using standard methods. Methods of obtaining desired nucleic acids from a source organism of interest (such as a bacterial genome) are common and well known in the art of molecular biology (see, for example, WO 2004/033646 and references cited therein, which are each hereby incorporated by reference in their entireties, particularly with respect to the isolation of nucleic acids of interest). For example, if the sequence of the nucleic acid is known (such as any of the known nucleic acids described herein), suitable genomic libraries may be created by restriction endonuclease digestion and may be screened with probes complementary to the desired nucleic acid sequence. Once the sequence is isolated, the DNA may be amplified using standard primer directed amplification methods such as polymerase chain reaction (PCR) (U.S. Pat. No. 4,683,202, which is incorporated by reference in its entirety, particularly with respect to PCR methods) to obtain amounts of DNA suitable for transformation using appropriate vectors.

Alternatively, isoprene synthase, DXS, IDI, and/or MVA pathway nucleic acids (such as any isoprene synthase, DXS, IDI, and/or MVA pathway nucleic acids with a known nucleic acid sequence) can be chemically synthesized using standard methods.

Additional isoprene synthase, DXS, IDI, or MVA pathway polypeptides and nucleic acids which may be suitable for use in the compositions and methods described herein can be identified using standard methods. For example, cosmid libraries of the chromosomal DNA of organisms known to produce isoprene naturally can be constructed in organisms such as E. coli, and then screened for isoprene production. In particular, cosmid libraries may be created where large segments of genomic DNA (35-45 kb) are packaged into vectors and used to transform appropriate hosts. Cosmid vectors are unique in being able to accommodate large quantities of DNA. Generally cosmid vectors have at least one copy of the cos DNA sequence which is needed for packaging and subsequent circularization of the heterologous DNA. In addition to the cos sequence, these vectors also contain an origin of replication such as Co1EI and drug resistance markers such as a nucleic acid resistant to ampicillin or neomycin. Methods of using cosmid vectors for the transformation of suitable bacterial hosts are well described in Sambrook et al., Molecular Cloning: A Laboratory Manual, 2^{nd} ed., Cold Spring Harbor, 1989, which is hereby incorporated by reference in its entirety, particularly with respect to transformation methods.

Typically to clone cosmids, heterologous DNA is isolated using the appropriate restriction endonucleases and ligated adjacent to the cos region of the cosmid vector using the appropriate ligases. Cosmid vectors containing the linearized heterologous DNA are then reacted with a DNA packaging vehicle such as bacteriophage. During the packaging process, the cos sites are cleaved and the heterologous DNA is packaged into the head portion of the bacterial viral particle. These particles are then used to transfect suitable host cells such as E. coli. Once injected into the cell, the heterologous DNA circularizes under the influence of the cos sticky ends. In this manner, large segments of heterologous DNA can be introduced and expressed in host cells.

Additional methods for obtaining isoprene synthase, DXS, IDI, and/or MVA pathway nucleic acids include screening a metagenomic library by assay (such as the headspace assay described herein) or by PCR using primers directed against nucleotides encoding for a length of conserved amino acids (for example, at least 3 conserved amino acids). Conserved amino acids can be identified by aligning amino acid sequences of known isoprene synthase, DXS, IDI, and/or MVA pathway polypeptides. Conserved amino acids for isoprene synthase polypeptides can be identified based on aligned sequences of known isoprene synthase polypeptides. An organism found to produce isoprene naturally can be subjected to standard protein purification methods (which are well known in the art) and the resulting purified polypeptide can be sequenced using standard methods. Other methods are found in the literature (see, for example, Julsing et al., Applied. Microbiol. Biotechnol. 75: 1377-84, 2007; Withers et al., Appl Environ Microbiol. 73(19):6277-83, 2007, which are each hereby incorporated by reference in their entireties, particularly with respect to identification of nucleic acids involved in the synthesis of isoprene).

Additionally, standard sequence alignment and/or structure prediction programs can be used to identify additional DXS, IDI, or MVA pathway polypeptides and nucleic acids based on the similarity of their primary and/or predicted polypeptide secondary structure with that of known DXS, IDI, or MVA pathway polypeptides and nucleic acids. Standard databases such as the swissprot-trembl database (world-wide web at “expasy.org”, Swiss Institute of Bioinformatics Swiss-Prot group CMU - 1 rue Michel Servet CH-1211 Geneva 4, Switzerland) can also be used to identify isoprene synthase, DXS, IDI, or MVA pathway polypeptides and nucleic acids. The secondary and/or tertiary structure of an isoprene synthase, DXS, IDI, or MVA pathway polypeptide can be predicted using the default settings of standard structure prediction programs, such as PredictProtein (630 West, 168 Street, BB217, New York, N.Y. 10032, USA). Alternatively, the actual secondary and/or tertiary structure of an isoprene synthase, DXS, IDI, or MVA pathway polypeptide can be determined using standard methods. Additional isoprene synthase, DXS, IDI, or MVA pathway nucleic acids can also be identified by hybridization to probes generated from known isoprene synthase, DXS, IDI, or MVA pathway nucleic acids.

### Exemplary Promoters and Vectors

Any of the isoprene synthase, DXS, IDI, or MVA pathway nucleic acid described herein can be included in one or more vectors. Accordingly, the invention also features vectors with one more nucleic acids encoding any of the isoprene synthase, DXS, IDI, or MVA pathway polypeptides that are described herein. As used herein, a “vector” means a construct that is capable of delivering, and desirably expressing one or more nucleic acids of interest in a host cell. Examples of vectors include, but are not limited to, plasmids, viral vectors, DNA or RNA expression vectors, cosmids, and phage vectors. In some embodiments, the vector contains a nucleic acid under the control of an expression control sequence.

As used herein, an “expression control sequence” means a nucleic acid sequence that directs transcription of a nucleic acid of interest. An expression control sequence can be a promoter, such as a constitutive or an inducible promoter, or an enhancer. An “inducible promoter” is a promoter that is active under environmental or developmental regulation. The expression control sequence is operably linked to the nucleic acid segment to be transcribed.

In some embodiments, the vector contains a selective marker. The term “selective marker” refers to a nucleic acid capable of expression in a host cell that allows for ease of selection of those host cells containing an introduced nucleic acid or vector. Examples of selectable markers include, but are not limited to, antibiotic resistance nucleic acids (e.g., kanamycin, ampicillin, carbenicillin, gentamicin, hygromycin, phleomycin, bleomycin, neomycin, or chloramphenicol) and/or nucleic acids that confer a metabolic advantage, such as a nutritional advantage on the host cell. Exemplary nutritional selective markers include those markers known in the art as amdS, argB, and pyr4. Markers useful in vector systems for transformation of Trichoderma are known in the art (see, e.g., Finkelstein, Chapter 6 in Biotechnology of Filamentous Fungi, Finkelstein et al., Eds. Butterworth-Heinemann, Boston, Mass., Chap. 6., 1992; and Kinghorn et al., Applied Molecular Genetics of Filamentous Fungi, Blackie Academic and Professional, Chapman and Hall, London, 1992, which are each hereby incorporated by reference in their entireties, particularly with respect to selective markers). In some embodiments, the selective marker is the amdS nucleic acid, which encodes the enzyme acetamidase, allowing transformed cells to grow on acetamide as a nitrogen source. The use of an A. nidulans amdS nucleic acid as a selective marker is described in Kelley et al., EMBO J. 4:475-479, 1985 and Penttila et al., Gene 61:155-164, 1987 (which are each hereby incorporated by reference in their entireties, particularly with respect to selective markers). In some embodiments, an isoprene synthase, DXS, IDI, or MVA pathway nucleic acid integrates into a chromosome of the cells without a selective marker.

Suitable vectors are those which are compatible with the host cell employed. Suitable vectors can be derived, for example, from a bacterium, a virus (such as bacteriophage T7 or a M-13 derived phage), a cosmid, a yeast, or a plant. Protocols for obtaining and using such vectors are known to those in the art (see, for example, Sambrook et al., Molecular Cloning: A Laboratory Manual, 2^{nd} ed., Cold Spring Harbor, 1989, which is hereby incorporated by reference in its entirety, particularly with respect to the use of vectors).

Promoters are well known in the art. Any promoter that functions in the host cell can be used for expression of an isoprene synthase, DXS, IDI, or MVA pathway nucleic acid in the host cell. Initiation control regions or promoters, which are useful to drive expression of isoprene synthase, DXS, IDI, or MVA pathway nucleic acids in various host cells are numerous and familiar to those skilled in the art (see, for example, WO 2004/033646 and references cited therein, which are each hereby incorporated by reference in their entireties, particularly with respect to vectors for the expression of nucleic acids of interest). Virtually any promoter capable of driving these nucleic acids is suitable for the present invention including, but not limited to, CYC1, HIS3, GAL1, GAL10, ADH1, PGK, PHO5, GAPDH, ADCI, TRP1, URA3, LEU2, ENO, and TPI (useful for expression in Saccharomyces); AOX1 (useful for expression in Pichia); and lac, trp, λP_{L}, λP_{R}, T7, tac, and trc (useful for expression in E. coli).

In some embodiments, a glucose isomerase promoter is used (see, for example, U.S. Pat. No. 7,132,527 and references cited therein, which are each hereby incorporated by reference in their entireties, particularly with respect promoters and plasmid systems for expressing polypeptides of interest). Reported glucose isomerase promoter mutants can be used to vary the level of expression of the polypeptide encoded by a nucleic acid operably linked to the glucose isomerase promoter (U.S. Pat. No. 7,132,527). In various embodiments, the glucose isomerase promoter is contained in a low, medium, or high copy plasmid (U.S. Pat. No. 7,132,527).

In various embodiments, an isoprene synthase, DXS, IDI, and/or MVA pathway nucleic acid is contained in a low copy plasmid (e.g., a plasmid that is maintained at about 1 to about 4 copies per cell), medium copy plasmid (e.g., a plasmid that is maintained at about 10 to about 15 copies per cell), or high copy plasmid (e.g., a plasmid that is maintained at about 50 or more copies per cell). In some embodiments, the heterologous or extra endogenous isoprene synthase, DXS, IDI, or MVA pathway nucleic acid is operably linked to a T7 promoter. In some embodiments, the heterologous or extra endogenous isoprene synthase, DXS, IDI, or MVA pathway nucleic acid operably linked to a T7 promoter is contained in a medium or high copy plasmid. In some embodiments, the heterologous or extra endogenous isoprene synthase, DXS, IDI, or MVA pathway nucleic acid is operably linked to a Trc promoter. In some embodiments, the heterologous or extra endogenous isoprene synthase, DXS, IDI, or MVA pathway nucleic acid operably linked to a Trc promoter is contained in a medium or high copy plasmid. In some embodiments, the heterologous or extra endogenous isoprene synthase, DXS, IDI, or MVA pathway nucleic acid is operably linked to a Lac promoter. In some embodiments, the heterologous or extra endogenous isoprene synthase, DXS, IDI, or MVA pathway nucleic acid operably linked to a Lac promoter is contained in a low copy plasmid. In some embodiments, the heterologous or extra endogenous isoprene synthase, DXS, IDI, or MVA pathway nucleic acid is operably linked to an endogenous promoter, such as an endogenous Escherichia, Panteoa, Bacillus, Yarrowia, Streptomyces, or Trichoderma promoter or an endogenous alkaline serine protease, isoprene synthase, DXS, IDI, or MVA pathway promoter. In some embodiments, the heterologous or extra endogenous isoprene synthase, DXS, IDI, or MVA pathway nucleic acid operably linked to an endogenous promoter is contained in a high copy plasmid. In some embodiments, the vector is a replicating plasmid that does not integrate into a chromosome in the cells. In some embodiments, part or all of the vector integrates into a chromosome in the cells.

In some embodiments, the vector is any vector which when introduced into a fungal host cell is integrated into the host cell genome and is replicated. Reference is made to the Fungal Genetics Stock Center Catalogue of Strains (FGSC, the world-wide web at “fgsc.net” and the references cited therein, which are each hereby incorporated by reference in their entireties, particularly with respect to vectors) for a list of vectors. Additional examples of suitable expression and/or integration vectors are provided in Sambrook et al., Molecular Cloning: A Laboratory Manual, 2^{nd} ed., Cold Spring Harbor, 1989, Current Protocols in Molecular Biology (F. M. Ausubel et al. (eds) 1987, Supplement 30, section 7.7.18); van den Hondel et al. in Bennett and Lasure (Eds.) More Gene Manipulations in Fungi, Academic Press pp. 396-428, 1991; and U.S. Pat. No. 5,874,276, which are each hereby incorporated by reference in their entireties, particularly with respect to vectors. Particularly useful vectors include pFB6, pBR322, PUC18, pUC100, and pENTR/D.

In some embodiments, an isoprene synthase, DXS, IDI, or MVA pathway nucleic acid is operably linked to a suitable promoter that shows transcriptional activity in a fungal host cell. The promoter may be derived from one or more nucleic acids encoding a polypeptide that is either endogenous or heterologous to the host cell. In some embodiments, the promoter is useful in a Trichoderma host. Suitable non-limiting examples of promoters include cbh1, cbh2, egl1, egl2, pepA, hfb1, hfb2, xyn1, and amy. In some embodiments, the promoter is one that is native to the host cell. For example, in some embodiments when T. reesei is the host, the promoter is a native T. reesei promoter. In some embodiments, the promoter is T. reesei cbh1, which is an inducible promoter and has been deposited in GenBank under Accession No. D86235, which is incorporated by reference in its entirety, particularly with respect to promoters. In some embodiments, the promoter is one that is heterologous to the fungal host cell. Other examples of useful promoters include promoters from the genes of A. awamori and A. niger glucoamylase (glaA) (Nunberg et al., Mol. Cell Biol. 4:2306-2315, 1984 and Boel et al., EMBO J. 3:1581-1585, 1984, which are each hereby incorporated by reference in their entireties, particularly with respect to promoters); Aspergillus niger alpha amylases, Aspergillus oryzae TAKA amylase, T. reesei xln1, and the T. reesei cellobiohydrolase 1 (EP 137280, which is incorporated by reference in its entirety, particularly with respect to promoters).

In some embodiments, the expression vector also includes a termination sequence. Termination control regions may also be derived from various genes native to the host cell. In some embodiments, the termination sequence and the promoter sequence are derived from the same source. In another embodiment, the termination sequence is endogenous to the host cell. A particularly suitable terminator sequence is cbh1 derived from a Trichoderma strain (such as T. reesei). Other useful fungal terminators include the terminator from an A. niger or A. awamori glucoamylase nucleic acid (Nunberg et al., Mol. Cell Biol. 4:2306-2315, 1984 and Boel et al., EMBO J. 3:1581-1585, 1984; which are each hereby incorporated by reference in their entireties, particularly with respect to fungal terminators). Optionally, a termination site may be included. For effective expression of the polypeptides, DNA encoding the polypeptide are linked operably through initiation codons to selected expression control regions such that expression results in the formation of the appropriate messenger RNA.

In some embodiments, the promoter, coding, region, and terminator all originate from the isoprene synthase, DXS, IDI, or MVA pathway nucleic acid to be expressed. In some embodiments, the coding region for an isoprene synthase, DXS, IDI, or MVA pathway nucleic acid is inserted into a general-purpose expression vector such that it is under the transcriptional control of the expression construct promoter and terminator sequences. In some embodiments, genes or part thereof are inserted downstream of the strong cbh1 promoter.

An isoprene synthase, DXS, IDI, or MVA pathway nucleic acid can be incorporated into a vector, such as an expression vector, using standard techniques (Sambrook et al., Molecular Cloning: A Laboratory Manual, Cold Spring Harbor, 1982, which is hereby incorporated by reference in its entirety, particularly with respect to the screening of appropriate DNA sequences and the construction of vectors). Methods used to ligate the DNA construct comprising a nucleic acid of interest (such as an isoprene synthase, DXS, IDI, or MVA pathway nucleic acid), a promoter, a terminator, and other sequences and to insert them into a suitable vector are well known in the art. For example, restriction enzymes can be used to cleave the isoprene synthase, DXS, IDI, or MVA pathway nucleic acid and the vector. Then, the compatible ends of the cleaved isoprene synthase, DXS, IDI, or MVA pathway nucleic acid and the cleaved vector can be ligated. Linking is generally accomplished by ligation at convenient restriction sites. If such sites do not exist, the synthetic oligonucleotide linkers are used in accordance with conventional practice (see, Sambrook et al., Molecular Cloning: A Laboratory Manual, 2^{nd} ed., Cold Spring Harbor, 1989, and Bennett and Lasure, More Gene Manipulations in Fungi, Academic Press, San Diego, pp 70-76, 1991, which are each hereby incorporated by reference in their entireties, particularly with respect to oligonucleotide linkers). Additionally, vectors can be constructed using known recombination techniques (e.g., Invitrogen Life Technologies, Gateway Technology).

In some embodiments, it may be desirable to over-express isoprene synthase, DXS, IDI, or MVA pathway nucleic acids at levels far higher than currently found in naturally-occurring cells. This result may be accomplished by the selective cloning of the nucleic acids encoding those polypeptides into multicopy plasmids or placing those nucleic acids under a strong inducible or constitutive promoter. Methods for over-expressing desired polypeptides are common and well known in the art of molecular biology and examples may be found in Sambrook et al., Molecular Cloning: A Laboratory Manual, 2^{nd} ed., Cold Spring Harbor, 1989, which is hereby incorporated by reference in its entirety, particularly with respect to cloning techniques.

The following resources include descriptions of additional general methodology useful in accordance with the invention: Kreigler, Gene Transfer and Expression; A Laboratory Manual, 1990 and Ausubel et al., Eds. Current Protocols in Molecular Biology, 1994, which are each hereby incorporated by reference in their entireties, particularly with respect to molecular biology and cloning techniques.

### Exemplary Source Organisms

Isoprene synthase, DXS, IDI, or MVA pathway nucleic acids (and their encoded polypeptides) can be obtained from any organism that naturally contains isoprene synthase, DXS, IDI, and/or MVA pathway nucleic acids. As noted above, isoprene is formed naturally by a variety of organisms, such as bacteria, yeast, plants, and animals. Organisms contain the MVA pathway, DXP pathway, or both the MVA and DXP pathways for producing isoprene (FIGS. 19A and 19B). Thus, DXS nucleic acids can be obtained, e.g., from any organism that contains the DXP pathway or contains both the MVA and DXP pathways. IDI and isoprene synthase nucleic acids can be obtained, e.g., from any organism that contains the MVA pathway, DXP pathway, or both the MVA and DXP pathways. MVA pathway nucleic acids can be obtained, e.g., from any organism that contains the MVA pathway or contains both the MVA and DXP pathways.

In some embodiments, the nucleic acid sequence of the isoprene synthase, DXS, IDI, or MVA pathway nucleic is identical to the sequence of a nucleic acid that is produced by any of the following organisms in nature. In some embodiments, the amino acid sequence of the isoprene synthase, DXS, IDI, or MVA pathway polypeptide is identical to the sequence of a polypeptide that is produced by any of the following organisms in nature. In some embodiments, the isoprene synthase, DXS, IDI, or MVA pathway nucleic acid or polypeptide is a mutant nucleic acid or polypeptide derived from any of the organisms described herein. As used herein, “derived from” refers to the source of the nucleic acid or polypeptide into which one or more mutations is introduced. For example, a polypeptide that is “derived from a plant polypeptide” refers to polypeptide of interest that results from introducing one or more mutations into the sequence of a wild-type (i.e., a sequence occurring in nature) plant polypeptide.

In some embodiments, the source organism is a fungus, examples of which are species of Aspergillus such as A. oryzae and A. niger, species of Saccharomyces such as S. cerevisiae, species of Schizosaccharomyces such as S. pombe, and species of Trichoderma such as T. reesei. In some embodiments, the source organism is a filamentous fungal cell. The term “filamentous fungi” refers to all filamentous forms of the subdivision Eumycotina (see, Alexopoulos, C. J. (1962), Introductory Mycology, Wiley, N.Y.). These fungi are characterized by a vegetative mycelium with a cell wall composed of chitin, cellulose, and other complex polysaccharides. The filamentous fungi are morphologically, physiologically, and genetically distinct from yeasts. Vegetative growth by filamentous fungi is by hyphal elongation and carbon catabolism is obligatory aerobic. The filamentous fungal parent cell may be a cell of a species of, but not limited to, Trichoderma, (e.g., Trichoderma reesei, the asexual morph of Hypocrea jecorina, previously classified as T. longibrachiatum, Trichoderma viride, Trichoderma koningii, Trichoderma harzianum) (Sheir-Neirs et al., Appl. Microbiol. Biotechnol 20: 46-53, 1984; ATCC No. 56765 and ATCC No. 26921); Penicillium sp., Humicola sp. (e.g., H. insolens, H. lanuginose, or H. grisea); Chrysosporium sp. (e.g., C. lucknowense), Gliocladium sp., Aspergillus sp. (e.g., A. oryzae, A. niger, A sojae, A. japonicus, A. nidulans, or A. awamori) (Ward et al., Appl. Microbiol. Biotechnol. 39: 7380743, 1993 and Goedegebuur et al., Genet 41: 89-98, 2002), Fusarium sp., (e.g., F. roseum, F. graminum F. cerealis, F. oxysporuim, or F. venenatum), Neurospora sp., (e.g., N. crassa), Hypocrea sp., Mucor sp., (e.g., M. miehei), Rhizopus sp. and Emericella sp. (see also, Innis et al., Sci. 228: 21-26, 1985). The term “Trichoderma” or “Trichoderma sp.” or “Trichoderma spp.” refer to any fungal genus previously or currently classified as Trichoderma.

In some embodiments, the fungus is A. nidulans, A. awamori, A. oryzae, A. aculeatus, A. niger, A. japonicus, T. reesei, T. viride, F. oxysporum, or F. solani. Aspergillus strains are disclosed in Ward et al., Appl. Microbiol. Biotechnol. 39:738-743, 1993 and Goedegebuur et al., Curr Gene 41:89-98, 2002, which are each hereby incorporated by reference in their entireties, particularly with respect to fungi. In particular embodiments, the fungus is a strain of Trichoderma, such as a strain of T. reesei. Strains of T. reesei are known and non-limiting examples include ATCC No. 13631, ATCC No. 26921, ATCC No. 56764, ATCC No. 56765, ATCC No. 56767, and NRRL 15709, which are each hereby incorporated by reference in their entireties, particularly with respect to strains of T. reesei. In some embodiments, the host strain is a derivative of RL-P37. RL-P37 is disclosed in Sheir-Neiss et al., Appl. Microbiol. Biotechnology 20:46-53, 1984, which is hereby incorporated by reference in its entirety, particularly with respect to strains of T. reesei.

In some embodiments, the source organism is a yeast, such as Saccharomyces sp., Schizosaccharomyces sp., Pichia sp., or Candida sp.

In some embodiments, the source organism is a bacterium, such as strains of Bacillus such as B. lichenformis or B. subtilis, strains of Pantoea such as P. citrea, strains of Pseudomonas such as P. alcaligenes, strains of Streptomyces such as S. lividans or S. rubiginosus, or strains of Escherichia such as E. coli.

As used herein, “the genus Bacillus” includes all species within the genus “Bacillus,” as known to those of skill in the art, including but not limited to B. subtilis, B. lichemformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus.” The production of resistant endospores in the presence of oxygen is considered the defining feature of the genus Bacillus, although this characteristic also applies to the recently named Alicyclobacillus, Amphibacillus, Aneurinibacillus, Anoxybacillus, Brevibacillus, Filobacillus, Gracilibacillus, Halobacillus, Paenibacillus, Salibacillus, Thermobacillus, Ureibacillus, and Virgibacillus.

In some embodiments, the source organism is a gram-positive bacterium. Non-limiting examples include strains of Streptomyces (e.g., S. lividans, S. coelicolor, or S. griseus) and Bacillus. In some embodiments, the source organism is a gram-negative bacterium, such as E. coli or Pseudomonas sp.

In some embodiments, the source organism is a plant, such as a plant from the family Fabaceae, such as the Faboideae subfamily. In some embodiments, the source organism is kudzu, poplar (such as Populus alba x tremula CAC35696), aspen (such as Populus tremuloides), or Quercus robur.

In some embodiments, the source organism is an algae, such as a green algae, red algae, glaucophytes, chlorarachniophytes, euglenids, chromista, or dinoflagellates.

In some embodiments, the source organism is a cyanobacteria, such as cyanobacteria classified into any of the following groups based on morphology: Chroococcales, Pleurocapsales, Oscillatoriales, Nostocales, or Stigonematales.

In some embodiments, the source organism is an archaeon, such as Methanosarcina mazei. Exemplary archaea include those disclosed by Koga and Morii (Microbiology & Mol. Biology Reviews, 71:97-120, 2007, which is hereby incorporated by reference in its entirety, particularly with respect to archaea (see Table 3)). Other exemplary archaea are hyperthermophilic archaea, such as Methanococcus jannaschii (Huang et al., Protein Expression and Purification 17(1):33-40, 1999) and halophilic archaea (such as Halobacterium salanarium).

TABLE 3
Exemplary archaea

| | Exemplary | |
|---|---|---|
| Original name | Strain | Name most recently proposed |
| Caldariella acidophila | | Sulfolobus solfataricus |
| Halobacterium cutirubrum | | Halobacterium salinarum |
| Halobacterium halobium | | Halobacterium salinarum |
| Halobacterium mediterranei | | Haloferax mediterranei |
| Halobacterium vallismortis | | Haloarcula vallismortis |
| Methanobacterium | ΔH | Methanothermobacter |
| thermoautotrophicum | | thermautotrophicus |
| Methanobacterium | Marburg | Methanothermobacter |
| thermoautotrophicum | | marburgensis |
| Methanobacterium | SF-4 | Methanothermobacter wolfeii |
| thermoformicicum | | |
| Methanococcus igneus | | Methanotorris igneus |
| Natronobacterium pharaonis | | Natronomonas pharaonis |
| Pseudomonas salinaria | | Halobacterium salinarum |

### Exemplary Host Cells

A variety of host cells can be used to express isoprene synthase, DXS, IDI, and/or MVA pathway polypeptides and to produce isoprene in the methods of the invention. Exemplary host cells include cells from any of the organisms listed in the prior section under the heading “Exemplary Source Organisms.” The host cell may be a cell that naturally produces isoprene or a cell that does not naturally produce isoprene. In some embodiments, the host cell naturally produces isoprene using the DXP pathway, and an isoprene synthase, DXS, and/or IDI nucleic acid is added to enhance production of isoprene using this pathway. In some embodiments, the host cell naturally produces isoprene using the MVA pathway, and an isoprene synthase and/or one or more MVA pathway nucleic acids are added to enhance production of isoprene using this pathway. In some embodiments, the host cell naturally produces isoprene using the DXP pathway and one or more MVA pathway nucleic acids are added to produce isoprene using part or all of the MVA pathway as well as the DXP pathway. In some embodiments, the host cell naturally produces isoprene using both the DXP and MVA pathways and one or more isoprene synthase, DXS, IDI, or MVA pathway nucleic acids are added to enhance production of isoprene by one or both of these pathways.

### Exemplary Transformation Methods

Isoprene synthase, DXS, IDI, and/or MVA pathway nucleic acids or vectors containing them can be inserted into a host cell (e.g., a plant cell, a fungal cell, a yeast cell, or a bacterial cell described herein) using standard techniques for expression of the encoded isoprene synthase, DXS, IDI, and/or MVA pathway polypeptide. Introduction of a DNA construct or vector into a host cell can be performed using techniques such as transformation, electroporation, nuclear microinjection, transduction, transfection (e.g., lipofection mediated or DEAE-Dextrin mediated transfection or transfection using a recombinant phage virus), incubation with calcium phosphate DNA precipitate, high velocity bombardment with DNA-coated microprojectiles, and protoplast fusion. General transformation techniques are known in the art (see, e.g., Current Protocols in Molecular Biology (F. M. Ausubel et al. (eds) Chapter 9, 1987; Sambrook et al., Molecular Cloning: A Laboratory Manual, 2^{nd} ed., Cold Spring Harbor, 1989; and Campbell et al., Curr. Genet. 16:53-56, 1989, which are each hereby incorporated by reference in their entireties, particularly with respect to transformation methods). The expression of heterologous polypeptide in Trichoderma is described in U.S. Pat. No. 6,022,725; U.S. Pat. No. 6,268,328; U.S. Pat. No. 7,262,041; WO 2005/001036; Harkki et al.; Enzyme Microb. Technol. 13:227-233, 1991; Harkki et al., Bio Technol. 7:596-603, 1989; EP 244,234; EP 215,594; and Nevalainen et al., “The Molecular Biology of Trichoderma and its Application to the Expression of Both Homologous and Heterologous Genes,” in Molecular Industrial Mycology, Eds. Leong and Berka, Marcel Dekker Inc., NY pp. 129-148, 1992, which are each hereby incorporated by reference in their entireties, particularly with respect to transformation and expression methods). Reference is also made to Cao et al., (Sci. 9:991-1001, 2000; EP 238023; and Yelton et al., Proceedings. Natl. Acad. Sci. USA 81:1470-1474, 1984 (which are each hereby incorporated by reference in their entireties, particularly with respect to transformation methods) for transformation of Aspergillus strains. The introduced nucleic acids may be integrated into chromosomal DNA or maintained as extrachromosomal replicating sequences.

Any method known in the art may be used to select transformants. In one non-limiting example, stable transformants including an amdS marker are distinguished from unstable transformants by their faster growth rate and the formation of circular colonies with a smooth, rather than ragged outline on solid culture medium containing acetamide. Additionally, in some cases a further test of stability is conducted by growing the transformants on a solid non-selective medium (e.g., a medium that lacks acetamide), harvesting spores from this culture medium, and determining the percentage of these spores which subsequently germinate and grow on selective medium containing acetamide.

In some embodiments, fungal cells are transformed by a process involving protoplast formation and transformation of the protoplasts followed by regeneration of the cell wall in a known manner. In one specific embodiment, the preparation of Trichoderma sp. for transformation involves the preparation of protoplasts from fungal mycelia (see, Campbell et al., Curr. Genet. 16:53-56, 1989, which is incorporated by reference in its entirety, particularly with respect to transformation methods). In some embodiments, the mycelia are obtained from germinated vegetative spores. The mycelia are treated with an enzyme that digests the cell wall resulting in protoplasts. The protoplasts are then protected by the presence of an osmotic stabilizer in the suspending medium. These stabilizers include sorbitol, mannitol, potassium chloride, magnesium sulfate, and the like. Usually the concentration of these stabilizers varies between 0.8 M and 1.2 M. It is desirable to use about a 1.2 M solution of sorbitol in the suspension medium.

Uptake of DNA into the host Trichoderma sp. strain is dependent upon the calcium ion concentration. Generally, between about 10 mM CaCl₂ and 50 mM CaCl₂ is used in an uptake solution. In addition to the calcium ion in the uptake solution, other compounds generally included are a buffering system such as TE buffer (10 Mm Tris, pH 7.4; 1 mM EDTA) or 10 mM MOPS, pH 6.0 buffer (morpholinepropanesulfonic acid) and polyethylene glycol (PEG). While not intending to be bound to any particular theory, it is believed that the polyethylene glycol acts to fuse the cell membranes, thus permitting the contents of the medium to be delivered into the cytoplasm of the Trichoderma sp. strain and the plasmid DNA to be transferred to the nucleus. This fusion frequently leaves multiple copies of the plasmid DNA integrated into the host chromosome.

Usually a suspension containing the Trichoderma sp. protoplasts or cells that have been subjected to a permeability treatment at a density of 10⁵ to 10⁷/mL (such as 2×10⁶/mL) are used in the transformation. A volume of 100 μL of these protoplasts or cells in an appropriate solution (e.g., 1.2 M sorbitol and 50 mM CaCl₂) are mixed with the desired DNA. Generally, a high concentration of PEG is added to the uptake solution. From 0.1 to 1 volume of 25% PEG 4000 can be added to the protoplast suspension. In some embodiments, about 0.25 volumes are added to the protoplast suspension. Additives such as dimethyl sulfoxide, heparin, spermidine, potassium chloride, and the like may also be added to the uptake solution and aid in transformation. Similar procedures are available for other fungal host cells (see, e.g., U.S. Pat. Nos. 6,022,725 and 6,268,328, which are each hereby incorporated by reference in their entireties, particularly with respect to transformation methods).

Generally, the mixture is then cultured at approximately 0° C. for a period of between 10 to 30 minutes. Additional PEG is then added to the mixture to further enhance the uptake of the desired nucleic acid sequence. The 25% PEG 4000 is generally added in volumes of 5 to 15 times the volume of the transformation mixture; however, greater and lesser volumes may be suitable. The 25% PEG 4000 is desirably about 10 times the volume of the transformation mixture. After the PEG is added, the transformation mixture is then cultured either at room temperature or on ice before the addition of a sorbitol and CaCl₂ solution. The protoplast suspension is then further added to molten aliquots of a growth medium. When the growth medium includes a growth selection (e.g., acetamide or an antibiotic) it permits the growth of transformants only.

The transformation of bacterial cells may be performed according to conventional methods, e.g., as described in Sambrook et al., Molecular Cloning: A Laboratory Manual, Cold Spring Harbor, 1982, which is hereby incorporated by reference in its entirety, particularly with respect to transformation methods.

### Exemplary Cell Culture Media

The invention also includes a cell or a population of cells in culture that produce isoprene. By “cells in culture” is meant two or more cells in a solution (e.g., a cell medium) that allows the cells to undergo one or more cell divisions. “Cells in culture” do not include plant cells that are part of a living, multicellular plant containing cells that have differentiated into plant tissues. In various embodiments, the cell culture includes at least or about 10, 20, 50, 100, 200, 500, 1,000, 5,000, 10,000 or more cells.

Any carbon source can be used to cultivate the host cells. The term “carbon source” refers to one or more carbon-containing compounds capable of being metabolized by a host cell or organism. For example, the cell medium used to cultivate the host cells may include any carbon source suitable for maintaining the viability or growing the host cells.

In some embodiments, the carbon source is a carbohydrate (such as monosaccharide, disaccharide, oligosaccharide, or polysaccharide), invert sugar (e.g., enzymatically treated sucrose syrup), glycerol, glycerine (e.g., a glycerine byproduct of a biodiesel or soap-making process), dihydroxyacetone, one-carbon source, oil (e.g., a plant or vegetable oil such as corn, palm, or soybean oil), animal fat, animal oil, fatty acid (e.g., a saturated fatty acid, unsaturated fatty acid, or polyunsaturated fatty acid), lipid, phospholipid, glycerolipid, monoglyceride, diglyceride, triglyceride, polypeptide (e.g., a microbial or plant protein or peptide), renewable carbon source (e.g., a biomass carbon source such as a hydrolyzed biomass carbon source), yeast extract, component from a yeast extract, polymer, acid, alcohol, aldehyde, ketone, amino acid, succinate, lactate, acetate, ethanol, or any combination of two or more of the foregoing. In some embodiments, the carbon source is a product of photosynthesis, including, but not limited to, glucose.

Exemplary monosaccharides include glucose and fructose; exemplary oligosaccharides include lactose and sucrose, and exemplary polysaccharides include starch and cellulose. Exemplary carbohydrates include C6 sugars (e.g., fructose, mannose, galactose, or glucose) and C5 sugars (e.g., xylose or arabinose). In some embodiments, the cell medium includes a carbohydrate as well as a carbon source other than a carbohydrate (e.g., glycerol, glycerine, dihydroxyacetone, one-carbon source, oil, animal fat, animal oil, fatty acid, lipid, phospholipid, glycerolipid, monoglyceride, diglyceride, triglyceride, renewable carbon source, or a component from a yeast extract). In some embodiments, the cell medium includes a carbohydrate as well as a polypeptide (e.g., a microbial or plant protein or peptide). In some embodiments, the microbial polypeptide is a polypeptide from yeast or bacteria. In some embodiments, the plant polypeptide is a polypeptide from soy, corn, canola, jatropha, palm, peanut, sunflower, coconut, mustard, rapeseed, cottonseed, palm kernel, olive, safflower, sesame, or linseed.

In some embodiments, the concentration of the carbohydrate is at least or about 5 grams per liter of broth (g/L, wherein the volume of broth includes both the volume of the cell medium and the volume of the cells), such as at least or about 10, 15, 20, 30, 40, 50, 60, 80, 100, 150, 200, 300, 400, or more g/L. In some embodiments, the concentration of the carbohydrate is between about 50 and about 400 g/L, such as between about 100 and about 360 g/L, between about 120 and about 360 g/L, or between about 200 and about 300 g/L. In some embodiments, this concentration of carbohydrate includes the total amount of carbohydrate that is added before and/or during the culturing of the host cells.

In some embodiments, the cells are cultured under limited glucose conditions. By “limited glucose conditions” is meant that the amount of glucose that is added is less than or about 105% (such as about 100%) of the amount of glucose that is consumed by the cells. In particular embodiments, the amount of glucose that is added to the culture medium is approximately the same as the amount of glucose that is consumed by the cells during a specific period of time. In some embodiments, the rate of cell growth is controlled by limiting the amount of added glucose such that the cells grow at the rate that can be supported by the amount of glucose in the cell medium. In some embodiments, glucose does not accumulate during the time the cells are cultured. In various embodiments, the cells are cultured under limited glucose conditions for greater than or about 1, 2, 3, 5, 10, 15, 20, 25, 30, 35, 40, 50, 60, or 70 hours. In various embodiments, the cells are cultured under limited glucose conditions for greater than or about 5, 10, 15, 20, 25, 30, 35, 40, 50, 60, 70, 80, 90, 95, or 100% of the total length of time the cells are cultured. While not intending to be bound by any particular theory, it is believed that limited glucose conditions may allow more favorable regulation of the cells.

In some embodiments, the cells are cultured in the presence of an excess of glucose. In particular embodiments, the amount of glucose that is added is greater than about 105% (such as about or greater than 110, 120, 150, 175, 200, 250, 300, 400, or 500%) or more of the amount of glucose that is consumed by the cells during a specific period of time. In some embodiments, glucose accumulates during the time the cells are cultured.

Exemplary lipids are any substance containing one or more fatty acids that are C4 and above fatty acids that are saturated, unsaturated, or branched.

Exemplary oils are lipids that are liquid at room temperature. In some embodiments, the lipid contains one or more C4 or above fatty acids (e.g., contains one or more saturated, unsaturated, or branched fatty acid with four or more carbons). In some embodiments, the oil is obtained from soy, corn, canola, jatropha, palm, peanut, sunflower, coconut, mustard, rapeseed, cottonseed, palm kernel, olive, safflower, sesame, linseed, oleagineous microbial cells, Chinese tallow, or any combination of two or more of the foregoing.

Exemplary fatty acids include compounds of the formula RCOOH, where “R” is a hydrocarbon. Exemplary unsaturated fatty acids include compounds where “R” includes at least one carbon-carbon double bond. Exemplary unsaturated fatty acids include, but are not limited to, oleic acid, vaccenic acid, linoleic acid, palmitelaidic acid, and arachidonic acid. Exemplary polyunsaturated fatty acids include compounds where “R” includes a plurality of carbon-carbon double bonds. Exemplary saturated fatty acids include compounds where “R” is a saturated aliphatic group. In some embodiments, the carbon source includes one or more C₁₂-C₂₂ fatty acids, such as a C₁₂ saturated fatty acid, a C₁₄ saturated fatty acid, a C₁₆ saturated fatty acid, a C₁₈ saturated fatty acid, a C₂₀ saturated fatty acid, or a C₂₂ saturated fatty acid. In an exemplary embodiment, the fatty acid is palmitic acid. In some embodiments, the carbon source is a salt of a fatty acid (e.g., an unsaturated fatty acid), a derivative of a fatty acid (e.g., an unsaturated fatty acid), or a salt of a derivative of fatty acid (e.g., an unsaturated fatty acid). Suitable salts include, but are not limited to, lithium salts, potassium salts, sodium salts, and the like. Di- and triglycerols are fatty acid esters of glycerol.

In some embodiments, the concentration of the lipid, oil, fat, fatty acid, monoglyceride, diglyceride, or triglyceride is at least or about 1 gram per liter of broth (g/L, wherein the volume of broth includes both the volume of the cell medium and the volume of the cells), such as at least or about 5, 10, 15, 20, 30, 40, 50, 60, 80, 100, 150, 200, 300, 400, or more g/L. In some embodiments, the concentration of the lipid, oil, fat, fatty acid, monoglyceride, diglyceride, or triglyceride is between about 10 and about 400 g/L, such as between about 25 and about 300 g/L, between about 60 and about 180 g/L, or between about 75 and about 150 g/L. In some embodiments, the concentration includes the total amount of the lipid, oil, fat, fatty acid, monoglyceride, diglyceride, or triglyceride that is added before and/or during the culturing of the host cells. In some embodiments, the carbon source includes both (i) a lipid, oil, fat, fatty acid, monoglyceride, diglyceride, or triglyceride and (ii) a carbohydrate, such as glucose. In some embodiments, the ratio of the lipid, oil, fat, fatty acid, monoglyceride, diglyceride, or triglyceride to the carbohydrate is about 1:1 on a carbon basis (i.e., one carbon in the lipid, oil, fat, fatty acid, monoglyceride, diglyceride, or triglyceride per carbohydrate carbon). In particular embodiments, the amount of the lipid, oil, fat, fatty acid, monoglyceride, diglyceride, or triglyceride is between about 60 and 180 g/L, and the amount of the carbohydrate is between about 120 and 360 g/L.

Exemplary microbial polypeptide carbon sources include one or more polypeptides from yeast or bacteria. Exemplary plant polypeptide carbon sources include one or more polypeptides from soy, corn, canola, jatropha, palm, peanut, sunflower, coconut, mustard, rapeseed, cottonseed, palm kernel, olive, safflower, sesame, or linseed.

Exemplary renewable carbon sources include cheese whey permeate, cornsteep liquor, sugar beet molasses, barley malt, and components from any of the foregoing. Exemplary renewable carbon sources also include glucose, hexose, pentose and xylose present in biomass, such as corn, switchgrass, sugar cane, cell waste of fermentation processes, and protein by-product from the milling of soy, corn, or wheat. In some embodiments, the biomass carbon source is a lignocellulosic, hemicellulosic, or cellulosic material such as, but are not limited to, a grass, wheat, wheat straw, bagasse, sugar cane bagasse, soft wood pulp, corn, corn cob or husk, corn kernel, fiber from corn kernels, corn stover, switch grass, rice hull product, or a by-product from wet or dry milling of grains (e.g., corn, sorghum, rye, triticate, barley, wheat, and/or distillers grains). Exemplary cellulosic materials include wood, paper and pulp waste, herbaceous plants, and fruit pulp. In some embodiments, the carbon source includes any plant part, such as stems, grains, roots, or tubers. In some embodiments, all or part of any of the following plants are used as a carbon source: corn, wheat, rye, sorghum, triticate, rice, millet, barley, cassava, legumes, such as beans and peas, potatoes, sweet potatoes, bananas, sugarcane, and/or tapioca. In some embodiments, the carbon source is a biomass hydrolysate, such as a biomass hydrolysate that includes both xylose and glucose or that includes both sucrose and glucose.

In some embodiments, the renewable carbon source (such as biomass) is pretreated before it is added to the cell culture medium. In some embodiments, the pretreatment includes enzymatic pretreatment, chemical pretreatment, or a combination of both enzymatic and chemical pretreatment (see, for example, Farzaneh et al., Bioresource Technology 96 (18): 2014-2018, 2005; U.S. Pat. No. 6,176,176; U.S. Pat. No. 6,106,888; which are each hereby incorporated by reference in their entireties, particularly with respect to the pretreatment of renewable carbon sources). In some embodiments, the renewable carbon source is partially or completely hydrolyzed before it is added to the cell culture medium.

In some embodiments, the renewable carbon source (such as corn stover) undergoes ammonia fiber expansion (AFEX) pretreatment before it is added to the cell culture medium (see, for example, Farzaneh et al., Bioresource Technology 96 (18): 2014-2018, 2005). During AFEX pretreatment, a renewable carbon source is treated with liquid anhydrous ammonia at moderate temperatures (such as about 60 to about 100° C.) and high pressure (such as about 250 to about 300 psi) for about 5 minutes. Then, the pressure is rapidly released. In this process, the combined chemical and physical effects of lignin solubilization, hemicellulose hydrolysis, cellulose decrystallization, and increased surface area enables near complete enzymatic conversion of cellulose and hemicellulose to fermentable sugars. AFEX pretreatment has the advantage that nearly all of the ammonia can be recovered and reused, while the remaining serves as nitrogen source for microbes in downstream processes. Also, a wash stream is not required for AFEX pretreatment. Thus, dry matter recovery following the AFEX treatment is essentially 100%. AFEX is basically a dry to dry process. The treated renewable carbon source is stable for long periods and can be fed at very high solid loadings in enzymatic hydrolysis or fermentation processes. Cellulose and hemicellulose are well preserved in the AFEX process, with little or no degradation. There is no need for neutralization prior to the enzymatic hydrolysis of a renewable carbon source that has undergone AFEX pretreatment. Enzymatic hydrolysis of AFEX-treated carbon sources produces clean sugar streams for subsequent fermentation use.

In some embodiments, the concentration of the carbon source (e.g., a renewable carbon source) is equivalent to at least or about 0.1, 0.5, 1, 1.5 2, 3, 4, 5, 10, 15, 20, 30, 40, or 50% glucose (w/v). The equivalent amount of glucose can be determined by using standard HPLC methods with glucose as a reference to measure the amount of glucose generated from the carbon source. In some embodiments, the concentration of the carbon source (e.g., a renewable carbon source) is equivalent to between about 0.1 and about 20% glucose, such as between about 0.1 and about 10% glucose, between about 0.5 and about 10% glucose, between about 1 and about 10% glucose, between about 1 and about 5% glucose, or between about 1 and about 2% glucose.

In some embodiments, the carbon source includes yeast extract or one or more components of yeast extract. In some embodiments, the concentration of yeast extract is at least 1 gram of yeast extract per liter of broth (g/L, wherein the volume of broth includes both the volume of the cell medium and the volume of the cells), such at least or about 5, 10, 15, 20, 30, 40, 50, 60, 80, 100, 150, 200, 300, or more g/L. In some embodiments, the concentration of yeast extract is between about 1 and about 300 g/L, such as between about 1 and about 200 g/L, between about 5 and about 200 g/L, between about 5 and about 100 g/L, or between about 5 and about 60 g/L. In some embodiments, the concentration includes the total amount of yeast extract that is added before and/or during the culturing of the host cells. In some embodiments, the carbon source includes both yeast extract (or one or more components thereof) and another carbon source, such as glucose. In some embodiments, the ratio of yeast extract to the other carbon source is about 1:5, about 1:10, or about 1:20 (w/w).

Additionally the carbon source may also be one-carbon substrates such as carbon dioxide, or methanol. Glycerol production from single carbon sources (e.g., methanol, formaldehyde, or formate) has been reported in methylotrophic yeasts (Yamada et al., Agric. Biol. Chem., 53(2) 541-543, 1989, which is hereby incorporated by reference in its entirety, particularly with respect to carbon sources) and in bacteria (Hunter et. al., Biochemistry, 24, 4148-4155, 1985, which is hereby incorporated by reference in its entirety, particularly with respect to carbon sources). These organisms can assimilate single carbon compounds, ranging in oxidation state from methane to formate, and produce glycerol. The pathway of carbon assimilation can be through ribulose monophosphate, through serine, or through xylulose-momophosphate (Gottschalk, Bacterial Metabolism, Second Edition, Springer-Verlag: New York, 1986, which is hereby incorporated by reference in its entirety, particularly with respect to carbon sources). The ribulose monophosphate pathway involves the condensation of formate with ribulose-5-phosphate to form a six carbon sugar that becomes fructose and eventually the three carbon product glyceraldehyde-3-phosphate. Likewise, the serine pathway assimilates the one-carbon compound into the glycolytic pathway via methylenetetrahydrofolate.

In addition to one and two carbon substrates, methylotrophic organisms are also known to utilize a number of other carbon containing compounds such as methylamine, glucosamine and a variety of amino acids for metabolic activity. For example, methylotrophic yeast are known to utilize the carbon from methylamine to form trehalose or glycerol (Bellion et al., Microb. Growth Cl Compd., [Int. Symp.], 7^{th} ed., 415-32. Editors: Murrell et al., Publisher: Intercept, Andover, UK, 1993, which is hereby incorporated by reference in its entirety, particularly with respect to carbon sources). Similarly, various species of Candida metabolize alanine or oleic acid (Sulter et al., Arch. Microbiol. 153(5), 485-9, 1990, which is hereby incorporated by reference in its entirety, particularly with respect to carbon sources).

In some embodiments, cells are cultured in a standard medium containing physiological salts and nutrients (see, e.g., Pourquie, J. et al., Biochemistry and Genetics of Cellulose Degradation, eds. Aubert et al., Academic Press, pp. 71-86, 1988 and Ilmen et al., Appl. Environ. Microbiol. 63:1298-1306, 1997, which are each hereby incorporated by reference in their entireties, particularly with respect to cell medias). Exemplary growth media are common commercially prepared media such as Luria Bertani (LB) broth, Sabouraud Dextrose (SD) broth, or Yeast medium (YM) broth. Other defined or synthetic growth media may also be used, and the appropriate medium for growth of particular host cells are known by someone skilled in the art of microbiology or fermentation science.

In addition to an appropriate carbon source, the cell medium desirably contains suitable minerals, salts, cofactors, buffers, and other components known to those skilled in the art suitable for the growth of the cultures or the enhancement of isoprene production (see, for example, WO 2004/033646 and references cited therein and WO 96/35796 and references cited therein, which are each hereby incorporated by reference in their entireties, particularly with respect cell medias and cell culture conditions). In some embodiments where an isoprene synthase, DXS, IDI, and/or MVA pathway nucleic acid is under the control of an inducible promoter, the inducing agent (e.g., a sugar, metal salt or antimicrobial), is desirably added to the medium at a concentration effective to induce expression of an isoprene synthase, DXS, IDI, and/or MVA pathway polypeptide. In some embodiments, cell medium has an antibiotic (such as kanamycin) that corresponds to the antibiotic resistance nucleic acid (such as a kanamycin resistance nucleic acid) on a vector that has one or more DXS, IDI, or MVA pathway nucleic acids.

### Exemplary Cell Culture Conditions

Materials and methods suitable for the maintenance and growth of bacterial cultures are well known in the art. Exemplary techniques may be found in Manual of Methods for General Bacteriology Gerhardt et al., eds), American Society for Microbiology, Washington, D.C. (1994) or Brock in Biotechnology: A Textbook of Industrial Microbiology, Second Edition (1989) Sinauer Associates, Inc., Sunderland, Mass., which are each hereby incorporated by reference in their entireties, particularly with respect to cell culture techniques. In some embodiments, the cells are cultured in a culture medium under conditions permitting the expression of one or more isoprene synthase, DXS, IDI, or MVA pathway polypeptides encoded by a nucleic acid inserted into the host cells.

Standard cell culture conditions can be used to culture the cells (see, for example, WO 2004/033646 and references cited therein, which are each hereby incorporated by reference in their entireties, particularly with respect to cell culture and fermentation conditions). Cells are grown and maintained at an appropriate temperature, gas mixture, and pH (such as at about 20 to about 37° C., at about 6% to about 84% CO₂, and at a pH between about 5 to about 9). In some embodiments, cells are grown at 35° C. in an appropriate cell medium. In some embodiments, e.g., cultures are cultured at approximately 28° C. in appropriate medium in shake cultures or fermentors until desired amount of isoprene production is achieved. In some embodiments, the pH ranges for fermentation are between about pH 5.0 to about pH 9.0 (such as about pH 6.0 to about pH 8.0 or about 6.5 to about 7.0). Reactions may be performed under aerobic, anoxic, or anaerobic conditions based on the requirements of the host cells. Exemplary culture conditions for a given filamentous fungus are known in the art and may be found in the scientific literature and/or from the source of the fungi such as the American Type Culture Collection and Fungal Genetics Stock Center.

In various embodiments, the cells are grown using any known mode of fermentation, such as batch, fed-batch, or continuous processes. In some embodiments, a batch method of fermentation is used. Classical batch fermentation is a closed system where the composition of the media is set at the beginning of the fermentation and is not subject to artificial alterations during the fermentation. Thus, at the beginning of the fermentation the cell medium is inoculated with the desired host cells and fermentation is permitted to occur adding nothing to the system. Typically, however, “batch” fermentation is batch with respect to the addition of carbon source and attempts are often made at controlling factors such as pH and oxygen concentration. In batch systems, the metabolite and biomass compositions of the system change constantly until the time the fermentation is stopped. Within batch cultures, cells moderate through a static lag phase to a high growth log phase and finally to a stationary phase where growth rate is diminished or halted. In some embodiments, cells in log phase are responsible for the bulk of the isoprene production. In some embodiments, cells in stationary phase produce isoprene.

In some embodiments, a variation on the standard batch system is used, such as the Fed-Batch system. Fed-Batch fermentation processes comprise a typical batch system with the exception that the carbon source is added in increments as the fermentation progresses. Fed-Batch systems are useful when catabolite repression is apt to inhibit the metabolism of the cells and where it is desirable to have limited amounts of carbon source in the cell medium. Fed-batch fermentations may be performed with the carbon source (e.g., glucose) in a limited or excess amount. Measurement of the actual carbon source concentration in Fed-Batch systems is difficult and is therefore estimated on the basis of the changes of measurable factors such as pH, dissolved oxygen, and the partial pressure of waste gases such as CO₂. Batch and Fed-Batch fermentations are common and well known in the art and examples may be found in Brock, Biotechnology: A Textbook of Industrial Microbiology, Second Edition (1989) Sinauer Associates, Inc., which is hereby incorporated by reference in its entirety, particularly with respect to cell culture and fermentation conditions.

In some embodiments, continuous fermentation methods are used. Continuous fermentation is an open system where a defined fermentation medium is added continuously to a bioreactor and an equal amount of conditioned medium is removed simultaneously for processing. Continuous fermentation generally maintains the cultures at a constant high density where cells are primarily in log phase growth.

Continuous fermentation allows for the modulation of one factor or any number of factors that affect cell growth or isoprene production. For example, one method maintains a limiting nutrient such as the carbon source or nitrogen level at a fixed rate and allows all other parameters to moderate. In other systems, a number of factors affecting growth can be altered continuously while the cell concentration (e.g., the concentration measured by media turbidity) is kept constant. Continuous systems strive to maintain steady state growth conditions. Thus, the cell loss due to media being drawn off is balanced against the cell growth rate in the fermentation. Methods of modulating nutrients and growth factors for continuous fermentation processes as well as techniques for maximizing the rate of product formation are well known in the art of industrial microbiology and a variety of methods are detailed by Brock, Biotechnology: A Textbook of Industrial Microbiology, Second Edition (1989) Sinauer Associates, Inc., which is hereby incorporated by reference in its entirety, particularly with respect to cell culture and fermentation conditions.

In some embodiments, cells are immobilized on a substrate as whole cell catalysts and subjected to fermentation conditions for isoprene production.

In some embodiments, bottles of liquid culture are placed in shakers in order to introduce oxygen to the liquid and maintain the uniformity of the culture. In some embodiments, an incubator is used to control the temperature, humidity, shake speed, and/or other conditions in which a culture is grown. The simplest incubators are insulated boxes with an adjustable heater, typically going up to ˜65° C. More elaborate incubators can also include the ability to lower the temperature (via refrigeration), or the ability to control humidity or CO₂ levels. Most incubators include a timer; some can also be programmed to cycle through different temperatures, humidity levels, etc. Incubators can vary in size from tabletop to units the size of small rooms.

If desired, a portion or all of the cell medium can be changed to replenish nutrients and/or avoid the build up of potentially harmful metabolic byproducts and dead cells. In the case of suspension cultures, cells can be separated from the media by centrifuging or filtering the suspension culture and then resuspending the cells in fresh media. In the case of adherent cultures, the media can be removed directly by aspiration and replaced. In some embodiments, the cell medium allows at least a portion of the cells to divide for at least or about 5, 10, 20, 40, 50, 60, 65, or more cell divisions in a continuous culture (such as a continuous culture without dilution).

In some embodiments, a constitutive or leaky promoter (such as a Trc promoter) is used and a compound (such as IPTG) is not added to induce expression of the isoprene synthase, DXS, IDI, or MVA pathway nucleic acid(s) operably linked to the promoter. In some embodiments, a compound (such as IPTG) is added to induce expression of the isoprene synthase, DXS, IDI, or MVA pathway nucleic acid(s) operably linked to the promoter.

Exemplary Methods for Decoupling Isoprene Production from Cell Growth

Desirably, carbon from the feedstock is converted to isoprene rather than to the growth and maintenance of the cells. In some embodiments, the cells are grown to a low to medium OD₆₀₀, then production of isoprene is started or increased. This strategy permits a large portion of the carbon to be converted to isoprene.

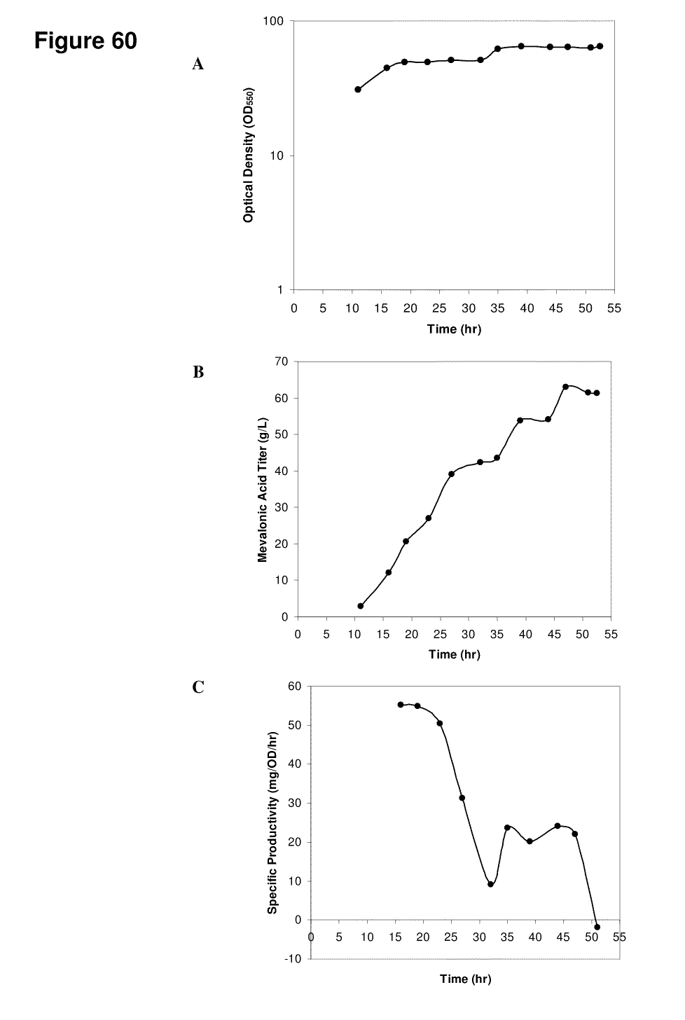

In some embodiments, cells reach an optical density such that they no longer divide or divide extremely slowly, but continue to make isoprene for several hours (such as about 2, 4, 6, 8, 10, 15, 20, 25, 30, or more hours). For example, FIGS. 60A-67C illustrate that cells may continue to produce a substantial amount of mevalonic acid or isoprene after the cells reach an optical density such that they no longer divide or divide extremely slowly. In some cases, the optical density at 550 nm decreases over time (such as a decrease in the optical density after the cells are no longer in an exponential growth phase due to cell lysis), and the cells continue to produce a substantial amount of mevalonic acid or isoprene. In some embodiments, the optical density at 550 nm of the cells increases by less than or about 50% (such as by less than or about 40, 30, 20, 10, 5, or 0%) over a certain time period (such as greater than or about 5, 10, 15, 20, 25, 30, 40, 50 or 60 hours), and the cells produce isoprene at greater than or about 1, 10, 25, 50, 100, 150, 200, 250, 300, 400, 500, 600, 700, 800, 900, 1,000, 1,250, 1,500, 1,750, 2,000, 2,500, 3,000, 4,000, 5,000, or more nmole of isoprene/gram of cells for the wet weight of the cells/hour (nmole/g_{wcm}/hr) during this time period. In some embodiments, the amount of isoprene is between about 2 to about 5,000 nmole/g_{wcm}/hr, such as between about 2 to about 100 nmole/g_{wcm}/hr, about 100 to about 500 nmole/g_{wcm}/hr, about 150 to about 500 nmole/g_{wcm}/hr, about 500 to about 1,000 nmole/g_{wcm}/hr, about 1,000 to about 2,000 nmole/g_{wcm}/hr, or about 2,000 to about 5,000 nmole/g_{wcm}/hr. In some embodiments, the amount of isoprene is between about 20 to about 5,000 nmole/g_{wcm}/hr, about 100 to about 5,000 nmole/g_{wcm}/hr, about 200 to about 2,000 nmole/g_{wcm}/hr, about 200 to about 1,000 nmole/g_{wcm}/hr, about 300 to about 1,000 nmole/g_{wcm}/hr, or about 400 to about 1,000 nmole/g_{wcm}/hr.

In some embodiments, the optical density at 550 nm of the cells increases by less than or about 50% (such as by less than or about 40, 30, 20, 10, 5, or 0%) over a certain time period (such as greater than or about 5, 10, 15, 20, 25, 30, 40, 50 or 60 hours), and the cells produce a cumulative titer (total amount) of isoprene at greater than or about 1, 10, 25, 50, 100, 150, 200, 250, 300, 400, 500, 600, 700, 800, 900, 1,000, 1,250, 1,500, 1,750, 2,000, 2,500, 3,000, 4,000, 5,000, 10,000, 50,000, 100,000, or more mg of isoprene/L of broth (mg/L_{broth}, wherein the volume of broth includes the volume of the cells and the cell medium) during this time period. In some embodiments, the amount of isoprene is between about 2 to about 5,000 mg/L_{broth}, such as between about 2 to about 100 mg/L_{broth}, about 100 to about 500 mg/L_{broth}, about 500 to about 1,000 mg/L_{broth}, about 1,000 to about 2,000 mg/L_{broth}, or about 2,000 to about 5,000 mg/L_{broth}. In some embodiments, the amount of isoprene is between about 20 to about 5,000 mg/L_{broth}, about 100 to about 5,000 mg/L_{broth}, about 200 to about 2,000 mg/L_{broth}, about 200 to about 1,000 mg/L_{broth}, about 300 to about 1,000 mg/L_{broth}, or about 400 to about 1,000 mg/L_{broth}.

In some embodiments, the optical density at 550 nm of the cells increases by less than or about 50% (such as by less than or about 40, 30, 20, 10, 5, or 0%) over a certain time period (such as greater than or about 5, 10, 15, 20, 25, 30, 40, 50 or 60 hours), and the cells convert greater than or about 0.0015, 0.002, 0.005, 0.01, 0.02, 0.05, 0.1, 0.12, 0.14, 0.16, 0.2, 0.3, 0.4, 0.5, 0.6, 0.7, 0.8, 0.9, 1.0, 1.2, 1.4, 1.6, 1.8, 2.0, 2.5, 3.0, 3.5, 4.0, 5.0, 6.0, 7.0, or 8.0% of the carbon in the cell culture medium into isoprene during this time period. In some embodiments, the percent conversion of carbon into isoprene is between such as about 0.002 to about 4.0%, about 0.002 to about 3.0%, about 0.002 to about 2.0%, about 0.002 to about 1.6%, about 0.002 to about 0.005%, about 0.005 to about 0.01%, about 0.01 to about 0.05%, about 0.05 to about 0.15%, 0.15 to about 0.2%, about 0.2 to about 0.3%, about 0.3 to about 0.5%, about 0.5 to about 0.8%, about 0.8 to about 1.0%, or about 1.0 to about 1.6%. In some embodiments, the percent conversion of carbon into isoprene is between about 0.002 to about 0.4%, 0.002 to about 0.16%, 0.04 to about 0.16%, about 0.005 to about 0.3%, about 0.01 to about 0.3%, or about 0.05 to about 0.3%.

In some embodiments, isoprene is only produced in stationary phase. In some embodiments, isoprene is produced in both the growth phase and stationary phase. In various embodiments, the amount of isoprene produced (such as the total amount of isoprene produced or the amount of isoprene produced per liter of broth per hour per OD₆₀₀) during stationary phase is greater than or about 2, 3, 4, 5, 10, 20, 30, 40, 50, or more times the amount of isoprene produced during the growth phase for the same length of time. In various embodiments, greater than or about 5, 10, 20, 30, 40, 50, 60, 70, 80, 90, 95, 99% or more of the total amount of isoprene that is produced (such as the production of isoprene during a fermentation for a certain amount of time, such as 20 hours) is produced while the cells are in stationary phase. In various embodiments, greater than or about 5, 10, 20, 30, 40, 50, 60, 70, 80, 90, 95, 99% or more of the total amount of isoprene that is produced (such as the production of isoprene during a fermentation for a certain amount of time, such as 20 hours) is produced while the cells divide slowly or not at all such that the optical density at 550 nm of the cells increases by less than or about 50% (such as by less than or about 40, 30, 20, 10, 5, or 0%). In some embodiments, isoprene is only produced in the growth phase.

In some embodiments, one or more MVA pathway, IDI, DXP, or isoprene synthase nucleic acids are placed under the control of a promoter or factor that is more active in stationary phase than in the growth phase. For example, one or more MVA pathway, IDI, DXP, or isoprene synthase nucleic acids may be placed under control of a stationary phase sigma factor, such as RpoS. In some embodiments, one or more MVA pathway, IDI, DXP, or isoprene synthase nucleic acids are placed under control of a promoter inducible in stationary phase, such as a promoter inducible by a response regulator active in stationary phase.

Production of Isoprene within Safe Operating Ranges

The production of isoprene within safe operating levels according to its flammability characteristics simplifies the design and construction of commercial facilities, vastly improves the ability to operate safely, and limits the potential for fires to occur. In particular, the optimal ranges for the production of isoprene are within the safe zone, i.e., the nonflammable range of isoprene concentrations. In one such aspect, the invention features a method for the production of isoprene within the nonflammable range of isoprene concentrations (outside the flammability envelope of isoprene).

Thus, computer modeling and experimental testing were used to determine the flammability limits of isoprene (such as isoprene in the presence of O₂, N₂, CO₂, or any combination of two or more of the foregoing gases) in order to ensure process safety. The flammability envelope is characterized by the lower flammability limit (LFL), the upper flammability limit (UFL), the limiting oxygen concentration (LOC), and the limiting temperature. For a system to be flammable, a minimum amount of fuel (such as isoprene) must be in the presence of a minimum amount of oxidant, typically oxygen. The LFL is the minimum amount of isoprene that must be present to sustain burning, while the UFL is the maximum amount of isoprene that can be present. Above this limit, the mixture is fuel rich and the fraction of oxygen is too low to have a flammable mixture. The LOC indicates the minimum fraction of oxygen that must also be present to have a flammable mixture. The limiting temperature is based on the flash point of isoprene and is that lowest temperature at which combustion of isoprene can propagate. These limits are specific to the concentration of isoprene, type and concentration of oxidant, inerts present in the system, temperature, and pressure of the system. Compositions that fall within the limits of the flammability envelope propagate combustion and require additional safety precautions in both the design and operation of process equipment.

The following conditions were tested using computer simulation and mathematical analysis and experimental testing. If desired, other conditions (such as other temperature, pressure, and permanent gas compositions) may be tested using the methods described herein to determine the LFL, UFL, and LOC concentrations.

### (1) Computer Simulation and Mathematical Analysis

### Test Suite 1:

isoprene: 0 wt %-14 wt %
O₂: 6 wt %-21 wt %
N₂: 79 wt %-94 wt %

### Test Suite 2:

isoprene: 0 wt %-14 wt %
O₂: 6 wt %-21 wt %
N₂: 79 wt %-94 wt %
   Saturated with H₂O

### Test Suite 3:

isoprene: 0 wt %-14 wt %
O₂: 6 wt %-21 wt %
N₂: 79 wt %-94 wt %
CO₂: 5 wt %-30 wt %

### (2) Experimental Testing for Final Determination of Flammability Limits

### Test Suite 1:

isoprene: 0 wt %-14 wt %
O₂: 6 wt %-21 wt %
N₂: 79 wt %-94 wt %

### Test Suite 2:

isoprene: 0 wt %-14 wt %
O₂: 6 wt %-21 wt %
N₂: 79 wt %-94 wt %
Saturated with H₂O

Simulation software was used to give an estimate of the flammability characteristics of the system for several different testing conditions. CO₂ showed no significant affect on the system's flammability limits. Test suites 1 and 2 were confirmed by experimental testing. The modeling results were in-line with the experimental test results. Only slight variations were found with the addition of water.

The LOC was determined to be 9.5 vol % for an isoprene, O₂, N₂, and CO₂ mixture at 40° C. and 1 atmosphere. The addition of up to 30% CO₂ did not significantly affect the flammability characteristics of an isoprene, O₂, and N₂ mixture. Only slight variations in flammability characteristics were shown between a dry and water saturated isoprene, O₂, and N₂ system. The limiting temperature is about −54° C. Temperatures below about −54° C. are too low to propagate combustion of isoprene.

In some embodiments, the LFL of isoprene ranges from about 1.5 vol. % to about 2.0 vol %, and the UFL of isoprene ranges from about 2.0 vol. % to about 12.0 vol. %, depending on the amount of oxygen in the system. In some embodiments, the LOC is about 9.5 vol % oxygen. In some embodiments, the LFL of isoprene is between about 1.5 vol. % to about 2.0 vol %, the UFL of isoprene is between about 2.0 vol. % to about 12.0 vol. %, and the LOC is about 9.5 vol % oxygen when the temperature is between about 25° C. to about 55° C. (such as about 40° C.) and the pressure is between about 1 atmosphere and 3 atmospheres.

In some embodiments, isoprene is produced in the presence of less than about 9.5 vol % oxygen (that is, below the LOC required to have a flammable mixture of isoprene). In some embodiments in which isoprene is produced in the presence of greater than or about 9.5 vol % oxygen, the isoprene concentration is below the LFL (such as below about 1.5 vol. %). For example, the amount of isoprene can be kept below the LFL by diluting the isoprene composition with an inert gas (e.g., by continuously or periodically adding an inert gas such as nitrogen to keep the isoprene composition below the LFL). In some embodiments in which isoprene is produced in the presence of greater than or about 9.5 vol % oxygen, the isoprene concentration is above the UFL (such as above about 12 vol. %). For example, the amount of isoprene can be kept above the UFL by using a system (such as any of the cell culture systems described herein) that produces isoprene at a concentration above the UFL. If desired, a relatively low level of oxygen can be used so that the UFL is also relatively low. In this case, a lower isoprene concentration is needed to remain above the UFL.

In some embodiments in which isoprene is produced in the presence of greater than or about 9.5 vol % oxygen, the isoprene concentration is within the flammability envelope (such as between the LFL and the UFL). In some embodiments when the isoprene concentration may fall within the flammability envelope, one or more steps are performed to reduce the probability of a fire or explosion. For example, one or more sources of ignition (such as any materials that may generate a spark) can be avoided. In some embodiments, one or more steps are performed to reduce the amount of time that the concentration of isoprene remains within the flammability envelope. In some embodiments, a sensor is used to detect when the concentration of isoprene is close to or within the flammability envelope. If desired, the concentration of isoprene can be measured at one or more time points during the culturing of cells, and the cell culture conditions and/or the amount of inert gas can be adjusted using standard methods if the concentration of isoprene is close to or within the flammability envelope. In particular embodiments, the cell culture conditions (such as fermentation conditions) are adjusted to either decrease the concentration of isoprene below the LFL or increase the concentration of isoprene above the UFL. In some embodiments, the amount of isoprene is kept below the LFL by diluting the isoprene composition with an inert gas (such as by continuously or periodically adding an inert gas to keep the isoprene composition below the LFL).

In some embodiments, the amount of flammable volatiles other than isoprene (such as one or more sugars) is at least about 2, 5, 10, 50, 75, or 100-fold less than the amount of isoprene produced. In some embodiments, the portion of the gas phase other than isoprene gas comprises between about 0% to about 100% (volume) oxygen, such as between about 0% to about 10%, about 10% to about 20%, about 20% to about 30%, about 30% to about 40%, about 40% to about 50%, about 50% to about 60%, about 60% to about 70%, about 70% to about 80%, about 90% to about 90%, or about 90% to about 100% (volume) oxygen. In some embodiments, the portion of the gas phase other than isoprene gas comprises between about 0% to about 99% (volume) nitrogen, such as between about 0% to about 10%, about 10% to about 20%, about 20% to about 30%, about 30% to about 40%, about 40% to about 50%, about 50% to about 60%, about 60% to about 70%, about 70% to about 80%, about 90% to about 90%, or about 90% to about 99% (volume) nitrogen.

In some embodiments, the portion of the gas phase other than isoprene gas comprises between about 1% to about 50% (volume) CO₂, such as between about 1% to about 10%, about 10% to about 20%, about 20% to about 30%, about 30% to about 40%, or about 40% to about 50% (volume) CO₂.

In some embodiments, an isoprene composition also contains ethanol. For example, ethanol may be used for extractive distillation of isoprene, resulting in compositions (such as intermediate product streams) that include both ethanol and isoprene. Desirably, the amount of ethanol is outside the flammability envelope for ethanol. The LOC of ethanol is about 8.7 vol %, and the LFL for ethanol is about 3.3 vol % at standard conditions, such as about 1 atmosphere and about 60° F. (NFPA 69 Standard on Explosion Prevention Systems, 2008 edition, which is hereby incorporated by reference in its entirety, particularly with respect to LOC, LFL, and UFL values). In some embodiments, compositions that include isoprene and ethanol are produced in the presence of less than the LOC required to have a flammable mixture of ethanol (such as less than about 8.7% vol %). In some embodiments in which compositions that include isoprene and ethanol are produced in the presence of greater than or about the LOC required to have a flammable mixture of ethanol, the ethanol concentration is below the LFL (such as less than about 3.3 vol. %).

In various embodiments, the amount of oxidant (such as oxygen) is below the LOC of any fuel in the system (such as isoprene or ethanol). In various embodiments, the amount of oxidant (such as oxygen) is less than about 60, 40, 30, 20, 10, or 5% of the LOC of isoprene or ethanol. In various embodiments, the amount of oxidant (such as oxygen) is less than the LOC of isoprene or ethanol by at least 2, 4, 5, or more absolute percentage points (vol %). In particular embodiments, the amount of oxygen is at least 2 absolute percentage points (vol %) less than the LOC of isoprene or ethanol (such as an oxygen concentration of less than 7.5 vol % when the LOC of isoprene is 9.5 vol %). In various embodiments, the amount of fuel (such as isoprene or ethanol) is less than or about 25, 20, 15, 10, or 5% of the LFL for that fuel.

### Exemplary Production of Isoprene

In some embodiments, the cells are cultured in a culture medium under conditions permitting the production of isoprene by the cells. By “peak absolute productivity” is meant the maximum absolute amount of isoprene in the off-gas during the culturing of cells for a particular period of time (e.g., the culturing of cells during a particular fermentation run). By “peak absolute productivity time point” is meant the time point during a fermentation run when the absolute amount of isoprene in the off-gas is at a maximum during the culturing of cells for a particular period of time (e.g., the culturing of cells during a particular fermentation run). In some embodiments, the isoprene amount is measured at the peak absolute productivity time point. In some embodiments, the peak absolute productivity for the cells is about any of the isoprene amounts disclosed herein.

By “peak specific productivity” is meant the maximum amount of isoprene produced per cell during the culturing of cells for a particular period of time (e.g., the culturing of cells during a particular fermentation run). By “peak specific productivity time point” is meant the time point during the culturing of cells for a particular period of time (e.g., the culturing of cells during a particular fermentation run) when the amount of isoprene produced per cell is at a maximum. The specific productivity is determined by dividing the total productivity by the amount of cells, as determined by optical density at 600 nm (OD600). In some embodiments, the isoprene amount is measured at the peak specific productivity time point. In some embodiments, the peak specific productivity for the cells is about any of the isoprene amounts per cell disclosed herein.

By “cumulative total productivity” is meant the cumulative, total amount of isoprene produced during the culturing of cells for a particular period of time (e.g., the culturing of cells during a particular fermentation run). In some embodiments, the cumulative, total amount of isoprene is measured. In some embodiments, the cumulative total productivity for the cells is about any of the isoprene amounts disclosed herein.

By “relative detector response” refers to the ratio between the detector response (such as the GC/MS area) for one compound (such as isoprene) to the detector response (such as the GC/MS area) of one or more compounds (such as all C5 hydrocarbons). The detector response may be measured as described herein, such as the GC/MS analysis performed with an Agilent 6890 GC/MS system fitted with an Agilent HP-5MS GC/MS column (30 m×250 μm; 0.25 μm film thickness). If desired, the relative detector response can be converted to a weight percentage using the response factors for each of the compounds. This response factor is a measure of how much signal is generated for a given amount of a particular compound (that is, how sensitive the detector is to a particular compound). This response factor can be used as a correction factor to convert the relative detector response to a weight percentage when the detector has different sensitivities to the compounds being compared. Alternatively, the weight percentage can be approximated by assuming that the response factors are the same for the compounds being compared. Thus, the weight percentage can be assumed to be approximately the same as the relative detector response.

In some embodiments, the cells in culture produce isoprene at greater than or about 1, 10, 25, 50, 100, 150, 200, 250, 300, 400, 500, 600, 700, 800, 900, 1,000, 1,250, 1,500, 1,750, 2,000, 2,500, 3,000, 4,000, 5,000, or more nmole of isoprene/gram of cells for the wet weight of the cells/hour (nmole/g_{wcm}/hr). In some embodiments, the amount of isoprene is between about 2 to about 5,000 nmole/g_{wcm}/hr, such as between about 2 to about 100 nmole/g_{wcm}/hr, about 100 to about 500 nmole/g_{wcm}/hr, about 150 to about 500 nmole/g_{wcm}/hr, about 500 to about 1,000 nmole/g_{wcm}/hr, about 1,000 to about 2,000 nmole/g_{wcm}/hr, or about 2,000 to about 5,000 nmole/g_{wcm}/hr. In some embodiments, the amount of isoprene is between about 20 to about 5,000 nmole/g_{wcm}/hr, about 100 to about 5,000 nmole/g_{wcm}/hr, about 200 to about 2,000 nmole/g_{wcm}/hr, about 200 to about 1,000 nmole/g_{wcm}/hr, about 300 to about 1,000 nmole/g_{wcm}/hr, or about 400 to about 1,000 nmole/g_{wcm}/hr.

The amount of isoprene in units of nmole/g_{wcm}/hr can be measured as disclosed in U.S. Pat. No. 5,849,970, which is hereby incorporated by reference in its entirety, particularly with respect to the measurement of isoprene production. For example, two mL of headspace (e.g., headspace from a culture such as 2 mL of culture cultured in sealed vials at 32° C. with shaking at 200 rpm for approximately 3 hours) are analyzed for isoprene using a standard gas chromatography system, such as a system operated isothermally (85° C.) with an n-octane/porasil C column (Alltech Associates, Inc., Deerfield, Ill.) and coupled to a RGD2 mercuric oxide reduction gas detector (Trace Analytical, Menlo Park, Calif.) (see, for example, Greenberg et al, Atmos. Environ. 27A: 2689-2692, 1993; Silver et al., Plant Physiol. 97:1588-1591, 1991, which are each hereby incorporated by reference in their entireties, particularly with respect to the measurement of isoprene production). The gas chromatography area units are converted to nmol isoprene via a standard isoprene concentration calibration curve. In some embodiments, the value for the grams of cells for the wet weight of the cells is calculated by obtaining the A₆₀₀ value for a sample of the cell culture, and then converting the A₆₀₀ value to grams of cells based on a calibration curve of wet weights for cell cultures with a known A₆₀₀ value. In some embodiments, the grams of the cells is estimated by assuming that one liter of broth (including cell medium and cells) with an A₆₀₀ value of 1 has a wet cell weight of 1 gram. The value is also divided by the number of hours the culture has been incubating for, such as three hours.

In some embodiments, the cells in culture produce isoprene at greater than or about 1, 10, 25, 50, 100, 150, 200, 250, 300, 400, 500, 600, 700, 800, 900, 1,000, 1,250, 1,500, 1,750, 2,000, 2,500, 3,000, 4,000, 5,000, 10,000, 100,000, or more ng of isoprene/gram of cells for the wet weight of the cells/hr (ng/g_{wcm}/h). In some embodiments, the amount of isoprene is between about 2 to about 5,000 ng/g_{wcm}/h, such as between about 2 to about 100 ng/g_{wcm}/h, about 100 to about 500 ng/g_{wcm}/h, about 500 to about 1,000 ng/g_{wcm}/h, about 1,000 to about 2,000 ng/g_{wcm}/h, or about 2,000 to about 5,000 ng/g_{wcm}/h. In some embodiments, the amount of isoprene is between about 20 to about 5,000 ng/g_{wcm}/h, about 100 to about 5,000 ng/g_{wcm}/h, about 200 to about 2,000 ng/g_{wcm}/h, about 200 to about 1,000 ng/g_{wcm}/h, about 300 to about 1,000 ng/g_{wcm}/h, or about 400 to about 1,000 ng/g_{wcm}/h. The amount of isoprene in ng/g_{wcm}/h can be calculated by multiplying the value for isoprene production in the units of nmole/g_{wcm}/hr discussed above by 68.1 (as described in Equation 5 below).

In some embodiments, the cells in culture produce a cumulative titer (total amount) of isoprene at greater than or about 1, 10, 25, 50, 100, 150, 200, 250, 300, 400, 500, 600, 700, 800, 900, 1,000, 1,250, 1,500, 1,750, 2,000, 2,500, 3,000, 4,000, 5,000, 10,000, 50,000, 100,000, or more mg of isoprene/L of broth (mg/L_{broth}, wherein the volume of broth includes the volume of the cells and the cell medium). In some embodiments, the amount of isoprene is between about 2 to about 5,000 mg/L_{broth}, such as between about 2 to about 100 mg/L_{broth}, about 100 to about 500 mg/L_{broth}, about 500 to about 1,000 mg/L_{broth}, about 1,000 to about 2,000 mg/L_{broth}, or about 2,000 to about 5,000 mg/L_{broth}. In some embodiments, the amount of isoprene is between about 20 to about 5,000 mg/L_{broth}, about 100 to about 5,000 mg/L_{broth}, about 200 to about 2,000 mg/L_{broth}, about 200 to about 1,000 mg/L_{broth}, about 300 to about 1,000 mg/L_{broth}, or about 400 to about 1,000 mg/L_{broth}.

The specific productivity of isoprene in mg of isoprene/L of headspace from shake flask or similar cultures can be measured by taking a 1 ml sample from the cell culture at an 0D₆₀₀ value of approximately 1.0, putting it in a 20 mL vial, incubating for 30 minutes, and then measuring the amount of isoprene in the headspace (as described, for example, in Example 13, part II). If the OD₆₀₀ value is not 1.0, then the measurement can be normalized to an OD₆₀₀ value of 1.0 by dividing by the OD₆₀₀ value. The value of mg isoprene/L headspace can be converted to mg/L_{broth}/hr/OD₆₀₀ of culture broth by multiplying by a factor of 38. The value in units of mg/L_{broth}/hr/OD₆₀₀ can be multiplied by the number of hours and the OD₆₀₀ value to obtain the cumulative titer in units of mg of isoprene/L of broth.

The instantaneous isoprene production rate in mg/L_{broth}/hr in a fermentor can be measured by taking a sample of the fermentor off-gas, analyzing it for the amount of isoprene (in units such as mg of isoprene per L_{gas}) as described, for example, in Example 13, part II and multiplying this value by the rate at which off-gas is passed though each liter of broth (e.g., at 1 vvm (volume of air/volume of broth/minute) this is 60 L_{gas} per hour). Thus, an off-gas level of 1 mg/L_{gas} corresponds to an instantaneous production rate of 60 mg/L_{broth}/hr at air flow of 1 vvm. If desired, the value in the units mg/L_{broth}/hr can be divided by the OD₆₀₀ value to obtain the specific rate in units of mg/L_{broth}/hr/OD. The average value of mg isoprene/L_{gas} can be converted to the total product productivity (grams of isoprene per liter of fermentation broth, mg/L_{broth}) by multiplying this average off-gas isoprene concentration by the total amount of off-gas sparged per liter of fermentation broth during the fermentation. Thus, an average off-gas isoprene concentration of 0.5 mg/L_{broth}/hr over 10 hours at 1 vvm corresponds to a total product concentration of 300 mg isoprene/L_{broth}.

In some embodiments, the cells in culture convert greater than or about 0.0015, 0.002, 0.005, 0.01, 0.02, 0.05, 0.1, 0.12, 0.14, 0.16, 0.2, 0.3, 0.4, 0.5, 0.6, 0.7, 0.8, 0.9, 1.0, 1.2, 1.4, 1.6, 1.8, 2.0, 2.5, 3.0, 3.5, 4.0, 5.0, 6.0, 7.0, or 8.0% of the carbon in the cell culture medium into isoprene. In some embodiments, the percent conversion of carbon into isoprene is between such as about 0.002 to about 4.0%, about 0.002 to about 3.0%, about 0.002 to about 2.0%, about 0.002 to about 1.6%, about 0.002 to about 0.005%, about 0.005 to about 0.01%, about 0.01 to about 0.05%, about 0.05 to about 0.15%, 0.15 to about 0.2%, about 0.2 to about 0.3%, about 0.3 to about 0.5%, about 0.5 to about 0.8%, about 0.8 to about 1.0%, or about 1.0 to about 1.6%. In some embodiments, the percent conversion of carbon into isoprene is between about 0.002 to about 0.4%, 0.002 to about 0.16%, 0.04 to about 0.16%, about 0.005 to about 0.3%, about 0.01 to about 0.3%, or about 0.05 to about 0.3%.

The percent conversion of carbon into isoprene (also referred to as “% carbon yield”) can be measured by dividing the moles carbon in the isoprene produced by the moles carbon in the carbon source (such as the moles of carbon in batched and fed glucose and yeast extract). This number is multiplied by 100% to give a percentage value (as indicated in Equation 1).

Carbon Yield=(moles carbon in isoprene produced)/(moles carbon in carbon source)*100 Equation 1%

For this calculation, yeast extract can be assumed to contain 50% w/w carbon. As an example, for the 500 liter described in Example 19, part VIII, the percent conversion of carbon into isoprene can be calculated as shown in Equation 2.

Carbon Yield=(39.1 g isoprene*1/68.1mol/g*5 C/mol)/[(181221 g glucose*1/180 mol/g*6 C/mol)+(17780 g yeast extract*0.5*1/12 mol/g)]*100=0.042% Equation 2%

For the two 500 liter fermentations described herein (Example 19, parts VII and VIII), the percent conversion of carbon into isoprene was between 0.04-0.06%. A 0.11-0.16% carbon yield has been achieved using 14 liter systems as described herein. Example 22, part V describes the 1.53% conversion of carbon to isoprene using the methods described herein.

One skilled in the art can readily convert the rates of isoprene production or amount of isoprene produced into any other units. Exemplary equations are listed below for interconverting between units.

Units for Rate of Isoprene production (total and specific)

1 g isoprene/L_{broth}/hr=14.7 mmol isoprene/L_{broth}/hr (total volumetric rate) Equation 3%

1 nmol isoprene/g_{wcm}/hr=1 nmol isoprene/L_{broth}/hr/OD₆₀₀ (This conversion assumes that one liter of broth with an OD₆₀₀ value of 1 has a wet cell weight of 1 gram.) Equation 4%

1 nmol isoprene/g_{wcm}/hr=68.1 ng isoprene/g_{wcm}/hr (given the molecular weight of isoprene) Equation 5%

1 nmol isoprene/L_{gas} O₂/hr=90 nmol isoprene/L_{broth}/hr (at an O₂ flow rate of 90 L/hr per L of culture broth) Equation 6%

1 ug isoprene/L_{gas} isoprene in off-gas=60 ug isoprene/L_{broth}/hr at a flow rate of 60 L_{gas} per L_{broth} (1 vvm) Equation 7%

Units for Titer (total and specific)

1 nmol isoprene/mg cell protein=150 nmol isoprene/L_{broth}/OD₆₀₀ (This conversion assumes that one liter of broth with an OD₆₀₀ value of 1 has a total cell protein of approximately 150 mg) (specific productivity) Equation 8%

1 g isoprene/L_{broth}=14.7 mmol isoprene/L_{broth} (total titer) Equation 9%

If desired, Equation 10 can be used to convert any of the units that include the wet weight of the cells into the corresponding units that include the dry weight of the cells.

Dry weight of cells=(wet weight of cells)/3.3 Equation 10%

If desired, Equation 11 can be used to convert between units of ppm and ug/L. In particular, “ppm” means parts per million defined in terms of ug/g (w/w). Concentrations of gases can also be expressed on a volumetric basis using “ppmv” (parts per million by volume), defined in terms of uL/L (vol/vol). Conversion of ug/L to ppm (e.g., ug of analyte per g of gas) can be performed by determining the mass per L of off-gas (i.e., the density of the gas). For example, a liter of air at standard temperature and pressure (STP; 101.3 kPa (1 bar) and 273.15K) has a density of approximately 1.29 g/L. Thus, a concentration of 1 ppm (ug/g) equals 1.29 ug/L at STP (equation 11). The conversion of ppm (ug/g) to ug/L is a function of both pressure, temperature, and overall composition of the off-gas.

1 ppm (ug/g) equals 1.29 ug/L at standard temperature and pressure (STP; 101.3 kPa (1 bar) and 273.15K). Equation 11

Conversion of ug/L to ppmv (e.g., uL of analyte per L of gas) can be performed using the Universal Gas Law (equation 12). For example, an off-gas concentration of 1000 ug/L_{gas} corresponds to 14.7 umol/L_{gas}. The universal gas constant is 0.082057 L.atm K⁻¹ mol⁻¹, so using equation 12, the volume occupied by 14.7 umol of HG at STP is equal to 0.329 mL. Therefore, the concentration of 1000 ug/L HG is equal to 329 ppmv or 0.0329% (v/v) at STP.

PV=nRT, where “P” is pressure, “V” is volume, “n” is moles of gas, “R” is the Universal gas constant, and “T” is temperature in Kelvin. Equation 12

The amount of impurities in isoprene compositions are typically measured herein on a weight per volume (w/v) basis in units such as ug/L. If desired, measurements in units of ug/L can be converted to units of mg/m³ using equation 13.

1 ug/L=1 mg/m³ Equation 13

In some embodiments encompassed by the invention, a cell comprising a heterologous nucleic acid encoding an isoprene synthase polypeptide produces an amount of isoprene that is at least or about 2-fold, 3-fold, 5-fold, 10-fold, 25-fold, 50-fold, 100-fold, 150-fold, 200-fold, 400-fold, or greater than the amount of isoprene produced from a corresponding cell grown under essentially the same conditions without the heterologous nucleic acid encoding the isoprene synthase polypeptide.

In some embodiments encompassed by the invention, a cell comprising a heterologous nucleic acid encoding an isoprene synthase polypeptide and one or more heterologous nucleic acids encoding a DXS, IDI, and/or MVA pathway polypeptide produces an amount of isoprene that is at least or about 2-fold, 3-fold, 5-fold, 10-fold, 25-fold, 50-fold, 100-fold, 150-fold, 200-fold, 400-fold, or greater than the amount of isoprene produced from a corresponding cell grown under essentially the same conditions without the heterologous nucleic acids.

In some embodiments, the isoprene composition comprises greater than or about 99.90, 99.92, 99.94, 99.96, 99.98, or 100% isoprene by weight compared to the total weight of all C5 hydrocarbons in the composition. In some embodiments, the composition has a relative detector response of greater than or about 99.90, 99.91, 99.92, 99.93, 99.94, 99.95, 99.96, 99.97, 99.98, 99.99, or 100% for isoprene compared to the detector response for all C5 hydrocarbons in the composition. In some embodiments, the isoprene composition comprises between about 99.90 to about 99.92, about 99.92 to about 99.94, about 99.94 to about 99.96, about 99.96 to about 99.98, about 99.98 to 100% isoprene by weight compared to the total weight of all C5 hydrocarbons in the composition.

In some embodiments, the isoprene composition comprises less than or about 0.12, 0.10, 0.08, 0.06, 0.04, 0.02, 0.01, 0.005, 0.001, 0.0005, 0.0001, 0.00005, or 0.00001% C5 hydrocarbons other than isoprene (such 1,3-cyclopentadiene, trans-1,3-pentadiene, cis-1,3-pentadiene, 1,4-pentadiene, 1-pentyne, 2-pentyne, 3-methyl-1-butyne, pent-4-ene-1-yne, trans-pent-3-ene-1-yne, cis-pent-3-ene-1-yne, 3-hexen-1-ol, 3-hexen-1-y1 acetate, limonene, geraniol (trans-3,7-dimethyl-2,6-octadien-1-ol) and citronellol (3,7-dimethyl-6-octen-1-ol)) by weight compared to the total weight of all C5 hydrocarbons in the composition. In some embodiments, the composition has a relative detector response of less than or about 0.12, 0.10, 0.08, 0.06, 0.04, 0.02, 0.01, 0.005, 0.001, 0.0005, 0.0001, 0.00005, or 0.00001% for C5 hydrocarbons other than isoprene compared to the detector response for all C5 hydrocarbons in the composition. In some embodiments, the composition has a relative detector response of less than or about 0.12, 0.10, 0.08, 0.06, 0.04, 0.02, 0.01, 0.005, 0.001, 0.0005, 0.0001, 0.00005, or 0.00001% for 1,3-cyclopentadiene, 1,3-cyclopentadiene, trans-1,3-pentadiene, cis-1,3-pentadiene, 1,4-pentadiene, 1-pentyne, 2-pentyne, 3-methyl-1-butyne, pent-4-ene-1-yne, trans-pent-3-ene-1-yne, cis-pent-3-ene-1-yne, 3-hexen-1-ol, 3-hexen-1-yl acetate, limonene, geraniol (trans-3,7-dimethyl-2,6-octadien-1-ol) and citronellol (3,7-dimethyl-6-octen-1-ol) compared to the detector response for all C5 hydrocarbons in the composition. In some embodiments, the isoprene composition comprises between about 0.02 to about 0.04%, about 0.04 to about 0.06%, about 0.06 to 0.08%, about 0.08 to 0.10%, or about 0.10 to about 0.12% C5 hydrocarbons other than isoprene (such as 1,3-cyclopentadiene, trans-1,3-pentadiene, cis-1,3-pentadiene, 1,4-pentadiene, 1-pentyne, 2-pentyne, 3-methyl-1-butyne, pent-4-ene-1-yne, trans-pent-3-ene-1-yne, cis-pent-3-ene-1-yne, 3-hexen-1-ol, 3-hexen-1-yl acetate, limonene, geraniol (trans-3,7-dimethyl-2,6-octadien-1-ol) and citronellol (3,7-dimethyl-6-octen-1-ol)) by weight compared to the total weight of all C5 hydrocarbons in the composition.

In some embodiments, the isoprene composition comprises less than or about 50, 40, 30, 20, 10, 5, 1, 0.5, 0.1, 0.05, 0.01, or 0.005 ug/L of a compound that inhibits the polymerization of isoprene for any compound in the composition that inhibits the polymerization of isoprene. In some embodiments, the isoprene composition comprises between about 0.005 to about 50, such as about 0.01 to about 10, about 0.01 to about 5, about 0.01 to about 1, about 0.01 to about 0.5, or about 0.01 to about 0.005 ug/L of a compound that inhibits the polymerization of isoprene for any compound in the composition that inhibits the polymerization of isoprene. In some embodiments, the isoprene composition comprises less than or about 50, 40, 30, 20, 10, 5, 1, 0.5, 0.1, 0.05, 0.01, or 0.005 ug/L of a hydrocarbon other than isoprene (such as 1,3-cyclopentadiene, trans-1,3-pentadiene, cis-1,3-pentadiene, 1,4-pentadiene, 1-pentyne, 2-pentyne, 3-methyl-1-butyne, pent-4-ene-1-yne, trans-pent-3-ene-1-yne, cis-pent-3-ene-1-yne, 3-hexen-1-ol, 3-hexen-1-yl acetate, limonene, geraniol (trans-3,7-dimethyl-2,6-octadien-1-ol) and citronellol (3,7-dimethyl-6-octen-1-ol)). In some embodiments, the isoprene composition comprises between about 0.005 to about 50, such as about 0.01 to about 10, about 0.01 to about 5, about 0.01 to about 1, about 0.01 to about 0.5, or about 0.01 to about 0.005 ug/L of a hydrocarbon other than isoprene. In some embodiments, the isoprene composition comprises less than or about 50, 40, 30, 20, 10, 5, 1, 0.5, 0.1, 0.05, 0.01, or 0.005 ug/L of a protein or fatty acid (such as a protein or fatty acid that is naturally associated with natural rubber).

In some embodiments, the isoprene composition comprises less than or about 10, 5, 1, 0.8, 0.5, 0.1, 0.05, 0.01, or 0.005 ppm of alpha acetylenes, piperylenes, acetonitrile, or 1,3-cyclopentadiene. In some embodiments, the isoprene composition comprises less than or about 5, 1, 0.5, 0.1, 0.05, 0.01, or 0.005 ppm of sulfur or allenes. In some embodiments, the isoprene composition comprises less than or about 30, 20, 15, 10, 5, 1, 0.5, 0.1, 0.05, 0.01, or 0.005 ppm of all acetylenes (such as pentyne-1, butyne-2, 2MB1-3yne, and 1-pentyne-4yne). In some embodiments, the isoprene composition comprises less than or about 2000, 1000, 500, 200, 100, 50, 40, 30, 20, 10, 5, 1, 0.5, 0.1, 0.05, 0.01, or 0.005 ppm of isoprene dimers, such as cyclic isoprene dimmers (e.g., cyclic C10 compounds derived from the dimerization of two isoprene units).

In some embodiments, the composition comprises greater than about 2 mg of isoprene, such as greater than or about 5, 10, 20, 30, 40, 50, 60, 70, 80, 90, 100, 200, 300, 400, 500, 600, 700, 800, 900, or 1000 mg of isoprene. In some embodiments, the composition comprises greater than or about 2, 5, 10, 20, 30, 40, 50, 60, 70, 80, 90, 100 g of isoprene. In some embodiments, the amount of isoprene in the composition is between about 2 to about 5,000 mg, such as between about 2 to about 100 mg, about 100 to about 500 mg, about 500 to about 1,000 mg, about 1,000 to about 2,000 mg, or about 2,000 to about 5,000 mg. In some embodiments, the amount of isoprene in the composition is between about 20 to about 5,000 mg, about 100 to about 5,000 mg, about 200 to about 2,000 mg, about 200 to about 1,000 mg, about 300 to about 1,000 mg, or about 400 to about 1,000 mg. In some embodiments, greater than or about 20, 25, 30, 40, 50, 60, 70, 80, 90, or 95% by weight of the volatile organic fraction of the composition is isoprene.

In some embodiments, the composition includes ethanol. In some embodiments, the composition includes between about 75 to about 90% by weight of ethanol, such as between about 75 to about 80%, about 80 to about 85%, or about 85 to about 90% by weight of ethanol. In some embodiments in which the composition includes ethanol, the composition also includes between about 4 to about 15% by weight of isoprene, such as between about 4 to about 8%, about 8 to about 12%, or about 12 to about 15% by weight of isoprene.

### Exemplary Isoprene Purification Methods

In some embodiments, any of the methods described herein further include recovering the isoprene. For example, the isoprene produced using the compositions and methods of the invention can be recovered using standard techniques such as gas stripping, membrane enhanced separation, fractionation, adsorption/desorption, pervaporation, thermal or vacuum desorption of isoprene from a solid phase, or extraction of isoprene immobilized or absorbed to a solid phase with a solvent (see, for example, U.S. Pat. Nos. 4,703,007 and 4,570,029, which are each hereby incorporated by reference in their entireties, particularly with respect to isoprene recovery and purification methods). In particular, embodiments, extractive distillation with an alcohol (such as ethanol, methanol, propanol, or a combination thereof) is used to recover the isoprene. In some embodiments, the recovery of isoprene involves the isolation of isoprene in a liquid form (such as a neat solution of isoprene or a solution of isoprene in a solvent). Gas stripping involves the removal of isoprene vapor from the fermentation off-gas stream in a continuous manner. Such removal can be achieved in several different ways including, but not limited to, adsorption to a solid phase, partition into a liquid phase, or direct condensation (such as condensation due to exposure to a condensation coil or do to an increase in pressure). In some embodiments, membrane enrichment of a dilute isoprene vapor stream above the dew point of the vapor resulting in the condensation of liquid isoprene. In some embodiments, the isoprene is compressed and condensed.

The recovery of isoprene may involve one step or multiple steps. In some embodiments, the removal of isoprene vapor from the fermentation off-gas and the conversion of isoprene to a liquid phase are performed simultaneously. For example, isoprene can be directly condensed from the off-gas stream to form a liquid. In some embodiments, the removal of isoprene vapor from the fermentation off-gas and the conversion of isoprene to a liquid phase are performed sequentially. For example, isoprene may be adsorbed to a solid phase and then extracted from the solid phase with a solvent.

In some embodiments, any of the methods described herein further include purifying the isoprene. For example, the isoprene produced using the compositions and methods of the invention can be purified using standard techniques. Purification refers to a process through which isoprene is separated from one or more components that are present when the isoprene is produced. In some embodiments, the isoprene is obtained as a substantially pure liquid. Examples of purification methods include (i) distillation from a solution in a liquid extractant and (ii) chromatography. As used herein, “purified isoprene” means isoprene that has been separated from one or more components that are present when the isoprene is produced. In some embodiments, the isoprene is at least about 20%, by weight, free from other components that are present when the isoprene is produced. In various embodiments, the isoprene is at least or about 25%, 30%, 40%, 50%, 60%, 70%, 75%, 80%, 90%, 95%, or 99%, by weight, pure. Purity can be assayed by any appropriate method, e.g., by column chromatography, HPLC analysis, or GC-MS analysis.

In some embodiments, at least a portion of the gas phase remaining after one or more recovery steps for the removal of isoprene is recycled by introducing the gas phase into a cell culture system (such as a fermentor) for the production of isoprene.

In some embodiments, any of the methods described herein further include polymerizing the isoprene. For example, standard methods can be used to polymerize the purified isoprene to form cis-polyisoprene or other down stream products using standard methods. Accordingly, the invention also features a tire comprising polyisoprene, such as cis-1,4-polyisoprene and/or trans-1,4-polyisoprene made from any of the isoprene compositions disclosed herein.

The following Examples are provided to illustrate but not limit the invention.

### Examples

The examples, which are intended to be purely exemplary of the invention and should therefore not be considered to limit the invention in any way, also describe and detail aspects and embodiments of the invention discussed above. Unless indicated otherwise, temperature is in degrees Centigrade and pressure is at or near atmospheric. The foregoing examples and detailed description are offered by way of illustration and not by way of limitation. All publications, patent applications, and patents cited in this specification are herein incorporated by reference as if each individual publication, patent application, or patent were specifically and individually indicated to be incorporated by reference. In particular, all publications cited herein are expressly incorporated herein by reference for the purpose of describing and disclosing compositions and methodologies which might be used in connection with the invention. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

### Example 1

### Expression Constructs and Strains

### I. Construction of Plasmids Encoding Mevalonate Kinase.

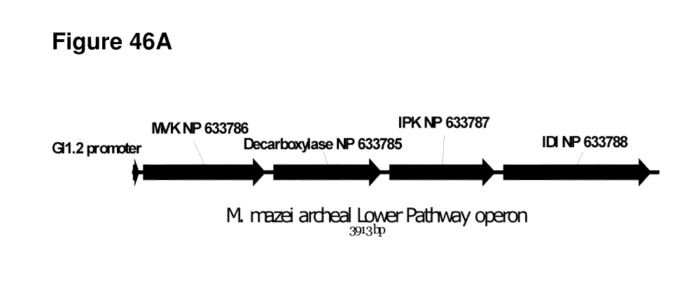

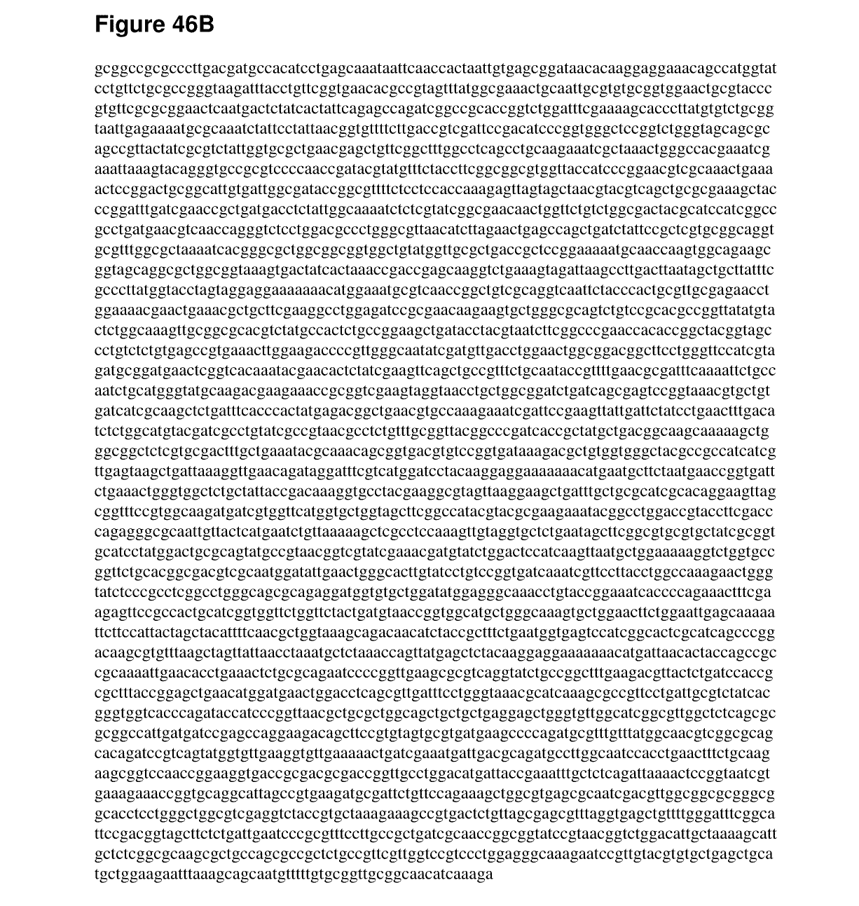

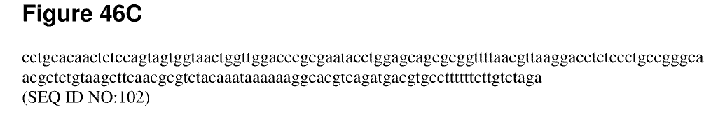

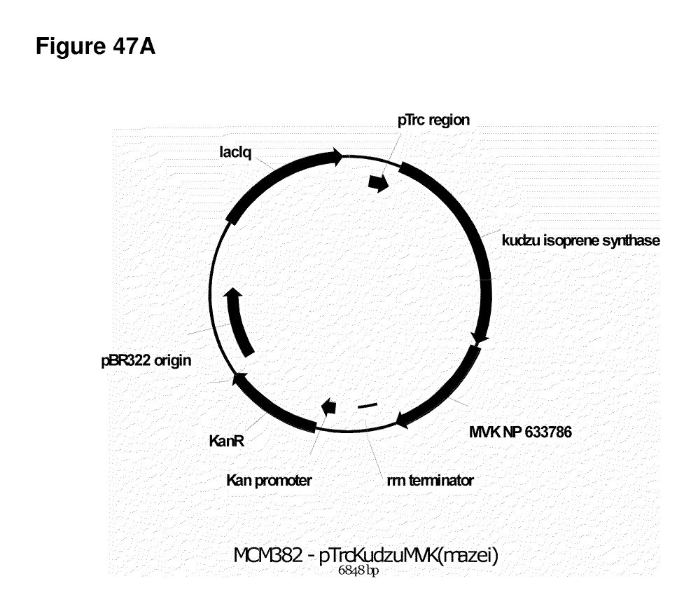

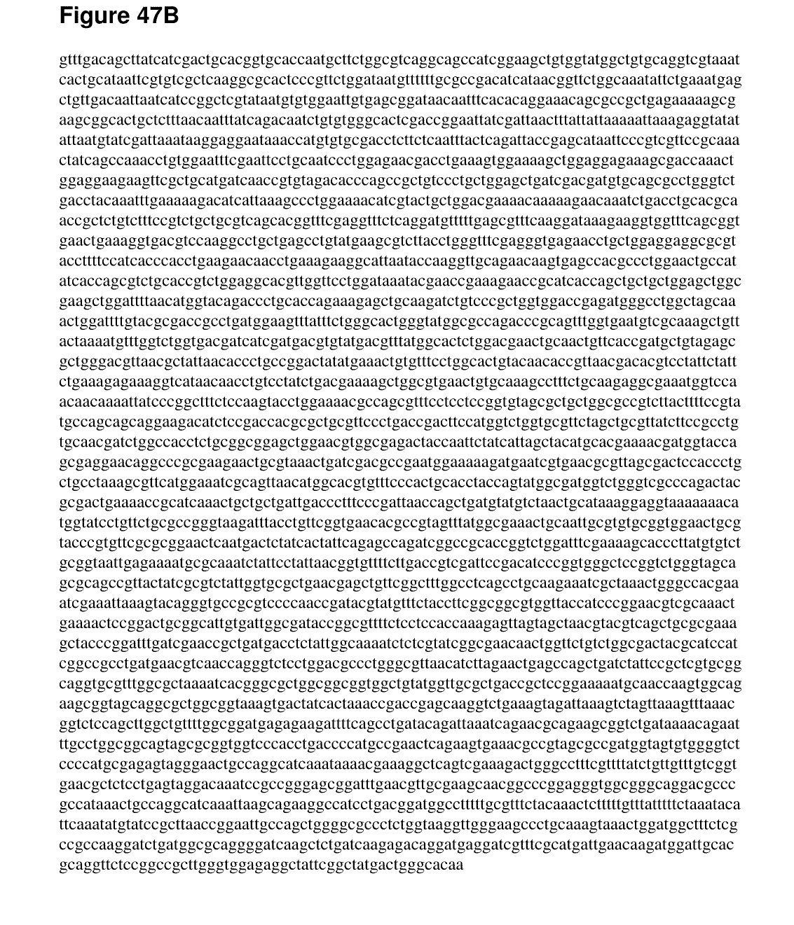

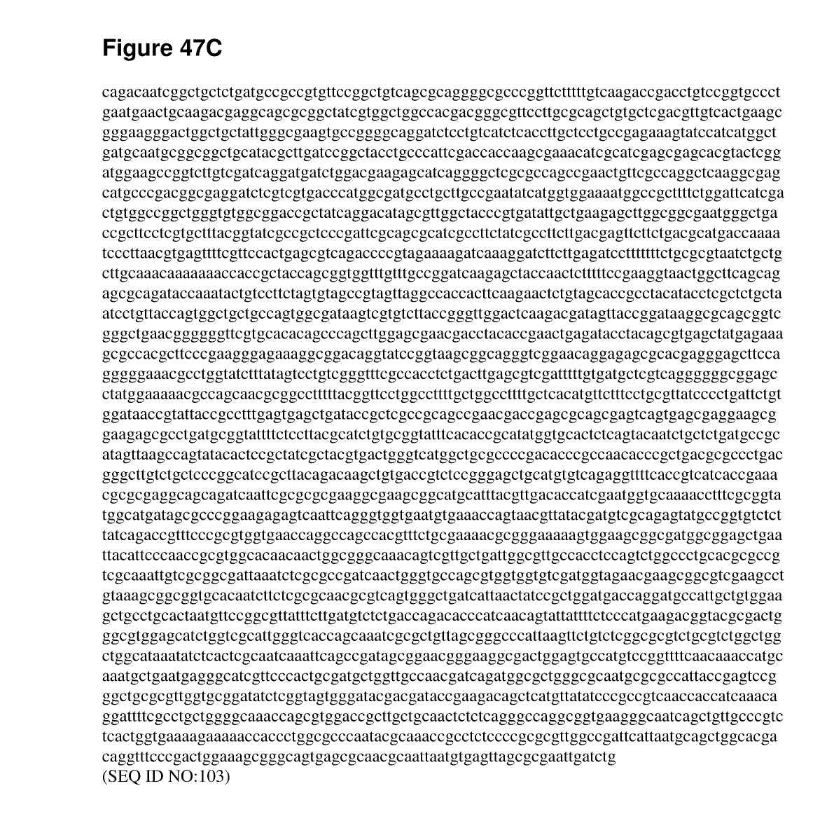

A construct encoding the Methanosarcina mazei lower MVA pathway (Accession numbers NC_{—}003901.1, NC_{—}003901.1, NC_{—}003901.1, and NC_{—}003901.1, which are each hereby incorporated by reference in their entireties) was synthesized with codon optimization for expression in E. coli. This construct is named M. mazei archaeal Lower Pathway operon (FIGS. 46A-46C) and encodes M. mazei MVK, a putative decarboxylase, IPK, and IDI enzymes. The gene encoding MVK (Accession number NC_{—}003901.1) was PCR amplified using primers MCM165 and MCM177 (Table 4) using the Strategene Herculase II Fusion kit according to the manufacturer's protocol using 30 cycles with an annealing temperature of 55° C. and extension time of 60 seconds. This amplicon was purified using a Qiagen PCR column and then digested at 37° C. in a 10 uL reaction with PmeI (in the presence of NEB buffer 4 and BSA). After one hour, NsiI and Roche buffer H were added for an additional hour at 37° C. The digested DNA was purified over a Qiagen PCR column and ligated to a similarly digested and purified plasmid MCM29 in an 11 uL reaction 5 uL Roche Quick Ligase buffer 1, 1 uL buffer 2, 1 uL plasmid, 3 uL amplicon, and 1 uL ligase (1 hour at room temperature). MCM 29 is pTrcKudzuKan. The ligation reaction was introduced into Invitrogen TOP10 cells and transformants selected on LA/kan50 plates incubated at 37° C. overnight. The MVK insert in the resulting plasmid MCM382 was sequenced (FIGS. 47A-47C).

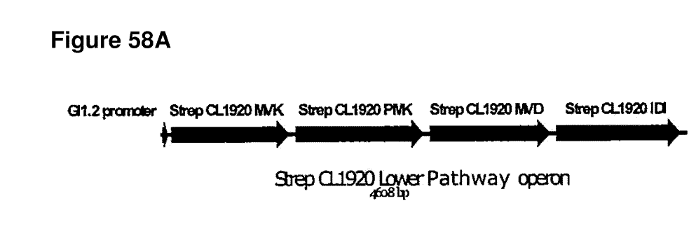

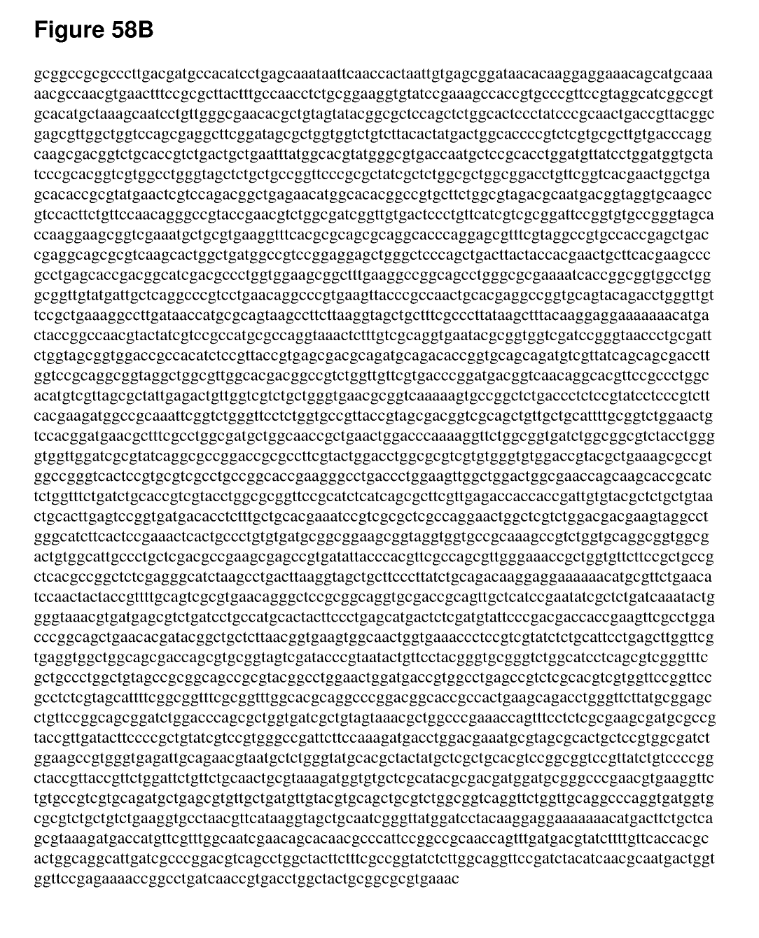

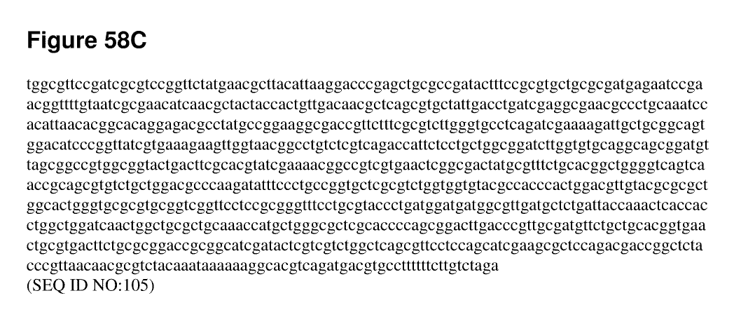

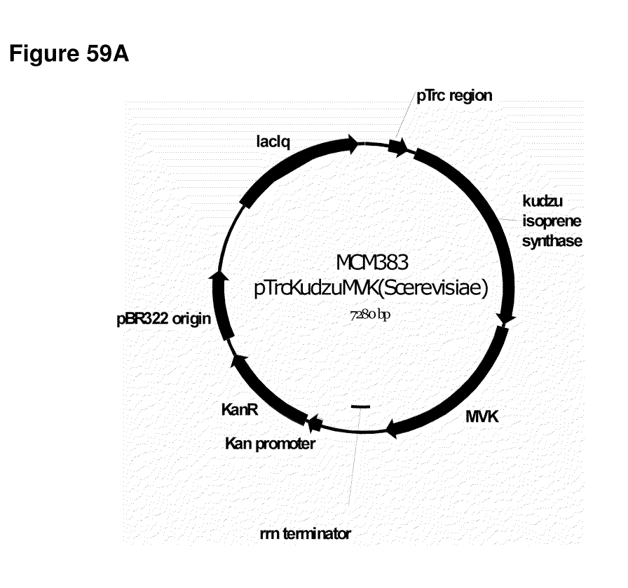

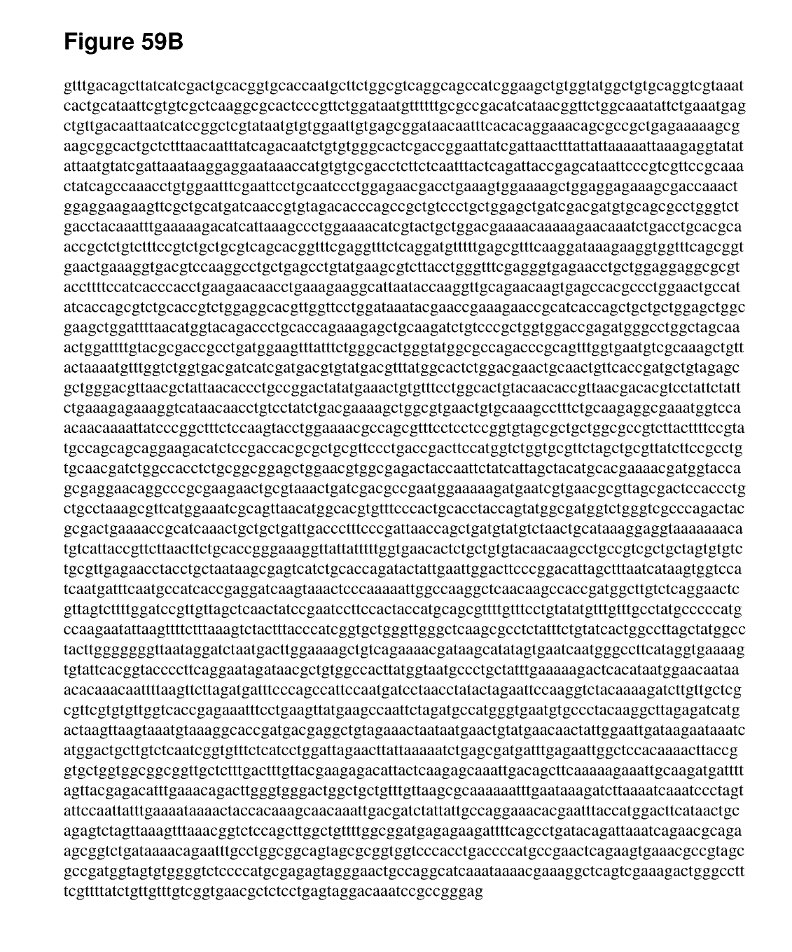

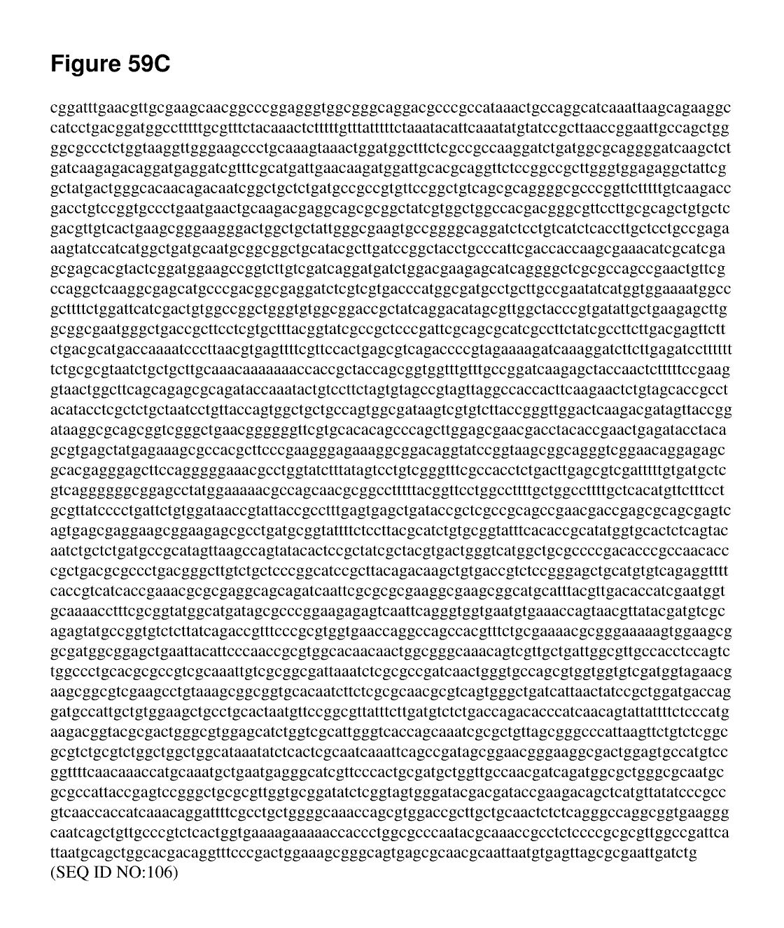

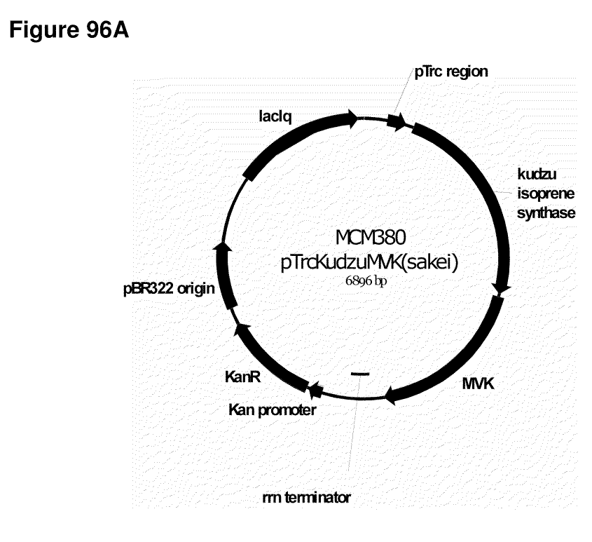

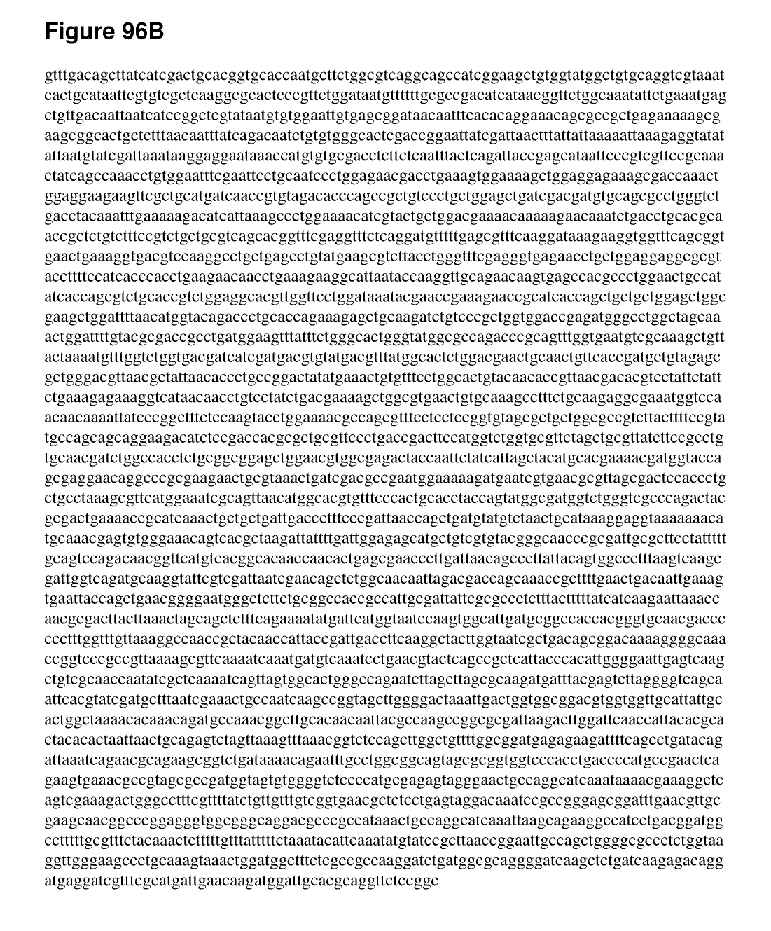

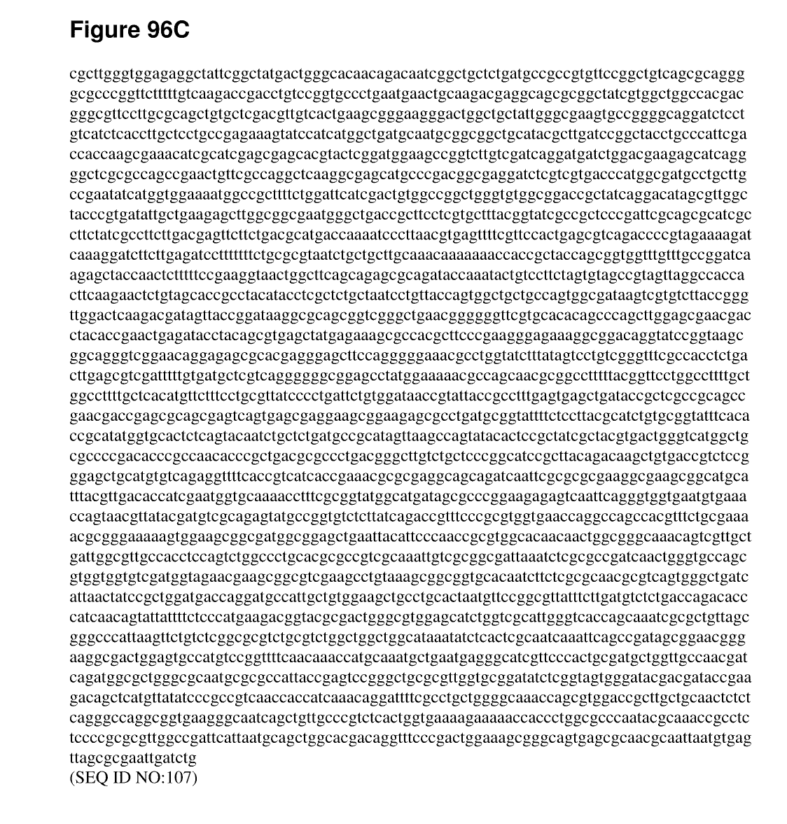

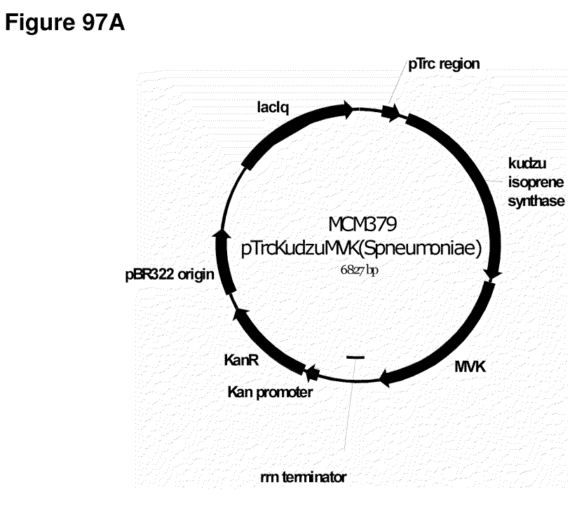

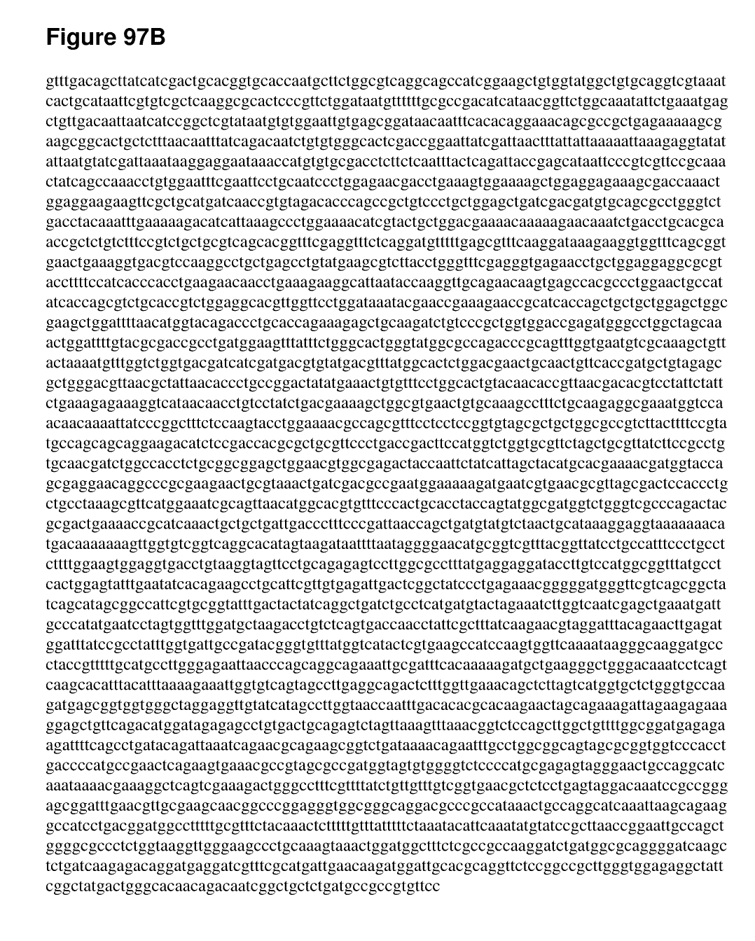

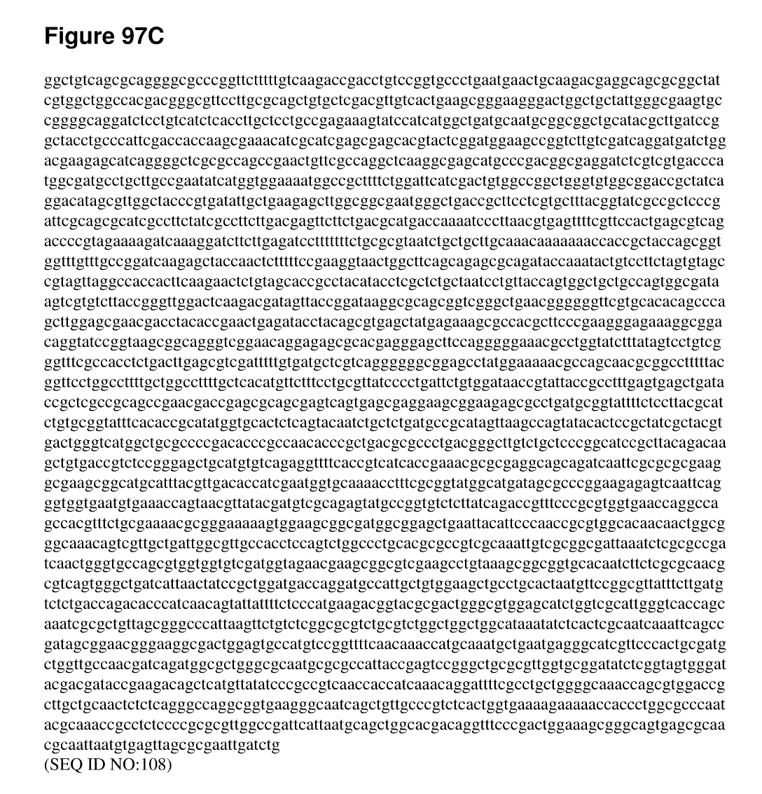

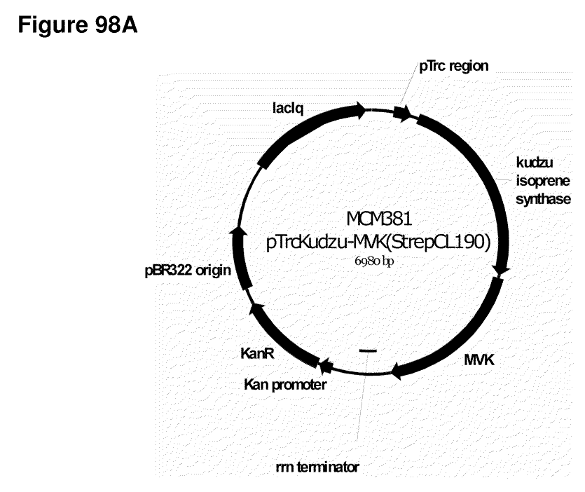

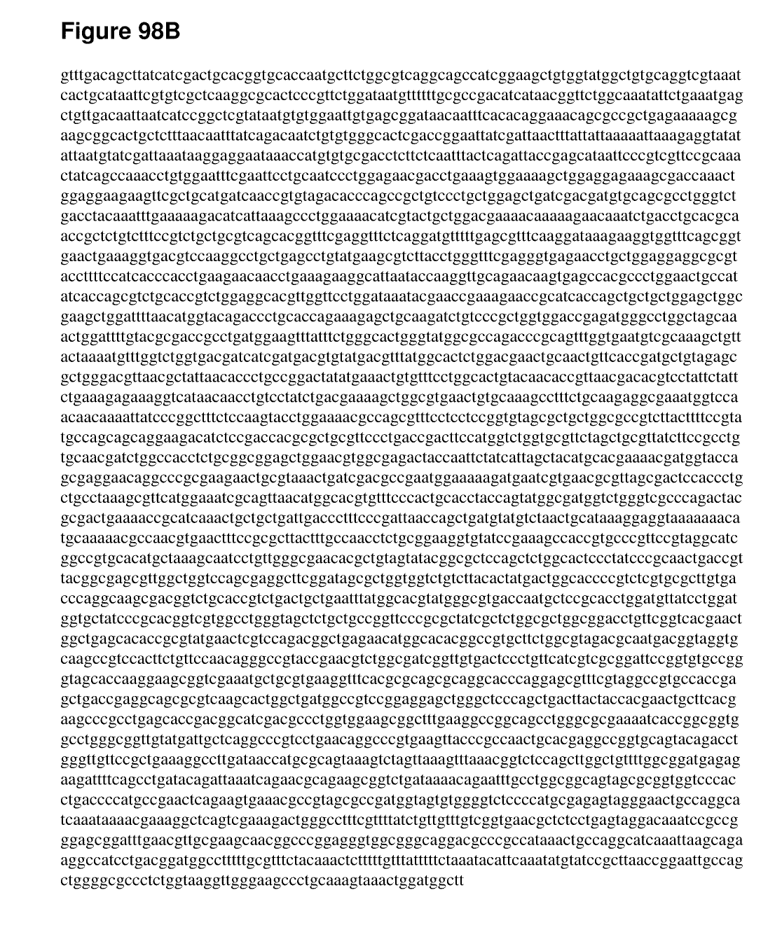

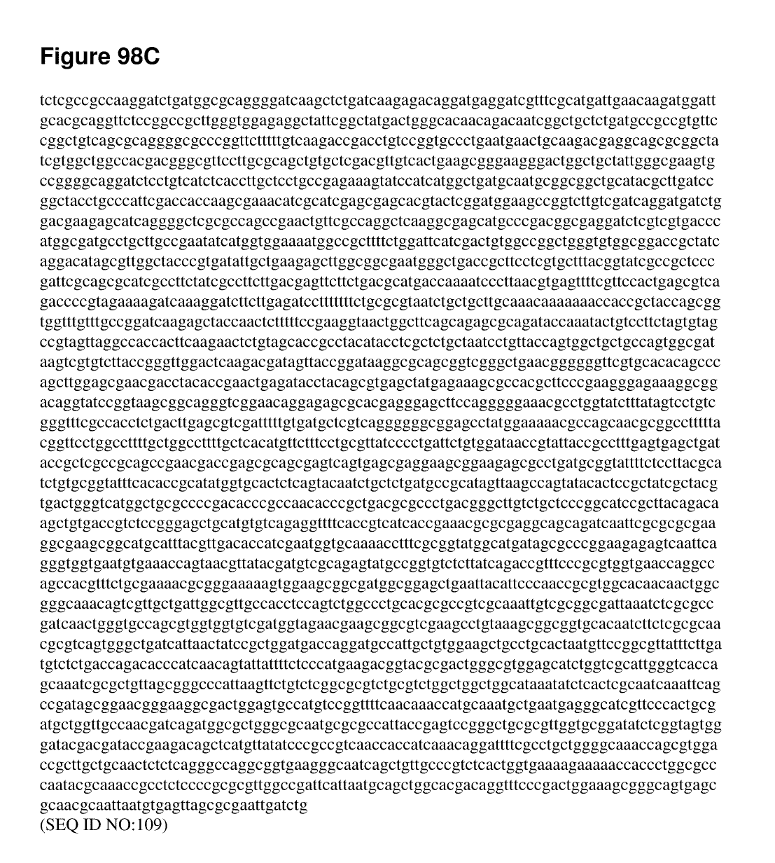

Using the method described above for plasmid MCM382, pTrcKudzu-MVK(mazei), four additional plasmids were constructed with MVK genes from different source organisms (Table 5 and FIGS. 58A-58C, 59A-59C, 96A-96C, 97A-97C, and 98A-98C).

TABLE 5
Plasmids encoding MVK from different source organisms.

| | | Forward | Reverse | Final | MVK Protein |
|---|---|---|---|---|---|
| Source Organism | PCR Template | Primer | Primer | Plasmid | Accession |
| Streptococcus | pDW02 | MCM166 | MCM167 | MCM379 | |
| penumoniae | | | | | |
| Lactobacillus | pDW01 | MCM168 | MCM169 | MCM380 | |
| sakei | | | | | |
| Streptomyces | Streptomyces | MCM164 | MCM176 | MCM381 | BAB07790.1 |
| CL190 | CL190 Lower | | | | |
| | Pathway operon | | | | |
| Saccharomyces | pTrcKK | MCM170 | MCM171 | MCM383 | |
| cerevisiae | (described herein) | | | | |

### II. Creation of Strains Overexpressing Mevalonate Kinase and Isoprene Synthase.

Plasmid MCM382 was transformed into MCM331 cells (which contain chromosomal construct gi1.2KKDyI encoding S. cerevisiae mevalonate kinase, mevalonate phosphate kinase, mevalonate pyrophosphate decarboxylase, and IPP isomerase) that had been grown to midlog in LB medium and washed three times in iced, sterile water. 1 uL of DNA was added to 50 uL of cell suspension, and this mixture was electroporated in a 2 mm cuvette at 2.5 volts, 25 uFd followed immediately by recovery in 500 uL LB medium for one hour at 37° C. Transformant was selected on LA/kan50 and named MCM391. Plasmid MCM82 was introduced into this strain by the same electroporation protocol followed by selection on LA/kan50/spec50. The resulting strain MCM401 contains a cmp-marked chromosomal construct gi1.2KKDyI, kan-marked plasmid MCM382, and spec-marked plasmid MCM82 (which is pCL PtrcUpperPathway encoding E. faecalis mvaE and mvaS).

Production strains analogous to MCM401 were generated for each of the four plasmids detailed in Table 5 using the methods described above for MCM401. MCM331 was transformed with plasmid MCM379, 380, 381, or 383, and then selected on LA+kan50. The resulting strains were transformed with MCM82 and selected on LA+kan50+spec50.

TABLE 6
Strains overexpressing mevalonate kinase and isoprene synthase

| | | | Strain MCM331 |
|---|---|---|---|
| | | | transformed with |
| | | Strain MCM331 | pTrcKudzuMVK then |
| | Plasmid | transformed with | transformed with |
| MVK Source | pTrcKudzu-MVK | pTrcKudzuMVK | MCM82 |
| Streptococcus pneumoniae | MCM379 | MCM388 | MCM398 |
| Lactobacillus sakei | MCM380 | MCM389 | MCM399 |
| Streptomyces CL190 | MCM381 | MCM390 | MCM400 |
| Methanosarcina mazei | MCM382 | MCM391 | MCM401 |
| Saccharomyces cerevisiae | MCM383 | MCM392 | MCM402 |
| | | | Strain MCM333 |
| | | | transformed with |
| | | Strain MCM333 | pTrcKudzuMVK then |
| | Plasmid | transformed with | transformed with |
| MVK Source | pTrcKudzu-MVK | pTrcKudzuMVK | MCM82 |
| Streptococcus pneumoniae | MCM379 | MCM393 | MCM403 |
| Lactobacillus sakei | MCM380 | MCM394 | MCM404 |
| Streptomyces CL190 | MCM381 | MCM395 | |
| Methanosarcina mazei | MCM382 | MCM396 | MCM406 |
| Saccharomyces cerevisiae | MCM383 | MCM397 | MCM407 |

Additional strain information is provided below.
MCM382: E. coli BL21 (lambdaDE3) pTrcKudzuMVK(M. mazei)GI1.2KKDyI
MCM391: MCM331pTrcKudzuMVK(M. mazei)
MCM401: MCM331pTrcKudzuMVK(M. mazei)pCLPtrcUpperpathway
MCM396: MCM333pTrcKudzuMVK(M. mazei)
MCM406:: MCM333pTrcKudzuMVK(M. mazei)pCLPtrcUpperpathway
   III. Construction of Plasmid MCM376—MVK from M. mazei Archaeal Lower in pET200D.

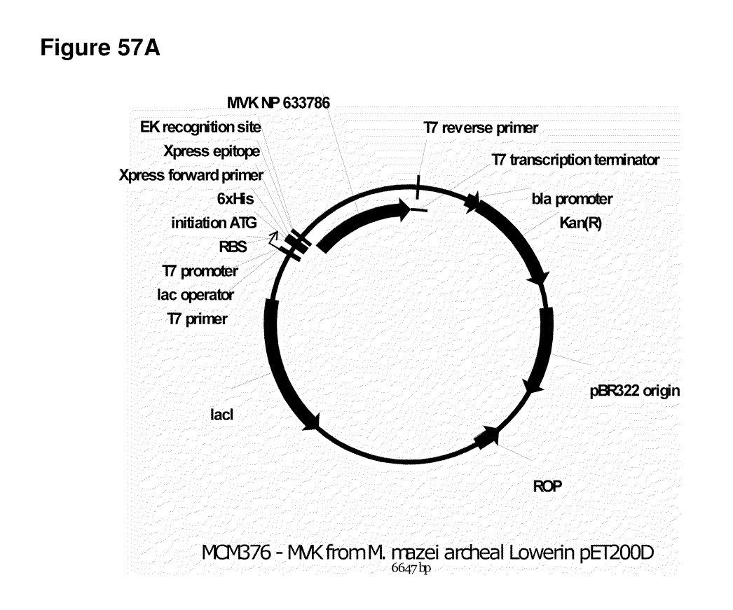

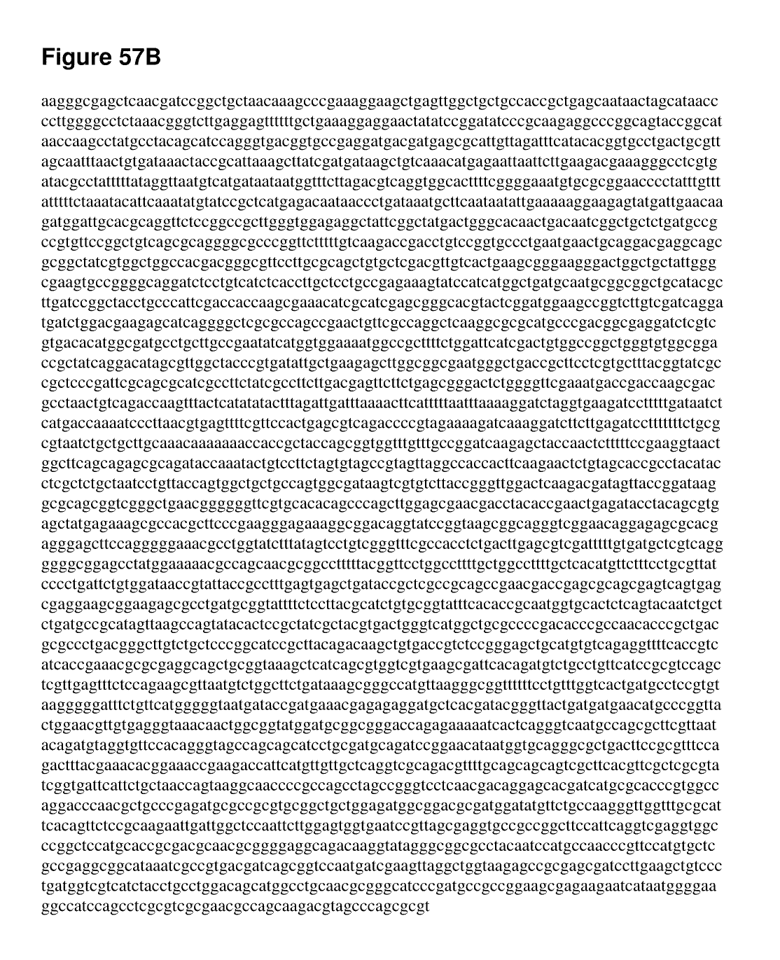

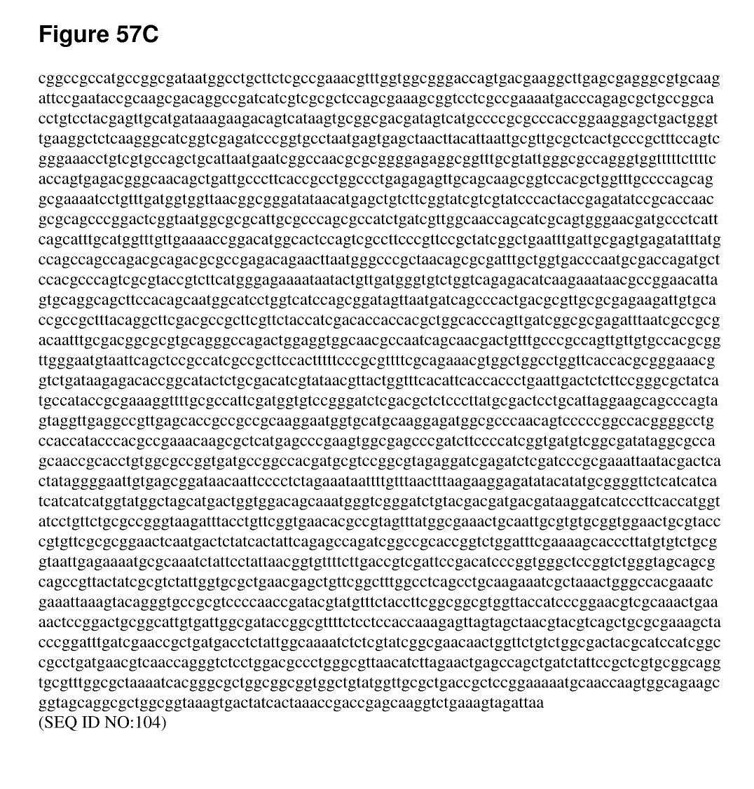

The MVK ORF from the M. mazei archaeal Lower Pathway operon (FIGS. 46A-46C) was PCR amplified using primers MCM161 and MCM162 (Table 4) using the Invitrogen Platinum HiFi PCR mix. 45 uL of PCR mix was combined with 1 uL template, 1 uL of each primer at 10 uM, and 2 uL water. The reaction was cycled as follows: 94° C. for 2:00; 30 cycles of 94° C. for 0:30, 55° C. for 0:30. and 68° C. for 1:15; and then 72° C. for 7:00, and 4° C. until cool. 3 uL of this PCR reaction was ligated to Invitrogen pET200D plasmid according to the manufacturer's protocol. 3 uL of this ligation was introduced into Invitrogen TOP10 cells, and transformants were selected on LA/kan50. A plasmid from a transformant was isolated and the insert sequenced, resulting in MCM376 (FIGS. 57A-57C).

### IV. Construction of MCM420 Expressing Streptomyces CL190 MVK

The Streptomyces CL190 MVK was cloned into pET200D as described above for plasmid MCM376 (Table 7).

V. Construction of pDu5 Expressing S. cerevisiae MVK

The S. cerevisiae MVK was cloned into pET16b from Invitrogen as follows (Table 7). The MVK enzyme from S. cerevisiae was PCR amplified with Hg-MVK-F2-NdeI and Hg-MVK-R2-NdeI primers using Stratagene Pfu UltraII Fusion DNA Polymerase Kit according to manufacturer's protocol, and pMVK1 (described herein) as the template DNA. The following cycle parameter was used for the reaction (95° C. for 2 minutes, 29cycles (95° C. for 20 seconds, 55° C. for 20 seconds, 72° C. for 21 seconds), 72° C. for 3 minutes, and 4° C. until cool) using an Eppendorf Mastercycler Gradient Machine).

As a result, a 1.352 kb MVK PCR fragment was obtained and was gel purified using Qiagen's gel purification kit. The purified PCR product was digested with NdeI restriction enzyme. The digested DNA was purified over Qiagen PCR column. 5 uL of purified PCR product was ligated to 1 uL of pET-16b vector that was previously digested with NdeI and then treated with SAP (Shrimp Alkaline Phosphatase). A New England BioLab (NEB) T4 ligase kit was used for ligation at approximately 16° C. overnight according to manufacturer's protocol.

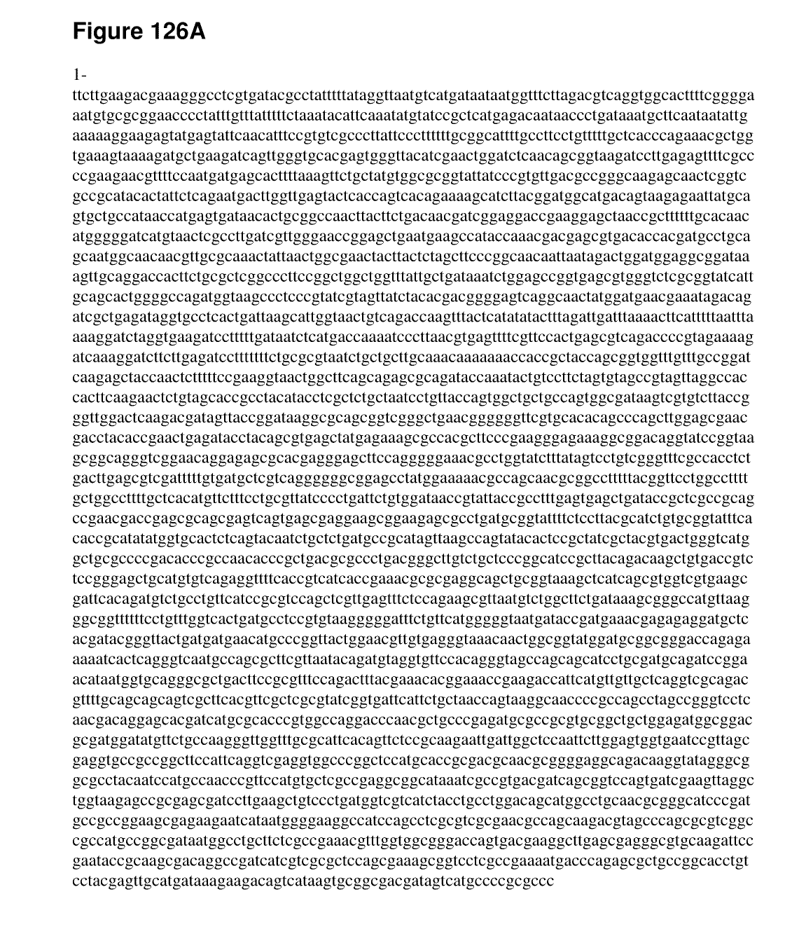

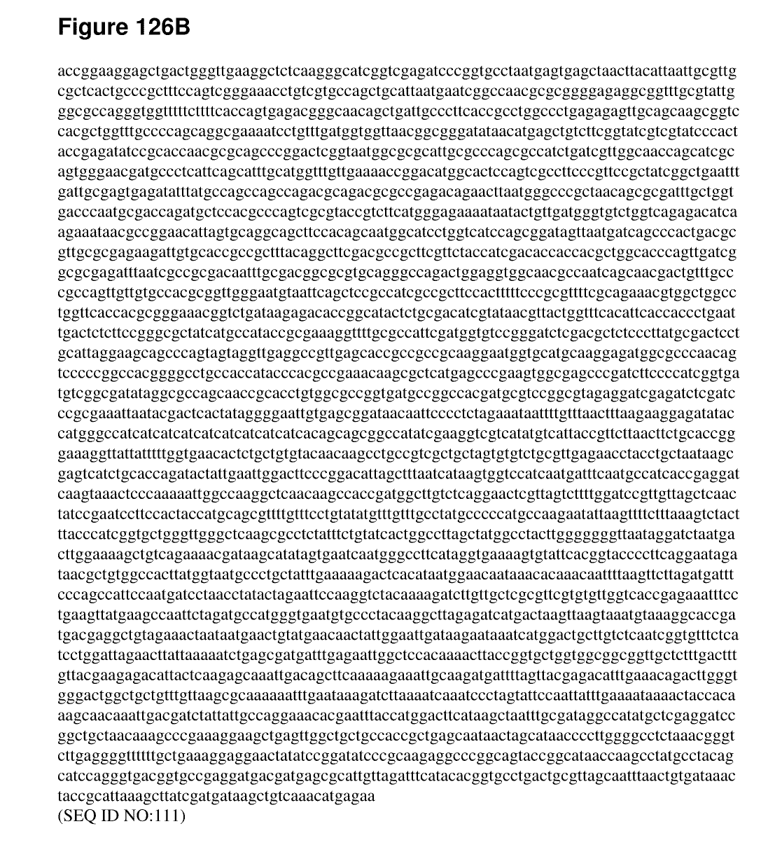

5 uL of overnight ligation mixture was transformed into Invitrogen TOP10 cells. The transformation was carried on ice for a 30 minute incubation followed by a 30 second heat shock at approximately 42° C. and a 1 hour recovery in 1 ml LB at approximately 37° C. The transformation was selected on LA/Carb50 incubated at approximately 37° C. overnight. Plasmids from transformants were isolated and the insert sequenced with T7 promoter and T7 terminator using Quintara Bio Sequencing Service. The resulting plasmid for S. cerevisiae MVK in pET-16b vector is called pDu5 (FIGS. 126A and 126B).

Once the sequence is verified, 1 ul of plasmid (pDu5) is then transformed into BL21 pLysS host strain. Transformants are selected on LA/Carb50 plates and incubated at approximately 37° C. The resulting expression strain is called MD08-MVK.

TABLE 7
Plasmids and Strains overexpressing mevalonate kinase

| | | | | | Expression |
|---|---|---|---|---|---|
| | Template | For. Primer | Rev. Primer | Plasmid | Strain |
| S. cerevisiae | pMVK1 | Hg-MVK-F2-NdeI | Hg-MVK-R2-NdeI | pDu5 | MD08-MVK |
| Streptomyces | Streptomyces | MCM159 | MCM160 | MCM420 | MCM429 |
| CL190 | CL190 Lower | | | | |
| | Pathway operon | | | | |

### V. Creation of Expression Strain MCM378.

Plasmid MCM376 was transformed into Invitrogen BL21 Star (DE3) cells according to the manufacturer's protocol. Transformant MCM378 was selected on LA/kan50. Additional strains were created using the same protocol and are listed in the Table 7. Invitrogen OneShot BL21(DE3) pLysS transformed with the indicatd plasmid and selected on LA and carb50 cmp35 (for MD08-MVK) or selected on LA and kan50 cmp35 (for MCM429) were used.

VI. Construction of Plasmid pCLPtrcUpperPathway HGS2

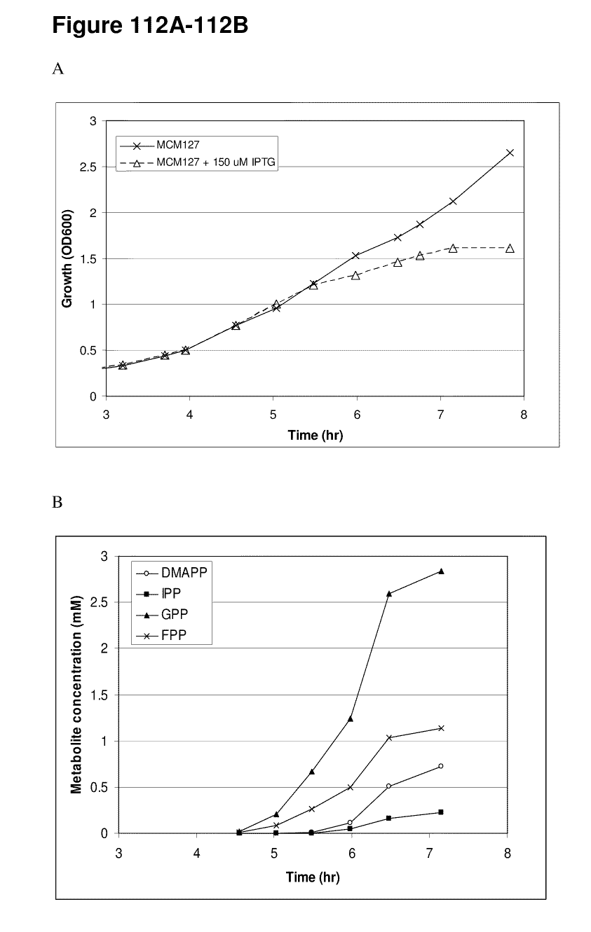

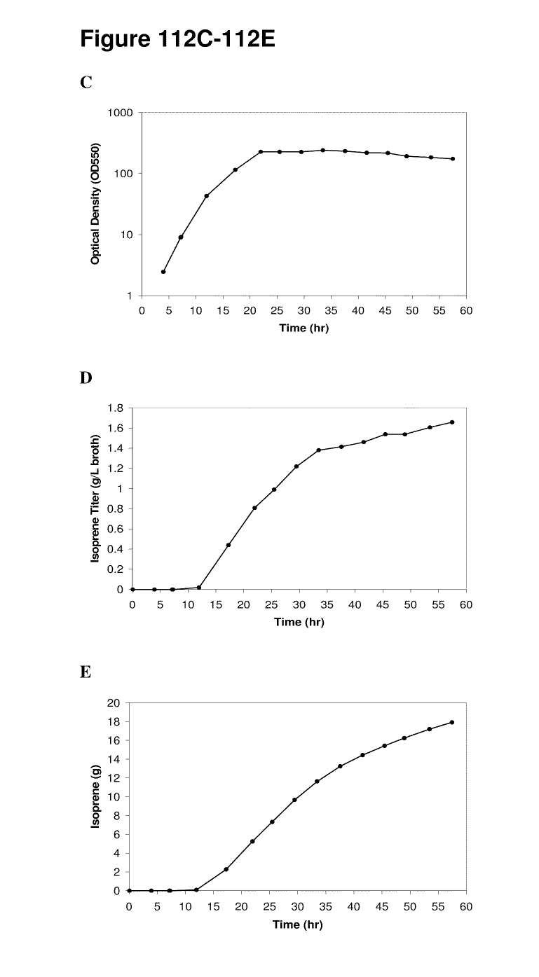

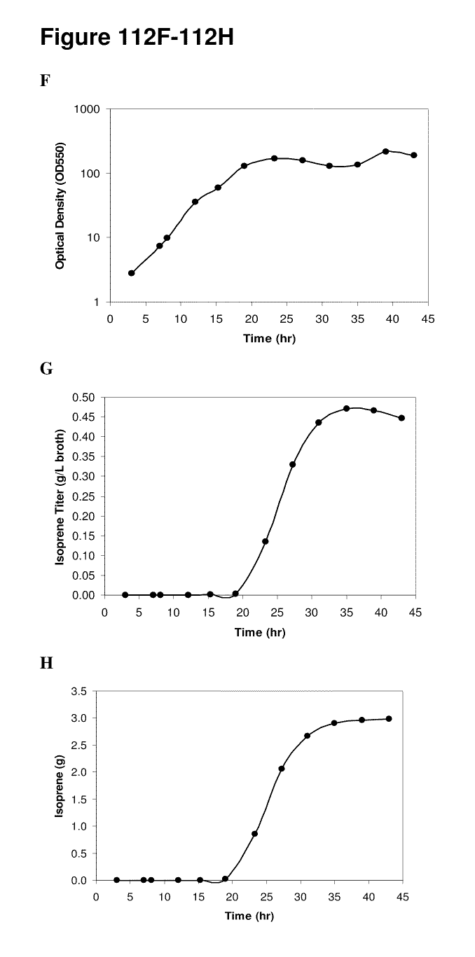

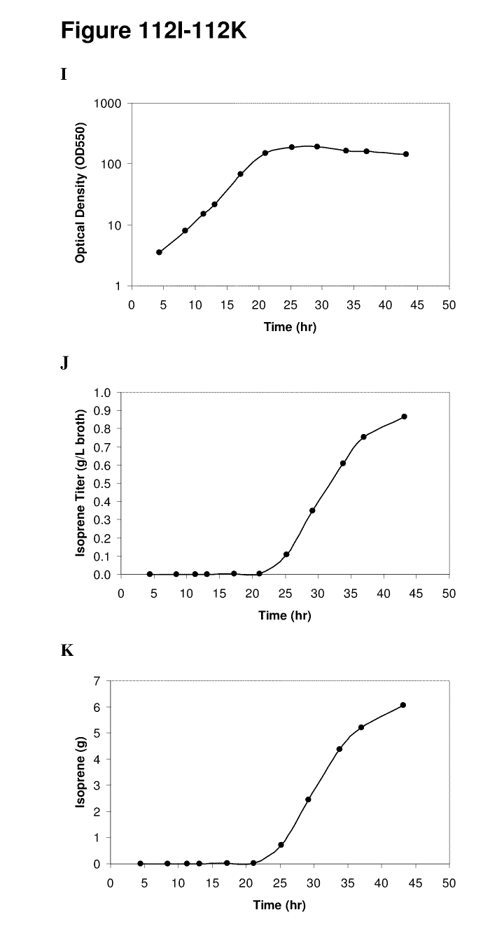

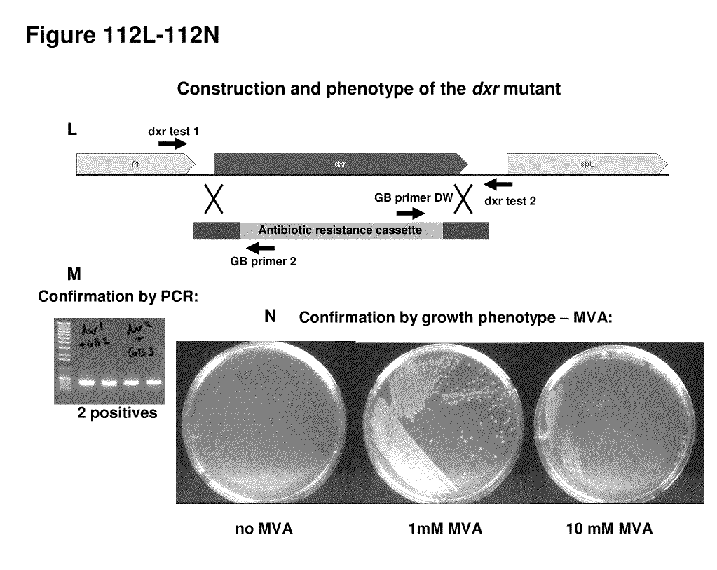

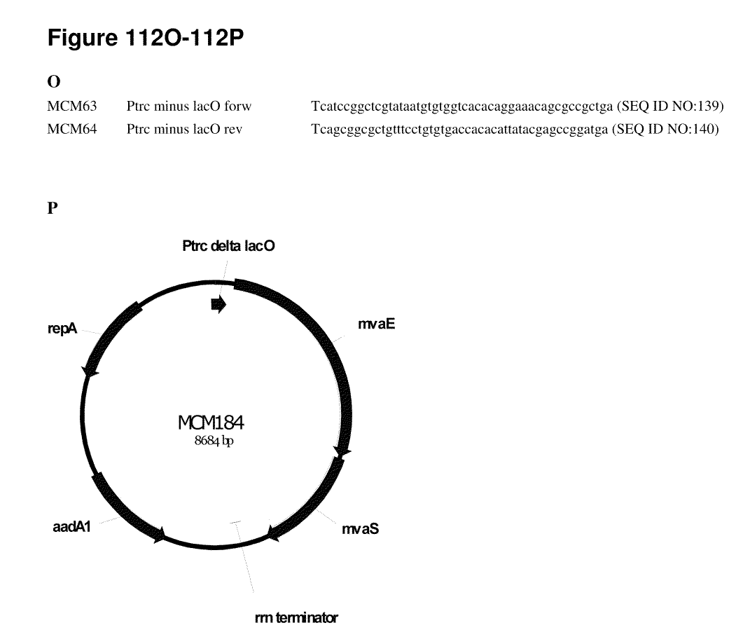

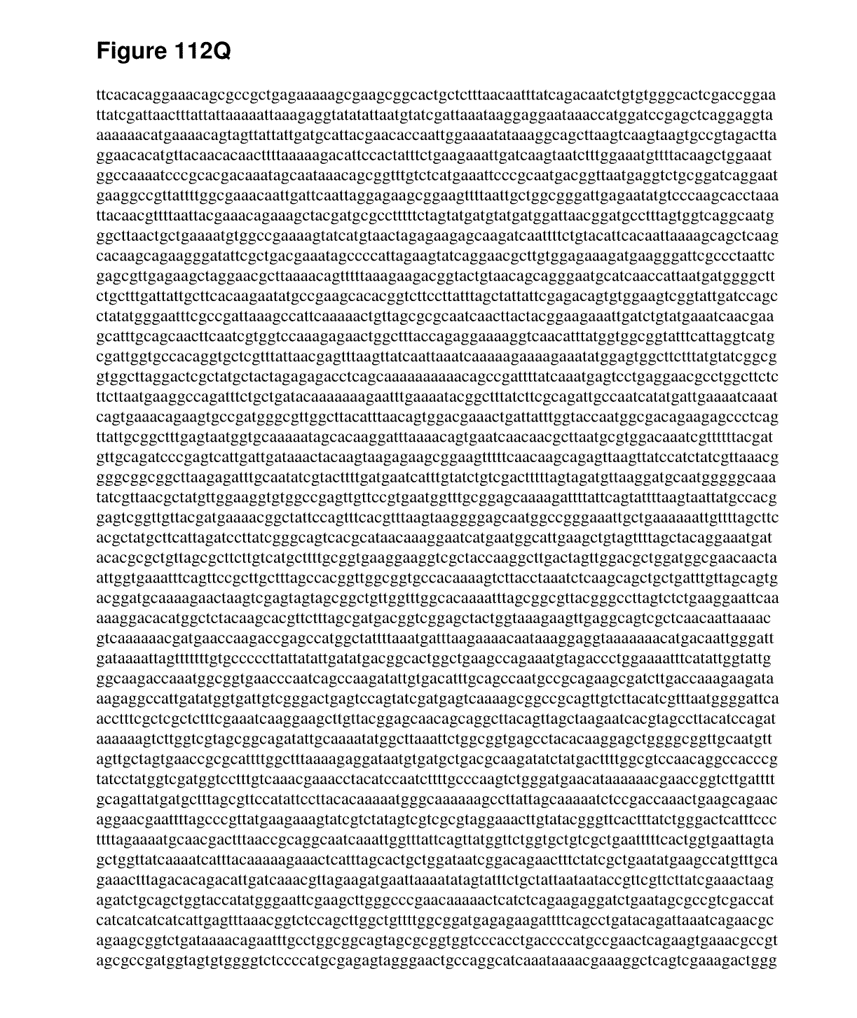

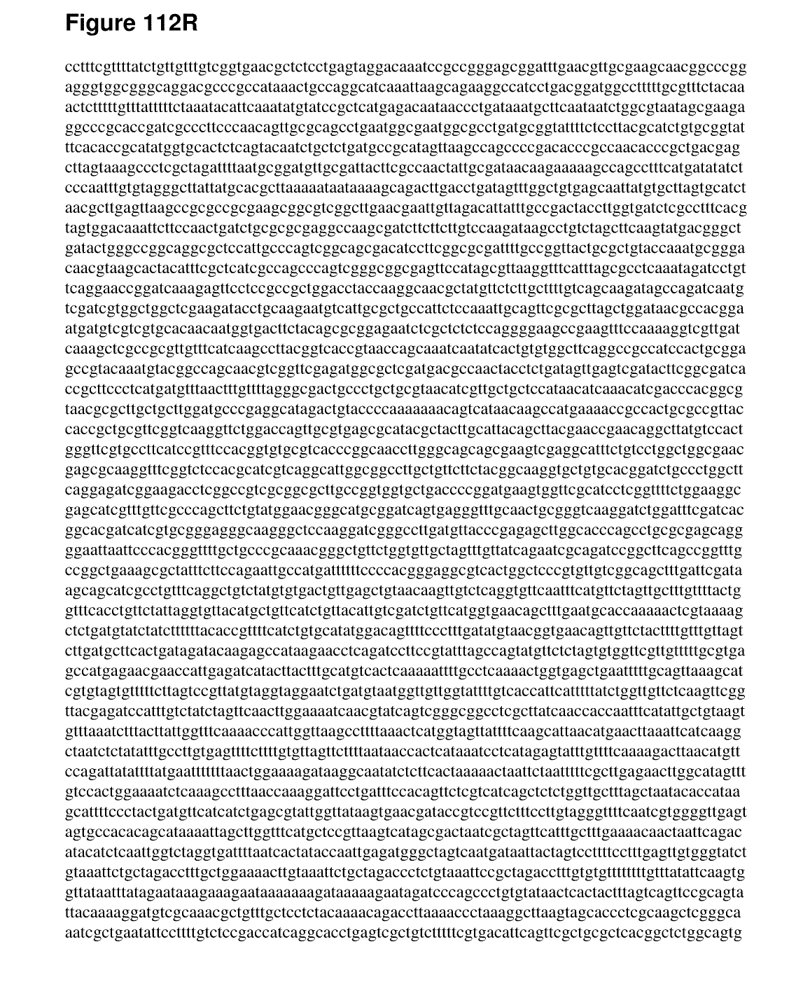

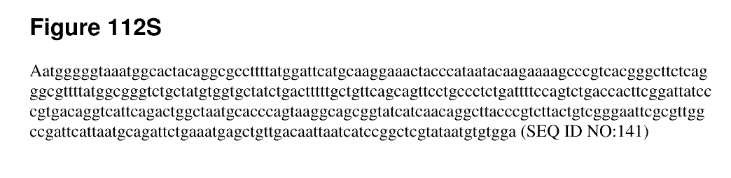

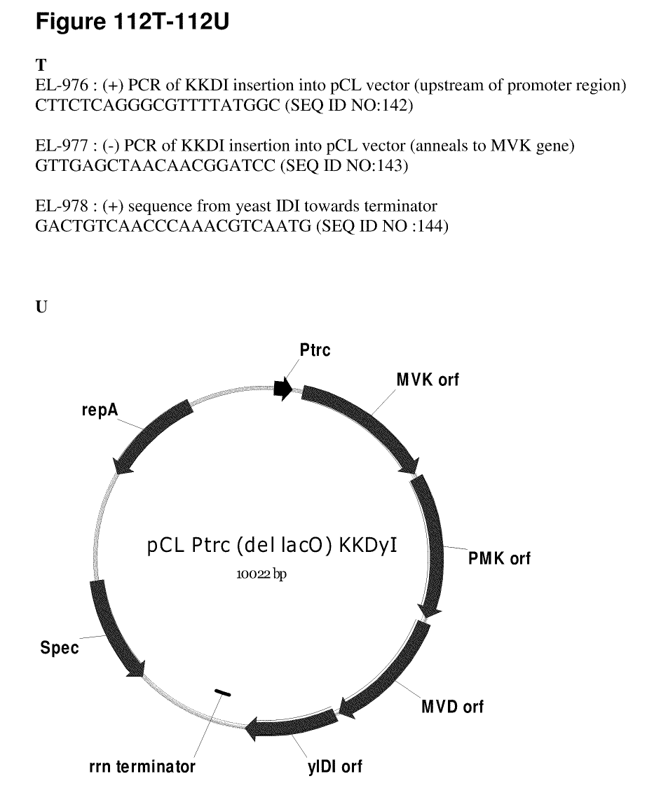

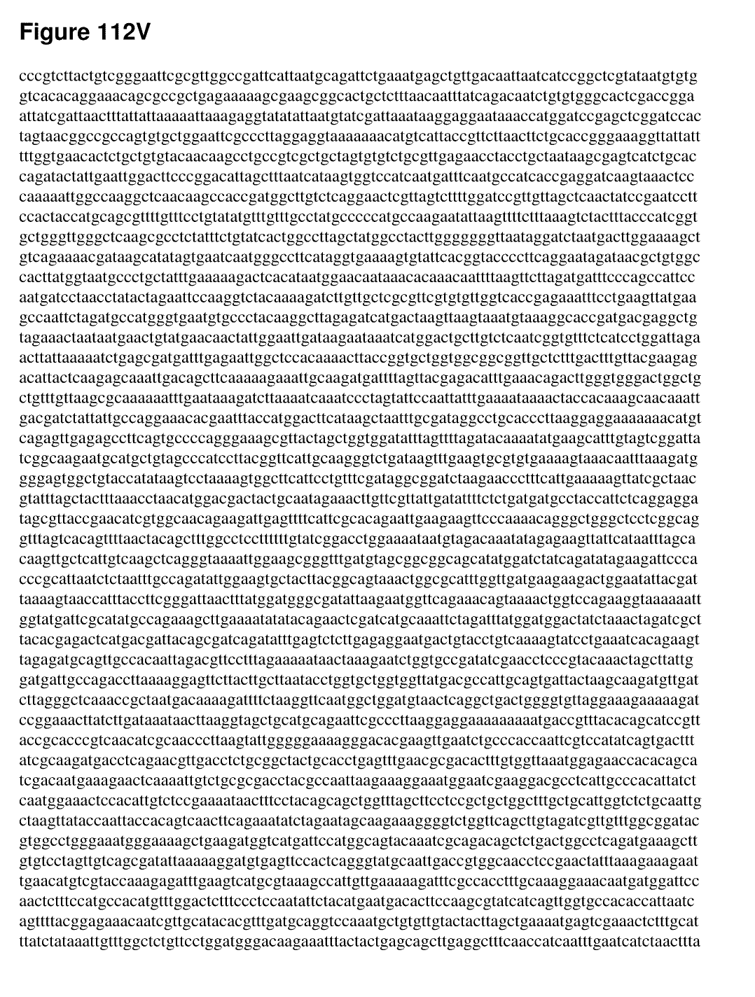

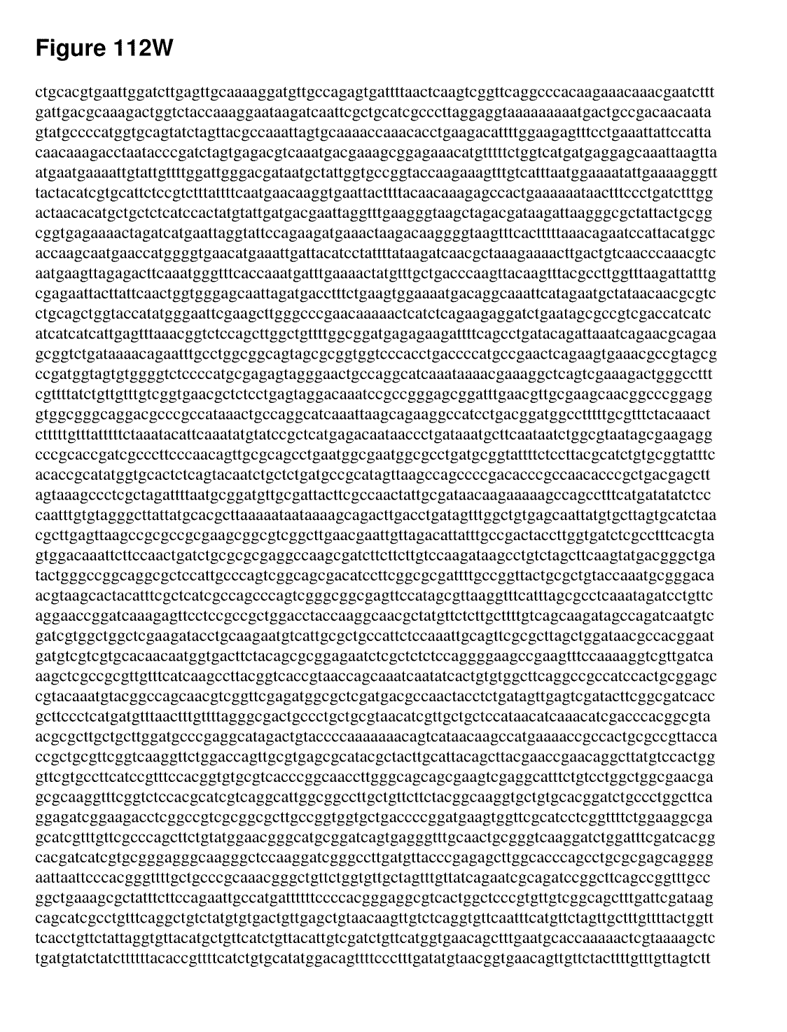

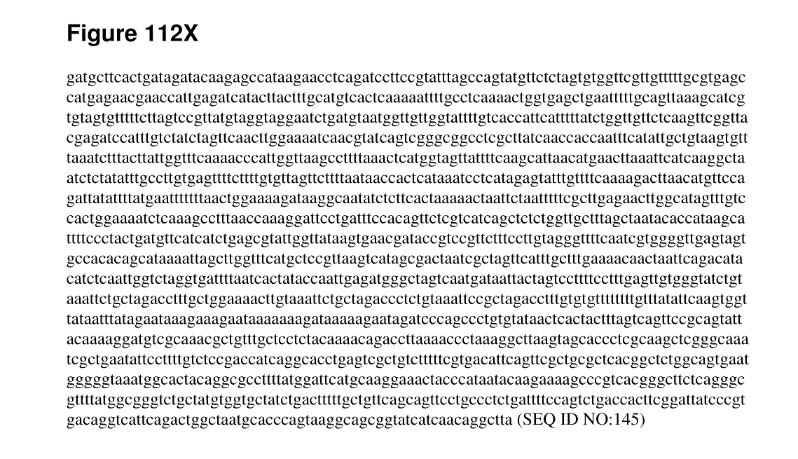

The gene encoding isoprene synthase from Pueraria lobata was PCR-amplified using primers NsiI-RBS-HGS F (cttgATGCATCCTGCATTCGCCCTTAGGAGG, SEQ ID NO:115) and pTrcR (CCAGGCAAATTCTGTTTTATCAG, SEQ ID NO:116), and pTrcKKDyIkIS (MCM118) as a template. The resulting PCR product was restriction-digested with NsiI and PstI and gel-purified using the Qiagen QIAquick Gel Extraction kit using standard methods. MCM82 (pCL PtrcUpperPathway) was restriction-digested with PstI and dephosphorylated using rAPid alkaline phosphatase (Roche). These DNA pieces were ligated together using T4 ligase and the ligation reaction was transformed in E. coli Top10 electrocompetent cells (Invitrogen). Plasmid was prepared from six clones using the Qiagen QiaPrep Spin MiniPrep kit. The plasmids were digested with restriction enzymes EcoRV and MluI, and a clone in which the insert had the right orientation (i.e., gene oriented in the same way as the pTrc promoter) was identified. The resulting plasmid pCLPtrcUpperPathwayHGS2 (FIGS. 112A-112D) was found to produce isoprene in E. coli Top10, using a headspace assay described herein, thus validating the functionality of the expression construct.

TABLE 4
Oligonucleotides

| | | |
|---|---|---|
| | Hg-MVK-F2-NdeI | cagcagcagCATATGtcattaccgttcttaacttc (SEQ ID NO: 117) |
| | Hg-MVK-R2-NdeI | cagcagcagCATATGgcctatcgcaaattagcttatg (SEQ ID NO: 118) |
| MCM159 | Strep CL190 MVK for | CACCATGCAAAAACGCCAACGTGA (SEQ ID NO: 119) |
| MCM160 | Strep CL190 MVK rev | TTACTGCGCATGGTTATCAAGGC (SEQ ID NO: 120) |
| MCM161 | M. mazei MVK for | CACCATGGTATCCTGTTCTGCG (SEQ ID NO: 121) |
| MCM162 | M. mazei MVK rev | TTAATCTACTTTCAGACCTTGC (SEQ ID NO: 122) |
| MCM164 | Strep CL190 MVK for w/ | gcgaacgATGCATaaaggaggtaaaaaaacATGCAAAAACGCCAACGTGA (SEQ ID NO: 123) |
| | RBS | |
| MCM165 | M. mazei MVK for w/ RBS | gcgaacgATGCATaaaggaggtaaaaaaacATGGTATCCTGTTCTGCGCCGGGTAAGATTTAC |
| | | CTG (SEQ ID NO: 124) |
| MCM166 | S. pneumoniae MVK for w/ | gcgaacgATGCATaaaggaggtaaaaaaacATGACAAAAAAAGTTGGTGTCGGT (SEQ ID |
| | RBS | NO: 125) |
| MCM167 | S. pneumoniae MVK rev | gggcccgtttaaactttaactagactCTGCAGTCACAGGCTCTCTATCCATGTCTGAA (SEQ ID |
| | | NO: 126) |
| MCM168 | L. sakei MVK for w/ RBS | gcgaacgATGCATaaaggaggtaaaaaaacATGCAAACGAGTGTGGGAAACA (SEQ ID |
| | | NO: 127) |
| MCM169 | L. sakei MVK rev | gggcccgtttaaactttaactagactCTGCAGTTAATTAGTGTGTAGTGCGTGTAATGGTTG |
| | | (SEQ ID NO: 128) |
| MCM170 | S. cerevisiae MVK for w/ | gcgaacgATGCATaaaggaggtaaaaaaacATGTCATTACCGTTCTTAACTTCTGCA (SEQ ID |
| | RBS | NO: 129) |
| MCM171 | S. cerevisiae MVK rev | gggcccgtttaaactttaactagactCTGCAGTTATGAAGTCCATGGTAAATTCGTGT (SEQ ID |
| | | NO: 130) |
| MCM176 | Strep CL190 MVK rev Pst | gggcccgtttaaactttaactagactTTACTGCGCATGGTTATCAAGGC (SEQ ID NO: 131) |
| MCM177 | M. mazei MVK rev Pst | gggcccgtttaaactttaactagactTTAATCTACTTTCAGACCTTGC (SEQ ID NO: 132) |

### Example 2

### Production of Isoprene by E. coli Expressing the Upper Mevalonic Acid (MVA) Pathway, the Integrated Lower MVA Pathway (gi1.2KKDyI), Mevalonate Kinase from M. mazei, and Isoprene Synthase from Kudzu and Grown in Fed-Batch Culture at the 20 mL Batch Scale

### Medium Recipe (Per Liter Fermentation Medium):

Each liter of fermentation medium contained K₂HPO₄ 13.6 g, KH₂PO₄ 13.6 g, MgSO₄* 7H₂O 2 g, citric acid monohydrate 2 g, ferric ammonium citrate 0.3 g, (NH₄)₂SO₄ 3.2 g, yeast extract 1 g, and 1000× Trace Metal Solution 1 ml. All of the components were added together and dissolved in diH₂O. The pH was adjusted to 6.8 with ammonium hydroxide (30%) and brought to volume. Media was filter sterilized with a 0.22 micron filter. Glucose 2.5 g and antibiotics were added after sterilization and pH adjustment.

### 1000× Trace Metal Solution:

1000× Trace Metal Solution contained citric Acids*H₂O 40 g, MnSO₄*H₂O 30 g, NaCl 10 g, FeSO₄*7H₂O 1 g, CoCl₂*6H₂O 1 g, ZnSO₄*7H₂O 1 g, CuSO₄*5H₂O 100 mg, H₃BO₃ 100 mg, and NaMoO₄*2H₂O 100 mg. Each component was dissolved one at a time in DI H₂O, pH to 3.0 with HCl/NaOH, then brought to volume and filter sterilized with a 0.22 micron filter.

### Strains:

MCM343 cells are BL21 (DE3) E. coli cells containing the upper mevalonic acid (MVA) pathway (pCL Upper), the integrated lower MVA pathway (gi1.2KKDyI), and isoprene synthase from Kudzu (pTrcKudzu). The S. cerevisiae MVK gene is present only as one copy on the chromosome of the MCM343 cells and is controlled by a weak promoter. The expression level of isoprene synthase may not be limiting in the MCM343 cells. The isoprene synthase gene has the same plasmid backbone and promoter as in the MCM401 cells.

MCM401 cells are BL21 (DE3) E. coli cells containing the upper mevalonic acid (MVA) pathway (pCL Upper), the integrated lower MVA pathway (gi1.2KKDyI), and high expression of mevalonate kinase from M. mazei and isoprene synthase from Kudzu (pTrcKudzuMVK(M. mazei)). The M. mazei MVK gene is present in multiple copies on a plasmid in the MCM401 cells (˜30-50 copies/cell) and is under a stronger promoter than the S. cerevisiae MVK gene. Based on this information, the MVK protein level in the MCM401 cells is expected to be at least about 30 to 50 fold higher than the level in the MCM343 cells. The expression level of isoprene synthase may not be limiting in the MCM401 cells. The isoprene synthase gene shares the same plasmid backbone and promoter as the MCM343 cells. In addition, the amount of isoprene synthase made is higher in the MCM401 cells, and the protein level of the isoprene synthase was not dependent upon the inhibition of MVK.

Isoprene production was analyzed by growing the strains in 100 mL bioreactors with a 20 mL working volume at a temperature of 30° C. An inoculum of E. coli strain taken from a frozen vial was streaked onto an LB broth agar plate (with antibiotics) and incubated at 30° C. A single colony was inoculated into media and grown overnight. The bacteria were diluted into 20 mL of media to reach an optical density of 0.05 measured at 550 nm. The 100 mL bioreactors were sealed, and air was pumped through at a rate of 8 mL/min. Adequate agitation of the media was obtained by stirring at 600 rpm using magnetic stir bars. The off-gas from the bioreactors was analyzed using an on-line Hiden HPR-20 mass spectrometer. Masses corresponding to isoprene, CO₂, and other gasses naturally occurring in air were monitored. Accumulated isoprene and CO₂ production were calculated by summing the concentration (in percent) of the respective gasses over time. Atmospheric CO₂ was subtracted from the total in order to estimate the CO₂ released due to metabolic activity.

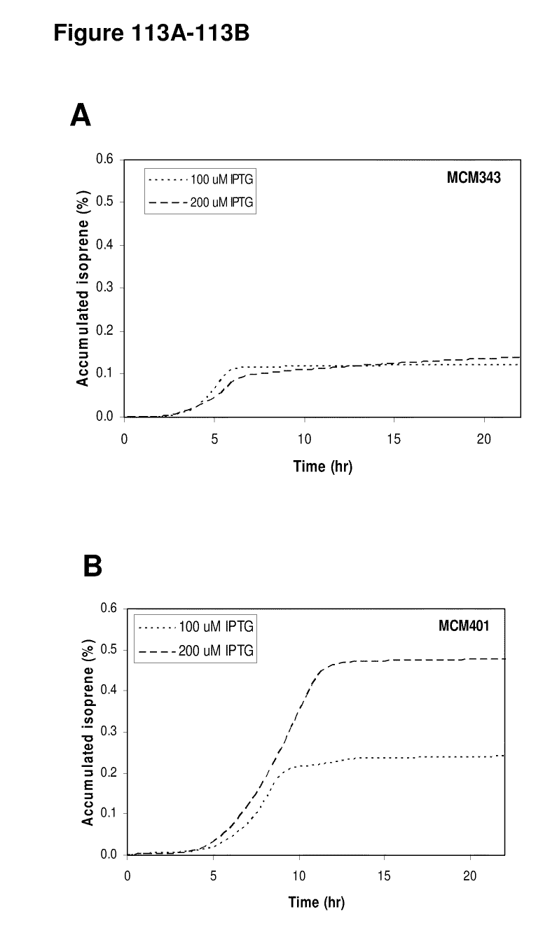

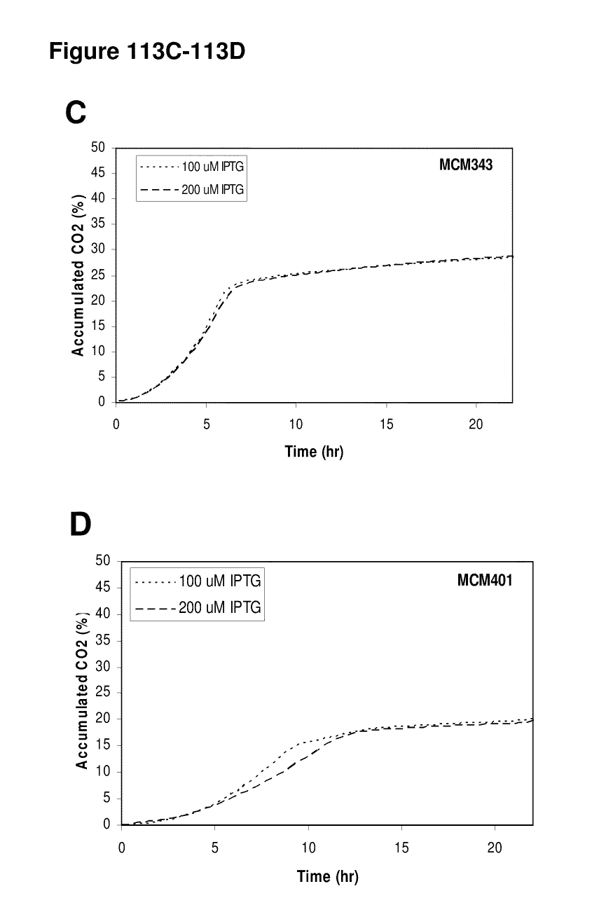

Isoprene production from a strain expressing the full mevalonic acid pathway and Kudzu isoprene synthase (MCM343) was compared to a strain that in addition over-expressed MVK from M. mazei and Kudzu isoprene synthase (MCM401) in 100 mL bioreactors. The bacteria were grown under identical conditions in defined media with glucose as carbon source. Induction of isoprene production was achieved by adding isopropyl-beta-D-1-thiogalactopyranoside (IPTG) to a final concentration of either 100 uM or 200 uM. Off-gas measurements revealed that the strain over-expressing both MVK and isoprene synthase (MCM401) produced significantly more isoprene compared to the strain expressing only the mevalonic acid pathway and Kudzu isoprene synthase (MCM343) as shown in FIGS. 113A-113D. At 100 uM induction, the MCM401 strain produced 2-fold more isoprene compared to the MCM343 strain. At 200 uM IPTG induction, the MCM401 strain produced 3.4-fold more isoprene when compared to the MCM343 strain. Analysis of CO₂ in the off-gas from the bioreactors, which is a measure of metabolic activity, indicates that metabolic activity was independent from IPTG induction and isoprene production.

### Example 3

### Production of Isoprene by E. coli Expressing the Upper Mevalonic Acid (MVA) Pathway, the Integrated Lower MVA Pathway (gi1.2KKDyI), Mevalonate Kinase from M. mazei, and Isoprene Synthase from Kudzu and Grown in Fed-Batch Culture at the 15-L Scale

### Medium Recipe (Per Liter Fermentation Medium):

Each liter of fermentation medium contained K₂HPO₄ 7.5 g, MgSO₄*7H₂O 2 g, citric acid monohydrate 2 g, ferric ammonium citrate 0.3 g, yeast extract 0.5 g, and 1000× Modified Trace Metal Solution 1 ml. All of the components were added together and dissolved in diH₂O. This solution was autoclaved. The pH was adjusted to 7.0 with ammonium hydroxide (30%) and q.s. to volume. Glucose 10 g, thiamine*HCl 0.1 g, and antibiotics were added after sterilization and pH adjustment.

### 1000× Modified Trace Metal Solution:

1000× Modified Trace Metal Solution contained citric Acids*H₂O 40 g, MnSO₄*H₂O 30 g, NaCl 10 g, Fe50₄*7H₂O 1 g, CoCl₂*6H₂O 1 g, ZnSO₄*7H₂O 1 g, CuSO₄*5H₂O 100 mg, H₃BO₃ 100 mg, and NaMoO₄*2H₂O 100 mg. Each component was dissolved one at a time in DI H₂O, pH to 3.0 with HCl/NaOH, then q.s. to volume and filter sterilized with a 0.22 micron filter.

Fermentation was performed in a 15-L bioreactor with BL21 (DE3) E. coli cells containing the upper mevalonic acid (MVA) pathway (pCL PtrcUpperPathway encoding E. faecalis mvaE and mvaS), the integrated lower MVA pathway (gi1.2KKDyI encoding S. cerevisiae mevalonate kinase, mevalonate phosphate kinase, mevalonate pyrophosphate decarboxylase, and IPP isomerase), and high expression of mevalonate kinase from M. mazei and isoprene synthase from Kudzu (pTrcKudzuMVK(M. mazei)). This experiment was carried out to monitor isoprene formation from glucose at the desired fermentation pH 7.0 and temperature 30° C. An inoculum of E. coli strain taken from a frozen vial was streaked onto an LB broth agar plate (with antibiotics) and incubated at 37° C. A single colony was inoculated into tryptone-yeast extract medium. After the inoculum grew to OD 1.0, measured at 550 nm, 500 mL was used to innoculate 5-L of cell medium in the 15-L bioreactor. In particular, the 15-L bioreactor had an initial working volume of 5 L. The liquid volume increases throughout the fermentation (such as to approximately 10 liters).

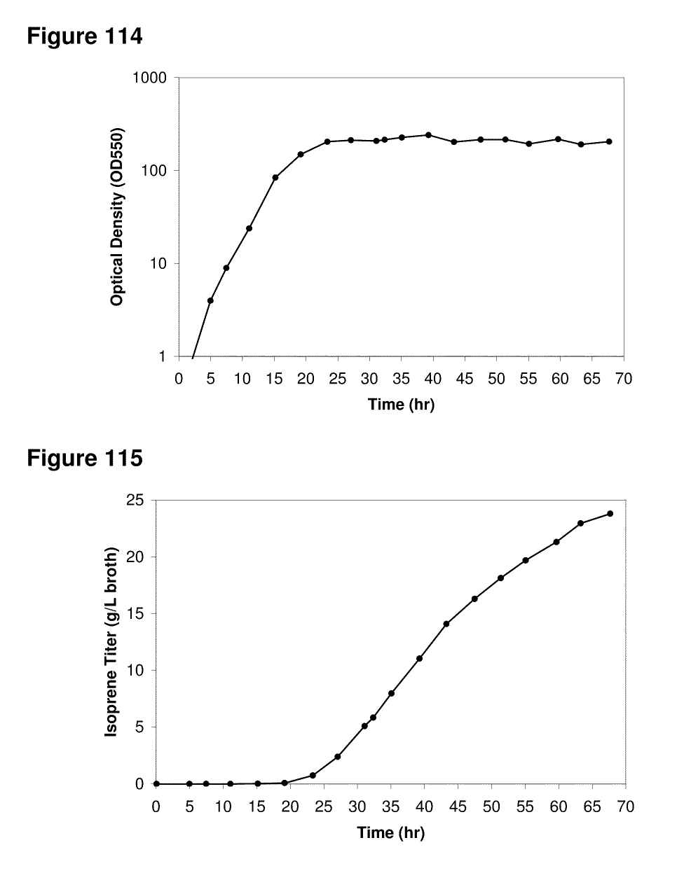

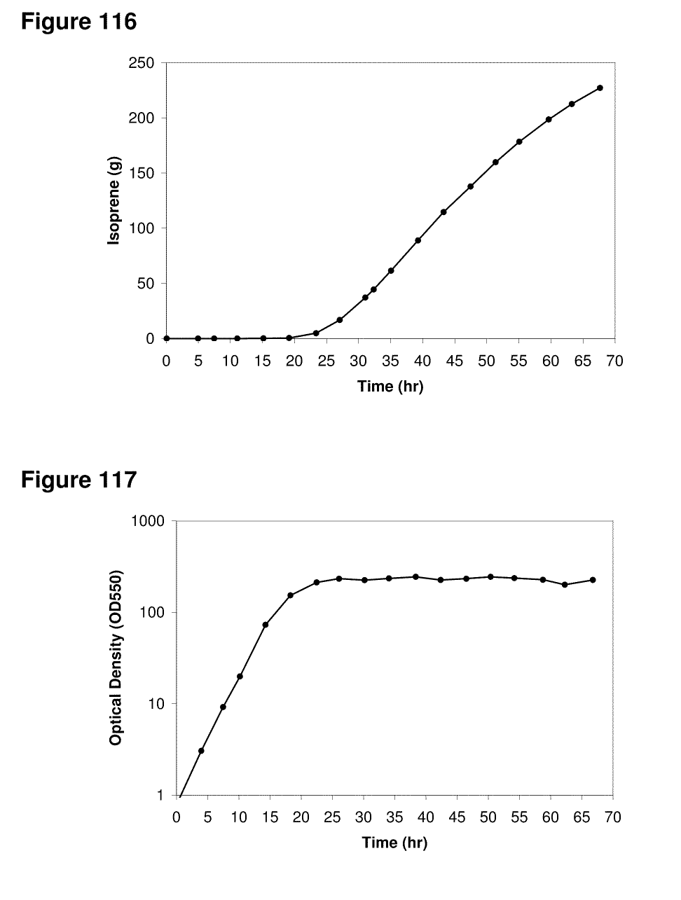

Glucose was fed at an exponential rate until cells reached the stationary phase. After this time the glucose feed was decreased to meet metabolic demands. The total amount of glucose delivered to the bioreactor during the 68 hour fermentation was 3.8 kg. Induction was achieved by adding isopropyl-beta-D-1-thiogalactopyranoside (IPTG). The IPTG concentration was brought to 51 uM when the optical density at 550 nm (OD₅₅₀) reached a value of 9. The IPTG concentration was raised to 88 uM when OD₅₅₀ reached 149. Additional IPTG additions raised the concentration to 119 uM at OD₅₅₀=195 and 152 uM at OD₅₅₀=210. The OD₅₅₀ profile within the bioreactor over time is shown in FIG. 114. The isoprene level in the off gas from the bioreactor was determined using a Hiden mass spectrometer. The isoprene titer increased over the course of the fermentation to a final value of 23.8 g/L (FIG. 115). The total amount of isoprene produced during the 68 hour fermentation was 227.2 g and the time course of production is shown in FIG. 116. The molar yield of utilized carbon that went into producing isoprene during fermentation was 13.0%. The weight percent yield of isoprene from glucose was 6.3%.

### Example 4

### Production of Isoprene by E. coli Expressing the Upper Mevalonic Acid (MVA) Pathway, the Integrated Lower MVA Pathway (gi1.2KKDyI), Mevalonate Kinase from M. mazei, and Isoprene Synthase from Kudzu and Grown in Fed-Batch Culture at the 15-L Scale

### Medium Recipe (Per Liter Fermentation Medium):

Each liter of fermentation medium contained K₂HPO₄ 7.5 g, MgSO₄*7H₂O 2 g, citric acid monohydrate 2 g, ferric ammonium citrate 0.3 g, yeast extract 0.5 g, and 1000× Modified Trace Metal Solution 1 ml. All of the components were added together and dissolved in diH₂O. This solution was autoclaved. The pH was adjusted to 7.0 with ammonium hydroxide (30%) and q.s. to volume. Glucose 10 g, thiamine*HCl 0.1 g, and antibiotics were added after sterilization and pH adjustment.

1000× Modified Trace Metal Solution:

1000× Modified Trace Metal Solution contained citric Acids*H₂O 40 g, MnSO₄*H₂O 30 g, NaCl 10 g, FeSO₄*7H₂O 1 g, CoCl₂*6H₂O 1 g, ZnSO₄*7H₂O 1 g, CuSO₄*5H₂O 100 mg, H₃BO₃ 100 mg, and NaMoO₄*2H₂O 100 mg. Each component was dissolved one at a time in DI H₂O, pH to 3.0 with HCl/NaOH, then q.s. to volume and filter sterilized with a 0.22 micron filter.

Fermentation was performed in a 15-L bioreactor with BL21 (DE3) E. coli cells containing the upper mevalonic acid (MVA) pathway (pCL PtrcUpperPathway encoding E. faecalis mvaE and mvaS), the integrated lower MVA pathway (gi1.2KKDyI encoding S. cerevisiae mevalonate kinase, mevalonate phosphate kinase, mevalonate pyrophosphate decarboxylase, and IPP isomerase), and high expression of mevalonate kinase from M. mazei and isoprene synthase from Kudzu (pTrcKudzuMVK(M. mazei)). This experiment was carried out to monitor isoprene formation from glucose at the desired fermentation pH 7.0 and temperature 30° C. An inoculum of E. coli strain taken from a frozen vial was streaked onto an LB broth agar plate (with antibiotics) and incubated at 37° C. A single colony was inoculated into tryptone-yeast extract medium. After the inoculum grew to OD 1.0, measured at 550 nm, 500 mL was used to innoculate 5-L of cell medium in the 15-L bioreactor. The liquid volume increases throughout the fermentation (such as to approximately 10 liters).

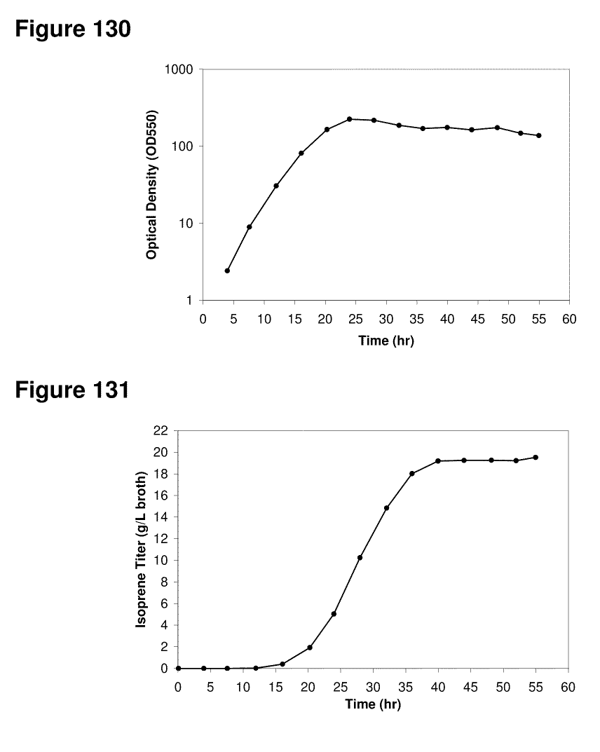

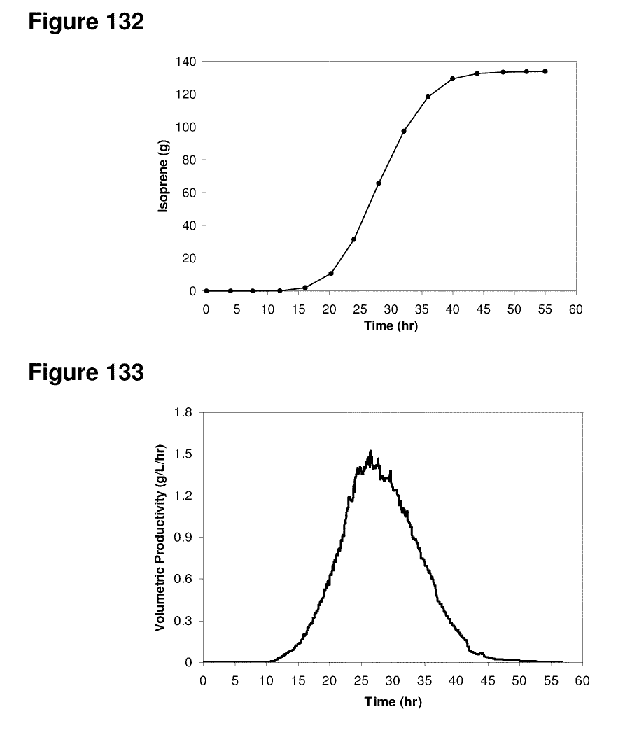

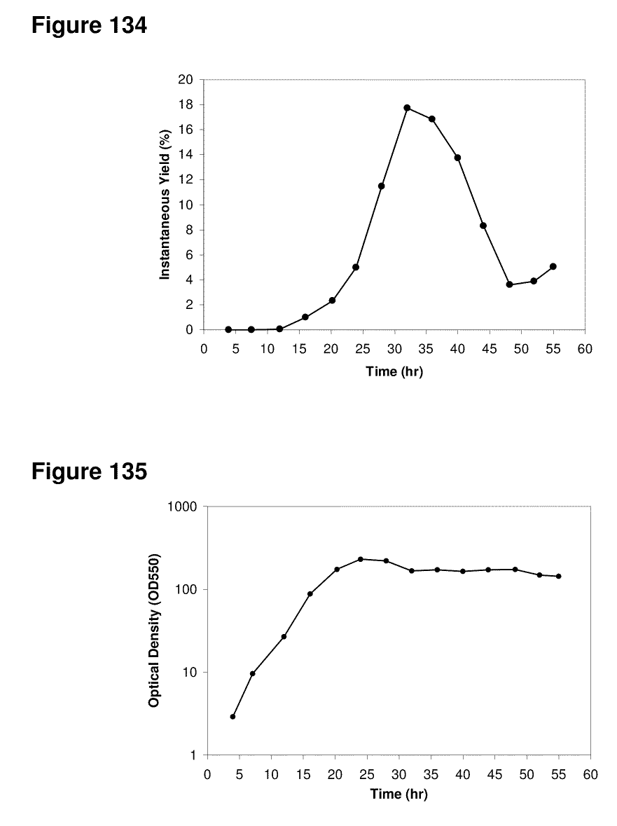

Glucose was fed at an exponential rate until cells reached the stationary phase. After this time the glucose feed was decreased to meet metabolic demands. The total amount of glucose delivered to the bioreactor during the 55 hour fermentation was 1.9 kg. Induction was achieved by adding IPTG. The IPTG concentration was brought to 111 uM when the optical density at 550 nm (OD₅₅₀) reached a value of 9. The IPTG concentration was raised to 193 uM when OD₅₅₀ reached 155. The OD₅₅₀ profile within the bioreactor over time is shown in FIG. 130. The isoprene level in the off gas from the bioreactor was determined using a Hiden mass spectrometer. The isoprene titer increased over the course of the fermentation to a final value of 19.5 g/L (FIG. 131). The total amount of isoprene produced during the 55 hour fermentation was 133.8 g, and the time course of production is shown in FIG. 132. Instantaneous volumetric productivity levels reached values as high as 1.5 g isoprene/L broth/hr (FIG. 133). Instantaneous yield levels reached as high as 17.7% w/w (FIG. 134). The molar yield of utilized carbon that went into producing isoprene during fermentation was 15.8%. The weight percent yield of isoprene from glucose over the entire fermentation was 7.4%.

### Example 5

### Production of Isoprene by E. coli Expressing the Upper Mevalonic Acid (MVA) Pathway, the Integrated Lower MVA Pathway (gi1.2KKDyI), Mevalonate Kinase from M. mazei, and Isoprene Synthase from Kudzu and Grown in Fed-Batch Culture at the 15-L Scale

### Medium Recipe (Per Liter Fermentation Medium):

Each liter of fermentation medium contained K₂HPO₄ 7.5 g, MgSO₄*7H₂O 2 g, citric acid monohydrate 2 g, ferric ammonium citrate 0.3 g, yeast extract 0.5 g, and 1000× Modified Trace Metal Solution 1 ml. All of the components were added together and dissolved in diH₂O. This solution was autoclaved. The pH was adjusted to 7.0 with ammonium hydroxide (30%) and q.s. to volume. Glucose 10 g, thiamine*HCl0.1 g, and antibiotics were added after sterilization and pH adjustment.

### 1000× Modified Trace Metal Solution:

1000× Modified Trace Metal Solution contained citric Acids*H₂O 40 g, MnSO₄*H₂O 30 g, NaCl 10 g, FeSO₄*7H₂O 1 g, CoCl₂*6H₂O 1 g, ZnSO₄*7H₂O 1 g, CuSO₄*5H₂O 100 mg, H₃BO₃ 100 mg, and NaMoO₄*2H₂O 100 mg. Each component was dissolved one at a time in DI H₂O, pH to 3.0 with HCl/NaOH, then q.s. to volume and filter sterilized with a 0.22 micron filter.

Fermentation was performed in a 15-L bioreactor with BL21 (DE3) E. coli cells containing the upper mevalonic acid (MVA) pathway (pCL PtrcUpperPathway encoding E. faecalis mvaE and mvaS), the integrated lower MVA pathway (gi1.2KKDyI encoding S. cerevisiae mevalonate kinase, mevalonate phosphate kinase, mevalonate pyrophosphate decarboxylase, and IPP isomerase), and high expression of mevalonate kinase from M. mazei and isoprene synthase from Kudzu (pTrcKudzuMVK(M. mazei)). This experiment was carried out to monitor isoprene formation from glucose at the desired fermentation pH 7.0 and temperature 30° C. An inoculum of E. coli strain taken from a frozen vial was streaked onto an LB broth agar plate (with antibiotics) and incubated at 37° C. A single colony was inoculated into tryptone-yeast extract medium. After the inoculum grew to OD 1.0, measured at 550 nm, 500 mL was used to innoculate 5-L of cell medium in the 15-L bioreactor. The liquid volume increases throughout the fermentation (such as to approximately 10 liters).

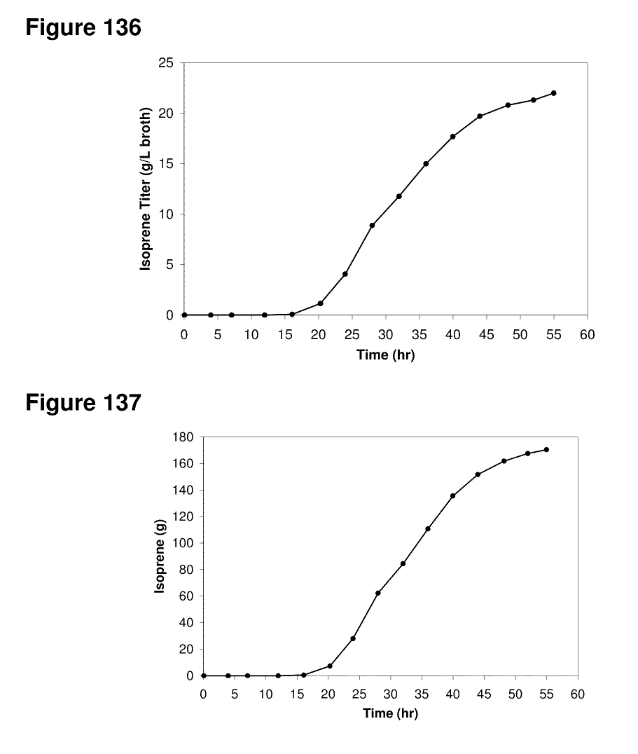

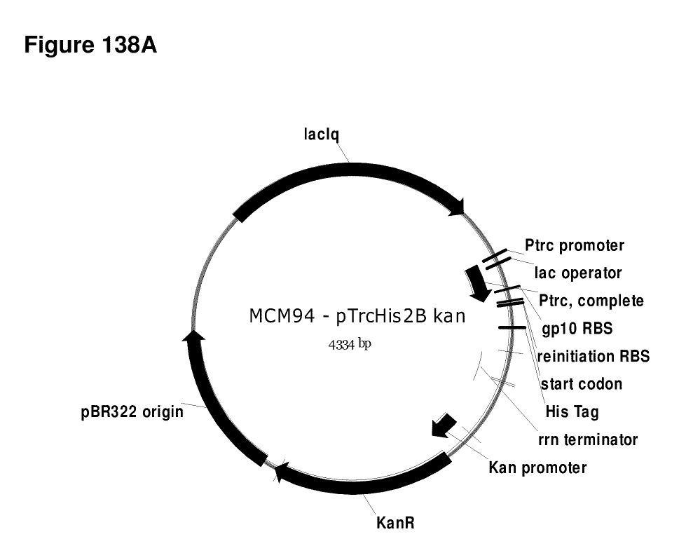

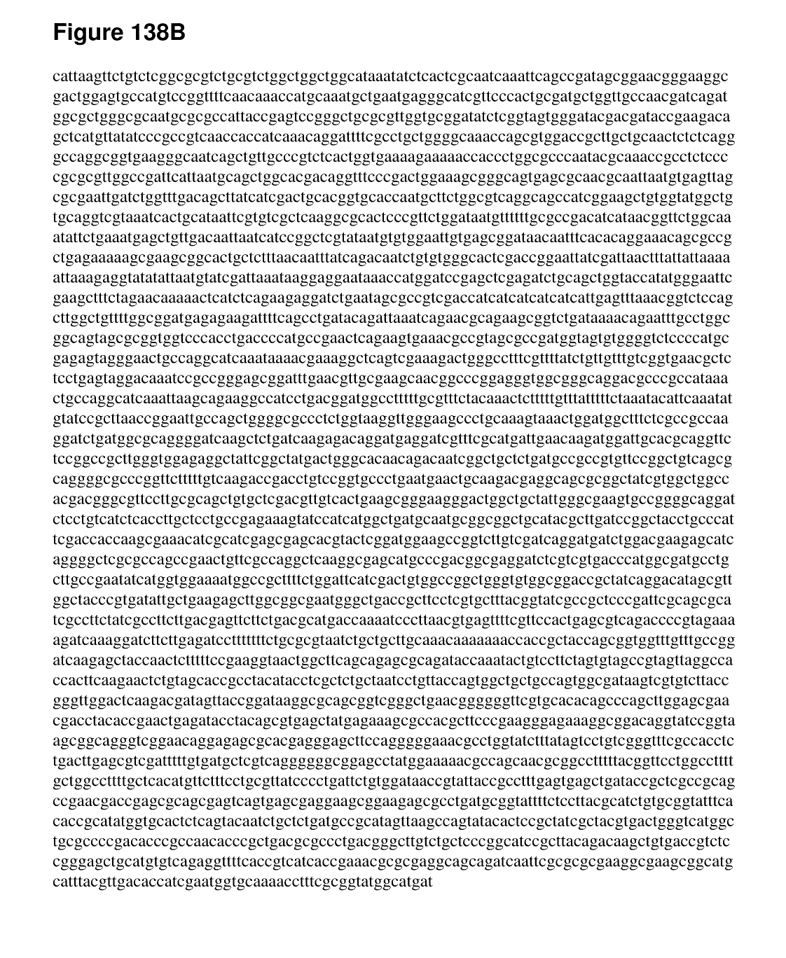

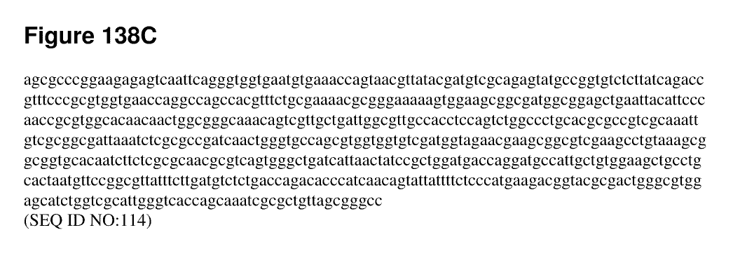

Glucose was fed at an exponential rate until cells reached the stationary phase. After this time the glucose feed was decreased to meet metabolic demands. The total amount of glucose delivered to the bioreactor during the 55 hour fermentation was 2.2 kg. Induction was achieved by adding IPTG. The IPTG concentration was brought to 51 uM when the optical density at 550 nm (OD₅₅₀) reached a value of 10. In addition to the IPTG spike, at OD₅₅₀=10 a constant feed began and delivered 164 mg of IPTG over 18 hours. The OD₅₅₀ profile within the bioreactor over time is shown in FIG. 135. The isoprene level in the off gas from the bioreactor was determined using a Hiden mass spectrometer. The isoprene titer increased over the course of the fermentation to a final value of 22.0 g/L (FIG. 136). The total amount of isoprene produced during the 55 hour fermentation was 170.5 g and the time course of production is shown in FIG. 137. The molar yield of utilized carbon that went into producing isoprene during fermentation was 16.6%. The weight percent yield of isoprene from glucose over the entire fermentation was 7.7%.

### Example 6

### Over-Expression of Mevalonate Kinase and Isoprene Synthase in E. coli Harboring the MVA Pathway

Over-expression of both mevalonate kinase and isoprene synthase results in high specific productivity of isoprene production by E. coli harboring the MVA pathway

### I. Construction of Plasmid MCM94

Plasmid pTrcHis2B (Invitrogen) was digested for 2 hours at 30° C. in 10 uL containing ApaI (Roche) and Roche BufferA. The reaction was brought to a total of 30 uL containing 1× Roche Buffer H and 2 uL PstI (Roche) and incubated for 1 hour at 37° C. The 996 by fragment containing the pTrc promoter region was gel purified from an Invitrogen E-gel (1.2%) using a Qiagen Gel Purification spin column according to the manufacturer's protocol.

Plasmid MCM29 was digested as described above, and the 3338 bp fragment containing the origin and kanR genes was gel purified as described above. The two fragments (3 uL pTrcHis2B fragment, 1 uL MCM29 fragment) were ligated for 1 hour at room temperature in a 20 uL reaction following the Roche Rapid DNA Ligation kit protocol. 5 uL of this ligation reaction was used to transform Invitrogen TOP10 chemically competent cells according to the manufacturer's protocol. Transformants were selected on LA and kanamycin50 ppm. Plasmids were isolated by Qiagen Spin Miniprep from several colonies which had been grown overnight in 5 mL LB and kan50. A clone with the pTrc promoter but no kudzu isoprene synthase gene was frozen as MCM94.

### II. Construction of Strains MCM433, 437, and 438

Plasmid pCL PtrcUpperHGS2 (Construction of this plasmid is described in Example 1, part VI) was transformed into MCM331 by electroporation as described herein for expression strain MCM401. Transformant MCM433 was selected on LA and spectinomycin 50 ppm. Strain MCM433 was subsequently transformed with either plasmid MCM94 (described above) or MCM376 and selected on LA, spectinomycin 50 ppm, and kanamycin 50 ppm.

TABLE 8
Strains MCM433, 437, and 438

| Strain | Parent | Host Origin | Integrated | Plasmid(s) | Markers |
|---|---|---|---|---|---|
| MCM433 | MCM331 | BL21(DE3) | gi1.2KKDyI | pCLUpperHGS2 | cmp5, |
| | | | | | spec50 |
| MCM437 | MCM433 | BL21(DE3) | gi1.2KKDyI | pCLUpperHGS2 | cmp5, |
| | | | | pTrcHis2B kan | spec50. |
| | | | | (MCM94) | kan50 |
| MCM438 | MCM433 | BL21(DE3) | gi1.2KKDyI | pCLUpperHGS2 | cmp5, |
| | | | | pTrcKudzuMVK(mazei) | spec50. |
| | | | | MCM376 | kan50 |

### III. Cell Fermentation

### Medium Recipe (Per Liter Fermentation Medium):

Each liter of fermentation medium contained K₂HPO₄ 13.6 g, KH₂PO₄ 13.6 g, MgSO₄*7H₂O 2 g, citric acid monohydrate 2 g, ferric ammonium citrate 0.3 g, (NH₄)₂SO₄ 3.2 g, yeast extract 1 g, and 1000× Trace Metal Solution 1 ml. All of the components were added together and dissolved in diH₂O. The pH was adjusted to 6.8 with ammonium hydroxide (30%) and brought to volume. Media was filter sterilized with a 0.22 micron filter. Glucose 5.0 g and antibiotics were added after sterilization and pH adjustment.

### 1000× Trace Metal Solution (Per Liter Fermentation Media):

1000× Trace Metal Solution contained citric Acids*H₂O 40 g, MnSO₄*H₂O 30 g, NaCl 10 g, FeSO₄*7H₂O 1 g, CoCl₂*6H₂O 1 g, ZnSO₄*7H₂O 1 g, CuSO₄*5H₂O 100 mg, H₃BO₃ 100 mg, and NaMoO₄*2H₂O 100 mg. Each component was dissolved one at a time in DI H₂O, pH to 3.0 with HCl/NaOH, then brought to volume and filter sterilized with a 0.22 micron filter.

### Strains:

The MCM343 strain is BL21 (DE3) E. coli cells containing the upper mevalonic acid (MVA) pathway (pCL PtrcUpperPathway encoding E. faecalis mvaE and mvaS), the integrated lower MVA pathway (gi1.2KKDyI encoding S. cerevisiae mevalonate kinase, mevalonate phosphate kinase, mevalonate pyrophosphate decarboxylase, and IPP isomerase), and isoprene synthase from Kudzu (pTrcKudzu). This strain has low MVK polypeptide activity and high isoprene synthase polypeptide activity.

The MCM401 strain is BL21 (DE3) E. coli cells containing the upper MVA pathway (pCL PtrcUpperPathway), the integrated lower MVA pathway (gi1.2KKDyI), and high expression of MVK from M. mazei and IS from Kudzu (pTrcKudzuMVK(M. mazei). This strain has high MVK polypeptide activity and high isoprene synthase polypeptide activity.

The MCM437 strain is BL21 (DE3) E. coli cells containing the upper MVA pathway and low expression of IS from Kudzu (pCLPtrcUpperPathwayHGS2), the integrated lower MVA pathway (gi1.2KKDyI), and a control plasmid conferring kanamycin resistance (so that the growth media was identical in all cases). This strain has low MVK polypeptide activity and low isoprene synthase.

The MCM438 strain is BL21 (DE3) E. coli cells containing the upper MVA pathway and low expression of IS from Kudzu (pCLPtrcUpperPathwayHGS2), the integrated lower MVA pathway (gi1.2KKDyI), and strong expression of M. mazei MVK (M. mazei MVK in pET200). This strain has high MVK polypeptide activity and low isoprene synthase polypeptide activity.

Isoprene production was analyzed by growing the strains in a Cellerator™ from MicroReactor Technologies, Inc. The working volume in each of the 24 wells was 4.5 mL. The temperature was maintained at 30° C., the pH setpoint was 7.0, the oxygen flow setpoint was 20 sccm and the agitation rate was 800 rpm. An inoculum of E. coli strain taken from a frozen vial was streaked onto an LB broth agar plate (with antibiotics) and incubated at 30° C. A single colony was inoculated into media with antibiotics and grown overnight. The bacteria were diluted into 4.5 mL of media with antibiotics to reach an optical density of 0.05 measured at 550 nm.

Off-gas analysis of isoprene was performed using a gas chromatograph-mass spectrometer (GC-MS) (Agilent) headspace assay. Sample preparation was as follows: 100 μL of whole broth was placed in a sealed GC vial and incubated at 30° C. for a fixed time of 30 minutes. Following a heat kill step, consisting of incubation at 70° C. for 5 minutes, the sample was loaded on the GC.

Optical density (OD) at a wavelength of 550 nm was obtained using a microplate reader (Spectramax) during the course of the run. Specific productivity was obtained by dividing the isoprene concentration (μg/L) by the OD reading. Samples were taken at three time points for each of the 24-wells over the course of the mini-fermentations. There were six replicates for each strain (4 strains×6 wells/strain).

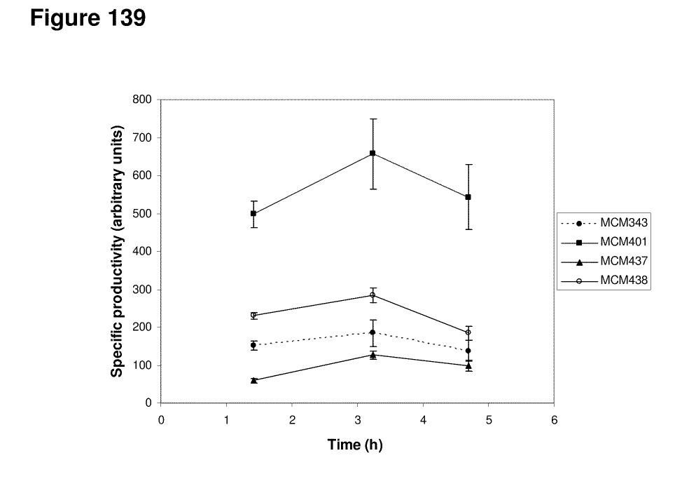

Specific productivity of isoprene from a strain expressing the full mevalonic acid pathway and Kudzu isoprene synthase at low levels (MCM437) was compared to a strain that in addition over-expressed MVK from M. mazei and Kudzu isoprene synthase (MCM401), as well as strains that either over-expressed just MVK (MCM438), or just Kudzu isoprene synthase (MCM343). The bacteria were grown under identical conditions in defined media with glucose as a carbon source in mini-fermentations. Induction of isoprene production was achieved by adding IPTG to a final concentration of 200 μM at the start of the run. Headspace measurements over time (FIG. 139) revealed that the strain over-expressing both MVK and isoprene synthase (MCM401) had higher specific productivity of isoprene compared to the strain over-expressing just MVK (MCM438) or just Kudzu isoprene synthase (MCM343). The strain with low activities of both MVK and Kudzu isoprene synthase (MCM437) had the lowest specific productivity of isoprene overall.

### IV. Determination of Isoprene Synthase Activity and Volumetric Productivity in Fermentation Runs.

Strain MCM401 that overexpresses both M. mazei MVK and isoprene synthase had a greater maximum volumetric productivity for isoprene than either strain MC343 or strain MCM127 that do not express M. mazei MVK.

Isoprene Synthase DMAPP Activity from Lysate Protocol

For this assay, the following reagents were used: 50% glycerol in PEB containing 1 mg/mL lysozyme (Sigma) and 0.1 mg/mL DNAaseI (Sigma). 1 mL of fermentation broth was mixed with 1 mL of 50% glycerol in PEB containing 1 mg lysozyme and 0.1 mg DNAaseI. The mixture is passed through the french press one time. 25 μL of the mixture is then used for the DMAPP assay. The DMAPP assay contained the following components:

### DMAPP Assay

25 μL lysate mixture
5 μL MgCl₂ (1 M)
5 μL DMAPP (100mM)
65 μL 50 mM Tris pH 8
Total volume: 100 μL

The reaction is performed at 30° C. for 15 minutes in a gas tight 1.8 mL GC tube. Reactions are terminated by the addition of 100 μL 250 mM EDTA (pH 8).

The active protein concentration was measured using Equation 14.

mg/mL active isoprene synthase=(Dilution factor)*X ug/L (DMAPP Assay reading)*0.0705/294(specific activity from 14-L) or 0.0002397*X ug/L Equation 14

The volumetric productivity was measured using Equation 15.

mg/L/h isoprene=(dilution factor)*0.288*X ug/L (DMAPP Assay reading) Equation 15

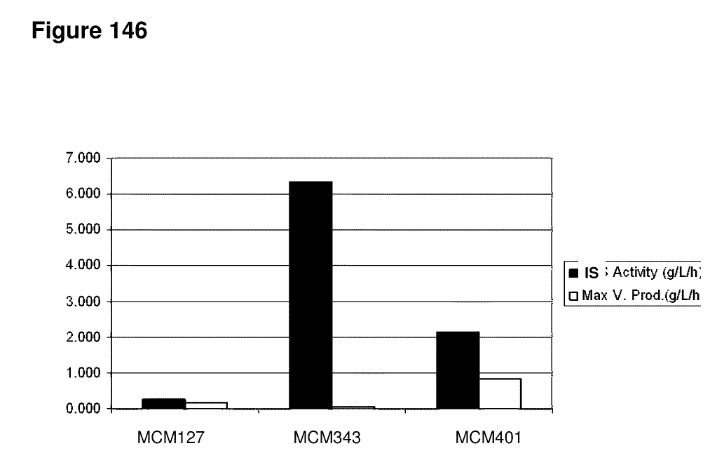

The maximum in vitro isoprene synthase polypeptide activity was compared with the maximum volumetric productivity for strains MCM401, MC343, and MCM127 (FIG. 146).

### Example 7

### Exemplary Methods for Producing Isoprene: Isoprene Fermentation from E. coli Expressing Genes from the Mevalonic Acid Pathway and Grown in Fed-Batch Culture at the 15-L Scale

### Medium Recipe (Per Liter Fermentation Medium):

Each liter of fermentation medium contained K₂HPO₄ 7.5 g, MgSO₄*7H₂O 2 g, citric acid monohydrate 2 g, ferric ammonium citrate 0.3 g, yeast extract 0.5 g, 1000× Modified Trace Metal Solution 1 ml. All of the components were added together and dissolved in diH2O. This solution was autoclaved. The pH was adjusted to 7.0 with ammonium hydroxide (30%) and brought to volume. Glucose 10 g, thiamine*HCl 0.1 g, and antibiotics were added after sterilization and pH adjustment.

### 1000× Modified Trace Metal Solution:

1000× Trace Metal Solution contained citric Acids*H₂O 40 g, MnSO₄*H₂O 30 g, NaCl 10 g, FeSO₄*7H₂O 1 g, CoCl₂*6H₂O 1 g, ZnSO*7H₂O 1 g, CuSO4*5H₂O 100 mg, H₃BO₃ 100 mg, NaMoO₄*2H₂O 100 mg. Each component was dissolved one at a time in Di H2O, pH to 3.0 with HCl/NaOH, then brought to volume and filter sterilized with 0.22 micron filter.

I. MCM343 High Titer: Isoprene Fermentation from E. coli Expressing Genes from the Mevalonic Acid Pathway and Grown in Fed-Batch Culture at the 15-L Scale

Fermentation was performed in a 15-L bioreactor with BL21 (DE3) E. coli cells containing the gi1.2 integrated lower MVA pathway and the pCL PtrcUpperMVA and pTrcKudzu plasmids. This experiment was carried out to monitor isoprene formation from glucose at the desired fermentation pH 7.0 and temperature 30° C. An inoculum of E. coli strain taken from a frozen vial was streaked onto an LB broth agar plate (with antibiotics) and incubated at 37° C. A single colony was inoculated into tryptone-yeast extract medium. After the inoculum grew to OD 1.0, measured at 550 nm, 500 mL was used to inoculate a 5-L bioreactor.

Glucose was fed at an exponential rate until cells reached the stationary phase. After this time the glucose feed was decreased to meet metabolic demands. The total amount of glucose delivered to the bioreactor during the 58 hour fermentation was 4.5 kg. Induction was achieved by adding isopropyl-beta-D-1-thiogalactopyranoside (IPTG). The IPTG concentration was brought to 98 uM when the carbon dioxide evolution rate reached 25 mmol/L/hr (OD₅₅₀=9). The OD₅₅₀ profile within the bioreactor over time is shown in FIG. 112C. The isoprene level in the off gas from the bioreactor was determined using a Hiden mass spectrometer. The isoprene titer increased over the course of the fermentation to a final value of 1.6 g/L (FIG. 112D). The total amount of isoprene produced during the 58 hour fermentation was 17.9 g and the time course of production is shown in FIG. 112E. The molar yield of utilized carbon that went into producing isoprene during fermentation was 0.8%. The weight percent yield of isoprene from glucose was 0.4%.

II. MCM127: Isoprene Fermentation from E. coli Expressing Genes from the Mevalonic Acid Pathway and Grown in Fed-Batch Culture at the 15-L Scale

Fermentation was performed in a 15-L bioreactor with BL21 (DE3) E. coli cells containing the pCL PtrcUpperMVA and pTrc KKDyIkIS plasmids. This experiment was carried out to monitor isoprene formation from glucose at the desired fermentation pH 7.0 and temperature 30° C. An inoculum of E. coli strain taken from a frozen vial was streaked onto an LB broth agar plate (with antibiotics) and incubated at 37° C. A single colony was inoculated into tryptone-yeast extract medium. After the inoculum grew to OD 1.0, measured at 550 nm, 500 mL was used to inoculate a 5-L bioreactor.

Glucose was fed at an exponential rate until cells reached the stationary phase. After this time the glucose feed was decreased to meet metabolic demands. The total amount of glucose delivered to the bioreactor during the 43 hour fermentation was 1.4 kg. Induction was achieved by adding isopropyl-beta-D-1-thiogalactopyranoside (IPTG). The IPTG concentration was brought to 23 uM when the carbon dioxide evolution rate reached 25 mmol/L/hr (OD₅₅₀=129). The OD₅₅₀ profile within the bioreactor over time is shown in FIG. 112F. The isoprene level in the off gas from the bioreactor was determined as previously described by measuring isoprene concentrations in the offgas by GC. The isoprene titer increased over the course of the fermentation to a final value of 0.4 g/L (FIG. 112G). The total amount of isoprene produced during the 43 hour fermentation was 3.0 g and the time course of production is shown in FIG. 112H. The molar yield of utilized carbon that went into producing isoprene during fermentation was 0.5%. The weight percent yield of isoprene from glucose was 0.3%.

III. dxr Knock-Out Strain: Isoprene Fermentation from E. coli Expressing Genes from the Mevalonic Acid Pathway and Grown in Fed-Batch Culture at the 15-L Scale.

Fermentation was performed in a 15-L bioreactor with BL21 (DE3) E. coli cells (Δdxr) containing the pCL PtrcUpperMVA and pTrc KKDyIkIS plasmids. This experiment was carried out to monitor isoprene formation from glucose at the desired fermentation pH 7.0 and temperature 30° C. An inoculum of E. coli strain taken from a frozen vial was streaked onto an LB broth agar plate (with antibiotics) and incubated at 37° C. A single colony was inoculated into tryptone-yeast extract medium. After the inoculum grew to OD 1.0, measured at 550 nm, 500 mL was used to inoculate a 15-L bioreactor containing an initial volume of 5-L

Glucose was fed at an exponential rate until cells reached the stationary phase. After this time the glucose feed was decreased to meet metabolic demands. The total amount of glucose delivered to the bioreactor during the 43 hour fermentation was 1.7 kg. Induction was achieved by adding isopropyl-beta-D-1-thiogalactopyranoside (IPTG). The IPTG concentration was brought to 25 uM when the optical density at 550 nm (OD₅₅₀) reached a value of 8. The IPTG concentration was raised to 40 uM when OD₅₅₀ reached 140. The OD₅₅₀ profile within the bioreactor over time is shown in FIG. 112I. The isoprene level in the off gas from the bioreactor was determined as previously described (GC of offgas samples). The isoprene titer increased over the course of the fermentation to a final value of 0.9 g/L (FIG. 112J). The total amount of isoprene produced during the 43 hour fermentation was 6.0 g and the time course of production is shown in FIG. 112K. The molar yield of utilized carbon that went into producing isoprene during fermentation was 0.8%. The weight percent yield of isoprene from glucose was 0.4%.

(i) Construction of the dxr Mutant in E. coli

To generate a deletion of dxr (1-deoxy-D-xylulose 5-phosphate reductoisomerase), the enzyme that encodes the first committed step in the deoxy-xylulose-phosphate (DXP) pathway in Escherichia coli, the GeneBridges Quick & Easy E. coli Gene Deletion Kit (GB) was used according to the manufacturer's recommended protocol. Briefly, GB insertion cassettes encoding either kanamycin (FRT-PGK-gb2-neo-FRT) or chloramphenicol (FRT-cm-FRT) resistance were PCR amplified using primers GBdxr1 and GBdxr2 (see below for primer sequences and cycling parameters). PCR products of the correct size (for the respective GB insertion cassette) were pooled, purified (Qiagen) and diluted to a concentration of approximately 300 ng/μl. The deletion of dxr was then carried out according to the protocol described in the GB manual. All replicating plasmids were introduced into E. coli strains via electroporation using standard molecular biology techniques (see Table 16 below for a complete strain list). LB medium containing ampicillin (50 μg/ml) and spectinomycin (50 μg/ml) was inoculated with E. coli strains (DW13 or DW38) harboring the pRed/ET plasmid (encoding ampicillin/carbenicillin resistance) and pCL Ptrc(minus lacO) KKDyI (from Edwin Lee, encoding spectinomycin resistance). These strains carried pCL Ptrc(minus lacO) KKDyI (see (iv) below) so that E. coli, in the absence of a functional DXP pathway, could convert mevalonic acid (MVA) through the MVA lower pathway to IPP/DMAPP as a source for all lower isoprenoid molecules. Cultures were grown overnight at 30° C. and diluted to an OD₆₀₀ of approximately 0.2 in 5 ml total volume with antibiotics the next morning. After several hours of growth at 30° C., strains were shifted to 37° C. and L-arabinose was added at a concentration of 0.4%. After 1 hour of induction, cells were washed multiple times in ice cold H₂O, and approximately 700 ng of the purified PCR product (described above) for each GB insertion template was introduced via electroporation (using standard techniques). Cells were recovered for 3 hours at 37° C. in LB with 1 mM MVA with no antibiotics, and then plated onto selective LB medium (MVA 1 mM and spectinomycin 50 μg/ml, with either kanamycin 15 μg/ml or chloramphenicol 25 μg/ml). The next day, positive colonies were tested by PCR, using the dxrTest1 and dxrTest2 primers, with either GBprimer2 or GBprimerDW (i.e. GB3, see FIG. 112M), respectively (see Table 16). Colonies that tested positive with these primer combinations were then tested for sensitivity to MVA at varying concentrations. FIG. 112J shows that in the absence of MVA, dxr deletion strains are unable to grow, whereas in the presence of 1 mM MVA, growth is robust. FIG. 112N also shows that at a concentration of 10 mM MVA, growth of dxr deletion strains appears to be inhibited, most likely because of the accumulation of isoprenoid molecules. To generate strain DW48, strain DW43 was electroporated with plasmids MCM82 (Sp) and MCM118 (Kan), which harbor the entire MVA pathway and HGS. Since MVA was omitted from recovery and on the selective plate (LB with Sp μg/ml and Kan μg/ml), strain DW48 was forced to lose plasmid pCL Ptrc(minus lacO) KKDyI and gain MCM82, which contains the MVA upper pathway. Thus, only cells harboring the entire MVA pathway to convert acetyl-CoA to IPP/DMAPP and lower isoprenoids were able to grow without exogenous MVA.

### (ii) PCR Cycling Parameters

The Herculase II (Stratagene) DNA polymerase enzyme was used for amplification of all GB templates with oligonucleotide primer pairs at a concentration of 0.4 μM each in 50 μl total volume/reaction according to the manufacturer's protocol. All PCR products for generating dxr deletion strains via GB were of the expected size: approximately 1.6 kb (kanamycin), and 1.5 kb (chloramphenicol).

To test GB insertions at the dxr locus, illustra PuReTaq Ready-To-Go™ PCR Beads (GE Healthcare) were used with oligonucleotide primer pairs at a concentration of 0.4 μM each in 25 μl total volume/reaction.
1) 95° C.-4 min
2) 95° C.-20 sec
3) 55° C.-20 sec (52° C. for Beads)
4) 72° C.-2 min (30 sec for Beads)
5 cycles of steps 2 through 4
5) 95° C.-20 sec
6) 58° C.-20 sec (55° C. for Beads)
7) 72° C.-2 min (30 sec for Beads)
25 cycles of steps 5 through 7
72° C.-10 min
4° C.-end

TABLE 16
PCR primers, plasmids, and Strains

| Primer Name | Sequence (5′ to 3′) | Purpose |
|---|---|---|
| GBdxr1 | GGCTGGCGGCGTTTTGCTTTTTATT | dxr knock out GB - Forward primer for |
| | CTGTCTCAACTCTGGATGTTTCATG | all templates |
| | AATTAACCCTCACTAAAGGGCG | |
| | (SEQ ID NO: 146) | |
| GBdxr2 | AAGCCCTACGCTAACAAATAGCGC | dxr knock out GB - Reverse primer for |
| | GACTCTCTGTAGCCGGATTATCCTC | all GB templates |
| | ATAATACGACTCACTATAGGGCTC | |
| | (SEQ ID NO: 147) | |
| dxrTest1 | ACGCCGCTCAGTAGATCCTTGCGG | 5′ of 50 bp homology region (in GBdxr1) |
| | AT | used for GB knock-out |
| | (SEQ ID NO: 148) | |
| dxrTest2 | CTACTTACGATCAGATGGCGCAGA | 3′ of 50 bp homology region (in GBdxr2) |
| | CTA | used for GB knock-out |
| | (SEQ ID NO: 149) | |
| GBprimer2 | CGAGACTAGTGAGACGTGCTAC | GB test primer all cassettes - amplifies |
| | (SEQ ID NO: 150) | towards 5′ end |
| GBprimerDW | AAAGACCGACCAAGCGACGTCTGA | GB test primer all cassettes - amplifies |
| | (SEQ ID NO: 151) | towards 3′ end |
| Plasmid | Resistance | purpose |
| pCL Ptrc(minus | Spectinomycin (sp) | Lower MVA pathway for conversion of |
| lacO) KKDyI | | MVA to IPP/DMAPP - lower isoprenoids |
| FRT-cm-FRT | Chloramphenicol (GBchlor) | GB template - chloramphenicol |
| FRT-PGK-gb2- | Kanamycin (GBkan) | GB template - kanamycin |
| neo-FRT | | |
| pRedET | Ampicillin (amp) | GB L-arabinose inducible expression of |
| | | Red/ET proteins |
| MCM82 | Spectinomycin (sp) | Upper MVA pathway |
| MCM118 | Kanamycin (kan) | Lower MVA pathway + HGS |
| Strain | Genotype | purpose |
| DW13 | MG1655 with pCL Ptrc(minus lacO) | Parent strain of dxr deletion - has entire |
| | KKDyI and pRedET, sp, amp | MVA lower pathway |
| DW23 | MG1655 Δdxr::GBkan with pCL | dxr delection (kan) in MG1655 |
| | Ptrc(minus lacO) KKDyI, kan, sp | |
| DW28 | MG1655 Δdxr::GBchlor with pCL | dxr delection (chlor) in MG1655 |
| | Ptrc(minus lacO) KKDyI, chlor, sp | |
| DW38 | BL21 DE3 (Invitrogen) with pCL | Parent strain of dxr deletion - has entire |
| | Ptrc(minus lacO) KKDyI and pRedET, | MVA lower pathway |
| | sp, amp | |
| DW43 | BL21 DE3 Δdxr::GBchlor with pCL | dxr delection (chlor) in BL21 DE3 |
| | Ptrc(minus lacO) KKDyI, chlor, sp | |
| DW48 | BL21 DE3 Δdxr::GBchlor with MCM82 | dxr delection (chlor) in BL21 DE3 with |
| | and MCM118, sp, kan | entire MVA pathway - requires no MVA |

(iii) Construction of MCM184—pCL Ptrc(minus lacO) UpperPathway

Plasmid MCM82 was mutagenized using the Stratagene QuikChange XL II kit. A reaction consisting of 10 uL buffer, 1 uL 100 ng/uL MCM82 DNA, 2.5 uL 10 uM primer MCM63 (SEQ ID NO:139), 2.5 uL 10 uM primer MCM64 (SEQ ID NO:140), 2 uL dNTP mix, 6 uL QuikSolution, 76 uL ddH2O and 2 uL polymerase was combined and aliquotted to four PCR tubes. Tubes were cycled in columns 1, 4, 7 and 12 of a BioRad 96-well gradient block using 1× 95C for 1 minute, 18×95° C. for 50 seconds, 60-65° C. for 50 seconds, 68° C. for 10 minute, 1× 68° C. for 7 minutes, 1× 4° C. until cool. 1 uL DpnI was added and reactions were incubated at 37° C. for 2 hr and then frozen overnight at −20° C. 5 uL was transformed into Invitrogen TOP10 OneShot cells according to the manufacturer's protocol. Transformants were selected on LA+50 ppm Spectinomycin. Several colonies were cultured in LB+spectinomycin50 and then used for plasmid purification. Clone 2 from reaction 3 (column 7 from gradient block PCR) had the expected sequence and was frozen as MCM184.

(iv) Construction of pCL Ptrc(ΔlacO) KKDyI
(as referred to as pCL Ptrc (minus lacO) KKDyI or pCL Ptrc (minus lacO) Lower Pathway)

Plasmid MCM184 (pCL Ptrc(minus lacO) UpperPathway) was digested sequentially with SacI and PstI restriction endonucleases to remove the Upper MVA Pathway. A reaction consisting of 8 uL MCM184 (80 ng/uL), 3 ul Roche 10× Buffer A, 2 uL SacI restriction endonuclease, and 17 uL ddH₂O was prepared and incubated at 37° C. for 2 hours. The SacI restriction endonuclease was then inactivated by heating at 65° C. for 20 minutes. The DNA fragment was then purified by using a Qiagen PCR Purification column per manufacturer's protocol. The DNA fragment was then eluted from the column with a volume of 34 uL ddH₂O. The next (sequential) restriction digest reaction consisted of the 34 uL SacI digested eluant, 4 uL Roche 10× Buffer H, and 2 uL PstI restriction endonuclease. The reaction was incubated at 37° C. for 2 hours before being heat inactivated at 65° C. for 20 minutes. A dephosphorylation step was then performed by addition of 4.7 uL Roche 10× Shrimp Alkaline Phosphatase (SAP) buffer), and 2 uL SAP enzyme. The reaction was then incubated at 37° C. for 1 hour. The digested MCM184 vector backbone was then separated from the Upper MVA Pathway DNA fragment by electrophoresis on a 1.2% E-gel (Invitrogen).

The Lower MVA Pathway fragment (KKDyI) was digested sequentially with SacI and PstI restriction endonucleases from plasmid MCM107. A reaction consisting of 2 uL MCM107 (375 ng/uL), 3 uL Roche 10× Buffer A, 2 uL SacI restriction endonuclease, and 23 uL ddH₂O was prepared and incubated at 37° C. for 3 hours. The SacI restriction endonuclease was then inactivated by heating at 65° C. for 20 minutes. The DNA fragment was then purified by using a Qiagen PCR Purification column per manufacturer's protocol. The DNA fragment was then eluted from the column with a volume of 34 uL ddH₂O. The sequential digest reaction consisted of the 34 uL SacI digested eluant, 4 uL Roche 10× Buffer H, and 2 uL PstI restriction endonuclease. The reaction was incubated at 37° C. for 2 hours before being heat inactivated at 65° C. for 20 minutes. The digested KKDyI fragment was then separated from the MCM107 vector backbone by electrophoresis on a 1.2% E-gel (Invitrogen).

A ligation reaction consisting of 3 uL MCM184 vector backbone, 6 uL KKDyI DNA fragment, 2 uL New England Biolabs (NEB) 10× T4 DNA Ligase Buffer, 1 ul T4 DNA ligase, and 8 uL ddH₂O were incubated at room temperature for 20 minutes. The ligation reaction was then transformed into TOP10 chemically competent E. coli cells (Invitrogen) per manufacturer's protocol and plated on LA+50 ppm spectinomycin plates. To confirm that transformants had correct sized insert fragment, a PCR screen was performed. 50 uL ddH₂O was inoculated with individual colonies from the transformation, boiled at 95° C. for 5 minutes, and microcentrifuged for 5 minutes to pellet cellular debri. PCR was performed using PuReTaq Ready-To-Go PCR beads (GE Healthcare). Individual reaction tubes contained 1 uL of boiled cell lysate, 1 uL 10 uM primer EL-976 (SEQ ID NO:142), 1 uL 10 uM primer EL-977 (SEQ ID NO:143), and 22 uL ddH₂O. PCR tubes were cycled 1× 95° C. for 1 minute, 30× (95° C. for 30 seconds, 53° C. for 30 seconds, 72° C. for 45 seconds), 1× 72° C. for 2 minutes. The PCR products were then analyzed on a 1.2% E-gel for an 840 bp fragment. Clones #2, #3, and #4 were contained the correct sized fragments and were DNA sequenced using primers EL-976 (SEQ ID NO:142) and EL-978 (SEQ ID NO:144). DNA sequencing confirmation showed that all 3 were correct.

### Example 8

### Metabolite Analysis and Growth Inhibition

I. Metabolite Extraction from E. coli. Sampled from 14-L Fermentors.

The metabolism of bacterial cells grown in fermentors was rapidly inactivated by withdrawing approximately 4 mL of culture into a tube filled with 8 mL of dry ice-cold methanol. The resulting samples were weighed to calculate the amount of sampled broth and then put into −80° C. for storage until further analysis. For metabolite extraction and concentration, 1.5 to 4.0 mL aliquots of cell suspension were diluted with methanol/ammonium acetate buffer (5 mM, pH=8.0) mixture (6:1, v/v) to a final volume of 6 mL, and cell debris was pelleted by a 5 minute centrifugation. The supernatant was collected and loaded onto a Strata-X-AW column (Phenomenex) containing 30 mg of sorbent that selectively retains strong organic acids. The pellet was extracted two more times, first with 3 mL of the methanol/ammonium acetate buffer (5 mM, pH=8.0) mixture (6:1 v/v), and then with 6 mL of methanol/ammonium acetate buffer (5 mM, pH=8.0) mixture (1:1 v/v). Both times the cells were pelleted by centrifugation, and the resulting supernatants were consecutively loaded onto the same Strata-X-AW column. During the extraction-centrifugation, samples with cells were kept below 4° C. to minimize degradation of metabolites. After washing the columns with 1 mL of water and 1 mL of methanol, metabolites of interest were eluted from the columns first with 0.3 mL of concentrated NH₄OH/methanol (1:14, v/v) mixture and then with 0.3 mL of concentrated NH₄OH/methanol/water (1:12:2, v/v) mixture. The resulting eluant was neutralized by adding 20 μL of glacial acetic acid, and then cleared by centrifugation in a microcentrifuge.

II. Metabolite Extraction from E. coli. Grown in Shake Flasks.

To extract metabolites from shake flask-grown E. coli, methanol-quenched cells were pelleted by centrifugation, and the resulting supernatant was loaded onto Strata-X-AW anion exchange column (Phenomenex) containing 30 mg of sorbent. The pellet was re-extracted twice with several milliliters of 50%, v/v, aqueous methanol containing 20% ammonium bicarbonate buffer (pH=8.0) and then with 75%, v/v, aqueous bicarbonate-buffered methanol. After each extraction, cell debris was pelleted by centrifugation, and the supernatant was consecutively loaded onto the same anion exchange columns. During the extraction and centrifugation steps, the samples were kept at below +4° C. Prior to metabolite elution, the columns were washed with water and methanol (1 mL of each), and the analytes were eluted by adding 0.3 mL of concentrated NH₄OH/methanol (1:14, v/v) and then 0.3 mL of concentrated NH₄OH/water/methanol (1:2:12) mixtures. The eluant was neutralized with 40 μL of glacial acetic acid and then cleared by centrifugation in a microcentrifuge.

### III. Metabolite Quantification

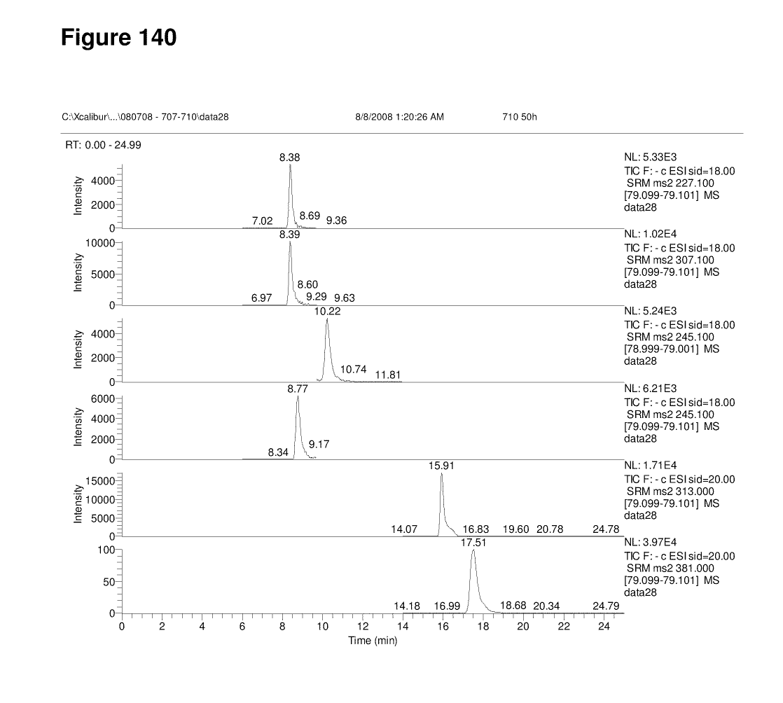

Analysis of metabolites was carried out using a Thermo Finnigan TSQ system (Thermo Electron Corporation, San Jose, Calif.). All system control, data acquisition, and mass spectral data evaluation were performed using XCalibur and LCQuan software (Thermo Electron Corp). For the LC-ESI-MS/MS method, a chiral Nucleodex β-OH 5 μM HPLC column (200×4 mm, Macherey-Nagel, Germany) was used with a CC 8/4 Nucleodex beta-OH guard cartridge. A mobile phase gradient (Table 9) was applied at a flow rate of 0.8 mL/min in which mobile phase A was MilliQ—grade water, mobile phase B was 100 mM ammonium acetate (SigmaUltra grade, Sigma) buffer (pH adjusted to 8.0 by ammonium hydroxide) in MilliQ—grade water and mobile phase C was LC-MS grade acetonitrile (Chromasolv, Riedel-de Haën). The column and sample tray temperatures were reduced to 5° C. and 4° C., respectively. The injection volume was 10 or 20 μL. FIG. 140 shows typical elution profiles of selected metabolites extracted from an isoprene-producing E. coli strain.

TABLE 9
HPLC gradient used to elute metabolites in the MVA pathway.

| | Mobile phase, % | | |
|---|---|---|---|
| | | B | |
| | | (100 mM | |
| Time, | A | ammonium | C |
| min | (water) | acetate, pH = 8.0) | (acetonitrile) |
| 0.0 | 0.0 | 20.0 | 80.0 |
| 1.0 | 0.0 | 20.0 | 80.0 |
| 8.0 | 0.0 | 50.0 | 50.0 |
| 11.0 | 0.0 | 50.0 | 50.0 |
| 13.0 | 46.0 | 4.0 | 50.0 |
| 19.0 | 49.6 | 0.4 | 50.0 |
| 22.5 | 49.6 | 0.4 | 50.0 |
| 23.0 | 0.0 | 20.0 | 80.0 |
| 25.0 | 0.0 | 20.0 | 80.0 |

Mass detection was carried out using electrospray ionization in the negative mode (ESI spray voltage of 2.5-3.0 kV and ion transfer tube temperature of 390° C.). The following m/z values for precursor ions were selected to detect the metabolites of interest in SRM mode: 245.0 for IPP and DMAPP, 313.1 for GPP, 381.1 for FPP, 227.0 for MVP, and 307.1 for MVPP. Concentrations of metabolites were determined based on the integrated intensities of peaks generated by PO₃⁻ product ion (m/z=79.0). Calibration curves obtained by injection of standards (IPP, DMAPP, and GPP purchased from Sigma-Aldrich, and FPP purchased from Echelon Biosciences Inc.) were used to calculate concentrations of metabolites in cell extracts. Concentrations of MVP and MVPP were expressed in arbitrary units because of the absence of commercially available standards. Intracellular concentrations of metabolites were determined based on the assumption that in 1 mL of the culture at OD=200 the integrated volume of all cells is 50 μL.

IV. Intracellular Concentrations of Metabolites in the MCM401 Strain of E. coli Containing MVK from M. mazei Under Different Levels of Enzyme Expression Induced by Adding IPTG to the Fermentors.

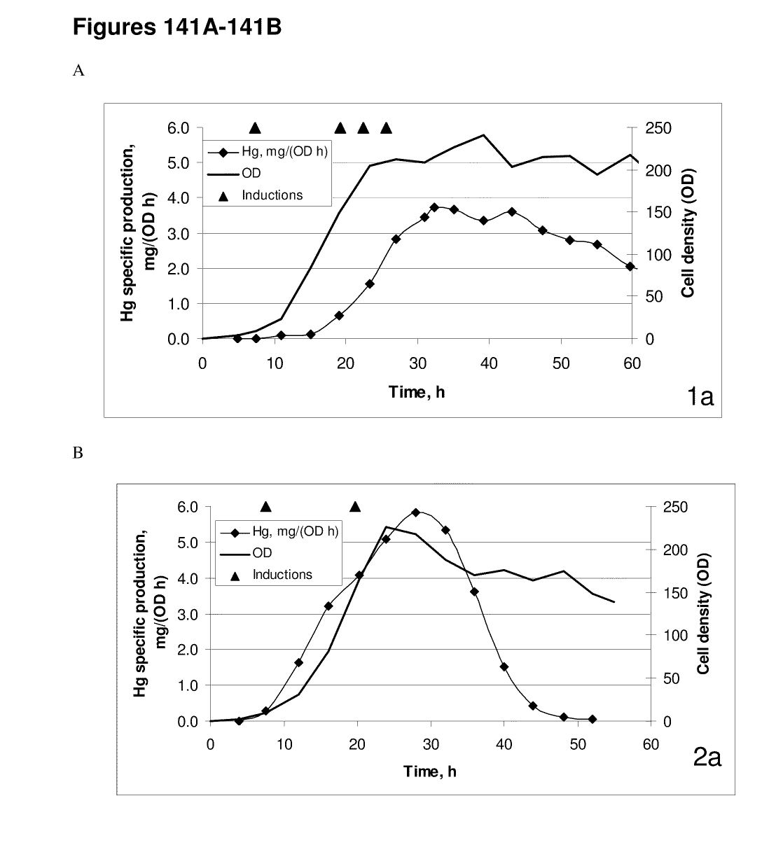

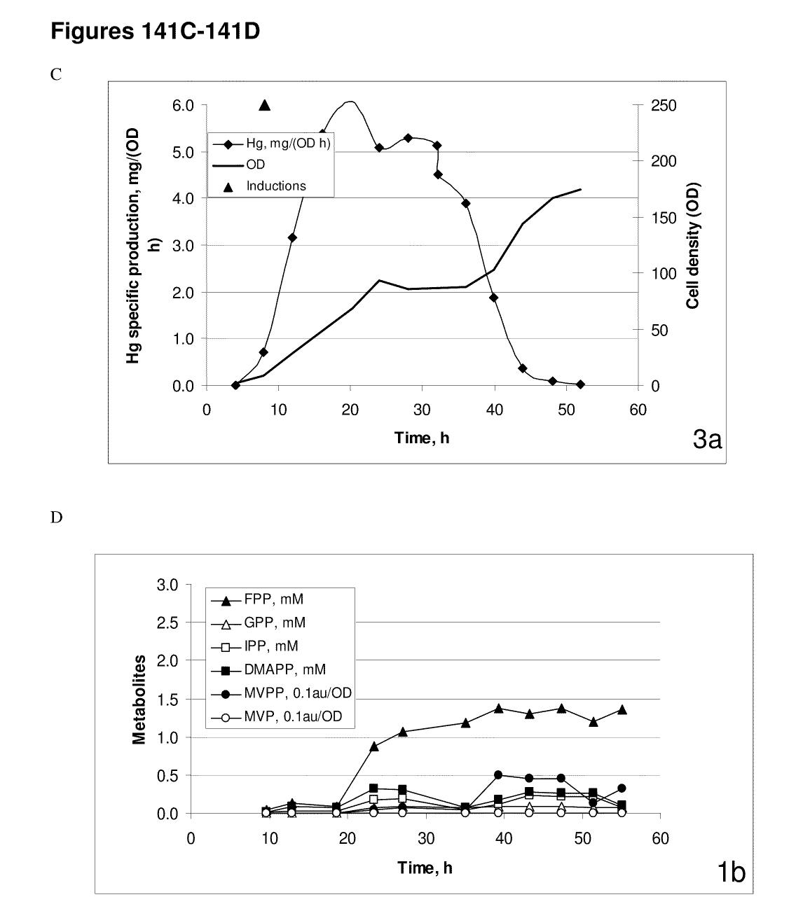

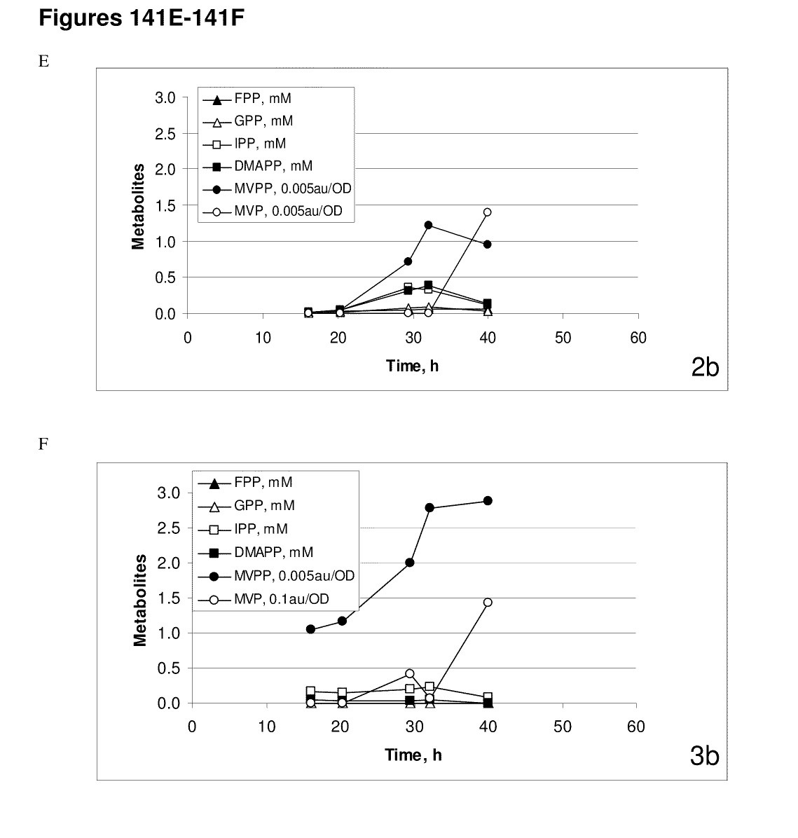

FIG. 141A-141F provide an example of intracellular concentrations of metabolites in the MCM401 strain of E. coli containing MVK from M. mazei under different levels of enzyme expression induced by adding IPTG to the fermentors. Even though the final IPTG concentrations in all three fermentors were similar (˜200 μM), cell response was very different depending on the IPTG feeding scheme. A single-shot addition of a high dose of IPTG (FIGS. 114C and 114F) caused an instant increase in isoprene production and early accumulation of a significant level of MVPP. In contrast, concentrations of DMAPP, the immediate precursor of isoprene, as well as GPP and FPP, the products of IPP and DMAPP condensation, were low (below ˜0.2 mM). Intracellular concentrations of IPP remained higher than the concentration of DMAPP during the analyzed fermentation period, indicating that DMAPP is synthesized from IPP slower than it is consumed in the isoprene biosynthesis reaction.

Although the maximum specific productivity of MCM401 cells reached about the same level upon adding IPTG in two steps (˜100 μM each time; FIGS. 141B and 141E), the amount of MVPP accumulated in cells by the end of the production period was lower than in the single IPTG shot experiment and the buildup of MVPP pool started only after the second portion of IPTG was added to the fermentor. In both cases a decline in the isoprene production correlated with accumulation of MVP, which pool reached much higher concentrations in cells that had received two doses of IPTG. Moderate levels of IPP and DMAPP (˜0.4 mM) were detected in the latter case around 30 hours of fermentation, which correlated in time with the maximum rate of isoprene biosynthesis by these cells. Notably, intracellular concentrations of GPP and FPP were low presumably due to a very high activity of the isoprene synthase.

Four IPTG shots of about 50 μM each resulted in the lowest specific productivity of the MCM401 strain; however, under these conditions the culture continued to synthesize isoprene at a significant rate for a longer period of time (FIGS. 141A and 141D). The maximum intracellular levels of IPP and DMAPP generally remained in the range of 0.2-0.4 mM during the production period, and FPP raised to 1.0-1.5 mM in response to the second 50 μM dose of IPTG. Notably, DMAPP concentration was slightly higher than the concentration of IPP likely due to the fact that DMAPP conversion into isoprene occurred slower in this case compared to the fermentations illustrated in FIGS. 141B, 141C, 141E, and 141F, and FPP biosynthesis did not consume significant amounts of DMAPP.

V. Intracellular Concentrations of Metabolites in the MCM402 Strain of E. coli Overexpressing MVK from Saccharomyces cerevisiae

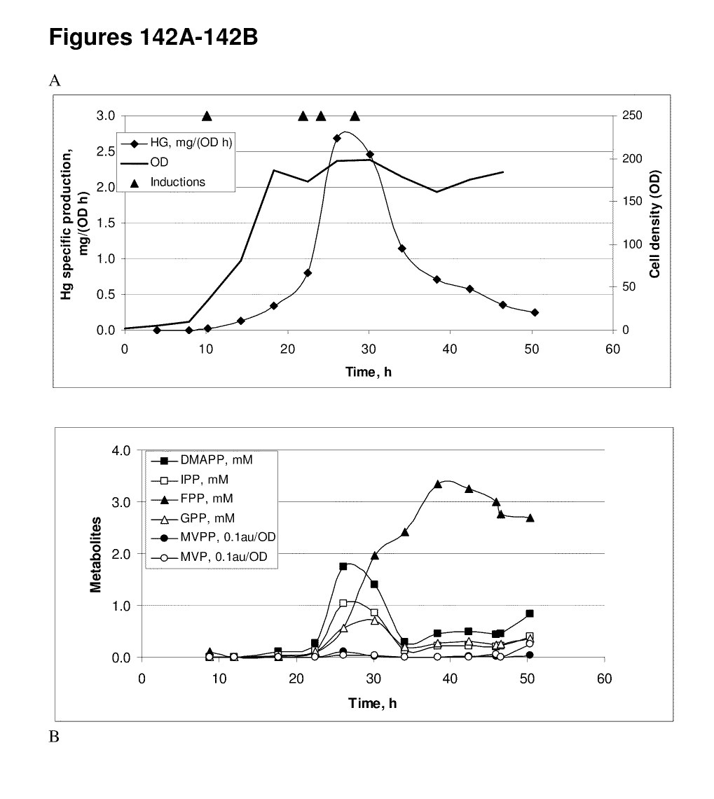

FIGS. 142A and 142B illustrate the experiment with the MCM402 strain of E. coli, containing overexpressed MVK from Saccharomyces cerevisiae. As in the case with the MCM401 strain having MVK from M. mazei and grown under similar IPTG induction conditions (4×50 μM shots), isoprene production started after the second dose of IPTG has been added to the fermentor, which coincided in time with rapid accumulation of DMAPP and IPP to relatively high levels (up to 1.8 mM of DMAPP) in the MCM402 cells. However, in the MCM402 cells, the isoprene production period remained very short correlating with the drop in DMAPP and IPP pools. In contrast, FPP continued to accumulate up to the level of 2.6-3.5 mM even when DMAPP and IPP concentrations dropped to below 1 mM.

VI. Intracellular Concentrations of Metabolites in the MCM402 Strain of E. coli Overexpressing MVK from Streptomyces

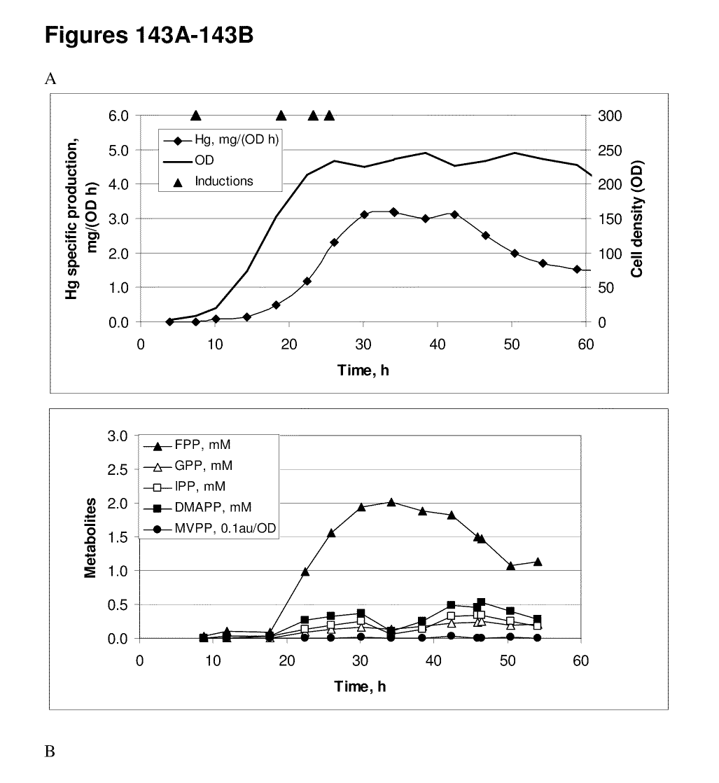

FIGS. 143A and 143B illustrate the experiment with the MCM400 strain of E. coli, containing overexpressed MVK from Streptomyces. In terms of accumulation of isoprenoid intermediates/precursors and isoprene production results of this experiment are very similar to the experiment performed with the MCM401 strain containing MVK from M. mazei and induced with IPTG using the same scheme (4×50 μM shots; see FIGS. 141A and 141D). Indeed, the isoprene specific productivity in the MCM400 strain reached values slightly above 3 mg/(OD h), and the high rate of production was maintained for a long time. Moreover, MCM400 cells accumulated up to 2 mM of FPP with the FPP accumulation started after the second IPTG shot; DMAPP, IPP, and GPP concentrations remained within the range of 0.2-0.5 mM during the production period, and MVP and MVPP were below the detection limit. Therefore, parts IV to VI of this example emphasize superior properties of MVK from Streptomyces and M. mazei as compared to yeast MVK.

VII. Safe and Maximal Metabolite Concentrations During Isoprene Production Shake Flask Experiment with MCM127

A shake flask experiment with MCM127 was performed to investigate the accumulation of key intermediates during strong induction of isoprene production. Strong induction of this strain resulted in growth inhibition most likely due to accumulation of toxic metabolic intermediates.

### Medium Recipe (Per Liter Fermentation Medium):

Each liter of fermentation medium contained K₂HPO₄ 13.6 g, KH₂PO₄ 13.6 g, MgSO₄* 7H₂O 2 g, citric acid monohydrate 2 g, ferric ammonium citrate 0.3 g, (NH₄)₂SO₄ 3.2 g, yeast extract 1 g, 1000× Trace Metal Solution 1 ml. All of the components were added together and dissolved in diH₂O. The pH was adjusted to 6.8 with ammonium hydroxide (30%) and brought to volume. Medium was filter-sterilized with a 0.22 micron vacuum filter. Glucose was added to the medium to a final concentration of 0.5%. Antibiotics were added after sterilization and pH adjustment.

### 1000× Trace Metal Solution (Per Liter Fermentation Medium):

1000× trace metal solution contained citric Acids*H₂O 40 g, MnSO₄*H₂O 30 g, NaCl 10 g, FeSO₄*7H₂O 1 g, CoCl₂*6H₂O 1 g, ZnSO₄*7H₂O 1 g, CuSO₄*5H₂O 100 mg, H₃BO₃ 100 mg, NaMoO₄*2H₂O 100 mg. Each component was dissolved one at a time in diH₂O, pH to 3.0 with HCl/NaOH, and then brought to volume and filter sterilized with 0.22 micron filter.

### Strain:

The MCM127 strain is BL21 (DE3) E. coli cells containing the upper mevalonic acid (MVA pathway (pCL Upper) and the lower MVA pathway including isoprene synthase from kudzu (pTrcKKDyIkIS)

An inoculum of E. coli strain MCM127 taken from a frozen vial was streaked onto an LB broth agar plate (with antibiotics) and incubated at 30° C. A single colony was inoculated into media containing glucose as carbon source and grown overnight at 30° C. The bacteria were diluted into fermentation media to reach an optical density of 0.05 measured at 550 nm. A total of 150 mL of culture was dispensed into two 500 mL flasks that were then shaken at 170 rpm in a 30° C. incubator. When the cultures reached an optical density (OD₆₀₀) of 0.5, one of the flasks was induced with 150 μM isopropyl-beta-D-1-thiogalactopyranoside (IPTG). Samples of 20 mL from both the induced and non-induced culture were taken approximately every half hour for metabolite analysis after induction. The samples were quickly quenched in equal volume of methanol cooled on dry ice. After centrifugation, supernatant was loaded on Stata X-AW columns. The pellet was resuspended in 5 mL of Methanol-water (6:1, water contained 5 mM NH4Ac at pH=8.0), cell debris were separated by centrifugation, and the supernatant was loaded on the Stata X-AW columns. Metabolites were eluted with 0.30 mL ethanol:conc NH4OH (14:1 vol/vol), then with 0.3 mL methanol:water:conc NH4OH (12:2:1 vol/vol/vol), finally pH was adjusted by adding 40 uL of glacial acetic acid. Extracted metabolites were analyzed by LCMS using a standard cyclodextrin column protocol. To increase sensitivity, only ions corresponding to IPP, DMAPP, GPP, and FPP were detected. Injection volume was 20 uL/sample. Standards of all metabolites were used for calibration.

Upon induction of the MCM127 with 150 μM IPTG, the bacteria continued to grow identical to the un-induced strain for approximately one and a half hour. After this, the induced culture began to show signs of growth inhibition (FIG. 112A). Key metabolites were measured during the experiment and showed an increasing accumulation of FPP, GPP, DMAPP and IPP after induction. DMAPP and IPP only began to accumulate when the induced bacteria first showed signs of growth inhibition (FIG. 112B). None of the mentioned intermediates were detected in measurable amount in the un-induced culture. The experiment demonstrates that E. coli can tolerate significant intracellular concentrations of GPP and FPP (Tables 15A and 15B), while accumulation of DMAPP and IPP coincides with growth inhibition when cultures are grown in shake flasks. Data in Tables 15A and 15B were from the 5.5 hr time point, where growth was still normal in the induced culture.

VIII. Intracellular Concentrations of Metabolites in the MCM343 Strain of E. coli Expressing the Full Mevalonic Acid Pathway and Kudzu Isoprene Synthase (Without Overexpression of a Second Mevalonate Kinase)

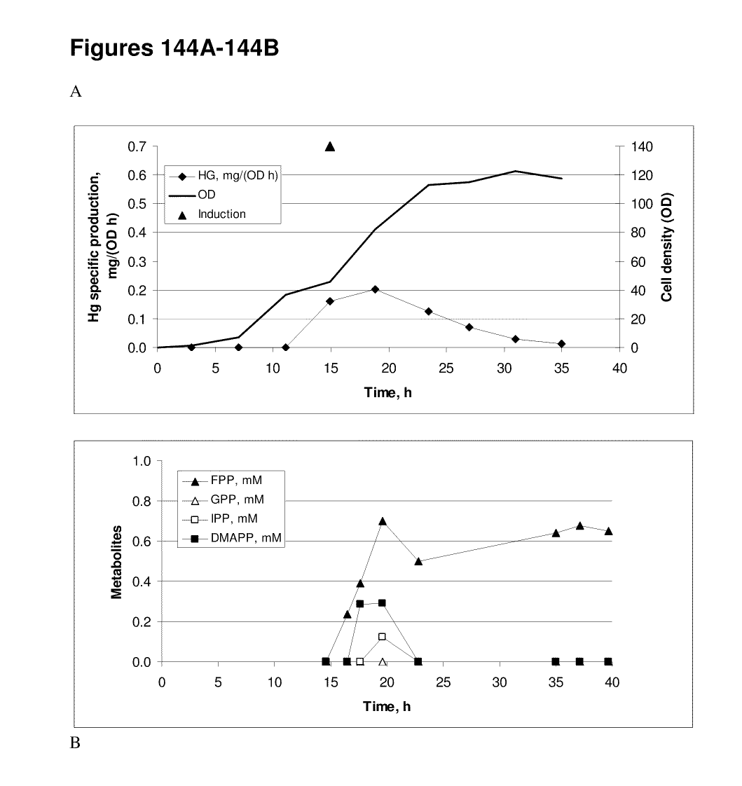

FIGS. 144A and 144B depict changes in concentrations of selected intermediates in the isoprenoid pathway in the course of fermentation of MCM343 E. coli strain. This fermentation run was characterized by very low specific productivity and barely detectable concentrations of most of isoprenoid intermediates except for FPP, which intracellular level reached 0.7 mM, after 100 μM IPTG was added to the cells. IPP and DMAPP were detected shortly after the IPTG addition and then their level dropped below the detection limit. No MVP or MVPP were detected during the fermentation.

### IX. Growth Inhibition

i) Recovery of Mevalonic Acid from fermentation Broth.

Mevalonic acid was obtained by a fed batch fermentation of Escherichia coli strain, BL21 harboring an expression plasmid bearing the genes mvaS and mvaE from Enterococcus faecalis (U.S. Appl. Pub. No. 2005/0287655, which is incorporated by reference in its entirety, particularly with respect to genes mvaS and mvaE). Fermentation of the strains was carried out in fed batch fermentation mode in a minimal medium with a glucose feed for 40 hours. Broth was harvested, mixed with diatomaceous earth (DE; Catalog #Celatom FW-12, American Tartaric Products Inc.), and filtered under vacuum through a Buchner funnel fitted with a filter pad. The filtrate was sterile filtered through a 10,000 MWCO membrane. Mevalonic acid was converted to the lactone by acidification and recovered by continuous organic solvent extraction; NMR analysis indicated a purity of 84%. All recovery steps are well known to those skilled in the art. When the free acid was required for experiments, the MVA lactone was hydrolyzed by the addition of 1 equivalent of base to a solution of lactone and allowed to stand for 1 hour prior to use. The sterile filtered solution can be stored for extended time at 4° C.

ii) Growth Inhibition of Escherichia coli BL21 by the Accumulation of Mevalonate Diphosphate, Isopentenyl Diphosphate (IPP), and Dimethylallyl Diphosphate (DMAPP).

The purpose of this experiment was to determine the effect of the expression of the proteins mevalonate kinase (MVK), phophomevalonate kinase (PMK), and diphosphomevalonate decarboxylase (MDD) of Escherichia coli cultures.

E. coli BL21 cells bearing pTrcK, representing a plasmid expressing MVK, pTrcKK representing a plasmid expressing MVK plus PMK, and pTrcKKD, representing a plasmid expressing MVK plus PMK plus MDD were grown at approximately 30° C. and 250 rpm in 250 mL flasks containing 25 mL of TM3 medium (13.6 g K₂PO₄, 13.6 g KH₂PO₄, 2.0 g MgSO₄*7H₂O) supplemented with 1% glucose and 0.8 g/L Biospringer yeast extract (1% Yeast extract final). When OD600 reached 0.8 to 0.9, 5.8 mM mevalonic acid was added to the cultures and incubation was continues for an additional 5 hours. OD₆₀₀ measurements were taken, and the cultures were sampled for metabolite analysis at 2 hours post MVA addition. Samples were collected into 100% MeOH prechilled in dry ice in a ratio of 1:1. Samples were stored at −80° C. until analyzed as follows. The methanol-quenched cells were pelleted by centrifugation and the resulting supernatant was loaded onto Strata-X-AW anion exchange column (Phenomenex) containing 30 mg of sorbent. The pellet was reextracted twice with several milliliters of 50%, v/v, aqueous methanol containing 20% ammonium bicarbonate buffer (pH=8.0) and then with 75%, v/v, aqueous bicarbonate-buffered methanol. After each extraction, cell debris were pelleted by centrifugation and the supernatant was consecutively loaded onto the same anion exchange columns. During the extraction and centrifugation steps, the samples were kept at below +4° C. Prior to metabolite elution, the columns were washed with water and methanol (1 mL of each) and the analytes were eluted by adding 0.3 mL of concentrated NH₄OH/methanol (1:14, v/v) and then 0.3 mL of concentrated NH₄OH/water/methanol (1:2:12) mixtures. The eluant was neutralized with 40 μL of glacial acetic acid and then cleared by centrifugation in microcentrifuge. Analysis of metabolites in these samples is as described above.

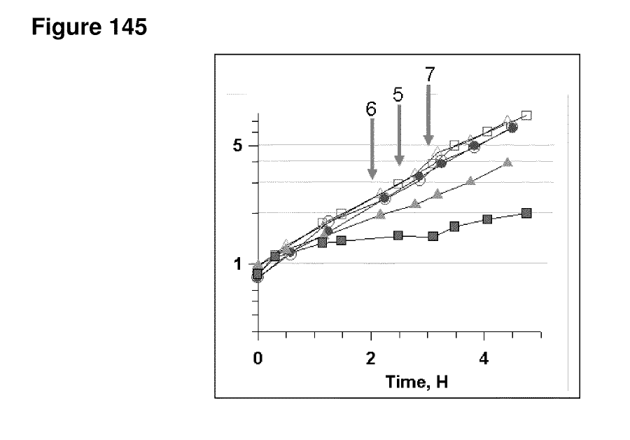

As is shown in FIG. 145, inhibition of growth was evident when the enzymes MVK and PMK are expressed (strain #7); additional inhibition is observed when MDD is added to the cloned pathway (strain #6). No growth inhibition was observed when MVK was the only enzyme expressed (strain #5). Analysis of MVA concentration at the time of collection of samples suggests that strain with MVK plus PMK plus MDD consumed 2.9 mM MVA while the other two strains consume lower quantities. Measurement of phosphomevalonate from the culture of the strains carrying only MVK was not successful; however, the culture carrying MVK and PMV showed about 30 and 60-fold higher levels, respectively, of phosphomevalonate and diphosphomevalonate compared to the strain carrying MVK, PMK, and MDD. The latter strain accumulated surprisingly high levels of IPP and DMAPP on the order of 40 mM IPP and 320 uM DMAPP when calculated as an intracellular concentration. These measurements were conducted on whole cell broth; thus, some of the metabolites may have been excreted by the cells. While not intending to be bound by any particular theory, it is believed that the observed growth inhibition is due to the accumulation of one or more of these metabolites. A goal is therefore to achieve a pathway enzyme balance to minimize the accumulation of these metabolites for the relief of growth inhibition.

### Example 9

### Production of Isoprene by E. coli Expressing the Upper Mevalonic Acid (MVA) Pathway, the Integrated Lower MVA Pathway (gi1.2KKDyI), Mevalonate Kinase from Streptomyces, and Isoprene Synthase from Kudzu and Grown in Fed-Batch Culture at the 15-L Scale

### Medium Recipe (Per Liter Fermentation Medium):

Each liter of fermentation medium contained K₂HPO₄ 7.5 g, MgSO₄*7H₂O 2 g, citric acid monohydrate 2 g, ferric ammonium citrate 0.3 g, yeast extract 0.5 g, and 1000× Modified Trace Metal Solution 1 ml. All of the components were added together and dissolved in diH₂O. This solution was autoclaved. The pH was adjusted to 7.0 with ammonium hydroxide (30%) and q.s. to volume. Glucose 10 g, thiamine*HCl 0.1 g, and antibiotics were added after sterilization and pH adjustment.

### 1000× Modified Trace Metal Solution:

1000× Modified Trace Metal Solution contained citric Acids*H₂O 40 g, MnSO₄*H₂O 30 g, NaCl 10 g, FeSO₄*7H₂O 1 g, CoCl₂*6H₂O 1 g, ZnSO₄*7H₂O 1 g, CuSO₄*5H₂O 100 mg, H₃BO₃ 100 mg, and NaMoO₄*2H₂O 100 mg. Each component was dissolved one at a time in DI H₂O, pH to 3.0 with HCl/NaOH, then q.s. to volume and filter sterilized with a 0.22 micron filter.

Fermentation was performed in a 15-L bioreactor with BL21 (DE3) E. coli cells containing the upper mevalonic acid (MVA) pathway (pCL PtrcUpperPathway encoding E. faecalis mvaE and mvaS), the integrated lower MVA pathway (gi1.2KKDyI encoding S. cerevisiae mevalonate kinase, mevalonate phosphate kinase, mevalonate pyrophosphate decarboxylase, and IPP isomerase), and high expression of mevalonate kinase from Streptomyces CL190 and isoprene synthase from Kudzu (pTrcKudzuMVK(StreptomycesCL190)). This experiment was carried out to monitor isoprene formation from glucose at the desired fermentation pH 7.0 and temperature 30° C. An inoculum of E. coli strain taken from a frozen vial was streaked onto an LB broth agar plate (with antibiotics) and incubated at 37° C. A single colony was inoculated into tryptone-yeast extract medium. After the inoculum grew to OD 1.0, measured at 550 nm, 500 mL was used to innoculate 5-L of cell medium in the 15-L bioreactor. The liquid volume increases throughout the fermentation (such as to approximately 10 liters).

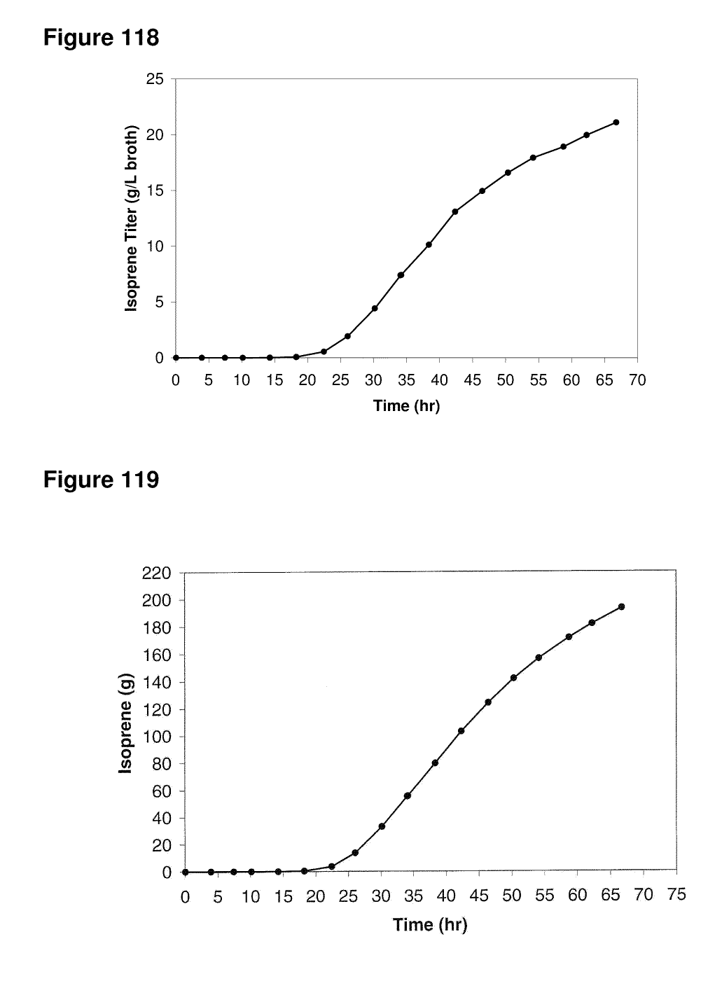

Glucose was fed at an exponential rate until cells reached the stationary phase. After this time the glucose feed was decreased to meet metabolic demands. The total amount of glucose delivered to the bioreactor during the 67 hour fermentation was 3.5 kg. Induction was achieved by adding IPTG. The IPTG concentration was brought to 50 uM when the optical density at 550 nm (OD₅₅₀) reached a value of 9. The IPTG concentration was raised to 88 uM when OD₅₅₀ reached 165. Additional IPTG additions raised the concentration to 114 uM at OD₅₅₀=215 and 147 uM at OD₅₅₀=230. The OD₅₅₀ profile within the bioreactor over time is shown in FIG. 117. The isoprene level in the off gas from the bioreactor was determined using a Hiden mass spectrometer. The isoprene titer increased over the course of the fermentation to a final value of 21.1 g/L (FIG. 118). The total amount of isoprene produced during the 67 hour fermentation was 193.2 g and the time course of production is shown in FIG. 119. The molar yield of utilized carbon that went into producing isoprene during fermentation was 12.0%. The weight percent yield of isoprene from glucose was 6.2%.

### Example 10

### Production of Isoprene by E. coli Expressing the Upper Mevalonic Acid (MVA) Pathway, the Integrated Lower MVA Pathway (gi1.2KKDyI), Mevalonate Kinase from Lactobacillus, and Isoprene Synthase from Kudzu and Grown in Fed-Batch Culture at the 15-L Scale

### Medium Recipe (Per Liter Fermentation Medium):

Each liter of fermentation medium contained K₂HPO₄ 7.5 g, MgSO₄*7H₂O 2 g, citric acid monohydrate 2 g, ferric ammonium citrate 0.3 g, yeast extract 0.5 g, and 1000× Modified Trace Metal Solution 1 ml. All of the components were added together and dissolved in diH₂O. This solution was autoclaved. The pH was adjusted to 7.0 with ammonium hydroxide (30%) and q.s. to volume. Glucose 10 g, thiamine*HCl 0.1 g, and antibiotics were added after sterilization and pH adjustment.

### 1000× Modified Trace Metal Solution:

1000× Modified Trace Metal Solution contained citric Acids*H₂O 40 g, MnSO₄*H₂O 30 g, NaCl 10 g, FeSO₄*7H₂O 1 g, CoCl₂*6H₂O 1 g, ZnSO₄*7H₂O 1 g, CuSO₄*5H₂O 100 mg, H₃BO₃ 100 mg, and NaMoO₄*2H₂O 100 mg. Each component was dissolved one at a time in DI H₂O, pH to 3.0 with HCl/NaOH, then q.s. to volume and filter sterilized with a 0.22 micron filter.

Fermentation was performed in a 15-L bioreactor with BL21 (DE3) E. coli cells containing the upper mevalonic acid (MVA) pathway (pCL PtrcUpperPathway encoding E. faecalis mvaE and mvaS), the integrated lower MVA pathway (gi1.2KKDyI encoding S. cerevisiae mevalonate kinase, mevalonate phosphate kinase, mevalonate pyrophosphate decarboxylase, and IPP isomerase), and high expression of mevalonate kinase from Lactobacillus and isoprene synthase from Kudzu (pTrcKudzuMVK(Lactobacillus)). This experiment was carried out to monitor isoprene formation from glucose at the desired fermentation pH 7.0 and temperature 30° C. An inoculum of E. coli strain taken from a frozen vial was streaked onto an LB broth agar plate (with antibiotics) and incubated at 37° C. A single colony was inoculated into tryptone-yeast extract medium. After the inoculum grew to OD 1.0, measured at 550 nm, 500 mL was used to innoculate 5-L of cell medium in the 15-L bioreactor. The liquid volume increases throughout the fermentation (such as to approximately 10 liters).

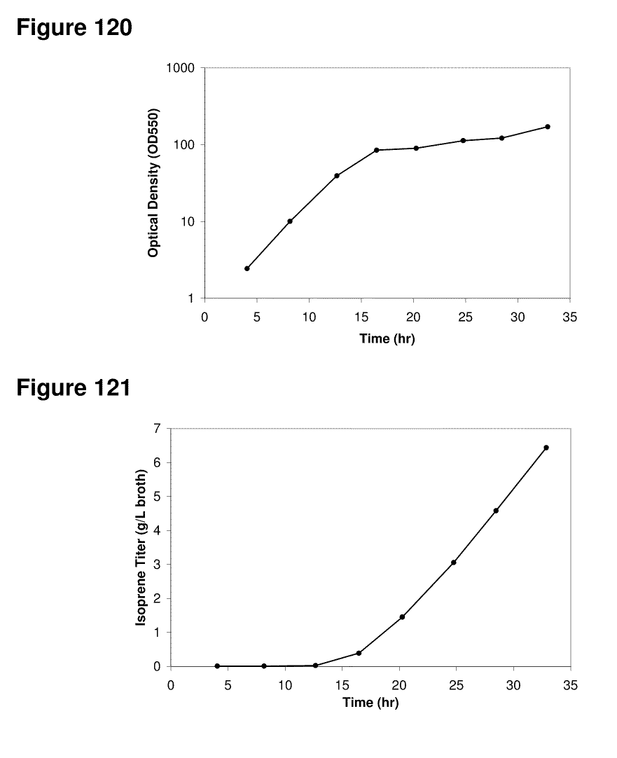

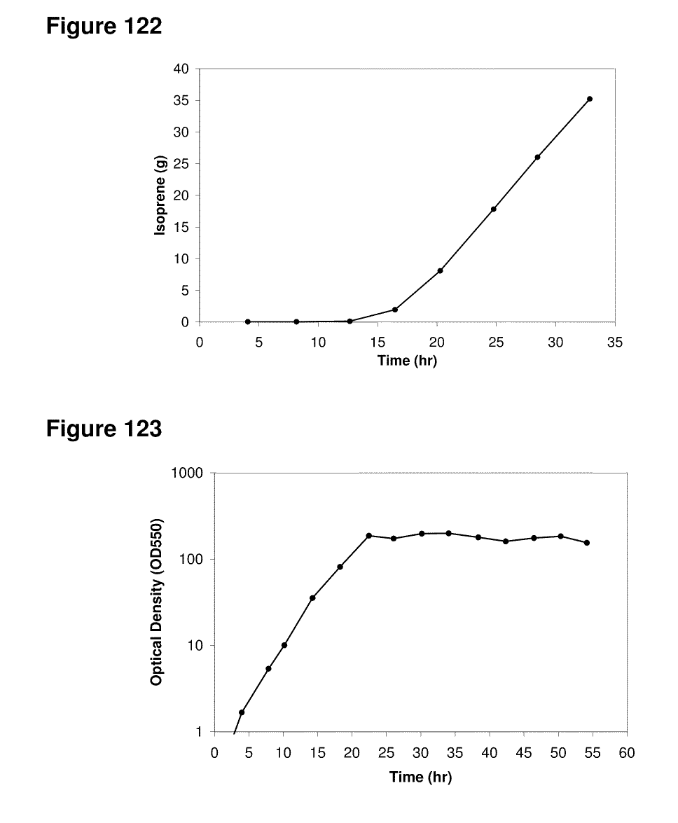

Glucose was fed at an exponential rate until cells reached the stationary phase. After this time the glucose feed was decreased to meet metabolic demands. The total amount of glucose delivered to the bioreactor during the 33 hour fermentation was 1.0 kg. Induction was achieved by adding IPTG. The IPTG concentration was brought to 58 uM when the optical density at 550 nm (OD₅₅₀) reached a value of 16. The IPTG concentration was raised to 108 uM when OD₅₅₀ reached 30. Additional IPTG additions raised the concentration to 174 uM at OD₅₅₀=56 and 222 uM at OD₅₅₀=86. The OD₅₅₀ profile within the bioreactor over time is shown in FIG. 120. The isoprene level in the off gas from the bioreactor was determined using a Hiden mass spectrometer. The isoprene titer increased over the course of the fermentation to a final value of 6.4 g/L (FIG. 121). The total amount of isoprene produced during the 33 hour fermentation was 35.2 g and the time course of production is shown in FIG. 122. The molar yield of utilized carbon that went into producing isoprene during fermentation was 7.2%. The weight percent yield of isoprene from glucose was 3.4%.

### Example 11

### Production of Isoprene by E. coli Expressing the Upper Mevalonic Acid (MVA) Pathway, the Integrated Lower MVA Pathway (gi1.2KKDyI), Mevalonate Kinase from Yeast, and Isoprene Synthase from Kudzu and Grown in Fed-Batch Culture at the 15-L Scale

### Medium Recipe (Per Liter Fermentation Medium):

Each liter of fermentation medium contained K₂HPO₄ 7.5 g, MgSO₄*7H₂O 2 g, citric acid monohydrate 2 g, ferric ammonium citrate 0.3 g, yeast extract 0.5 g, and 1000× Modified Trace Metal Solution 1 ml. All of the components were added together and dissolved in diH₂O. This solution was autoclaved. The pH was adjusted to 7.0 with ammonium hydroxide (30%) and q.s. to volume. Glucose 10 g, thiamine*HCl 0.1 g, and antibiotics were added after sterilization and pH adjustment.

### 1000× Modified Trace Metal Solution:

1000× Modified Trace Metal Solution contained citric Acids*H₂O 40 g, MnSO₄*H₂O 30 g, NaCl 10 g, FeSO₄*7H₂O 1 g, CoCl₂*6H₂O 1 g, ZnSO₄*7H₂O 1 g, CuSO₄*5H₂O 100 mg, H₃BO₃ 100 mg, and NaMoO₄*2H₂O 100 mg. Each component was dissolved one at a time in DI H₂O, pH to 3.0 with HCl/NaOH, then q.s. to volume and filter sterilized with a 0.22 micron filter.

Fermentation was performed in a 15-L bioreactor with BL21 (DE3) E. coli cells containing the upper mevalonic acid (MVA) pathway (pCL PtrcUpperPathway encoding E. faecalis mvaE and mvaS), the integrated lower MVA pathway (gi1.2KKDyI encoding S. cerevisiae mevalonate kinase, mevalonate phosphate kinase, mevalonate pyrophosphate decarboxylase, and IPP isomerase), and high expression of mevalonate kinase from yeast and isoprene synthase from Kudzu (pTrcKudzuMVK(yeast)). This experiment was carried out to monitor isoprene formation from glucose at the desired fermentation pH 7.0 and temperature 30° C. An inoculum of E. coli strain taken from a frozen vial was streaked onto an LB broth agar plate (with antibiotics) and incubated at 37° C. A single colony was inoculated into tryptone-yeast extract medium. After the inoculum grew to OD 1.0, measured at 550 nm, 500 mL was used to innoculate 5-L of cell medium in the 15-L bioreactor. The liquid volume increases throughout the fermentation (such as to approximately 10 liters).

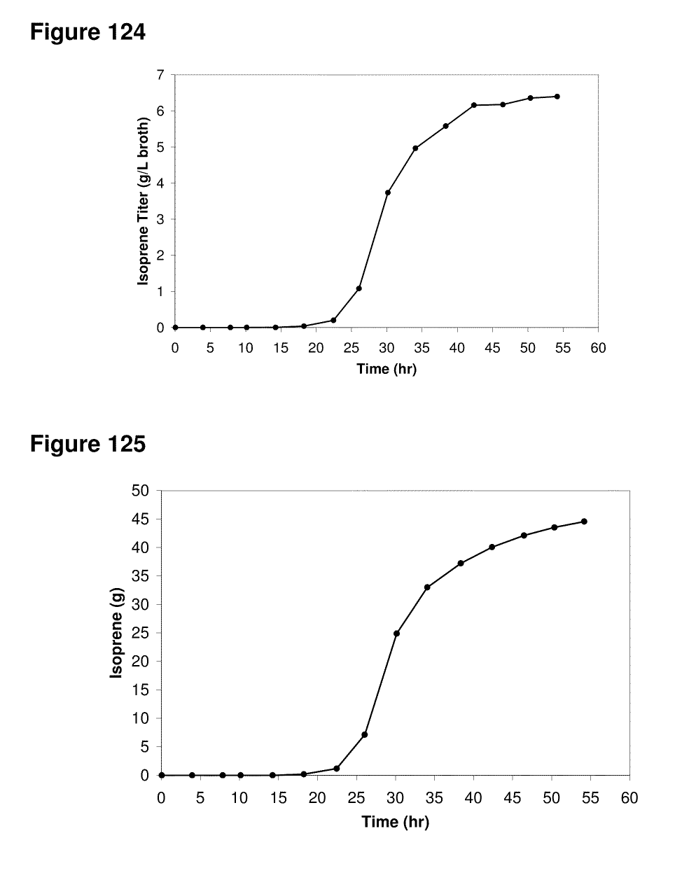

Glucose was fed at an exponential rate until cells reached the stationary phase. After this time the glucose feed was decreased to meet metabolic demands. The total amount of glucose delivered to the bioreactor during the 54 hour fermentation was 1.6 kg. Induction was achieved by adding IPTG. The IPTG concentration was brought to 54 uM when the optical density at 550 nm (OD₅₅₀) reached a value of 10. The IPTG concentration was raised to 87 uM when OD₅₅₀ reached 175. Additional IPTG additions raised the concentration to 122 uM at OD₅₅₀=180 and 157 uM at OD₅₅₀=185. The OD₅₅₀ profile within the bioreactor over time is shown in FIG. 123. The isoprene level in the off gas from the bioreactor was determined using a Hiden mass spectrometer. The isoprene titer increased over the course of the fermentation to a final value of 6.4 g/L (FIG. 124). The total amount of isoprene produced during the 54 hour fermentation was 44.6 g and the time course of production is shown in FIG. 125. The molar yield of utilized carbon that went into producing isoprene during fermentation was 6.1%. The weight percent yield of isoprene from glucose was 2.8%.

### Example 12

### Construction and Expression of Lactobacillus sakei and Streptococcus pneumoniae Mevalonate Kinase Constructs

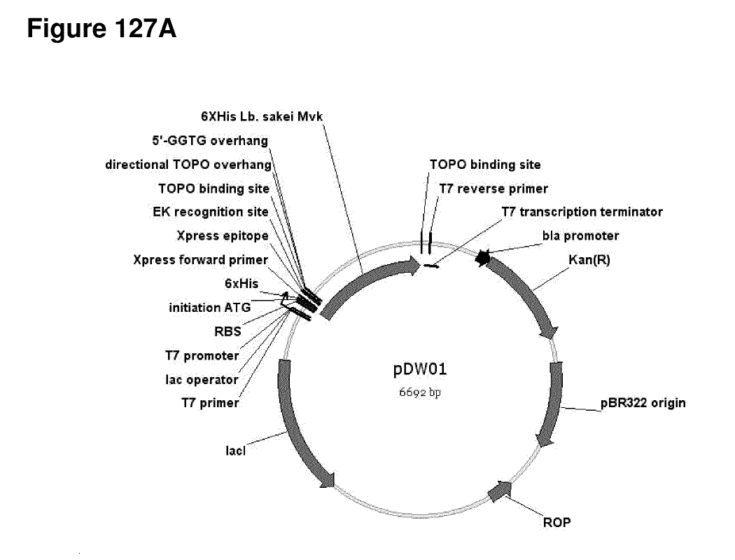

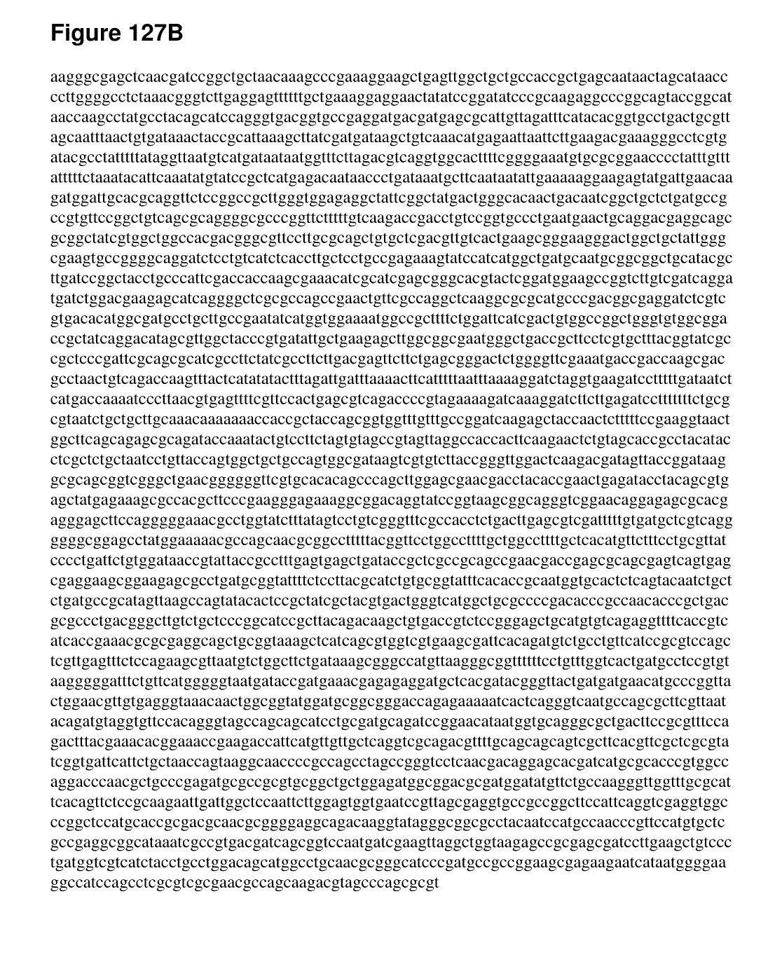

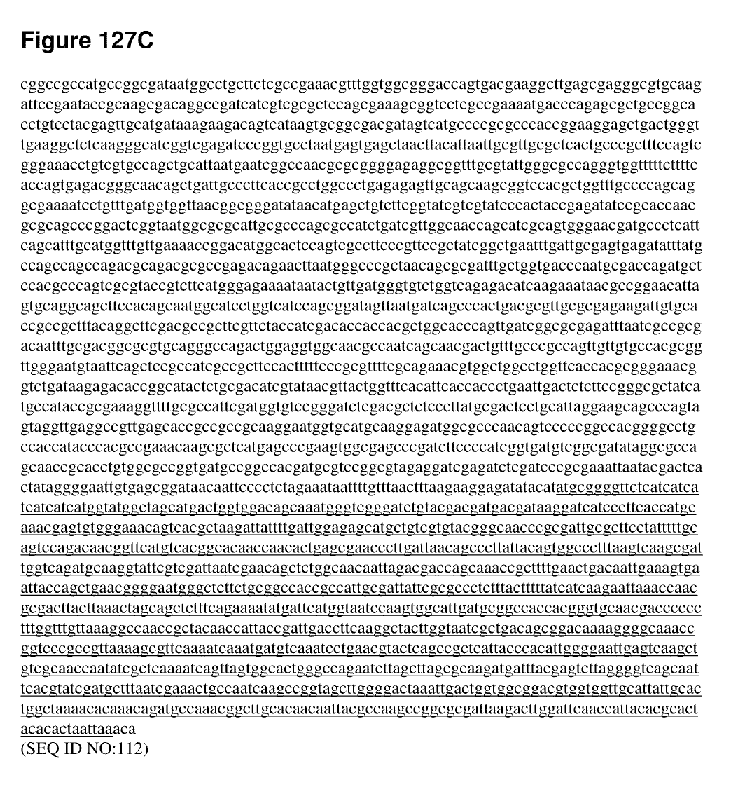

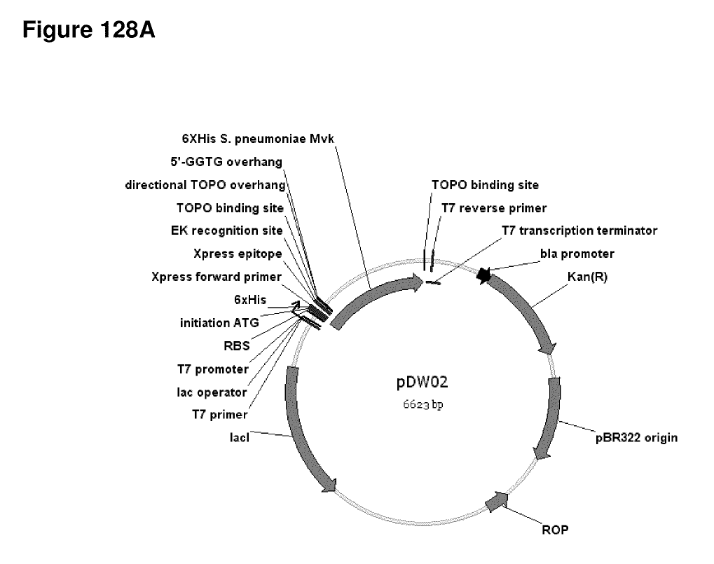

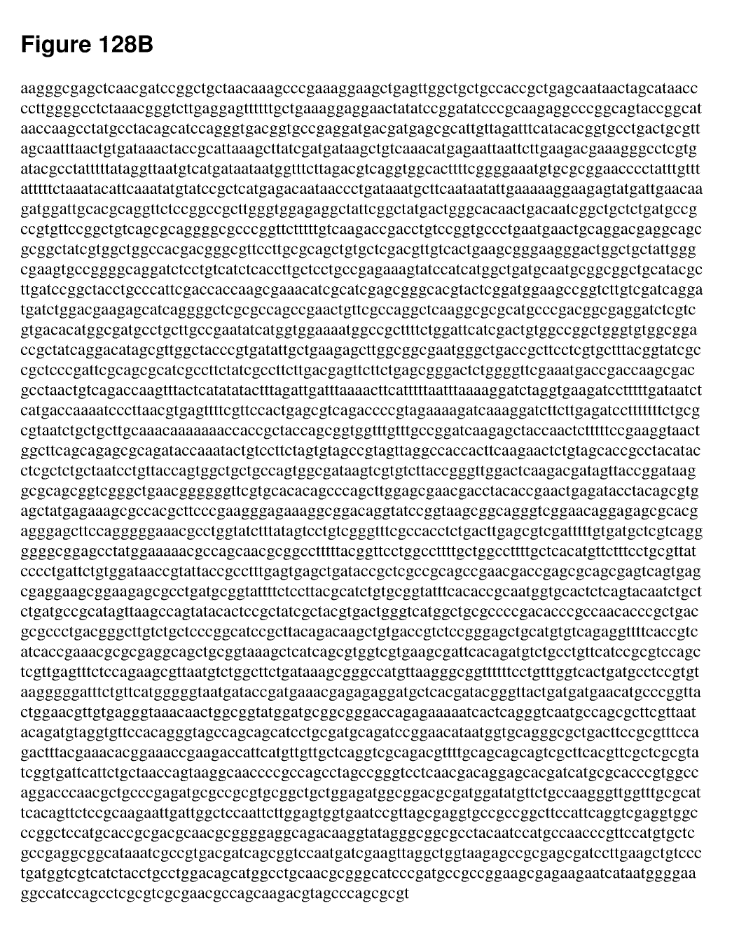

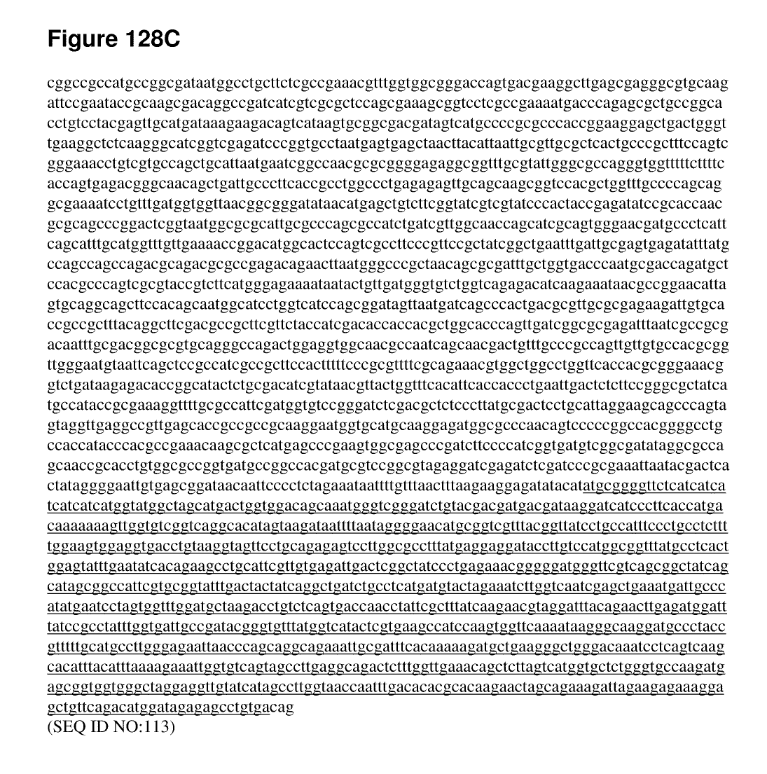

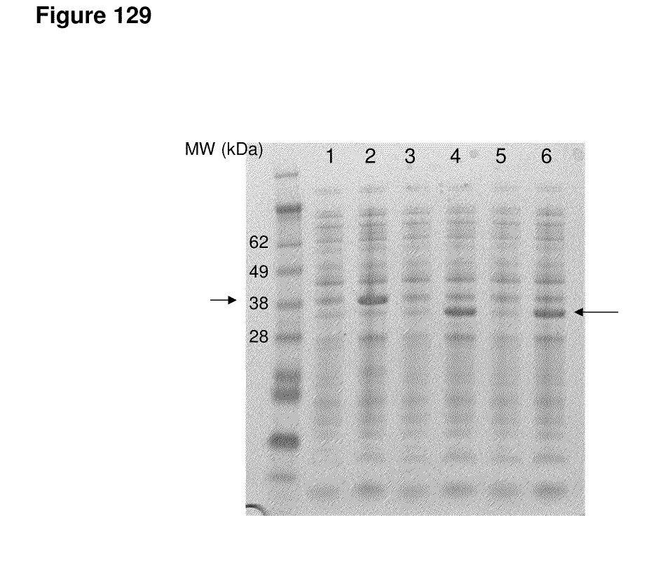

The mvk genes from both Lactobacillus sakei (Danisco strain L110) and Streptococcus pneumoniae R6 (ATCC #BAA-255D-5) were PCR amplified (Table 10 for primer pairs) from genomic DNA, TOPO-cloned into the pET200D-TOPO (Invitrogen) expression vector, and transformed into chemically competent E. coli TOP10 (Invitrogen) cells according to the manufacturer's recommended protocol. Inserts of mvk into pET200D-TOPO, which generates a translational fusion between a 6×His tag and the gene of interest, were verified by PCR using the T7 Forward primer (Table 10) and either of the reverse primers (Lsmvk2 or Spmvk2), respectively. Positive plasmids, which confer kanamycin resistance to E. coli, were purified via miniprep (Qiagen), and the complete mvk insertions were sequenced (Quintara Biosciences) using T7 Forward and T7 Reverse primers (Table 10). The complete sequences for pDW01 (harboring the Lb. sakei mvk gene) and pDW02 (harboring the S. pneumoniae mvk gene) are listed in FIGS. 127B, 127C, 128B, and 128C, respectively. FIGS. 127A and 128A show plasmid maps. The DNA sequence of mvk from Lb. sakei Danisco strain L110 diverged from the sequence of mvk from Lb. sakei strain 23K (NCBI accession #CR936503). The mvk from L110 shared only 92% DNA identity with the mvk of strain 23K, and only 97% amino acid identity. pDW01 and pDW02 were transformed into chemically competent E. coli BL21 Star (DE3) (Invitrogen) cells for expression analysis. Individual strains containing pDW01 and pDW02 were grown at 37° C. overnight in LB medium. The following day, strains were diluted to an OD₆₀₀ of 0.05 and grown at 37° C. to an OD₆₀₀ of approximately 1.0. Cultures were split (to generate both uninduced and induced samples) and IPTG was added to one member of each pair at a concentration of 1 mM. Strains were returned to the incubator and grown for another 2 hours at 37° C. Samples of each culture (approximately 10 μl) were removed for SDS-PAGE analysis using the NuPage system (Invitrogen) according to manufacturer's instructions. FIG. 129 shows that after induction, proteins of approximately 37.8 kDa (for Lb. sakei mvk with the N-terminal 6× His tag, lane 2) and 35.6 kDa (for S. pneumoniae mvk with the N-terminal 6× His tag, lanes 4 and 6) were produced, in comparison to the uninduced control.

TABLE 10
Oligonucleotides

| Primer | | |
|---|---|---|
| Name | Sequence (5′ to 3′) | Purpose |
| Lsmvk1 | CACCATGCAAACGAGTGTGGGAAACAGTCA | Forward primer for |
| | CGCT (SEQ ID NO: 133) | Lb. sakei mvk |
| Lsmvk2 | TGTTTAATTAGTGTGTAGTGCGTGTAATGG | Reverse primer for |
| | (SEQ ID NO: 134) | Lb. sakei mvk |
| Spmvk1 | CACCATGACAAAAAAAGTTGGTGTCGGTCA | Forward primer for |
| | GGCAC (SEQ ID NO: 135) | S. pneumoniae mvk |
| Spmvk2 | CTGTCACAGGCTCTCTATCCATGTCTGAAC | Reverse primer for |
| | (SEQ ID NO: 136) | S. pneumoniae mvk |
| T7 Forward | TAATACGACTCACTATAGGG (SEQ ID | PCR and |
| | NO: 137) | sequencing primer |
| T7 Reverse | GCTAGTTATTGCTCAGCGG (SEQ ID NO: 138) | PCR and |
| | | sequencing primer |

### Example 13

### Production of Isoprene in E. coli Expressing Recombinant Kudzu Isoprene Synthase

I. Construction of Vectors for Expression of the Kudzu Isoprene Synthase in E. coli

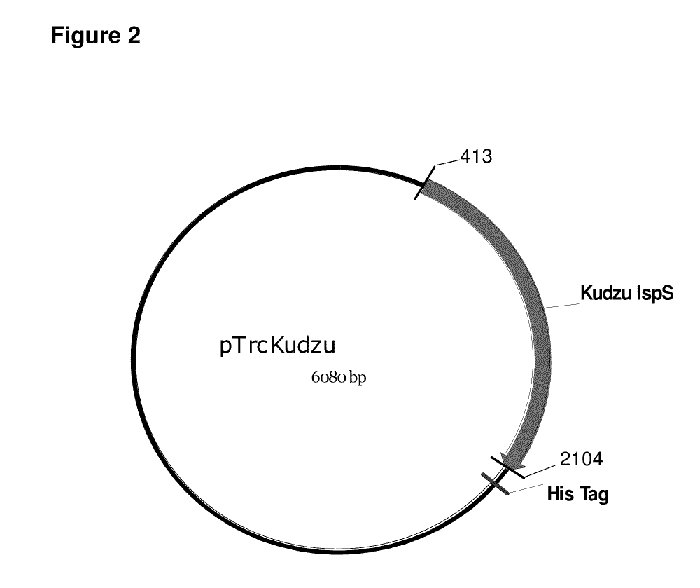

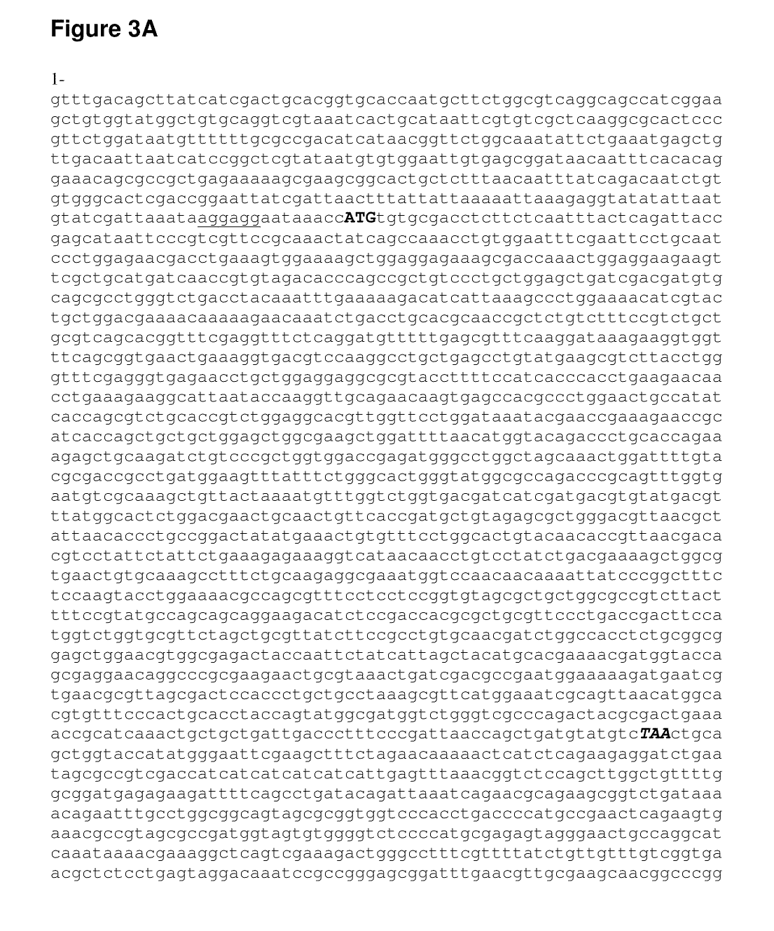

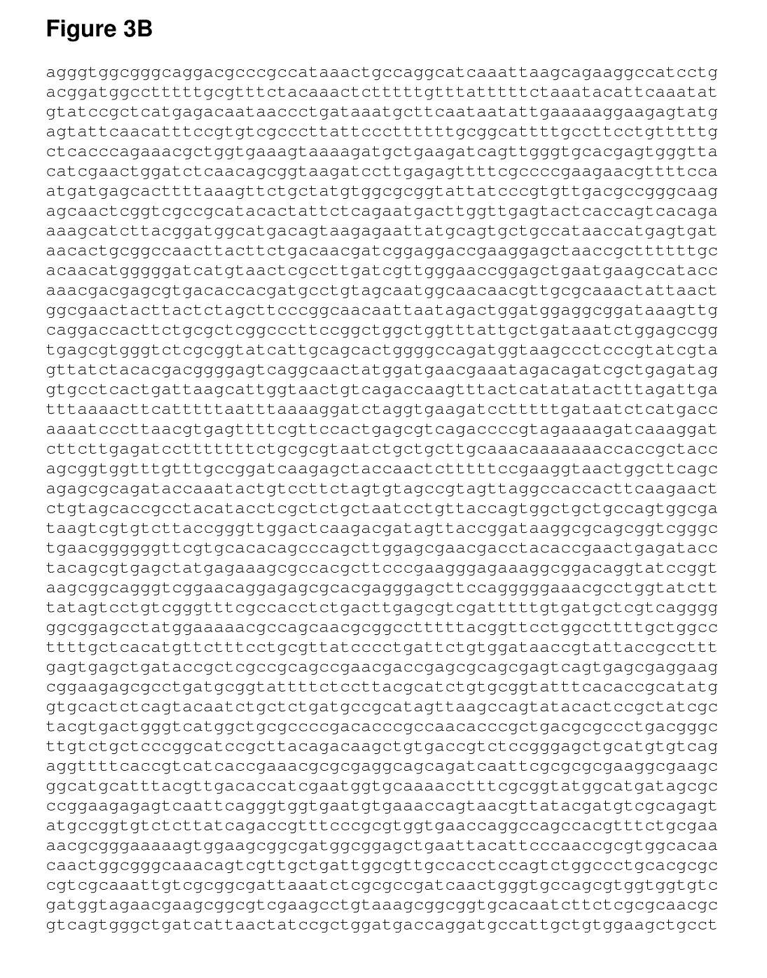

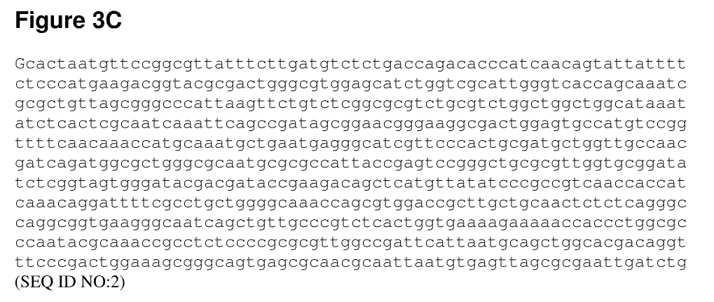

The protein sequence for the kudzu (Pueraria montana) isoprene synthase gene (IspS) was obtained from GenBank (AAQ84170). A kudzu isoprene synthase gene, optimized for E. coli codon usage, was purchased from DNA2.0 (SEQ ID NO:1). The isoprene synthase gene was removed from the supplied plasmid by restriction endonuclease digestion with BspLU11I/PstI, gel-purified, and ligated into pTrcHis2B (Invitrogen) that had been digested with NcoI/PstI. The construct was designed such that the stop codon in the isoprene synthase gene 5′ to the PstI site. As a result, when the construct was expressed the His-Tag is not attached to the isoprene synthase protein. The resulting plasmid, pTrcKudzu, was verified by sequencing (FIGS. 2 and 3).

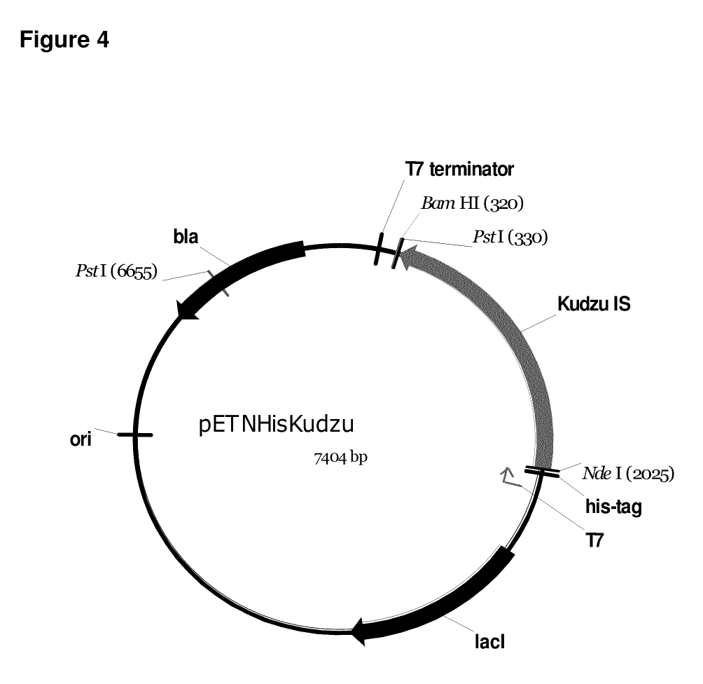

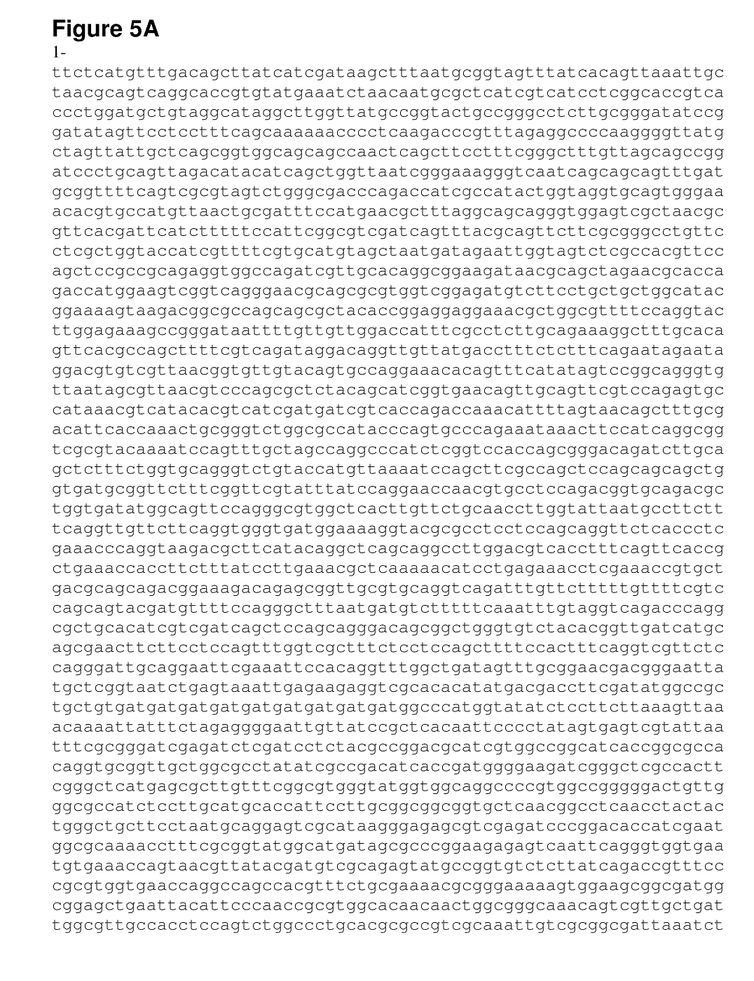

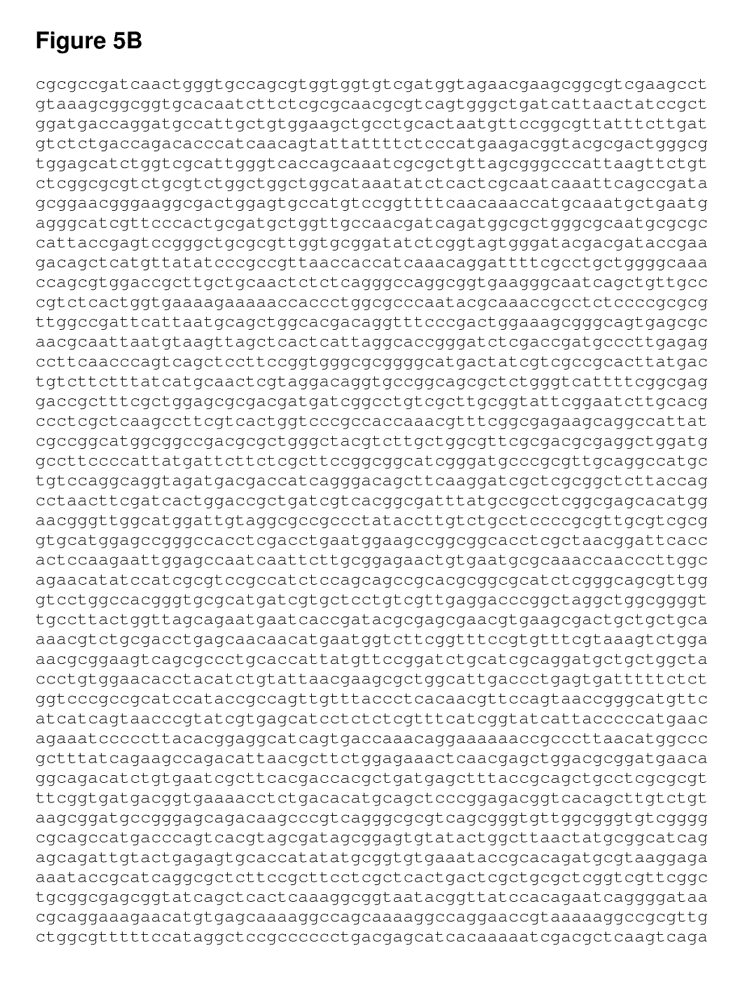

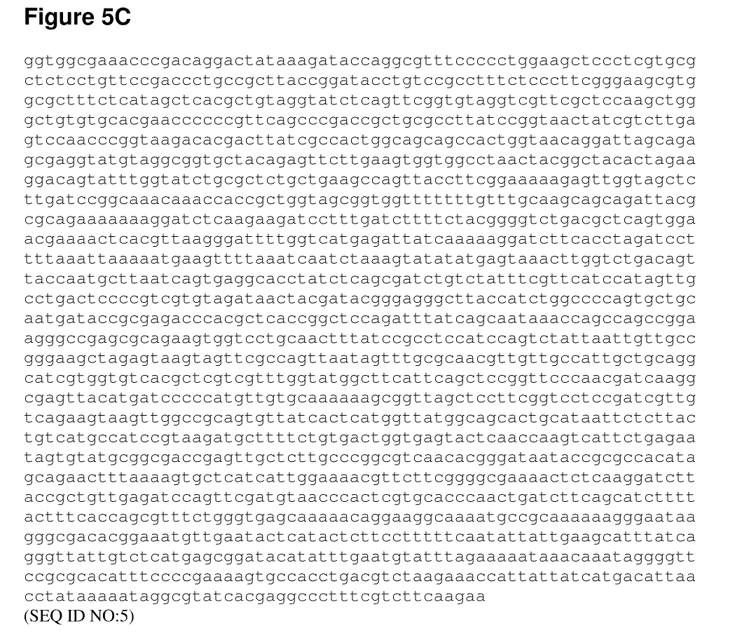

The isoprene synthase gene was also cloned into pET16b (Novagen). In this case, the isoprene synthase gene was inserted into pET16b such that the recombinant isoprene synthase protein contained the N-terminal His tag. The isoprene synthase gene was amplified from pTrcKudzu by PCR using the primer set pET-His-Kudzu-2F: 5′-CGTGAGATCATATGTGTGCGACCTCTTCTCAATTTAC (SEQ ID NO:3) and pET-His-Kudzu-R: 5′-CGGTCGACGGATCCCTGCAGTTAGACATACATCAGCTG (SEQ ID NO:4). These primers added an NdeI site at the 5′-end and a BamH1 site at the 3′ end of the gene respectively. The plasmid pTrcKudzu, described above, was used as template DNA, Herculase polymerase (Stratagene) was used according to manufacture's directions, and primers were added at a concentration of 10 pMols. The PCR was carried out in a total volume of 25 ul. The PCR product was digested with NdeI/BamH1 and cloned into pET16b digested with the same enzymes. The ligation mix was transformed into E. coli Top10 (Invitrogen) and the correct clone selected by sequencing. The resulting plasmid, in which the kudzu isoprene synthase gene was expressed from the T7 promoter, was designated pETNHisKudzu (FIGS. 4 and 5).

The kudzu isoprene synthase gene was also cloned into the low copy number plasmid pCL1920. Primers were used to amplify the kudzu isoprene synthase gene from pTrcKudzu described above. The forward primer added a HindlII site and an E. coli consensus RBS to the 5′ end. The PstI cloning site was already present in pTrcKudzu just 3′ of the stop codon so the reverse primer was constructed such that the final PCR product includes the PstI site. The sequences of the primers were: HindlII-rbs-Kudzu F: 540 -CATATGAAAGCTTGTATCGATTAAATAAGGAGGAATAAACC (SEQ ID NO:6) and BamH1-Kudzu R:

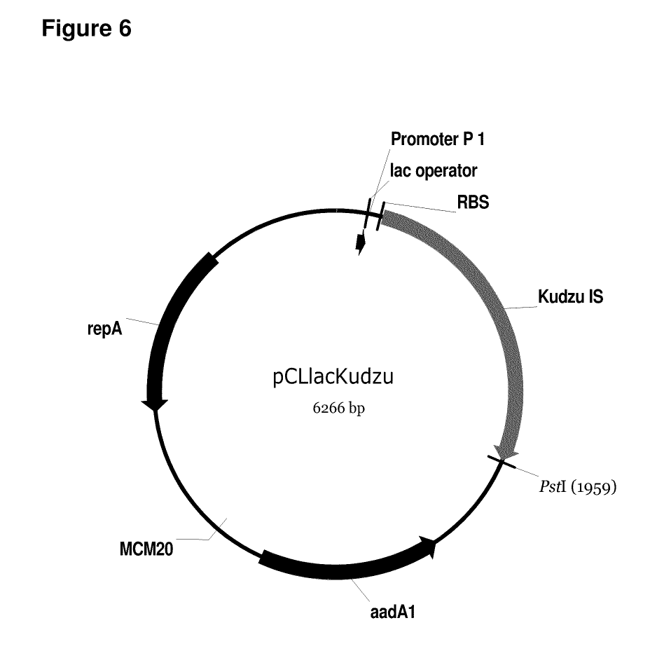

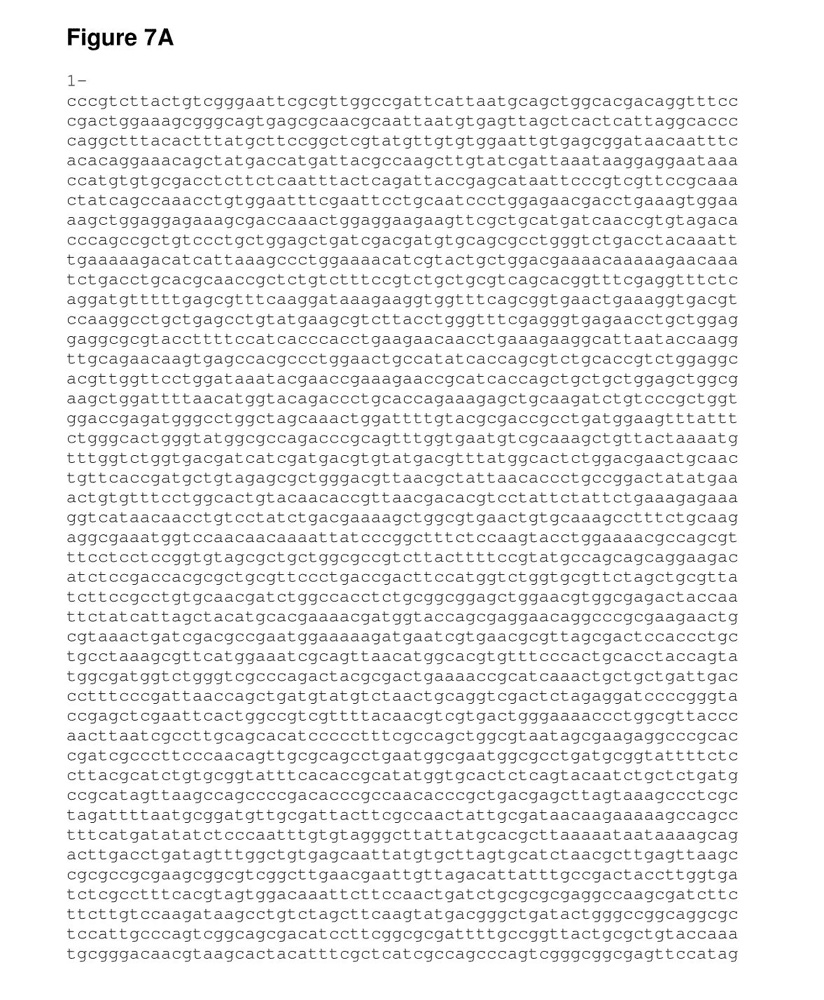

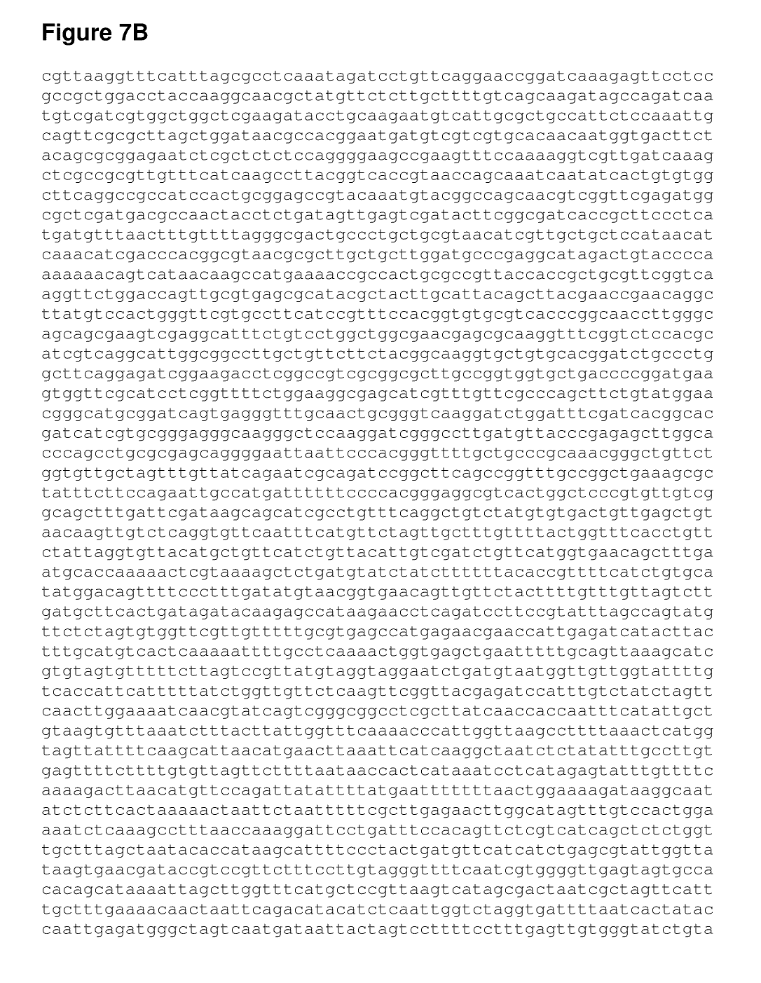

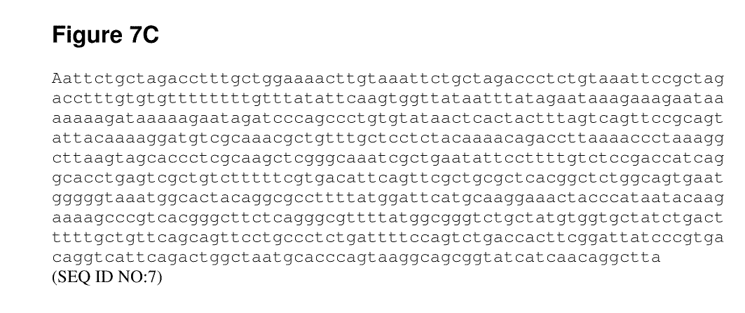

5′-CGGTCGACGGATCCCTGCAGTTAGACATACATCAGCTG (SEQ ID NO:4). The PCR product was amplified using Herculase polymerase with primers at a concentration of 10 pmol and with 1 ng of template DNA (pTrcKudzu). The amplification protocol included 30 cycles of (95° C. for 1 minute, 60° C. for 1 minute, 72° C. for 2 minutes). The product was digested with HindlII and PstI and ligated into pCL1920 which had also been digested with HindlII and PstI. The ligation mix was transformed into E. coli Top10. Several transformants were checked by sequencing. The resulting plasmid was designated pCL-lac-Kudzu (FIGS. 6 and 7A-7C).

### II. Determination of Isoprene Production

For the shake flask cultures, one ml of a culture was transferred from shake flasks to 20 ml CTC headspace vials (Agilent vial cat #5188 2753; cap cat #5188 2759). The cap was screwed on tightly and the vials incubated at the equivalent temperature with shaking at 250 rpm. After 30 minutes the vials were removed from the incubator and analyzed as described below (see Table 1 for some experimental values from this assay).

In cases where isoprene production in fermentors was determined, samples were taken from the off-gas of the fermentor and analyzed directly as described below (see Table 2 for some experimental values from this assay).

The analysis was performed using an Agilent 6890 GC/MS system interfaced with a CTC Analytics (Switzerland) CombiPAL autosampler operating in headspace mode. An Agilent HP-5MS GC/MS column (30 m×0.25 mm; 0.25 μm film thickness) was used for separation of analytes. The sampler was set up to inject 500 μL of headspace gas. The GC/MS method utilized helium as the carrier gas at a flow of 1 ml/min. The injection port was held at 250° C. with a split ratio of 50:1. The oven temperature was held at 37° C. for the 2 minute duration of the analysis. The Agilent 5793N mass selective detector was run in single ion monitoring (SIM) mode on m/z 67. The detector was switched off from 1.4 to 1.7 minutes to allow the elution of permanent gases. Under these conditions isoprene(2-methyl-1,3-butadiene) was observed to elute at 1.78 minutes. A calibration table was used to quantify the absolute amount of isoprene and was found to be linear from 1 μg/L to 2000 μg/L. The limit of detection was estimated to be 50 to 100 ng/L using this method.

III. Production of Isoprene in Shake Flasks Containing E. coli Cells Expressing Recombinant Isoprene Synthase

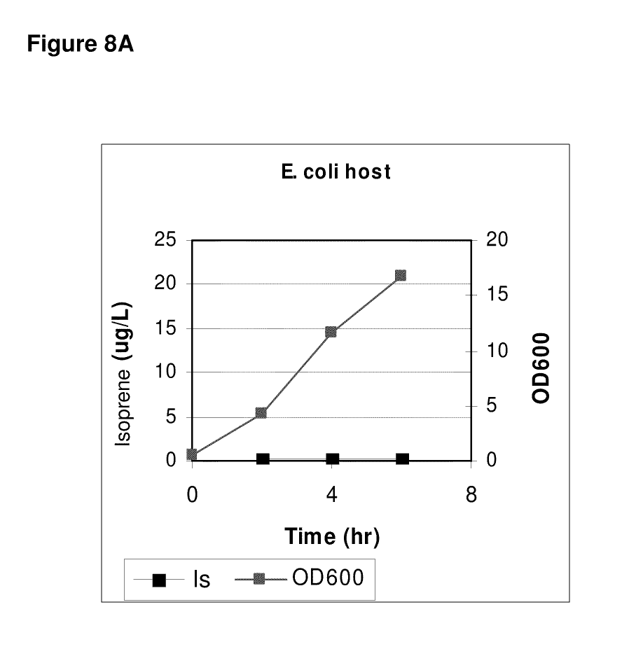

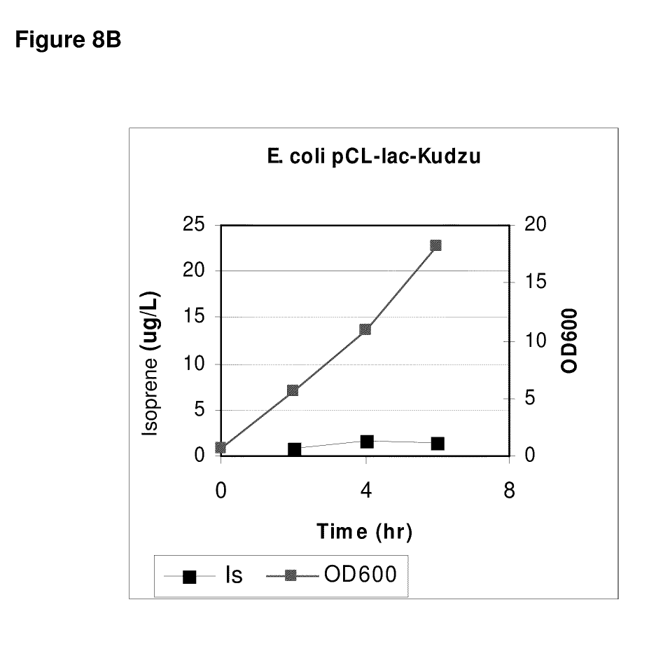

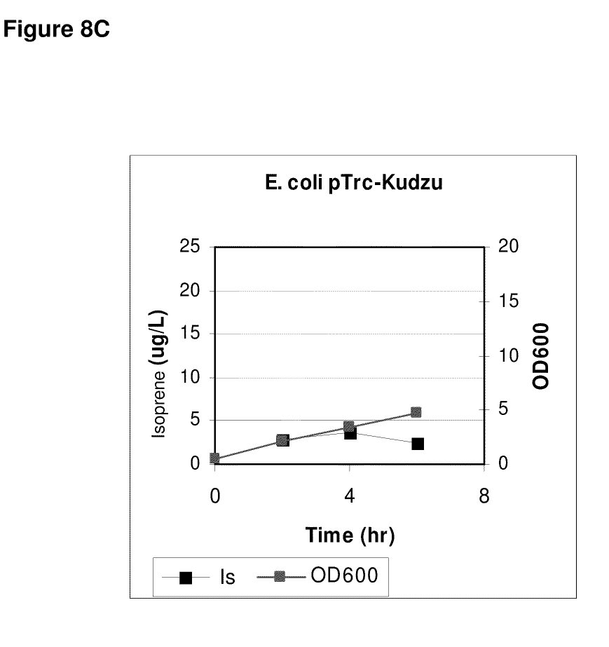

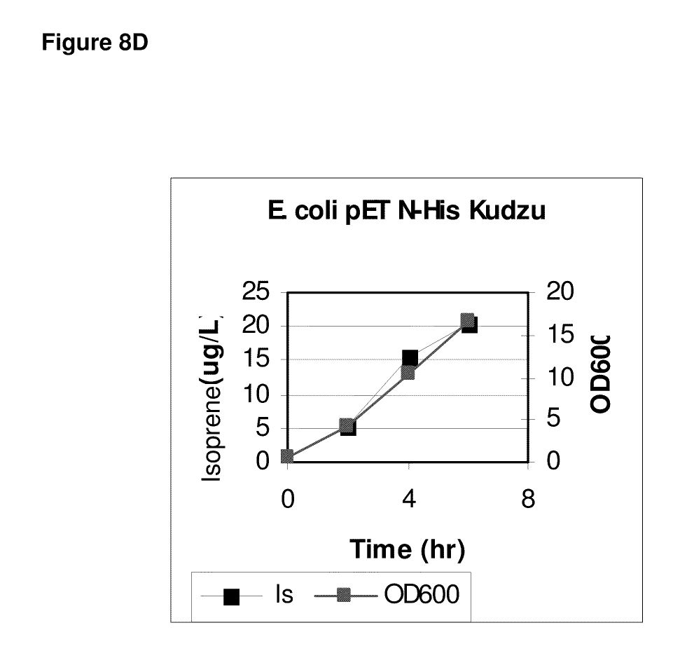

The vectors described above were introduced to E. coli strain BL21 (Novagen) to produce strains BL21/ptrcKudzu, BL21/pCL-lac-Kudzu and BL21/pETHisKudzu. The strains were spread for isolation onto LA (Luria agar)+carbenicillin (50 μg/ml) and incubated overnight at 37° C. Single colonies were inoculated into 250 ml baffled shake flasks containing 20 ml Luria Bertani broth (LB) and carbenicillin (100 μg/ml). Cultures were grown overnight at 20° C. with shaking at 200 rpm. The OD₆₀₀ of the overnight cultures were measured and the cultures were diluted into a 250 ml baffled shake flask containing 30 ml MagicMedia (Invitrogen)+carbenicillin (100 μg/ml) to an OD₆₀₀˜0.05. The culture was incubated at 30° C. with shaking at 200 rpm. When the OD₆₀₀˜0.5-0.8, 400 μM IPTG was added and the cells were incubated for a further 6 hours at 30° C. with shaking at 200 rpm. At 0, 2, 4 and 6 hours after induction with IPTG, 1 ml aliquots of the cultures were collected, the OD₆₀₀ was determined and the amount of isoprene produced was measured as described above. Results are shown in FIGS. 8A-8D.

IV. Production of Isoprene from BL21/ptrcKudzu in 14 Liter Fermentation

Large scale production of isoprene from E. coli containing the recombinant kudzu isoprene synthase gene was determined from a fed-batch culture. The recipe for the fermentation media (TM2) per liter of fermentation medium was as follows: K₂HPO₄ 13.6 g, KH₂PO₄ 13.6 g, MgSO₄* 7H₂O 2 g, citric acid monohydrate 2 g, ferric ammonium citrate 0.3 g, (NH₄)₂SO₄ 3.2 g, yeast extract 5 g, 1000× Modified Trace Metal Solution 1 ml. All of the components were added together and dissolved in diH₂O. The pH was adjusted to 6.8 with potassium hydroxide (KOH) and q.s. to volume. The final product was filter sterilized with 0.22μ filter (only, do not autoclave). The recipe for 1000× Modified Trace Metal Solution was as follows: Citric Acids*H₂O 40 g, MnSO₄*H₂O 30 g, NaCl 10 g, FeSO₄*7H₂O 1 g, CoCl₂*6H₂O 1 g, ZnSO₄*7H₂O 1 g, CuSO₄*5H₂O 100 mg, H₃BO₃ 100 mg, NaMoO₄*2H₂O 100 mg. Each component was dissolved one at a time in diH₂O, pH to 3.0 with HCl/NaOH, then q.s. to volume and filter sterilized with a 0.22μ filter.

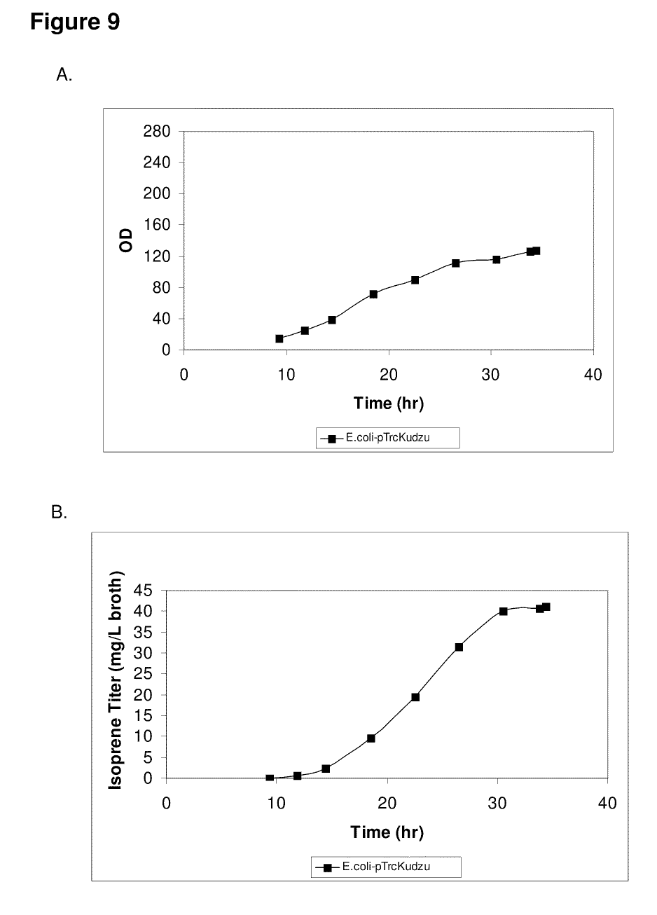

This experiment was carried out in 14 L bioreactor to monitor isoprene formation from glucose at the desired fermentation, pH 6.7 and temperature 34° C. An inoculum of E. coli strain BL21/ptrcKudzu taken from a frozen vial was prepared in soytone-yeast extract-glucose medium. After the inoculum grew to OD₅₅₀=0.6, two 600 ml flasks were centrifuged and the contents resuspended in 70 ml supernatant to transfer the cell pellet (70 ml of OD 3.1 material) to the bioreactor. At various times after inoculation, samples were removed and the amount of isoprene produced was determined as described above. Results are shown in FIGS. 9A and 9B.

### Example 14

### Production of Isoprene in E. coli Expressing Recombinant Poplar Isoprene Synthase

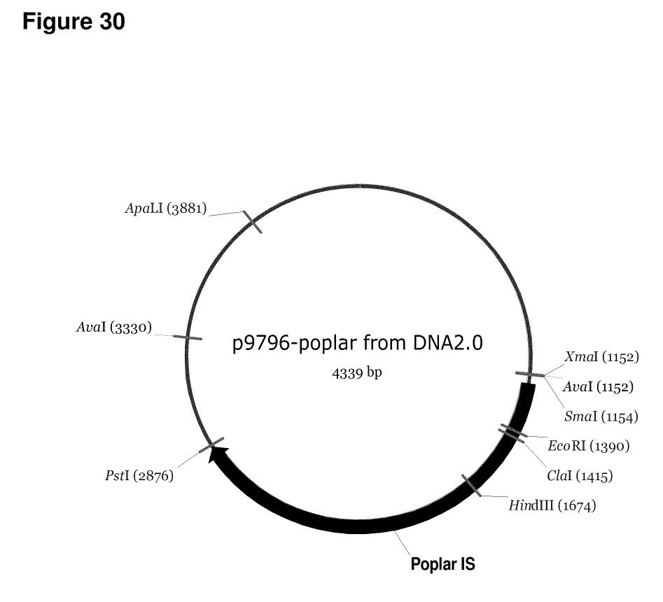

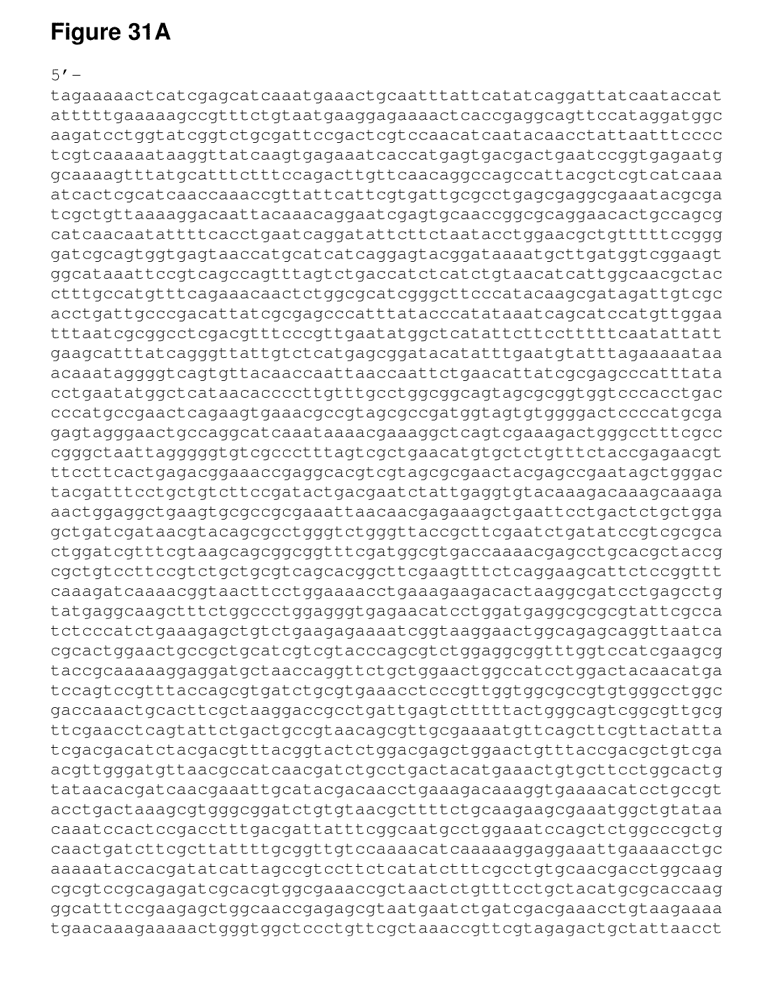

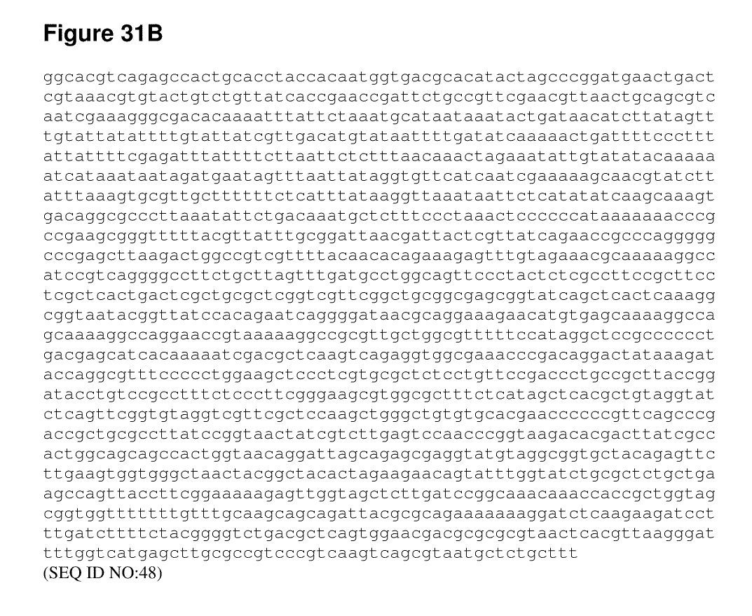

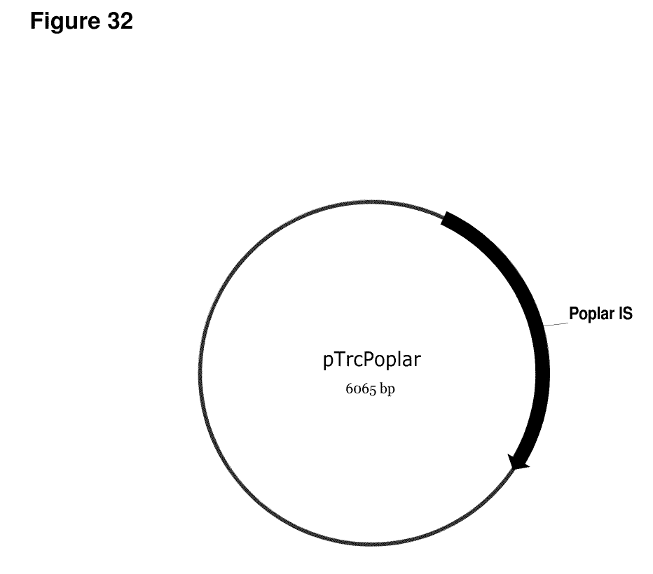

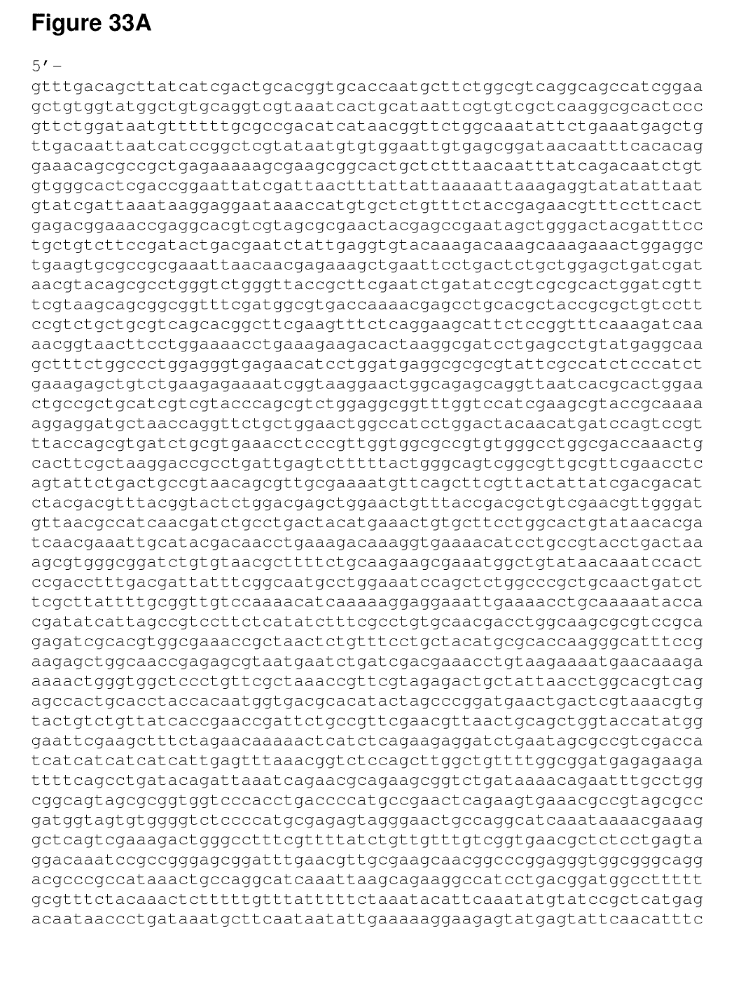

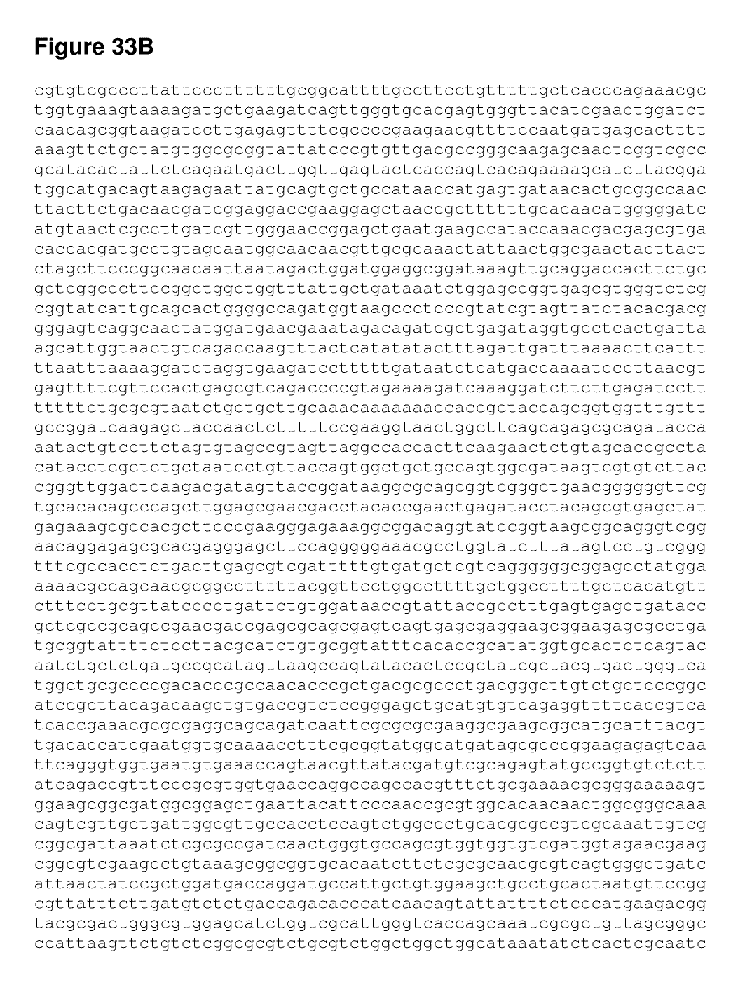

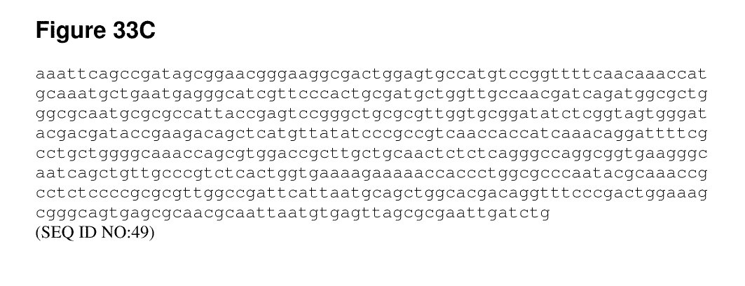

The protein sequence for the poplar (Populus alba×Populus tremula) isoprene synthase (Schnitzler, J-P, et al. (2005) Planta 222:777-786) was obtained from GenBank (CAC35696). A gene, codon optimized for E. coli, was purchased from DNA2.0 (p9796-poplar, FIGS. 30 and 31A and 31B). The isoprene synthase gene was removed from the supplied plasmid by restriction endonuclease digestion with BspLU11I/PstI, gel-purified, and ligated into pTrcHis2B that had been digested with NcoI/PstI. The construct is cloned such that the stop codon in the insert is before the PstI site, which results in a construct in which the His-Tag is not attached to the isoprene synthase protein. The resulting plasmid pTrcPoplar (FIGS. 32 and 33A-33C), was verified by sequencing.

### Example 15

### Production of Isoprene in Panteoa citrea Expressing Recombinant Kudzu Isoprene Synthase

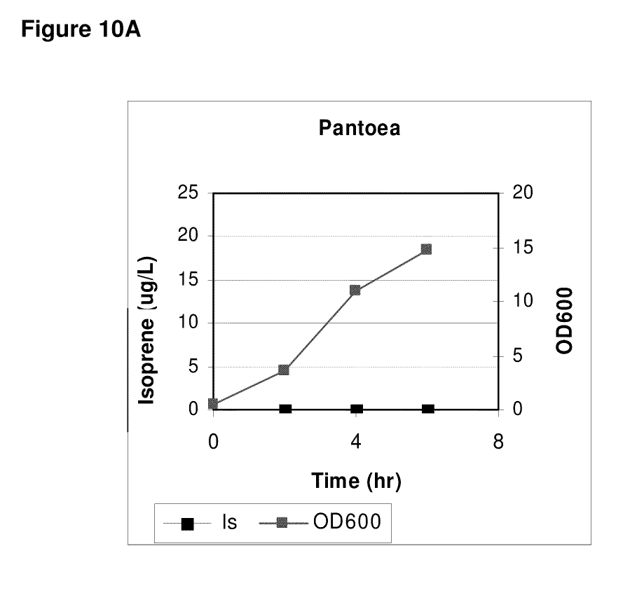

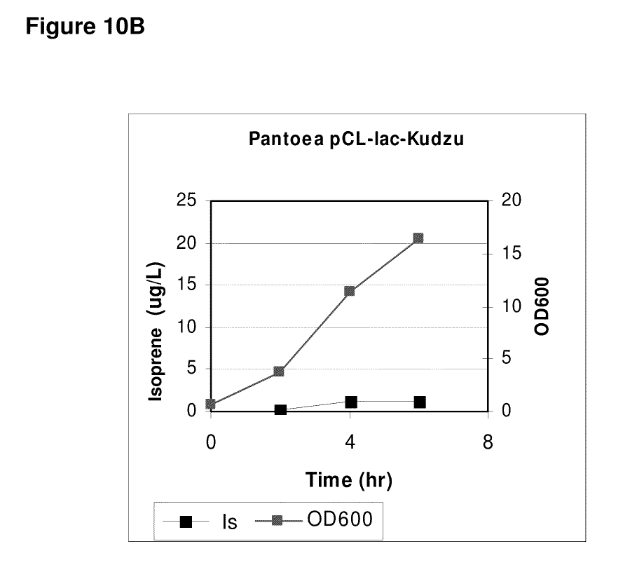

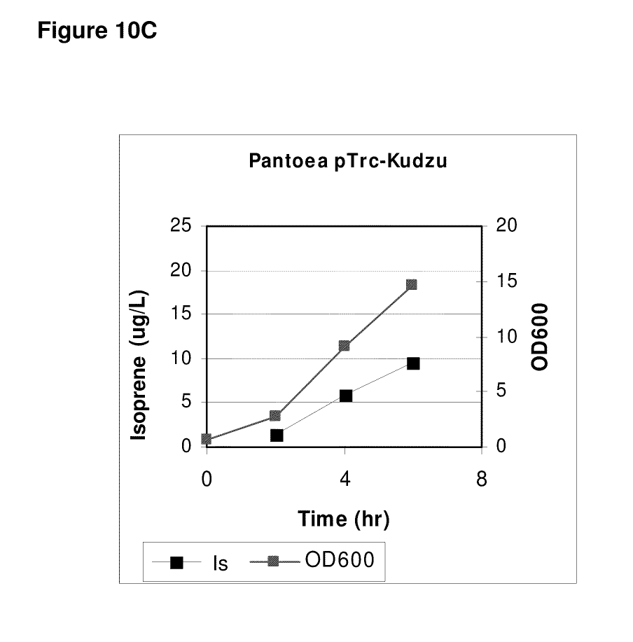

The pTrcKudzu and pCL-lac Kudzu plasmids described in Example 13 were electroporated into P. citrea (U.S. Pat. No. 7,241,587). Transformants were selected on LA containing carbenicillin (200 μg/ml) or spectinomycin (50 μg/ml) respectively. Production of isoprene from shake flasks and determination of the amount of isoprene produced was performed as described in Example 13 for E. coli strains expressing recombinant kudzu isoprene synthase. Results are shown in FIGS. 10A-10C.

### Example 16

### Production of Isoprene in Bacillus subtilis Expressing Recombinant Kudzu Isoprene Synthase

I. Construction of a B. subtilis Replicating Plasmid for the Expression of Kudzu Isoprene Synthase

The kudzu isoprene synthase gene was expressed in Bacillus subtilis aprEnprE PxyI-comK strain (BG3594comK) using a replicating plasmid (pBS19 with a chloramphenicol resistance cassette) under control of the aprE promoter. The isoprene synthase gene, the aprE promoter and the transcription terminator were amplified separately and fused using PCR. The construct was then cloned into pBS19 and transformed into B. subtilis.

a) Amplification of the aprE Promoter

The aprE promoter was amplified from chromosomal DNA from Bacillus subtilis using the following primers:

CF 797 (+) Start aprE promoter MfeI

| | |
|---|---|
| 5′-GACATCAATTGCTCCATTTTCTTCTGCTATC | (SEQ ID NO: 58) |

CF 07-43 (−) Fuse aprE promoter to Kudzu ispS

(SEQ ID NO: 59)

′-ATTGAGAAGAGGTCGCACACACTCTTTACCCTCTCCTTTTA

### b) Amplification of the Isoprene Synthase Gene

The kudzu isoprene synthase gene was amplified from plasmid pTrcKudzu (SEQ ID NO:2). The gene had been codon optimized for E. coli and synthesized by DNA 2.0. The following primers were used:

CF 07-42 (+) Fuse the aprE promoter to kudzu isoprene synthase gene (GTG start codon)

(SEQ ID NO: 60)

5′-TAAAAGGAGAGGGTAAAGAGTGTGTGCGACCTCTTCTCAAT

CF 07-45 (−) Fuse the 3′ end of kudzu isoprene synthase gene to the terminator

(SEQ ID NO: 61)

5′-CCAAGGCCGGTTTTTTTTAGACATACATCAGCTGGTTAATC

### c) Amplification of the Transcription Terminator

The terminator from the alkaline serine protease of Bacillus amyliquefaciens was amplified from a previously sequenced plasmid pJHPms382 using the following primers:

CF 07-44 (+) Fuse the 3′ end of kudzu isoprene synthase to the terminator

(SEQ ID NO: 62)

5′-GATTAACCAGCTGATGTATGTCTAAAAAAAACCGGCCTTGG

CF 07-46 (−) End of B. amyliquefaciens terminator (BamHI)

(SEQ ID NO: 63)

5′-GACATGACGGATCCGATTACGAATGCCGTCTC

The kudzu fragment was fused to the terminator fragment using PCR with the following primers:

CF 07-42 (+) Fuse the aprE promoter to kudzu isoprene synthase gene (GTG start codon)

(SEQ ID NO: 61)

5′-TAAAAGGAGAGGGTAAAGAGTGTGTGCGACCTCTTCTCAAT

CF 07-46 (−) End of B. amyliquefaciens terminator (BamHI)

(SEQ ID NO: 63)

5′-GACATGACGGATCCGATTACGAATGCCGTCTC

The kudzu-terminator fragment was fused to the promoter fragment using PCR with the following primers:

CF 797 (+) Start aprE promoter MfeI

(SEQ ID NO: 64)

5′-GACATCAATTGCTCCATTTTCTTCTGCTATC

CF 07-46 (−) End of B. amyliquefaciens terminator (BamHI)

(SEQ ID NO: 63)
5′-GACATGACGGATCCGATTACGAATGCCGTCTC

The fusion PCR fragment was purified using a Qiagen kit and digested with the restriction enzymes MfeI and BamHI. This digested DNA fragment was gel purified using a Qiagen kit and ligated to a vector known as pBS19, which had been digested with EcoRI and BamHI and gel purified.

The ligation mix was transformed into E. coli Top 10 cells and colonies were selected on LA+50 carbenicillin plates. A total of six colonies were chosen and grown overnight in LB+50 carbenicillin and then plasmids were isolated using a Qiagen kit. The plasmids were digested with EcoRI and BamHI to check for inserts and three of the correct plasmids were sent in for sequencing with the following primers:

CF 149 (+) EcoRI start of aprE promoter

| | |
|---|---|
| 5′-GACATGAATTCCTCCATTTTCTTCTGC | (SEQ ID NO: 65) |

CF 847 (+) Sequence in pXX 049 (end of aprE promoter)

| | |
|---|---|
| 5′-AGGAGAGGGTAAAGAGTGAG | (SEQ ID NO: 66) |

CF 07-45 (−) Fuse the 3′ end of kudzu isoprene synthase to the terminator

(SEQ ID NO: 61)
5′-CCAAGGCCGGTTTTTTTTAGACATACATCAGCTGGTTAATC

CF 07-48 (+) Sequencing primer for kudzu isoprene synthase

| | |
|---|---|
| 5′-CTTTTCCATCACCCACCTGAAG | (SEQ ID NO: 67) |

CF 07-49 (+) Sequencing in kudzu isoprene synthase

| | |
|---|---|
| 5′-GGCGAAATGGTCCAACAACAAAATTATC | (SEQ ID NO: 68) |

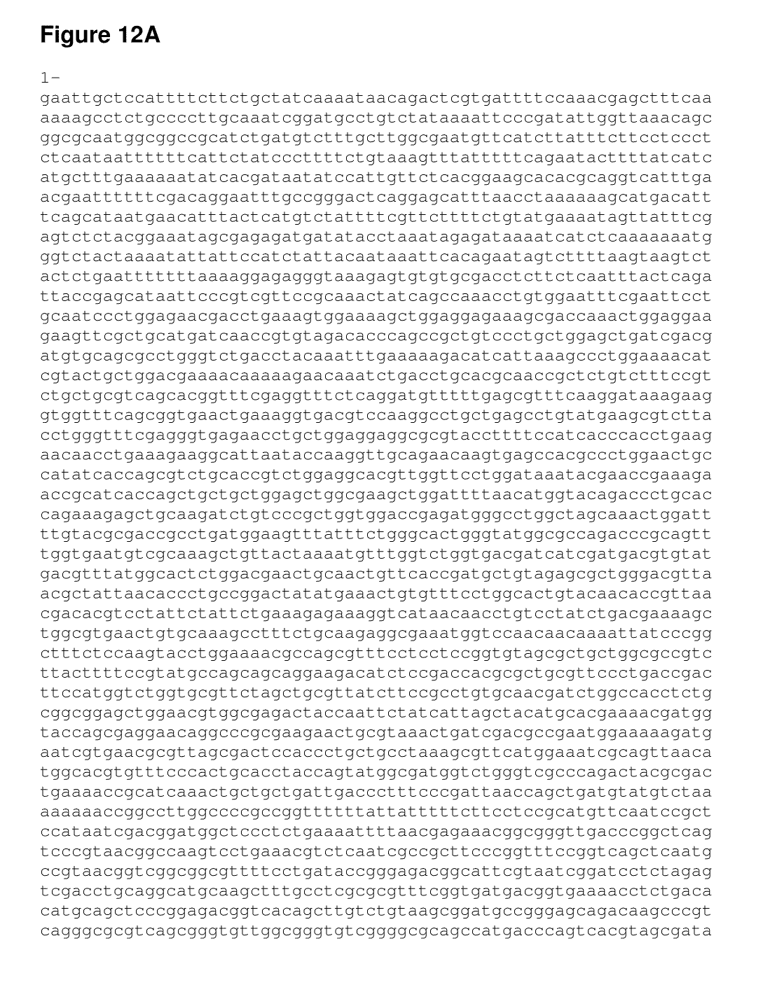

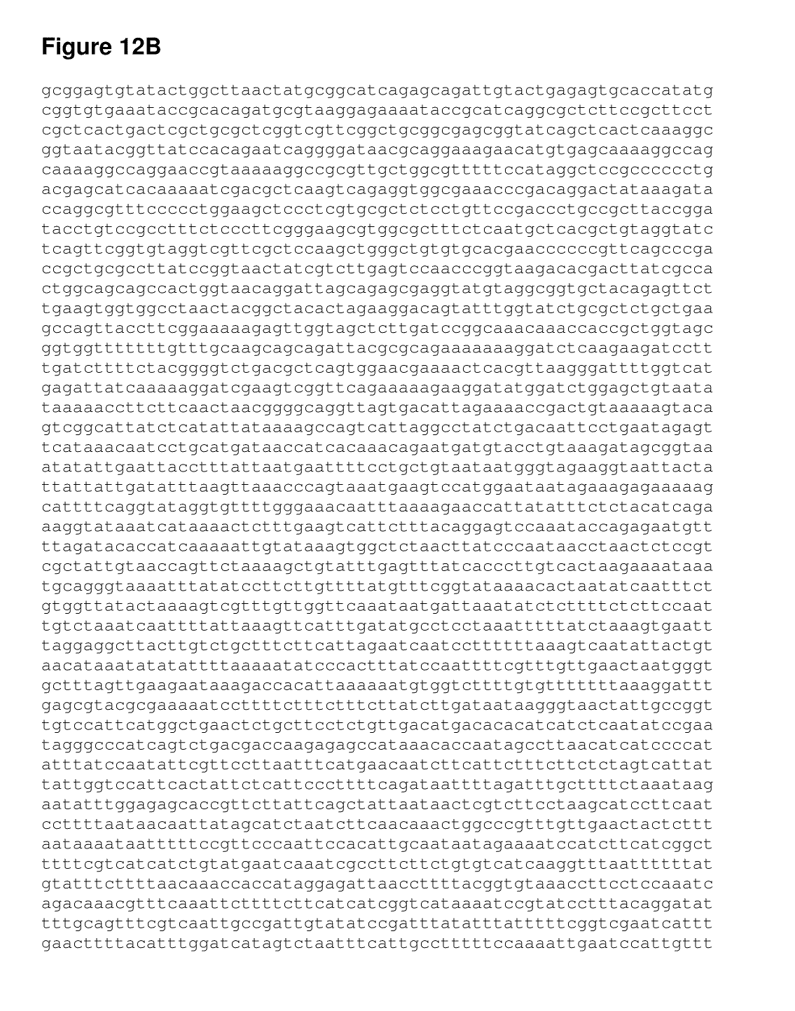

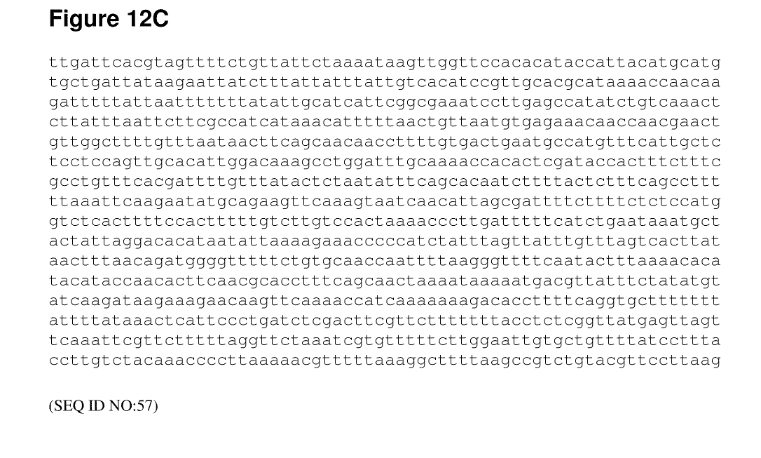

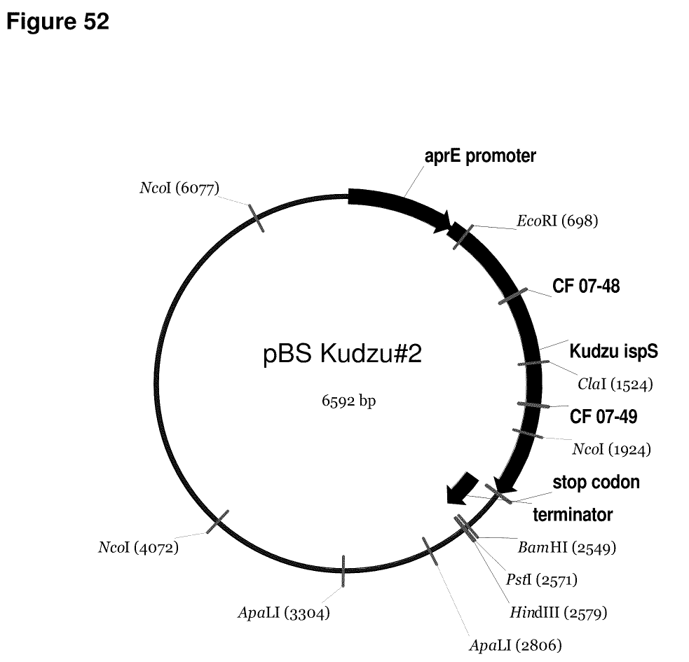

The plasmid designated pBS Kudzu #2 (FIGS. 52 and 12A-12C) was correct by sequencing and was transformed into BG 3594 comK, a Bacillus subtilis host strain. Selection was done on LA+5 chloramphenicol plates. A transformant was chosen and struck to single colonies on LA+5 chloramphenicol, then grown in LB+5 chloramphenicol until it reached an OD₆₀₀ of 1.5. It was stored frozen in a vial at −80° C. in the presence of glycerol. The resulting strain was designated CF 443.

II. Production of Isoprene in Shake Flasks Containing B. subtilis Cells Expressing Recombinant Isoprene Synthase

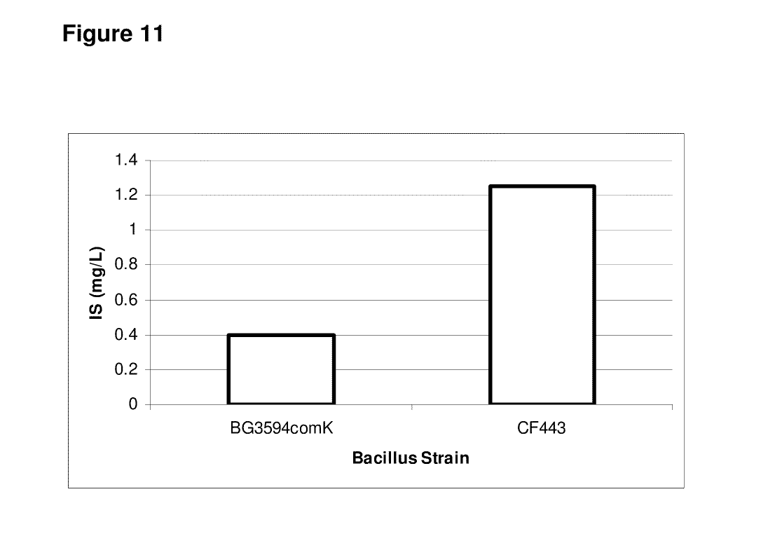

Overnight cultures were inoculated with a single colony of CF 443 from a LA+Chloramphenicol (Cm, 25 μg/ml). Cultures were grown in LB+Cm at 37° C. with shaking at 200 rpm. These overnight cultures (1 ml) were used to inoculate 250 ml baffled shake flasks containing 25 ml Grants II media and chloramphenicol at a final concentration of 25 μg/ml. Grants II Media recipe was 10 g soytone, 3 ml 1M K₂HPO₄, 75 g glucose, 3.6 g urea, 100 ml 10× MOPS, q.s. to 1 L with H₂O, pH 7.2; 10× MOPS recipe was 83.72 g MOPS, 7.17 g tricine, 12 g KOH pellets, 10 ml 0.276M K₂SO₄ solution, 10 ml 0.528 M MgCl₂ solution, 29.22 g NaCl, 100 ml 100× micronutrients, q.s. to 1 L with H₂O; and 100× micronutrients recipe was 1.47 g CaCl₂*2H₂O, 0.4 g FeSO₄*7H₂O, 0.1 g MnSO₄*H₂O, 0.1 g ZnSO₄*H₂O, 0.05 g CuCl₂*2H₂O, 0.1 g CoCl₂*6H₂O, 0.1 g Na₂MoO₄*2H₂O, q.s. to 1 L with H₂O. Shake flasks were incubated at 37° C. and samples were taken at 18, 24, and 44 hours. At 18 hours the headspaces of CF443 and the control strain were sampled. This represented 18 hours of accumulation of isoprene. The amount of isoprene was determined by gas chromatography as described in Example 13. Production of isoprene was enhanced significantly by expressing recombinant isoprene synthase (FIG. 11).

### III. Production of Isoprene by CF443 in 14 L Fermentation

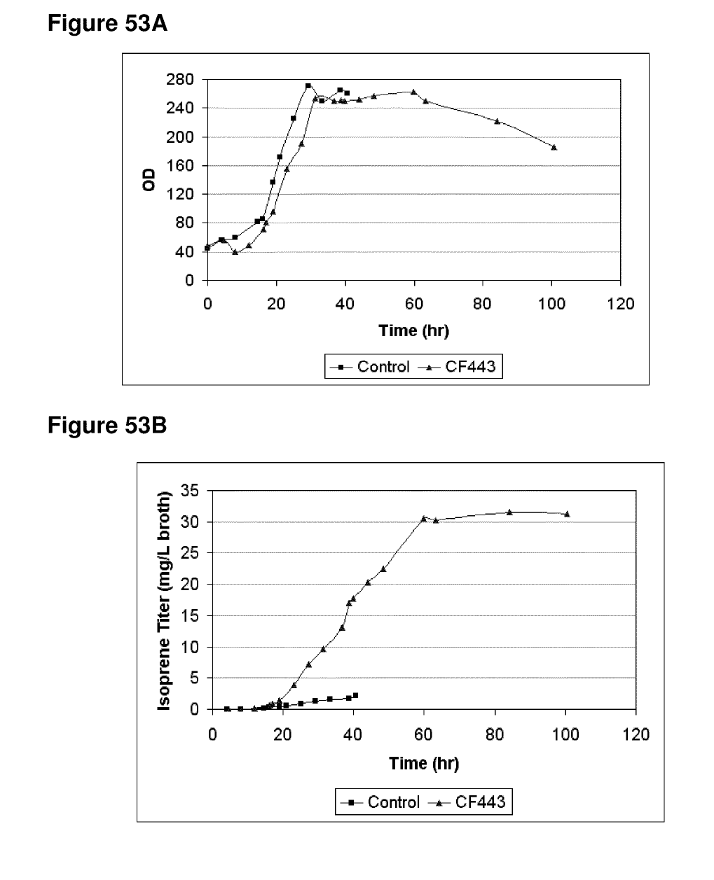

Large scale production of isoprene from B. subtilis containing the recombinant kudzu isoprene synthase gene on a replication plasmid was determined from a fed-batch culture. Bacillus strain CF 443, expressing a kudzu isoprene synthase gene, or control stain which does not express a kudzu isoprene synthase gene were cultivated by conventional fed-batch fermentation in a nutrient medium containing soy meal (Cargill), sodium and potassium phosphate, magnesium sulfate and a solution of citric acid, ferric chloride and manganese chloride. Prior to fermentation the media is macerated for 90 minutes using a mixture of enzymes including cellulases, hemicellulases and pectinases (see, WO95/04134). 14-L batch fermentations are fed with 60% wt/wt glucose (Cargill DE99 dextrose, ADM Versadex greens or Danisco invert sugar) and 99% wt/wt oil (Western Family soy oil, where the 99% wt/wt is the concentration of oil before it was added to the cell culture medium). Feed was started when glucose in the batch was non-detectable. The feed rate was ramped over several hours and was adjusted to add oil on an equal carbon basis. The pH was controlled at 6.8-7.4 using 28% w/v ammonium hydroxide. In case of foaming, antifoam agent was added to the media. The fermentation temperature was controlled at 37° C. and the fermentation culture was agitated at 750 rpm. Various other parameters such as pH, DO %, airflow, and pressure were monitored throughout the entire process. The DO % is maintained above 20. Samples were taken over the time course of 36 hours and analyzed for cell growth (OD₅₅₀) and isoprene production. Results of these experiments are presented in FIGS. 53A and 53B.

IV. Integration of the Kudzu Isoprene Synthase (ispS) in B. subtilis.

The kudzu isoprene synthase gene was cloned in an integrating plasmid (pJH101-cmpR) under the control of the aprE promoter. Under the conditions tested, no isoprene was detected.

### Example 17

### Production of Isoprene in Trichoderma

I. Construction of Vectors for Expression of the Kudzu Isoprene Synthase in Trichoderma reesei

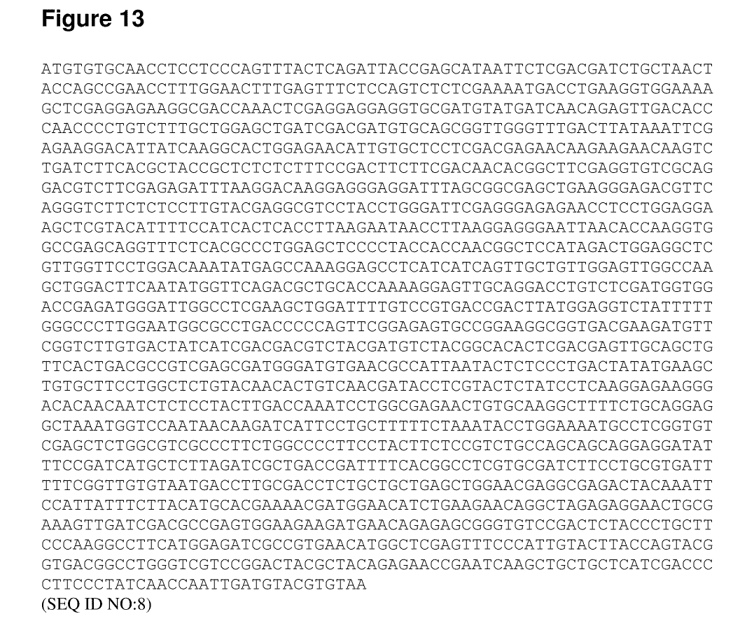

The Yarrowia lipolytica codon-optimized kudzu IS gene was synthesized by DNA 2.0 (SEQ ID NO:8) (FIG. 13). This plasmid served as the template for the following PCR amplification reaction: 1 μl plasmid template (20 ng/ul), 1 μl Primer EL-945 (10 uM) 5′-GCTTATGGATCCTCTAGACTATTACACGTACATCAATTGG (SEQ ID NO:9), 1 μl Primer EL-965 (10 uM) 5′-CACCATGTGTGCAACCTCCTCCCAGTTTAC (SEQ ID NO:10), 1 μl dNTP (10 mM), 5 μl 10× PfuUltra II Fusion HS DNA Polymerase Buffer, 1 μl PfuUltra II Fusion HS DNA Polymerase, 40 μl water in a total reaction volume of 50 μl. The forward primer contained an additional 4 nucleotides at the 5′-end that did not correspond to the Y. lipolytica codon-optimized kudzu isoprene synthase gene, but was required for cloning into the pENTR/D-TOPO vector. The reverse primer contained an additional 21 nucleotides at the 5′-end that did not correspond to the Y. lipolytica codon-optimized kudzu isoprene synthase gene, but were inserted for cloning into other vector backbones. Using the MJ Research PTC-200 Thermocycler, the PCR reaction was performed as follows: 95° C. for 2 minutes (first cycle only), 95° C. for 30 seconds, 55° C. for 30 seconds, 72° C. for 30 seconds (repeat for 27 cycles), 72° C. for 1 minute after the last cycle. The PCR product was analyzed on a 1.2% E-gel to confirm successful amplification of the Y. lipolytica codon-optimized kudzu isoprene synthase gene.

The PCR product was then cloned using the TOPO pENTR/D-TOPO Cloning Kit following manufacturer's protocol: 1 μl PCR reaction, 1 μl Salt solution, 1 μl TOPO pENTR/D-TOPO vector and 3 μl water in a total reaction volume of 6 μl. The reaction was incubated at room temperature for 5 minutes. One microliter of TOPO reaction was transformed into TOP10 chemically competent E. coli cells. The transformants were selected on LA+50 μg/ml kanamycin plates. Several colonies were picked and each was inoculated into a 5 ml tube containing LB+50 μg/ml kanamycin and the cultures grown overnight at 37° C. with shaking at 200 rpm. Plasmids were isolated from the overnight culture tubes using QIAprep Spin Miniprep Kit, following manufacturer's protocol. Several plasmids were sequenced to verify that the DNA sequence was correct.

A single pENTR/D-TOPO plasmid, encoding a Y. lipolytica codon-optimized kudzu isoprene synthase gene, was used for Gateway Cloning into a custom-made pTrex3g vector. Construction of pTrex3g is described in WO 2005/001036 A2. The reaction was performed following manufacturer's protocol for the Gateway LR Clonase II Enzyme Mix Kit (Invitrogen): 1 μl Y. lipolytica codon-optimized kudzu isoprene synthase gene pENTR/D-TOPO donor vector, 1 μl pTrex3g destination vector, 6 μl TE buffer, pH 8.0 in a total reaction volume of 8 μl. The reaction was incubated at room temperature for 1 hour and then 1 μl proteinase K solution was added and the incubation continued at 37° C. for 10 minutes. Then 1 μl of reaction was transformed into TOP10 chemically competent E. coli cells. The transformants were selected on LA+50 μg/ml carbenicillin plates. Several colonies were picked and each was inoculated into a 5 ml tube containing LB+50 μg/ml carbenicillin and the cultures were grown overnight at 37° C. with shaking at 200 rpm. Plasmids were isolated from the overnight culture tubes using QIAprep Spin Miniprep Kit (Qiagen, Inc.), following manufacturer's protocol. Several plasmids were sequenced to verify that the DNA sequence was correct.

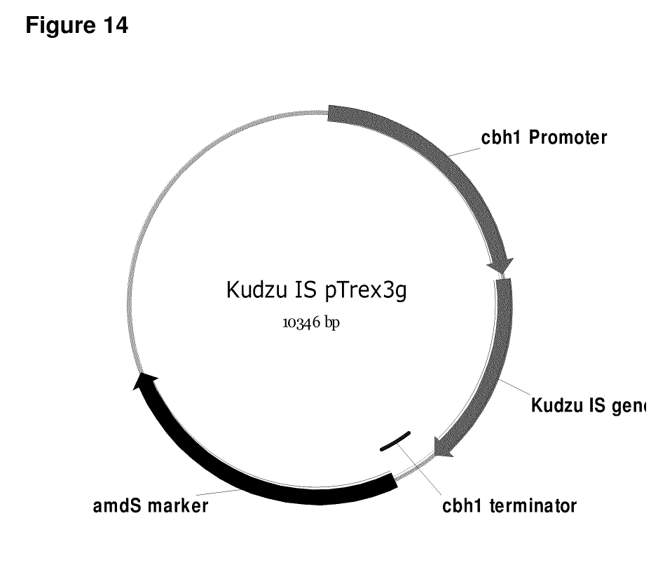

Biolistic transformation of Y. lipolytica codon-optimized kudzu isoprene synthase pTrex3g plasmid (FIG. 14) into a quad delete Trichoderma reesei strain was performed using the Biolistic PDS-1000/HE Particle Delivery System (see WO 2005/001036 A2). Isolation of stable transformants and shake flask evaluation was performed using protocol listed in Example 11 of patent publication WO 2005/001036 A2.

II. Production of Isoprene in Recombinant Strains of T. reesei

One ml of 15 and 36 hour old cultures of isoprene synthase transformants described above were transferred to head space vials. The vials were sealed and incubated for 5 hours at 30° C. Head space gas was measured and isoprene was identified by the method described in Example 13. Two of the transformants showed traces of isoprene. The amount of isoprene could be increased by a 14 hour incubation. The two positive samples showed isoprene at levels of about 0.5 μg/L for the 14 hour incubation. The untransformed control showed no detectable levels of isoprene. This experiment shows that T. reesei is capable of producing isoprene from endogenous precursor when supplied with an exogenous isoprene synthase.

### Example 18

### Production of Isoprene in Yarrowia

I. Construction of Vectors for Expression of the Kudzu Isoprene Synthase in Yarrowia lipolytica.

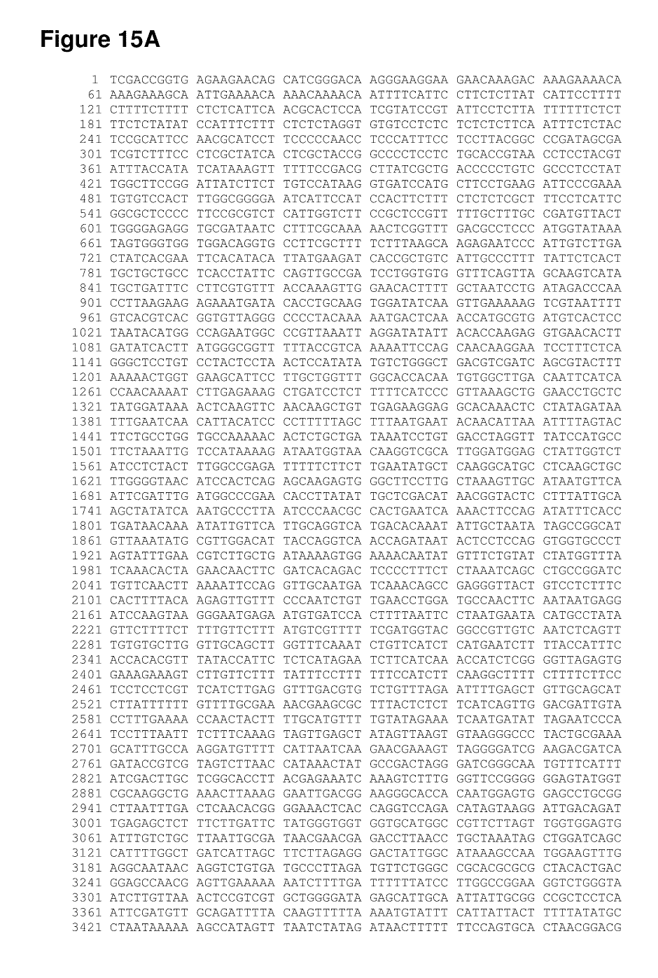

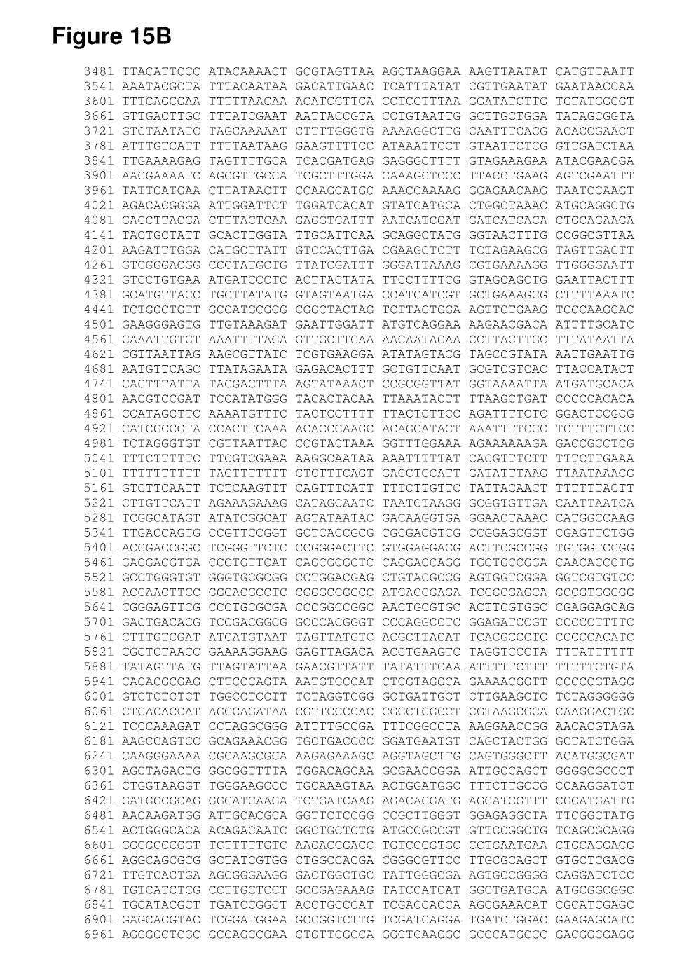

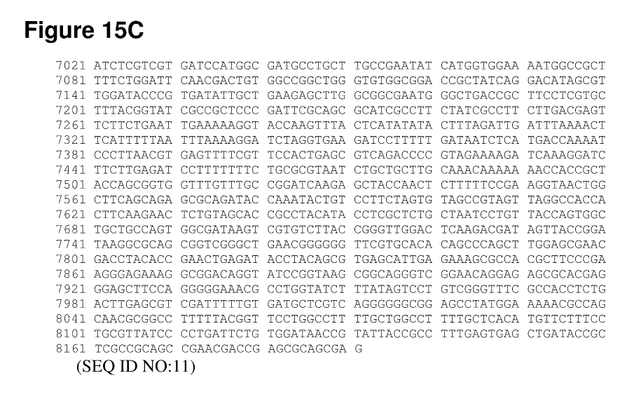

The starting point for the construction of vectors for the expression of the kudzu isoprene synthase gene in Yarrowia lipolytica was the vector pSPZ1(MAP29Spb). The complete sequence of this vector (SEQ ID No:11) is shown in FIGS. 15A-15C.

The following fragments were amplified by PCR using chromosomal DNA of a Y. lipolytica strain GICC 120285 as the template: a promotorless form of the URA3 gene, a fragment of 18S ribosomal RNA gene, a transcription terminator of the Y. lipolytica XPR2 gene and two DNA fragments containing the promoters of XPR2 and ICL1 genes. The following PCR primers were used:

| | |
|---|---|
| ICL1 3 | 2 |

(SEQ ID NO: 69)

5′-GGTGAATTCAGTCTACTGGGGATTCCCAAATCTATATATACTGCAGG
TGAC
ICL1 5

(SEQ ID NO: 70)

5′-GCAGGTGGGAAACTATGCACTCC
XPR 3

(SEQ ID NO: 71)

5′-CCTGAATTCTGTTGGATTGGAGGATTGGATAGTGGG
XPR 5

(SEQ ID NO: 72)

5′-GGTGTCGACGTACGGTCGAGCTTATTGACC
XPRT3

(SEQ ID NO: 73)

5′-GGTGGGCCCGCATTTTGCCACCTACAAGCCAG
XPRT 5

(SEQ ID NO: 74)

5′-GGTGAATTCTAGAGGATCCCAACGCTGTTGCCTACAACGG
Y18S3

(SEQ ID NO: 75)

5′-GGTGCGGCCGCTGTCTGGACCTGGTGAGTTTCCCCG
Y18S 5

(SEQ ID NO: 76)

5′-GGTGGGCCCATTAAATCAGTTATCGTTTATTTGATAG
YURA3

(SEQ ID NO: 77)

5′-GGTGACCAGCAAGTCCATGGGTGGTTTGATCATGG
YURA 50

(SEQ ID NO: 78)

5′-GGTGCGGCCGCCTTTGGAGTACGACTCCAACTATG
YURA 51

(SEQ ID NO: 79)

5′-GCGGCCGCAGACTAAATTTATTTCAGTCTCC

For PCR amplification the PfuUltraII polymerase (Stratagene), supplier-provided buffer and dNTPs, 2.5 μM primers and the indicated template DNA were used as per the manufacturer's instructions. The amplification was done using the following cycle: 95° C. for 1 min; 34× (95° C. for 30 sec; 55° C. for 30 sec; 72° C. for 3 min) and 10 min at 72° C. followed by a 4° C. incubation.

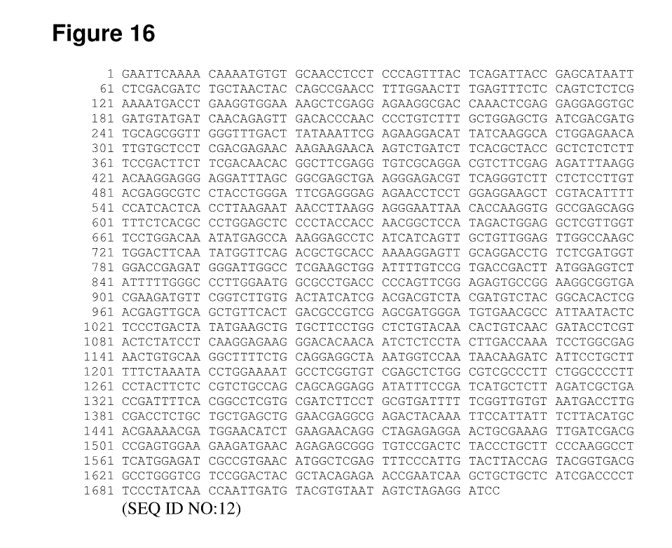

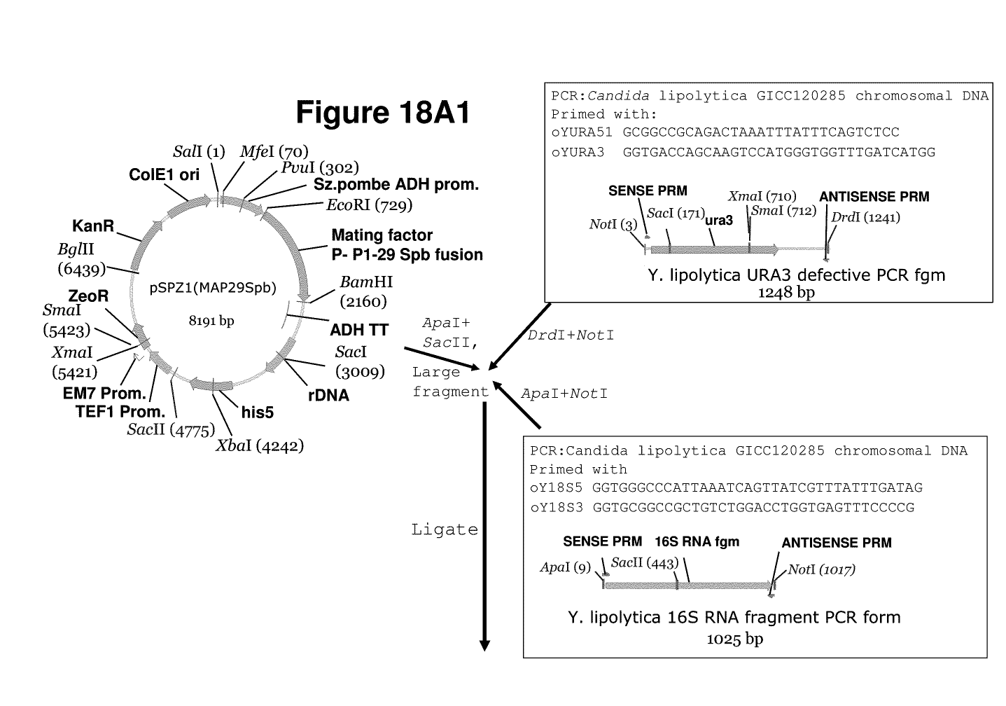

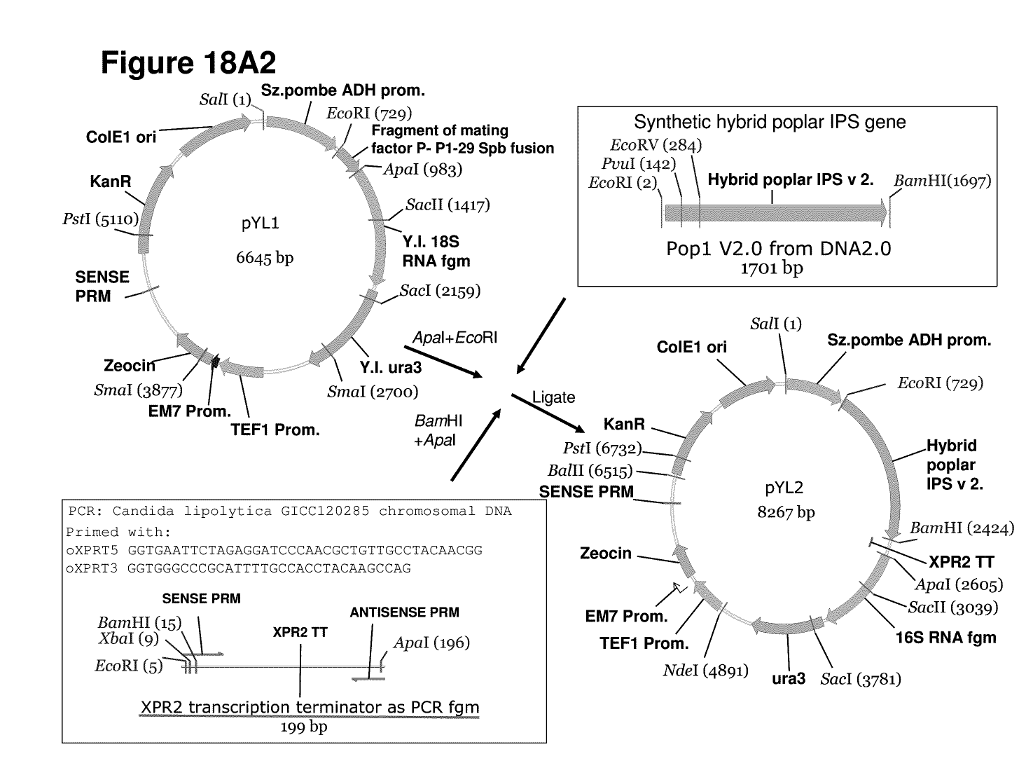

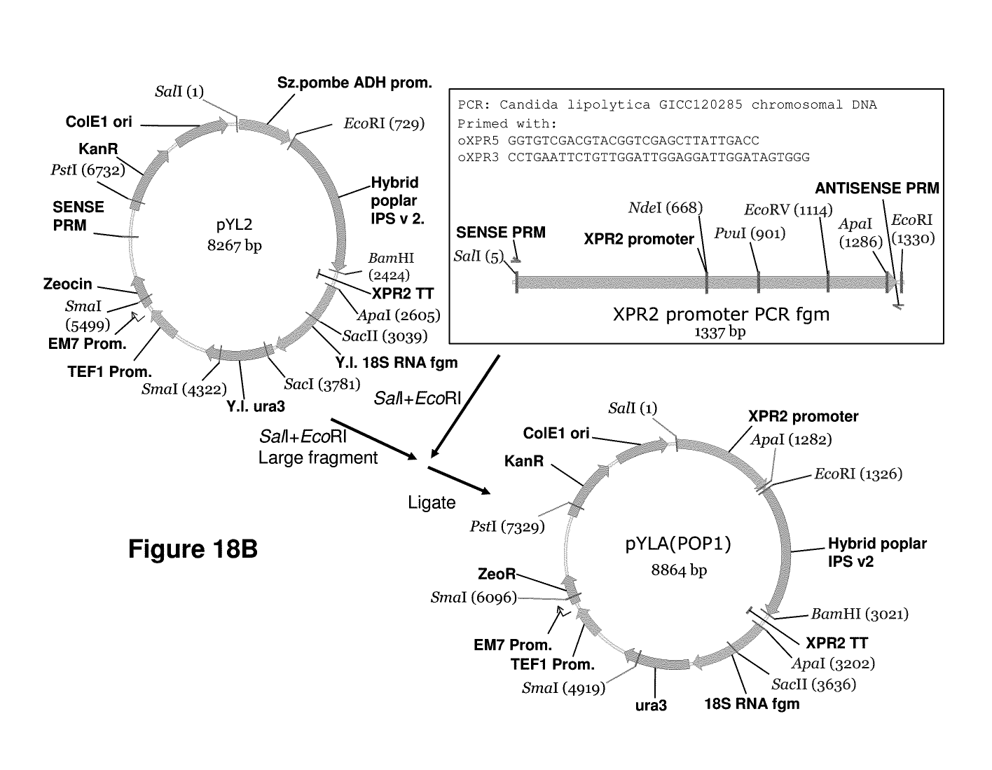

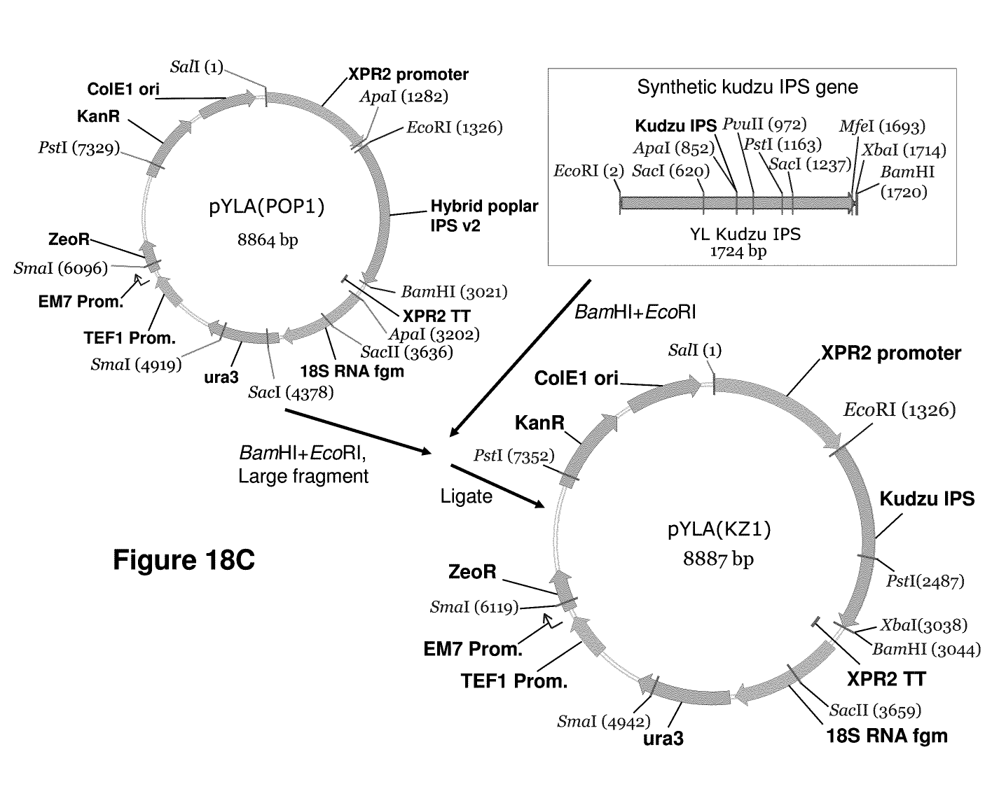

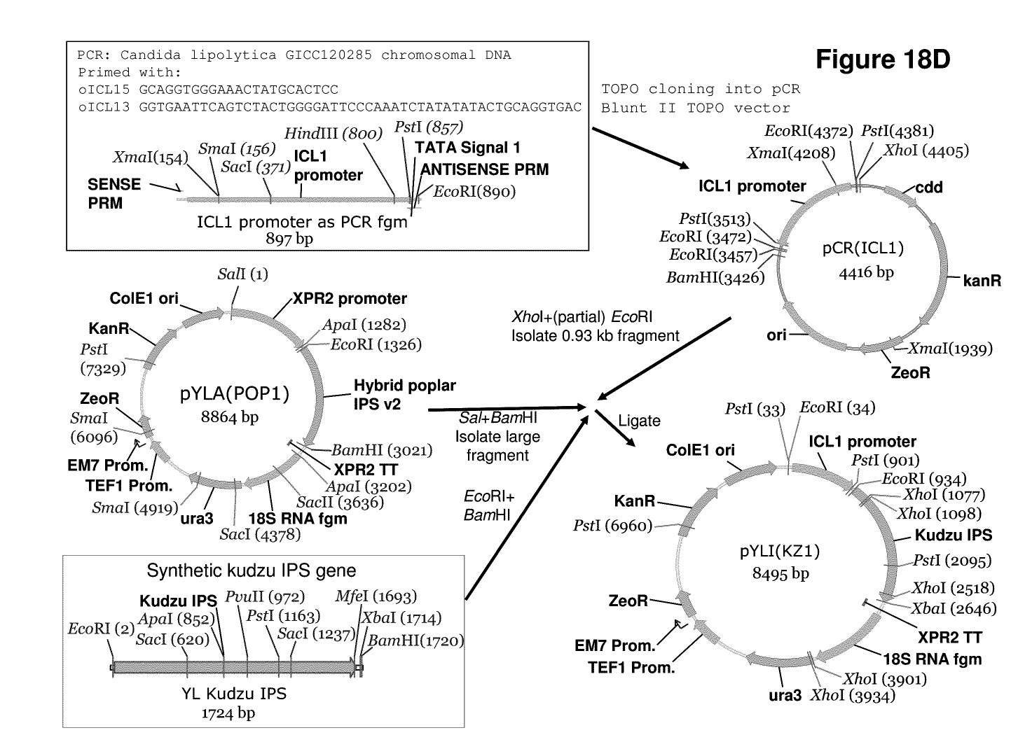

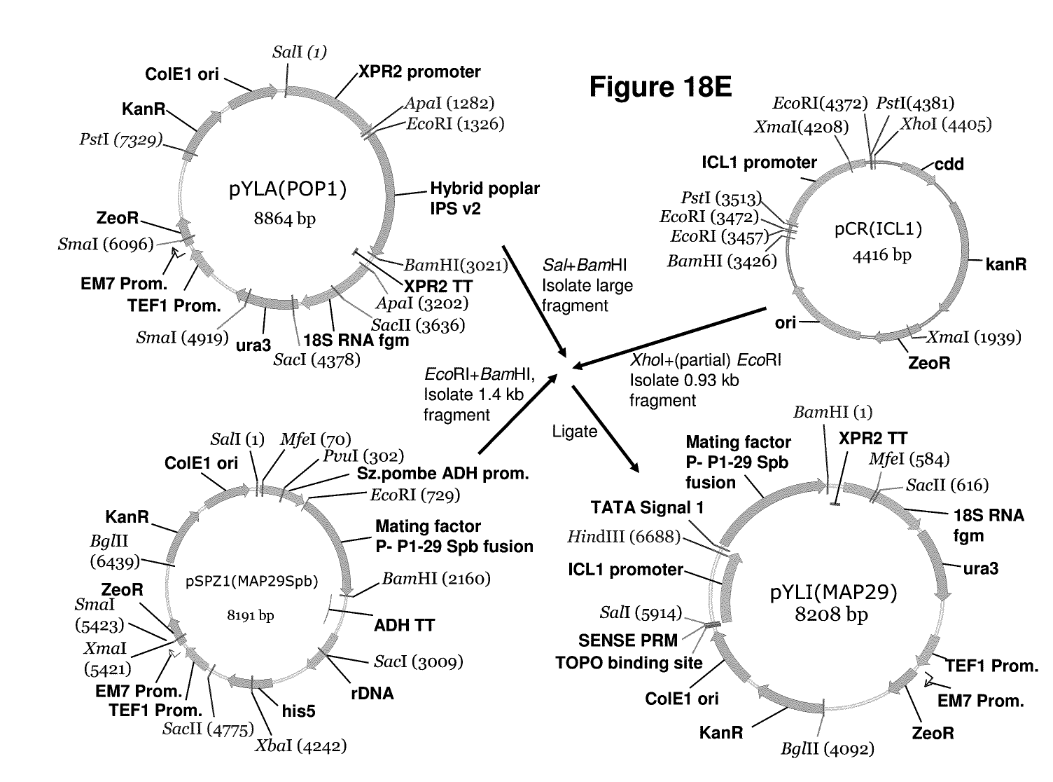

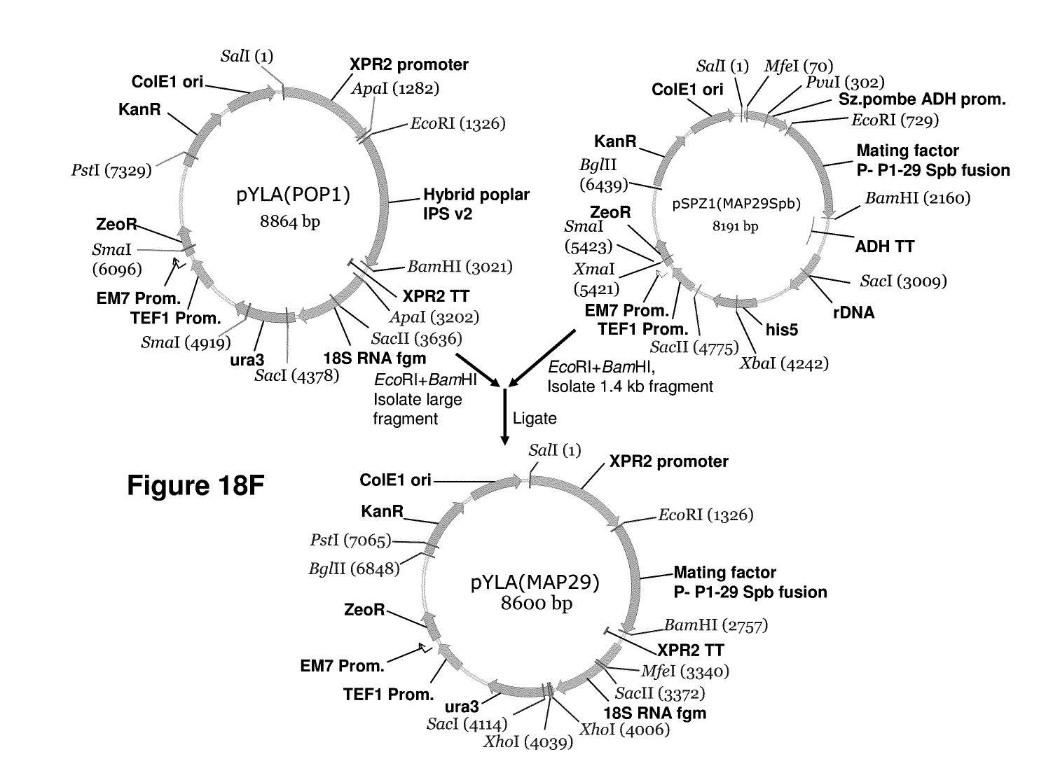

Synthetic DNA molecules encoding the kudzu isoprene synthase gene, codon-optimized for expression in Yarrowia, was obtained from DNA 2.0 (FIG. 16; SEQ ID NO:12). Full detail of the construction scheme of the plasmids pYLA(KZ1) and pYLI(KZ1) carrying the synthetic kudzu isoprene synthase gene under control of XPR2 and ICL1 promoters respectively is presented in FIGS. 18A-18F. Control plasmids in which a mating factor gene (MAP29) is inserted in place of an isoprene synthase gene were also constructed (FIGS. 18E and 18F).

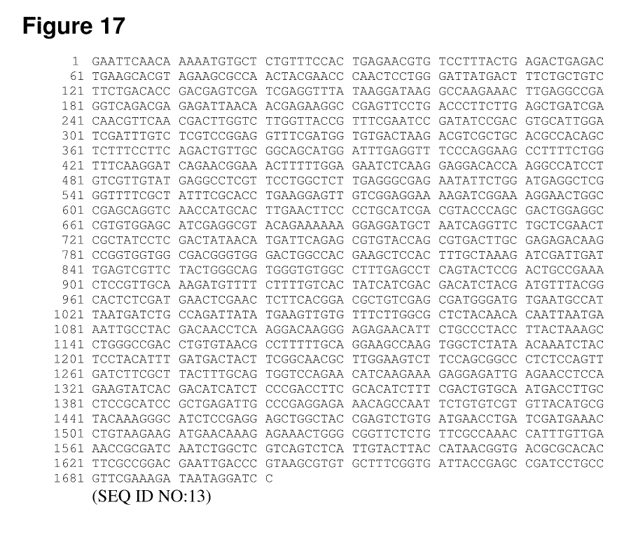

A similar cloning procedure can be used to express a poplar (Populus alba×Populus tremula) isoprene synthase gene. The sequence of the poplar isoprene is described in Miller B. et al. (2001) Planta 213, 483-487 and shown in FIG. 17 (SEQ ID NO:13). A construction scheme for the generation the plasmids pYLA(POP1) and pYLI(POP1) carrying synthetic poplar isoprene synthase gene under control of XPR2 and ICL1 promoters respectively is presented in FIGS. 18A and B.

II. Production of Isoprene by Recombinant Strains of Y. lipolytica.

Vectors pYLA(KZ1), pYLI(KZ1), pYLA(MAP29) and pYLI(MAP29) were digested with SacII and used to transform the strain Y. lipolytica CLIB 122 by a standard lithium acetate/polyethylene glycol procedure to uridine prototrophy. Briefly, the yeast cells grown in YEPD (1% yeast extract, 2% peptone, 2% glucose) overnight, were collected by centrifugation (4000 rpm, 10 min), washed once with sterile water and suspended in 0.1 M lithium acetate, pH 6.0. Two hundred μl aliquots of the cell suspension were mixed with linearized plasmid DNA solution (10-20 μg), incubated for 10 minutes at room temperature and mixed with 1 ml of 50% PEG 4000 in the same buffer. The suspensions were further incubated for 1 hour at room temperature followed by a 2 minutes heat shock at 42° C. Cells were then plated on SC his leu plates (0.67% yeast nitrogen base, 2% glucose, 100 mg/L each of leucine and histidine). Transformants appeared after 3-4 days of incubation at 30° C.

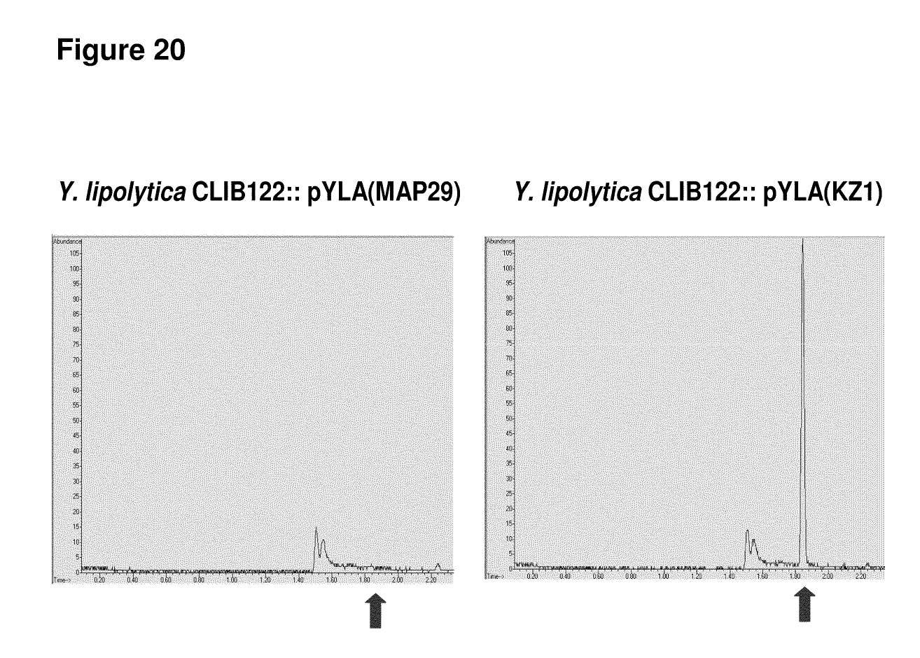

Three isolates from the pYLA(KZ1) transformation, three isolates from the pYLI(KZ1) transformation, two isolates from the pYLA(MAP29) transformation and two isolates from the pYLI(MAP29) transformation were grown for 24 hours in YEP7 medium (1% yeast extract, 2% peptone, pH 7.0) at 30° C. with shaking. Cells from 10 ml of culture were collected by centrifugation, resuspended in 3 ml of fresh YEP7 and placed into 15 ml screw cap vials. The vials were incubated overnight at room temperature with gentle (60 rpm) shaking. Isoprene content in the headspace of these vials was analyzed by gas chromatography using mass-spectrometric detector as described in Example 13. All transformants obtained with pYLA(KZ1) and pYLI(KZ1) produced readily detectable amounts of isoprene (0.5 μg/L to 1 μg/L, FIG. 20). No isoprene was detected in the headspace of the control strains carrying phytase gene instead of an isoprene synthase gene.

### Example 19

### Production of Isoprene in E. coli Expressing Kudzu Isoprene Synthase and idi, or dxs, or idi and dxs

I. Construction of Vectors Encoding Kudzu Isoprene Synthase and idi, or dxs, or idi and dxs for the Production of Isoprene in E. coli
i) Construction of pTrcKudzuKan

The bla gene of pTrcKudzu (described in Example 13) was replaced with the gene conferring kanamycin resistance. To remove the bla gene, pTrcKudzu was digested with BspHI, treated with Shrimp Alkaline Phosphatase (SAP), heat killed at 65° C., then end-filled with Klenow fragment and dNTPs. The 5 kbp large fragment was purified from an agarose gel and ligated to the kan^{r} gene which had been PCR amplified from pCR-Blunt-II-TOPO using primers MCM22 5′-GATCAAGCTTAACCGGAATTGCCAGCTG (SEQ ID NO:14) and MCM23 5′-GATCCGATCGTCAGAAGAACTCGTCAAGAAGGC (SEQ ID NO:15), digested with HindlII and PvuI, and end-filled. A transformant carrying a plasmid conferring kanamycin resistance (pTrcKudzuKan) was selected on LA containing kanamycin 50 μg/ml.

ii) Construction of pTrcKudzu yIDI Kan

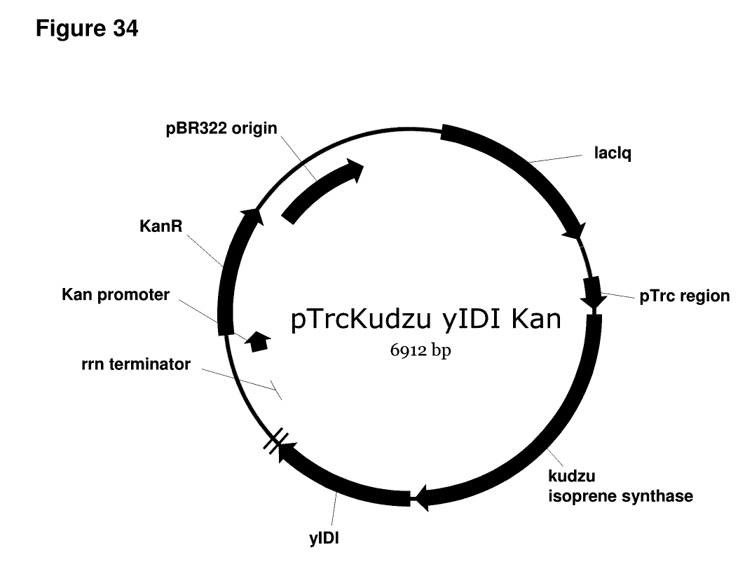

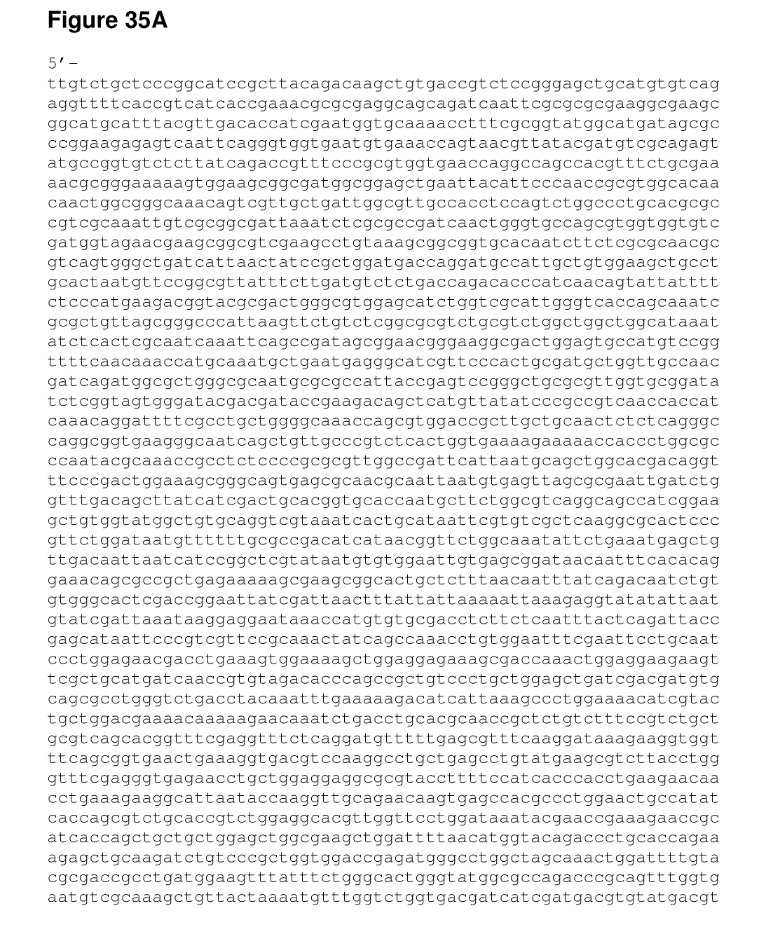

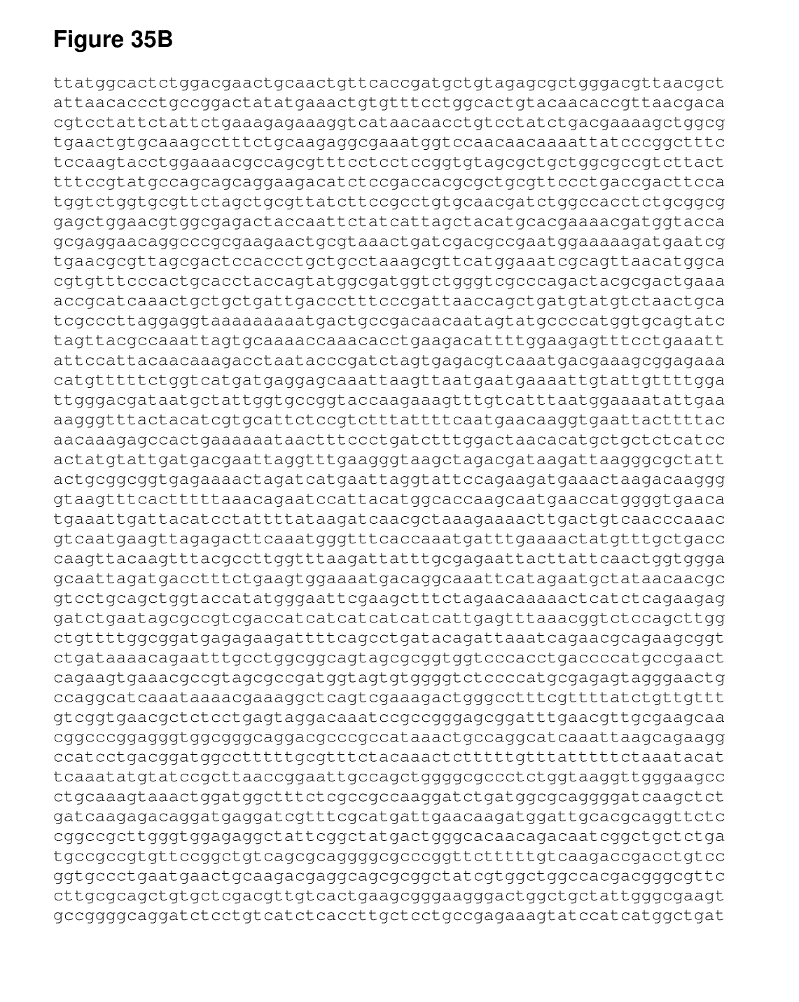

pTrcKudzuKan was digested with PstI, treated with SAP, heat killed and gel purified. It was ligated to a PCR product encoding idi from S. cerevisiae with a synthetic RBS. The primers for PCR were NsiI-YIDI 1 F 5′-CATCAATGCATCGCCCTTAGGAGGTAAAAAAAAATGAC (SEQ ID NO:16) and PstI-YIDI 1 R 5′-CCTTCTGCAGGACGCGTTGTTATAGC (SEQ ID NO:17); and the template was S. cerevisiae genomic DNA. The PCR product was digested with NsiI and PstI and gel purified prior to ligation. The ligation mixture was transformed into chemically competent TOP10 cells and selected on LA containing 50 μg/ml kanamycin. Several transformants were isolated and sequenced and the resulting plasmid was called pTrcKudzu-yIDI(kan) (FIGS. 34 and 35A-35C).

iii) Construction of pTrcKudzu DXS Kan

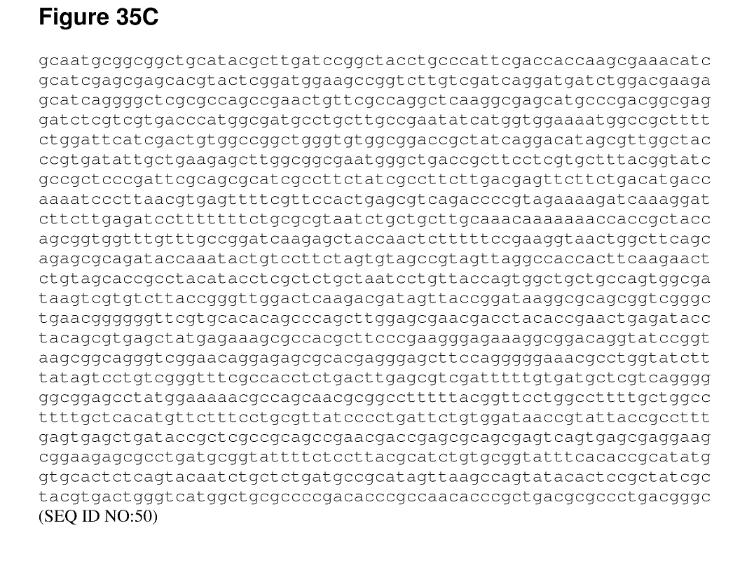

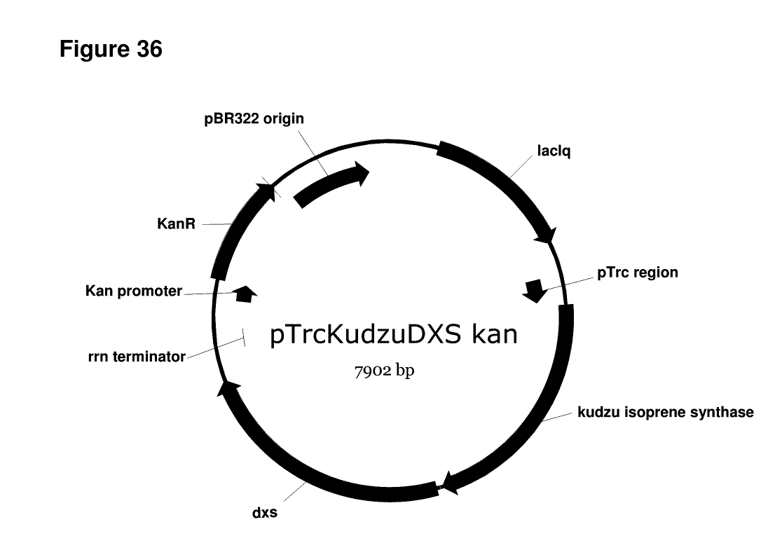

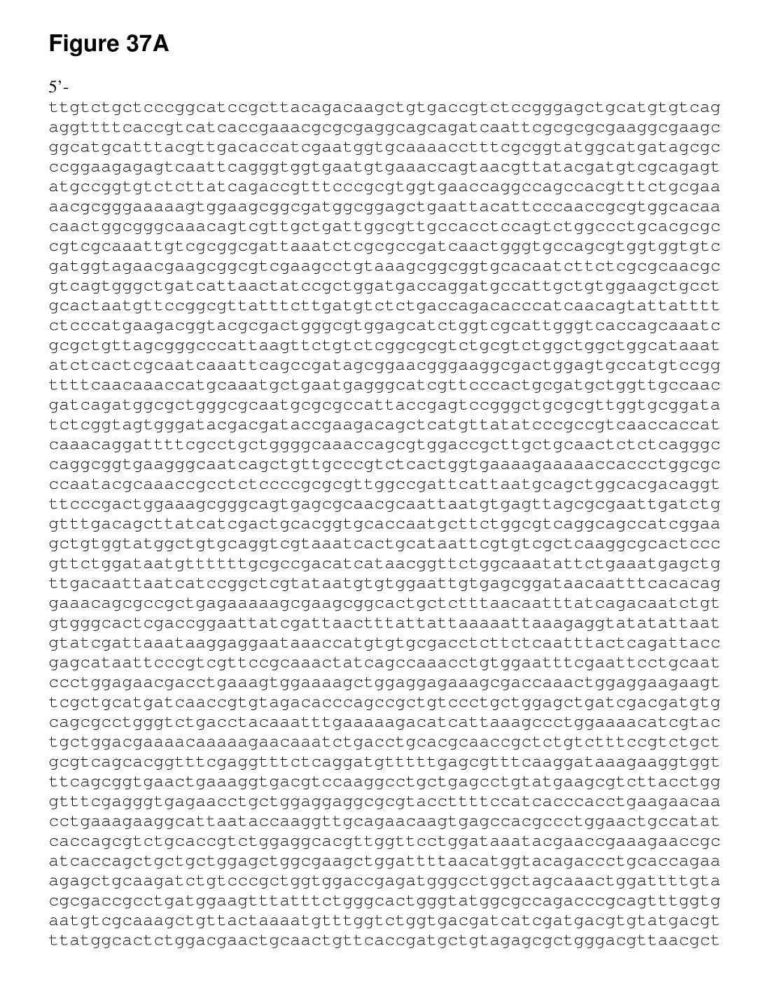

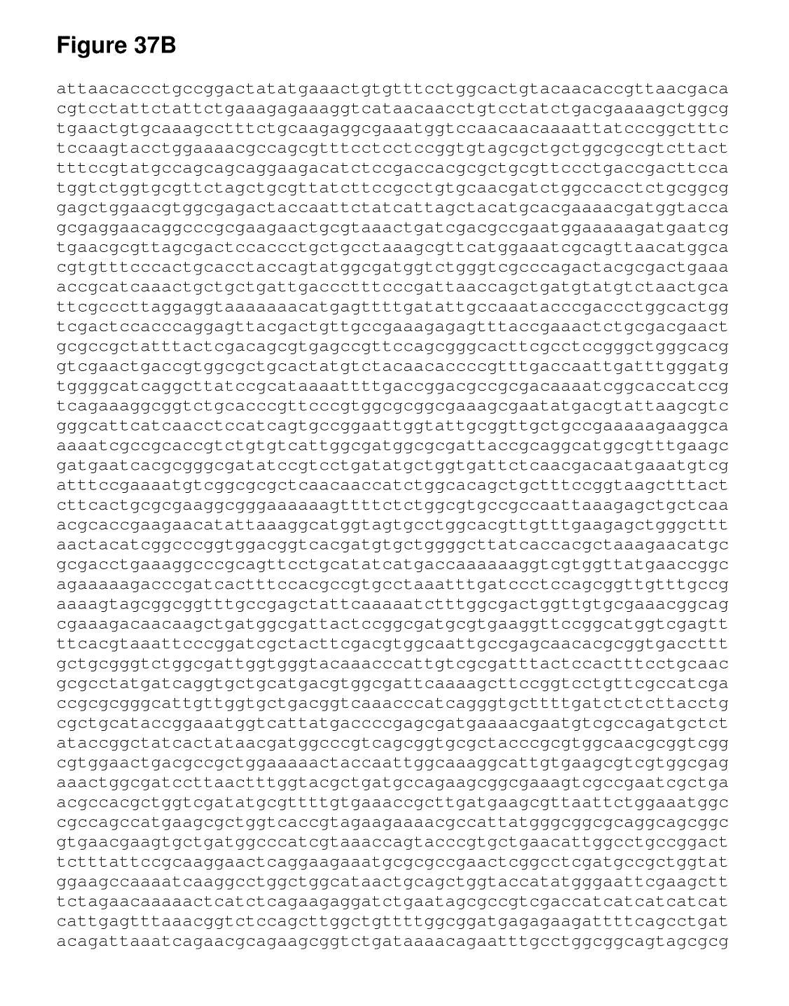

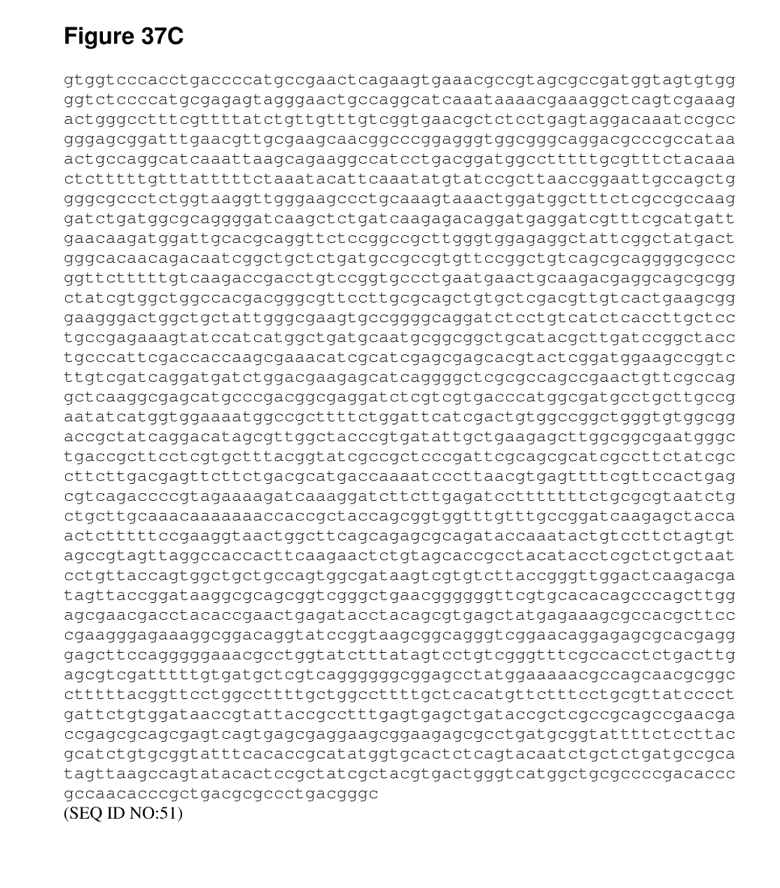

Plasmid pTrcKudzuKan was digested with PstI, treated with SAP, heat killed and gel purified. It was ligated to a PCR product encoding dxs from E. coli with a synthetic RBS. The primers for PCR were MCM 13 5′-GATCATGCATTCGCCCTTAGGAGGTAAAAAAACATGAGTTTTGATATTGCCAAATACCCG (SEQ ID NO:18) and MCM14 5′-CATGCTGCAGTTATGCCAGCCAGGCCTTGAT (SEQ ID NO:19); and the template was E. coli genomic DNA. The PCR product was digested with NsiI and PstI and gel purified prior to ligation. The resulting transformation reaction was transformed into TOP10 cells and selected on LA with kanamycin 50 μg/ml. Several transformants were isolated and sequenced and the resulting plasmid was called pTrcKudzu-DXS(kan) (FIGS. 36 and 37A-37C).

iv) Construction of pTrcKudzu-yIDI-dxs (kan)

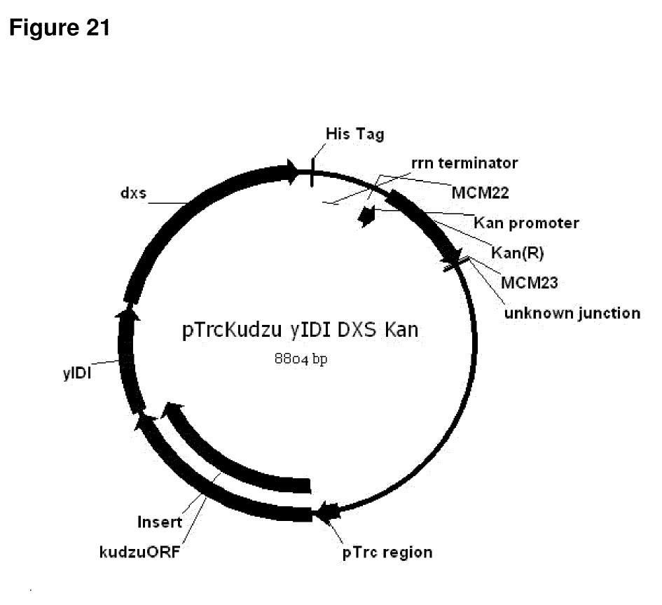

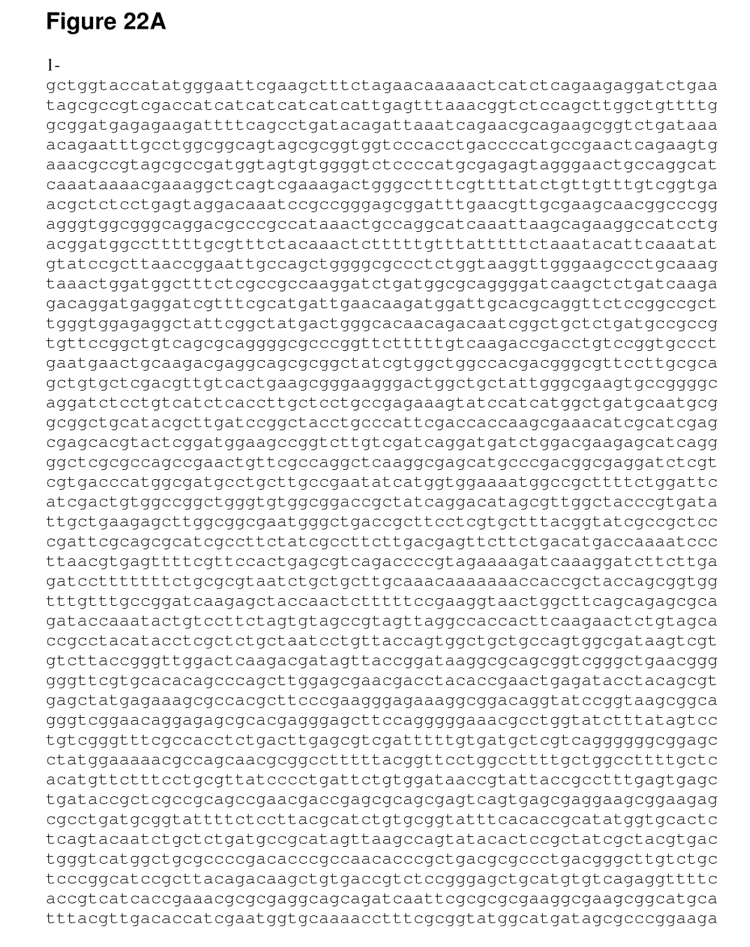

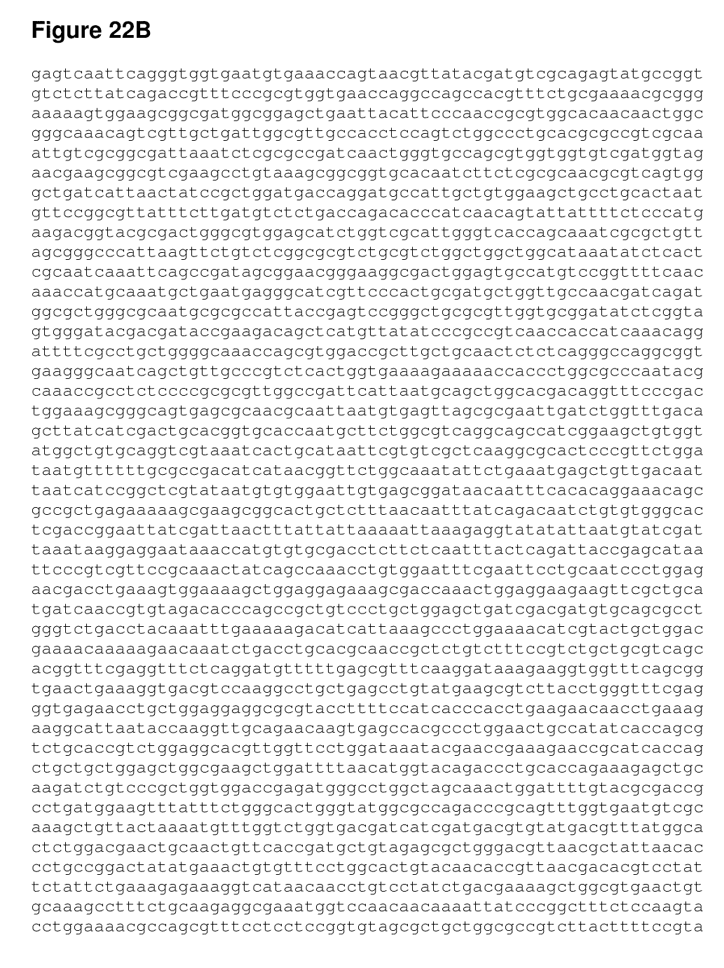

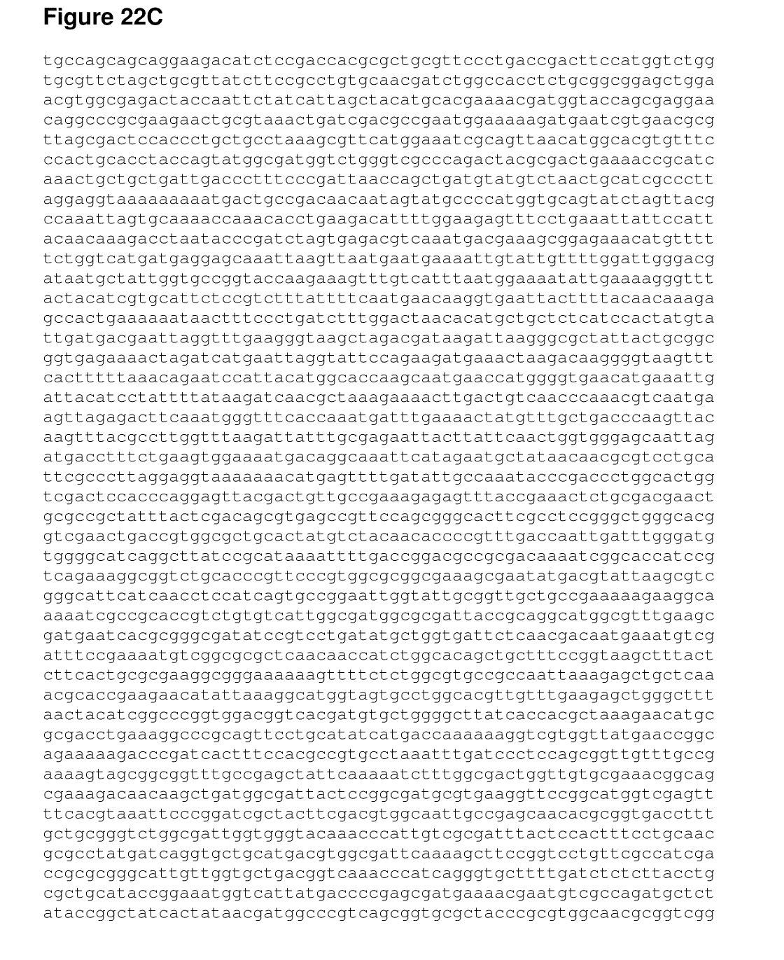

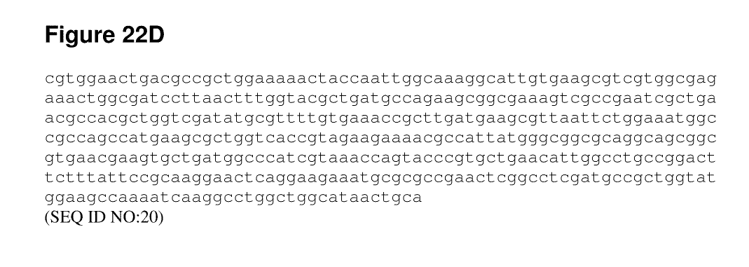

pTrcKudzu-yIDI(kan) was digested with PstI, treated with SAP, heat killed and gel purified. It was ligated to a PCR product encoding E. coli dxs with a synthetic RBS (primers MCM13 5′-GATCATGCATTCGCCCTTAGGAGGTAAAAAAACATGAGTTTTGATATTGCCAAATACCCG (SEQ ID NO:18) and MCM14 5′-CATGCTGCAGTTATGCCAGCCAGGCCTTGAT (SEQ ID NO:19); template TOP10 cells) which had been digested with NsiI and PstI and gel purified. The final plasmid was called pTrcKudzu-yIDI-dxs (kan) (FIGS. 21 and 22A-22D).

### v) Construction of pCL PtrcKudzu

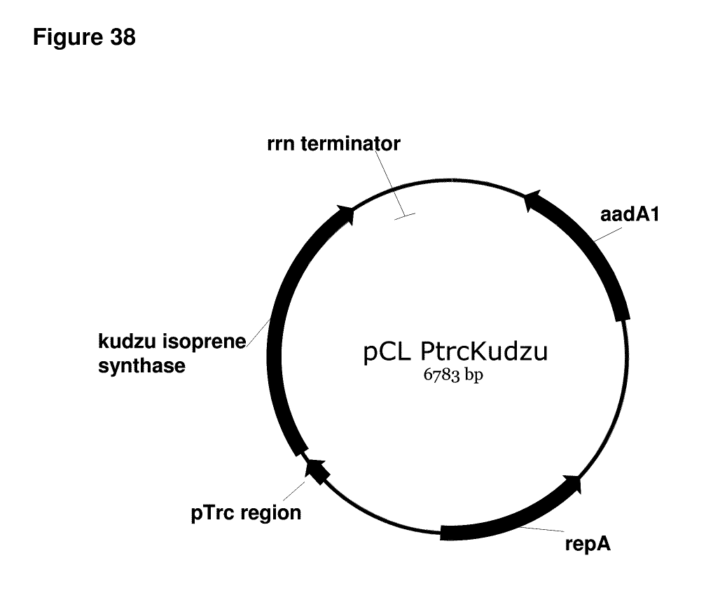

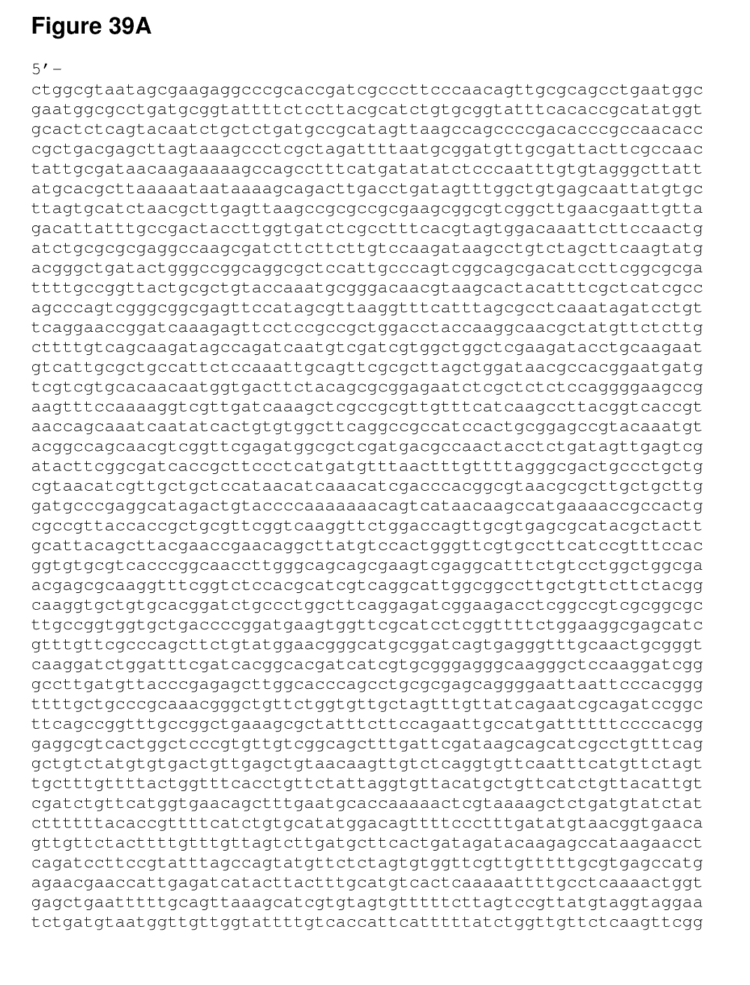

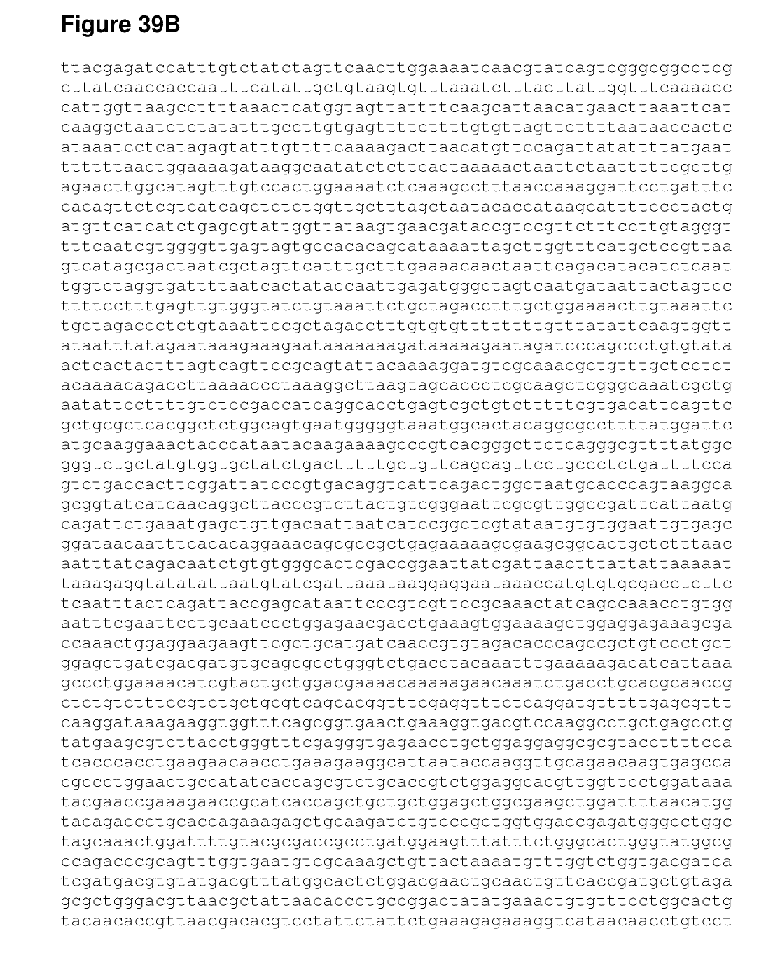

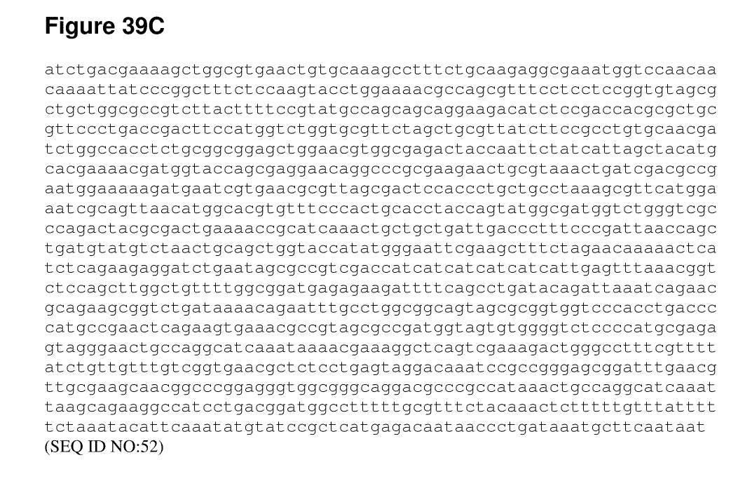

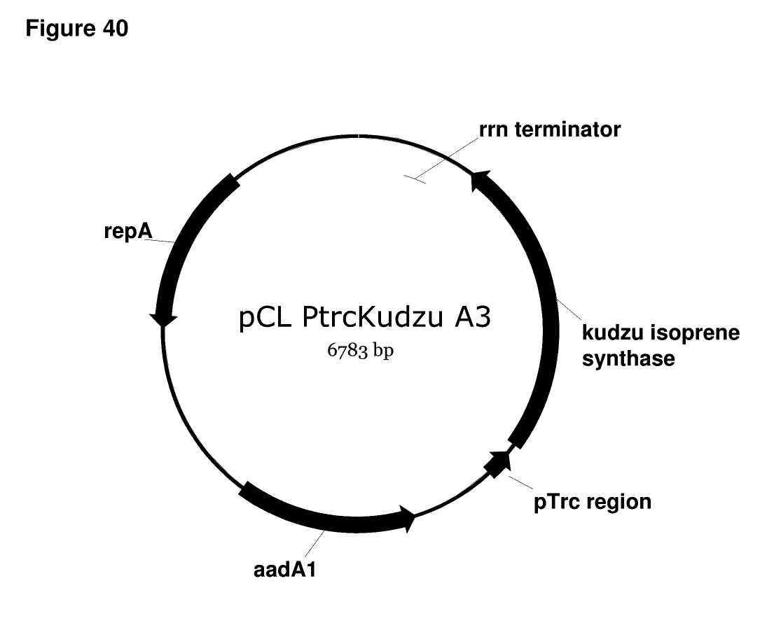

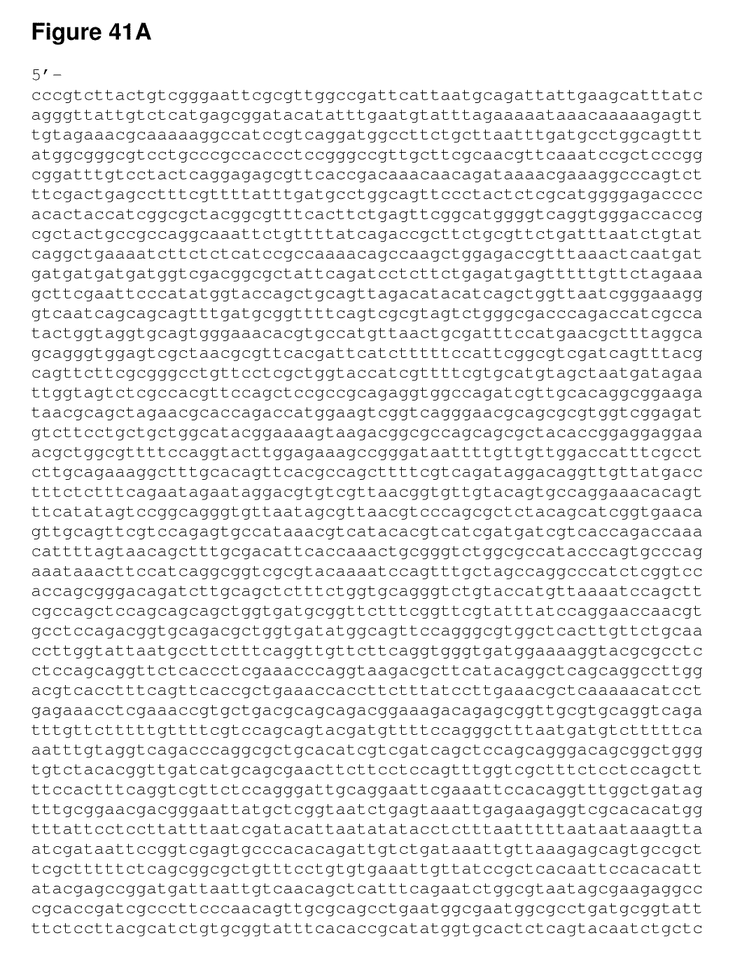

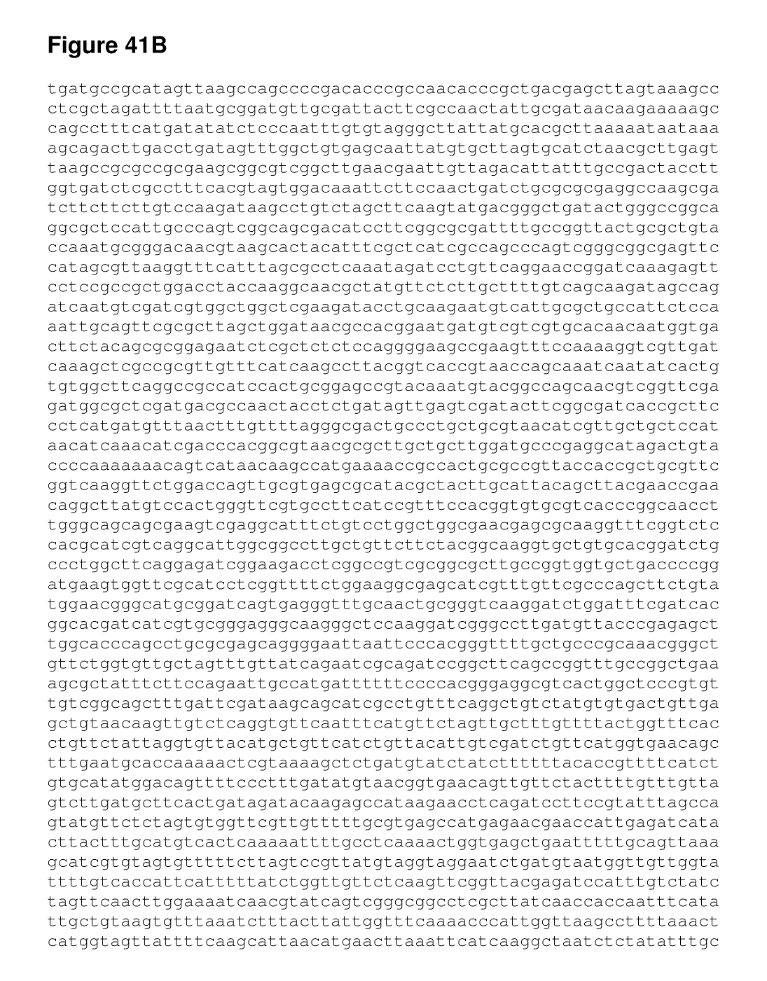

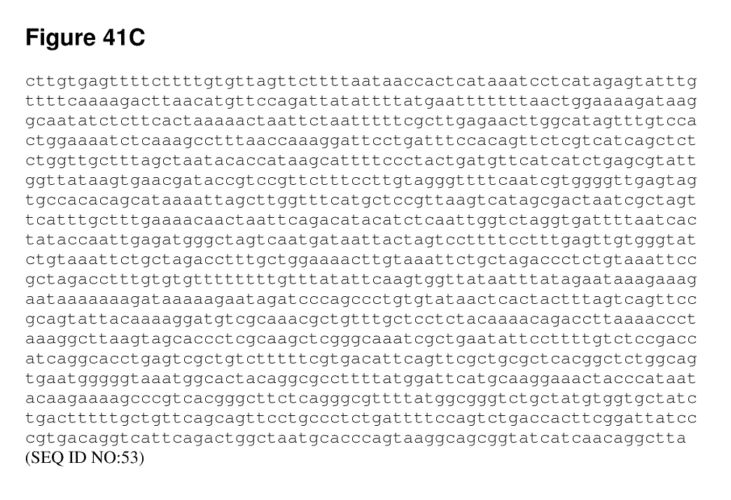

A fragment of DNA containing the promoter, structural gene and terminator from Example 13 above was digested from pTrcKudzu using SspI and gel purified. It was ligated to pCL1920 which had been digested with PvuII, treated with SAP and heat killed. The resulting ligation mixture was transformed into TOP10 cells and selected in LA containing spectinomycin 50 μg/ml. Several clones were isolated and sequenced and two were selected. pCL PtrcKudzu and pCL PtrcKudzu (A3) have the insert in opposite orientations (FIGS. 38-41A-41C).

vi) Construction of pCL PtrcKudzu yIDI

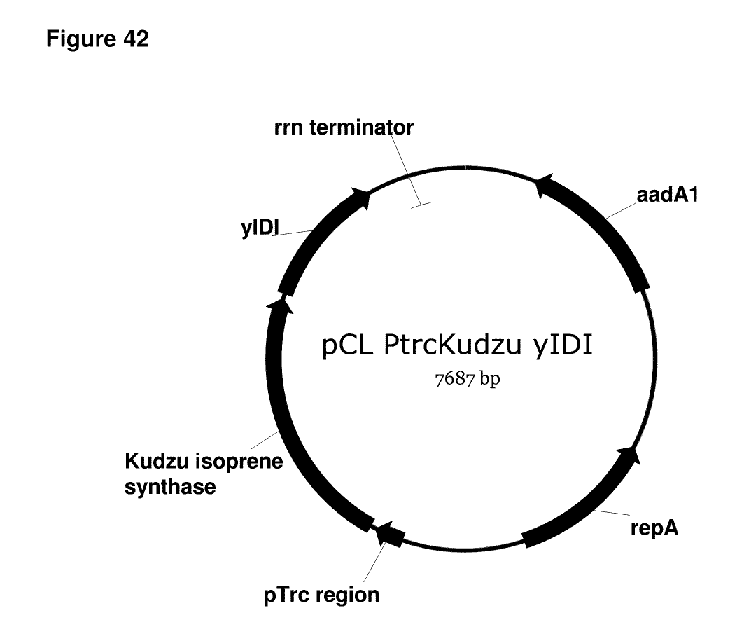

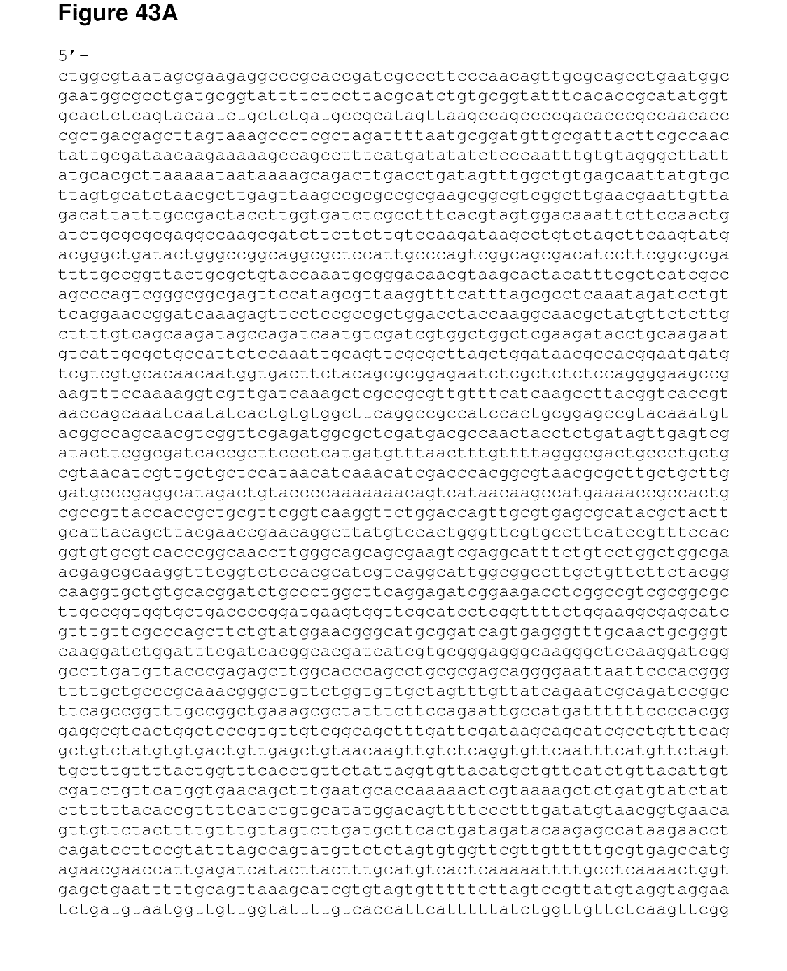

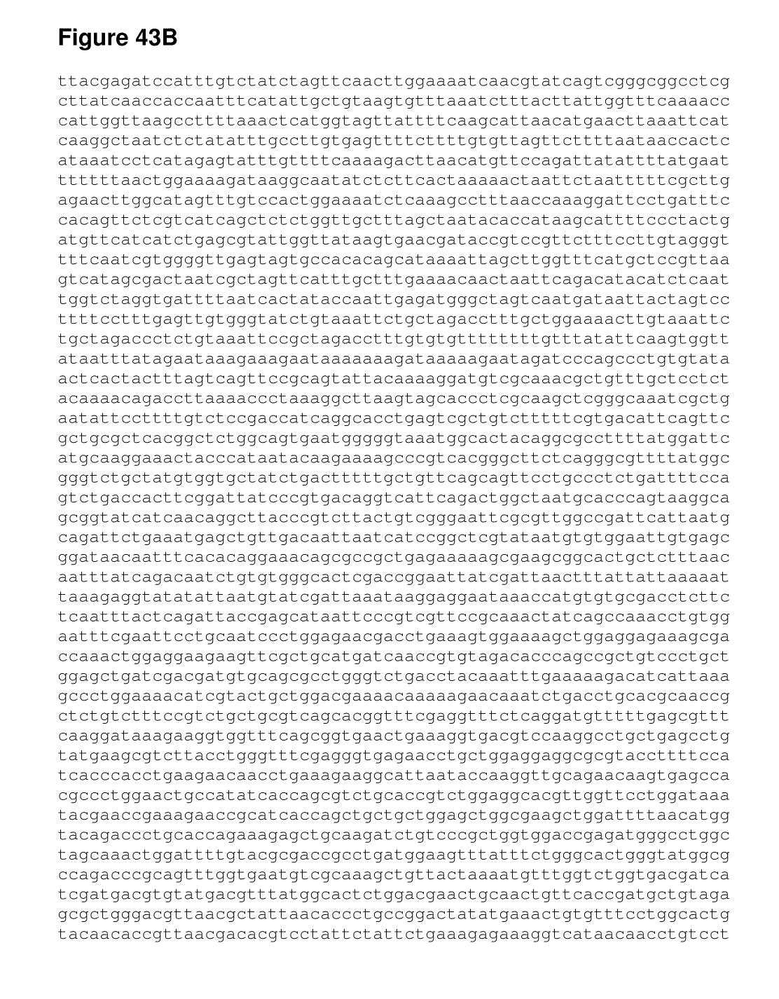

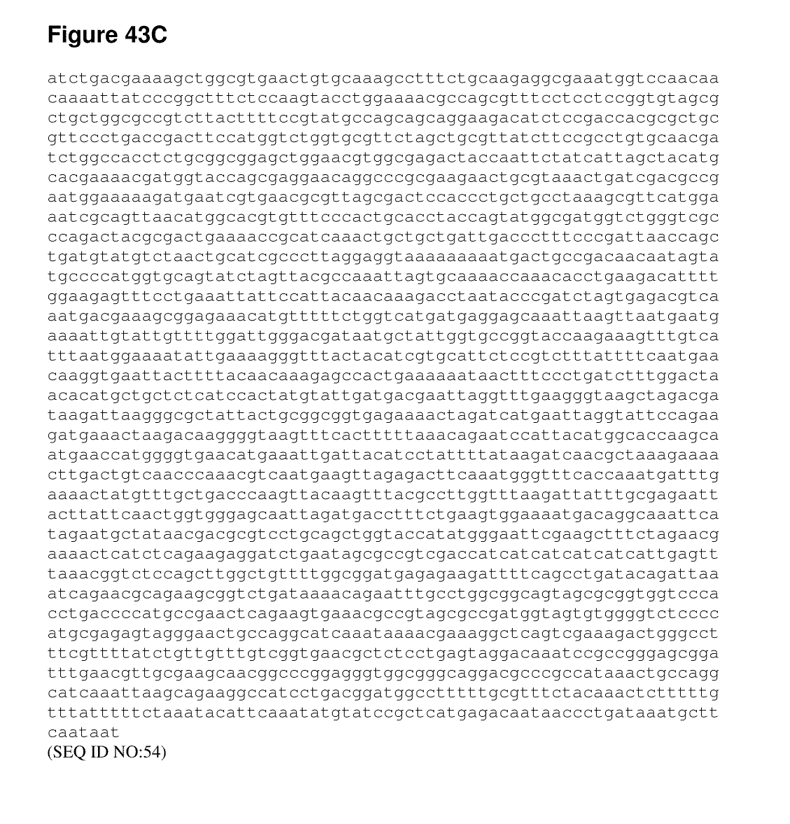

The NsiI-PstI digested, gel purified, IDI PCR amplicon from (ii) above was ligated into pCL PtrcKudzu which had been digested with PstI, treated with SAP, and heat killed. The ligation mixture was transformed into TOP10 cells and selected in LA containing spectinomycin 50 μg/ml. Several clones were isolated and sequenced and the resulting plasmid is called pCL PtrcKudzu yIDI (FIGS. 42 and 43A-43C).

vii) Construction of pCL PtrcKudzu DXS

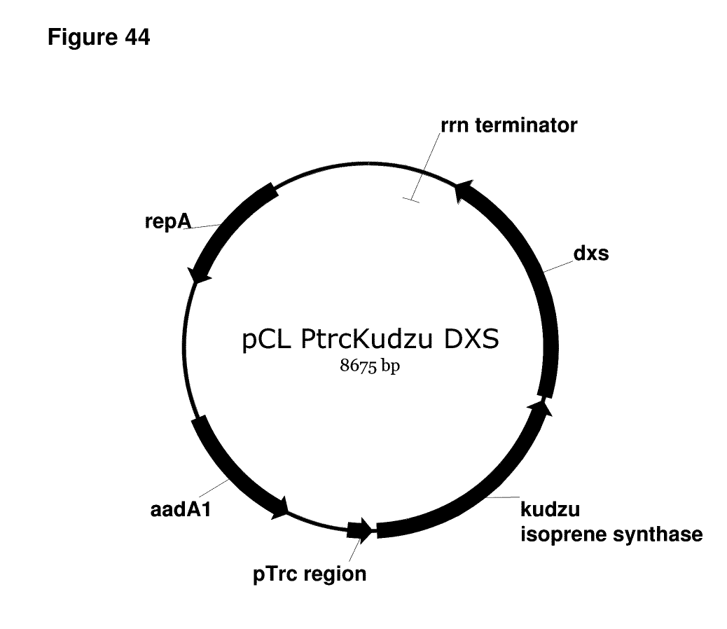

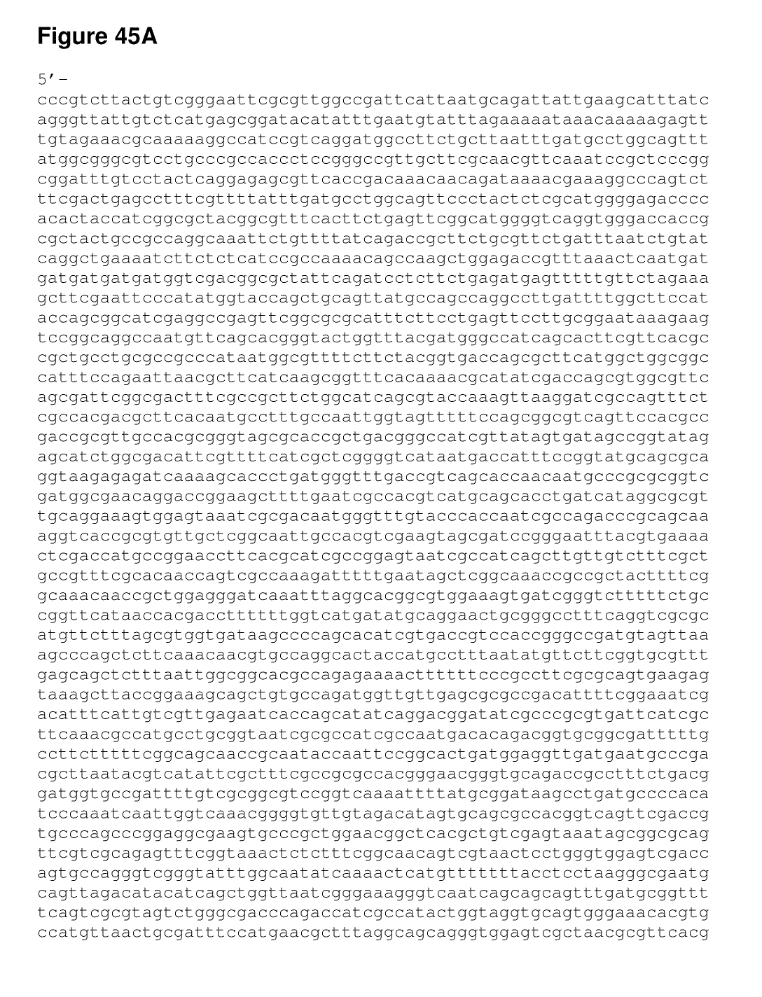

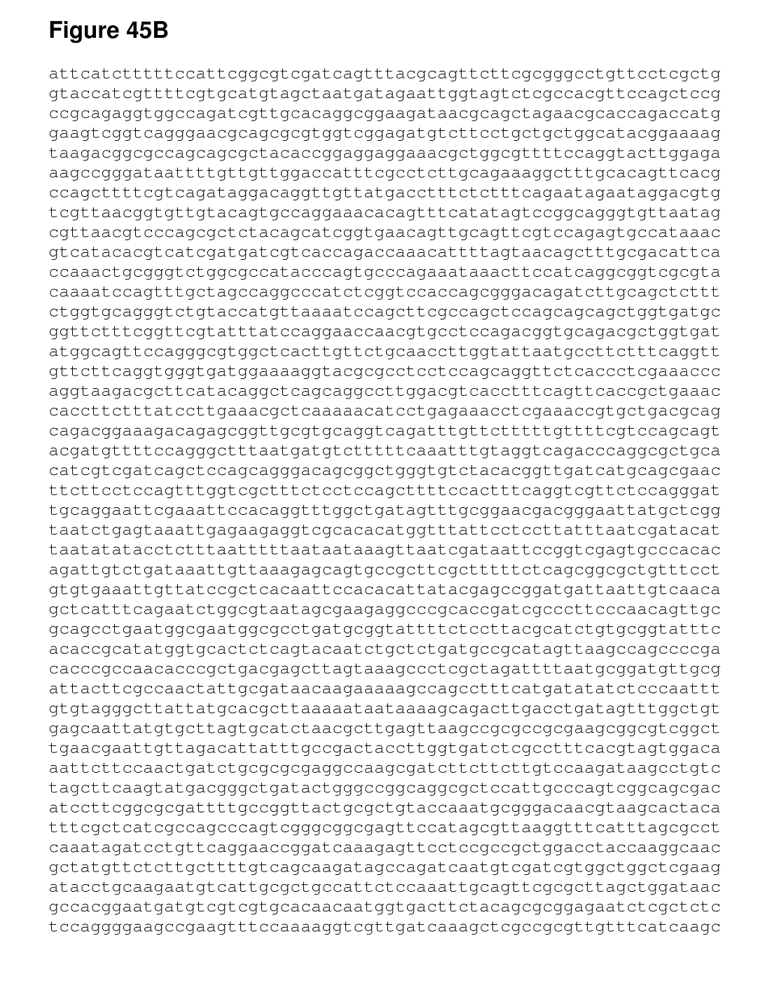

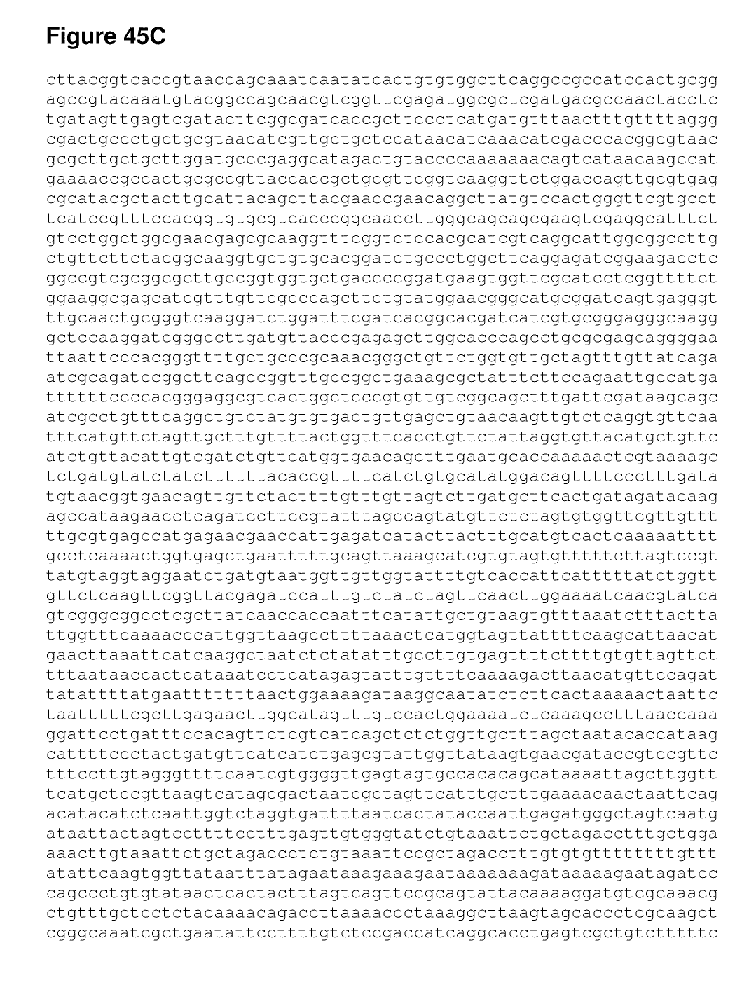

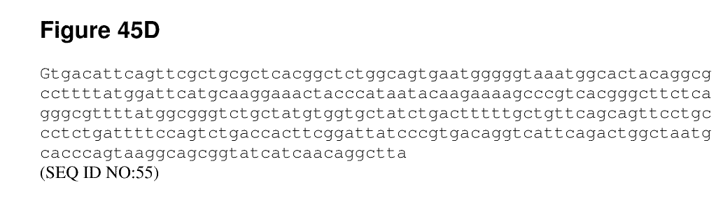

The NsiI-PstI digested, gel purified, DXS PCR amplicon from (iii) above was ligated into pCL PtrcKudzu (A3) which had been digested with PstI, treated with SAP, and heat killed. The ligation mixture was transformed into TOP10 cells and selected in LA containing spectinomycin 50 μg/ml. Several clones were isolated and sequenced and the resulting plasmid is called pCL PtrcKudzu DXS (FIGS. 44 and 45A-45D).

II. Measurement of Isoprene in Headspace from Cultures Expressing Kudzu Isoprene Synthase, idi, and/or dxs at Different Copy Numbers.

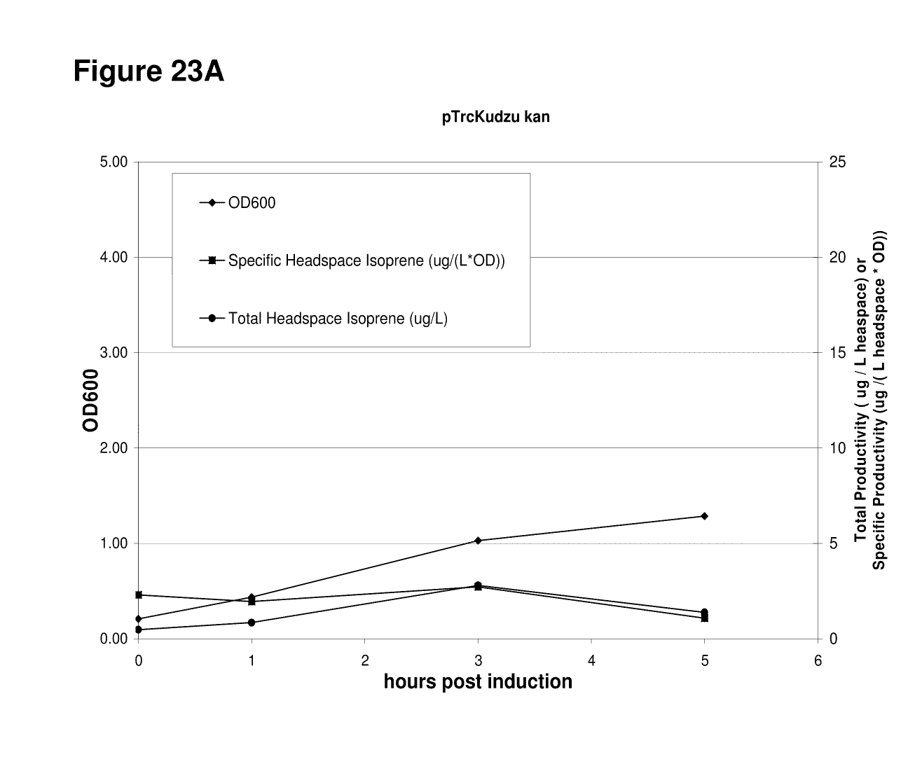

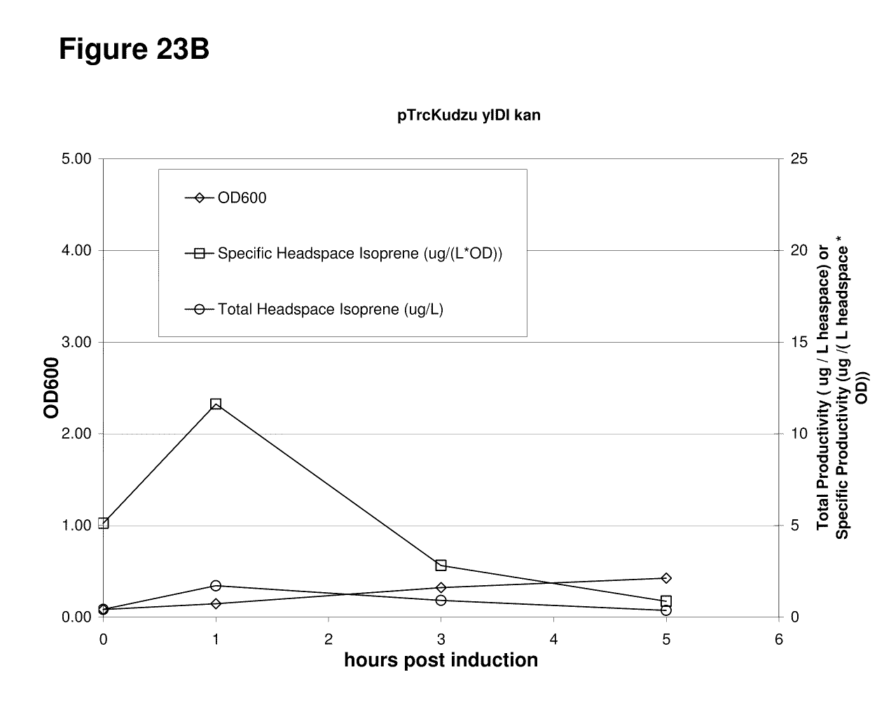

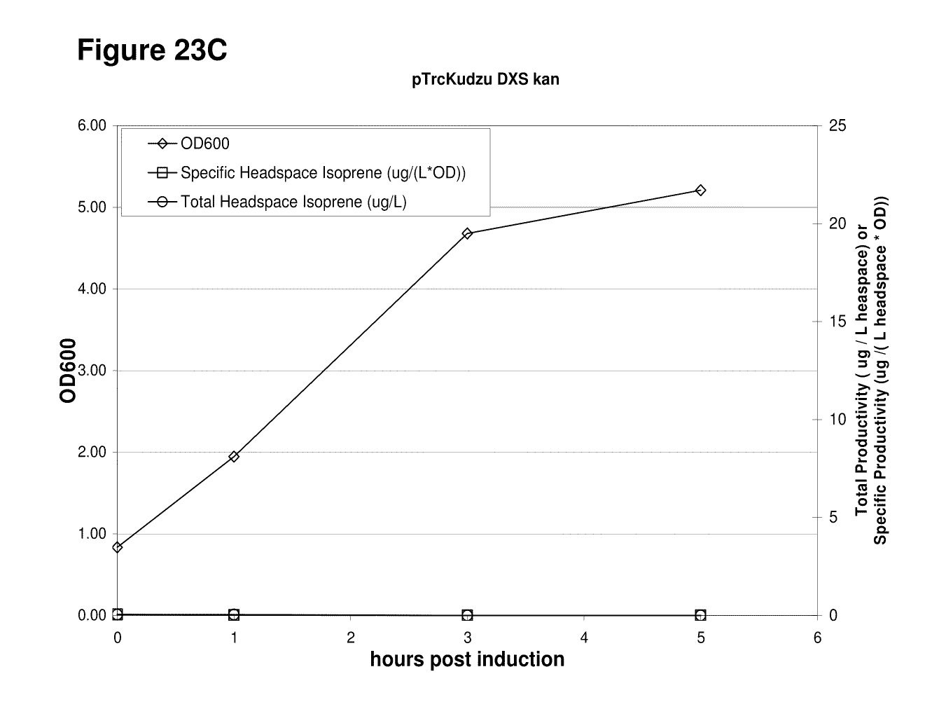

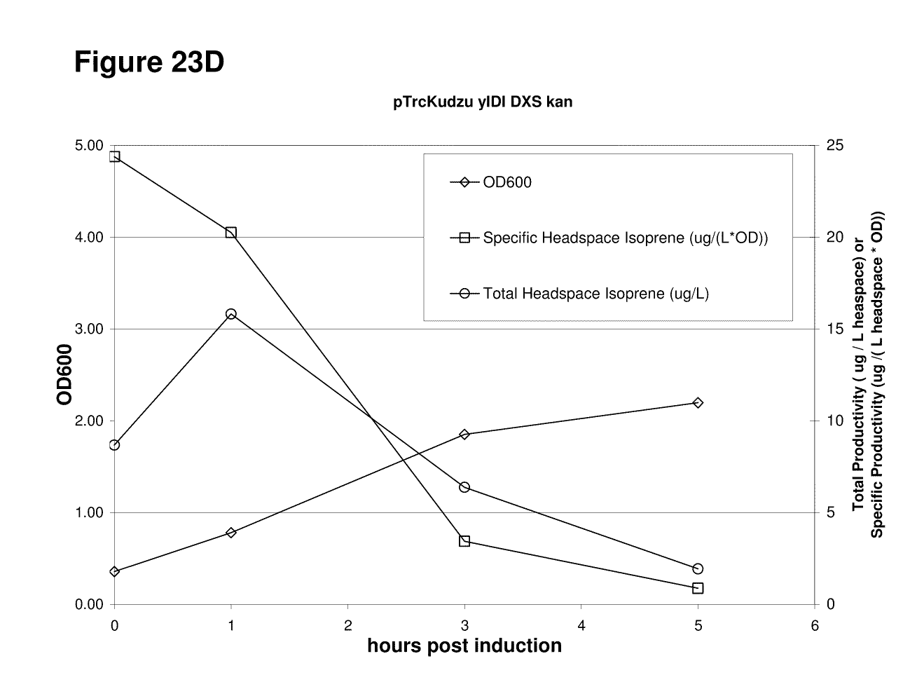

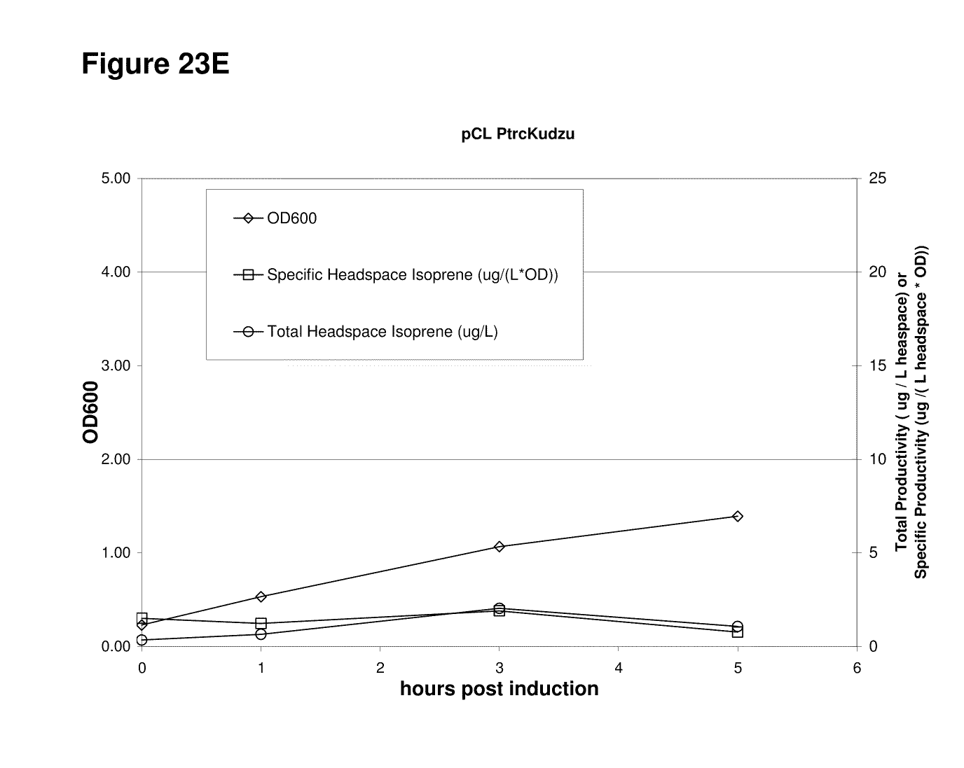

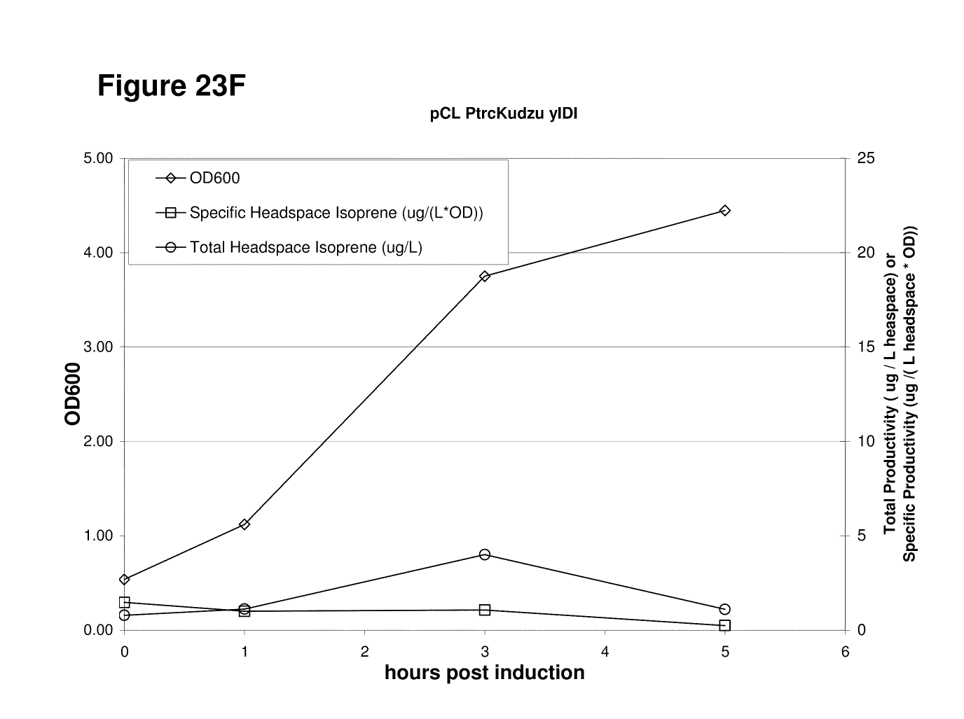

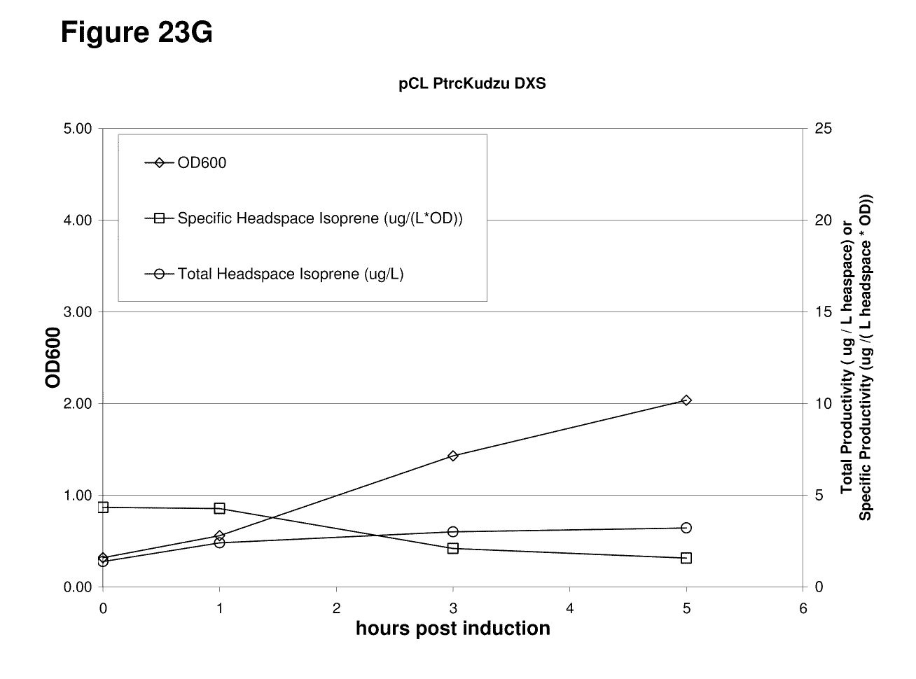

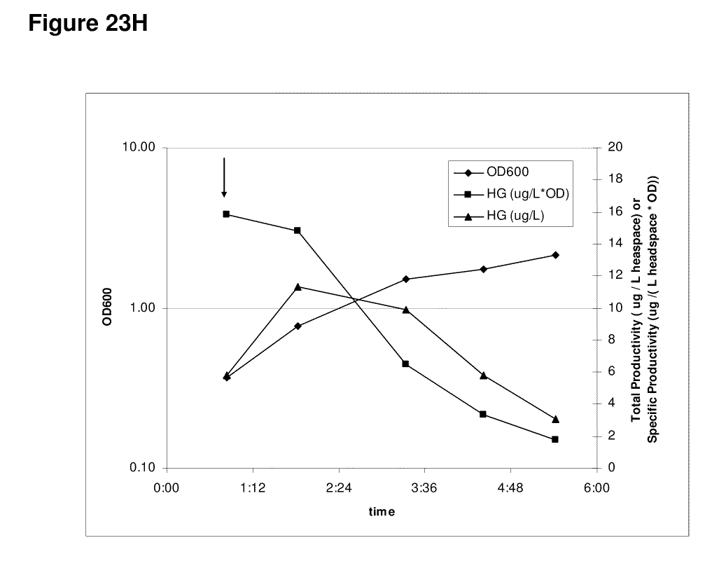

Cultures of E. coli BL21(λDE3) previously transformed with plasmids pTrcKudzu(kan) (A), pTrcKudzu-yIDI kan (B), pTrcKudzu-DXS kan (C), pTrcKudzu-yIDI-DXS kan (D) were grown in LB kanamycin 50 μg/mL. Cultures of pCL PtrcKudzu (E), pCL PtrcKudzu, pCL PtrcKudzu-yIDI (F) and pCL PtrcKudzu-DXS (G) were grown in LB spectinomycin 50 μg/mL. Cultures were induced with 400 μM IPTG at time 0 (OD₆₀₀ approximately 0.5) and samples taken for isoprene headspace measurement (see Example 13). Results are shown in FIG. 23A-23G.

Plasmid pTrcKudzu-yIDI-dxs (kan) was introduced into E. coli strain BL21 by transformation. The resulting strain BL21/pTrc Kudzu IDI DXS was grown overnight in LB containing kanamycin (50 μg/ml) at 20° C. and used to inoculate shake flasks of TM3 (13.6 g K₂PO₄, 13.6 g KH₂PO₄, 2.0 g MgSO₄*7H₂O), 2.0 g citric acid monohydrate, 0.3 g ferric ammonium citrate, 3.2 g (NH₄)₂SO₄, 0.2 g yeast extract, 1.0 ml 1000× Modified Trace Metal Solution, adjusted to pH 6.8 and q.s. to H₂0, and filter sterilized) containing 1% glucose. Flasks were incubated at 30° C. until an OD₆₀₀ of 0.8 was reached, and then induced with 400 μM IPTG. Samples were taken at various times after induction and the amount of isoprene in the head space was measured as described in Example 13. Results are shown in FIG. 23H.

III. The Effect of Yeast Extract on Isoprene Production in E. coli Grown in Fed-Batch Culture

Fermentation was performed at the 14-L scale as previously described with E. coli cells containing the pTrcKudzu yIDI DXS plasmid described above. Yeast extract (Bio Springer, Montreal, Quebec, Canada) was fed at an exponential rate. The total amount of yeast extract delivered to the fermentor was varied between 70-830 g during the 40 hour fermentation. Optical density of the fermentation broth was measured at a wavelength of 550 nm. The final optical density within the fermentors was proportional to the amount of yeast extract added (FIG. 48A). The isoprene level in the off-gas from the fermentor was determined as previously described. The isoprene titer increased over the course of the fermentation (FIG. 48B). The amount of isoprene produced was linearly proportional to the amount of fed yeast extract (FIG. 48C).

IV. Production of Isoprene in 500 L Fermentation of pTrcKudzu DXS yIDI

A 500 liter fermentation of E. coli cells with a kudzu isoprene synthase, S. cerevisiae IDI, and E. coli DXS nucleic acids (E. coli BL21 (λDE3) pTrc Kudzu dxs yidi) was used to produce isoprene. The levels of isoprene varied from 50 to 300 μg/L over a time period of 15 hours. On the basis of the average isoprene concentrations, the average flow through the device and the extent of isoprene breakthrough, the amount of isoprene collected was calculated to be approximately 17 g.

V. Production of Isoprene in 500 L Fermentation of E. coli Grown in Fed-Batch Culture

### Medium Recipe (Per Liter Fermentation Medium):

K₂HPO₄ 7.5 g, MgSO₄*7H₂O 2 g, citric acid monohydrate 2 g, ferric ammonium citrate 0.3 g, yeast extract 0.5 g, 1000× Modified Trace Metal Solution 1 ml. All of the components were added together and dissolved in diH₂O. This solution was autoclaved. The pH was adjusted to 7.0 with ammonium gas (NH₃) and q.s. to volume. Glucose 10 g, thiamine*HCl 0.1 g, and antibiotic were added after sterilization and pH adjustment.

### 1000× Modified Trace Metal Solution:

Citric Acids*H₂O 40 g, MnSO₄*H₂O 30 g, NaCl 10 g, FeSO₄*7H₂O 1 g, CoCl₂*6H₂O 1 g, ZnSO₄*7H₂O 1 g, CuSO₄*5H₂O 100 mg, H₃BO₃ 100 mg, NaMoO₄*2H₂O 100 mg. Each component is dissolved one at a time in DI H₂O, pH to 3.0 with HCl/NaOH, then q.s. to volume and filter sterilized with 0.22 micron filter.

Fermentation was performed in a 500-L bioreactor with E. coli cells containing the pTrcKudzu yIDI DXS plasmid. This experiment was carried out to monitor isoprene formation from glucose and yeast extract at the desired fermentation pH 7.0 and temperature 30° C. An inoculum of E. coli strain taken from a frozen vial was prepared in soytone-yeast extract-glucose medium. After the inoculum grew to OD 0.15, measured at 550 nm, 20 ml was used to inoculate a bioreactor containing 2.5-L soytone-yeast extract-glucose medium. The 2.5-L bioreactor was grown at 30° C. to OD 1.0 and 2.0-L was transferred to the 500-L bioreactor.

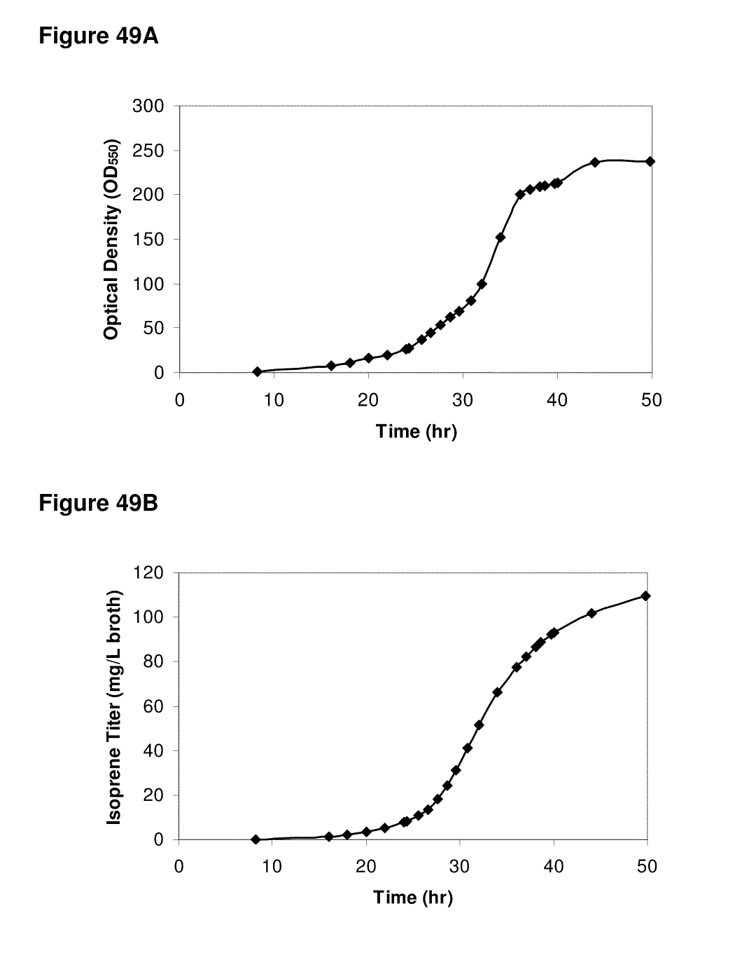

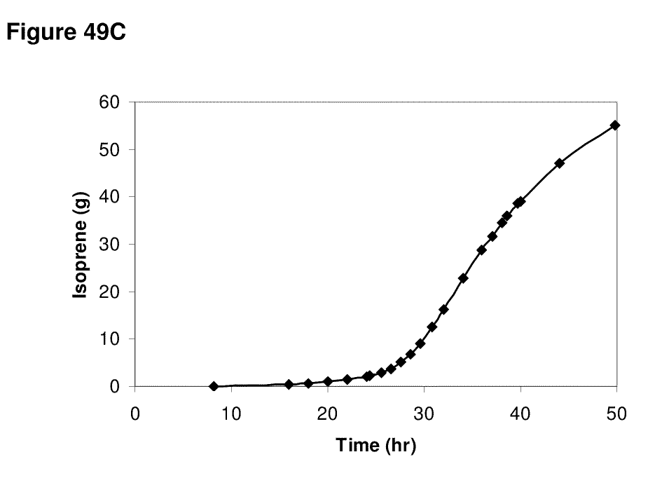

Yeast extract (Bio Springer, Montreal, Quebec, Canada) and glucose were fed at exponential rates. The total amount of glucose and yeast extract delivered to the bioreactor during the 50 hour fermentation was 181.2 kg and 17.6 kg, respectively. The optical density within the bioreactor over time is shown in FIG. 49A. The isoprene level in the off-gas from the bioreactor was determined as previously described. The isoprene titer increased over the course of the fermentation (FIG. 49B). The total amount of isoprene produced during the 50 hour fermentation was 55.1 g and the time course of production is shown in FIG. 49C.

### Example 20

### Production of Isoprene in E. coli Expressing Kudzu Isoprene Synthase and Recombinant Mevalonic Acid Pathway Genes

### I. Cloning the Lower MVA Pathway

The strategy for cloning the lower mevalonic pathway was as follows. Four genes of the mevalonic acid biosynthesis pathway; mevalonate kinase (MVK), phosphomevalonate kinase (PMK), diphosphomevalonate decarboxylase (MVD) and isopentenyl diphosphate isomerase genes were amplified by PCR from S. cerevisiae chromosomal DNA and cloned individually into the pCR BluntII TOPO plasmid (Invitrogen). In some cases, the idi gene was amplified from E. coli chromosomal DNA. The primers were designed such that an E. coli consensus RBS (AGGAGGT (SEQ ID NO:80) or AAGGAGG (SEQ ID NO:81)) was inserted at the 5′ end, 8 by upstream of the start codon and a PstI site was added at the 3′ end. The genes were then cloned one by one into the pTrcHis2B vector until the entire pathway was assembled.

Chromosomal DNA from S. cerevisiae S288C was obtained from ATCC (ATCC 204508D). The MVK gene was amplified from the chromosome of S. cerevisiae using primers MVKF (5′-AGGAGGTAAAAAAACATGTCATTACCGTTCTTAACTTCTGC, SEQ ID NO:21) and MVK-PstI-R (5′-ATGGCTGCAGGCCTATCGCAAATTAGCTTATGAAGTCCATGGTAAATTCGTG, SEQ ID NO:22) using PfuTurbo as per manufacturer's instructions. The correct sized PCR product (1370 bp) was identified by electrophoresis through a 1.2% E-gel (Invitrogen) and cloned into pZeroBLUNT TOPO. The resulting plasmid was designated pMVK1. The plasmid pMVK1 was digested with SacI and Taq1 restriction endonucleases and the fragment was gel purified and ligated into pTrcHis2B digested with SacI and BstBI. The resulting plasmid was named pTrcMVK1 (also refered to as pTrcK).

The second gene in the mevalonic acid biosynthesis pathway, PMK, was amplified by PCR using primers: PstI-PMK1 R (5′-GAATTCGCCCTTCTGCAGCTACC, SEQ ID NO:23) and BsiHKA I-PMK1 F (5′-CGACTGGTGCACCCTTAAGGAGGAAAAAAACATGTCAG, SEQ ID NO:24). The PCR reaction was performed using Pfu Turbo polymerase (Stratagene) as per manufacturer's instructions. The correct sized product (1387 bp) was digested with PstI and BsiHKI and ligated into pTrcMVK1 digested with PstI. The resulting plasmid was named pTrcKK.

The MVD and the idi genes were cloned in the same manner. PCR was carried out using the primer pairs PstI-MVD 1 R (5′-GTGCTGGAATTCGCCCTTCTGCAGC, SEQ ID NO:25) and NsiI-MVD 1 F (5′-GTAGATGCATGCAGAATTCGCCCTTAAGGAGG, SEQ ID NO:26) to amplify the MVD gene and PstI-YIDI 1 R (5′-CCTTCTGCAGGACGCGTTGTTATAGC, SEQ ID NO:27) and NsiI-YIDI 1 F (5′-CATCAATGCATCGCCCTTAGGAGGTAAAAAAAAATGAC, SEQ ID NO:28) to amplify the yIDI gene. The plasmid with the MVK, PMK, and MVD genes inserted is named pTrcKKD. In some cases the IPP isomerase gene, idi from E. coli was used. To amplify idi from E. coli chromosomal DNA, the following primer set was used: PstI-CIDI 1 R (5′-GTGTGATGGATATCTGCAGAATTCG, SEQ ID NO:29) and NsiI-CIDI 1 F (5′-CATCAATGCATCGCCCTTAGGAGGTAAAAAAACATG, SEQ ID NO:30). Template DNA was chromosomal DNA isolated by standard methods from E. coli FM5 (WO 96/35796 and WO 2004/033646, which are each hereby incorporated by reference in their entireties, particularly with respect to isolation of nucleic acids). The final plasmids were named pKKDIy for the construct encoding the yeast idi gene or pKKDIc for the construct encoding the E. coli idi gene. The plasmids were transformed into E. coli hosts BL21 for subsequent analysis. In some cases the isoprene synthase from kudzu was cloned into pKKDIy yielding plasmid pKKDIyIS.

The lower MVA pathway was also cloned into pTrc containing a kanamycin antibiotic resistance marker. The plasmid pTrcKKDIy was digested with restriction endonucleases ApaI and PstI, the 5930 by fragment was separated on a 1.2% agarose E-gel and purified using the Qiagen Gel Purification kit according to the manufacturer's instructions. The plasmid pTrcKudzuKan, described in Example 19, was digested with restriction endonucleases ApaI and PstI, and the 3338 by fragment containing the vector was purified from a 1.2% E-gel using the Qiagen Gel Purification kit. The 3338 by vector fragment and the 5930 by lower MVA pathway fragment were ligated using the Roche Quick Ligation kit. The ligation mix was transformed into E. coli TOP10 cells and tranformants were grown at 37° C. overnight with selection on LA containing kanamycin (50 μg/ml). The transformants were verified by restriction enzyme digestion and one was frozen as a stock. The plasmid was designated pTrcKanKKDIy.

II. Cloning a Kudzu Isoprene Synthase Gene into pTrcKanKKDIy

The kudzu isoprene synthase gene was amplified by PCR from pTrcKudzu, described in Example 13, using primers MCM50 5′-GATCATGCATTCGCCCTTAGGAGGTAAAAAAACATGTGTGCGACCTCTTCTCAATTTACT (SEQ ID NO:31) and MCM53 5′-CGGTCGACGGATCCCTGCAGTTAGACATACATCAGCTG (SEQ ID NO:32). The resulting PCR fragment was cloned into pCR2.1 and transformed into E. coli TOP10. This fragment contains the coding sequence for kudzu isoprene synthase and an upstream region containing a RBS from E. coli. Transformants were incubated overnight at 37° C. with selection on LA containing carbenicillin (50 μg/ml). The correct insertion of the fragment was verified by sequencing and this strain was designated MCM93.

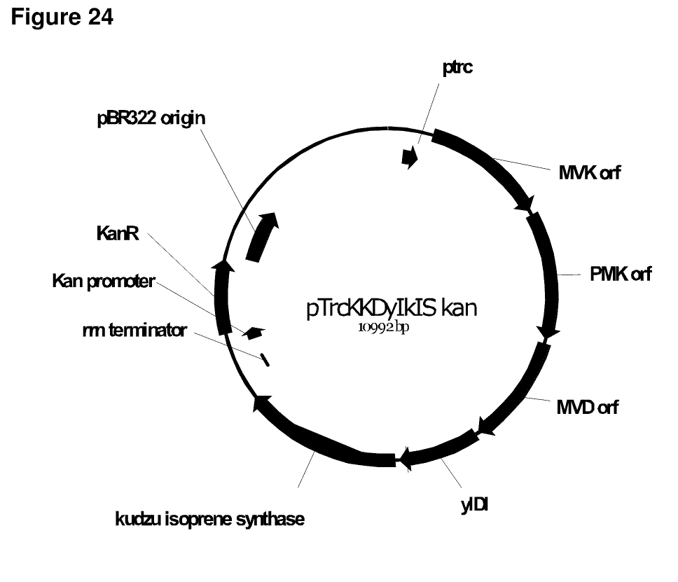

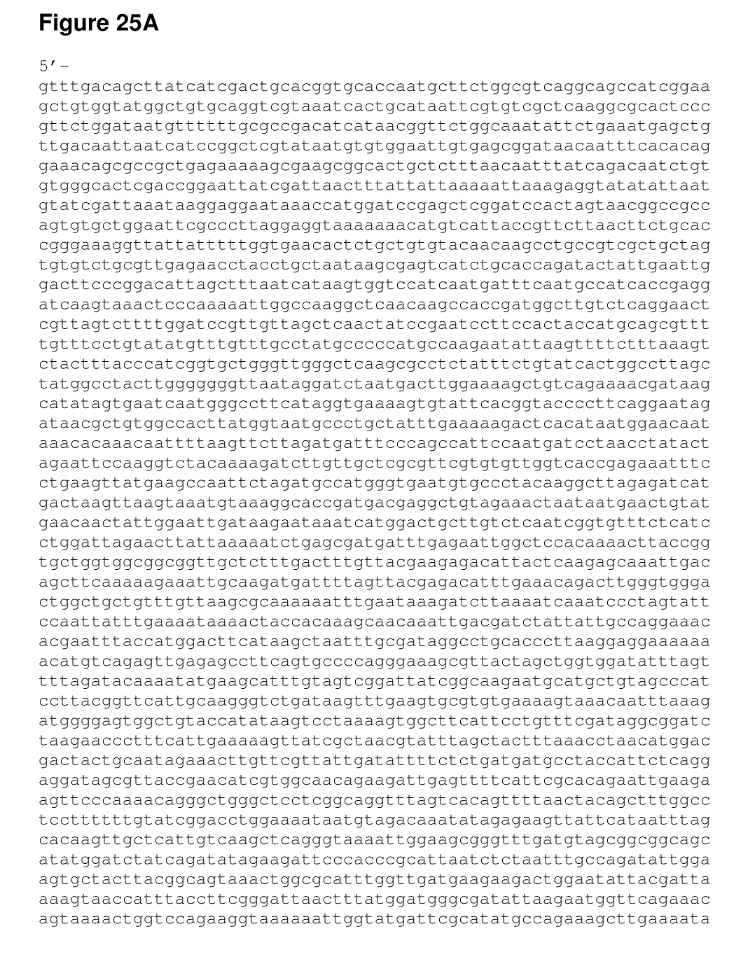

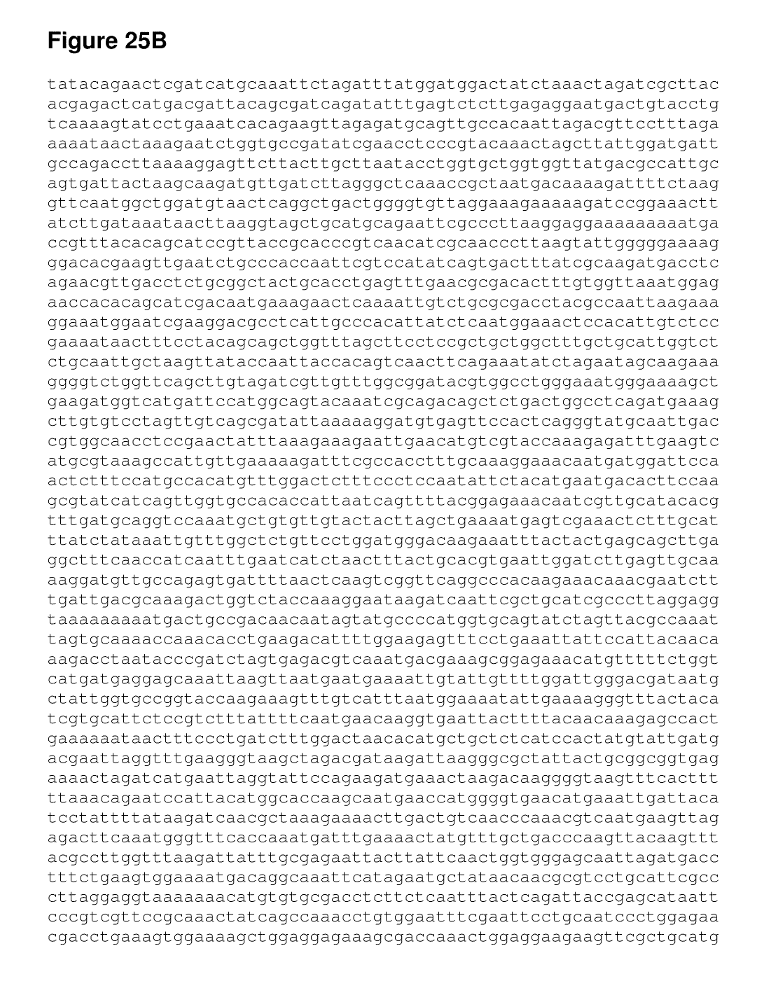

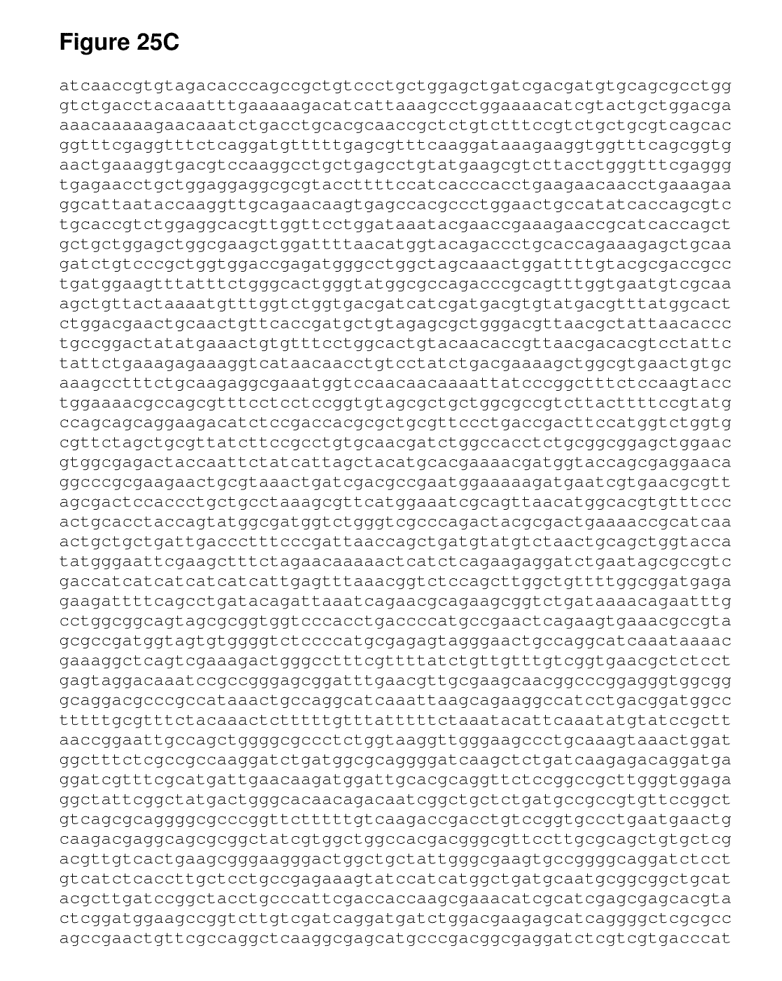

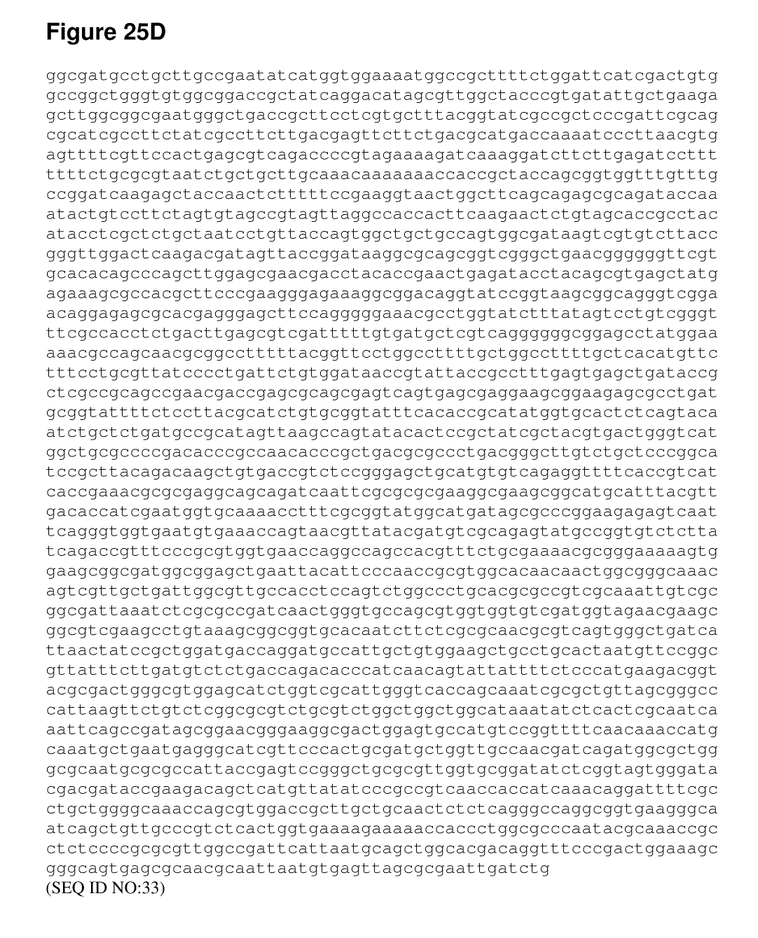

The plasmid from strain MCM93 was digested with restriction endonucleases NsiI and PstI to liberate a 1724 by insert containing the RBS and kudzu isoprene synthase. The 1724 by fragment was separated on a 1.2% agarose E-gel and purified using the Qiagen Gel Purification kit according to the manufacturer's instructions. Plasmid pTrcKanKKDIy was digested with the restriction endonuclease PstI, treated with SAP for 30 minutes at 37° C. and purified using the Qiagen PCR cleanup kit. The plasmid and kudzu isoprene synthase encoding DNA fragment were ligated using the Roche Quick Ligation kit. The ligation mix was transformed into E. coli TOP10 cells and transformants were grown overnight at 37° C. with selection on LA containing Kanamycin at 50 μg/ml. The correct transformant was verified by restriction digestion and the plasmid was designated pTrcKKDyIkISKan (FIGS. 24 and 25A-25D). This plasmid was transformed into BL21(λDE3) cells (Invitrogen).

III. Isoprene Production from Mevalonate in E. coli Expressing the Recombinant Lower Mevalonate Pathway and Isoprene Synthase from Kudzu.

Strain BL21/pTrcKKDyIkISKan was cultured in MOPS medium (Neidhardt et al., (1974) J. Bacteriology 119:736-747) adjusted to pH 7.1 and supplemented with 0.5% glucose and 0.5% mevalonic acid. A control culture was also set up using identical conditions but without the addition of 0.5% mevalonic acid. The culture was started from an overnight seed culture with a 1% inoculum and induced with 500 μM IPTG when the culture had reached an OD₆₀₀ of 0.3 to 0.5. The cultures were grown at 30° C. with shaking at 250 rpm. The production of isoprene was analyzed 3 hours after induction by using the head space assay described in Example 13. Maximum production of isoprene was 6.67×10⁻⁴ mol/L_{broth}/OD₆₀₀/hr where L_{broth} is the volume of broth and includes both the volume of the cell medium and the volume of the cells. The control culture not supplemented with mevalonic acid did not produce measurable isoprene.

### IV. Cloning the Upper MVA Pathway

The upper mevalonate biosynthetic pathway, comprising two genes encoding three enzymatic activities, was cloned from Enterococcus faecalis. The mvaE gene encodes a protein with the enzymatic activities of both acetyl-CoA acetyltransferase and 3-hydroxy-3-methylglutaryl-CoA (HMG-CoA) reductase, the first and third proteins in the pathway, and the mvaS gene encodes second enzyme in the pathway, HMG-CoA synthase. The mvaE gene was amplified from E. faecal is genomic DNA (ATCC 700802D-5) with an E. coli ribosome binding site and a spacer in front using the following primers:

CF 07-60 (+) Start of mvaE w/RBS+ATG start codon SacI

(SEQ ID NO: 34)

5′-GAGACATGAGCTCAGGAGGTAAAAAAACATGAAAACAGTAGTTATTA
TTG

CF 07-62 (−) Fuse mvaE to mvaS with RBS in between

(SEQ ID NO: 35)

5′-TTTATCAATCCCAATTGTCATGTTTTTTTACCTCCTTTATTGTTTTC
TTAAATC

The mvaS gene was amplified from E. faecalis genomic DNA (ATCC 700802D-5) with a RBS and spacer from E. coli in front using the following primers:

CF 07-61 (+) Fuse mvaE to mvaS with RBS in between

(SEQ ID NO: 36)

5′-GATTTAAGAAAACAATAAAGGAGGTAAAAAAACATGACAATTGGGAT
TGATAAA

CF 07-102 (−) End of mvaS gene BglII

(SEQ ID NO: 37)
5′-GACATGACATAGATCTTTAGTTTCGATAAGAACGAACGGT

The PCR fragments were fused together with PCR using the following primers:

CF 07-60 (+) Start of mvaE w/RBS+ATG start codon SaI

(SEQ ID NO: 34)

5′-GAGACATGAGCTCAGGAGGTAAAAAAACATGAAAACAGTAGTTATTA
TTG

CF 07-102 (−) End of mvaS gene BglII

(SEQ ID NO: 37)
5′-GACATGACATAGATCTTTAGTTTCGATAAGAACGAACGGT

The fusion PCR fragment was purified using a Qiagen kit and digested with the restriction enzymes SacI and BglII. This digested DNA fragment was gel purified using a Qiagen kit and ligated into the commercially available vector pTrcHis2A, which had been digested with SacI and BglII and gel purified.

The ligation mix was transformed into E. coli Top 10 cells and colonies were selected on LA+50 μg/ml carbenicillin plates. A total of six colonies were chosen and grown overnight in LB+50 μg/ml carbenicillin and plasmids were isolated using a Qiagen kit. The plasmids were digested with SacI and BglII to check for inserts and one correct plasmid was sequenced with the following primers:

CF 07-58 (+) Start of mvaE gene

| | |
|---|---|
| 5′-ATGAAAACAGTAGTTATTATTGATGC | (SEQ ID NO: 38) |

CF 07-59 (−) End of mvaE gene

(SEQ ID NO: 39)
5′-ATGTTATTGTTTTCTTAAATCATTTAAAATAGC

CF 07-82 (+) Start of mvaS gene

| | |
|---|---|
| 5′-ATGACAATTGGGATTGATAAAATTAG | (SEQ ID NO: 40) |

CF 07-83 (−) End of mvaS gene

| | |
|---|---|
| 5′-TTAGTTTCGATAAGAACGAACGGT | (SEQ ID NO: 41) |

CF 07-86 (+) Sequence in mvaE

| | |
|---|---|
| 5′-GAAATAGCCCCATTAGAAGTATC | (SEQ ID NO: 42) |

CF 07-87 (+) Sequence in mvaE

| | |
|---|---|
| 5′-TTGCCAATCATATGATTGAAAATC | (SEQ ID NO: 43) |

CF 07-88 (+) Sequence in mvaE

| | |
|---|---|
| 5′-GCTATGCTTCATTAGATCCTTATCG | (SEQ ID NO: 44) |

CF 07-89 (+) Sequence mvaS

| | |
|---|---|
| 5′-GAAACCTACATCCAATCTTTTGCCC | (SEQ ID NO: 45) |

The plasmid called pTrcHis2AUpperPathway#1 was correct by sequencing and was transformed into the commercially available E. coli strain BL21. Selection was done on LA+50 μg/ml carbenicillin. Two transformants were chosen and grown in LB+50 μg/ml carbenicillin until they reached an OD₆₀₀ of 1.5. Both strains were frozen in a vial at −80° C. in the presence of glycerol. Strains were designated CF 449 for pTrcHis2AUpperPathway#1 in BL21, isolate #1 and CF 450 for pTrcHis2AUpperPathway#1 in BL21, isolate #2. Both clones were found to behave identically when analyzed.

V. Cloning of UpperMVA Pathway into pCL1920

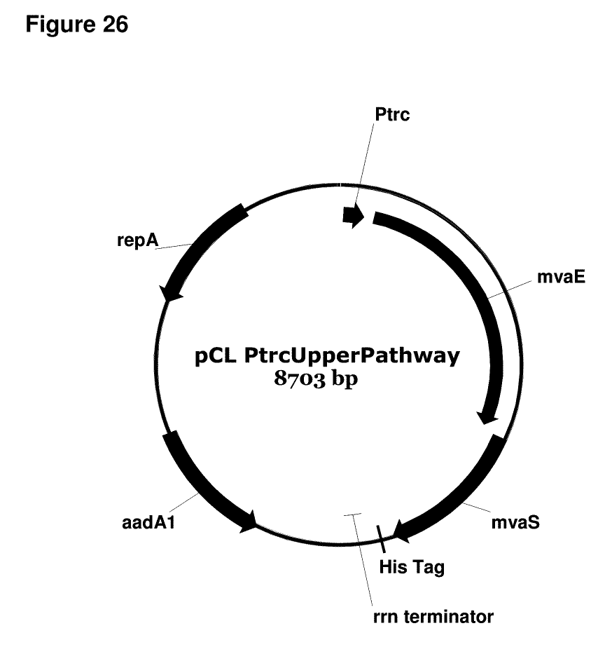

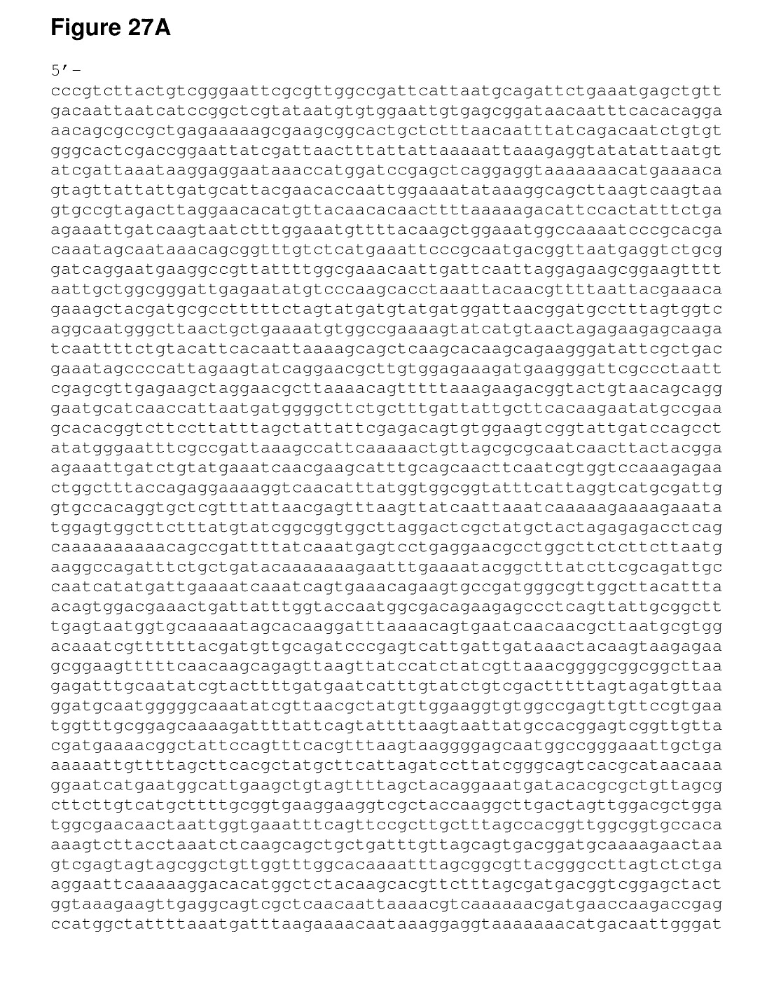

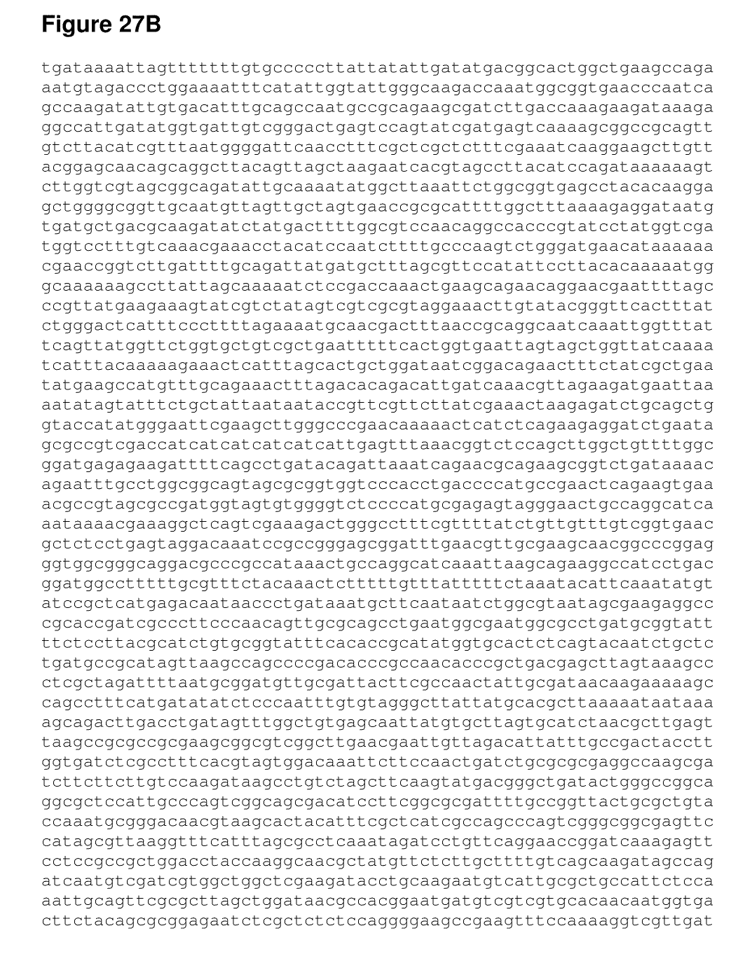

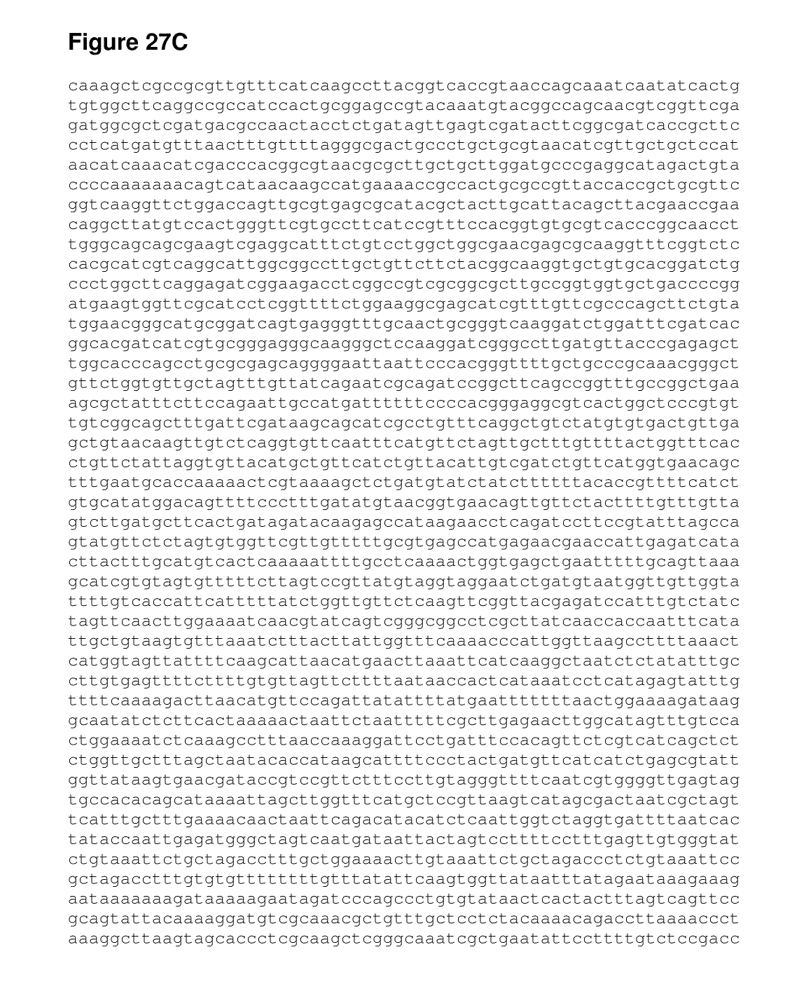

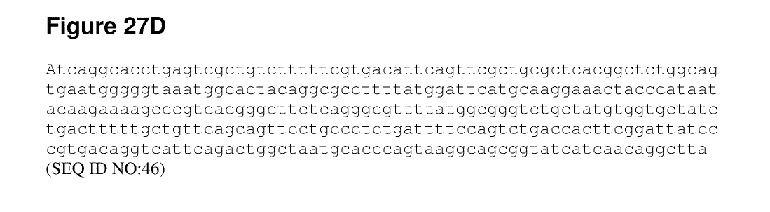

The plasmid pTrcHis2AUpperPathway was digested with the restriction endonuclease SspI to release a fragment containing pTrc-mvaE-mvaS-(His tag)-terminator. In this fragment, the his-tag was not translated. This blunt ended 4.5 kbp fragment was purified from a 1.2% E-gel using the Qiagen Gel Purification kit. A dephosphorylated, blunt ended 4.2 kbp fragment from pCL1920 was prepared by digesting the vector with the restriction endonuclease PvuII, treating with SAP and gel purifying from a 1.2% E-gel using the Qiagen Gel Purification kit. The two fragments were ligated using the Roche Quick Ligation Kit and transformed into TOP10 chemically competent cells. Transformants were selected on LA containing spectinomycin (50 μg/ml). A correct colony was identified by screening for the presence of the insert by PCR. The plasmid was designated pCL PtrcUpperPathway (FIGS. 26 and 27A-27D).

### VI. Strains Expressing the Combined Upper and Lower Mevalonic Acid Pathways

To obtain a strain with a complete mevalonic acid pathway plus kudzu isoprene synthase, plasmids pTrcKKDyIkISkan and pCLpTrcUpperPathway were both transformed into BL21(λDE3) competent cells (Invitrogen) and transformants were selected on LA containing kanamycin (50 μg/ml) and Spectinomycin (50 μg/ml). The transformants were checked by plasmid prep to ensure that both plasmids were retained in the host. The strain was designated MCM127.

VII. Production of Mevalonic Acid from Glucose in E. coli/pUpperpathway

Single colonies of the BL21/pTrcHis2A-mvaE/mvaS or FM5/p pTrcHis2A-mvaE/mvaS are inoculated into LB+carbenicillin (100 μg/ml) and are grown overnight at 37° C. with shaking at 200 rpm. These cultures were diluted into 50 ml medium in 250 ml baffled flasks to an OD₆₀₀ of 0.1. The medium was TM3+1 or 2% glucose+carbenicillin (100 ug/ml) or TM3 +1% glucose+hydrolyzed soy oil+carbenicillin (100 ug/ml) or TM3+biomass (prepared bagasse, corn stover or switchgrass). Cultures were grown at 30° C. with shaking at 200 rpm for approximately 2-3 hours until an OD₆₀₀ of 0.4 was reached. At this point the expression from the mvaE mvaS construct was induced by the addition of IPTG (400 μM). Cultures were incubated for a further 20 or 40 hours with samples taken at 2 hour intervals to 6 hour post induction and then at 24, 36 and 48 hours as needed. Sampling was done by removing 1 ml of culture, measuring the OD₆₀₀, pelleting the cells in a microfuge, removing the supernatant and analyzing it for mevalonic acid.

A 14 liter fermentation of E. coli cells with nucleic acids encoding Enterococcus faecalis AA-CoA thiolase, HMG-CoA synthase, and HMG-CoA reductase polypeptides produced 22 grams of mevalonic acid with TM3 medium and 2% glucose as the cell medium. A shake flask of these cells produced 2-4 grams of mevalonic acid per liter with LB medium and 1% glucose as the cell culture medium. The production of mevalonic acid in these strains indicated that the MVA pathway was functional in E. coli.

VIII. Production of Isoprene from E. coli BL21 Containing the Upper and Lower MVA Pathway Plus Kudzu Isoprene Synthase.

The following strains were created by transforming in various combinations of plasmids containing the upper and lower MVA pathway and the kudzu isoprene synthase gene as described above and the plasmids containing the idi, dxs, and dxr and isoprene synthase genes described in Example 19. The host cells used were chemically competent BL21(λDE3) and the transformations were done by standard methods. Transformants were selected on L agar containing kanamycin (50 μg/ml) or kanamycin plus spectinomycin (both at a concentration of 50 μg/ml). Plates were grown at 37° C. The resulting strains were designated as follows:

Grown on Kanamycin plus Spectinomycin (50 μg/ml each)
MCM127-pCL Upper MVA+pTrcKKDyIkIS (kan) in BL21(λDE3)
MCM131-pCL1920+pTrcKKDyIkIS (kan) in BL21(λDE3)
MCM125-pCL Upper MVA+pTrcHis2B (kan) in BL21(λDE3)
   Grown on Kanamycin (50 μg/ml)
MCM64-pTrcKudzu yIDI DXS (kan) in BL21(λDE3)
MCM50-pTrcKudzu (kan) in BL21(λDE3)
MCM123-pTrcKudzu yIDI DXS DXR (kan) in BL21(λDE3)

The above strains were streaked from freezer stocks to LA+appropriate antibiotic and grown overnight at 37° C. A single colony from each plate was used to inoculate shake flasks (25 ml LB+the appropriate antibiotic). The flasks were incubated at 22° C. overnight with shaking at 200 rpm. The next morning the flasks were transferred to a 37° C. incubator and grown for a further 4.5 hours with shaking at 200 rpm. The 25 ml cultures were centrifuged to pellet the cells and the cells were resuspended in 5 ml LB+the appropriate antibiotic. The cultures were then diluted into 25 ml LB+1% glucose +the appropriate antibiotic to an OD₆₀₀ of 0.1. Two flasks for each strain were set up, one set for induction with IPTG (800 μM) the second set was not induced. The cultures were incubated at 37° C. with shaking at 250 rpm. One set of the cultures were induced after 1.50 hours (immediately following sampling time point 1). At each sampling time point, the OD₆₀₀ was measured and the amount of isoprene determined as described in Example 13. Results are presented in Table 10. The amount of isoprene made is presented as the amount at the peak production for the particular strain.

TABLE 10
Production of isoprene in E. coli strains

| Strain | Isoprene (μg/liter/OD/hr |
|---|---|
| MCM50 | 23.8 |
| MCM64 | 289 |
| MCM125 | ND |
| MCM131 | Trace |
| MCM127 | 874 |

ND: not detected
Trace: peak present but not integrable.

### IX. Analysis of Mevalonic Acid

Mevalonolactone (1.0 g, 7.7 mmol) (CAS#503-48-0) was supplied from Sigma-Aldrich (WI, USA) as a syrup that was dissolved in water (7.7 mL) and was treated with potassium hydroxide (7.7 mmol) in order to generate the potassium salt of mevalonic acid. The conversion to mevalonic acid was confirmed by ¹H NMR analysis. Samples for HPLC analysis were prepared by centrifugation at 14,000 rpm for 5 minutes to remove cells, followed by the addition of a 300 μl aliquot of supernatant to 900 μl of H₂O. Perchloric acid (36 μl of a 70% solution) was then added followed by mixing and cooling on ice for 5 minutes. The samples were then centrifuged again (14,000 rpm for 5 min) and the supernatant transferred to HPLC. Mevalonic acid standards (20, 10, 5, 1 and 0.5 g/L) were prepared in the same fashion. Analysis of mevalonic acid (20 uL injection volume) was performed by HPLC using a BioRad Aminex 87-H+column (300 mm by 7.0 mm) eluted with 5 mM sulfuric acid at 0.6 mL/min with refractive index (RI) detection. Under these conditions mevalonic acid eluted as the lactone form at 18.5 minutes.

X. Production of Isoprene from E. coli BL21 Containing the Upper MVA Pathway Plus Kudzu Isoprene Synthase

A 15-L scale fermentation of E. coli expressing mevalonic acid pathway polypeptides and Kudzu isoprene synthase was used to produce isoprene from cells in fed-batch culture. This experiment demonstrates that growing cells under glucose limiting conditions resulted in the production of 2.2 g/L of isoprene.

### Medium Recipe (Per Liter Fermentation Medium):

The medium was generated using the following components per liter fermentation medium: K₂HPO₄ 7.5 g, MgSO₄*7H₂O 2 g, citric acid monohydrate 2 g, ferric ammonium citrate 0.3 g, yeast extract 0.5 g, and 1000× modified trace metal solution 1 ml. All of the components were added together and dissolved in diH₂O. This solution was autoclaved. The pH was adjusted to 7.0 with ammonium hydroxide (30%) and q.s. to volume. Glucose 10 g, thiamine*HCl 0.1 g, and antibiotics were added after sterilization and pH adjustment.

### 1000× Modified Trace Metal Solution:

The 1000× modified trace metal solution was generated using the following components: citric acids*H₂O 40 g, MnSO₄*H₂O 30 g, NaCl 10 g, FeSO₄*7H₂O 1 g, CoCl₂*6H₂O 1 g, ZnSO₄*7H₂O 1 g, CuSO₄*5H₂O 100 mg, H₃BO₃ 100 mg, and NaMoO₄*2H₂O 100 mg. Each component was dissolved one at a time in diH₂O, pH to 3.0 with HCl/NaOH, then q.s. to volume,and filter sterilized with a 0.22 micron filter.

Fermentation was performed in a 15-L bioreactor with BL21 (DE3) E. coli cells containing the pCL PtrcUpperPathway (FIG. 26) and pTrcKKDyIkIS plasmids. This experiment was carried out to monitor isoprene formation from glucose at the desired fermentation pH 7.0 and temperature 30° C. An inoculum of E. coli strain taken from a frozen vial was streaked onto an LB broth agar plate (with antibiotics) and incubated at 37° C. A single colony was inoculated into soytone-yeast extract-glucose medium. After the inoculum grew to OD 1.0 when measured at 550 nm, 500 mL was used to inoculate a 15-L bioreactor containing an initial working volume of 5 L.

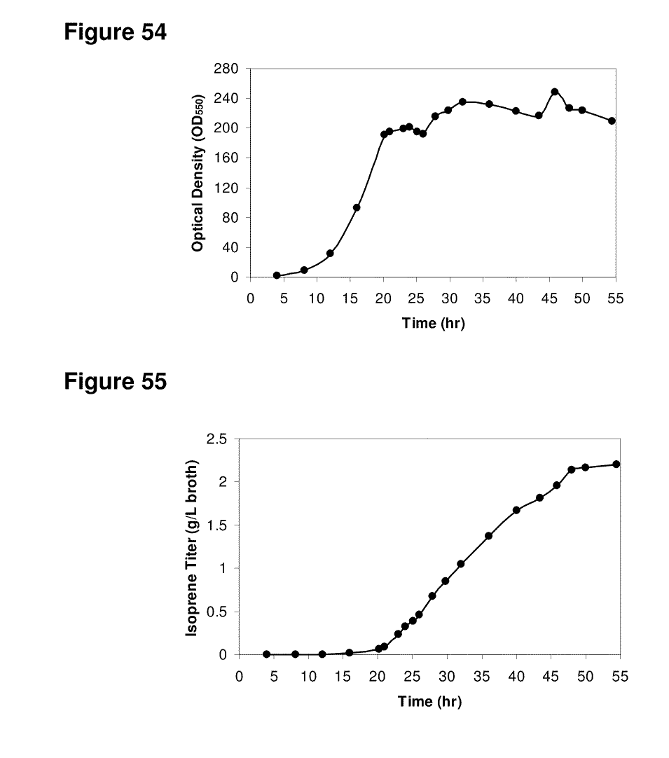

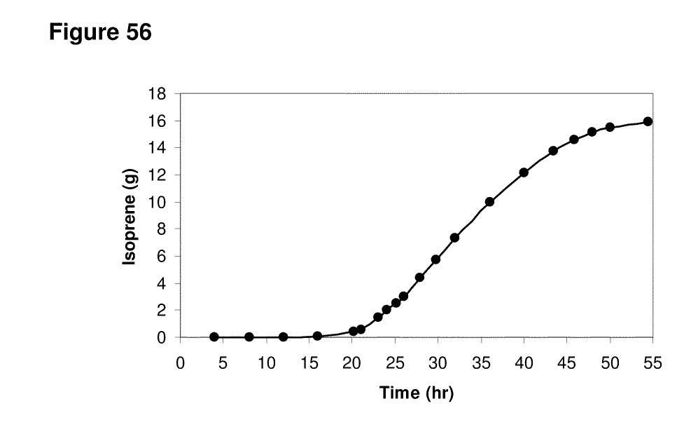

Glucose was fed at an exponential rate until cells reached the stationary phase. After this time the glucose feed was decreased to meet metabolic demands. The total amount of glucose delivered to the bioreactor during the 54 hour fermentation was 3.7 kg. Induction was achieved by adding isopropyl-beta-D-1-thiogalactopyranoside (IPTG). The IPTG concentration was brought to 25 uM when the optical density at 550 nm (OD₅₅₀) reached a value of 10. The IPTG concentration was raised to 50 uM when OD₅₅₀ reached 190. IPTG concentration was raised to 100 uM at 38 hours of fermentation. The OD₅₅₀ profile within the bioreactor over time is shown in FIG. 54. The isoprene level in the off gas from the bioreactor was determined as described herein. The isoprene titer increased over the course of the fermentation to a final value of 2.2 g/L (FIG. 55). The total amount of isoprene produced during the 54 hour fermentation was 15.9 g, and the time course of production is shown in FIG. 56.

XI. Isoprene Fermentation from E. coli Expressing Genes from the Mevalonic Acid Pathway and Grown in Fed-Batch Culture at the 15-L Scale

A 15-L scale fermentation of E. coli expressing mevalonic acid pathway polypeptides and Kudzu isoprene synthase was used to produce isoprene from cells in fed-batch culture. This experiment demonstrates that growing cells under glucose limiting conditions resulted in the production of 3.0 g/L of isoprene.

### Medium Recipe (Per Liter Fermentation Medium):

The medium was generated using the following components per liter fermentation medium: K₂HPO₄ 7.5 g, MgSO₄*7H₂O 2 g, citric acid monohydrate 2 g, ferric ammonium citrate 0.3 g, yeast extract 0.5 g, and 1000× Modified Trace Metal Solution 1 ml. All of the components were added together and dissolved in diH₂O. This solution was autoclaved. The pH was adjusted to 7.0 with ammonium hydroxide (30%) and q.s. to volume. Glucose 10 g, thiamine*HCl 0.1 g, and antibiotics were added after sterilization and pH adjustment.

### 1000× Modified Trace Metal Solution:

The 1000× modified trace metal solution was generated using the following components: citric acids*H₂O 40 g, MnSO₄*H₂O 30 g, NaCl 10 g, FeSO₄*7H₂O 1 g, CoCl₂*6H₂O 1 g, ZnSO₄*7H₂O 1 g, CuSO₄*5H₂O 100 mg, H₃BO₃ 100 mg, and NaMoO₄*2H₂O 100 mg. Each component was dissolved one at a time in diH₂O, pH to 3.0 with HCl/NaOH, then q.s. to volume, and filter sterilized with a 0.22 micron filter.

Fermentation was performed in a 15-L bioreactor with BL21 (DE3) E. coli cells containing the pCL PtrcUpperMVA and pTrc KKDyIkIS plasmids. This experiment was carried out to monitor isoprene formation from glucose at the desired fermentation pH 7.0 and temperature 30° C. An inoculum of E. coli strain taken from a frozen vial was streaked onto an LB broth agar plate (with antibiotics) and incubated at 37° C. A single colony was inoculated into tryptone-yeast extract medium. After the inoculum grew to OD 1.0, measured at 550 nm, 500 mL was used to inoculate a 15-L bioreactor containing an initial working volume of 5 L.

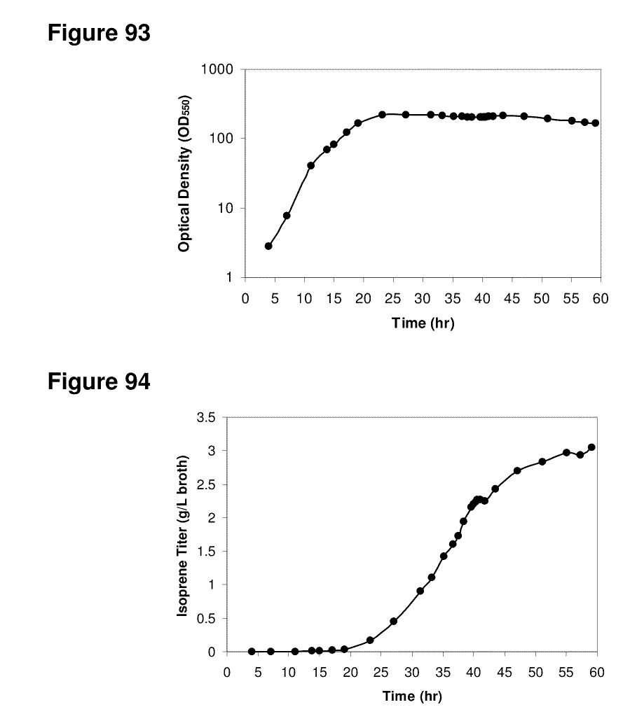

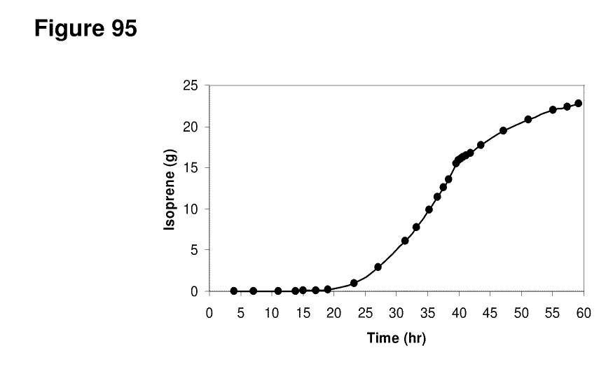

Glucose was fed at an exponential rate until cells reached the stationary phase. After this time, the glucose feed was decreased to meet metabolic demands. The total amount of glucose delivered to the bioreactor during the 59 hour fermentation was 2.2 kg. Induction was achieved by adding IPTG. The IPTG concentration was brought to 25 uM when the optical density at 550 nm (OD₅₅₀) reached a value of 10. The IPTG concentration was raised to 50 uM when OD₅₅₀ reached 190. The OD₅₅₀ profile within the bioreactor over time is shown in FIG. 93. The isoprene level in the off gas from the bioreactor was determined as described herein. The isoprene titer increased over the course of the fermentation to a final value of 3.0 g/L (FIG. 94). The total amount of isoprene produced during the 59 hour fermentation was 22.8 g, and the time course of production is shown in FIG. 95. The molar yield of utilized carbon that went into producing isoprene during fermentation was 2.2%. The weight percent yield of isoprene from glucose was 1.0%.

XII. Isoprene Fermentation from E. coli Expressing Genes from the Mevalonic Acid Pathway and Grown in Fed-Batch Culture at the 15-L Scale

A 15-L scale fermentation of E. coli expressing mevalonic acid pathway polypeptides, Pueraria lobata isoprene synthase, and Kudzu isoprene synthase was used to produce isoprene from cells in fed-batch culture. This experiment demonstrates that growing cells under glucose limiting conditions resulted in the production of 3.3 g/L of isoprene.

i) Construction of pCLPtrcUpperPathwayHGS2

The gene encoding isoprene synthase from Pueraria lobata was PCR-amplified using primers NsiI-RBS-HGS F (CTTGATGCATCCTGCATTCGCCCTTAGGAGG, SEQ ID NO:88) and pTrcR (CCAGGCAAATTCTGTTTTATCAG, SEQ ID NO:89), and pTrcKKDyIkIS as a template. The PCR product thus obtained was restriction-digested with NsiI and PstI and gel-purified. The plasmid pCL PtrcUpperPathway was restriction-digested with PstI and dephosphorylated using rAPid alkaline phosphatase (Roche) according to manufacturer's instructions.

These DNA fragments were ligated together and the ligation reaction was transformed into E. coli Top10 chemically competent cells (Invitrogen), plated on L agar containing spectinomycin (50 ug/ml) and incubated overnight at 37° C. Plasmid DNA was prepared from 6 clones using the Qiaquick Spin Mini-prep kit. The plasmid DNA was digested with restriction enzymes EcoRV and MluI to identify a clone in which the insert had the right orientation (i.e., the gene oriented in the same way as the pTrc promoter).

The resulting correct plasmid was designated pCLPtrcUpperPathwayHGS2. This plasmid was assayed using the headspace assay described herein and found to produce isoprene in E. coli Top10, thus validating the functionality of the gene. The plasmid was transformed into BL21(LDE3) containing pTrcKKDyIkIS to yield the strain BL21/pCLPtrcUpperPathwayHGS2-pTrcKKDyIkIS. This strain has an extra copy of the isoprene synthase compared to the BL21/pCL PtrcUpperMVA and pTrc KKDyIkIS strain (Example 20, part XI). This strain also had increased expression and activity of HMGS compared to the BL21/pCL PtrcUpperMVA and pTrc KKDyIkIS strain used in Example 20, part XI.

ii) Isoprene Fermentation from E. coli Expressing pCLPtrcUpperPathwayHGS2-pTrcKKDyIkIS and Grown in Fed-Batch Culture at the 15-L Scale

### Medium Recipe (Per Liter Fermentation Medium):

The medium was generated using the following components per liter fermentation medium: K₂HPO₄ 7.5 g, MgSO₄*7H₂O 2 g, citric acid monohydrate 2 g, ferric ammonium citrate 0.3 g, yeast extract 0.5 g, and 1000× modified trace metal solution 1 ml. All of the components were added together and dissolved in diH₂O. This solution was autoclaved. The pH was adjusted to 7.0 with ammonium hydroxide (30%) and q.s. to volume. Glucose 10 g, thiamine*HCl 0.1 g, and antibiotics were added after sterilization and pH adjustment.

### 1000× Modified Trace Metal Solution:

The 1000× modified trace metal solution was generated using the following components: citric acids*H₂O 40 g, MnSO₄*H₂O 30 g, NaCl 10 g, FeSO₄*7H₂O 1 g, CoCl₂*6H₂O 1 g, ZnSO₄*7H₂O 1 g, CuSO₄*5H₂O 100 mg, H₃BO₃ 100 mg, and NaMoO₄*2H₂O 100 mg. Each component is dissolved one at a time in Di H₂O, pH to 3.0 with HCl/NaOH, then q.s. to volume and filter sterilized with 0.22 micron filter.

Fermentation was performed in a 15-L bioreactor with BL21 (DE3) E. coli cells containing the pCLPtrcUpperPathwayHGS2 and pTrc KKDyIkIS plasmids. This experiment was carried out to monitor isoprene formation from glucose at the desired fermentation pH 7.0 and temperature 30° C. An inoculum of E. coli strain taken from a frozen vial was streaked onto an LB broth agar plate (with antibiotics) and incubated at 37° C. A single colony was inoculated into tryptone-yeast extract medium. After the inoculum grew to OD 1.0 measured at 550 nm, 500 mL was used to inoculate a 15-L bioreactor containing an initial working volume of 5 L.

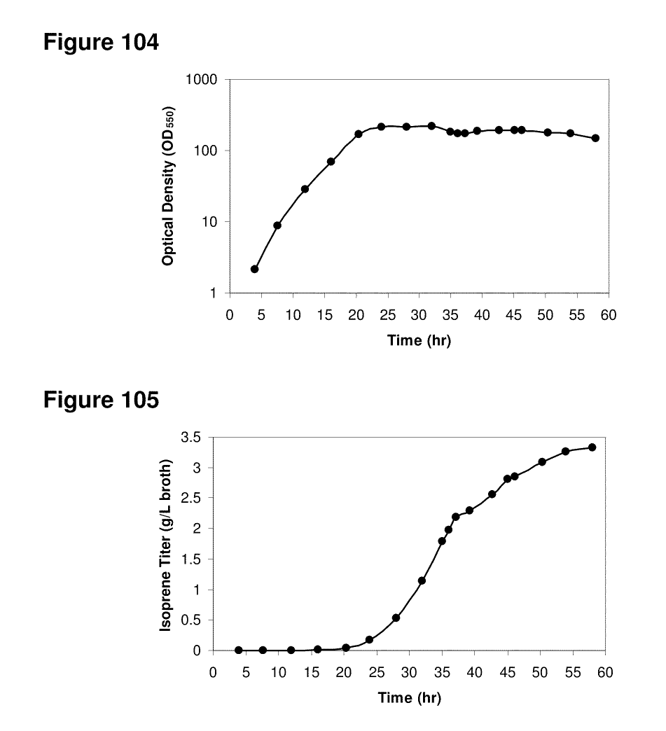

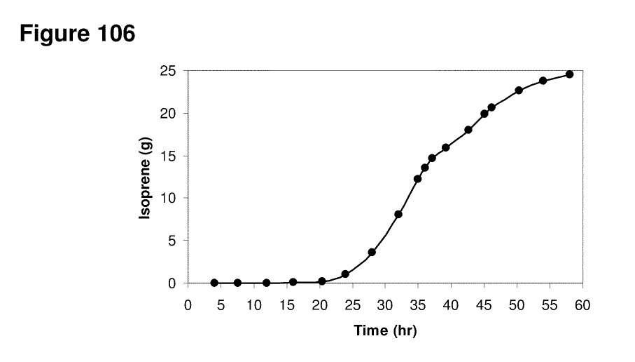

Glucose was fed at an exponential rate until cells reached the stationary phase. After this time the glucose feed was decreased to meet metabolic demands. The total amount of glucose delivered to the bioreactor during the 58 hour fermentation was 2.1 kg. Induction was achieved by adding IPTG. The IPTG concentration was brought to 25 uM when the optical density at 550 nm (OD₅₅₀) reached a value of 9. The IPTG concentration was raised to 50 uM when OD₅₅₀ reached 170. The OD₅₅₀ profile within the bioreactor over time is shown in FIG. 104. The isoprene level in the off gas from the bioreactor was determined as described herein. The isoprene titer increased over the course of the fermentation to a final value of 3.3 g/L (FIG. 105). The total amount of isoprene produced during the 58 hour fermentation was 24.5 g and the time course of production is shown in FIG. 106. The molar yield of utilized carbon that went into producing isoprene during fermentation was 2.5%. The weight percent yield of isoprene from glucose was 1.2%. Analysis showed that the activity of the isoprene synthase was increased by approximately 3-4 times that compared to BL21 expressing CL PtrcUpperMVA and pTrc KKDyIkIS plasmids (data not shown).

XIII. Chromosomal Integration of the Lower Mevalonate Pathway in E. coli.

A synthetic operon containing mevalonate kinase, mevalonate phosphate kinase, mevalonate pyrophosphate decarboxylase, and the IPP isomerase was integrated into the chromosome of E. coli. If desired, expression may be altered by integrating different promoters 5′ of the operon.

Table 11 lists primers used for this experiment.

TABLE 11
Primers

| | | |
|---|---|---|
| MCM78 | attTn7 up rev for | gcatgctcgagcggccgcTTTTAATCAAACATCCTGCCAACTC |
| | integration construct | (SEQ ID NO: 91) |
| MCM79 | attTn7 down rev for | gatcgaagggcgatcgTGTCACAGTCTGGCGAAACCG (SEQ ID |
| | integration construct | NO: 92) |
| MCM88 | attTn7 up forw for | ctgaattctgcagatatcTGTTTTTCCACTCTTCGTTCACTTT (SEQ |
| | integration construct | ID NO: 93) |
| MCM89 | attTn7 down forw for | tctagagggcccAAGAAAAATGCCCCGCTTACG (SEQ ID |
| | integration construct | NO: 94) |
| MCM104 | GI1.2 promoter - MVK | Gatcgcggccgcgcccttgacgatgccacatcctgagcaaataattcaaccactaa |
| | | ttgtgagcggataacacaaggaggaaacagctatgtcattaccgttcttaacttc |
| | | (SEQ ID NO: 95) |
| MCM105 | aspA terminator - yIDI | Gatcgggccccaagaaaaaaggcacgtcatctgacgtgccttttttatttgtagacgc |
| | | gttgttatagcattcta (SEQ ID NO: 96) |
| MCM120 | Forward of attTn7: | aaagtagccgaagatgacggtttgtcacatggagttggcaggatgtttgattaaaagc |
| | attTn7 homology, GB | AATTAACCCTCACTAAAGGGCGG (SEQ ID NO: 97) |
| | marker homology | |
| MCM127 | Rev complement of 1.2 | AGAGTGTTCACCAAAAATAATAACCTTTCCCGGTGCAgaag |
| | GI: GB marker | ttaagaacggtaatgacatagctgtttcctccttgtgttatccgctcacaattagtggttga |
| | homology(extra long), | attatttgctcaggatgtggcatcgtcaagggcTAATACGACTCACTATAG |
| | promoter, RBS, ATG | GGCTCG (SEQ ID NO: 98) |

### i) Target Vector Construction

The attTn7 site was selected for integration. Regions of homology upstream (attTn7 up) (primers MCM78 and MCM79) and downstream (attTn7 down) (primers MCM88 and MCM89) were amplified by PCR from MG1655 cells. A 50 uL reaction with 1 uL 10 uM primers, 3 uL ddH2O, 45 uL Invitrogen Platinum PCR Supermix High Fidelity, and a scraped colony of MG1655 was denatured for 2:00 at 94° C., cycled 25 times (2:00 at 94° C., 0:30 at 50° C., and 1:00 at 68° C.), extended for 7:00 at 72° C., and cooled to 4° C. This resulting DNA was cloned into pCR2.1 (Invitrogen) according to the manufacturer's instructions, resulting in plasmids MCM278 (attTn7 up) and MCM252 (attTn7 down). The 832 bp ApaI-PvuI fragment digested and gel purified from MCM252 was cloned into ApaI-PvuI digested and gel purified plasmid pR6K, creating plasmid MCM276. The 825 bp PstI-NotI fragment digested and gel purified from MCM278 was cloned into PstI-NotI digested and gel purified MCM276, creating plasmid MCM281.

### ii) Cloning of Lower Pathway and Promoter

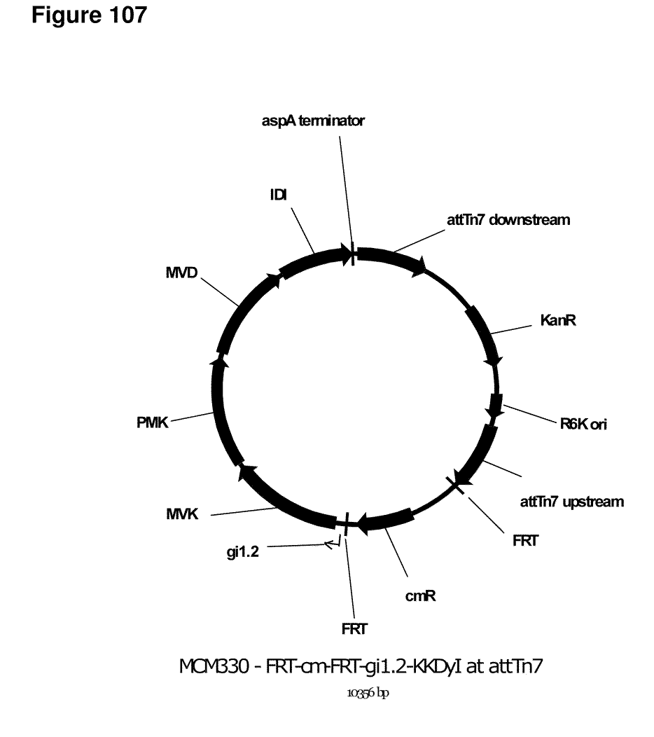

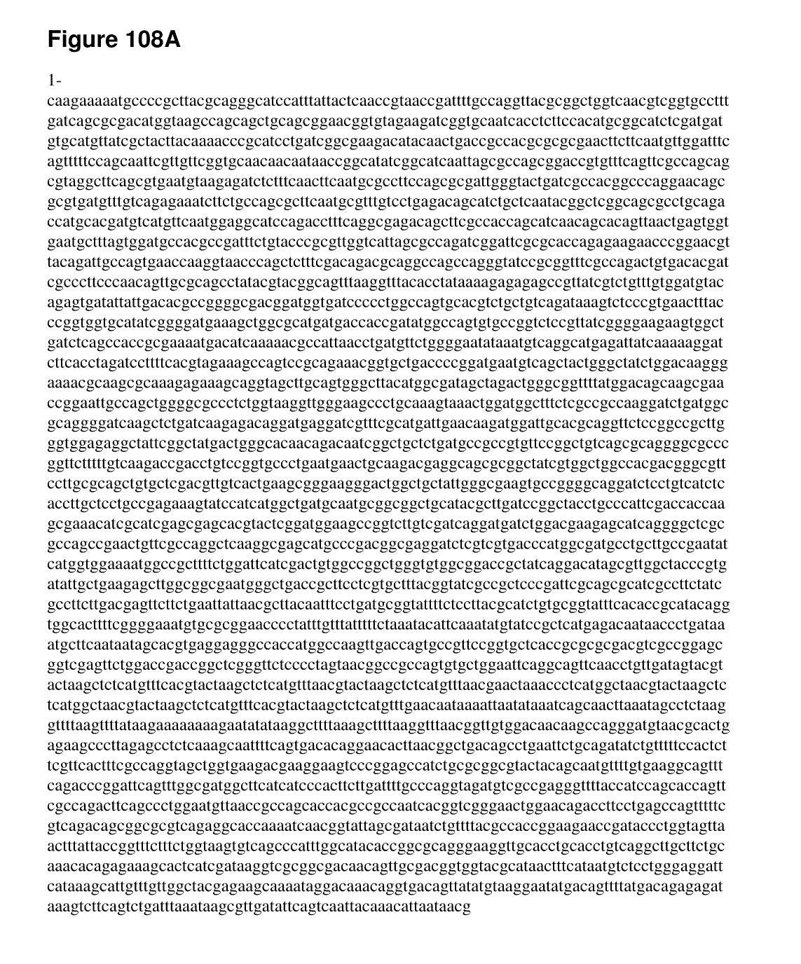

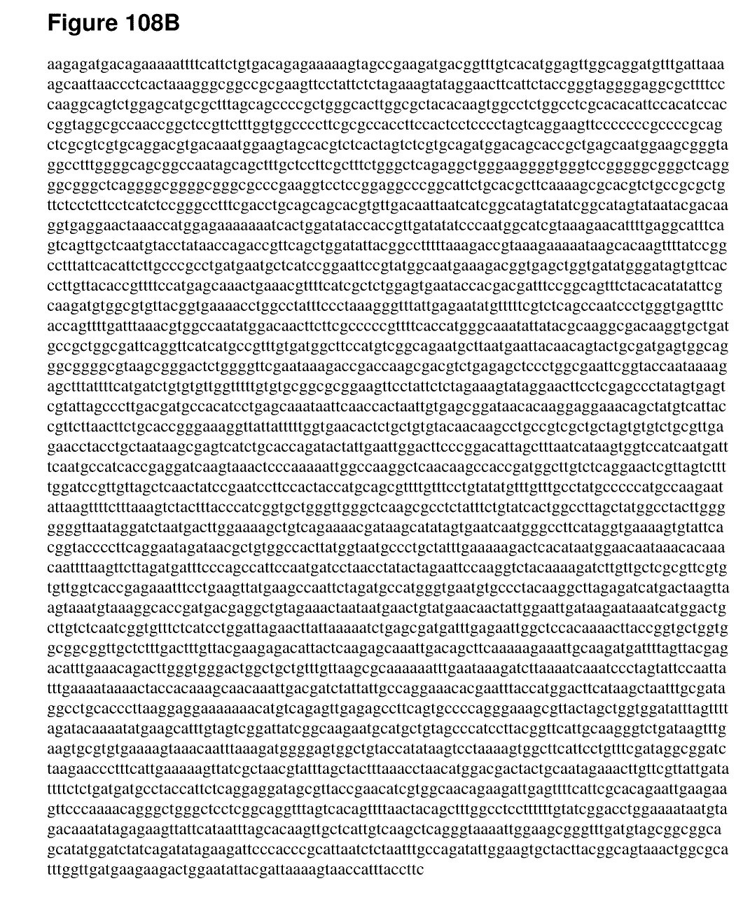

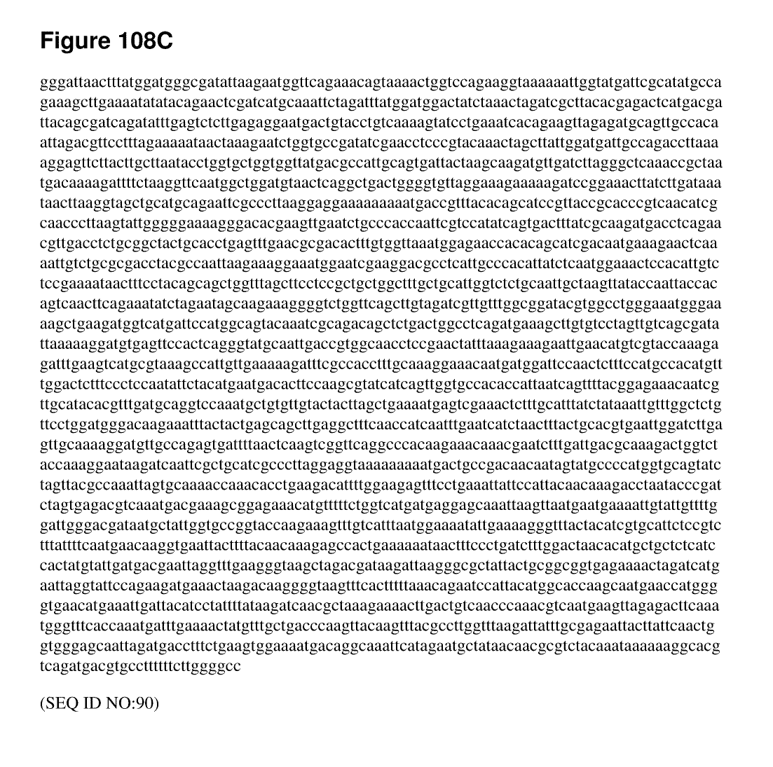

MVK-PMK-MVD-IDI genes were amplified from pTrcKKDyIkIS with primers MCM104 and MCM105 using Roche Expand Long PCR System according to the manufacturer's instructions. This product was digested with NotI and ApaI and cloned into MCM281 which had been digested with NotI and ApaI and gel purified. Primers MCM120 and MCM127 were used to amplify CMR cassette from the GeneBridges FRT-gb2-Cm-FRT template DNA using Stratagene Pfu Ultra II. A PCR program of denaturing at 95° C. for 4:00, 5 cycles of 95° C. for 0:20, 55° C. for 0:20, 72° C. for 2:00, 25 cycles of 95° C. for 0:20, 58° C. for 0:20, 72° C. for 2:00, 72° C. for 10:00, and then cooling to 4° C. was used with four 50 uL PCR reactions containing 1 uL˜10 ng/uL template, 1 uL each primer, 1.25 uL 10 mM dNTPs, 50 uL 10× buffer, 1 uL enzyme, and 39.75 uL ddH2O. Reactions were pooled, purified on a Qiagen PCR cleanup column, and used to electroporate water-washed Pir1 cells containing plasmid MCM296. Electroporation was carried out in 2 mM cuvettes at 2.5V and 200 ohms. Electroporation reactions were recovered in LB for 3 hr at 30° C. Transformant MCM330 was selected on LA with CMPS, Kan50 (FIGS. 107 and 108A-108C).

iii) Integration into E. coli Chromosome

Miniprepped DNA (Qiaquick Spin kit) from MCM330 was digested with SnaBI and used to electroporate BL21(DE3) (Novagen) or MG1655 containing GeneBridges plasmid pRedET Carb. Cells were grown at 30° C. to ˜OD1 then induced with 0.4% L-arabinose at 37° C. for 1.5 hours. These cells were washed three times in 4° C. ddH2O before electroporation with 2 uL of DNA. Integrants were selected on L agar with containing chloramphenicol (5 ug/ml) and subsequently confirmed to not grow on L agar+Kanamycin (50 ug/ml). BL21 integrant MCM331 and MG1655 integrant MCM333 were frozen.

iv) Construction of pET24D-Kudzu Encoding Kudzu Isoprene Synthase

The kudzu isoprene synthase gene was subcloned into the pET24d vector (Novagen) from the pCR2.1 vector (Invitrogen). In particular, the kudzu isoprene synthase gene was amplified from the pTrcKudzu template DNA using primers MCM50 5′-GATCATGCAT TCGCCCTTAG GAGGTAAAAA AACATGTGTG CGACCTCTTC TCAATTTACT (SEQ ID NO:99) and MCM53 5′-CGGTCGACGG ATCCCTGCAG TTAGACATAC ATCAGCTG (SEQ ID NO:100). PCR reactions were carried out using Taq DNA Polymerase (Invitrogen), and the resulting PCR product was cloned into pCR2.1-TOPO TA cloning vector (Invitrogen), and transformed into E. coli Top10 chemically competent cells (Invitrogen). Transformants were plated on L agar containing carbenicillin (50 μg/ml) and incubated overnight at 37° C. Five ml Luria Broth cultures containing carbenicillin 50 μg/ml were inoculated with single transformants and grown overnight at 37° C. Five colonies were screened for the correct insert by sequencing of plasmid DNA isolated from 1 ml of liquid culture (Luria Broth) and purified using the QIAprep Spin Mini-prep Kit (Qiagen). The resulting plasmid, designated MCM93, contains the kudzu isoprene synthase coding sequence in a pCR2.1 backbone.

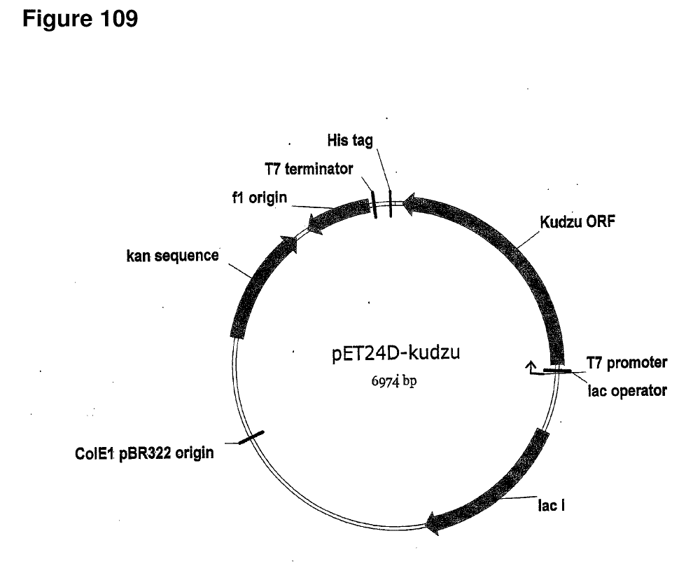

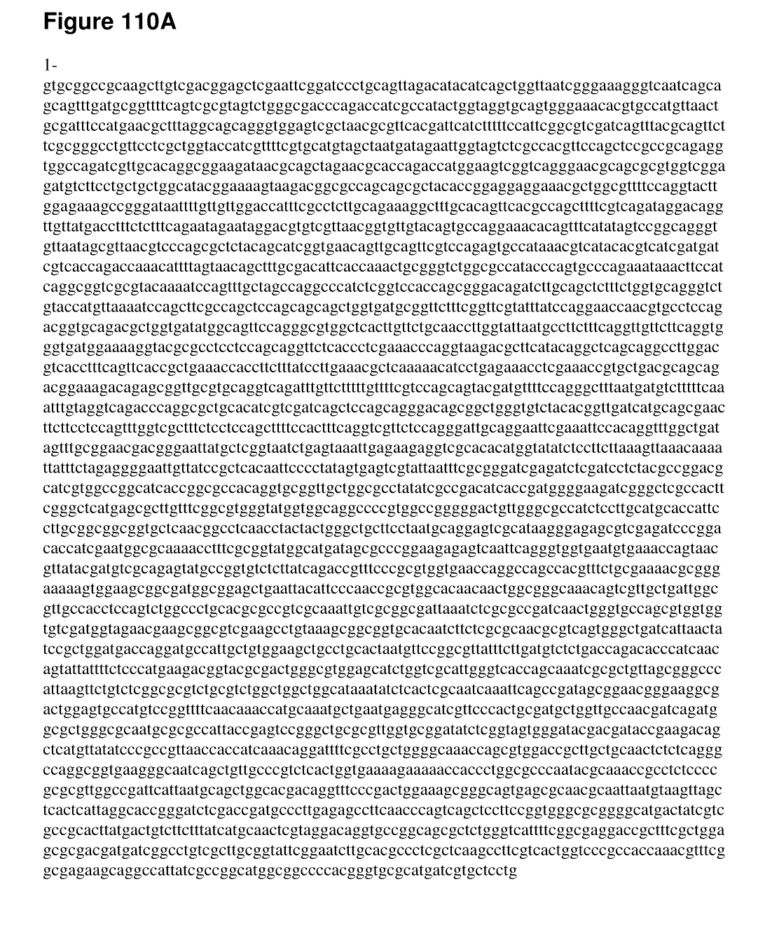

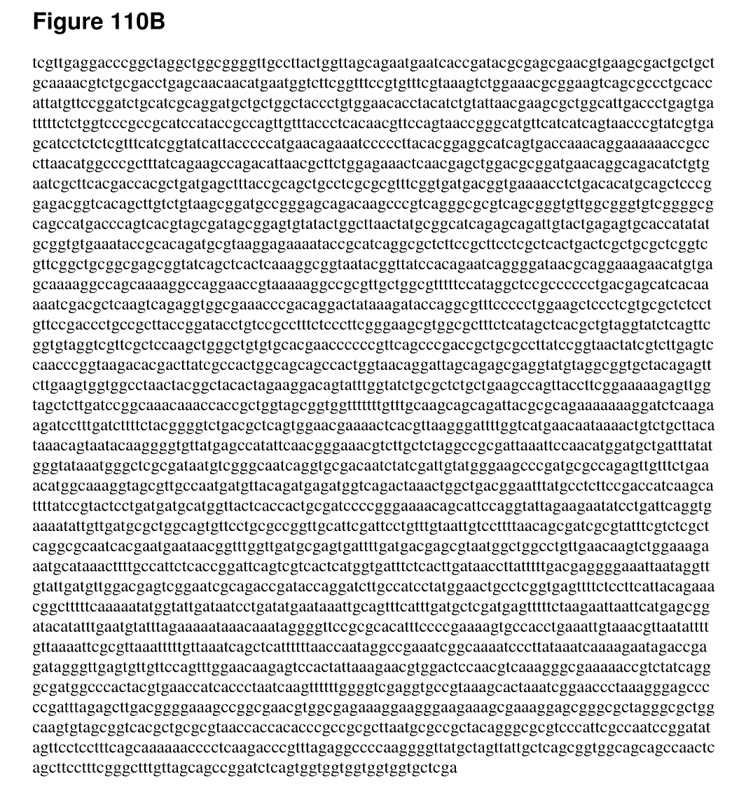

The kudzu coding sequence was removed by restriction endonuclease digestion with PciI and BamH1 (Roche) and gel purified using the QIAquick Gel Extraction kit (Qiagen). The pET24d vector DNA was digested with NcoI and BamHI (Roche), treated with shrimp alkaline phosphatase (Roche), and purified using the QIAprep Spin Mini-prep Kit (Qiagen). The kudzu isoprene synthase fragment was ligated to the NcoI/BamH1 digested pET24d using the Rapid DNA Ligation Kit (Roche) at a 5:1 fragment to vector ratio in a total volume of 20 μl. A portion of the ligation mixture (5 μl) was transformed into E. coli Top 10 chemically competent cells and plated on L agar containing kanamycin (50 μg/ml). The correct transformant was confirmed by sequencing and transformed into chemically competent BL21(λDE3)pLysS cells (Novagen). A single colony was selected after overnight growth at 37° C. on L agar containing kanamycin (50 μg/ml). A map of the resulting plasmid designated as pET24D-Kudzu is shown in FIG. 109. The sequence of pET24D-Kudzu (SEQ ID NO:101) is shown in FIGS. 110A and 110B. Isoprene synthase polypeptide activity was confirmed using a headspace assay.

### v) Production Strains

Strains MCM331 and MCM333 were cotransformed with plasmids pCLPtrcupperpathway and either pTrcKudzu or pETKudzu, resulting in the strains shown in Table 12.

TABLE 12
Production Strains

| | | | Isoprene | |
|---|---|---|---|---|
| | Integrated | Upper MVA | synthase | Production |
| Background | Lower | plasmid | plasmid | Stain |
| BL21(DE3) | MCM331 | pCLPtrcUpper | pTrcKudzu | MCM343 |
| | | Pathway | | |
| BL21(DE3) | MCM331 | pCLPtrcUpper | pET24D- | MCM335 |
| | | Pathway | Kudzu | |
| MG1655 | MCM333 | pCLPtrcUpper | pTrcKudzu | MCM345 |
| | | Pathway | | |

vi) Isoprene Fermentation from E. coli Expressing Genes from the Mevalonic Acid Pathway and Grown in Fed-Batch Culture at the 15-L Scale.

### Medium Recipe (Per Liter Fermentation Medium):

The medium was generated using the following components per liter fermentation medium: K₂HPO₄ 7.5 g, MgSO₄*7H₂O 2 g, citric acid monohydrate 2 g, ferric ammonium citrate 0.3 g, yeast extract 0.5 g, and 1000× modified trace metal solution 1 ml. All of the components were added together and dissolved in diH2O. This solution was autoclaved. The pH was adjusted to 7.0 with ammonium hydroxide (30%) and q.s. to volume. Glucose 10 g, thiamine*HCl 0.1 g, and antibiotics were added after sterilization and pH adjustment.

### 1000× Modified Trace Metal Solution:

The 1000× modified trace metal solution was generated using the following components: citric acids*H₂O 40 g, MnSO₄*H₂O 30 g, NaCl 10 g, FeSO₄*7H₂O 1 g, CoCl₂*6H₂O 1 g, ZnSO₄*7H₂O 1 g, CuSO₄*5H₂O 100 mg, H₃BO₃ 100 mg, and NaMoO₄*2H₂O 100 mg. Each component is dissolved one at a time in Di H₂O, pH to 3.0 with HCl/NaOH, then q.s. to volume and filter sterilized with a 0.22 micron filter.

Fermentation was performed in a 15-L bioreactor with BL21 (DE3) E. coli cells containing the gi1.2 integrated lower MVA pathway described above and the pCL PtrcUpperMVA and pTrcKudzu plasmids. This experiment was carried out to monitor isoprene formation from glucose at the desired fermentation pH 7.0 and temperature 30° C. An inoculum of E. coli strain taken from a frozen vial was streaked onto an LB broth agar plate (with antibiotics) and incubated at 37° C. A single colony was inoculated into tryptone-yeast extract medium. After the inoculum grew to OD 1.0, measured at 550 nm, 500 mL was used to inoculate a 15-L bioreactor containing an initial working volume of 5 L.

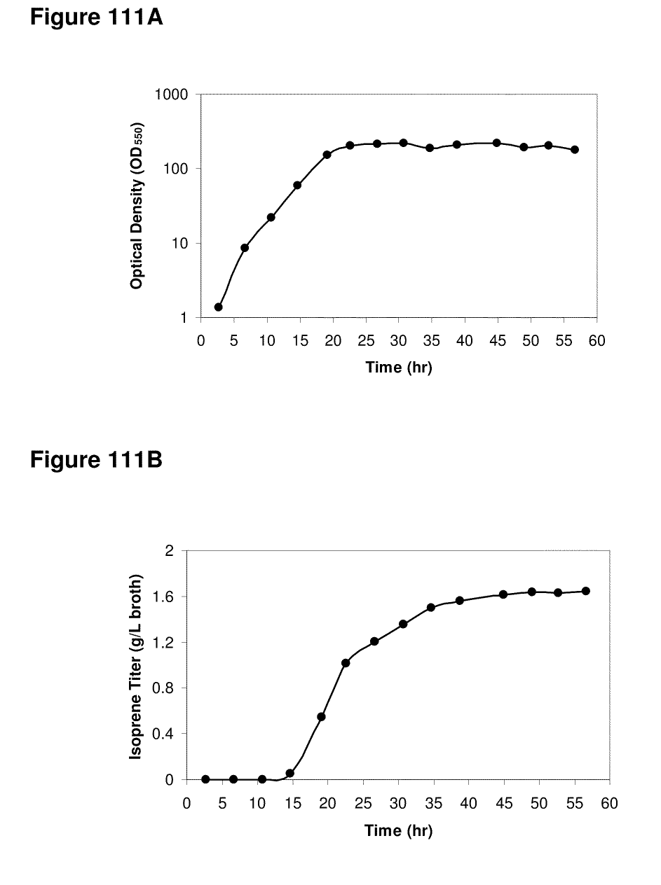

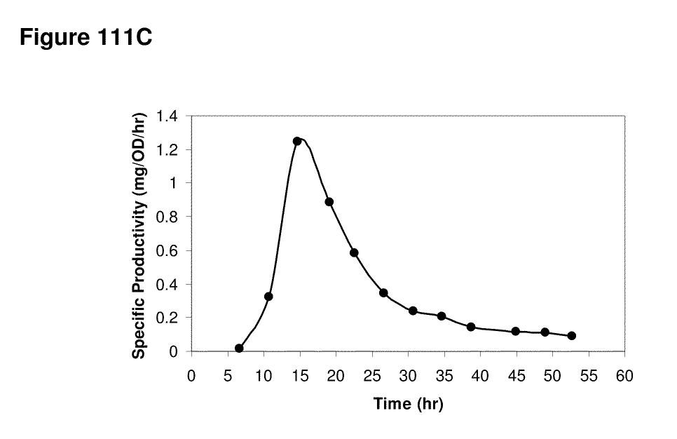

Glucose was fed at an exponential rate until cells reached the stationary phase. After this time, the glucose feed was decreased to meet metabolic demands. The total amount of glucose delivered to the bioreactor during the 57 hour fermentation was 3.9 kg. Induction was achieved by adding IPTG. The IPTG concentration was brought to 100 uM when the carbon dioxide evolution rate reached 100 mmol/L/hr. The OD₅₅₀ profile within the bioreactor over time is shown in FIG. 111A. The isoprene level in the off gas from the bioreactor was determined as described herein. The isoprene titer increased over the course of the fermentation to a final value of 1.6 g/L (FIG. 111B). The specific productivity of isoprene over the course of the fermentation is shown in FIG. 111C and peaked at 1.2 mg/OD/hr. The total amount of isoprene produced during the 57 hour fermentation was 16.2 g. The molar yield of utilized carbon that went into producing isoprene during fermentation was 0.9%. The weight percent yield of isoprene from glucose was 0.4%.

### Example 21

### Construction of the Upper and Lower MVA Pathway for Integration into Bacillus subtilis

I. Construction of the Upper MVA Pathway in Bacillus subtilis

The upper pathway from Enterococcus faecalis is integrated into B. subtilis under control of the aprE promoter. The upper pathway consists of two genes; mvaE, which encodes for AACT and HMGR, and mvaS, which encodes for HMGS. The two genes are fused together with a stop codon in between, an RBS site in front of mvaS, and are under the control of the aprE promoter. A terminator is situated after the mvaE gene. The chloramphenicol resistance marker is cloned after the mvaE gene and the construct is integrated at the aprE locus by double cross over using flanking regions of homology.

Four DNA fragments are amplified by PCR such that they contain overhangs that will allowed them to be fused together by a PCR reaction. PCR amplifications are carried out using Herculase polymerase according to manufacturer's instructions.
1. PaprE
CF 07-134 (+) Start of aprE promoter PstI

| | |
|---|---|
| 5′-GACATCTGCAGCTCCATTTTCTTCTGC | (SEQ ID NO: 82) |

CF 07-94 (−) Fuse PaprE to mvaE

(SEQ ID NO: 83)

5′-CAATAATAACTACTGTTTTCACTCTTTACCCTCTCCTTTTAA

Template: Bacillus subtilis chromosomal DNA
2. mvaE
CF 07-93 (+) fuse mvaE to the aprE promoter (GTG start codon)

(SEQ ID NO: 84)

5′-TTAAAAGGAGAGGGTAAAGAGTGAAAACAGTAGTTATTATTG

CF 07-62 (−) Fuse mvaE to mvaS with RBS in between

(SEQ ID NO: 35)

5′-TTTATCAATCCCAATTGTCATGTTTTTTTACCTCCTTTATTGTTTTC
TTAAATC

Template: Enterococcus faecalis chromosomal DNA (from ATCC)
3. mvaS
CF 07-61 (+) Fuse mvaE to mvaS with RBS in between

(SEQ ID NO: 36)

5′-GATTTAAGAAAACAATAAAGGAGGTAAAAAAACATGACAATTGGGAT
TGATAAA

CF 07-124 (−) Fuse the end of mvaS to the terminator

(SEQ ID NO: 85)

5′-CGGGGCCAAGGCCGGTTTTTTTTAGTTTCGATAAGAACGAACGGT

Template: Enterococcus faecalis chromosomal DNA
4. B. amyliquefaciens alkaline serine protease terminator
CF 07-123 (+) Fuse the end of mvaS to the terminator

(SEQ ID NO: 86)

5′-ACCGTTCGTTCTTATCGAAACTAAAAAAAACCGGCCTTGGCCCCG

CF 07-46 (−) End of B. amyliquefaciens Terminator BamHI

(SEQ ID NO: 63)

5′-GACATGACGGATCCGATTACGAATGCCGTCTC

Template: Bacillus amyliquefaciens chromosomal DNA

### PCR Fusion Reactions

5. Fuse mvaE to mvaS
CF 07-93 (+) fuse mvaE to the aprE promoter (GTG start codon)

(SEQ ID NO: 84)

5′-TTAAAAGGAGAGGGTAAAGAGTGAAAACAGTAGTTATTATTG

CF 07-124 (−) Fuse the end of mvaS to the terminator

(SEQ ID NO: 85)

5′-CGGGGCCAAGGCCGGTTTTTTTTAGTTTCGATAAGAACGAACGGT

Template: #2 and 3 from above
6. Fuse mvaE-mvaS to aprE promoter
CF 07-134 (+) Start of aprE promoter PstI

| | |
|---|---|
| 5′-GACATCTGCAGCTCCATTTTCTTCTGC | (SEQ ID NO: 82) |

CF 07-124 (−) Fuse the end of mvaS to the terminator

(SEQ ID NO: 85)

5′-CGGGGCCAAGGCCGGTTTTTTTTAGTTTCGATAAGAACGAACGGT

Template #1 and #4 from above
7. Fuse PaprE-mvaE-mvaS to terminator
CF 07-134 (+) Start of aprE promoter PstI

| | |
|---|---|
| 5′-GACATCTGCAGCTCCATTTTCTTCTGC | (SEQ ID NO: 82) |

CF 07-46 (−) End of B. amyliquefaciens terminator BamHI

(SEQ ID NO: 63)

5′-GACATGACGGATCCGATTACGAATGCCGTCTC

Template: #4 and #6

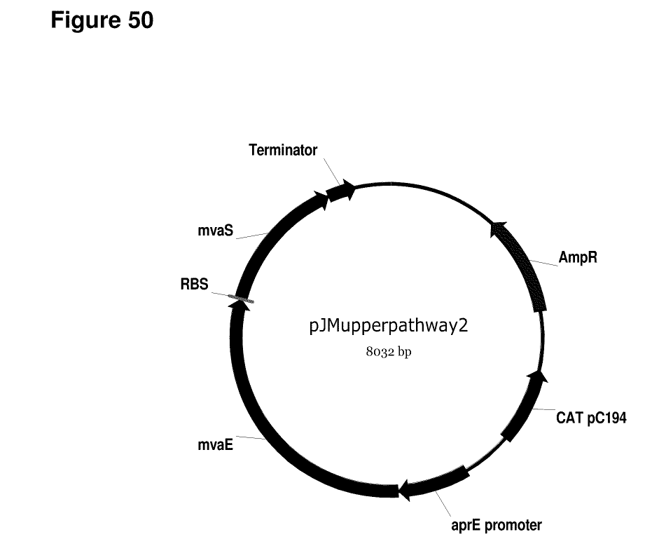

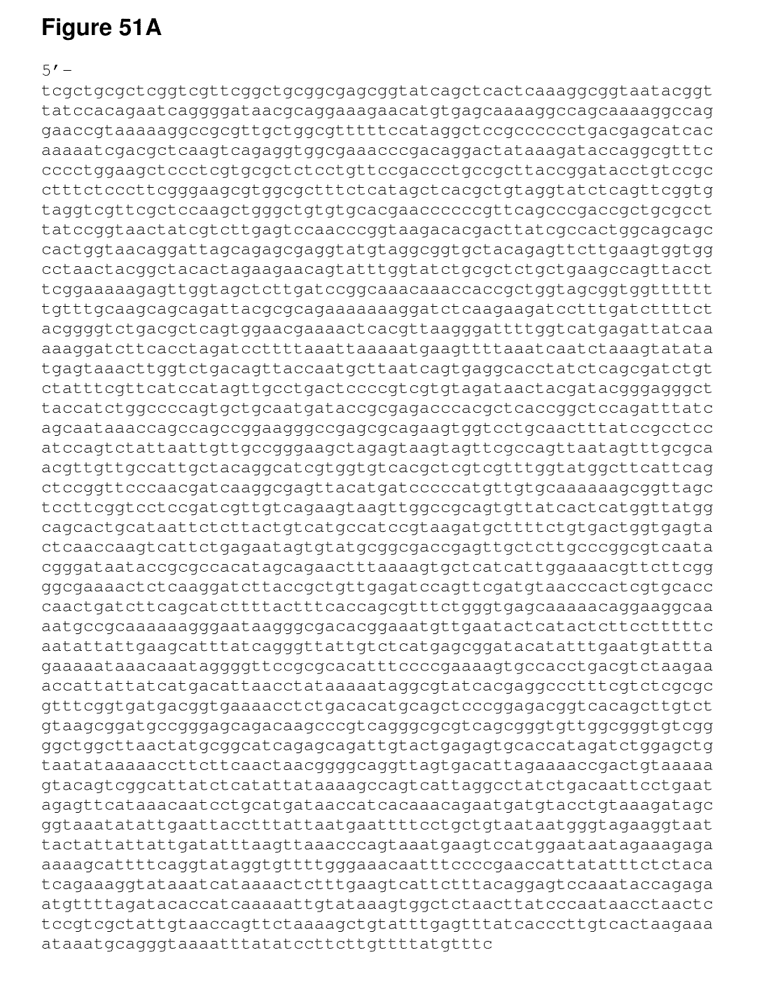

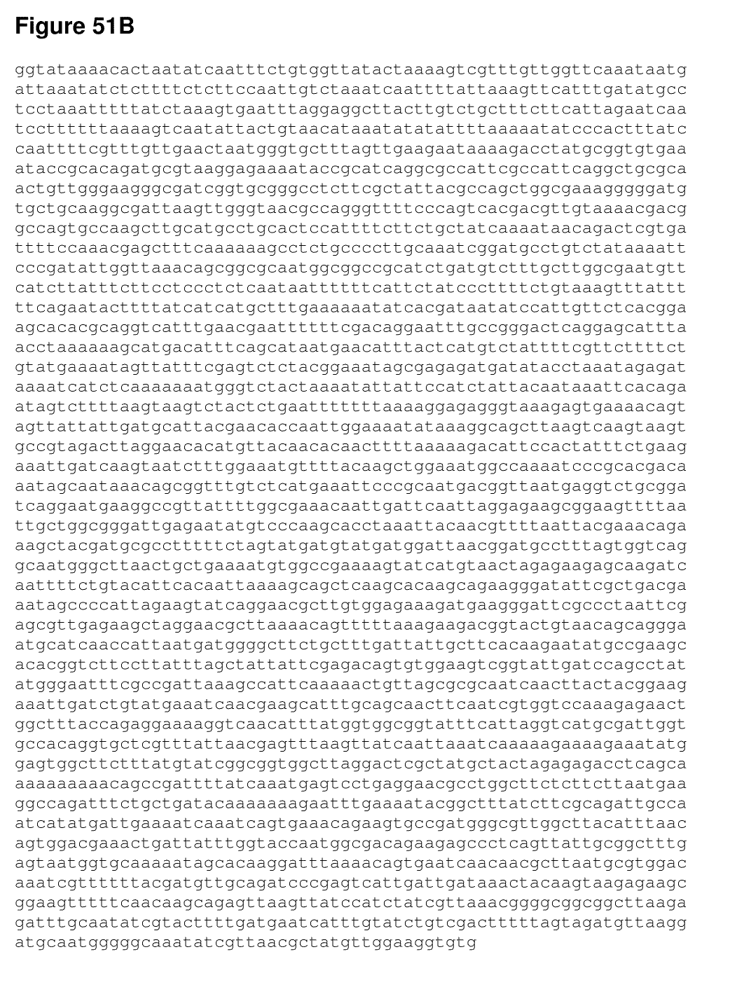

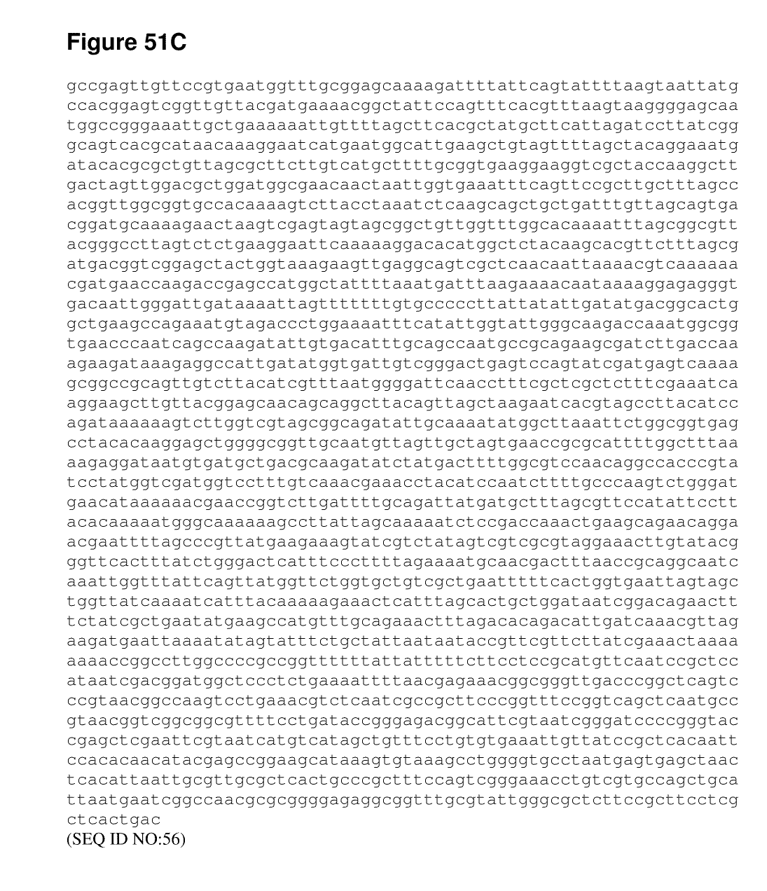

The product is digested with restriction endonucleases PstI/BamHI and ligated to pJM102 (Perego, M. 1993. Integrational vectors for genetic manipulation in Bacillus subtilis, p. 615-624. In A. L. Sonenshein, J. A. Hoch, and R. Losick (ed.), Bacillus subtilis and other gram-positive bacteria: biochemistry, physiology, and molecular genetics. American Society for Microbiology, Washington, D.C.) which is digested with PstI/BamHI. The ligation is transformed into E. coli TOP 10 chemically competent cells and transformants are selected on LA containing carbenicillin (50 μg/ml). The correct plasmid is identified by sequencing and is designated pJMUpperpathway2 (FIGS. 50 and 51A-51C). Purified plasmid DNA is transformed into Bacillus subtilis aprEnprE Pxyl-comK and transformants are selected on L agar containing chloramphenicol (5 μg/ml). A correct colony is selected and is plated sequentially on L agar containing chloramphenicol 10, 15 and 25 μg/ml to amplify the number of copies of the cassette containing the upper pathway.

The resulting strain is tested for mevalonic acid production by growing in LB containing 1% glucose and 1%. Cultures are analyzed by GC for the production of mevalonic acid.

This strain is used subsequently as a host for the integration of the lower mevalonic acid pathway.

The following primers are used to sequence the various constructs above.

Sequencing primers:
CF 07-134 (+) Start of aprE promoter PstI

| | |
|---|---|
| 5′-GACATCTGCAGCTCCATTTTCTTCTGC | (SEQ ID NO: 82) |

CF 07-58 (+) Start of mvaE gene

| | |
|---|---|
| 5′-ATGAAAACAGTAGTTATTATTGATGC | (SEQ ID NO: 38) |

CF 07-59 (−) End of mvaE gene

(SEQ ID NO: 39)

5′-ATGTTATTGTTTTCTTAAATCATTTAAAATAGC

CF 07-82 (+) Start of mvaS gene

| | |
|---|---|
| 5′-ATGACAATTGGGATTGATAAAATTAG | (SEQ ID NO: 40) |

CF 07-83 (−) End of mvaS gene

| | |
|---|---|
| 5′-TTAGTTTCGATAAGAACGAACGGT | (SEQ ID NO: 41) |

CF 07-86 (+) Sequence in mvaE

| | |
|---|---|
| 5′-GAAATAGCCCCATTAGAAGTATC | (SEQ ID NO: 42) |

CF 07-87 (+) Sequence in mvaE

| | |
|---|---|
| 5′-TTGCCAATCATATGATTGAAAATC | (SEQ ID NO: 43) |

CF 07-88 (+) Sequence in mvaE

| | |
|---|---|
| 5′-GCTATGCTTCATTAGATCCTTATCG | (SEQ ID NO: 44) |

CF 07-89 (+) Sequence mvaS

| | |
|---|---|
| 5′-GAAACCTACATCCAATCTTTTGCCC | (SEQ ID NO: 45) |

Transformants are selected on LA containing chloramphenicol at a concentration of 5 μg/ml. One colony is confirmed to have the correct integration by sequencing and is plated on LA containing increasing concentrations of chloramphenicol over several days, to a final level of 25 μg/ml. This results in amplification of the cassette containing the genes of interest. The resulting strain is designated CF 455: pJMupperpathway#1 X Bacillus subtilis aprEnprE Pxyl comK (amplified to grow on LA containing chloramphenicol 25 μg/ml).

II. Construction of the Lower MVA Pathway in Bacillus subtilis

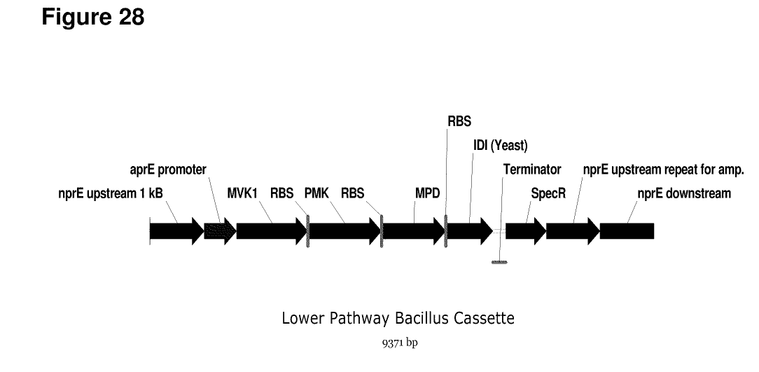

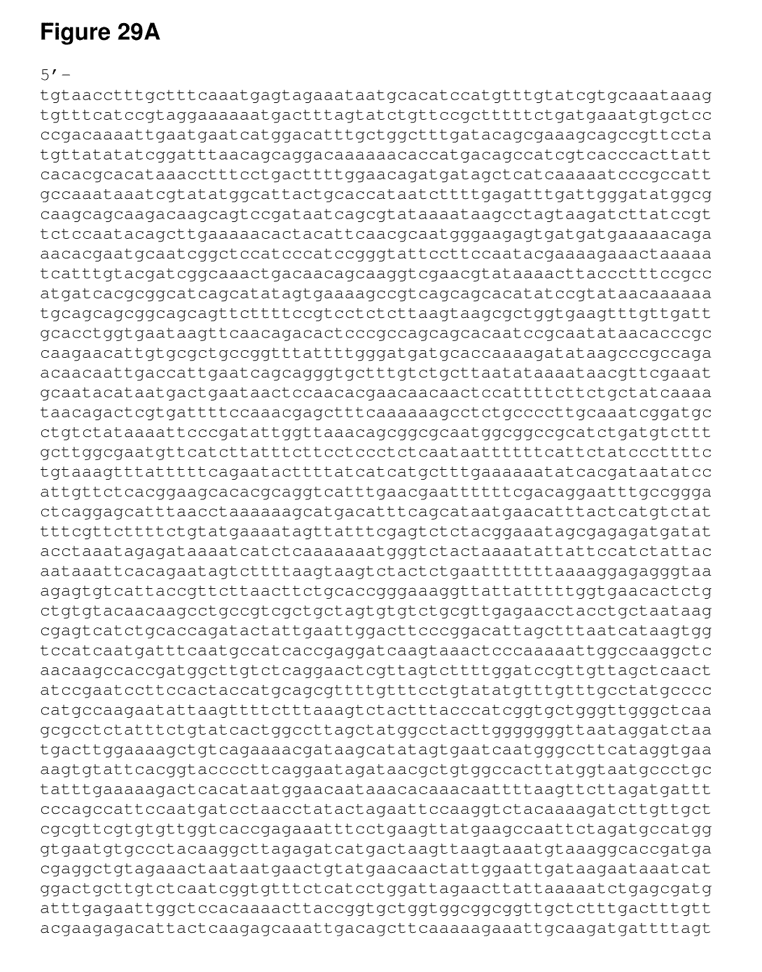

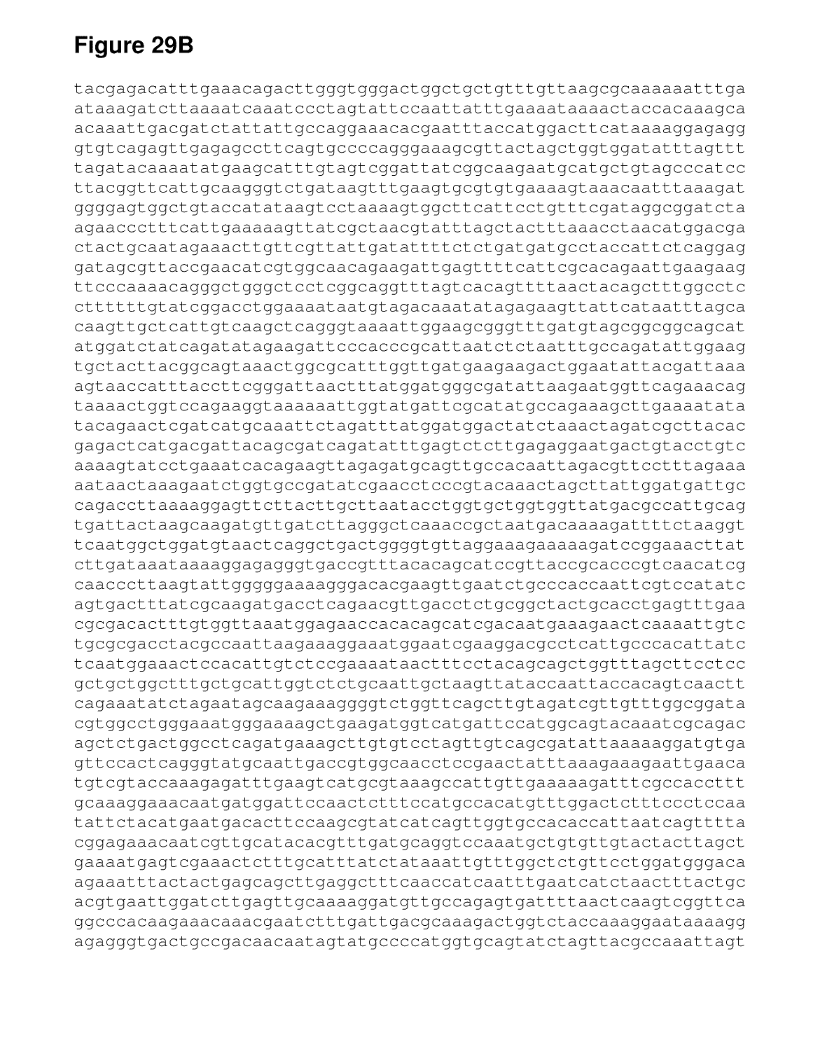

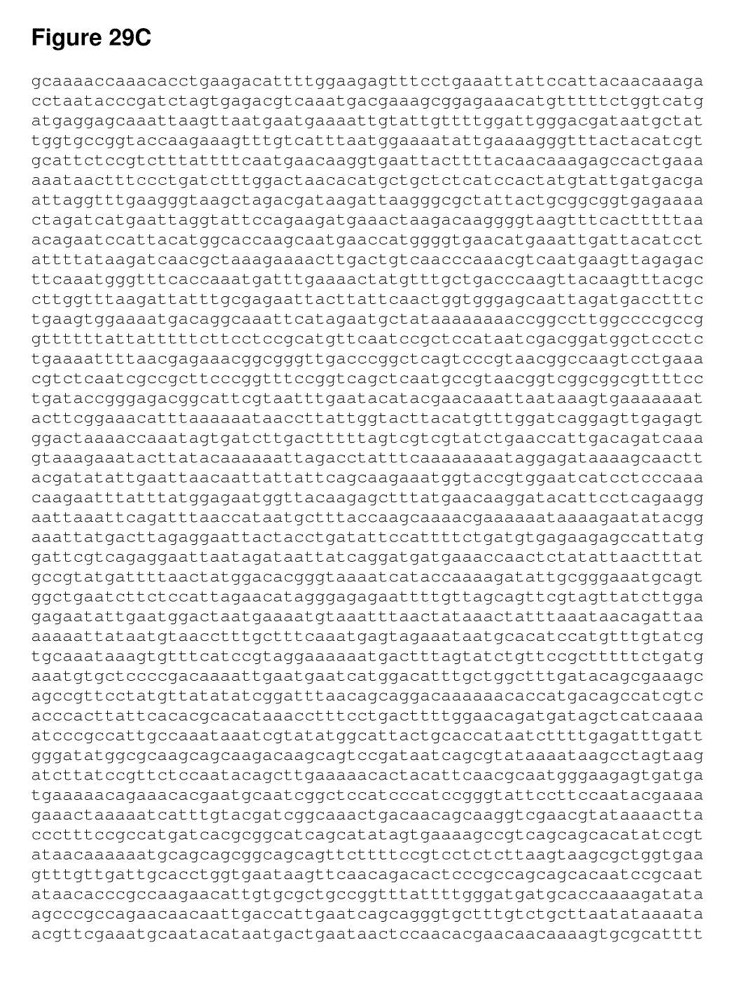

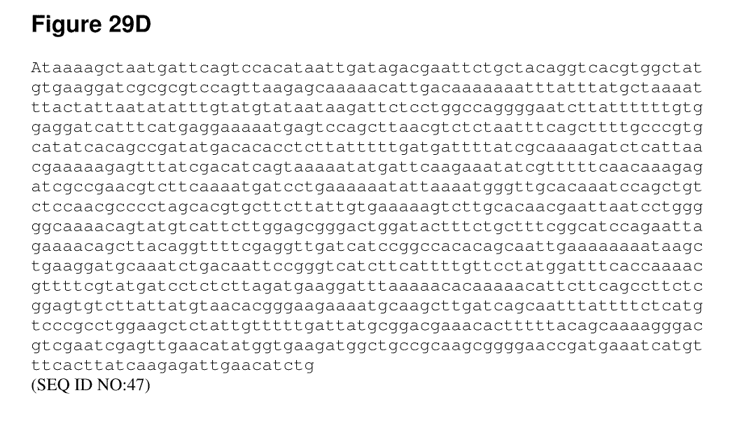

The lower MVA pathway, consisting of the genes mvk1, pmk, mpd and idi are combined in a cassette consisting of flanking DNA regions from the nprE region of the B. subtilis chromosome (site of integration), the aprE promoter, and the spectinomycin resistance marker (see FIGS. 28 and 29A-29D). This cassette is synthesized by DNA2.0 and is integrated into the chromosome of B. subtilis containing the upper MVA pathway integrated at the aprE locus. The kudzu isoprene synthase gene is expressed from the replicating plasmid described in Example 16 and is transformed into the strain with both upper and lower pathways integrated.

### Example 22

### The De-Coupling of Growth and Production of Isoprene in E. coli Expressing Genes from the Mevalonic Acid Pathway and Fermented in a Fed-Batch Culture

This example illustrates the de-coupling of cell growth from mevalonic acid and isoprene production.

### I. Fermentation Conditions

### Medium Recipe (per Liter Fermentation Medium):

The medium was generated using the following components per liter fermentation medium: K₂HPO₄ 7.5 g, MgSO₄*7H₂O 2 g, citric acid monohydrate 2 g, ferric ammonium citrate 0.3 g, yeast extract 0.5 g, and 1000× modified trace metal solution 1 ml. All of the components were added together and dissolved in diH2O. This solution was autoclaved. The pH was adjusted to 7.0 with ammonium hydroxide (30%) and q.s. to volume. Glucose 10 g, thiamine*HCl 0.1 g, and antibiotics were added after sterilization and pH adjustment.

### 1000× Modified Trace Metal Solution:

The 1000× modified trace metal solution was generated using the following components: citric acids*H₂O 40 g, MnSO₄*H₂O 30 g, NaCl 10 g, FeSO₄*7H₂O 1 g, CoCl2*6H₂O 1 g, ZnSO₄*7H₂O 1 g, CuSO₄*5H₂O 100 mg, H₃BO₃ 100 mg, and NaMoO₄*2H₂O 100 mg. Each component was dissolved one at a time in Di H₂O, pH to 3.0 with HCl/NaOH, then q.s. to volume, and filter sterilized with a 0.22 micron filter.

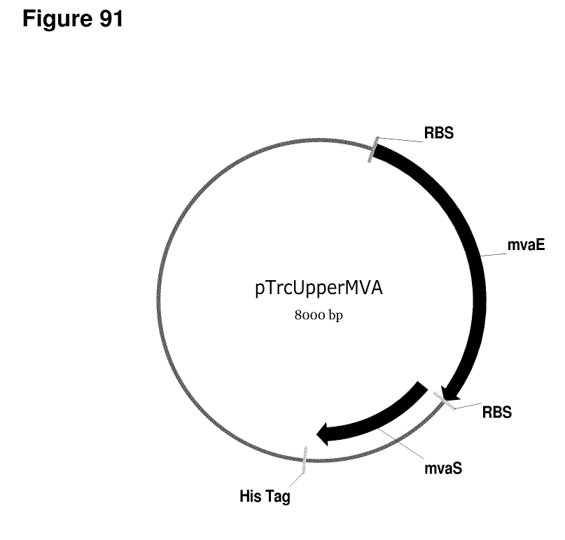

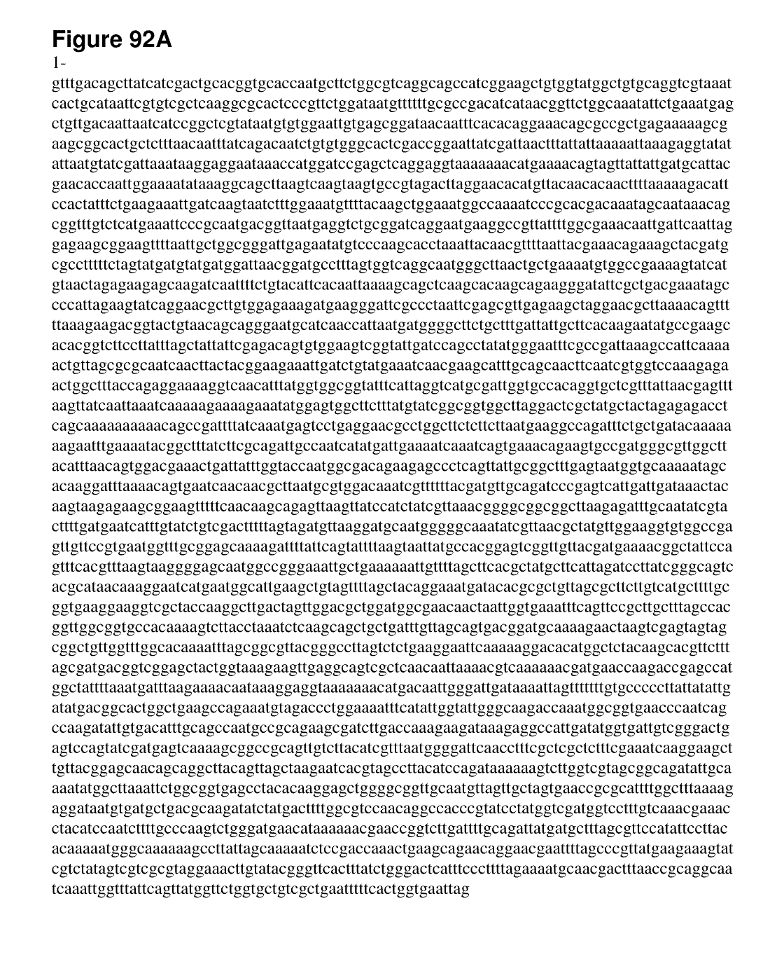

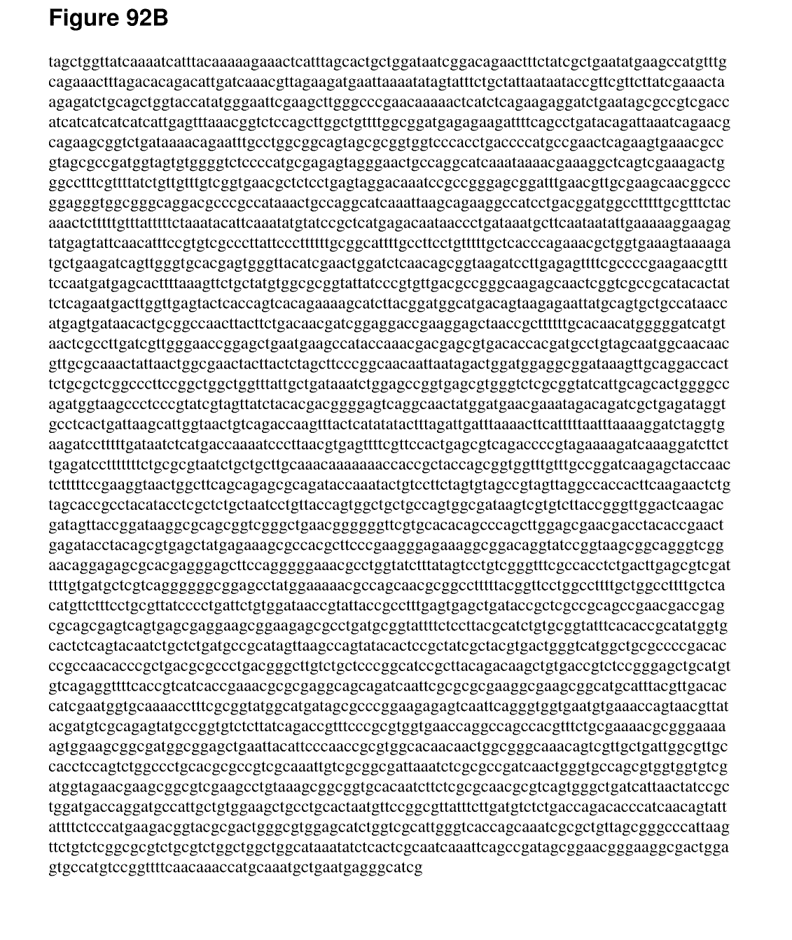

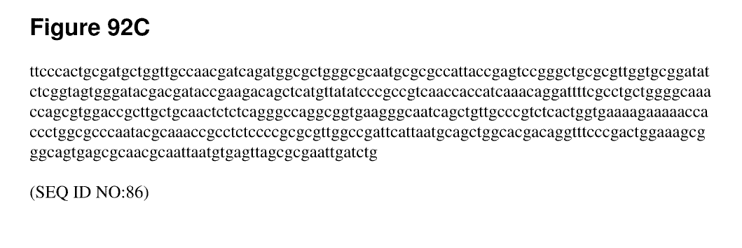

Fermentation was performed with E. coli cells containing the pTrcHis2AUpperPathway (also called pTrcUpperMVA, FIGS. 91 and 92A-92C) (50 μg/ml carbenicillin) or the pCL PtrcUpperMVA (also called pCL PtrcUpperPathway (FIG. 26)) (50 μg/ml spectinomycin) plasmids. For experiments in which isoprene was produced, the E. coli cells also contained the pTrc KKDyIkIS (50 μg/ml kanamycin) plasmid. These experiments were carried out to monitor mevalonic acid or isoprene formation from glucose at the desired fermentation pH 7.0 and temperature 30° C. An inoculum of an E. coli strain taken from a frozen vial was streaked onto an LA broth agar plate (with antibiotics) and incubated at 37° C. A single colony was inoculated into tryptone-yeast extract medium. After the inoculum grew to optical density 1.0 when measured at 550 nm, it was used to inoculate the bioreactor.

Glucose was fed at an exponential rate until cells reached the stationary phase. After this time the glucose feed was decreased to meet metabolic demands. Induction was achieved by adding IPTG. The mevalonic acid concentration in fermentation broth was determined by applying perchloric acid (Sigma-Aldrich #244252) treated samples (0.3 M incubated at 4° C. for 5 minutes) to an organic acids HPLC column (BioRad #125-0140). The concentration was determined by comparing the broth mevalonic acid peak size to a calibration curve generated from mevalonolacetone (Sigma-Aldrich #M4667) treated with perchloric acid to form D,L-mevalonate. The isoprene level in the off gas from the bioreactor was determined as described herein. The isoprene titer is defined as the amount of isoprene produced per liter of fermentation broth.

II. Mevalonic Acid Production from E. coli BL21 (DE3) Cells Expressing the pTrcUpperMVA Plasmid at a 150-L Scale

BL21 (DE3) cells that were grown on a plate as explained above in Example 22, part I were inoculated into a flask containing 45 mL of tryptone-yeast extract medium and incubated at 30° C. with shaking at 170 rpm for 5 hours. This solution was transferred to a 5-L bioreactor of tryptone-yeast extract medium, and the cells were grown at 30° C. and 27.5 rpm until the culture reached an OD₅₅₀ of 1.0. The 5 L of inoculum was seeded into a 150-L bioreactor containing 45-kg of medium. The IPTG concentration was brought to 1.1 mM when the OD₅₅₀ reached a value of 10. The OD₅₅₀ profile within the bioreactor over time is shown in FIG. 60A. The mevalonic acid titer increased over the course of the fermentation to a final value of 61.3 g/L (FIG. 60B). The specific productivity profile throughout the fermentation is shown in FIG. 60C and a comparison to FIG. 60A illustrates the de-coupling of growth and mevalonic acid production. The total amount of mevalonic acid produced during the 52.5 hour fermentation was 4.0 kg from 14.1 kg of utilized glucose. The molar yield of utilized carbon that went into producing mevalonic acid during fermentation was 34.2%.

III. Mevalonic Acid Production from E. coli BL21 (DE3) Cells Expressing the pTrcUpperMVA Plasmid at a 15-L Scale

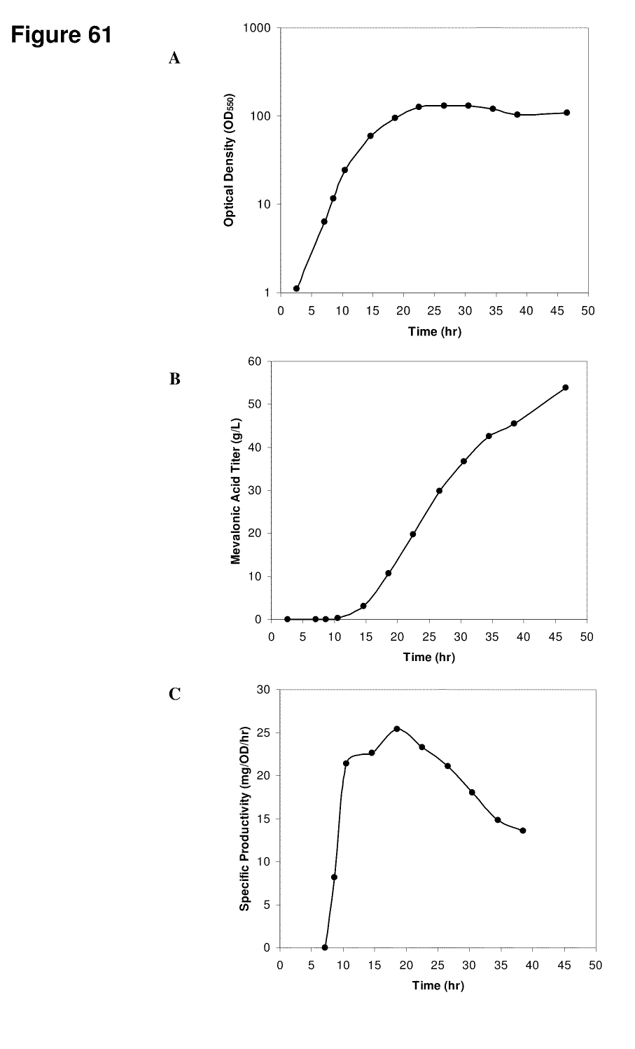

BL21 (DE3) cells that were grown on a plate as explained above in Example 22, part I were inoculated into a flask containing 500 mL of tryptone-yeast extract medium and grown at 30° C. at 160 rpm to OD₅₅₀ 1.0. This material was seeded into a 15-L bioreactor containing 4.5-kg of medium. The IPTG concentration was brought to 1.0 mM when the OD₅₅₀ reached a value of 10. The OD₅₅₀ profile within the bioreactor over time is shown in FIG. 61A. The mevalonic acid titer increased over the course of the fermentation to a final value of 53.9 g/L (FIG. 61B). The specific productivity profile throughout the fermentation is shown in FIG. 61C and a comparison to FIG. 61A illustrates the de-coupling of growth and mevalonic acid production. The total amount of mevalonic acid produced during the 46.6 hour fermentation was 491 g from 2.1 kg of utilized glucose. The molar yield of utilized carbon that went into producing mevalonic acid during fermentation was 28.8%.

IV. Mevalonic Acid Production from E. coli FM5 Cells Expressing the pTrcUpperMVA Plasmid at a 15-L Scale

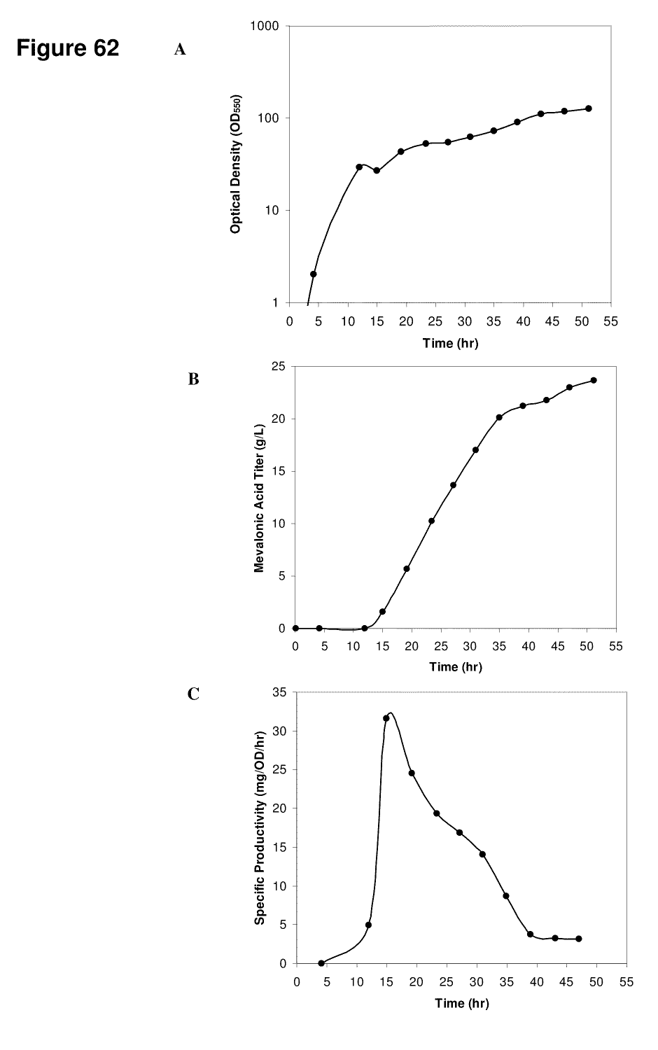

FM5 cells that were grown on a plate as explained above in Example 22, part I were inoculated into a flask containing 500 mL of tryptone-yeast extract medium and grown at 30° C. at 160 rpm to OD₅₅₀ 1.0. This material was seeded into a 15-L bioreactor containing 4.5-kg of medium. The IPTG concentration was brought to 1.0 mM when the OD₅₅₀ reached a value of 30. The OD₅₅₀ profile within the bioreactor over time is shown in FIG. 62A. The mevalonic acid titer increased over the course of the fermentation to a final value of 23.7 g/L (FIG. 62B). The specific productivity profile throughout the fermentation is shown in FIG. 62C and a comparison to FIG. 62A illustrates the de-coupling of growth and mevalonic acid production. The total amount of mevalonic acid produced during the 51.2 hour fermentation was 140 g from 1.1 kg of utilized glucose. The molar yield of utilized carbon that went into producing mevalonic acid during fermentation was 15.2%.

V. Isoprene Production from E. coli BL21 (DE3) Cells Expressing the pCL PtrcUpperMVA and pTrc KKDyIkIS Plasmids at a 15-L Scale

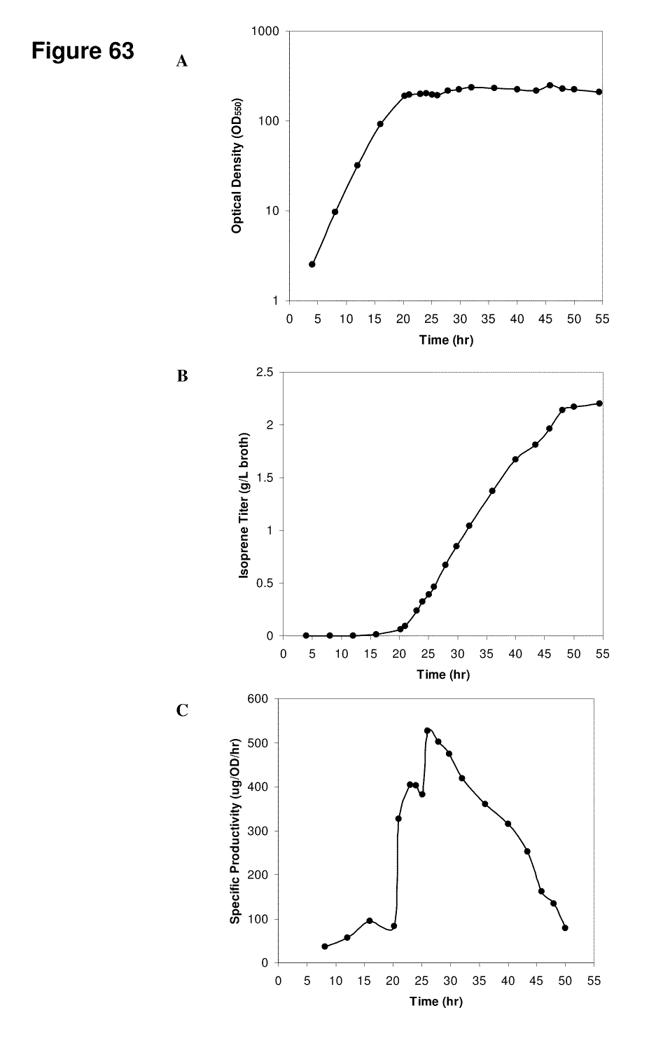

BL21 (DE3) cells expressing the pCL PtrcUpperMVA and pTrc KKDyIkIS plasmids that were grown on a plate as explained above in Example 22, part I were inoculated into a flask containing 500 mL of tryptone-yeast extract medium and grown at 30° C. at 160 rpm to OD₅₅₀ 1.0. This material was seeded into a 15-L bioreactor containing 4.5-kg of medium. The IPTG concentration was brought to 25 μM when the OD₅₅₀ reached a value of 10. The IPTG concentration was raised to 50 uM when OD₅₅₀ reached 190. The IPTG concentration was raised to 100 uM at 38 hours of fermentation. The OD₅₅₀ profile within the bioreactor over time is shown in FIG. 63A. The isoprene titer increased over the course of the fermentation to a final value of 2.2 g/L broth (FIG. 63B). The specific productivity profile throughout the fermentation is shown in FIG. 63C and a comparison to FIG. 63A illustrates the de-coupling of growth and isoprene production. The total amount of isoprene produced during the 54.4 hour fermentation was 15.9 g from 2.3 kg of utilized glucose. The molar yield of utilized carbon that went into producing isoprene during fermentation was 1.53%.

VI. Isoprene Production from E. coli BL21 (DE3) Tuner Cells Expressing the pCL PtrcUpperMVA and pTrc KKDyIkIS Plasmids at a 15-L Scale

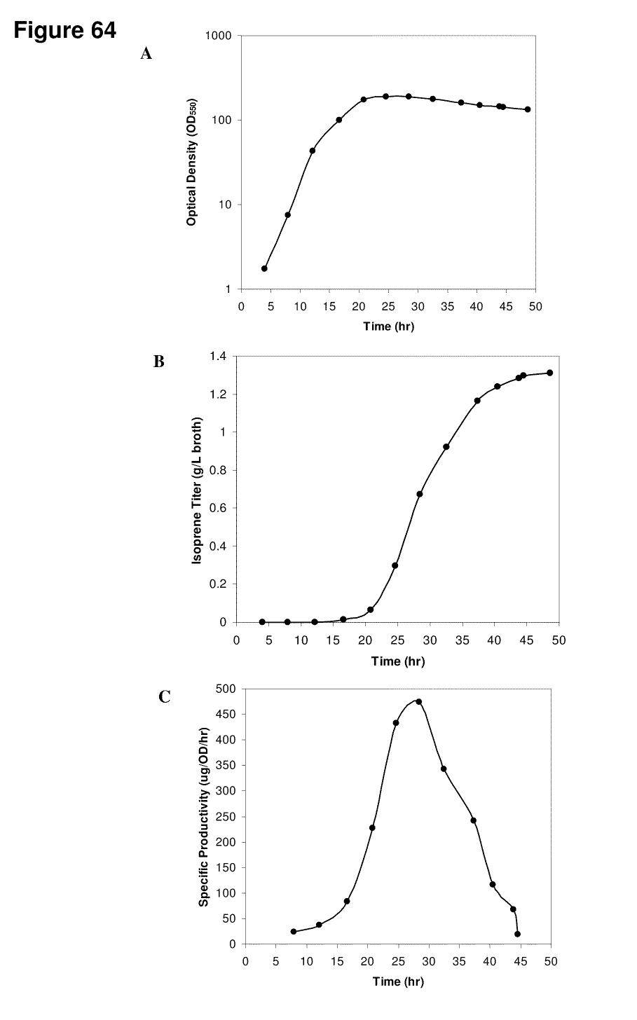

BL21 (DE3) tuner cells expressing the pCL PtrcUpperMVA and pTrc KKDyIkIS plasmids that were grown on a plate as explained above in Example 22, part I were inoculated into a flask containing 500 mL of tryptone-yeast extract medium and grown at 30° C. at 160 rpm to OD₅₅₀ 1.0. This material was seeded into a 15-L bioreactor containing 4.5-kg of medium. The IPTG concentration was brought to 26 μM when the OD₅₅₀ reached a value of 10. The IPTG concentration was raised to 50 uM when OD₅₅₀ reached 175. The OD₅₅₀ profile within the bioreactor over time is shown in FIG. 64A. The isoprene titer increased over the course of the fermentation to a final value of 1.3 g/L broth (FIG. 64B). The specific productivity profile throughout the fermentation is shown in FIG. 64C and a comparison to FIG. 64A illustrates the de-coupling of growth and isoprene production. The total amount of isoprene produced during the 48.6 hour fermentation was 9.9 g from 1.6 kg of utilized glucose. The molar yield of utilized carbon that went into producing isoprene during fermentation was 1.34%.

VII. Isoprene Production from E. coli MG1655 Cells Expressing the pCL PtrcUpperMVA and pTrc KKDyIkIS Plasmids at a 15-L Scale

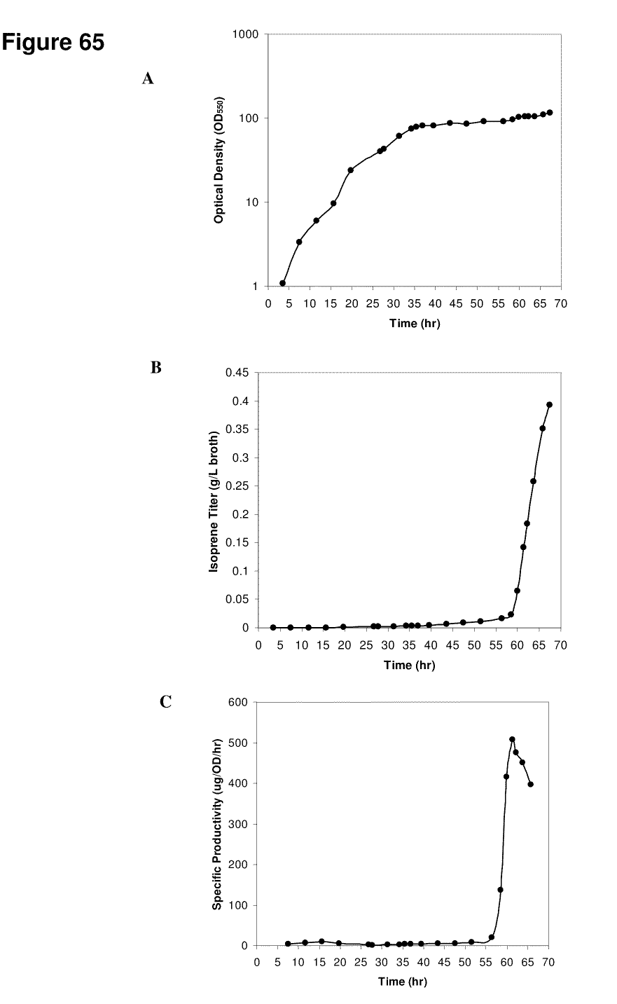

MG1655 cells expressing the pCL PtrcUpperMVA and pTrc KKDyIkIS plasmids that were grown on a plate as explained above in Example 22, part I were inoculated into a flask containing 500 mL of tryptone-yeast extract medium and grown at 30° C. at 160 rpm to OD₅₅₀ 1.0. This material was seeded into a 15-L bioreactor containing 4.5-kg of medium. The IPTG concentration was brought to 24 μM when the OD₅₅₀ reached a value of 45. The OD₅₅₀ profile within the bioreactor over time is shown in FIG. 65A. The isoprene titer increased over the course of the fermentation to a final value of 393 mg/L broth (FIG. 65B). The specific productivity profile throughout the fermentation is shown in FIG. 65C and a comparison to FIG. 65A illustrates the de-coupling of growth and isoprene production. The total amount of isoprene produced during the 67.4 hour fermentation was 2.2 g from 520 g of utilized glucose. The molar yield of utilized carbon that went into producing isoprene during fermentation was 0.92%.

VIII. Isoprene Production from E. coli MG1655ack-pta Cells Expressing the pCL PtrcUpperMVA and pTrc KKDyIkIS Plasmids at a 15-L Scale

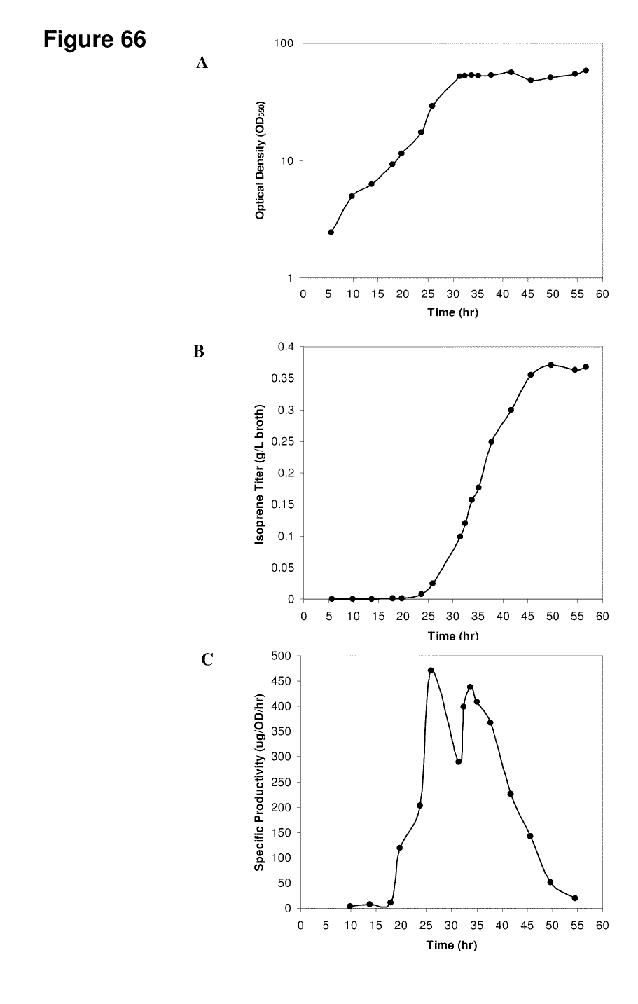

MG1655ack-pta cells expressing the pCL PtrcUpperMVA and pTrc KKDyIkIS plasmids that were grown on a plate as explained above in Example 22, part I were inoculated into a flask containing 500 mL of tryptone-yeast extract medium and grown at 30° C. at 160 rpm to OD₅₅₀ 1.0. This material was seeded into a 15-L bioreactor containing 4.5-kg of medium. The IPTG concentration was brought to 30 μM when the OD₅₅₀ reached a value of 10. The OD₅₅₀ profile within the bioreactor over time is shown in FIG. 66A. The isoprene titer increased over the course of the fermentation to a final value of 368 mg/L broth (FIG. 66B). The specific productivity profile throughout the fermentation is shown in FIG. 66C and a comparison to FIG. 66A illustrates the de-coupling of growth and isoprene production. The total amount of isoprene produced during the 56.7 hour fermentation was 1.8 g from 531 g of utilized glucose. The molar yield of utilized carbon that went into producing isoprene during fermentation was 0.73%.

IX. Isoprene Production from E. coli FM5 Cells Expressing the pCL PtrcUpperMVA and pTrc KKDyIkIS Plasmids at a 15-L Scale

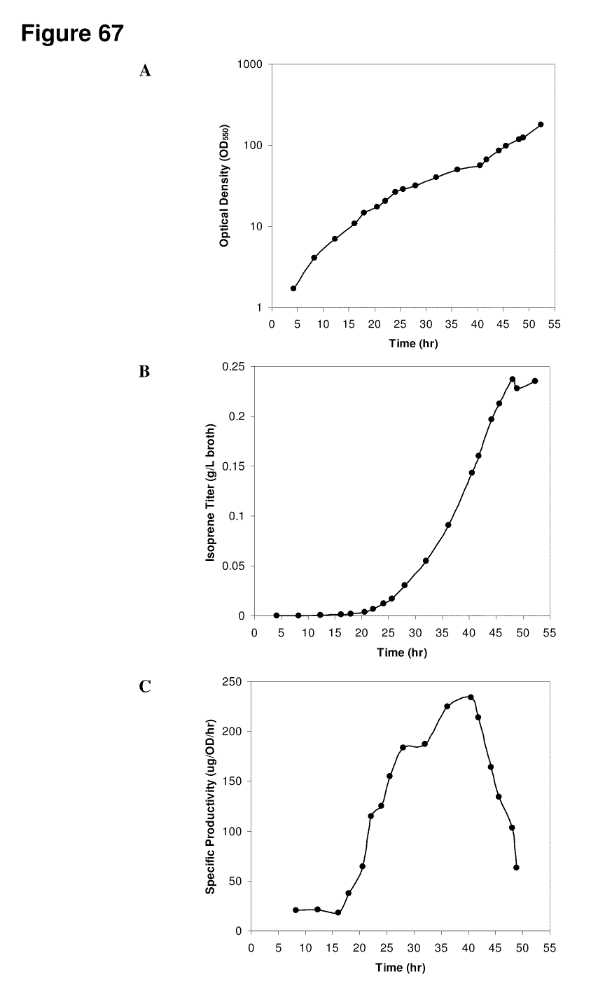

FM5 cells expressing the pCL PtrcUpperMVA and pTrc KKDyIkIS plasmids that were grown on a plate as explained above in Example 22, part I were inoculated into a flask containing 500 mL of tryptone-yeast extract medium and grown at 30° C. at 160 rpm to OD₅₅₀ 1.0. This material was seeded into a 15-L bioreactor containing 4.5-kg of medium. The IPTG concentration was brought to 27 μM when the OD₅₅₀ reached a value of 15. The OD₅₅₀ profile within the bioreactor over time is shown in FIG. 67A. The isoprene titer increased over the course of the fermentation to a final value of 235 mg/L broth (FIG. 67B). The specific productivity profile throughout the fermentation is shown in FIG. 67C and a comparison to FIG. 67A illustrates the de-coupling of growth and isoprene production. The total amount of isoprene produced during the 52.3 hour fermentation was 1.4 g from 948 g of utilized glucose. The molar yield of utilized carbon that went into producing isoprene during fermentation was 0.32%.

### Example 23

### Production of Isoprene During the Exponential Growth Phase of E. coli Expressing Genes from the Mevalonic Acid Pathway and Fermented in a Fed-Batch Culture

This example illustrates the production of isoprene during the exponential growth phase of cells.

Medium Recipe (Per Liter Fermentation Medium):

The medium was generated using the following components per liter fermentation medium: K₂HPO₄ 7.5 g, MgSO₄*7H₂O 2 g, citric acid monohydrate 2 g, ferric ammonium citrate 0.3 g, yeast extract 0.5 g, and 1000× modified trace metal solution 1 ml. All of the components were added together and dissolved in diH₂O. This solution was autoclaved. The pH was adjusted to 7.0 with ammonium hydroxide (30%) and q.s. to volume. Glucose 10 g, thiamine*HCl 0.1 g, and antibiotics were added after sterilization and pH adjustment.

1000× Modified Trace Metal Solution:

The 1000× modified trace metal solution was generated using the following components: citric acids*H₂O 40 g, MnSO₄*H₂O 30 g, NaCl 10 g, FeSO₄*7H₂O 1 g, CoCl2*6H₂O 1 g, ZnSO₄*7H₂O 1 g, CuSO₄*5H₂O 100 mg, H₃BO₃ 100 mg, and NaMoO₄*2H₂O 100 mg. Each component is dissolved one at a time in Di H2O, pH to 3.0 with HC1/NaOH, then q.s. to volume and filter sterilized with 0.22 micron filter.

Fermentation was performed in a 15-L bioreactor with ATCC11303 E. coli cells containing the pCL PtrcUpperMVA and pTrc KKDyIkIS plasmids. This experiment was carried out to monitor isoprene formation from glucose at the desired fermentation pH 7.0 and temperature 30° C. An inoculum of E. coli strain taken from a frozen vial was streaked onto an LB broth agar plate (with antibiotics) and incubated at 37° C. A single colony was inoculated into tryptone-yeast extract medium. After the inoculum grew to OD 1.0, measured at 550 nm, 500 mL was used to inoculate a 15-L bioreactor containing an initial working volume of 5 L.

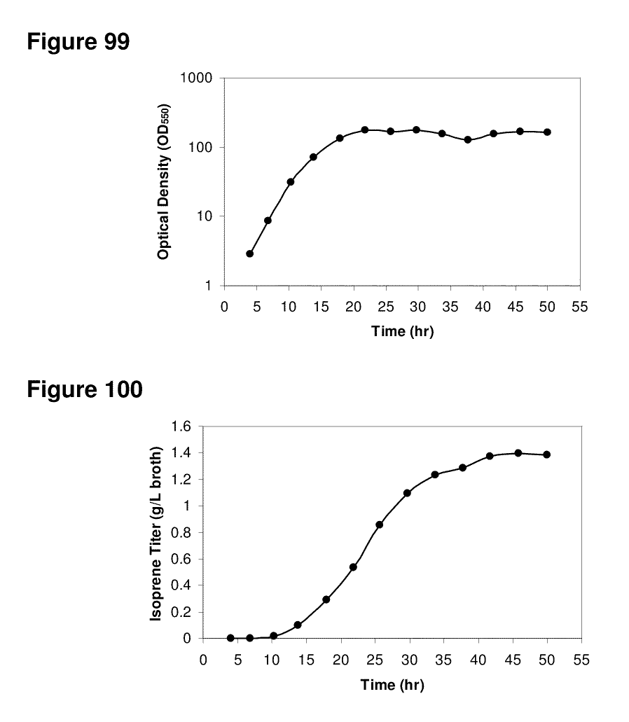

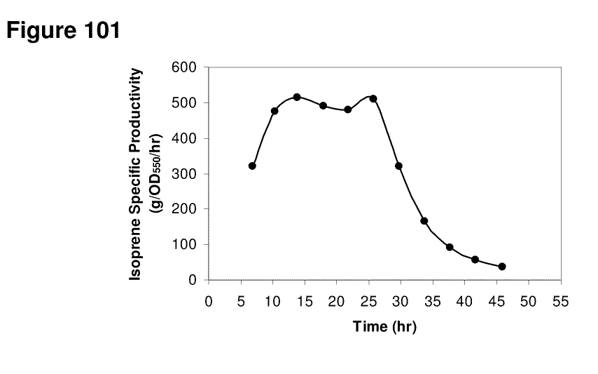

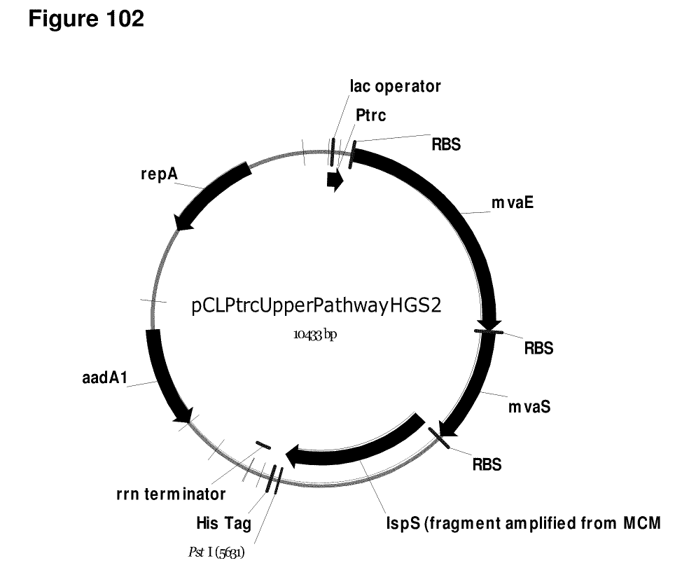

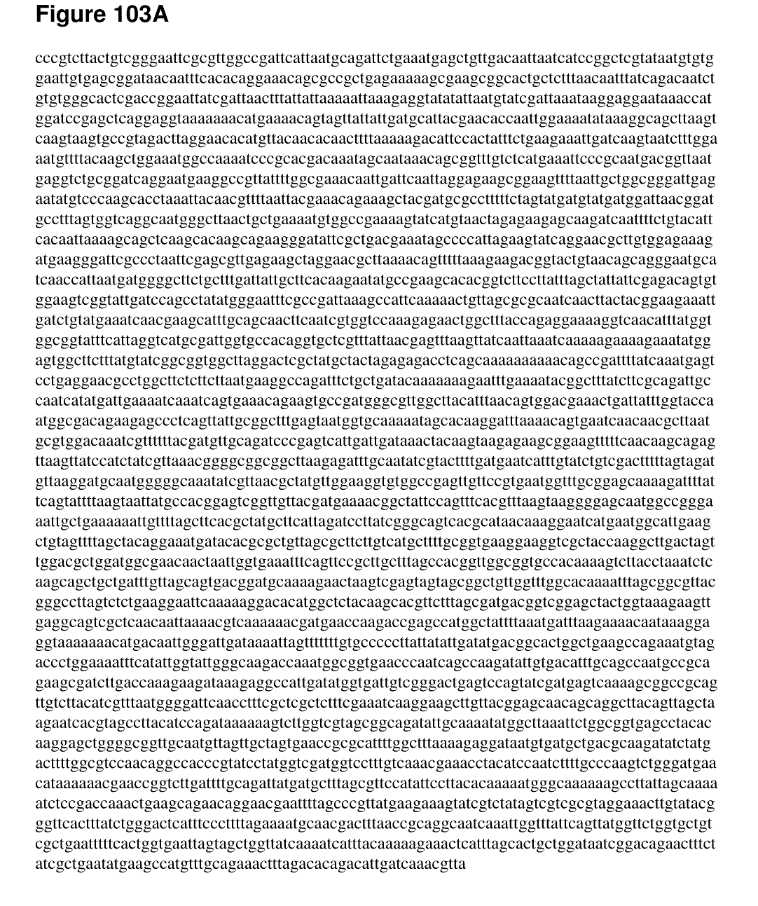

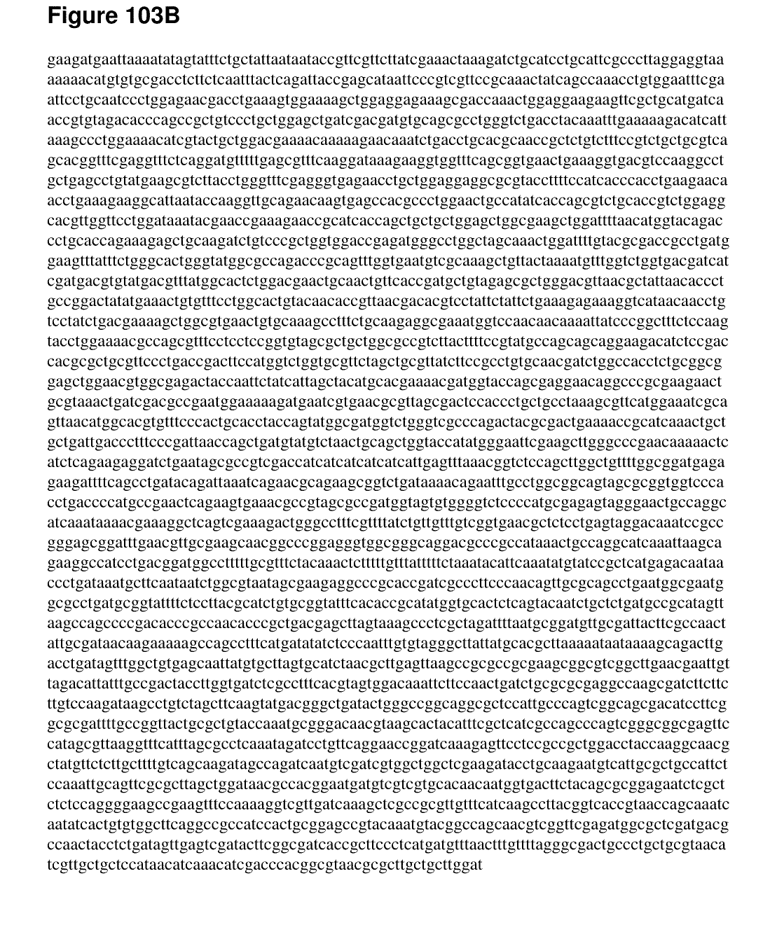

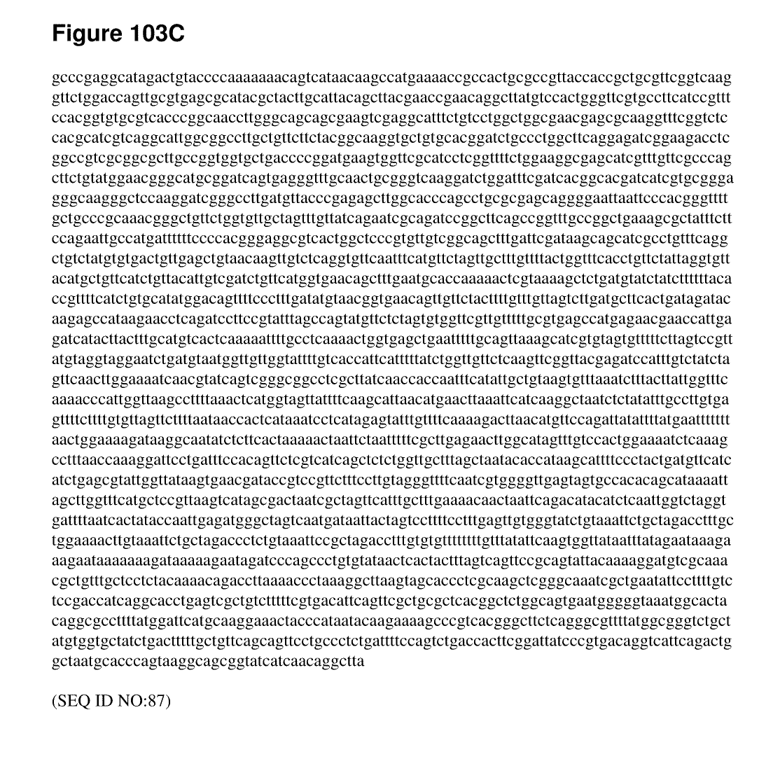

Glucose was fed at an exponential rate until cells reached the stationary phase. After this time the glucose feed was decreased to meet metabolic demands. The total amount of glucose delivered to the bioreactor during the 50 hour fermentation was 2.0 kg. Induction was achieved by adding IPTG. The IPTG concentration was brought to 25 uM when the optical density at 550 nm (OD₅₅₀) reached a value of 10. The IPTG concentration was raised to 50 uM when OD₅₅₀ reached 190. The OD₅₅₀ profile within the bioreactor over time is shown in FIG. 99. The isoprene level in the off gas from the bioreactor was determined as described herein. The isoprene titer increased over the course of the fermentation to a final value of 1.4 g/L (FIG. 100). The total amount of isoprene produced during the 50 hour fermentation was 10.0 g. The profile of the isoprene specific productivity over time within the bioreactor is shown in FIG. 101. The molar yield of utilized carbon that contributed to producing isoprene during fermentation was 1.1%. The weight percent yield of isoprene from glucose was 0.5%.

### Example 24

### Flammability Modeling and Testing of Isoprene

### I. Summary of Flammability Modeling and Testing of Isoprene

Flammability modeling and experiments were performed for various hydrocarbon/oxygen/nitrogen/water/carbon dioxide mixtures. This modeling and experimental tested was aimed at defining isoprene and oxygen/nitrogen flammability curves under specified steam and carbon monoxide concentrations at a fixed pressure and temperature. A matrix of the model conditions is shown in Table 13, and a matrix of the experiments performed is shown in Table 14.

TABLE 13
Summary of Modeled Isoprene Flammability

| | | | Steam | Carbon Dioxide | Isoprene | Oxygen |
|---|---|---|---|---|---|---|
| | Temperature | Pressure | Concentration | Concentration | Concentration | Concentration |
| Series | (° C.) | (psig) | (wt %) | (wt. %) | (vol. %) | (vol. %) |
| A | 40 | 0 | 0 | 0 | Varying | Varying |
| B | 40 | 0 | 4 | 0 | Varying | Varying |
| C | 40 | 0 | 0 | 5 | Varying | Varying |
| D | 40 | 0 | 0 | 10 | Varying | Varying |
| E | 40 | 0 | 0 | 15 | Varying | Varying |
| F | 40 | 0 | 0 | 20 | Varying | Varying |
| G | 40 | 0 | 0 | 30 | Varying | Varying |

TABLE 14
Summary of Isoprene Flammability Tests

| | | | Steam | Isoprene | Oxygen |
|---|---|---|---|---|---|
| Series | Temperature | Pressure | Concentration | Concentration | Concentration |
| Number | (° C.) | (psig) | (vol. %) | (vol. %) | (vol. %) |
| 1 | 40 | 0 | 0 | Varying | Varying |
| 2 | 40 | 0 | 4 | Varying | Varying |

### II. Description of Calculated Adiabatic Flame Temperature (CAFT) Model

Calculated adiabatic flame temperatures (CAFT) along with a selected limit flame temperature for combustion propagation were used to determine the flammability envelope for isoprene. The computer program used in this study to calculate the flame temperatures is the NASA Glenn Research Center CEA (Chemical Equilibrium with Applications) software.

There are five steps involved in determining the flammability envelope using an adiabatic flame temperature model for a homogeneous combustion mechanism (where both the fuel and oxidant are in the gaseous state): selection of the desired reactants, selection of the test condition, selection of the limit flame temperature, modification of the reactants, and construction of a flammability envelope from calculations.

In this first step, selection of desired reactants, a decision must be made as to the reactant species that will be present in the system and the quantities of each. In many cases the computer programs used for the calculations have a list of reactant and product species. If any of the data for the species to be studied are not found in the program, they may be obtained from other sources such as the JANAF tables or from the internet. In this current model data for water, nitrogen, oxygen and carbon dioxide were present in the program database. The program database did not have isoprene as a species; therefore the thermodynamic properties were incorporated manually.

The next step is to decide whether the initial pressure and temperature conditions that the combustion process is taking place in. In this model the pressure was 1 atmosphere (absolute) and the temperature was 40° C., the boiling point of isoprene.

The limit flame temperature for combustion can be either selected based on theoretical principles or determined experimentally. Each method has its own limitations.

Based on prior studies, the limit flame temperatures of hydrocarbons fall in the range of 1000 K to 1500 K. For this model, the value of 1500 K was selected. This is the temperature at which the reaction of carbon monoxide to carbon dioxide (a highly exothermic reaction and constitutes a significant proportion of the flame energy) becomes self sustaining.

Once the limit flame temperature has been decided upon, model calculations are performed on the given reactant mixture (species concentrations) and the adiabatic flame temperature is determined. Flame propagation is considered to have occurred only if the temperature is greater than the limit flame temperature. The reactant mixture composition is then modified to create data sets for propagation and non-propagation mixtures.

This type of model shows good agreement with the experimentally determined flammability limits. Regions outside the derived envelope are nonflammable and regions within it are flammable. The shape of the envelope forms a nose. The nose of the envelope is related to the limiting oxygen concentration (LOC) for gaseous fuels.

III. Results from Calculated Adiabatic Flame Temperature (CAFT) Model

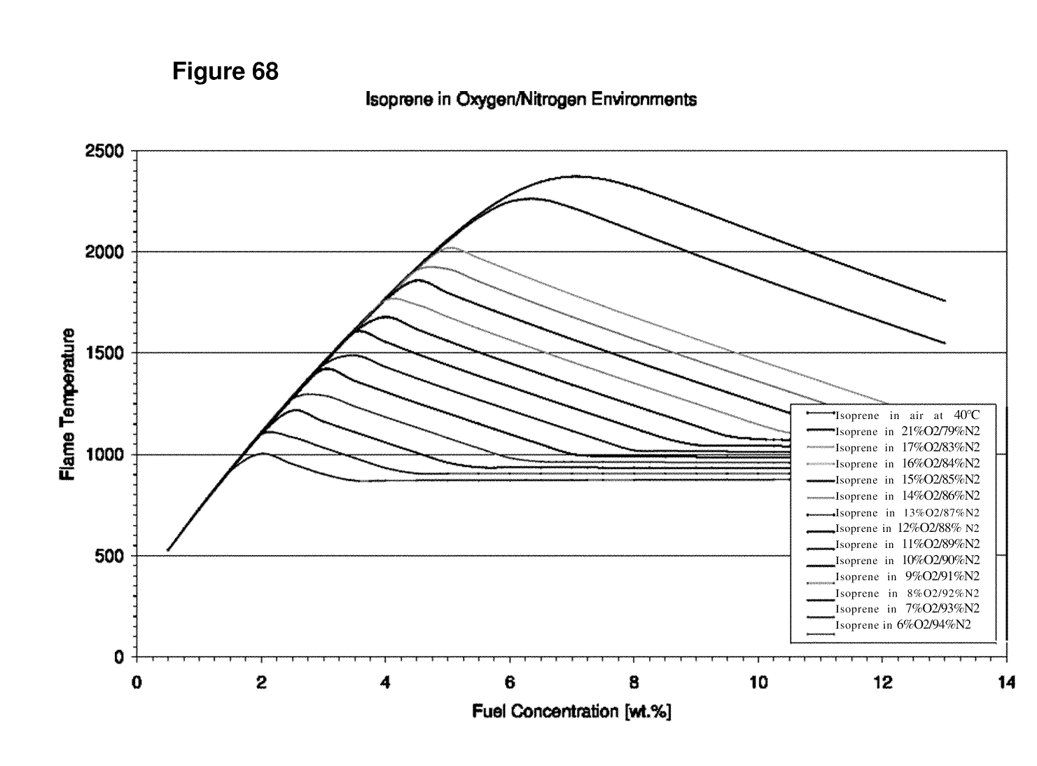

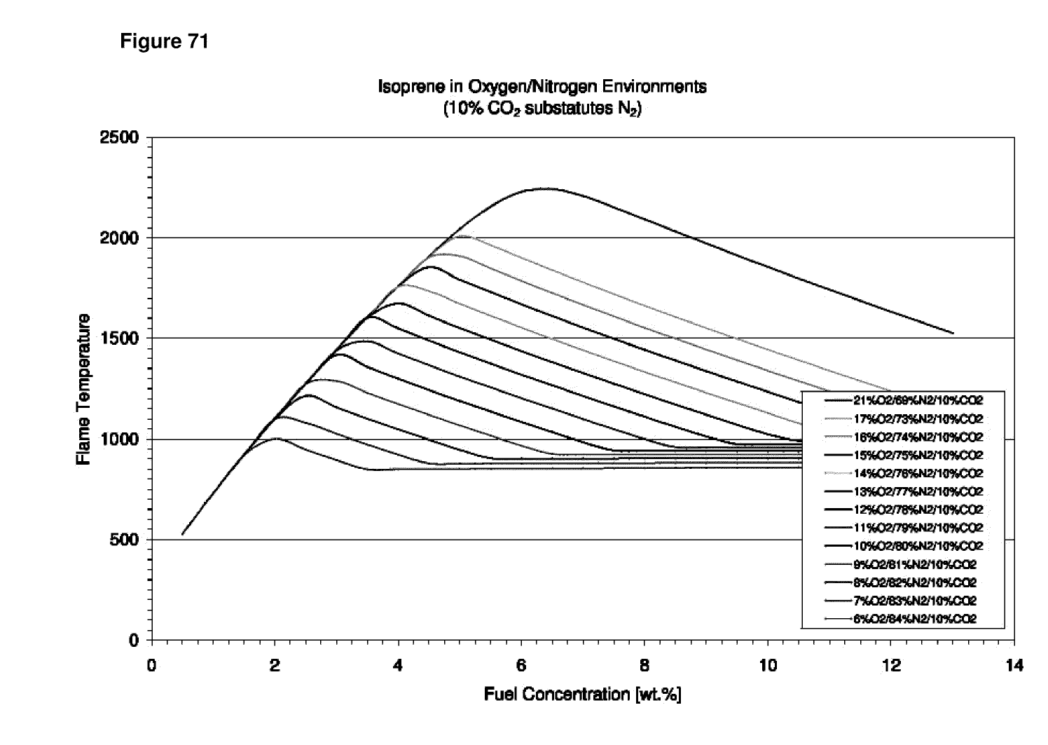

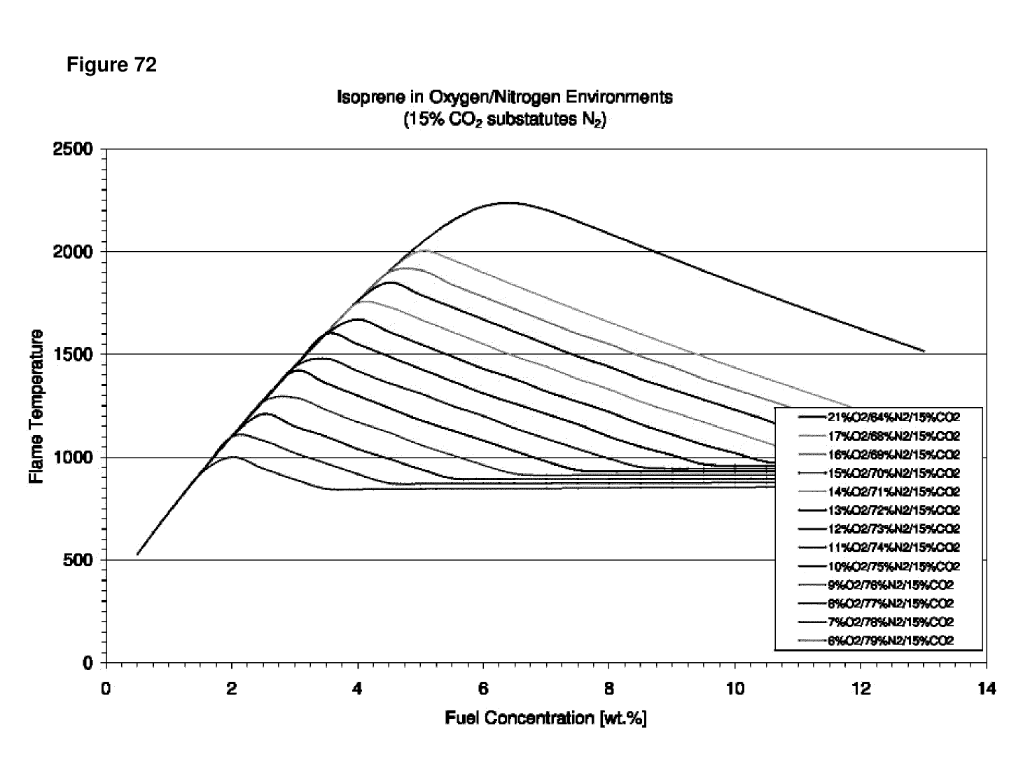

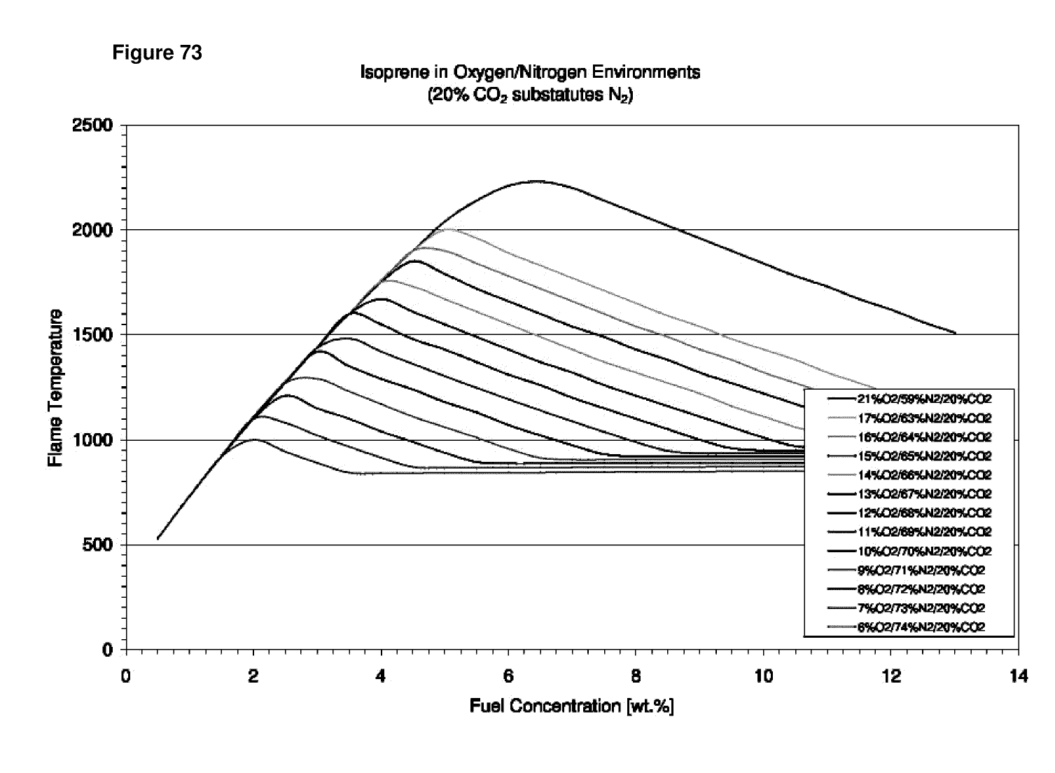

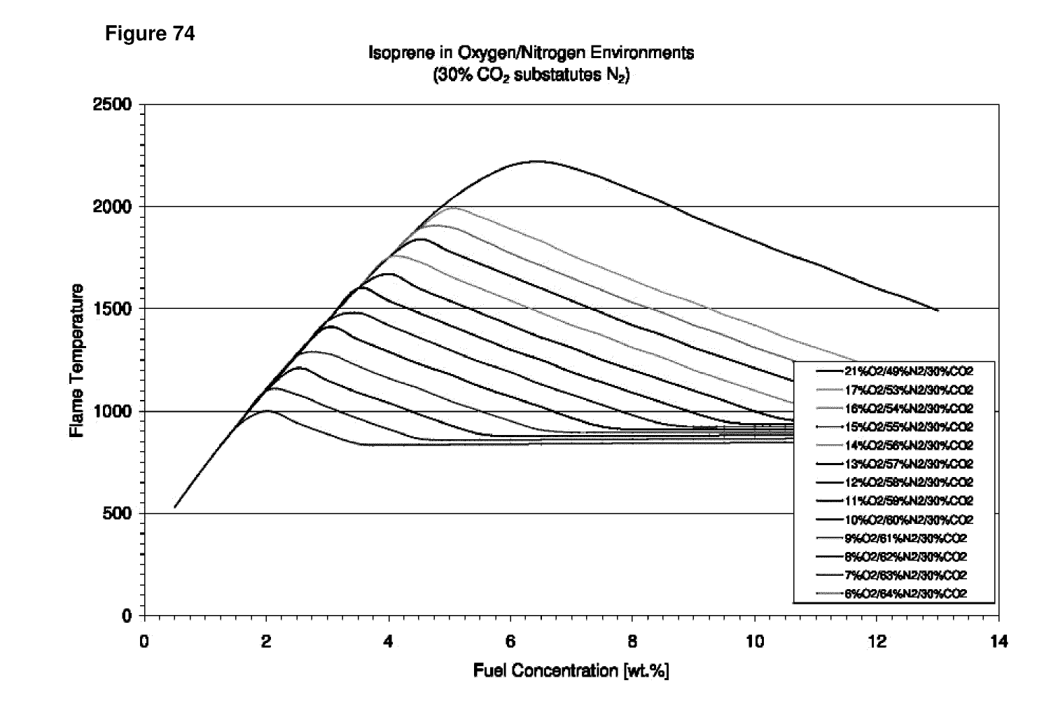

Plotted in FIGS. 68 through 74 are the CAFT model results for Series A to G, respectively. The figures plot the calculated adiabatic flame temperature (using the NASA CEA program) as a function of fuel concentration (by weight) for several oxygen/nitrogen ratios (by weight). The parts of the curve that are above 1500 K, the selected limit flame temperature, contain fuel levels sufficient for flame propagation. The results may be difficult to interpret in the form presented in FIGS. 68 through 74. Additionally, the current form is not conducive to comparison with experimental data which is generally presented in terms of volume percent.

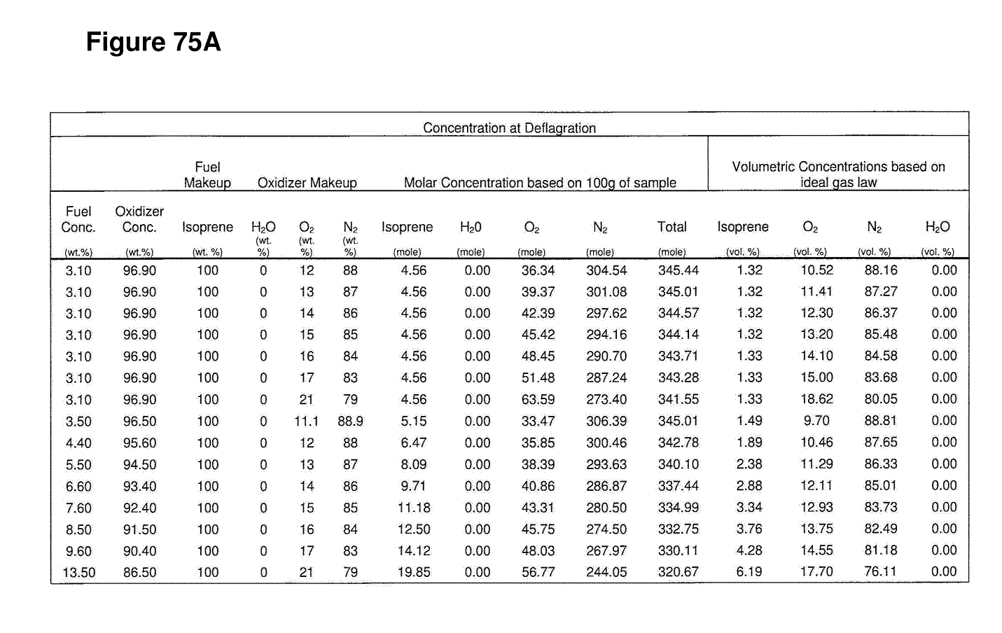

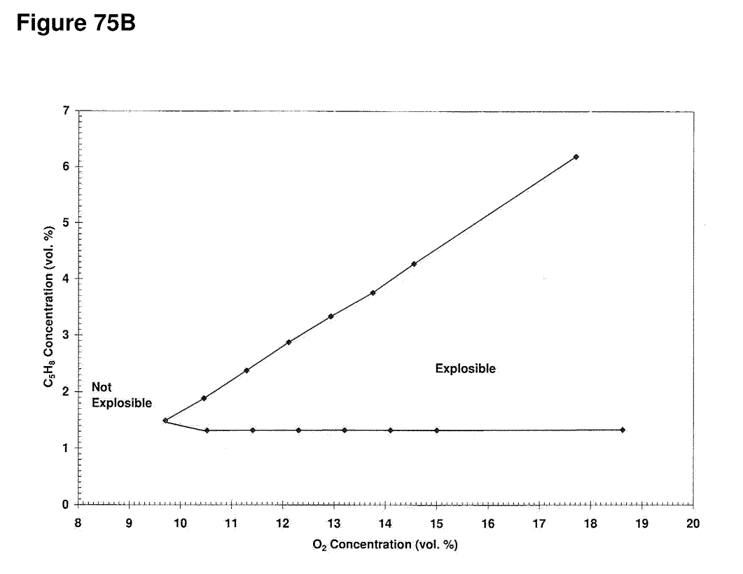

Using Series A as an example the data in FIG. 68 can be plotted in the form of a traditional flammability envelope. Using FIG. 68 and reading across the 1500 K temperature line on the ordinate one can determine the fuel concentration for this limit flame temperature by dropping a tangent to the abscissa for each curve (oxygen to nitrogen ratio) that it intersects. These values can then be tabulated as weight percent of fuel for a given weight percent of oxidizer (FIG. 75A). Then knowing the composition of the fuel (100 wt. % isoprene) and the composition of the oxidizer (relative content of water, oxygen and nitrogen) molar quantities can be established.

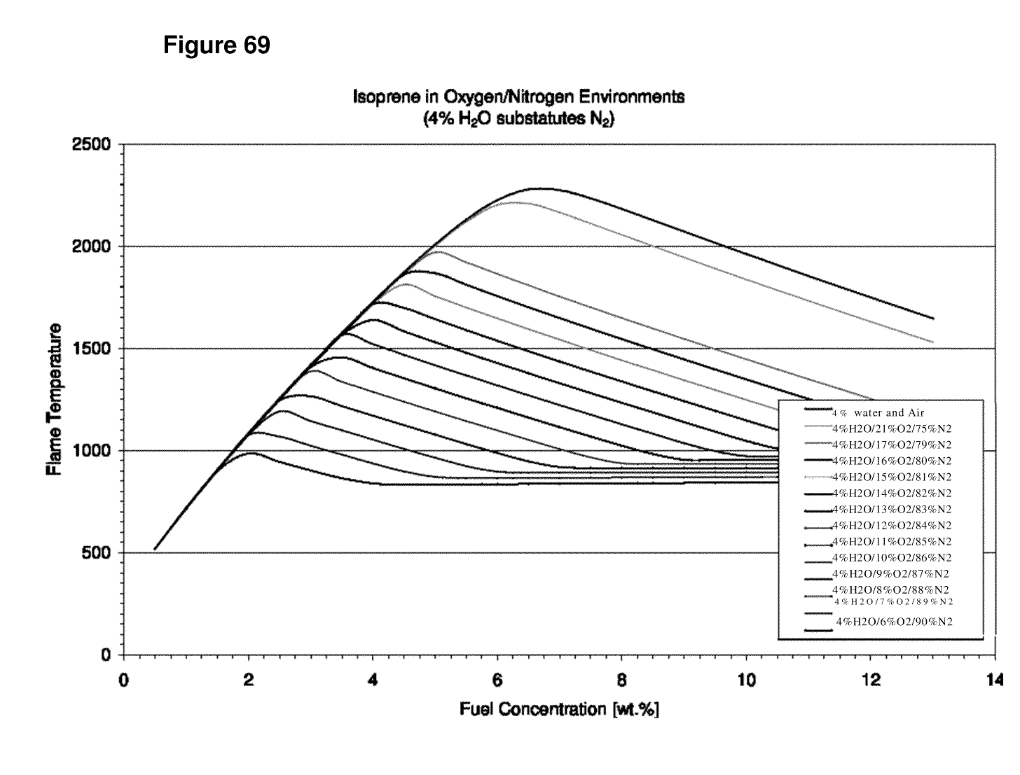

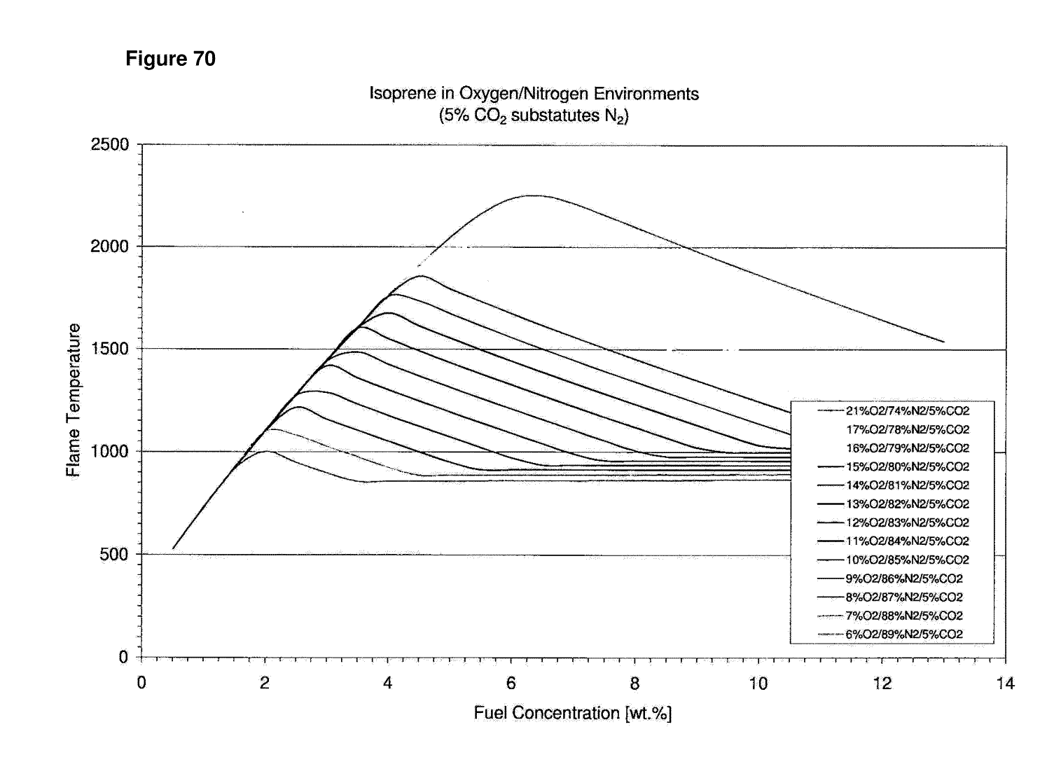

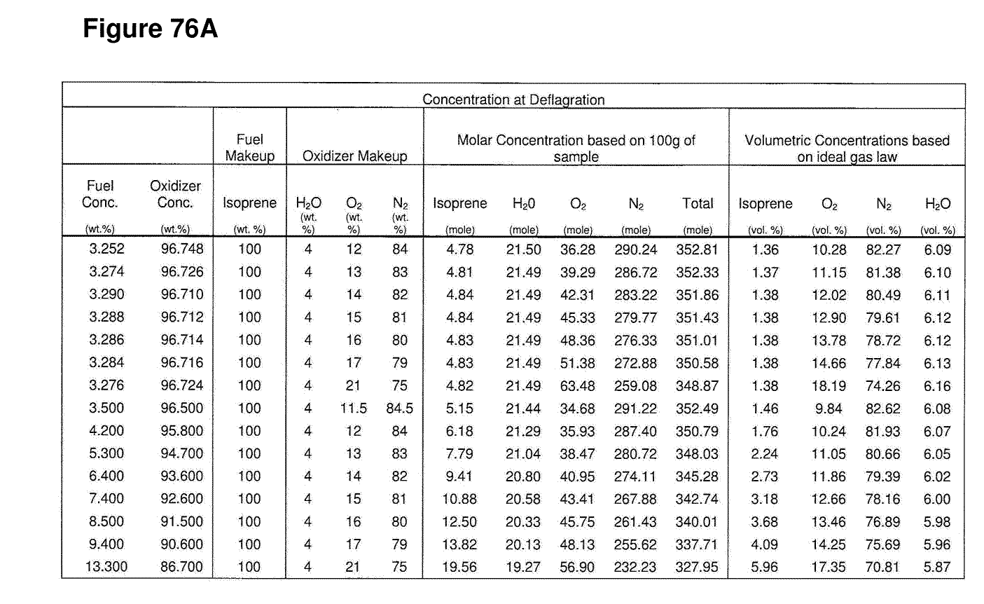

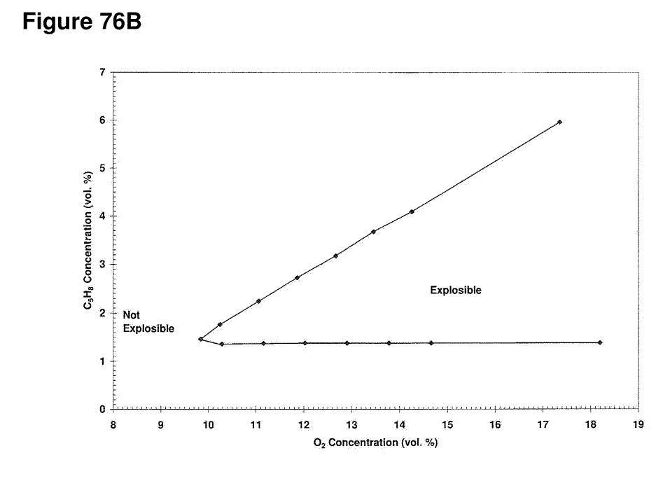

From these molar quantities percentage volume concentrations can be calculated. The concentrations in terms of volume percent can then be plotted to generate a flammability envelope (FIG. 75B). The area bounded by the envelope is the explosible range and the area excluded is the non-explosible range. The “nose” of the envelope is the limiting oxygen concentration. FIGS. 76A and 76B contain the calculated volume concentrations for the flammability envelope for Series B generated from data presented in FIG. 69. A similar approach can be used on data presented in FIGS. 70-74.

### IV. Flammability Testing Experimental Equipment and Procedure

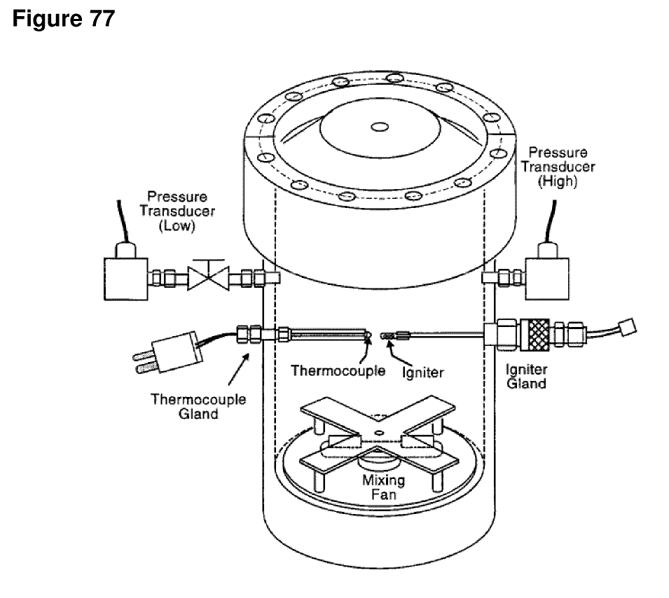

Flammability testing was conducted in a 4 liter high pressure vessel. The vessel was cylindrical in shape with an inner diameter of 6″ and an internal height of 8.625″. The temperature of the vessel (and the gases inside) was maintained using external heaters that were controlled by a PID controller. To prevent heat losses, ceramic wool and reflective insulation were wrapped around the pressure vessel. Type K thermocouples were used the measure the temperature of the gas space as well as the temperature of the vessel itself. FIG. 77 illustrates the test vessel.

Before a test was ran, the vessel was evacuated and purged with nitrogen to ensure that any gases from previous tests were removed. A vacuum was then pulled on the vessel. The pressure after this had been done was typically around 0.06 bar(a). Due to the nitrogen purging, the gas responsible for this initial pressure was assumed to be nitrogen. Using partial pressures, water, isoprene, nitrogen, and oxygen were then added in the appropriate amounts to achieve the test conditions in question. A magnetically driven mixing fan within the vessel ensured mixing of the gaseous contents. The gases were allowed to mix for about 2 minutes with the fan being turned off approximately 1 minute prior to ignition.

The igniter was comprised of a 1.5 ohm nicrome coil and an AC voltage source on a timer circuit. Using an oscilloscope, it was determined that 34.4 VAC were delivered to the igniter for 3.2 seconds. A maximum current of 3.8 amps occurred approximately halfway into the ignition cycle. Thus, the maximum power was 131 W and the total energy provided over the ignition cycle was approximately 210 J.

Deflagration data was acquired using a variable reluctance Validyne DP215 pressure transducer connected to a data acquisition system. A gas mixture was considered to have deflagrated if the pressure rise was greater than or equal to 5%.

### V. Results of Flammability Testing

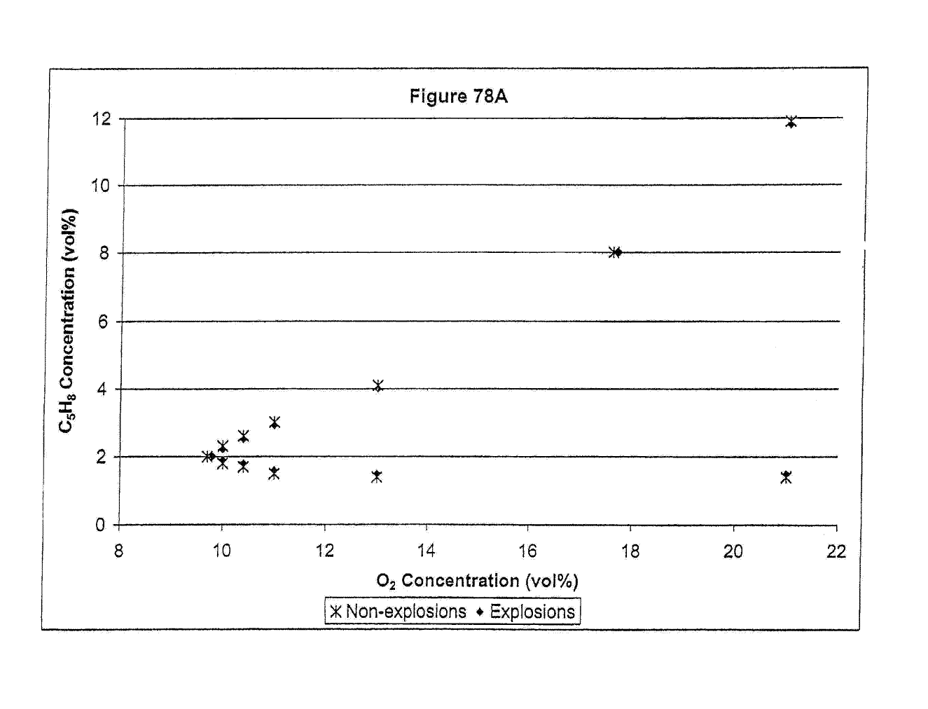

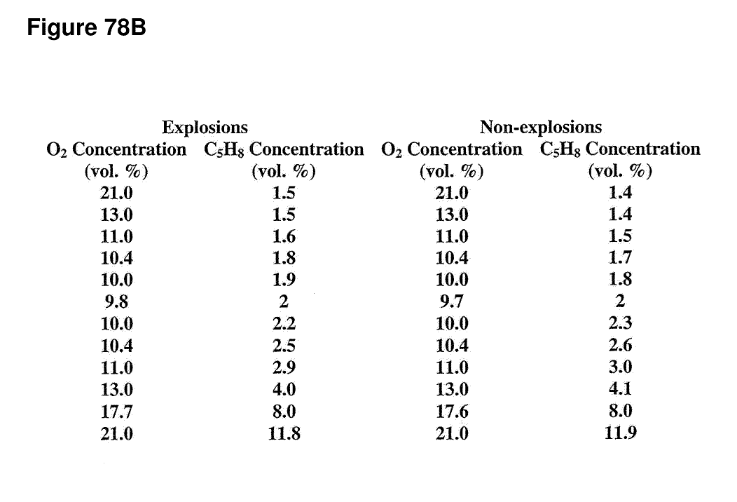

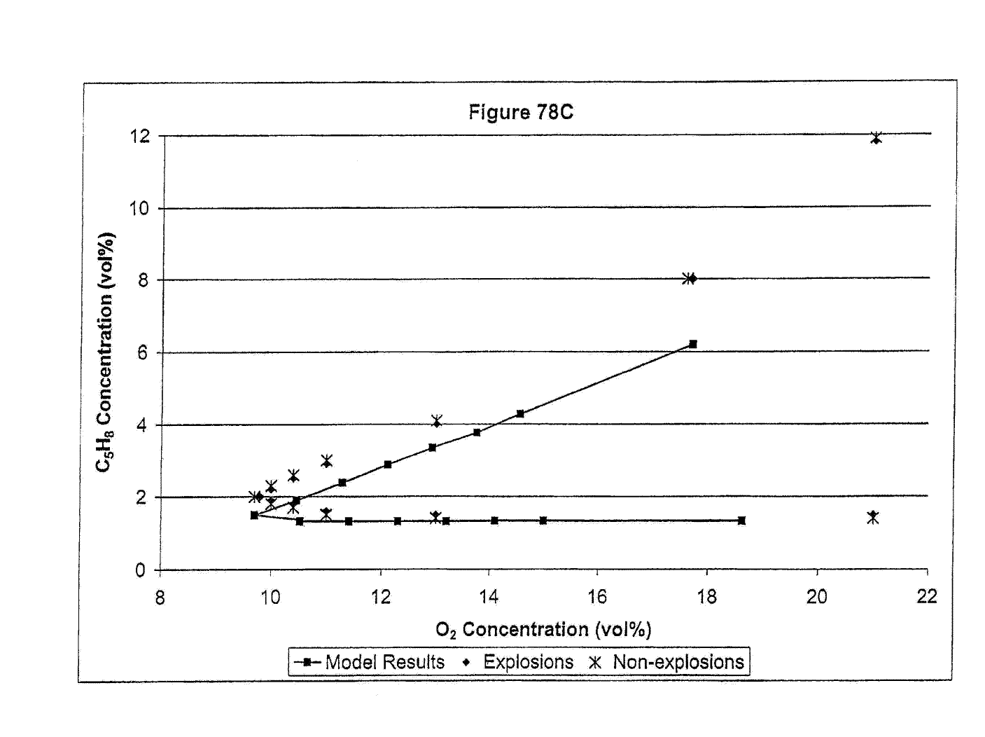

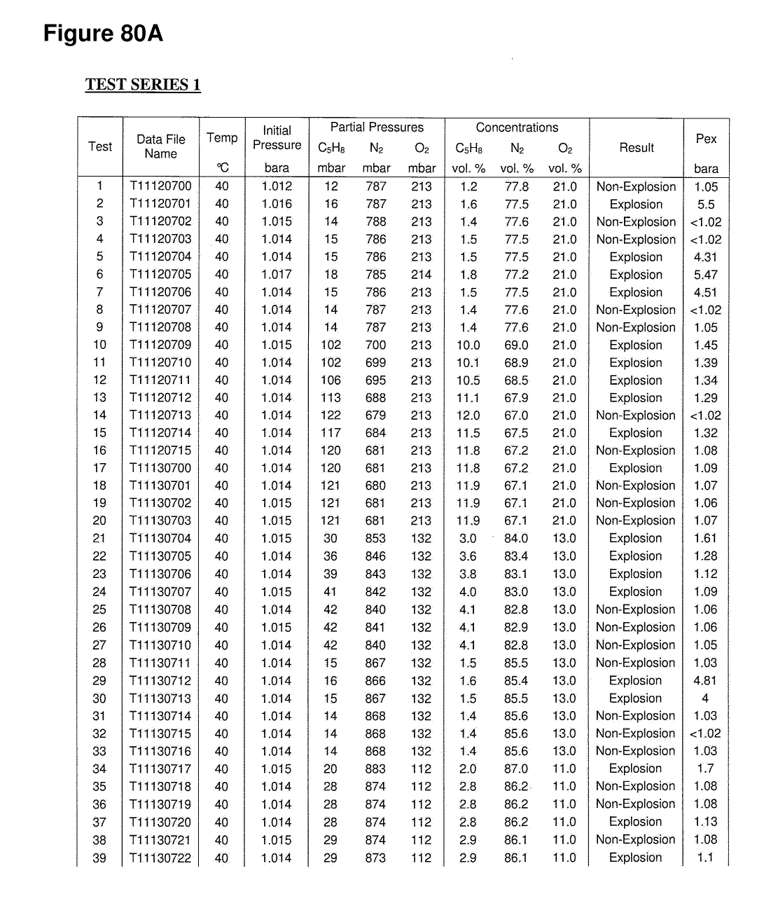

The first experimental series (Series 1) was run at 40° C. and 0 psig with no steam. Running tests at varying concentrations of isoprene and oxygen produced the flammability curve shown in FIG. 78A. The data points shown in this curve are only those that border the curve. A detailed list of all the data points taken for this series is shown in FIGS. 80A and 80B.

FIG. 78B summarizes the explosibility data points shown in FIG. 78A. FIG. 78C is a comparison of the experimental data with the CAFT model predicted flammability envelope. The model agrees very well with the experimental data. Discrepancies may be due to the non-adiabatic nature of the test chamber and limitations of the model. The model looks at an infinite time horizon for the oxidation reaction and does not take into consideration any reaction kinetic limitation.

Additionally, the model is limited by the number of equilibrium chemical species that are in its database and thus may not properly predict pyrolytic species. Also, the flammability envelope developed by the model uses one value for a limit flame temperature (1500K). The limit flame temperature can be a range of values from 1,000K to 1,500K depending on the reacting chemical species. The complex nature of pyrolytic chemical species formed at fuel concentrations above the stoichiometric fuel/oxidizer level is one reason why the model may not accurately predict the upper flammable limit for this system.

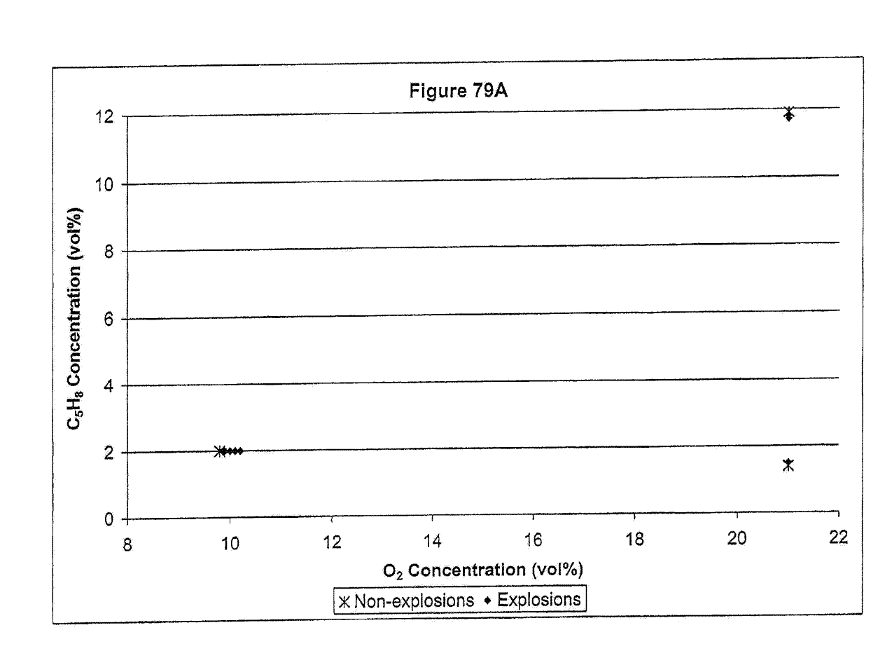

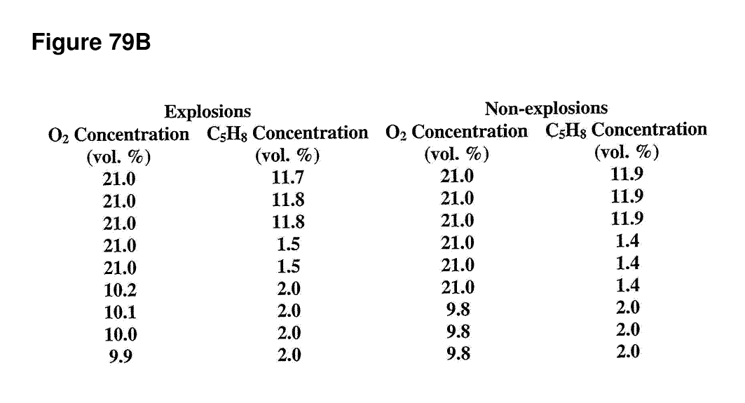

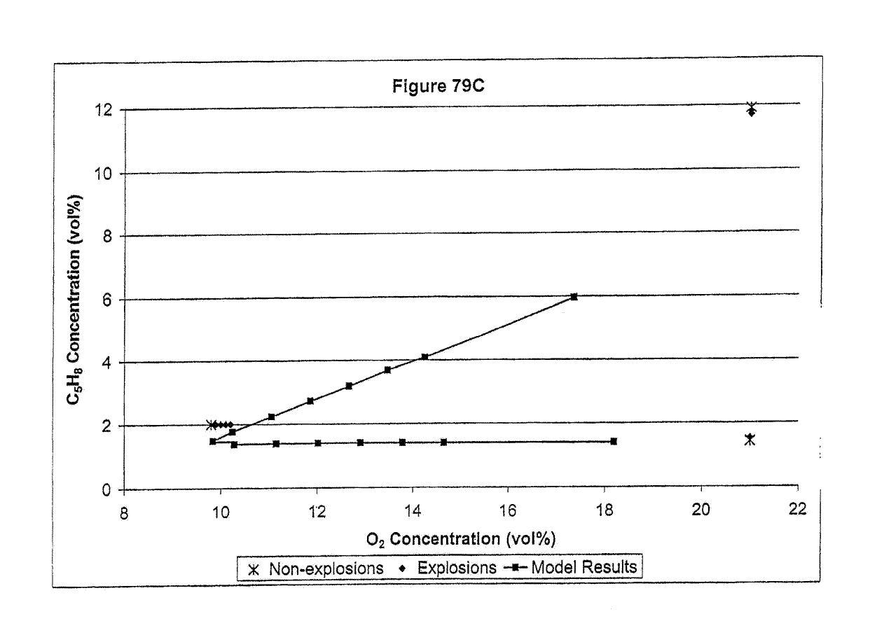

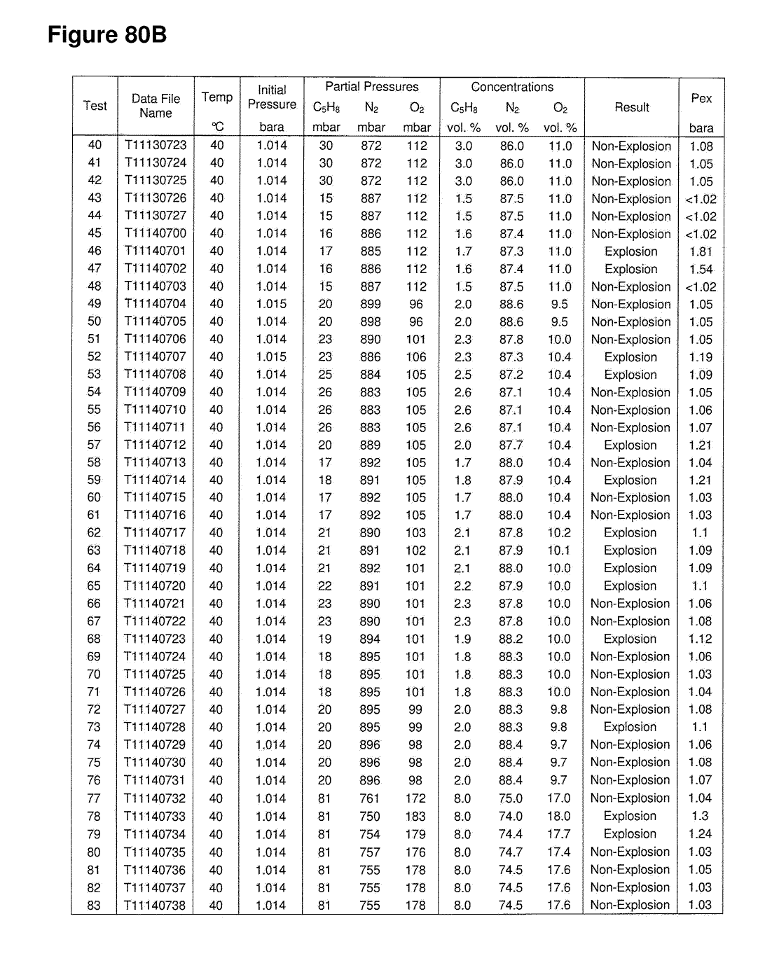

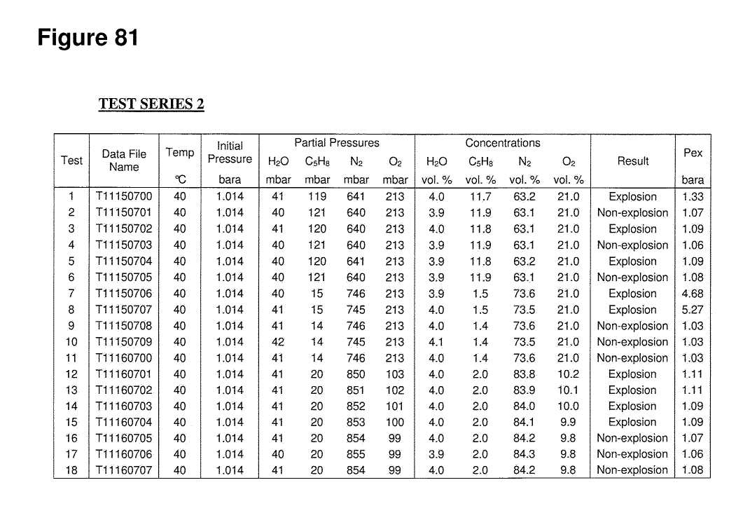

The second experimental series (Series 2) was run at 40° C. and 0 psig with a fixed steam concentration of 4%. Running tests at varying concentrations of isoprene and oxygen produced the flammability curve shown in FIG. 79A. The data points shown in this curve are only those that border the curve. A detailed list of all the data points taken for this series is shown in FIG. 81. Due to the similarity between the data in Series 1 only the key points of lower flammable limit, limiting oxygen concentration, and upper flammable limits were tested. The addition of 4% steam to the test mixture did not significantly change the key limits of the flammability envelope. It should be noted that higher concentrations of steam/water and or other inertants may influence the flammability envelope.

FIG. 79B summarizes the explosibility data points shown in FIG. 79A. FIG. 79C is a comparison of the experimental data with the CAFT model predicted flammability envelope. The model agrees very well with the experimental data. Discrepancies may be due to the same factors described in Series 1

### V. Calculation of Flammability Limits of Isoprene in Air at 3 Atmospheres of Pressure

The methods described in Example 24, parts I to IV were also used to calculate the flammability limits of isoprene at an absolute system pressure of 3 atmospheres and 40° C. These results were compared to those of Example 24, parts I to IV at an absolute system pressure of 1 atmosphere and 40° C. This higher pressure was tested because the flammability envelope expands or grows larger as the initial system pressure is increased. The upper flammability limit is affected the most, followed by the limiting oxygen composition. The lower flammability limit is the least affected (see, for example, “Bulletin 627—Flammability Characteristics of Combustible Gases and Vapors” written by Michael G. Zabetakis and published by the former US Bureau of Mines (1965), which is hereby incorporated by reference in its entirety, particular with respect to the calculation of flammability limits).

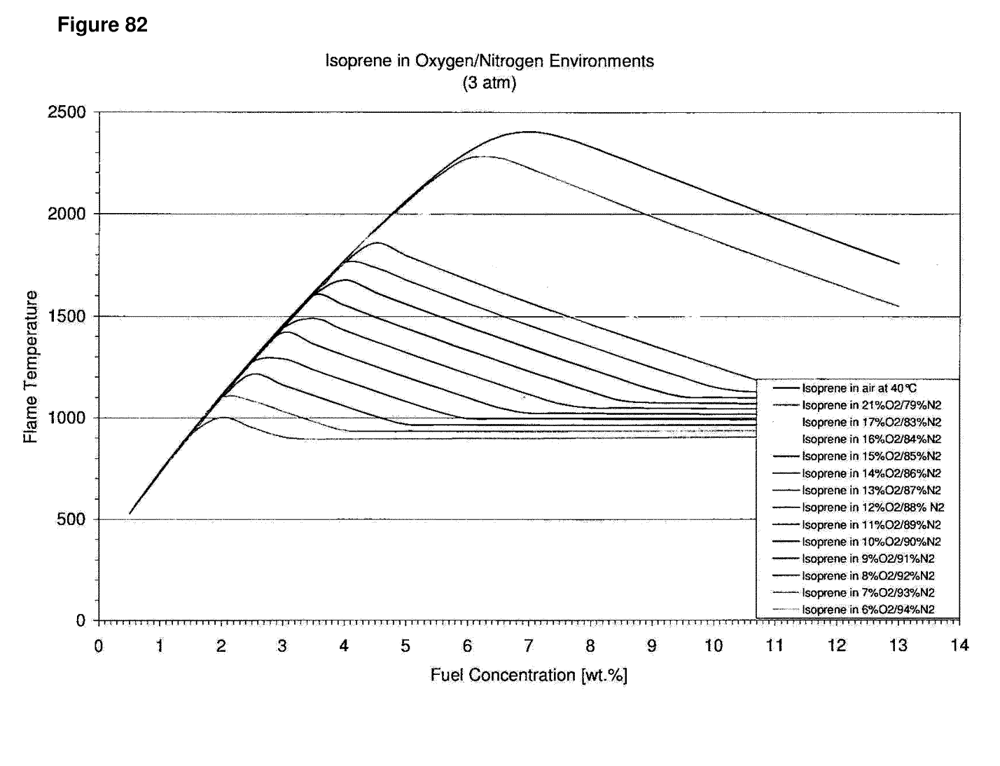

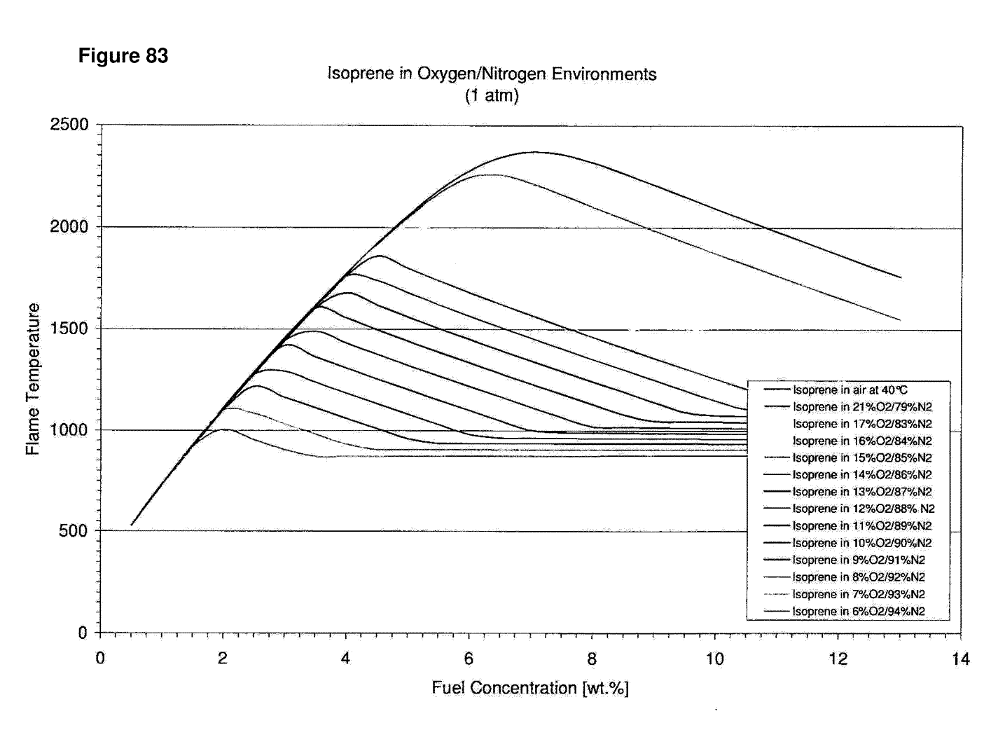

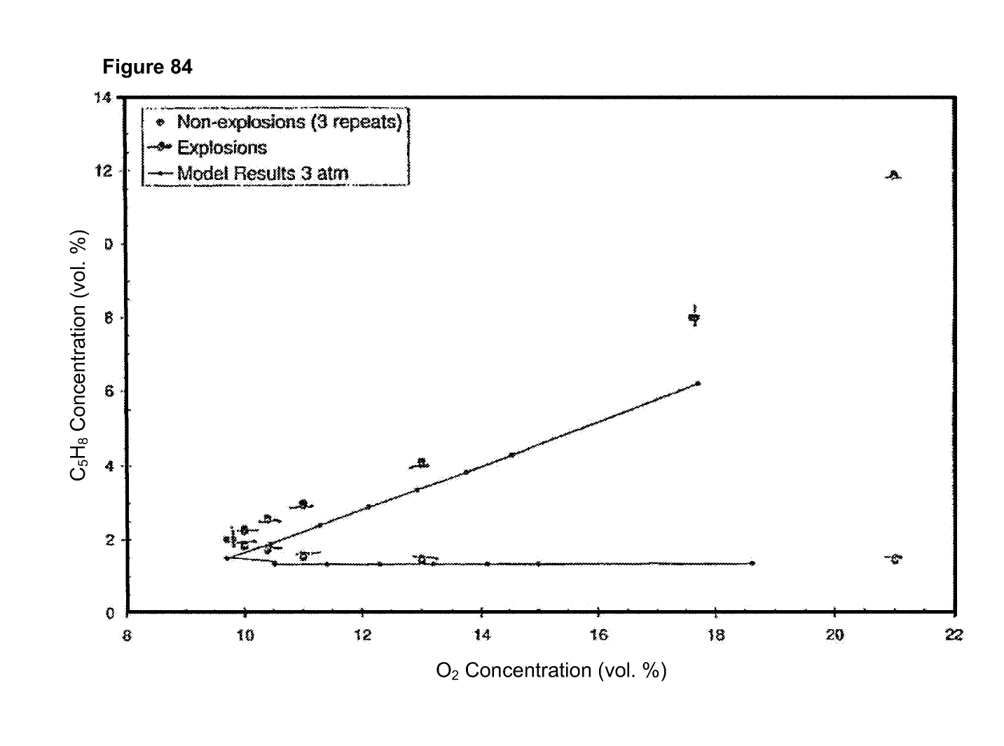

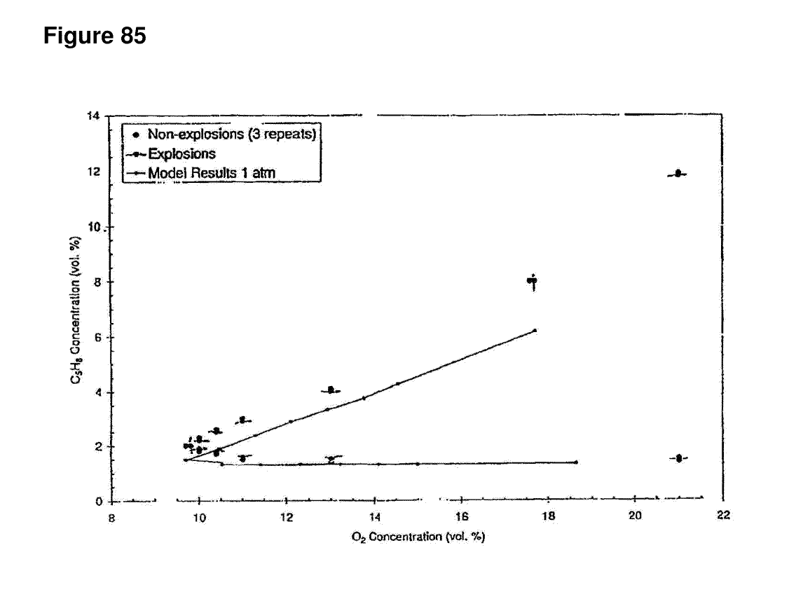

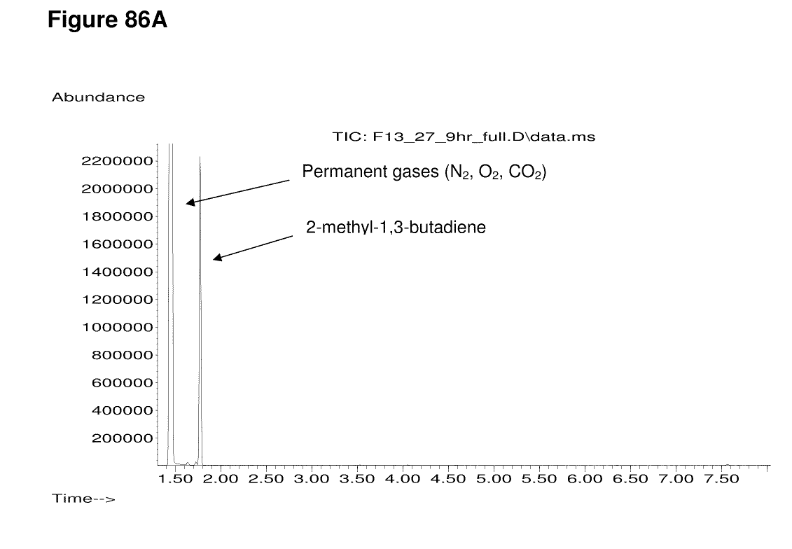

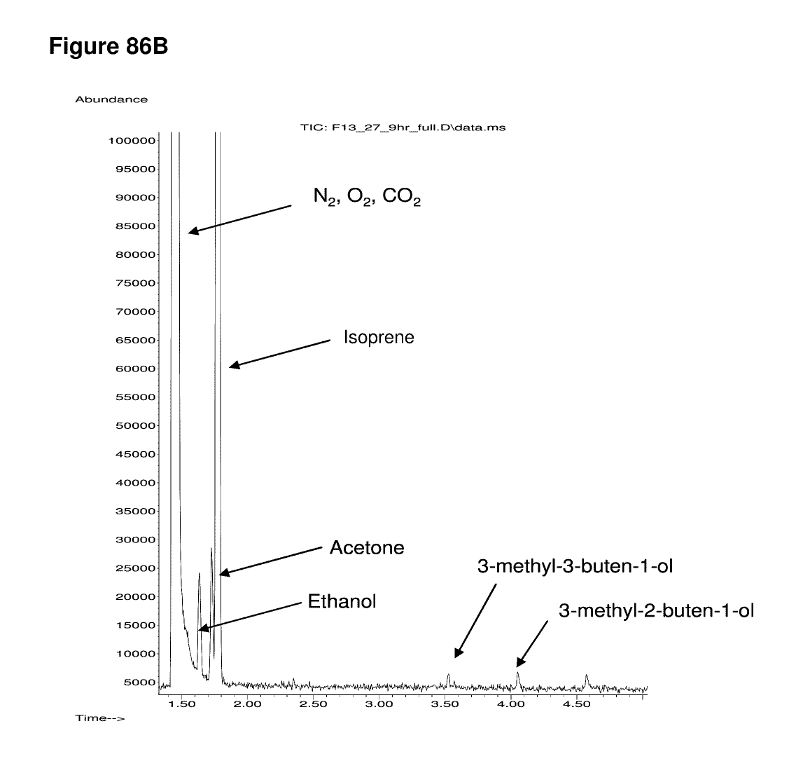

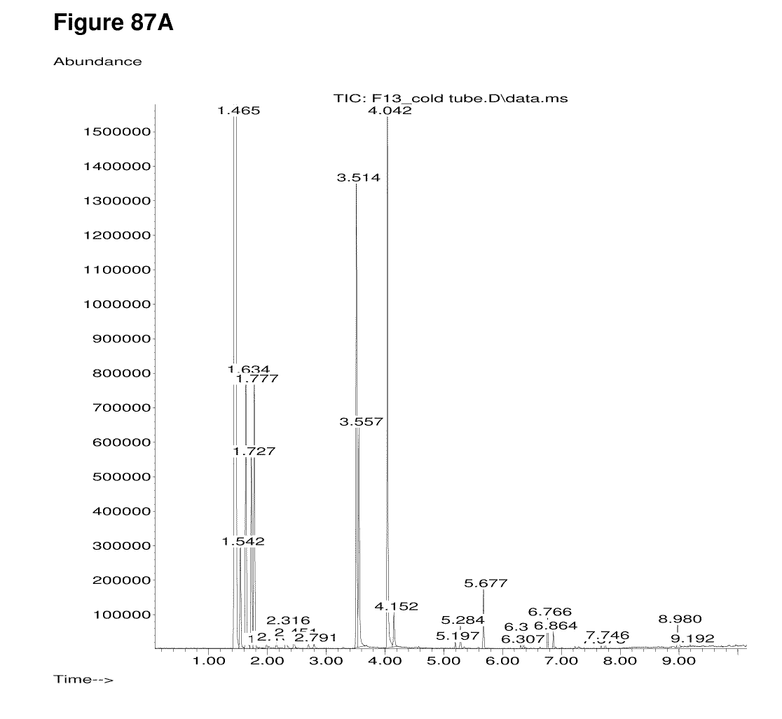

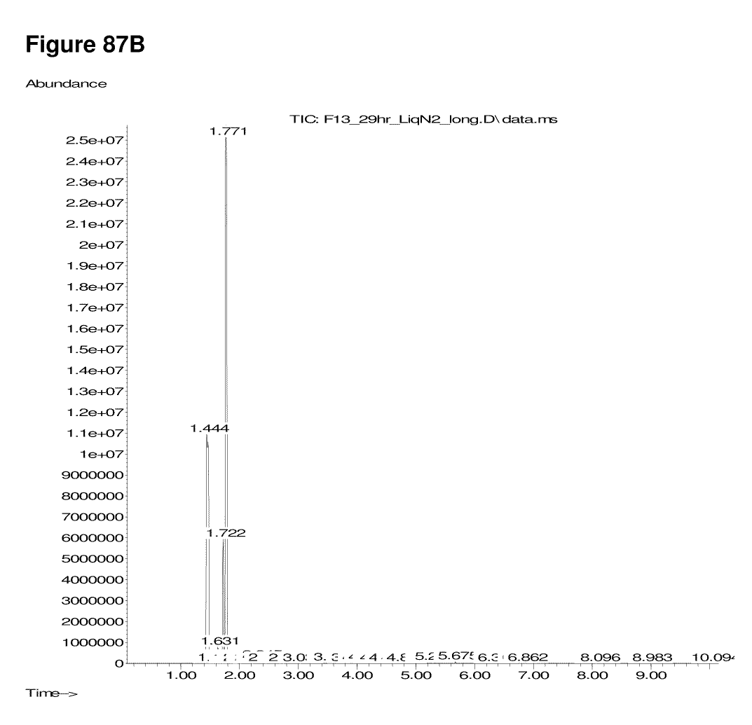

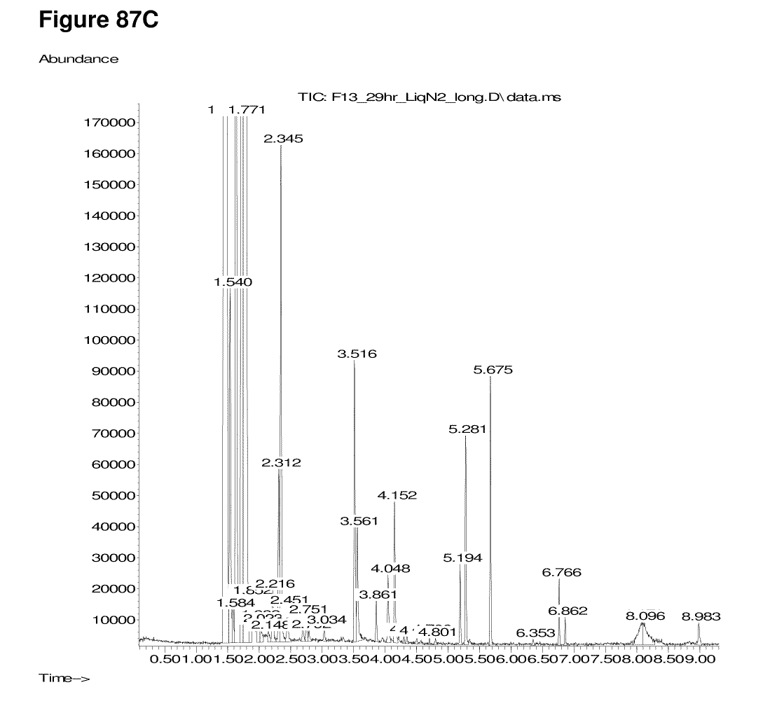

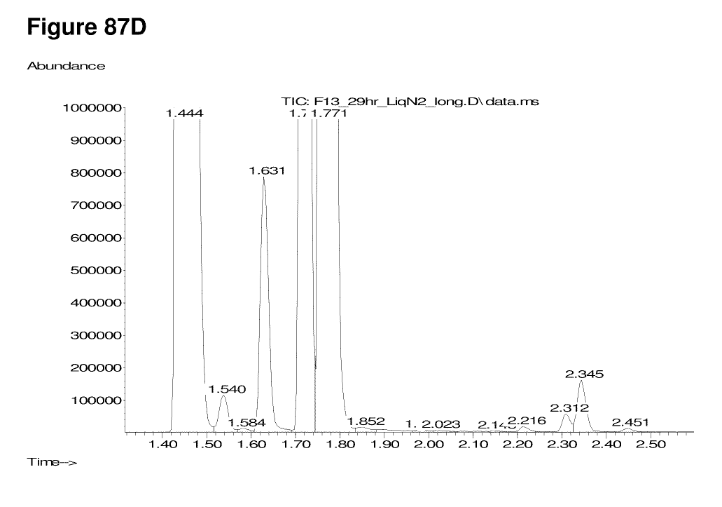

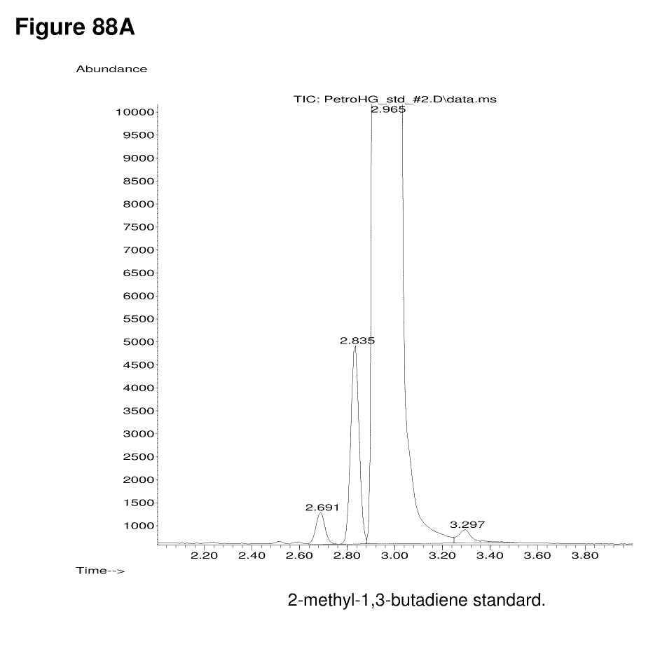

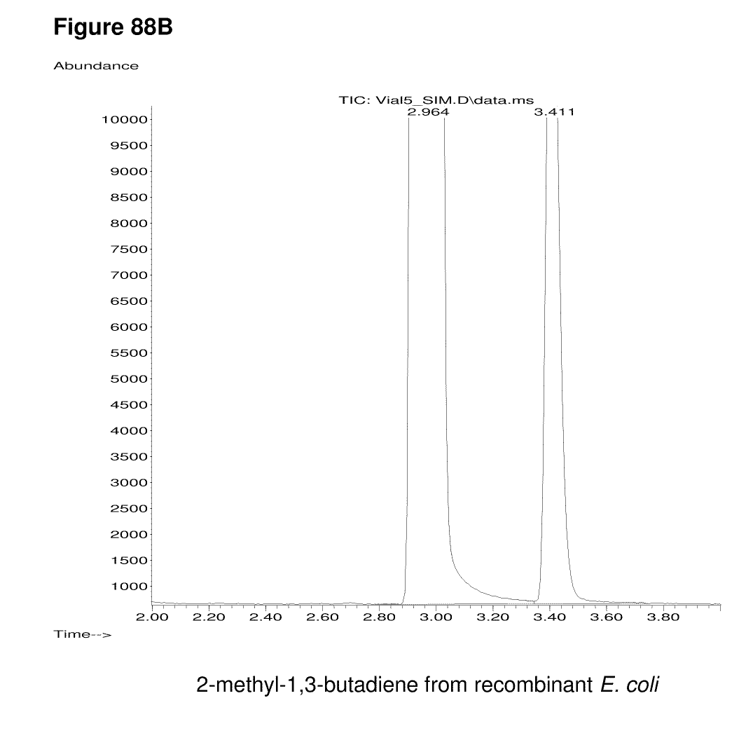

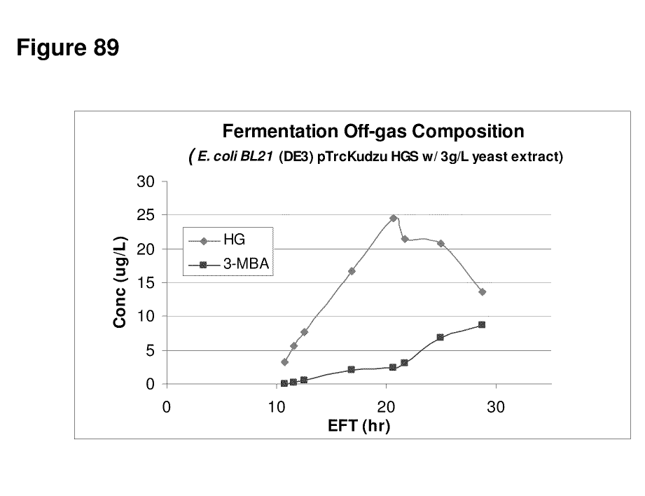

In FIG. 82, the calculated adiabatic flame temperature is plotted as a function of isoprene (fuel) concentration, expressed in weight percent of the total fuel/nitrogen/oxygen, where the system pressure was initially 3 atmospheres. The calculated flame temperatures are very similar to those determined initially in the 1 atmosphere system (FIG. 83). As a result, when flammability envelopes are generated using the calculated adiabatic flammability data, the curves are very similar (see FIGS. 84 and 85). Therefore, based on these theoretical calculations, a system pressure increase from 1 atmosphere to 3 atmosphere does not result in a significant increase/broadening of the flammability envelope. If desired, these model results may be validated using experimental testing (such as the experimental testing described herein at a pressure of 1 atmosphere).

### VII. Summary of Flammability Studies

A calculated adiabatic temperature model was developed for the flammability envelope of the isoprene/oxygen/nitrogen/water/carbon dioxide system at 40° C. and 0 psig. The CAFT model that was developed agreed well with the experimental data generated by the tests conducted in this work. The experimental results from Series 1 and 2 validated the model results from Series A and B.

Unless defined otherwise, the meanings of all technical and scientific terms used herein are those commonly understood by one of skill in the art to which this invention belongs. Singleton, et al., Dictionary of Microbiology and Molecular Biology, 2nd ed., John Wiley and Sons, New York (1994), and Hale & Marham, The Harper Collins Dictionary of Biology, Harper Perennial, N.Y. (1991) provide one of skill with a general dictionary of many of the terms used in this invention. It is to be understood that this invention is not limited to the particular methodology, protocols, and reagents described, as these may vary. One of skill in the art will also appreciate that any methods and materials similar or equivalent to those described herein can also be used to practice or test the invention.

The headings provided herein are not limitations of the various aspects or embodiments of the invention which can be had by reference to the specification as a whole.

For use herein, unless clearly indicated otherwise, use of the terms “a”, “an,” and the like refers to one or more.

Reference to “about” a value or parameter herein includes (and describes) embodiments that are directed to that value or parameter per se. For example, description referring to “about X” includes description of “X.” Numeric ranges are inclusive of the numbers defining the range.

It is understood that aspects and embodiments of the invention described herein include “comprising,” “consisting,” and “consisting essentially of” aspects and embodiments.

### Appendix 1

Exemplary 1-deoxy-D-xylulose-5-phosphate Synthase Nucleic Acids and Polypeptides
ATH: AT3G21500(DXPS1) AT4G15560(CLA1) AT5G11380(DXPS3)
OSA: 4338768 4340090 4342614
CME: CMF089C
PFA: MAL13P1.186
TAN: TA20470
TPV: TP01_{—}0516
ECO: b0420(dxs)
ECJ: JW0410(dxs)
ECE: Z0523(dxs)
ECS: ECs0474
ECC: c0531(dxs)
ECI: UTI89_C0443(dxs)
ECP: ECP_{—}0479
ECV: APECO1_{—}1590(dxs)
ECW: EcE24377A_{—}0451(dxs)
ECX: EcHS_A0491
STY: STY0461(dxs)
STT: t2441(dxs)
SPT: SPA2301(dxs)
SEC: SC0463(dxs)
STM: STM0422(dxs)
YPE: YP03177(dxs)
YPK: y1008(dxs)
YPM: YP_{—}0754(dxs)
YPA: YPA_{—}2671
YPN: YPN 0911
YPP: YPDSF_{—}2812
YPS: YPTB0939(dxs)
YPI: YpsIP31758_{—}3112(dxs)
SFL: SF0357(dxs)
SFX: S0365(dxs)
SFV: SFV_{—}0385(dxs)
SSN: SSON_{—}0397(dxs)
SBO: SBO_{—}0314(dxs)
SDY: SDY_{—}0310(dxs)
ECA: ECA1131(dxs)
PLU: plu3887(dxs)
BUC: BU464(dxs)
BAS: BUsg448(dxs)
WBR: WGLp144(dxs)
SGL: SG0656
KPN: KPN_{—}00372(dxs)
BFL: Bfl238(dxs)
BPN: BPEN_{—}244(dxs)
HIN: HI1439(dxs)
HIT: NTHI1691(dxs)
HIP: CGSHiEE_{—}04795
HIQ: CGSHiGG_{—}01080
HDU: HD0441(dxs)
HSO: HS_{—}0905(dxs)
PMU: PM0532(dxs)
MSU: MS1059(dxs)
APL: APL_{—}0207(dxs)
XFA: XF2249
XFT: PD1293(dxs)
XCC: XCC2434(dxs)
XCB: XC_{—}1678
XCV: XCV2764(dxs)
XAC: XAC2565(dxs)
XOO: XOO2017(dxs)
XOM: XOO_{—}1900(XOO1900)
VCH: VC0889
VVU: VV1_{—}0315
VVY: VV0868
VPA: VP0686
VFI: VF0711
PPR: PBPRA0805
PAE: PA4044(dxs)
PAU: PA14_{—}11550(dxs)
PAP: PSPA7_{—}1057(dxs)
PPU: PP_{—}0527(dxs)
PST: PSPTO_{—}0698(dxs)
PSB: Psyr_{—}0604
PSP: PSPPH_{—}0599(dxs)
PFL: PFL_{—}5510(dxs)
PFO: Pfl_{—}5007
PEN: PSEEN0600(dxs)
PMY: Pmen_{—}3844
PAR: Psyc_{—}0221(dxs)
PCR: Pcryo_{—}0245
ACI: ACIAD3247(dxs)
SON: SO_{—}1525(dxs)
SDN: Sden_{—}2571
SFR: Sfri_{—}2790
SAZ: Sama_{—}2436
SBL: Sbal_{—}1357
SLO: Shew_{—}2771
SHE: Shewmr4_{—}2731
SHM: Shewmr7_{—}2804
SHN: Shewana3_{—}2901
SHW: Sputw3181_{—}2831
ILO: IL2138(dxs)
CPS: CPS_{—}1088(dxs)
PHA: PSHAa2366(dxs)
PAT: Patl_{—}1319
SDE: Sde_{—}3381
PIN: Ping_{—}2240
MAQ: Maqu_{—}2438
MCA: MCA0817(dxs)
FTU: FTT1018c(dxs)
FTF: FTF1018c(dxs)
FTW: FTW_{—}0925(dxs)
FTL: FTL_{—}1072
FTH: FTH_{—}1047(dxs)
FTA: FTA_{—}1131(dxs)
FTN: FTN_{—}0896(dxs)
NOC: Noc_{—}1743
AEH: Mlg_{—}1381
HCH: HCH_{—}05866(dxs)
CSA: Csal_{—}0099
ABO: ABO_{—}2166(dxs)
AHA: AHA_{—}3321(dxs)
BCI: BCI_{—}0275(dxs)
RMA: Rmag_{—}0386
VOK: COSY_{—}0360(dxs)
NME: NMB1867
NMA: NMA0589(dxs)
NMC: NMCO352(dxs)
NGO: NG00036
CVI: CV_{—}2692(dxs)
RSO: RSc2221(dxs)
REU: Reut_A0882
REH: H16_A2732(dxs)
RME: Rmet_{—}2615
BMA: BMAA0330(dxs)
BMV: BMASAVP1_{—}1512(dxs)
BML: BMA10299_{—}1706(dxs)
BMN: BMA10247_A0364(dxs)
BXE: Bxe_B2827
BUR: Bcep18194_B2211
BCN: Bcen_{—}4486
BCH: Bcen2424_{—}3879
BAM: Bamb_{—}3250
BPS: BPSS1762(dxs)
BPM: BURPS1710b_A0842(dxs)
BPL: BURPS1106A_A2392(dxs)
BPD: BURPS668_A2534(dxs)
BTE: BTH_II0614(dxs)
BPE: BP2798(dxs)
BPA: BPP2464(dxs)
BBR: BB1912(dxs)
RFR: Rfer_{—}2875
POL: Bpro_{—}1747
PNA: Pnap_{—}1501
AJS: Ajs_{—}1038
MPT: Mpe_A2631
HAR: HEAR0279(dxs)
MMS: mma_{—}0331
NEU: NE1161(dxs)
NET: Neut_{—}1501
NMU: Nmul_A0236
EBA: ebA4439(dxs)
AZO: azo1198(dxs)
DAR: Daro_{—}3061
TBD: Tbd_{—}0879
MFA: Mfla_{—}2133
HPY: HP0354(dxs)
HPJ: jhp0328(dxs)
HPA: HPAG1_{—}0349
HHE: HH0608(dxs)
HAC: Hac_{—}0968(dxs)
WSU: WS1996
TDN: Tmden_{—}0475
CJE: Cj0321(dxs)
CJR: CJE0366(dxs)
CJJ: CJJ81176_{—}0343(dxs)
CJU: C8J_{—}0298(dxs)
CJD: JJD26997_{—}1642(dxs)
CFF: CFF8240_{—}0264(dxs)
CCV: CCV52592_{—}1671(dxs) CCV52592_{—}1722
CHA: CHAB381_{—}1297(dxs)
CCO: CCC13826_{—}1594(dxs)
ABU: Abu_{—}2139(dxs)
NIS: NIS_{—}0391(dxs)
SUN: SUN_{—}2055(dxs)
GSU: GSU0686(dxs-1) GSU1764(dxs-2)
GME: Gmet_{—}1934 Gmet_{—}2822
PCA: Pcar_{—}1667
PPD: Ppro_{—}1191 Ppro_{—}2403
DVU: DVU1350(dxs)
DVL: Dvul_{—}1718
DDE: Dde_{—}2200
LIP: LI0408(dsx)
DPS: DP2700
ADE: Adeh_{—}1097
MXA: MXAN_{—}4643(dxs)
SAT: SYN_{—}02456
SFU: Sfum_{—}1418
PUB: SAR11_{—}0611(dxs)
MLO: mlr7474
MES: Meso_{—}0735
SME: SMc00972(dxs)
ATU: Atu0745(dxs)
ATC: AGR_C_{—}1351
RET: RHE_CH00913(dxs)
RLE: RL0973(dxs)
BME: BMEI1498
BMF: BAB1_{—}0462(dxs)
BMS: BR0436(dxs)
BMB: BruAb1_{—}0458(dxs)
BOV: BOV_{—}0443(dxs)
BJA: bll2651(dxs)
BRA: BRADO2161(dxs)
BBT: BBta_{—}2479(dxs)
RPA: RPA0952(dxs)
RPB: RPB_{—}4460
RPC: RPC 1149
RPD: RPD_{—}4305
RPE: RPE_{—}1067
NWI: Nwi_{—}0633
NHA: Nham_{—}0778
BHE: BH04350(dxs)
BQU: BQ03540(dxs)
BBK: BARBAKC583_{—}0400(dxs)
CCR: CC_{—}2068
SIL: SP00247(dxs)
SIT: TM1040_{—}2920
RSP: RSP_{—}0254(dxsA) RSP_{—}1134(dxs)
JAN: Jann_{—}0088 Jann_{—}0170
RDE: RD1_{—}0101(dxs) RD1_{—}0548(dxs)
MMR: Mmar10_{—}0849
HNE: HNE_{—}1838(dxs)
ZMO: ZMO1234(dxs) ZMO1598(dxs)
NAR: Saro_{—}0161
SAL: Sala_{—}2354
ELI: ELI_{—}12520
GOX: GOX0252
GBE: GbCGDNIH1_{—}0221 GbCGDNIH1_{—}2404
RRU: Rru_A0054 Rru_A2619
MAG: amb2904
MGM: Mmc1_{—}1048
SUS: Acid_{—}1783
BSU: BG11715(dxs)
BHA: BH2779
BAN: BA4400(dxs)
BAR: GBAA4400(dxs)
BAA: BA_{—}4853
BAT: BAS4081
BCE: BC4176(dxs)
BCA: BCE_{—}4249(dxs)
BCZ: BCZK3930(dxs)
BTK: BT9727_{—}3919(dxs)
BTL: BALH_{—}3785(dxs)
BLI: BL01523(dxs)
BLD: BLi02598(dxs)
BCL: ABC2462(dxs)
BAY: RBAM_{—}022600
BPU: BPUM_{—}2159
GKA: GK2392
GTN: GTNG_{—}2322
LMO: lmo1365(tktB)
LMF: LMOf2365_{—}1382(dxs)
LIN: lin1402(tktB)
LWE: lwe1380(tktB)
LLA: L108911(dxsA) L123365(dxsB)
LLC: LACR_{—}1572 LACR_{—}1843
LLM: llmg_{—}0749(dxsB)
SAK: SAK_{—}0263
LPL: lp_{—}2610(dxs)
LJO: LJ0406
LAC: LBA0356
LSL: LSL_{—}0209(dxs)
LGA: LGAS_{—}0350
STH: STH1842
CAC: CAC2077 CA_P0106(dxs)
CPE: CPE1819
CPF: CPF_{—}2073(dxs)
CPR: CPR_{—}1787(dxs)
CTC: CTC01575
CNO: NT01CX_{—}1983
CTH: Cthe_{—}0828
CDF: CD1207(dxs)
CBO: CBO1881(dxs)
CBA: CLB_{—}1818(dxs)
CBH: CLC_{—}1825(dxs)
CBF: CLI_{—}1945(dxs)
CKL: CKL_{—}1231(dxs)
CHY: CHY_{—}1985(dxs)
DSY: DSY2348
DRM: Dred_{—}1078
PTH: PTH_{—}1196(dxs)
SWO: Swol_{—}0582
CSC: Csac_{—}1853
TTE: TTE1298(dxs)
MTA: Moth_{—}1511
MPE: MYPE730
MGA: MGA_{—}1268(dxs)
MTU: Rv2682c(dxs1) Rv3379c(dxs2)
MTC: MT2756(dxs)
MBO: Mb2701c(dxs1) Mb3413c(dxs2)
MLE: ML1038(dxs)
MPA: MAP2803c(dxs)
MAV: MAV_{—}3577(dxs)
MSM: MSMEG_{—}2776(dxs)
MMC: Mmcs_{—}2208
CGL: NCgl1827(cgl1902)
CGB: cg2083(dxs)
CEF: CE1796
CDI: DIP1397(dxs)
CJK: jk1078(dxs)
NFA: nfa37410(dxs)
RHA: RHA1_ro06843
SCO: SCO6013(SC1C3.01) SCO6768(SC6A5.17)
SMA: SAV1646(dxs1) SAV2244(dxs2)
TWH: TWT484
TWS: TW280(Dxs)
LXX: Lxx10450(dxs)
CMI: CMM_{—}1660(dxsA)
AAU: AAur_{—}1790(dxs)
PAC: PPA1062
TFU: Tfu_{—}1917
FRA: Francci3_{—}1326
FAL: FRAAL2088(dxs)
ACE: Ace1_{—}1393
SEN: SACE_{—}1815(dxs) SACE_{—}4351
BLO: BL1132(dxs)
BAD: BAD_{—}0513(dxs)
FNU: FN1208 FN1464
RBA: RB2143(dxs)
CTR: CT331(dxs)
CTA: CTA_{—}0359(dxs)
CMU: TC0608
CPN: CPn1060(tktB_{—}2)
CPA: CP0790
CPJ: CPj1060(tktB_{—}2)
CPT: CpB1102
CCA: CCA00304(dxs)
CAB: CAB301(dxs)
CFE: CF0699(dxs)
PCU: pc0619(dxs)
TPA: TP0824
TDE: TDE1910(dxs)
LIL: LA3285(dxs)
LIC: LIC10863(dxs)
LBJ: LBJ_{—}0917(dxs)
LBL: LBL_{—}0932(dxs)
SYN: sll1945(dxs)
SYW: SYNW1292(Dxs)
SYC: syc1087_c(dxs)
SYF: Synpcc7942_{—}0430
SYD: Syncc9605_{—}1430
SYE: Syncc9902_{—}1069
SYG: sync_{—}1410(dxs)
SYR: SynRCC307_{—}1390(dxs)
SYX: SynWH7803_{—}1223(dxs)
CYA: CYA_{—}1701(dxs)
CYB: CYB_{—}1983(dxs)
TEL: tll0623
GVI: gll0194
ANA: alr0599
AVA: Ava_{—}4532
PMA: Pro0928(dxs)
PMM: PMM0907(Dxs)
PMT: PMT0685(dxs)
PMN: PMN2A_{—}0300
PMI: PMT9312_{—}0893
PMB: A9601_{—}09541(dxs)
PMC: P9515_{—}09901(dxs)
PMF: P9303_{—}15371(dxs)
PMG: P9301_{—}09521(dxs)
PMH: P9215_{—}09851
PMJ: P9211_{—}08521
PME: NATL1_{—}09721(dxs)
TER: Tery_{—}3042
BTH: BT_{—}1403 BT_{—}4099
BFR: BF0873 BF4306
BFS: BF0796(dxs) BF4114
PGI: PG2217(dxs)
CHU: CHU_{—}3643(dxs)
GFO: GFO_{—}3470(dxs)
FPS: FP0279(dxs)
CTE: CT0337(dxs)
CPH: Cpha266_{—}0671
PVI: Cvib_{—}0498
PLT: Plut_{—}0450
DET: DET0745(dxs)
DEH: cbdb_A720(dxs)
DRA: DR_{—}1475
DGE: Dgeo_{—}0994
TTH: TTC1614
TTJ: TTHA0006
AAE: aq_{—}881
TMA: TM1770
PMO: Pmob_{—}1001

### Exemplary Acetyl-CoA-acetyltransferase Nucleic Acids and Polypeptides

HSA: 38(ACAT1) 39(ACAT2)
PTR: 451528(ACAT1)
MCC: 707653(ACAT1) 708750(ACAT2)
MMU: 110446(Acat1) 110460(Acat2)
RNO: 25014(Acat1)
CFA: 484063(ACAT2) 489421(ACAT1)
GGA: 418968(ACAT1) 421587(RCJMB04_{—}34i5)
XLA: 379569(MGC69098) 414622(MGC81403) 414639(MGC81256) 444457(MGC83664)
XTR: 394562(acat2)
DRE: 30643(acat2)
SPU: 759502(LOC759502)
DME: Dmel_CG10932 Dmel_CG9149
CEL: T02G5.4 T02G5.7 T02G5.8(kat-1)
ATH: AT5G48230(ACAT2/EMB1276)
OSA: 4326136 4346520
CME: CMA042C CME087C
SCE: YPL028W(ERG10)
AGO: AGOS_ADR165C
PIC: PICST_{—}31707(ERG10)
CAL: Ca019.1591(erg10)
CGR: CAGL0L12364g
SPO: SPBC215.09c
MGR: MGG_{—}01755 MGG_{—}13499
ANI: AN1409.2
AFM: AFUA_{—}6G14200 AFUA_{—}8G04000
AOR: AO090103000012 AO090103000406
CNE: CNC05280
UMA: UM03571.1
DDI: DDB_{—}0231621
PFA: PF14_{—}0484
TET: TTHERM_{—}00091590 TTHERM_{—}00277470 TTHERM_{—}00926980
TCR: 511003.60
ECO: b2224(atoB)
ECJ: JW2218(atoB) JW5453(yqeF)
ECE: Z4164(yqeF)
ECS: ECs3701
ECC: c2767(atoB) c3441(yqeF)
ECI: UTI89_C2506(atoB) UTI89_C3247(yqeF)
ECP: ECP_{—}2268 ECP_{—}2857
ECV: APECO1_{—}3662(yqeF) APECO1_{—}4335(atoB) APECO1_{—}43352(atoB)
ECX: EcHS_A2365
STY: STY3164(yqeF)
STT: t2929(yqeF)
SPT: SPA2886(yqeF)
SEC: SC2958(yqeF)
STM: STM3019(yqeF)
SFL: SF2854(yqeF)
SFX: 53052(yqeF)
SFV: SFV_{—}2922(yqeF)
SSN: SSON_{—}2283(atoB) SSON_{—}3004(yqeF)
SBO: SBO_{—}2736(yqeF)
ECA: ECA1282(atoB)
ENT: Ent638_{—}3299
SPE: Spro_{—}0592
HIT: NTHI0932(atoB)
XCC: XCC1297(atoB)
XCB: XC_{—}2943
XCV: XCV1401(thlA)
XAC: XAC1348(atoB)
XOO: XOO1881(atoB)
XOM: XOO_{—}1778(XOO1778)
VCH: VCA0690
VCO: VCO395_{—}0630
VVU: VV2_{—}0494 VV2_{—}0741
VVY: VVA1043 VVA1210
VPA: VPA0620 VPA1123 VPA1204
PPR: PBPRB1112 PBPRB1840
PAE: PA2001(atoB) PA2553 PA3454 PA3589 PA3925
PAU: PA14_{—}38630(atoB)
PPU: PP_{—}2051(atoB) PP_{—}2215(fadAx) PP_{—}3754 PP_{—}4636
PPF: Pput_{—}2009 Pput_{—}2403 Pput_{—}3523 Pput_{—}4498
PST: PSPTO_{—}0957(phbA-1) PSPTO_{—}3164(phbA-2)
PSB: Psyr_{—}0824 Psyr_{—}3031
PSP: PSPPH_{—}0850(phbA1) PSPPH_{—}2209(phbA2)
PFL: PFL_{—}1478(atoB-2) PFL_{—}2321 PFL_{—}3066 PFL_{—}4330(atoB-2) PFL_{—}5283
PFO: Pfl_{—}1269 Pfl_{—}1739 Pfl_{—}2074 Pfl_{—}2868
PEN: PSEEN3197 PSEEN3547(fadAx) PSEEN4635(phbA)
PMY: Pmen_{—}1138 Pmen_{—}2036 Pmen_{—}3597 Pmen_{—}3662 Pmen_{—}3820
PAR: Psyc_{—}0252 Psyc_{—}1169
PCR: Pcryo_{—}0278 Pcryo_{—}1236 Pcryo_{—}1260
PRW: PsycPRwf_{—}2011
ACI: ACIAD0694 ACIAD1612 ACIAD2516(atoB)
SON: SO_{—}1677(atoB)
SDN: Sden_{—}1943
SFR: Sfri_{—}1338 Sfri_{—}2063
SAZ: Sama_{—}1375
SBL: Sbal_{—}1495
SBM: Shew185_{—}1489
SBN: Sbal195_{—}1525
SLO: Shew_{—}1667 Shew_{—}2858
SPC: Sputcn32_{—}1397
SSE: Ssed_{—}1473 Ssed_{—}3533
SPL: Spea_{—}2783
SHE: Shewmr4_{—}2597
SHM: Shewmr7_{—}2664
SHN: Shewana3_{—}2771
SHW: Sputw3181_{—}2704
ILO: IL0872
CPS: CPS_{—}1605 CPS_{—}2626
PHA: PSHAa0908 PSHAa1454(atoB) PSHAa1586(atoB)
PAT: Patl_{—}2923
SDE: Sde_{—}3149
PIN: Ping_{—}0659 Ping_{—}2401
MAQ: Maqu_{—}2117 Maqu_{—}2489 Maqu_{—}2696 Maqu_{—}3162
CBU: CBU_{—}0974
LPN: lpg1825(atoB)
LPF: lpl1789
LPP: lppl1788
NOC: Noc_{—}1891
AEH: Mlg_{—}0688 Mlg_{—}2706
HHA: Hhal_{—}1685
HCH: HCH_{—}05299
CSA: Csal_{—}0301 Csal_{—}3068
ABO: ABO_{—}0648(fadAx)
MMW: Mmwyl1_{—}0073 Mmwyl1_{—}3021 Mmwyl1_{—}3053 Mmwyl1_{—}3097 Mmwyl1_{—}4182
AHA: AHA_{—}2143(atoB)
CVI: CV_{—}2088(atoB) CV_{—}2790(phaA)
RSO: RSc0276(atoB) RSc1632(phbA) RSc1637(bktB) RSc1761(RS02948)
REU: Reut_A0138 Reut_A1348 Reut_A1353 Reut_B4561 Reut_B4738 Reut_B5587 Reut_C5943 Reut_C6062
REH: H16_A0170 H16_A0867 H16_A0868 H16_A0872 H16_A1297 H16_A1438(phaA) H16_A1445(bktB) H16_A1528 H16_A1713 H16_A1720 H16_A1887 H16_A2148 H16_B0380 H16_B0381 H16_B0406 H16_B0662 H16_B0668 H16_B0759 H16_B1369 H16_B1771
RME: Rmet_{—}0106 Rmet_{—}1357 Rmet_{—}1362 Rmet_{—}5156
BMA: BMA1316 BMA1321(phbA) BMA1436
BMV: BMASAVP1_A1805(bktB) BMASAVP1_A1810(phbA)
BML: BMA10299_A0086(phbA) BMA10299_A0091
BMN: BMA10247_{—}1076(bktB) BMA10247_{—}1081(phbA)
BXE: Bxe_A2273 Bxe_A2335 Bxe_A2342 Bxe_A4255 Bxe_B0377 Bxe_B0739 Bxe_C0332 Bxe_C0574 Bxe_C0915
BVI: Bcep1808_{—}0519 Bcep1808_{—}1717 Bcep1808_{—}2877 Bcep1808_{—}3594 Bcep1808_{—}4015 Bcep1808_{—}5507 Bcep1808_{—}5644
BUR: Bcep18194_A3629 Bcep18194_A5080 Bcep18194_A5091 Bcep18194_A6102 Bcep18194_B0263 Bcep18194_B1439 Bcep18194_C6652 Bcep18194_C6802 Bcep18194_C6874 Bcep18194_C7118 Bcep18194_C7151 Bcep18194_C7332
BCN: Bcen_{—}1553 Bcen_{—}1599 Bcen_{—}2158 Bcen_{—}2563 Bcen_{—}2998 Bcen_{—}6289
BCH: Bcen2424_{—}0542 Bcen2424_{—}1790 Bcen2424_{—}2772 Bcen2424_{—}5368 Bcen2424_{—}6232 Bcen2424_{—}6276
BAM: Bamb_{—}0447 Bamb_{—}1728 Bamb_{—}2824 Bamb_{—}4717 Bamb_{—}5771 Bamb_{—}5969
BPS: BPSL1426 BPSL1535(phbA) BPSL1540
BPM: BURPS1710b_{—}2325(bktB) BURPS1710b_{—}2330(phbA) BURPS1710b_{—}2453(atoB-2)
BPL: BURPS1106A_{—}2197(bktB) BURPS1106A_{—}2202(phbA)
BPD: BURPS668_{—}2160(bktB) BURPS668_{—}2165(phbA)
BTE: BTH_I2144 BTH_I2256 BTH_I2261
PNU: Pnuc_{—}0927
BPE: BP0447 BP0668 BP2059
BPA: BPP0608 BPP1744 BPP3805 BPP4216 BPP4361
BBR: BB0614 BB3364 BB4250 BB4804 BB4947
RFR: Rfer_{—}0272 Rfer_{—}1000 Rfer_{—}1871 Rfer_{—}2273 Rfer_{—}2561 Rfer_{—}2594 Rfer_{—}3839
POL: Bpro_{—}1577 Bpro_{—}2140 Bpro_{—}3113 Bpro_{—}4187
PNA: Pnap_{—}0060 Pnap_{—}0458 Pnap_{—}0867 Pnap_{—}1159 Pnap_{—}2136 Pnap_{—}2804
AAV: Aave_{—}0031 Aave_{—}2478 Aave_{—}3944 Aave_{—}4368
AJS: Ajs_{—}0014 Ajs_{—}0124 Ajs_{—}1931 Ajs_{—}2073 Ajs_{—}2317 Ajs_{—}3548 Ajs_{—}3738 Ajs_{—}3776
VEI: Veis_{—}1331 Veis_{—}3818 Veis_{—}4193
DAC: Daci_{—}0025 Daci_{—}0192 Daci_{—}3601 Daci_{—}5988
MPT: Mpe_A1536 Mpe_A1776 Mpe_A1869 Mpe_A3367
HAR: HEAR0577(phbA)
MMS: mma_{—}0555
NEU: NE2262(bktB)
NET: Neut_{—}0610
EBA: ebA5202 p2A409(tioL)
AZO: azo0464(fadA1) azo0469(fadA2) azo2172(th1A)
DAR: Daro_{—}0098 Daro_{—}3022
HPA: HPAG1_{—}0675
HAC: Hac_{—}0958(atoB)
GME: Gmet_{—}1719 Gmet_{—}2074 Gmet_{—}2213 Gmet_{—}2268 Gmet_{—}3302
GUR: Gura_{—}3043
BBA: Bd0404(atoB) Bd2095
DOL: Dole_{—}0671 Dole_{—}1778 Dole_{—}2160 Dole_{—}2187
ADE: Adeh_{—}0062 Adeh_{—}2365
AFW: Anae109_{—}0064 Anae109_{—}1504
MXA: MXAN_{—}3791
SAT: SYN_{—}02642
SFU: Sfum_{—}2280 Sfum_{—}3582
RPR: RP737
RCO: RC1134 RC1135
RFE: RF_{—}0163(paaJ)
RBE: RBE_{—}0139(paaJ)
RAK: A1C_{—}05820
RBO: A1I_{—}07215
RCM: A1E_{—}04760
PUB: SAR11_{—}0428(th1A)
MLO: mlr3847
MES: Meso_{—}3374
PLA: Plav_{—}1573 Plav_{—}2783
SME: SMa1450 SMc03879(phbA)
SMD: Smed_{—}0499 Smed_{—}3117 Smed_{—}5094 Smed_{—}5096
ATU: Atu2769(atoB) Atu3475
ATC: AGR_C_{—}5022(phbA) AGR_L_{—}2713
RET: RHE_CH04018(phbAch) RHE_PC00068(ypc00040) RHE_PF00014(phbAf)
RLE: RL4621(phaA) pRL100301 pRL120369
BME: BMEI0274 BMEI10817
BMF: BAB1_{—}1783(phbA-1) BAB2_{—}0790(phbA-2)
BMS: BR1772(phbA-1) BRA0448(phbA-2)
BMB: BruAb1_{—}1756(phbA-1) BruAb2_{—}0774(phbA-2)
BOV: BOV_{—}1707(phbA-1)
OAN: Oant_{—}1130 Oant_{—}3107 Oant_{—}3718 Oant_{—}4020
BJA: bll0226(atoB) bll3949 bll7400 bll7819 blr3724(phbA)
BRA: BRADO0562(phbA) BRADO0983(pimB) BRADO3110 BRADO3134(atoB)
BBT: BBta_{—}3558 BBta_{—}3575(atoB) BBta_{—}5147(pimB) BBta_{—}7072(pimB) BBta_{—}7614(phbA)
RPA: RPA0513(pcaF) RPA0531 RPA3715(pimB)
RPB: RPB_{—}0509 RPB_{—}0525 RPB_{—}1748
RPC: RPC_{—}0504 RPC_{—}0636 RPC_{—}0641 RPC_{—}0832 RPC_{—}1050 RPC_{—}2005 RPC_{—}2194 RPC_{—}2228
RPD: RPD_{—}0306 RPD_{—}0320 RPD 3105 RPD_{—}3306
RPE: RPE 0168 RPE_{—}0248 RPE 3827
NWI: Nwi_{—}3060
XAU: Xaut_{—}3108 Xaut_{—}4665
CCR: CC 0510 CC_{—}0894 CC_{—}3462
SIL: SPO0142(bktB) SPO0326(phbA) SPO0773 SPO3408
SIT: TM1040_{—}0067 TM1040_{—}2790 TM1040_{—}3026 TM1040_{—}3735
RSP: RSP_{—}0745 RSP_{—}1354 RSP_{—}3184
RSH: Rsph17029_{—}0022 Rsph17029_{—}2401 Rsph17029_{—}3179 Rsph17029_{—}3921
RSQ: Rsph17025_{—}0012 Rsph17025_{—}2466 Rsph17025_{—}2833
JAN: Jann_{—}0262 Jann_{—}0493 Jann_{—}4050
RDE: RD1_{—}0025 RD1_{—}0201(bktB) RD1_{—}3394(phbA)
PDE: Pden_{—}2026 Pden_{—}2663 Pden_{—}2870 Pden_{—}2907 Pden_{—}4811 Pden_{—}5022
DSH: Dshi_{—}0074 Dshi_{—}3066 Dshi_{—}3331
MMR: Mmar10_{—}0697
HNE: HNE_{—}2706 HNE_{—}3065 HNE_{—}3133
NAR: Saro_{—}0809 Saro_{—}1069 Saro_{—}1222 Saro_{—}2306 Saro_{—}2349
SAL: Sala_{—}0781 Sala_{—}1244 Sala_{—}2896 Sala_{—}3158
SWI: Swit_{—}0632 Swit_{—}0752 Swit_{—}2893 Swit_{—}3602 Swit_{—}4887 Swit_{—}5019 Swit_{—}5309
ELI: ELI_{—}01475 ELI_{—}06705 ELI_{—}12035
GBE: GbCGDNIH1_{—}0447
ACR: Acry_{—}1847 Acry_{—}2256
RRU: Rru_A0274 Rru_A1380 Rru_A1469 Rru_A1946 Rru_A3387
MAG: amb0842
MGM: Mmc1_{—}1165
ABA: Acid345_{—}3239
BSU: BG11319(mmgA) BG13063(yhfS)
BHA: BH1997 BH2029 BH3801(mmgA)
BAN: BA3687 BA4240 BA5589
BAR: GBAA3687 GBAA4240 GBAA5589
BAA: BA_{—}0445 BA_{—}4172 BA_{—}4700
BAT: BAS3418 BAS3932 BAS5193
BCE: BC3627 BC4023 BC5344
BCA: BCE_{—}3646 BCE_{—}4076 BCE_{—}5475
BCZ: BCZK3329(mmgA) BCZK3780(th1) BCZK5044(atoB)
BCY: Bcer98_{—}2722 Bcer98_{—}3865
BTK: BT9727_{—}3379(mmgA) BT9727_{—}3765(thl) BT9727_{—}5028(atoB)
BTL: BALH_{—}3262(mmgA) BALH_{—}3642(fadA) BALH_{—}4843(atoB)
BLI: BL03925(mmgA)
BLD: BLi03968(mmgA)
BCL: ABC0345 ABC2989 ABC3617 ABC3891(mmgA)
BAY: RBAM_{—}022450
BPU: BPUM_{—}2374(yhfS) BPUM_{—}2941 BPUM_{—}3373
OIH: OB0676 OB0689 OB2632 OB3013
GKA: GK1658 GK3397
SAU: SA0342 SA0534(vraB)
SAV: SAV0354 SAV0576(vraB)
SAM: MW0330 MW0531 (vraB)
SAR: SAR0351(th1) SAR0581
SAS: SAS0330 SAS0534
SAC: SACOL0426 SACOL0622(atoB)
SAB: SAB0304(th1) SAB0526
SAA: SAUSA300_{—}0355 SAUSA300_{—}0560(vraB)
SAO: SAOUHSC_{—}00336 SAOUHSC_{—}00558
SAJ: SaudH9_{—}0402
SAH: SaurJH1_{—}0412
SEP: SE0346 SE2384
SER: SERP0032 SERP0220
SHA: SH0510(mvaC) SH2417
SSP: SSP0325 SSP2145
LMO: lmo1414
LMF: LMOf2365_{—}1433
LIN: lin1453
LWE: lwe1431
LLA: L11745(thiL) L25946(fadA)
LLC: LACR_{—}1665 LACR_{—}1956
LLM: llmg_{—}0930(thiL)
SPY: SPy_{—}0140 SPy_{—}1637(atoB)
SPZ: M5005_Spy_{—}0119 M5005_Spy_{—}0432 M5005_Spy_{—}1344(atoB)
SPM: spyM18_{—}0136 spyM18_{—}1645(atoB)
SPG: SpyM3_{—}0108 SpyM3_{—}1378(atoB)
SPS: SPs0110 SPs0484
SPH: MGAS10270_Spy0121 MGAS10270_Spy0433 MGAS10270_Spy1461(atoB)
SPI: MGAS10750_Spy0124 MGAS10750_Spy0452 MGAS10750_Spy1453(atoB)
SPJ: MGAS2096_Spy0123 MGAS2096_Spy0451 MGAS2096_Spy1365(atoB)
SPK: MGAS9429_Spy0121 MGAS9429_Spy0431 MGAS9429_Spy1339(atoB)
SPF: SpyM50447(atoB2)
SPA: M6_Spy0166 M6_Spy0466 M6_Spy1390
SPB: M28_Spy0117 M28_Spy0420 M28_Spy1385(atoB)
SAK: SAK_{—}0568
LJO: LJ1609
LAC: LBA0626(thiL)
LSA: LSA1486
LDB: Ldb0879
LBU: LBUL_{—}0804
LBR: LVIS_{—}2218
LCA: LSEI_{—}1787
LGA: LGAS_{—}1374
LRE: Lreu_{—}0052
EFA: EF1364
OOE: OEOE_{—}0529
STH: STH2913 STH725 STH804
CAC: CAC2873 CA_P0078(thiL)
CPE: CPE2195(atoB)
CPF: CPF_{—}2460
CPR: CPR_{—}2170
CTC: CTC00312
CNO: NT01CX_{—}0538 NT01CX_{—}0603
CDF: CD1059(th1A1) CD2676(th1A2)
CBO: CBO3200(th1)
CBE: Cbei_{—}0411 Cbei_{—}3630
CKL: CKL_{—}3696(th1A1) CKL_{—}3697(th1A2) CKL_{—}3698(th1A3)
AMT: Amet_{—}4630
AOE: Clos_{—}0084 Clos_{—}0258
CHY: CHY_{—}1288 CHY_{—}1355(atoB) CHY_{—}1604 CHY_{—}1738
DSY: DSY0632 DSY0639 DSY1567 DSY1710 DSY2402 DSY3302
DRM: Dred_{—}0400 Dred_{—}1491 Dred_{—}1784 Dred 1892
SWO: Swol_{—}0308 Swol_{—}0675 Swol_{—}0789 Swol_{—}1486 Swol_{—}1934 Swol_{—}2051
TTE: TTE0549(paaJ)
MTA: Moth_{—}1260
MTU: Rv1135A Rv1323(fadA4) Rv3546(fadA5)
MTC: MT1365(phbA)
MBO: Mb1167 Mb1358(fadA4) Mb3576(fadA5) Mb3586c(fadA6)
MBB: BCG_{—}1197 BCG_{—}1385(fadA4) BCG_{—}3610(fadA5) BCG_{—}3620c(fadA6)
MLE: ML1158(fadA4)
MPA: MAP2407c(fadA3) MAP2436c(fadA4)
MAV: MAV_{—}1544 MAV_{—}1573 MAV_{—}1863 MAV_{—}5081
MSM: MSMEG_{—}2224 MSMEG_{—}4920
MUL: MUL_{—}0357
MVA: Mvan_{—}1976 Mvan_{—}1988 Mvan_{—}4305 Mvan_{—}4677 Mvan_{—}4891
MGI: Mflv_{—}1347 Mflv_{—}1484 Mflv_{—}2040 Mflv_{—}2340 Mflv_{—}4356 Mflv_{—}4368
MMC: Mmcs_{—}1758 Mmcs_{—}1769 Mmcs_{—}3796 Mmcs_{—}3864
MKM: Mkms_{—}0251 Mkms_{—}1540 Mkms_{—}1805 Mkms_{—}1816 Mkms_{—}2836 Mkms_{—}3159 Mkms_{—}3286 Mkms_{—}3869 Mkms_{—}3938 Mkms_{—}4227 Mkms_{—}4411 Mkms_{—}4580 Mkms_{—}4724 Mkms_{—}4764 Mkms_{—}4776
MJL: Mjls_{—}0231 Mjls_{—}1739 Mjls_{—}1750 Mjls_{—}2819 Mjls_{—}3119 Mjls_{—}3235 Mjls_{—}3800 Mjls_{—}3850 Mjls_{—}4110 Mjls_{—}4383 Mjls_{—}4705 Mjls_{—}4876 Mjls_{—}5018 Mjls_{—}5063 Mjls_{—}5075
CGL: NCgl2309(cg12392)
CGB: cg2625(pcaF)
CEF: CE0731 CE2295
CJK: jk1543(fadA3)
NFA: nfa10750(fadA4)
RHA: RHA1_ro01455 RHA1_ro01623 RHA1_ro01876 RHA1_ro02517(catF) RHA1_ro03022 RHA1_ro03024 RHA1_ro03391 RHA1_ro03892 RHA1_ro04599 RHA1_ro05257 RHA1_ro08871
SCO: SCO5399(SC8F4.03)
SMA: SAV1384(fadA5) SAV2856(fadA1)
ART: Arth_{—}1160 Arth_{—}2986 Arth_{—}3268 Arth_{—}4073
NCA: Noca_{—}1371 Noca_{—}1797 Noca_{—}1828 Noca_{—}2764 Noca_{—}4142
TFU: Tfu_{—}1520 Tfu_{—}2394
FRA: Francci3_{—}3687
FRE: Franean1_{—}1044 Franean1_{—}2711 Franean1_{—}2726 Franean1_{—}3929 Franean1_{—}4037 Franean1_{—}4577
FAL: FRAAL2514 FRAAL2618 FRAAL5910(atoB)
ACE: Ace1_{—}0626 Ace1_{—}0672
SEN: SACE_{—}1192(mmgA) SACE_{—}2736(fadA6) SACE_{—}4011(catF) SACE_{—}6236(fadA4)
STP: Strop_{—}3610
SAQ: Sare_{—}1316 Sare_{—}3991
RXY: Rxyl_{—}1582 Rxyl_{—}1842 Rxyl_{—}2389 Rxyl_{—}2530
FNU: FN0495
BGA: BG0110(fadA)
BAF: BAPKO_{—}0110(fadA)
LIL: LA0457(thiL1) LA0828(thiL2) LA4139(fadA)
LIC: LIC10396(phbA)
LBJ: LBJ_{—}2862(paaJ-4)
LBL: LBL_{—}0209(paaJ-4)
SYN: s1r1993(phaA)
SRU: SRU_{—}1211(atoB) SRU_{—}1547
CHU: CHU_{—}1910(atoB)
GFO: GFO_{—}1507(atoB)
FJO: Fjoh_{—}4612
FPS: FP0770 FP1586 FP1725
RRS: RoseRS_{—}3911 RoseRS_{—}4348
RCA: Rcas_{—}0702 Rcas_{—}3206
HAU: Haur_{—}0522
DRA: DR_{—}1072 DR_{—}1428 DR_{—}1960 DR_{—}2480 DR_A0053
DGE: Dgeo_{—}0755 Dgeo_{—}1305 Dgeo_{—}1441 Dgeo_{—}1883
TTH: TTC0191 TTC0330
TTJ: TTHA0559
TME: Tmel_{—}1134
FNO: Fnod_{—}0314
PMO: Pmob_{—}0515
HMA: rrnAC0896(acaB3) rrnAC2815(aca2) rrnAC3497(yqeF) rrnB0240(aca1) rrnB0242(acaB2) rrnB0309(acaB1)
TAC: Ta0582
TVO: TVN0649
PTO: PTO1505
APE: APE_{—}2108
SSO: SSO2377(acaB-4)
STO: ST0514
SAI: Saci_{—}0963 Saci_{—}1361(acaB1)
MSE: Msed_{—}0656
PAI: PAE1220
PIS: Pis1_{—}0029 Pis1_{—}1301
PCL: Pca1_{—}0781
PAS: Pars_{—}0309 Pars_{—}1071
CMA: Cmaq_{—}1941

### Exemplary HMG-CoA Synthase Nucleic Acids and Polypeptides

HSA: 3157(HMGCS1) 3158(HMGCS2)
PTR: 457169(HMGCS2) 461892(HMGCS1)
MCC: 702553(HMGCS1) 713541(HMGCS2)
MMU: 15360(Hmgcs2) 208715(Hmgcs1)
RNO: 24450(Hmgcs2) 29637(Hmgcs1)
CFA: 479344(HMGCS1) 607923(HMGCS2)
BTA: 407767(HMGCS 1)
SSC: 397673(CH242-38B5.1)
GGA: 396379(HMGCS1)
XLA: 380091(hmgcs1) 447204(MGC80816)
DRE: 394060(hmgcs1)
SPU: 578259(L00578259)
DME: Dmel_CG4311(Hmgs)
CEL: F25B4.6
ATH: AT4G11820(BAP1)
OSA: 4331418 4347614
CME: CMM189C
SCE: YML126C(ERG13)
AGO: AGOS_ADL356C
PIC: PICST_{—}83020
CAL: Ca019_{—}7312(Ca019.7312)
CGR: CAGL0H04081g
SPO: SPAC4F8.14c(hcs)
MGR: MGG_{—}01026
ANI: AN4923.2
AFM: AFUA_{—}3G10660 AFUA_{—}8G07210
AOR: AO090003000611 AO090010000487
CNE: CNCO5080 CNG02670
UMA: UM05362.1
ECU: ECU10_{—}0510
DDI: DDBDRAFT_{—}0217522 DDB_{—}0219924(hgsA)
TET: TTHERM_{—}00691190
TBR: Tb927.8.6110
YPE: YPO1457
YPK: y2712(pksG)
YPM: YP_{—}1349(pksG)
YPA: YPA_{—}0750
YPN: YPN_{—}2521
YPP: YPDSF_{—}1517
YPS: YPTB1475
CBD: COXBU7E912_{—}1931
TCX: Tcr_{—}1719
DNO: DNO 0799
BMA: BMAA1212
BPS: BPSS1002
BPM: BURPS1710b_A2613
BPL: BURPS1106A_A1384
BPD: BURPS668_A1470
BTE: BTH_II1670
MXA: MXAN_{—}3948(tac) MXAN_{—}4267(mvaS)
BSU: BG10926(pksG)
OIH: OB2248
SAU: SA2334(mvaS)
SAV: SAV2546(mvaS)
SAM: MW2467(mvaS)
SAR: SAR2626(mvaS)
SAS: SAS2432
SAC: SACOL2561
SAB: SAB2420(mvaS)
SAA: SAUSA300_{—}2484
SAO: SAOUHSC_{—}02860
SAJ: SaudH9_{—}2569
SAH: SaudH1_{—}2622
SEP: SE2110
SER: SERP2122
SHA: SH0508(mvaS)
SSP: SSP0324
LMO: lmo1415
LMF: LMOf2365_{—}1434(mvaS)
LIN: lin1454
LWE: lwe1432(mvaS)
LLA: L13187(hmcM)
LLC: LACR_{—}1666
LLM: llmg_{—}0929(hmcM)
SPY: SPy_{—}0881(mvaS.2)
SPZ: M5005_Spy_{—}0687(mvaS.1)
SPM: spyM18_{—}0942(mvaS2)
SPG: SpyM3_{—}0600(mvaS.2)
SPS: SPs1253
SPH: MGAS10270_Spy0745(mvaS1)
SPI: MGAS10750_Spy0779(mvaS1)
SPJ: MGAS2096_Spy0759(mvaS1)
SPK: MGAS9429_Spy0743(mvaS1)
SPF: SpyM51121(mvaS)
SPA: M6_Spy0704
SPB: M28_Spy0667(mvaS.1)
SPN: SP_{—}1727
SPR: spr1571(mvaS)
SPD: SPD_{—}1537(mvaS)
SAG: SAG1316
SAN: gbs1386
SAK: SAK_{—}1347
SMU: SMU.943c
STC: str0577(mvaS)
STL: stu0577(mvaS)
STE: STER_{—}0621
SSA: SSA_{—}0338(mvaS)
SSU: SSU05_{—}1641
SSV: SSU98_{—}1652
SGO: SGO_{—}0244
LPL: lp_{—}2067(mvaS)
LJO: LJ1607
LAC: LBA0628(hmcS)
LSA: LSA1484(mvaS)
LSL: LSL_{—}0526
LDB: Ldb0881(mvaS)
LBU: LBUL_{—}0806
LBR: LVIS_{—}1363
LCA: LSEI_{—}1785
LGA: LGAS_{—}1372
LRE: Lreu_{—}0676
PPE: PEPE_{—}0868
EFA: EF1363
OOE: OEOE_{—}0968
LME: LEUM_{—}1184
NFA: nfa22120
SEN: SACE_{—}4570(pksG)
BBU: BB0683
BGA: BG0706
BAF: BAPKO_{—}0727
FJO: Fjoh_{—}0678
HAL: VNG1615G(mvaB)
HMA: rrnAC1740(mvaS)
HWA: HQ2868A(mvaB)
NPH: NP2608A(mvaB_{—}1) NP4836A(mvaB_{—}2)

### Exemplary Hydroxymethylglutaryl-CoA Reductase Nucleic Acids and Polypeptides

HSA: 3156(HMGCR)
PTR: 471516(HMGCR)
MCC: 705479(HMGCR)
MMU: 15357(Hmgcr)
RNO: 25675(Hmgcr)
CFA: 479182(HMGCR)
BTA: 407159(HMGCR)
GGA: 395145(RCJMB04_{—}14m24)
SPU: 373355(LOC373355)
DME: Dmel_CG10367(Hmgcr)
CEL: F08F8.2
OSA: 4347443
SCE: YLR450W(HMG2) YML075C(HMG1)
AGO: AGOS_AER152W
CGR: CAGLOL11506g
SPO: SPCC162.09c(hmg1)
ANI: AN3817.2
AFM: AFUA_{—}1G11230 AFUA_{—}2G03700
AOR: AO090103000311 AO090120000217
CNE: CNF04830
UMA: UM03014.1
ECU: ECU10_{—}1720
DDI: DDB_{—}0191125(hmgA) DDB_{—}0215357(hmgB)
TBR: Tb927.6.4540
TCR: 506831.40 509167.20
LMA: LmjF30.3190
VCH: VCA0723
VCO: VCO395_{—}0662
VVU: VV2_{—}0117
VVY: VVA0625
VPA: VPA0968
VFI: VFA0841
PAT: Patl_{—}0427
CBU: CBU_{—}0030 CBU_{—}0610
CBD: COXBU7E912_{—}0151 COXBU7E912_{—}0622(hmgA)
TCX: Tcr_{—}1717
DNO: DNO_{—}0797
CVI: CV_{—}1806
SUS: Acid_{—}5728 Acid_{—}6132
SAU: SA2333(mvaA)
SAV: SAV2545(mvaA)
SAM: MW2466(mvaA)
SAB: SAB2419c(mvaA)
SEP: SE2109
LWE: lwe0819(mvaA)
LLA: L10433(mvaA)
LLC: LACR_{—}1664
LLM: llmg_{—}0931(mvaA)
SPY: SPy_{—}0880(mvaS.1)
SPM: spyM18_{—}0941(mvaS1)
SPG: SpyM3_{—}0599(mvaS.1)
SPS: SPs1254
SPH: MGAS 10270_Spy0744
SPI: MGAS10750_Spy0778
SPJ: MGAS2096_Spy0758
SPK: MGAS9429_Spy0742
SPA: M6_Spy0703
SPN: SP_{—}1726
SAG: SAG1317
SAN: gbs1387
STC: str0576(mvaA)
STL: stu0576(mvaA)
STE: STER_{—}0620
SSA: SSA_{—}0337(mvaA)
LPL: lp_{—}0447(mvaA)
LJO: LJ1608
LSL: LSL_{—}0224
LBR: LVIS_{—}0450
LGA: LGAS_{—}1373
EFA: EF1364
NFA: nfa22110
BGA: BG0708(mvaA)
SRU: SRU_{—}2422
FPS: FP2341
MMP: MMP0087(hmgA)
MMQ: MmarC5_{—}1589
MAC: MA3073(hmgA)
MBA: Mbar_A1972
MMA: MM_{—}0335
MBU: Mbur_{—}1098
MHU: Mhun_{—}3004
MEM: Memar_{—}2365
MBN: Mboo_{—}0137
MTH: MTH562
MST: Msp_{—}0584(hmgA)
MSI: Msm_{—}0227
MKA: MK0355(HMG1)
AFU: AF1736(mvaA)
HAL: VNG1875G(mvaA)
HMA: rrnAC3412(mvaA)
HWA: HQ3215A(hmgR)
NPH: NP0368A(mvaA_{—}2) NP2422A(mvaA_{—}1)
TAC: Ta0406m
TVO: TVN1168
PTO: PTO1143
PAB: PAB2106(mvaA)
PFU: PF1848
TKO: TK0914
RCI: RCIX1027(hmgA) RCIX376(hmgA)
APE: APE_{—}1869
IHO: Igni_{—}0476
HBU: Hbut_{—}1531
SSO: SSO0531
STO: ST1352
SAI: Saci_{—}1359
PAI: PAE2182
PIS: Pis1_{—}0814
PCL: Pca1_{—}1085
PAS: Pars_{—}0796

### Exemplary Mevalonate Kinase Nucleic Acids and Polypeptides

HSA: 4598(MVK)
MCC: 707645(MVK)
MMU: 17855(Mvk)
RNO: 81727(Mvk)
CFA: 486309(MVK)
BTA: 505792(MVK)
GGA: 768555(MVK)
DRE: 492477(zgc:103473)
SPU: 585785(LOC585785)
DME: Dmel_CG33671
OSA: 4348331
SCE: YMR208W(ERG12)
AGO: AGOS_AER335W
PIC: PICST_{—}40742(ERG12)
CGR: CAGLOF03861g
SPO: SPAC13G6.11c
MGR: MGG_{—}06946
ANI: AN3869.2
AFM: AFUA_{—}4G07780
AOR: AO090023000793
CNE: CNK01740
ECU: ECU09_{—}1780
DDI: DDBDRAFT_{—}0168621
TET: TTHERM_{—}00637680
TBR: Tb927.4.4070
TCR: 436521.9 509237.10
LMA: LmjF31.0560
CBU: CBU_{—}0608 CBU_{—}0609
CBD: COXBU7E912_{—}0620(mvk)
LPN: lpg2039
LPF: lp12017
LPP: lpp2022
BBA: Bd1027(lmbP) Bd1630(mvk)
MXA: MXAN_{—}5019(mvk)
OIH: OB0225
SAU: SA0547(mvaK1)
SAV: SAV0590(mvaK1)
SAM: MW0545(mvaK1)
SAR: SAR0596(mvaK1)
SAS: SAS0549
SAC: SACOL0636(mvk)
SAB: SAB0540(mvaK1)
SAA: SAUSA300_{—}0572(mvk)
SAO: SAOUHSC_{—}00577
SEP: SE0361
SER: SERP0238(mvk)
SHA: SH2402(mvaK1)
SSP: SSP2122
LMO: lmo0010
LMF: LMOf2365_{—}0011
LIN: lin0010
LWE: lwe0011(mvk)
LLA: L7866(yeaG)
LLC: LACR_{—}0454
LLM: llmg_{—}0425(mvk)
SPY: SPy_{—}0876(mvaK1)
SPZ: M5005_Spy_{—}0682(mvaK1)
SPM: spyM18_{—}0937(mvaK1)
SPG: SpyM3_{—}0595(mvaK1)
SPS: SPs1258
SPH: MGAS10270_Spy0740(mvaK1)
SPI: MGAS10750_Spy0774(mvaK1)
SPJ: MGAS2096_Spy0753(mvaK1)
SPK: MGAS9429_Spy0737(mvaK1)
SPF: SpyM51126(mvaK1)
SPA: M6_Spy0699
SPB: M28_Spy0662(mvaK1)
SPN: SP_{—}0381
SPR: spr0338(mvk)
SPD: SPD_{—}0346(mvk)
SAG: SAG1326
SAN: gbs1396
SAK: SAK_{—}1357(mvk)
SMU: SMU.181
STC: str0559(mvaK1)
STL: stu0559(mvaK1)
STE: STER_{—}0598
SSA: SSA_{—}0333(mvaK1)
SSU: SSU05_{—}0289
SSV: SSU98_{—}0285
SGO: SGO_{—}0239(mvk)
LPL: lp_{—}1735(mvaK1)
LJO: LJ1205
LAC: LBA1167(mvaK)
LSA: LSA0908(mvaK1)
LSL: LSL_{—}0685(eRG)
LDB: Ldb0999(mvk)
LBU: LBUL_{—}0906
LBR: LVIS_{—}0858
LCA: LSEI_{—}1491
LGA: LGAS_{—}1033
LRE: Lreu_{—}0915
PPE: PEPE_{—}0927
EFA: EF0904(mvk)
OOE: OEOE_{—}1100
LME: LEUM_{—}1385
NFA: nfa22070
BGA: BG0711
BAF: BAPKO_{—}0732
FPS: FP0313
MMP: MMP1335
MAE: Maeo_{—}0775
MAC: MA0602(mvk)
MBA: Mbar_A1421
MMA: MM_{—}1762
MBU: Mbur_{—}2395
MHU: Mhun_{—}2890
MEM: Memar_{—}1812
MBN: Mboo_{—}2213
MST: Msp_{—}0858(mvk)
MSI: Msm_{—}1439
MKA: MK0993(ERG12)
HAL: VNG1145G(mvk)
HMA: rrnAC0077(mvk)
HWA: HQ2925A(mvk)
NPH: NP2850A(mvk)
PTO: PTO1352
PHO: PH1625
PAB: PAB0372(mvk)
PFU: PF1637(mvk)
TKO: TK1474
RCI: LRC399(mvk)
APE: APE_{—}2439
HBU: Hbut_{—}0877
SSO: SSO0383
STO: ST2185
SAI: Saci_{—}2365(mvk)
MSE: Msed_{—}1602
PAI: PAE3108
PIS: Pisl_{—}0467
PCL: Pcal_{—}1835
   Exemplary Mevalonate Kinase Nucleic Acids and Polypeptides Homologus to Methanosarcina mazei Mevalonate Kinase
NP_{—}633786.1 mevalonate kinase Methanosarcina mazei Go1
YP_{—}304960.1 mevalonate kinase Methanosarcina barkeri str. Fusaro
NP_{—}615566.1 mevalonate kinase Methanosarcina acetivorans C2A
YP_{—}566996.1 mevalonate kinase Methanococcoides burtonii DSM 6242
YP_{—}684687.1 mevalonate kinase uncultured methanogenic archaeon RC-I
YP_{—}183887.1 mevalonate kinase Thermococcus kodakarensis KOD1
NP_{—}126232.1 mevalonate kinase Pyrococcus abyssi GE5
NP_{—}143478.1 mevalonate kinase Pyrococcus horikoshii OT3
NP_{—}579366.1 mevalonate kinase Pyrococcus furiosus DSM 3638
YP_{—}842907.1 mevalonate kinase Methanosaeta thermophila PT
YP_{—}327075.1 mevalonate kinase Natronomonas pharaonis DSM 2160
YP_{—}658630.1 mevalonate kinase Haloquadratum walsbyi DSM 16790
YP_{—}134862.1 mevalonate kinase Haloarcula marismortui ATCC 43049
YP_{—}001405370.1 mevalonate kinase Candidatus Methanoregula boonei 6A8
YP_{—}001030120.1 mevalonate kinase Methanocorpusculum labreanum Z
YP_{—}447890.1 putative mevalonate kinase Methanosphaera stadtmanae DSM 3091
YP_{—}920295.1 mevalonate kinase Thermofilum pendens Hrk 5
ZP_{—}02015315.1 mevalonate kinase Halorubrum lacusprofundi ATCC 49239
NP_{—}280049.1 mevalonate kinase Halobacterium sp. NRC-1
YP_{—}001274012.1 mevalonate kinase Methanobrevibacter smithii ATCC 35061
YP_{—}001435347.1 mevalonate kinase Ignicoccus hospitalis KIN4/I
YP_{—}001540788.1 mevalonate kinase Caldivirga maquilingensis IC-167
Q50559 KIME_METTH mevalonate kinase (MK)
NP_{—}275189.1 mevalonate kinase Methanothermobacter thermautotrophicus str.
NP_{—}071114.1 mevalonate kinase (mvk) Archaeoglobus fulgidus DSM 4304
YP_{—}504301.1 mevalonate kinase Methanospirillum hungatei JF-1
YP_{—}001040239.1 mevalonate kinase Staphylothermus marinus F1
YP_{—}001047720.1 mevalonate kinase Methanoculleus marisnigri JR1
NP_{—}614276.1 mevalonate kinase Methanopyrus kandleri AV19
YP_{—}001737496.1 mevalonate kinase Candidatus Korarchaeum cryptofilum OPF8
YP_{—}256937.1 mevalonate kinase Sulfolobus acidocaldarius DSM 639
NP_{—}341921.1 mevalonate kinase Sulfolobus solfataricus P2
YP_{—}001276466.1 mevalonate kinase Roseiflexus sp. RS-1
YP_{—}001581649.1 mevalonate kinase Nitrosopumilus maritimus SCM1
NP_{—}378182.1 hypothetical protein ST2185 Sulfolobus tokodaii str. 7
YP_{—}001547075.1 mevalonate kinase Herpetosiphon aurantiacus ATCC 23779
YP_{—}001056718.1 mevalonate kinase Pyrobaculum calidifontis JCM 11548
YP_{—}001431846.1 mevalonate kinase Roseiflexus castenholzii DSM 13941
YP_{—}001153805.1 mevalonate kinase Pyrobaculum arsenaticum DSM 13514
AAG02440.1AF290093_{—}1 mevalonate kinase Enterococcus faecalis
NP_{—}814642.1 mevalonate kinase Enterococcus faecalis V583
YP_{—}001634502.1 mevalonate kinase Chloroflexus aurantiacus J-10-fl
XP_{—}790690.1 similar to Mevalonate kinase (MK) Strongylocentrotus purpuratus
NP_{—}560495.1 mevalonate kinase Pyrobaculum aerophilum str. IM2
YP_{—}929988.1 mevalonate kinase Pyrobaculum islandicum DSM 4184
ZP_{—}01465063.1 mevalonate kinase Stigmatella aurantiaca DW4/3-1
ZP_{—}01906658.1 mevalonate kinase Plesiocystis pacifica SIR-1
NP_{—}248080.1 mevalonate kinase Methanocaldococcus jannaschii DSM 2661
1KKHA chain A of the Methanococcus jannaschii mevalonate kinase

Exemplary Mevalonate Kinase Nucleic Acids and Polypeptides Homologus to Lactobacillus sakei Mevalonate Kinase
YP_{—}395519.1 mevalonate kinase Lactobacillus sakei subsp. sakei 23K
YP_{—}535578.1 mevalonate kinase Lactobacillus salivarius UCC118
YP_{—}804427.1 mevalonate kinase Pediococcus pentosaceus ATCC 25745
YP_{—}001271514.1 mevalonate kinase Lactobacillus reuteri F275
ZP_{—}03073995.1 mevalonate kinase Lactobacillus reuteri 100-23
YP_{—}795031.1 mevalonate kinase Lactobacillus brevis ATCC 367
ZP_{—}02185318.1 mevalonate kinase Carnobacterium sp. AT7
YP_{—}001844008.1 mevalonate kinase Lactobacillus fermentum IFO 3956
NP_{—}266560.1 mevalonate kinase Lactococcus lactis subsp. lactis 111403
YP_{—}818851.1 mevalonate kinase Leuconostoc mesenteroides subsp. mesenteroides ATCC 8293
NP_{—}785308.1 mevalonate kinase Lactobacillus plantarum WCFS1
ZP_{—}00604007.1 Mevalonate kinase Enterococcus faecium DO
YP_{—}808480.1 mevalonate kinase Lactococcus lactis subsp. cremoris SK11
YP_{—}001031775.1 mevalonate kinase Lactococcus lactis subsp. cremoris MG1363
NP 814642.1 mevalonate kinase Enterococcus faecalis V583
AAG02440.1 AF290093_{—}1 mevalonate kinase Enterococcus faecalis
   Exemplary Mevalonate Kinase Nucleic Acids and Polypeptides Homologus to Streptomyces sp. CL190 Mevalonate Kinase
BAB07790.1 mevalonate kinase Streptomyces sp. CL190
BAD86800.1 mevalonate kinase Streptomyces sp. KO-3988
BAB07817.1 mevalonate kinase Kitasatospora griseola
ABS50475.1 NapT6 Streptomyces sp. CNQ525
ABS50448.1 NapT6 Streptomyces aculeolatus
BAE78977.1 mevalonate kinase Streptomyces sp. KO-3988
CAL34097.1 putative mevalonate kinase Streptomyces cinnamonensis
BAD07375.1 mevalonate kinase Actinoplanes sp. A40644
YP_{—}118418.1 putative mevalonate kinase Nocardia farcinica IFM 10152
YP_{—}818851.1 mevalonate kinase Leuconostoc mesenteroides subsp. mesenteroides ATCC 8293
YP_{—}001620791.1 mevalonate kinase Acholeplasma laidlawii PG-8A
NP_{—}720650.1 putative mevalonate kinase Streptococcus mutans UA159
YP_{—}001031775.1 mevalonate kinase Lactococcus lactis subsp. cremoris MG1363
ZP_{—}02689018.1 mevalonate kinase Listeria monocytogenes FSL J2-071
NP_{—}266560.1 mevalonate kinase Lactococcus lactis subsp. lactis Il1403
YP_{—}395519.1 mevalonate kinase Lactobacillus sakei subsp. sakei 23K
YP_{—}808480.1 mevalonate kinase Lactococcus lactis subsp. cremoris SK11
ZP_{—}01926008.1 mevalonate kinase Listeria monocytogenes FSL N1-017
ZP_{—}01942559.1 mevalonate kinase Listeria monocytogenes HPB2262
YP_{—}012624.1 mevalonate kinase Listeria monocytogenes str. 4b F2365
YP_{—}001727922.1 mevalonate kinase Leuconostoc citreum KM20
NP_{—}469357.1 hypothetical protein lin0010 Listeria innocua Clip11262
ZP_{—}00875673.1 Mevalonate kinase Streptococcus suis 89/1591
ZP_{—}00604007.1 Mevalonate kinase Enterococcus faecium DO
ZP_{—}00230799.1 mevalonate kinase Listeria monocytogenes str. 4b H7858
YP_{—}139080.1 mevalonate kinase Streptococcus thermophilus LMG 18311
YP_{—}140970.1 mevalonate kinase Streptococcus thermophilus CNRZ1066
ZP_{—}01544345.1 mevalonate kinase Oenococcus oeni ATCC BAA-1163
YP_{—}001197657.1 mevalonate kinase Streptococcus suis 05ZYH33
YP_{—}810664.1 mevalonate kinase Oenococcus oeni PSU-1
NP_{—}463543.1 hypothetical protein lmo0010 Listeria monocytogenes EGD-e
YP_{—}848214.1 mevalonate kinase Listeria welshimeri serovar 6b str. SLCC5334
ZP_{—}01695505.1 mevalonate kinase Bacillus coagulans 36D1
YP_{—}804427.1 mevalonate kinase Pediococcus pentosaceus ATCC 25745
YP_{—}820062.1 mevalonate kinase Streptococcus thermophilus LMD-9
NP 814642.1 mevalonate kinase Enterococcus faecalis V583
AAG02440.1 AF290093_{—}1 mevalonate kinase Enterococcus faecalis
YP_{—}598349.1 mevalonate kinase Streptococcus pyogenes MGAS10270
YP_{—}535578.1 mevalonate kinase Lactobacillus salivarius UCC118
YP_{—}001851498.1 mevalonate kinase, Erg12 Mycobacterium marinum M
ZP_{—}01817104.1 mevalonate kinase Streptococcus pneumoniae SP3-BS71
YP_{—}002037061.1 mevalonate kinase Streptococcus pneumoniae G54
NP_{—}357932.1 mevalonate kinase Streptococcus pneumoniae R6
ZP_{—}02710031.1 mevalonate kinase Streptococcus pneumoniae CDC1087-00
NP_{—}344908.1 mevalonate kinase Streptococcus pneumoniae TIGR4
YP_{—}001547075.1 mevalonate kinase Herpetosiphon aurantiacus ATCC 23779
AAG02455.1 AF290099_{—}1 mevalonate kinase Streptococcus pneumoniae
ZP_{—}01819603.1 mevalonate kinase Streptococcus pneumoniae SP6-BS73
YP_{—}001271514.1 mevalonate kinase Lactobacillus reuteri F275
NP_{—}965060.1 mevalonate kinase Lactobacillus johnsonii NCC 533
ZP_{—}02919501.1 hypothetical protein STRINF_{—}00343 Streptococcus infantarius
YP_{—}001034340.1 mevalonate kinase, putative Streptococcus sanguinis SK36
YP_{—}001844008.1 mevalonate kinase Lactobacillus fermentum IFO 3956
ZP_{—}03073995.1 mevalonate kinase Lactobacillus reuteri 100-23
NP_{—}688324.1 mevalonate kinase, putative Streptococcus agalactiae 2603V/R
YP_{—}907150.1 mevalonate kinase, Erg12 Mycobacterium ulcerans Agy99
NP_{—}691146.1 mevalonate kinase Oceanobacillus iheyensis HTE831
YP_{—}795031.1 mevalonate kinase Lactobacillus brevis ATCC 367
YP_{—}002123449.1 mevalonate kinase Mvk Streptococcus equi subsp. zooepidemicus str. MGCS 10565
YP_{—}001449558.1 mevalonate kinase Streptococcus gordonii str. Challis substr. CH1
ZP_{—}02185318.1 mevalonate kinase Carnobacterium sp. AT7
YP_{—}001634502.1 mevalonate kinase Chloroflexus aurantiacus J-10-fl
YP_{—}812921.1 mevalonate kinase Lactobacillus delbrueckii subsp. bulgaricus ATCC BAA-365
YP_{—}814846.1 mevalonate kinase Lactobacillus gasseri ATCC 33323
YP_{—}001987652.1 Mevalonate kinase Lactobacillus casei
YP_{—}618979.1 mevalonate kinase Lactobacillus delbrueckii subsp. bulgaricus ATCC 11842
NP_{—}664399.1 mevalonate kinase Streptococcus pyogenes MGAS315
YP_{—}806709.1 mevalonate kinase Lactobacillus casei ATCC 334
YP_{—}060017.1 mevalonate kinase Streptococcus pyogenes MGAS10394
YP_{—}280130.1 mevalonate kinase Streptococcus pyogenes MGAS6180
NP_{—}269075.1 mevalonate kinase Streptococcus pyogenes M1 GAS
YP_{—}001276466.1 mevalonate kinase Roseiflexus sp. RS-1
NP_{—}607080.1 mevalonate kinase Streptococcus pyogenes MGAS8232
NP_{—}785308.1 mevalonate kinase Lactobacillus plantarum WCFS1
ABH11598.1 GMP synthase, mevalonate kinase Lactobacillus helveticus CNRZ32
YP_{—}001577580.1 mevalonate kinase Lactobacillus helveticus DPC 4571
YP_{—}001431846.1 mevalonate kinase Roseiflexus castenholzii DSM 13941
YP_{—}302212.1 mevalonate kinase Staphylococcus saprophyticus subsp. saprophyticus ATCC 15305
YP_{—}040044.1 mevalonate kinase Staphylococcus aureus subsp. aureus MRSA252
AAG02424.1 AF290087_{—}1 mevalonate kinase Staphylococcus aureus
NP_{—}645362.1 mevalonate kinase Staphylococcus aureus subsp. aureus MW2
ZP_{—}01514039.1 mevalonate kinase Chloroflexus aggregans DSM 9485
YP_{—}194037.1 mevalonate kinase Lactobacillus acidophilus NCFM
YP_{—}254317.1 mevalonate kinase Staphylococcus haemolyticus JCSC1435
YP_{—}187834.1 mevalonate kinase Staphylococcus epidermidis RP62A
AAG02435.1 AF290091_{—}1 mevalonate kinase Staphylococcus epidermidis
YP_{—}183887.1 mevalonate kinase Thermococcus kodakarensis KOD1
NP_{—}143478.1 mevalonate kinase Pyrococcus horikoshii OT3
ZP_{—}00780842.1 mevalonate kinase Streptococcus agalactiae 18RS21
NP_{—}579366.1 mevalonate kinase Pyrococcus furiosus DSM 3638
NP_{—}126232.1 mevalonate kinase Pyrococcus abyssi GE5
NP_{—}371114.1 mevalonate kinase Staphylococcus aureus subsp. aureus Mu50
YP_{—}001040239.1 mevalonate kinase Staphylothermus marinus F1
NP_{—}763916.1 mevalonate kinase Staphylococcus epidermidis ATCC 12228
YP_{—}633174.1 mevalonate kinase Myxococcus xanthus DK 1622
YP_{—}920295.1 mevalonate kinase Thermofilum pendens Hrk 5
NP_{—}148611.1 mevalonate kinase Aeropyrum pernix K1
NP_{—}633786.1 mevalonate kinase Methanosarcina mazei Go1

### Exemplary Phosphomevalonate Kinase Nucleic Acids and Polypeptides

HSA: 10654(PMVK)
PTR: 457350(PMVK)
MCC: 717014(PMVK)
MMU: 68603(Pmvk)
CFA: 612251(PMVK)
BTA: 513533(PMVK)
DME: Dmel_CG10268
ATH: AT1G31910
OSA: 4332275
SCE: YMR220W(ERG8)
AGO: AGOS_AER354W
PIC: PICST_{—}52257(ERG8)
CGR: CAGL0F03993g
SPO: SPAC343.01c
MGR: MGG_{—}05812
ANI: AN2311.2
AFM: AFUA_{—}5G10680
AOR: AO090010000471
CNE: CNM00100
UMA: UM00760.1
DDI: DDBDRAFT_{—}0184512
TBR: Tb09.160.3690
TCR: 507913.20 508277.140
LMA: LmjF15.1460
MXA: MXAN_{—}5017
OIH: OB0227
SAU: SA0549(mvaK2)
SAV: SAV0592(mvaK2)
SAM: MW0547(mvaK2)
SAR: SAR0598(mvaK2)
SAS: SAS0551
SAC: SACOL0638
SAB: SAB0542(mvaK2)
SAA: SAUSA300_{—}0574
SAO: SAOUHSC_{—}00579
SAJ: SaudH9_{—}0615
SEP: SE0363
SER: SERP0240
SHA: SH2400(mvaK2)
SSP: SSP2120
LMO: lmo0012
LMF: LMOf2365_{—}0013
LIN: lin0012
LWE: lwe0013
LLA: L10014(yebA)
LLC: LACR_{—}0456
LLM: llmg_{—}0427
SPY: SPy_{—}0878(mvaK2)
SPZ: M5005_Spy_{—}0684(mvaK2)
SPM: spyM18_{—}0939
SPG: SpyM3_{—}0597(mvaK2)
SPS: SPs1256
SPH: MGAS10270_Spy0742(mvaK2)
SPI: MGAS10750_Spy0776(mvaK2)
SPJ: MGAS2096_Spy0755(mvaK2)
SPK: MGAS9429_Spy0739(mvaK2)
SPF: SpyM51124(mvaK2)
SPA: M6_Spy0701
SPB: M28_Spy0664(mvaK2)
SPN: SP_{—}0383
SPR: spr0340(mvaK2)
SPD: SPD_{—}0348(mvaK2)
SAG: SAG1324
SAN: gbs1394
SAK: SAK_{—}1355
SMU: SMU.938
STC: str0561(mvaK2)
STL: stu0561(mvaK2)
STE: STER_{—}0600
SSA: SSA_{—}0335(mvaK2)
SSU: SSU05_{—}0291
SSV: SSU98_{—}0287
SGO: SGO_{—}0241
LPL: lp_{—}1733(mvaK2)
LJO: LJ1207
LAC: LBA1169
LSA: LSA0906(mvaK2)
LSL: LSL_{—}0683
LDB: Ldb0997(mvaK)
LBU: LBUL_{—}0904
LBR: LVIS_{—}0860
LCA: LSEI_{—}1092
LGA: LGAS_{—}1035
LRE: Lreu_{—}0913
PPE: PEPE_{—}0925
EFA: EF0902
NFA: nfa22090
BGA: BG0710
BAF: BAPKO_{—}0731
NPH: NP2852A
SSO: SSO2988
STO: ST0978
SAI: Saci_{—}1244

### Exemplary Diphosphomevalonate Decarboxylase Nucleic Acids and Polypeptides

HSA: 4597(MVD)
PTR: 468069(MVD)
MCC: 696865(MVD)
MMU: 192156(Mvd)
RNO: 81726(Mvd)
CFA: 489663(MVD)
GGA: 425359(MVD)
DME: Dmel_CG8239
SCE: YNR043W(MVD1)
AGO: AGOS_AGL232C
PIC: PICST_{—}90752
CGR: CAGL0CO3630g
SPO: SPAC24C9.03
MGR: MGG_{—}09750
ANI: AN4414.2
AFM: AFUA_{—}4G07130
AOR: AO090023000862
CNE: CNL04950
UMA: UM05179.1
DDI: DDBDRAFT_{—}0218058
TET: TTHERM_{—}00849200
TBR: Tb10.05.0010 Tb10.61.2745
TCR: 507993.330 511281.40
LMA: LmjF18.0020
CBU: CBU_{—}0607(mvaD)
CBD: COXBU7E912_{—}0619(mvaD)
LPN: lpg2040
LPF: lpl2018
LPP: lpp2023
TCX: Tcr_{—}1734
DNO: DNO_{—}0504(mvaD)
BBA: Bd1629
MXA: MXAN_{—}5018(mvaD)
OIH: OB0226
SAU: SA0548(mvaD)
SAV: SAV0591(mvaD)
SAM: MW0546(mvaD)
SAR: SAR0597(mvaD)
SAS: SAS0550
SAC: SACOL0637(mvaD)
SAB: SAB0541(mvaD)
SAA: SAUSA300_{—}0573(mvaD)
SAO: SAOUHSC_{—}00578
SAJ: SaudH9_{—}0614
SAH: SaurJH1_{—}0629
SEP: SE0362
SER: SERP0239(mvaD)
SHA: SH2401(mvaD)
SSP: SSP2121
LMO: lmo0011
LMF: LMOf2365_{—}0012(mvaD)
LIN: lin0011
LWE: lwe0012(mvaD)
LLA: L9089(yeaH)
LLC: LACR_{—}0455
LLM: llmg_{—}0426(mvaD)
SPY: SPy_{—}0877(mvaD)
SPZ: M5005_Spy_{—}0683(mvaD)
SPM: spyM18_{—}0938(mvd)
SPG: SpyM3_{—}0596(mvaD)
SPS: SPs1257
SPH: MGAS10270_Spy0741(mvaD)
SPI: MGAS10750_Spy0775(mvaD)
SPJ: MGAS2096_Spy0754(mvaD)
SPK: MGAS9429_Spy0738(mvaD)
SPF: SpyM51125(mvaD)
SPA: M6_Spy0700
SPB: M28_Spy0663(mvaD)
SPN: SP_{—}0382
SPR: spr0339(mvd1)
SPD: SPD_{—}0347(mvaD)
SAG: SAG1325(mvaD)
SAN: gbs1395
SAK: SAK_{—}1356(mvaD)
SMU: SMU.937
STC: str0560(mvaD)
STL: stu0560(mvaD)
STE: STER_{—}0599
SSA: SSA_{—}0334(mvaD)
SSU: SSU05_{—}0290
SSV: SSU98_{—}0286
SGO: SGO_{—}0240(mvaD)
LPL: lp_{—}1734(mvaD)
LJO: LJ1206
LAC: LBA1168(mvaD)
LSA: LSA0907(mvaD)
LSL: LSL_{—}0684
LDB: Ldb0998(mvaD)
LBU: LBUL_{—}0905
LBR: LVIS_{—}0859
LCA: LSEI_{—}1492
LGA: LGAS_{—}1034
LRE: Lreu_{—}0914
PPE: PEPE_{—}0926
EFA: EF0903(mvaD)
LME: LEUM_{—}1386
NFA: nfa22080
BBU: BB0686
BGA: BG0709
BAF: BAPKO_{—}0730
GFO: GFO_{—}3632
FPS: FP0310(mvaD)
HAU: Haur_{—}1612
HAL: VNG0593G(dmd)
HMA: rrnAC1489(dmd)
HWA: HQ1525A(mvaD)
NPH: NP1580A(mvaD)
PTO: PT00478 PT01356
SSO: SS02989
STO: ST0977
SAI: Saci_{—}1245(mvd)
MSE: Msed_{—}1576
   Exemplary Isopentenyl Phosphate Kinases (IPK) Nucleic Acids and Polypeptides Methanobacterium Thermoautotrophicum gi|2621082
Methanococcus jannaschii DSM 2661 gi|1590842;
Methanocaldococcus jannaschii gi|1590842
Methanothermobacter thermautotrophicus gi|2621082
Picrophilus torridus DSM9790 (IG-57) gi|48477569
Pyrococcus abyssi gi|14520758
Pyrococcus horikoshii OT3 gi|3258052
Archaeoglobus fulgidus DSM4304 gi|2648231

### Exemplary Isopentenyl-Diphosphate Delta-Isomerase (IDI) Nucleic Acids and Polypeptides

HSA: 3422(IDI1) 91734(1DI2)
PTR: 450262(IDI2) 450263(IDI1)
MCC: 710052(LOC710052) 721730(LOC721730)
MMU: 319554(Idi1)
RNO: 89784(Idi1)
GGA: 420459(IDI1)
XLA: 494671(LOC494671)
XTR: 496783(idi2)
SPU: 586184(L00586184)
CEL: K06H7.9(idi-1)
ATH: AT3G02780(IPP2)
OSA: 4338791 4343523
CME: CMB062C
SCE: YPL117C(IDI1)
AGO: AGOS_ADL268C
PIC: PICST_{—}68990(IDI1)
CGR: CAGL0J06952g
SPO: SPBC106.15(idil)
ANI: AN0579.2
AFM: AFUA_{—}6G11160
AOR: AO090023000500
CNE: CNA02550
UMA: UM04838.1
ECU: ECUO2_{—}0230
DDI: DDB_{—}0191342(ipi)
TET: TTHERM_{—}00237280 TTHERM_{—}00438860
TBR: Tb09.211.0700
TCR: 408799.19 510431.10
LMA: LmjF35.5330
EHI: 46.t00025
ECO: b2889(idi)
ECJ: JW2857(idi)
ECE: Z4227
ECS: ECs3761
ECC: c3467
ECI: UTI89_C3274
ECP: ECP_{—}2882
ECV: APECO1_{—}3638
ECW: EcE24377A_{—}3215(idi)
ECX: EcHS_A3048
STY: STY3195
STT: t2957
SPT: SPA2907(idi)
SEC: SC2979(idi)
STM: STM3039(idi)
SFL: SF2875(idi)
SFX: 53074
SFV: SFV_{—}2937
SSN: SSON_{—}3042 SSON_{—}3489(yhfK)
SBO: SBO_{—}3103
SDY: SDY_{—}3193
ECA: ECA2789
PLU: plu3987
ENT: Ent638_{—}3307
SPE: Spro_{—}2201
VPA: VPA0278
VFI: VF0403
PPR: PBPRA0469(mvaD)
PEN: PSEEN4850
CBU: CBU_{—}0607(mvaD)
CBD: COXBU7E912_{—}0619(mvaD)
LPN: lpg2051
LPF: lp12029
LPP: lpp2034
TCX: Tcr_{—}1718
HHA: Hhal_{—}1623
DNO: DNO_{—}0798
EBA: ebA5678 p2A143
DVU: DVU1679(idi)
DDE: Dde_{—}1991
LIP: LI1134
BBA: Bd1626
AFW: Anae109_{—}4082
MXA: MXAN_{—}5021(fni)
RPR: RP452
RTY: RT0439(idi)
RCO: RC0744
RFE: RF_{—}0785(fni)
RBE: RBE_{—}0731(fni)
RAK: A1C_{—}04190
RBO: A1I_{—}04755
RCM: A1E_{—}02555
RRI: A1G_{—}04195
MLO: mlr6371
RET: RHE_PD00245(ypd00046)
XAU: Xaut_{—}4134
SIL: SPO0131
SIT: TM1040_{—}3442
RSP: RSP_{—}0276
RSH: Rsph17029_{—}1919
RSQ: Rsph17025_{—}1019
JAN: Jann_{—}0168
RDE: RD1_{—}0147(idi)
DSH: Dshi_{—}3527
BSU: BG11440(ypgA)
BAN: BA1520
BAR: GBAA1520
BAA: BA_{—}2041
BAT: BAS1409
BCE: BC1499
BCA: BCE_{—}1626
BCZ: BCZK1380(fni)
BCY: Bcer98_{—}1222
BTK: BT9727_{—}1381(fni)
BTL: BALH_{—}1354
BLI: BL02217(fni)
BLD: BLi02426
BAY: RBAM_{—}021020(fni)
BPU: BPUM_{—}2020(fni)
OIH: OB0537
SAU: SA2136(fni)
SAV: SAV2346(fni)
SAM: MW2267(fni)
SAR: SAR2431(fni)
SAS: SAS2237
SAC: SACOL2341(fni)
SAB: SAB2225c(fni)
SAA: SAUSA300_{—}2292(fni)
SAO: SAOUHSC_{—}02623
SEP: SE1925
SER: SERP1937(fni-2)
SHA: SH0712(fni)
SSP: SSP0556
LMO: lmo1383
LMF: LMOf2365_{—}1402(fni)
LIN: lin1420
LWE: lwe1399(fni)
LLA: L11083(yebB)
LLC: LACR_{—}0457
LLM: llmg_{—}0428 (fni)
SPY: SPy_{—}0879
SPZ: M5005_Spy_{—}0685
SPM: spyM18_{—}0940
SPG: SpyM3_{—}0598
SPS: SPs1255
SPH: MGAS 10270_Spy0743
SPI: MGAS10750_Spy0777
SPJ: MGAS2096_Spy0756
SPK: MGAS9429_Spy0740
SPF: SpyM51123(fni)
SPA: M6_Spy0702
SPB: M28_Spy0665
SPN: SP_{—}0384
SPR: spr0341(fni)
SPD: SPD_{—}0349(fni)
SAG: SAG1323
SAN: gbs1393
SAK: SAK_{—}1354(fni)
SMU: SMU.939
STC: str0562(idi)
STL: stu0562(idi)
STE: STER_{—}0601
SSA: SSA_{—}0336
SGO: SGO_{—}0242
LPL: lp_{—}1732(idil)
LJO: LJ1208
LAC: LBA1171
LSA: LSA0905(idi)
LSL: LSL_{—}0682
LDB: Ldb0996(fni)
LBU: LBUL_{—}0903
LBR: LVIS_{—}0861
LCA: LSEI_{—}1493
LGA: LGAS_{—}1036
LRE: Lreu_{—}0912
EFA: EF0901
OOE: OEOE_{—}1103
STH: STH1674
CBE: Cbei_{—}3081
DRM: Dred_{—}0474
SWO: Swo1_{—}1341
MTA: Moth_{—}1328
MTU: Rv1745c(idi)
MTC: MT1787(idi)
MBO: Mb1774c(idi)
MBB: BCG_{—}1784c(idi)
MPA: MAP3079c
MAV: MAV_{—}3894(fni)
MSM: MSMEG_{—}1057(fni) MSMEG_{—}2337(fni)
MUL: MUL_{—}0380(idi2)
MVA: Mvan_{—}1582 Mvan_{—}2176
MGI: Mflv_{—}1842 Mflv_{—}4187
MMC: Mmcs_{—}1954
MKM: Mkms_{—}2000
MJL: Mjls_{—}1934
CGL: NCgl2223(cg12305)
CGB: cg2531(idi)
CEF: CE2207
CDI: DIP1730(idi)
NFA: nfa19790 nfa22100
RHA: RHA1_ro00239
SCO: SC06750(SC5F2A.33c)
SMA: SAV1663(idi)
LXX: Lxx23810(idi)
CMI: CMM_{—}2889(idiA)
AAU: AAur_{—}0321(idi)
PAC: PPA2115
FRA: Francci3_{—}4188
FRE: Franean1_{—}5570
FAL: FRAAL6504(idi)
KRA: Krad_{—}3991
SEN: SACE_{—}2627(idiB_{—}2) SACE_{—}5210(idi)
STP: Strop_{—}4438
SAQ: Sare_{—}4564 Sare_{—}4928
RXY: Rxy1_{—}0400
BBU: BB0684
BGA: BG0707
SYN: sll1556
SYC: syc2161_c
SYF: Synpcc7942_{—}1933
CYA: CYA_{—}2395(fni)
CYB: CYB_{—}2691(fni)
TEL: tll1403
ANA: a114591
AVA: Ava_{—}2461 Ava_B0346
TER: Tery_{—}1589
SRU: SRU_{—}1900(idi)
CHU: CHU_{—}0674(idi)
GFO: GFO_{—}2363(idi)
FJO: Fjoh_{—}0269
FPS: FP1792(idi)
CTE: CT0257
CCH: Cag_{—}1445
CPH: Cpha266_{—}0385
PVI: Cvib_{—}1545
PLT: Plut_{—}1764
RRS: RoseRS_{—}2437
RCA: Rcas_{—}2215
HAU: Haur_{—}4687
DRA: DR_{—}1087
DGE: Dgeo_{—}1381
TTH: TT_P0067
TTJ: TTHB110
MJA: MJ0862
MMP: MMP0043
MMQ: MmarC5_{—}1637
MMX: MmarC6_{—}0906
MMZ: MmarC7_{—}1040
MAE: Maeo_{—}1184
MVN: Mevan_{—}1058
MAC: MA0604(idi)
MBA: Mbar_A1419
MMA: MM_{—}1764
MBU: Mbur_{—}2397
MTP: Mthe_{—}0474
MHU: Mhun_{—}2888
MLA: Mlab_{—}1665
MEM: Memar_{—}1814
MBN: Mboo_{—}2211
MTH: MTH48
MST: Msp_{—}0856(fni)
MSI: Msm_{—}1441
MKA: MK0776(lldD)
AFU: AF2287
HAL: VNG1818G(idi) VNG6081G(crt_{—}1) VNG6445G(crt_{—}2) VNG7060 VNG7149
HMA: rrnAC3484(idi)
HWA: HQ2772A(idiA) HQ2847A(idiB)
NPH: NP0360A(idiB_{—}1) NP4826A(idiA) NP5124A(idiB_{—}2)
TAC: Ta0102
TVO: TVN0179
PTO: PTO0496
PHO: PH1202
PAB: PAB1662
PFU: PF0856
TKO: TK1470
RCI: LRC397(fni)
APE: APE_{—}1765.1
SMR: Smar_{—}0822
IHO: Igni_{—}0804
HBU: Hbut_{—}0539
SSO: SSO0063
STO: ST2059
SAI: Saci_{—}0091
MSE: Msed_{—}2136
PAI: PAE0801
PIS: Pisl_{—}1093
PCL: Pcal_{—}0017
PAS: Pars 0051
TPE: Tpen_{—}0272

### Exemplary Isoprene Synthase Nucleic Acids and Polypeptides

### Genbank Accession Nos.

AY341431
AY316691
AY279379
AJ457070
AY182241

What is claimed is:

1. Cells in culture comprising a nucleic acid encoding a heterologous isoprene synthase polypeptide and one or more nucleic acids encoding MVA pathway polypeptides, wherein the cells further comprise i) one or more copies of a nucleic acid encoding a mevalonate kinase polypeptide, or ii) a nucleic acid encoding a mevalonate kinase polypeptide under the control of a strong promoter, and wherein the cells express the mevalonate kinase polypeptide at a level that is at least about 2-fold higher than the level of expression in cells that do not comprise one or more copies of a nucleic acid encoding a mevalonate kinase polypeptide or a nucleic acid encoding a mevalonate kinase polypeptide under the control of a strong promoter.

2. The cells of claim 1, wherein the cells produce greater than about 400 nmole/g_{wcm}/hr of isoprene.

3. The cells of claim 1, wherein the mevalonate kinase polypeptide is M. mazei mevalonate kinase.

4. The cells of claim 1, wherein the MVA pathway polypeptide is selected from the group consisting of Lactobacillus mevalonate kinase polypeptide, Lactobacillus sakei mevalonate kinase polypeptide, yeast mevalonate kinase polypeptide, Saccharomyces cerevisiae mevalonate kinase polypeptide, Streptococcus mevalonate kinase polypeptide, Streptococcus pneumoniae mevalonate kinase polypeptide, and Streptomyces mevalonate kinase polypeptide, Streptomyces CL190 mevalonate kinase polypeptide.

5. The cells of claim 1, wherein the MVA pathway polypeptide is a polypeptide from Saccharomyces cerevicia or Enterococcus faecalis.

6. The cells of claim 1, wherein the isoprene synthase polypeptide is a polypeptide from Pueraria or Populus or a hybrid, Populus alba×Populus tremula.

7. The cells of claim 6, wherein the isoprene synthase polypeptide is selected from the group consisting of Pueraria montana or Pueraria lobata, Populus tremuloides, Populus alba, Populus nigra, and Populus trichocarpa.

8. The cells of claim 1, wherein the cells are gram-positive bacterial cells, Streptomyces cells, gram-negative bacterial cells, Escherichia cells, Pantoea cells, fungal cells, filamentous fungal cells, Trichoderma cells, Aspergillus cells, or yeast cells.

9. The cells of claim 8, wherein the cells are selected from the group consisting of Bacillus subtilis, Streptomyces lividans, Streptomyces coelicolor, Streptomyces griseus, Escherichia coli, Pantoea citrea, Trichoderma reesei, Aspergillus oryzae and Aspergillus niger, Saccharomyces cerevisiae and Yarrowia lipolytica.

10. The cells of claim 1, wherein the concentration of MVA is between about 0 to about 120 g/L.

11. A composition for producing isoprene comprising cells of claim 1.

12. A method of producing isoprene, the method comprising (a) culturing cells of claim 1 under suitable culture conditions for the production of isoprene, and (b) producing isoprene.

13. The method of claim 12, wherein the cells in culture produce greater than about 400 nmole/g_{wcm}/hr of isoprene.

14. The method of claim 12, wherein the mevalonate kinase polypeptide is M. mazei mevalonate kinase.

15. The method of claim 12, further comprising recovering the isoprene.

16. A method of manufacturing a tire, wherein the improvement comprises using the cells of claim 1 to produce isoprene for the manufacture of the tire.

17. Use of isoprene prepared by the method of claim 12 in the manufacture of a tire.
